(19) United States
(12) Patent Application Publication
(10) Pub. No.: US 2025/0091989 A1
(43) Pub. Date: Mar. 20, 2025

(54) SMALL MOLECULE PROTEIN SYNTHESIS MODULATORS

(71) Applicant: Interdict Bio, Inc., South San Francisco, CA (US)

(72) Inventors: David Gygi, San Francisco, CA (US); Sogole Sami Bahmanyar, San Francisco, CA (US); Lawrence Hamann, San Francisco, CA (US)

(73) Assignee: Interdict Bio, Inc., South San Francisco, CA (US)

(21) Appl. No.: 18/890,292

(22) Filed: Sep. 19, 2024

Related U.S. Application Data
(60) Provisional application No. 63/583,845, filed on Sep. 19, 2023.

(51) Int. Cl. C07D 207/12; A61K 31/40; A61K 31/4025; A61K 31/403; A61K 31/41; A61K 31/4178; A61K 31/4192; A61K 31/423; A61K 31/4245; A61K 31/427; A61K 31/433; A61K 31/4439; A61K 31/496; A61K 31/497; A61K 31/4985; A61K 31/499; A61K 31/5377; A61K 31/69; C07D 401/14; C07D 403/10; C07D 403/12; C07D 403/14; C07D 405/12; C07D 405/14; C07D 409/12; C07D 413/10; C07D 413/12; C07D 413/14; C07D 417/10; C07D 417/12; C07D 417/14; C07D 487/04; C07D 487/10; C07F 5/02

(52) U.S. Cl. CPC C07D 207/12; A61K 31/40; A61K 31/4025; A61K 31/403; A61K 31/41; A61K 31/4178; A61K 31/4192; A61K 31/423; A61K 31/4245; A61K 31/427; A61K 31/433; A61K 31/4439; A61K 31/496; A61K 31/497; A61K 31/4985; A61K 31/499; A61K 31/5377; A61K 31/69; C07D 401/14; C07D 403/10; C07D 403/12; C07D 403/14; C07D 405/12; C07D 405/14; C07D 409/12; C07D 413/10; C07D 413/12; C07D 413/14; C07D 417/10; C07D 417/12; C07D 417/14; C07D 487/04; C07D 487/10; C07F 5/022

(57) ABSTRACT

The present disclosure provides compounds of the formulae herein (e.g., Formula (I)), and pharmaceutically acceptable salts thereof, which are useful for modulating protein synthesis (e.g., modulating synthesis of BCL-2, MYC, CCND1, MCL-1, ALK, KRAS-G12D). The present disclosure also provides pharmaceutical compositions and kits comprising the compounds, or pharmaceutically acceptable salts thereof, and methods of treating or preventing diseases or disorders (e.g., diseases or disorders associated with BCL-2, MYC, CCND1, MCL-1, ALK, KRAS-G12D) by administering to a subject in need thereof the compounds, or pharmaceutically acceptable salts thereof, or pharmaceutical compositions thereof.

### RELATED APPLICATIONS

This application claims priority under 35 U.S.C. § 119(e) to United States Provisional Patent Application, U.S. Ser. No. 63/583,845, filed Sep. 19, 2023, the entire contents of which are incorporated herein by reference.

### BACKGROUND OF THE INVENTION

Small molecule therapeutics have for decades largely focused on binding to the target protein of interest to inhibit its action, or to induce activation of its function. In the case of inhibition, the net effect of the drug binding to its target is to sequester the target, making it unable to effectively perform its native (or in the case of certain pathologic states, aberrant) function. More recently, small molecule therapeutics have also been developed which alter protein homeostasis. Protein homeostasis refers to the equilibrium between protein synthesis and protein turnover, or degradation. In one instance, the existing pool of a given protein inside the cell may be diminished by accelerating the induction of its degradation. This can be achieved by small molecules that bind to a target and/or an E3 ligase, whereby the net result of binding is the induction of the target's degradation by native cellular machinery, as is the case for both molecular glues and PROteolysis TArgeting ChimeraS (PROTACS). The counterbalancing aspect of protein homeostasis relates to modulating the rate of protein synthesis. In this manner, one means to address specific protein targets is to block their synthesis by inhibition of the translation machinery of the cell. Protein synthesis takes place in the ribosome, where a molecule of mRNA encoding for the protein of interest is translated into the protein through a sequence of steps including initiation, elongation, and termination. Small molecules can bind either to elongation or initiation accessory factors to disable proper assembly and operation of the translation machinery or bind directly inside the ribosome to impede translation. In this way, cellular levels of a given protein can be downregulated to ameliorate diseases arising from an overabundance of pathologic proteins. This has broad applications in all therapeutic areas, including, but not limited to oncology, immunology and inflammation, neurodegeneration, cardiovascular and metabolic diseases, rare genetic diseases, and infectious diseases.

### SUMMARY OF THE INVENTION

The present invention relates to small molecules that modulate protein synthesis by inhibiting the translation machinery. These compounds demonstrate therapeutic utility, including but not limited to, in their ability to kill cancer cells.

In one aspect, the present disclosure provides compounds of Formula (I):

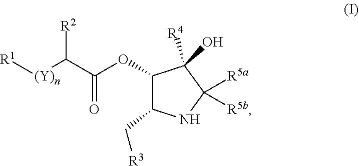

or pharmaceutically acceptable salts thereof, wherein R¹, R², R³, R⁴, R^{5a}, R^{5b}, Y, and n are as defined herein.

In another aspect, the present disclosure provides pharmaceutical compositions comprising a compound disclosed herein. In some embodiments, the pharmaceutical composition comprises an excipient.

In another aspect, the present disclosure provides methods of modulating protein synthesis in a subject in need thereof or in a cell, tissue, or biological sample, comprising administering to the subject in need thereof or contacting the cell, tissue, or biological sample with an effective amount of a provided compound, or a pharmaceutical composition thereof.

In another aspect, the present disclosure provides methods of decreasing protein synthesis in a subject in need thereof or in a cell, tissue, or biological sample, comprising administering to the subject in need thereof or contacting the cell, tissue, or biological sample with an effective amount of a provided compound, or a pharmaceutical composition thereof.

In another aspect, the present disclosure provides methods of treating or preventing a disease or disorder in a subject in need thereof, comprising administering to the subject in need thereof a therapeutically effective amount of a provided compound, or a pharmaceutical composition thereof.

In another aspect, the present disclosure provides kits comprising a provided compound or pharmaceutical composition and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided.

For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”).In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”).

In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₄ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₄ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified

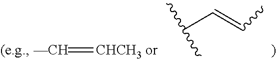

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₄ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl.

Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14π-electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π-electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR)₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl). —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “alkoxy” as used herein, refers to an alkyl group, as defined herein, appended to the parent molecular moiety through an oxygen atom. Representative examples of alkoxy include, but are not limited to, methoxy, ethoxy, n-propoxy, isopropoxy, n-butoxy and tert-butoxy.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{b}b)₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{b})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “boronyl” refers to boranes, boronic acids, boronic esters, borinic acids, and borinic esters, e.g., boronyl groups of the formula —B(R^{aa})₂, —B(OR^{cc})₂, and —BR^{aa}(OR^{cc}), wherein R^{aa} and R^{cc} are as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, Rb, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(Rb)₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(Rb)₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each Rb is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{ff}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R′)₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{a}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The present disclosure is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for modulating protein synthesis (e.g., decreasing protein synthesis). In certain embodiments, a therapeutically effective amount is an amount sufficient for treating a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis). In certain embodiments, a therapeutically effective amount is an amount sufficient for modulating protein synthesis (e.g., decreasing protein synthesis) and a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for modulating protein synthesis (e.g., decreasing protein synthesis). In certain embodiments, a prophylactically effective amount is an amount sufficient for treating a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis). In certain embodiments, a prophylactically effective amount is an amount sufficient for modulating protein synthesis (e.g., decreasing protein synthesis) and a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple-negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), Huntington's disease, and Friedreich's ataxia. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; cerebellar ataxia; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

A “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. These exemplary human protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRK1A, DYRK1B, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN₂, GCN₂₂, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN₂, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK(e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “inhibition,” “inhibiting,” “inhibit,” or “inhibitor” refer to the ability of a compound to reduce, slow, halt or prevent activity of a particular biological process (e.g., protein activity, protein synthesis) in a cell relative to vehicle.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

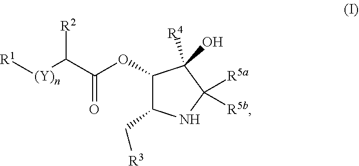

or a pharmaceutically acceptable salt thereof, wherein:
R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂;
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring;
R² is hydrogen or optionally substituted alkyl;
or R¹ and R² are joined together with their intervening atoms to form optionally substituted carbocyclyl or optionally substituted heterocyclyl;
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl;
each instance of Y is independently —C(R^{Y})₂—, —O—, —N(R^{1a})—, or —S—;
each instance of R^{Y} is independently hydrogen or halogen, or two instances of R^{Y} are taken together to form ═O; and
n is 0, 1, 2, 3, 4, or 5.
   R¹, R², Y, R^{1a}, R^{Y} and n

As generally described herein, R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂; and R² is hydrogen or optionally substituted alkyl; or R¹ and R² are joined together with their intervening atoms to form optionally substituted carbocyclyl or optionally substituted heterocyclyl.

In some embodiments, R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted alkenyl, optionally substituted alkynyl, —OR^{1a}, or —N(R^{1a})₂. In some embodiments, R¹ is hydrogen, optionally substituted alkyl, optionally substituted alkenyl, or optionally substituted alkynyl. In some embodiments, R¹ is hydrogen, optionally substituted alkyl, —OR^{1a}, or —N(R^{1a})₂. In some embodiments, R¹ is hydrogen or optionally substituted alkyl. In some embodiments, R¹ is hydrogen, optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted carbocyclyl, optionally substituted heterocyclyl, optionally substituted aryl, or optionally substituted heteroaryl. In some embodiments, R¹ is optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is optionally substituted alkyl, optionally substituted carbocyclyl, optionally substituted heterocyclyl, optionally substituted aryl, or optionally substituted heteroaryl. In some embodiments, R¹ is optionally substituted carbocyclyl, optionally substituted heterocyclyl, optionally substituted aryl, or optionally substituted heteroaryl. In some embodiments, R¹ is optionally substituted carbocyclyl or optionally substituted heterocyclyl. In some embodiments, R¹ is optionally substituted aryl or optionally substituted heteroaryl. In some embodiments, R¹ is optionally substituted heterocyclyl, optionally substituted aryl, or optionally substituted heteroaryl. In some embodiments, R¹ is —OR^{1a} or —N(R^{1a})₂.

In some embodiments, R¹ is hydrogen, optionally substituted alkyl, optionally substituted alkenyl, or optionally substituted alkynyl. In some embodiments, R¹ is hydrogen. In some embodiments, R¹ is optionally substituted alkyl. In some embodiments, R¹ is optionally substituted C₁₋₁₂ alkyl. In some embodiments, R¹ is optionally substituted C₁₋₈ alkyl. In some embodiments, R¹ is optionally substituted C₈ alkyl. In some embodiments, R¹ is optionally substituted C₇ alkyl. In some embodiments, R¹ is optionally substituted C₁₋₆ alkyl. In some embodiments, R¹ is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R¹ is substituted C₁₋₆ alkyl. In some embodiments, R¹ is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R¹ is unsubstituted C₁₋₆ alkyl. In some embodiments, R¹ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R¹ is methyl, ethyl, or isopropyl. In some embodiments, R¹ is optionally substituted methyl. In some embodiments, R¹ is substituted methyl. In some embodiments, R¹ is methyl. In some embodiments, R¹ is optionally substituted ethyl. In some embodiments, R¹ is substituted ethyl. In some embodiments, R¹ is ethyl. In some embodiments, R¹ is optionally substituted isopropyl. In some embodiments, R¹ is substituted isopropyl. In some embodiments, R¹ is isopropyl.

In some embodiments, R¹ is alkyl substituted with ═O, ═S, optionally substituted aryl, optionally substituted heteroaryl, —OR^{1a}, —N(R^{1a})₂, and/or halogen. In some embodiments, R¹ is C₁₋₆ alkyl substituted with ═O, ═S, optionally substituted aryl, optionally substituted heteroaryl, —OR^{1a}, —N(R^{1a})₂, and/or halogen. In some embodiments, R¹ is alkyl substituted with ═O, ═S, optionally substituted aryl, optionally substituted heteroaryl, and/or —N(R^{1a})₂. In some embodiments, R¹ is C₁₋₆ alkyl substituted with ═O, ═S, optionally substituted aryl, optionally substituted heteroaryl, and/or —N(R^{1a})₂.

In some embodiments, R¹ is alkyl substituted with ═O or ═S. In some embodiments, R¹ is alkyl substituted with ═O. In some embodiments, R¹ is C₁₋₆ alkyl substituted with ═O. In some embodiments, R¹ is methyl substituted with ═O. In some embodiments, R¹ is ethyl substituted with ═O. In some embodiments, R¹ is optionally substituted n-propyl substituted with ═O.

In some embodiments, R¹ is alkyl substituted with optionally substituted aryl or optionally substituted heteroaryl. In some embodiments, R¹ is alkyl substituted with optionally substituted aryl. In some embodiments, R¹ is alkyl substituted with optionally substituted phenyl. In some embodiments, R¹ is alkyl substituted with phenyl. In some embodiments, R¹ is C₁₋₆ alkyl substituted with optionally substituted aryl or optionally substituted heteroaryl. In some embodiments, R¹ is C₁₋₆ alkyl substituted with optionally substituted phenyl. In some embodiments, R¹ is C₁₋₆ alkyl substituted with phenyl. In some embodiments, R¹ is methyl substituted with phenyl. In some embodiments, R¹ is ethyl substituted with phenyl. In some embodiments, R¹ is optionally substituted propyl substituted with phenyl. In some embodiments, R¹ is alkyl substituted with optionally substituted heteroaryl.

In some embodiments, R¹ is alkyl substituted with —OR^{1a}. In some embodiments, R¹ is C₁₋₆ alkyl substituted with —OR^{1a}. In some embodiments, R¹ is C₁₋₆ alkyl substituted with —OR^{1a}. In some embodiments, R¹ is C₁₋₆ alkyl substituted with —OH. In some embodiments, R¹ is C₁₋₆ alkyl substituted with —OCH₃. In some embodiments, R¹ is methyl substituted with —OR^{1a}. In some embodiments, R¹ is methyl substituted with —OCH₃. In some embodiments, R¹ is ethyl substituted with —OR^{1a}. In some embodiments, R¹ is isopropyl substituted with —OR^{1a}. In some embodiments, R¹ is isopropyl substituted with —OH.

In some embodiments, R¹ is alkyl substituted with —N(R^{1a})₂. In some embodiments, R¹ is C₁₋₆ alkyl substituted with —N(R^{1a})₂. In some embodiments, R¹ is C₁₋₆ alkyl substituted with —N(R^{1a})₂. In some embodiments, R¹ is C₁₋₆ alkyl substituted with —NH₂. In some embodiments, R¹ is methyl substituted with —N(R^{1a})₂. In some embodiments, R¹ is methyl substituted with —NH₂. In some embodiments, R¹ is ethyl substituted with —N(R^{1a})₂. In some embodiments, R¹ is isopropyl substituted with —N(R^{1a})₂.

In some embodiments, R¹ is alkyl substituted with halogen. In some embodiments, R¹ is alkyl substituted with —F, —Cl, or —Br. In some embodiments, R¹ is alkyl substituted with —F. In some embodiments, R¹ is C₁₋₆ alkyl substituted with halogen. In some embodiments, R¹ is C₁₋₆ alkyl substituted with —F, —Cl, or —Br. In some embodiments, R¹ is C₁₋₆ alkyl substituted with —F. In some embodiments, R¹ is —CH₂F. In some embodiments, R¹ is —CHF₂. In some embodiments, R¹ is —CF₃.

In some embodiments, R¹ is optionally substituted alkenyl. In some embodiments, R¹ is optionally substituted C₂₋₁₂ alkenyl. In some embodiments, R¹ is optionally substituted C₂₋₆ alkenyl. In some embodiments, R¹ is substituted C₂₋₆ alkenyl. In some embodiments, R¹ is unsubstituted C₂₋₆ alkenyl. In some embodiments, R¹ is optionally substituted ethenyl, optionally substituted 1-propenyl, optionally substituted 2-propenyl, optionally substituted 1-butenyl, optionally substituted 2-butenyl, optionally substituted pentenyl, or optionally substituted hexenyl. In some embodiments, R¹ is substituted ethenyl, substituted 1-propenyl, substituted 2-propenyl, substituted 1-butenyl, substituted 2-butenyl, substituted pentenyl, or substituted hexenyl. In some embodiments, R¹ is unsubstituted ethenyl, unsubstituted 1-propenyl, unsubstituted 2-propenyl, unsubstituted 1-butenyl, unsubstituted 2-butenyl, unsubstituted pentenyl, or unsubstituted hexenyl.

In some embodiments, R¹ is optionally substituted alkynyl. In some embodiments, R¹ is optionally substituted C₂₋₁₂ alkynyl. In some embodiments, R¹ is optionally substituted C₂₋₆ alkynyl. In some embodiments, R¹ is substituted C₂₋₆ alkynyl. In some embodiments, R¹ is unsubstituted C₂₋₆ alkynyl. In some embodiments, R¹ is optionally substituted ethynyl, optionally substituted 1-propynyl, optionally substituted 2-propynyl, optionally substituted 1-butynyl, optionally substituted 2-butynyl, optionally substituted pentynyl, or optionally substituted hexynyl. In some embodiments, R¹ is substituted ethynyl, substituted 1-propynyl, substituted 2-propynyl, substituted 1-butynyl, substituted 2-butynyl, substituted pentynyl, or substituted hexynyl. In some embodiments, R¹ is unsubstituted ethynyl, unsubstituted 1-propynyl, unsubstituted 2-propynyl, unsubstituted 1-butynyl, unsubstituted 2-butynyl, unsubstituted pentynyl, or unsubstituted hexynyl. In some embodiments, R¹ is optionally substituted ethynyl. In some embodiments, R¹ is substituted ethynyl. In some embodiments, R¹ is ethynyl.

In some embodiments, R¹ is hydrogen, methyl, ethyl, isopropyl,

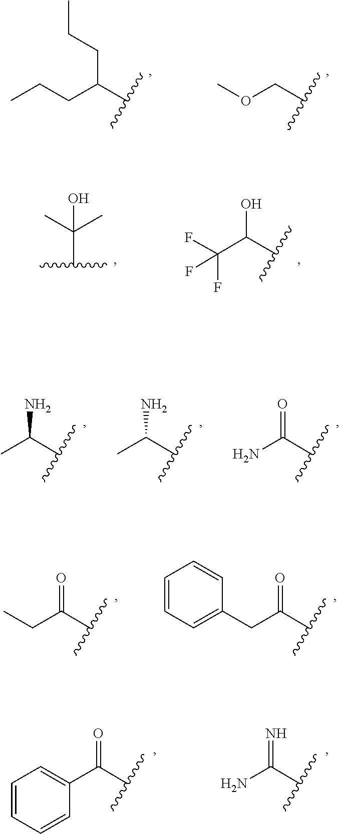

—CHF₂, —CF₃, —CH₂OCF₃, —CH₂SCF₃,

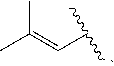 [SMILES: *C=C(C)C]

ethynyl, or

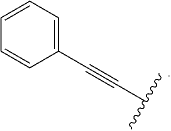 [SMILES: *C#Cc1ccccc1]

embodiments, R¹ is hydrogen, methyl, ethyl, isopropyl,

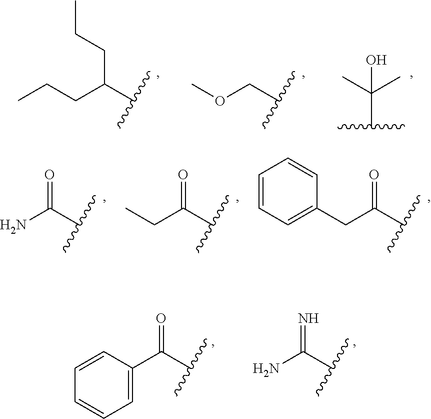

—CF₃, or ethynyl.

In some embodiments, R¹ is hydrogen, methyl, ethyl, isopropyl,

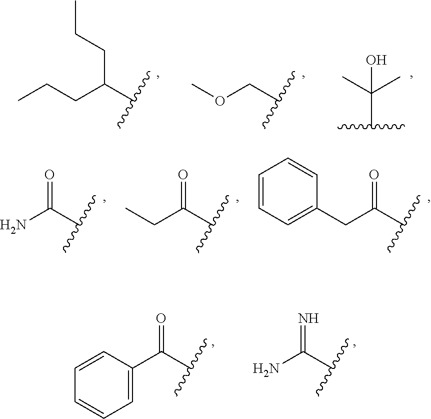

—CF₃, or ethynyl. In some embodiments, R¹ is methyl, ethyl, isopropyl,

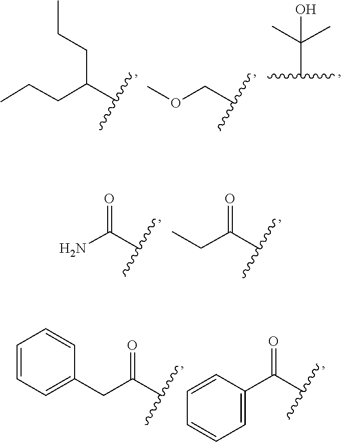

—CF₃, or ethynyl. In some embodiments, R¹ is hydrogen, methyl, ethyl, isopropyl,

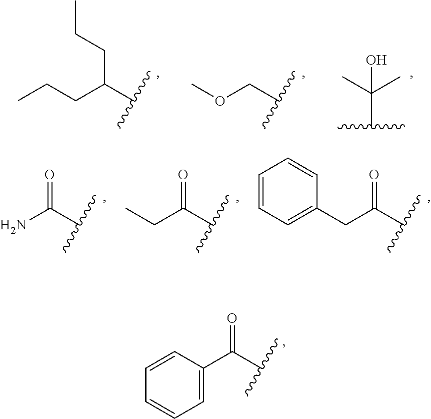

or —CF₃. In some embodiments, R¹ is methyl, ethyl, isopropyl,

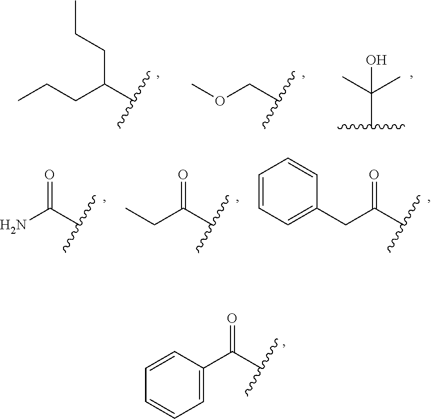

or —CF₃. In some embodiments, R¹ is hydrogen, methyl, ethyl, isopropyl,

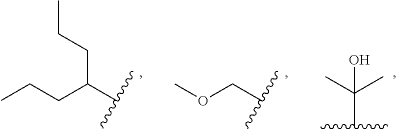

or —CF₃. In some embodiments, R¹ is methyl, ethyl, isopropyl, or

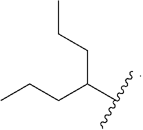 [SMILES: *C(CCC)CCC]

In some embodiments, R¹ is

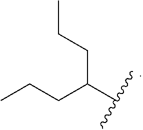 [SMILES: *C(CCC)CCC]

In some embodiments, R¹ is

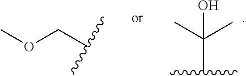

In some embodiments, R¹ is

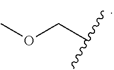 [SMILES: *COC]

In some embodiments, R¹ is

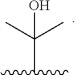 [SMILES: *C(C)(C)O]

In some embodiments, R¹ is

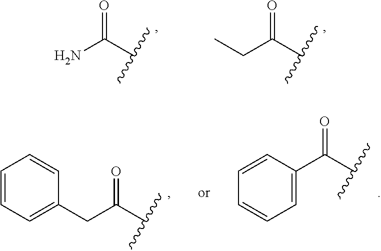

In some embodiments, R¹ is

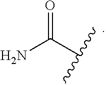 [SMILES: *C(N)=O]

In some embodiments, R¹ is

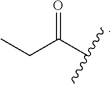 [SMILES: *C(=O)CC]

In some embodiments, R¹ is

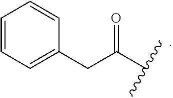 [SMILES: *C(=O)Cc1ccccc1]

In some embodiments, R¹ is

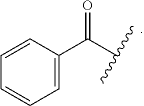 [SMILES: *C(=O)c1ccccc1]

In some embodiments, R¹ is optionally substituted carbocyclyl, optionally substituted heterocyclyl, optionally substituted aryl, or optionally substituted heteroaryl. In some embodiments, R¹ is optionally substituted carbocyclyl or optionally substituted heterocyclyl. In some embodiments, R¹ is optionally substituted carbocyclyl. In some embodiments, R¹ is optionally substituted C₃₋₁₀ carbocyclyl. In some embodiments, R¹ is optionally substituted C₃₋₆ carbocyclyl. In some embodiments, R¹ is optionally substituted cyclopropyl, optionally substituted cyclobutyl, optionally substituted cyclobutenyl, optionally substituted cyclopentyl, or optionally substituted cyclohexyl. In some embodiments, R¹ is optionally substituted cyclopropyl, optionally substituted cyclobutyl, optionally substituted cyclopentyl, or optionally substituted cyclohexyl. In some embodiments, R¹ is substituted cyclopropyl, substituted cyclobutyl, substituted cyclobutenyl, substituted cyclopentyl, or substituted cyclohexyl. In some embodiments, R¹ is substituted cyclopropyl, substituted cyclobutyl, substituted cyclopentyl, or substituted cyclohexyl. In some embodiments, R¹ is substituted cyclopropyl. In some embodiments, R¹ is substituted cyclobutyl. In some embodiments, R¹ is substituted cyclobutenyl. In some embodiments, R¹ is substituted cyclopentyl. In some embodiments, R¹ is substituted cyclohexyl. In some embodiments, R¹ is unsubstituted cyclopropyl, unsubstituted cyclobutyl, unsubstituted cyclobutenyl, unsubstituted cyclopentyl, or unsubstituted cyclohexyl. In some embodiments, R¹ is unsubstituted cyclopropyl. In some embodiments, R¹ is unsubstituted cyclobutyl. In some embodiments, R¹ is unsubstituted cyclobutenyl. In some embodiments, R¹ is unsubstituted cyclopentyl. In some embodiments, R¹ is unsubstituted cyclohexyl.

In some embodiments, R¹ is C₃₋₆ carbocyclyl substituted with halogen, —N(R^{1a})₂, and/or ═O. In some embodiments, R¹ is C₃₋₆ carbocyclyl substituted with halogen. In some embodiments, R¹ is C₃₋₆ carbocyclyl substituted with —F, —Cl, or —Br. In some embodiments, R¹ is C₃₋₆ carbocyclyl substituted with —F. In some embodiments, R¹ is optionally substituted cyclopropyl substituted with halogen. In some embodiments, R¹ is optionally substituted cyclopropyl substituted with —F, —Cl, or —Br. In some embodiments, R¹ is optionally substituted cyclopropyl substituted with —F. In some embodiments, R¹ is cyclobutyl substituted with halogen. In some embodiments, R¹ is cyclobutyl substituted with —F, —Cl, or —Br. In some embodiments, R¹ is cyclobutyl substituted with —F. In some embodiments, R¹ is optionally substituted cyclopentyl substituted with halogen. In some embodiments, R¹ is optionally substituted cyclopentyl substituted with —F, —Cl, or —Br. In some embodiments, R¹ is optionally substituted cyclopentyl substituted with —F. In some embodiments, R¹ is cyclohexyl substituted with halogen. In some embodiments, R¹ is cyclohexyl substituted with —F, —Cl, or —Br. In some embodiments, R¹ is cyclohexyl substituted with —F. In some embodiments, R¹ is

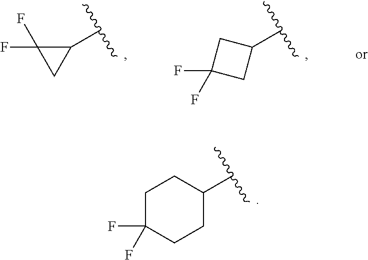 [SMILES: *C1CC1(F)F ; *C1CC(F)(F)C1 ; *C1CCC(F)(F)CC1]

In some embodiments, R¹ is

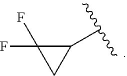 [SMILES: *C1CC1(F)F]

In some embodiments, R¹ is

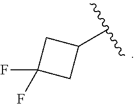 [SMILES: *C1CC(F)(F)C1]

In some embodiments, R¹ is

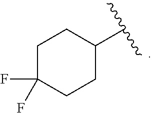 [SMILES: *C1CCC(F)(F)CC1]

In some embodiments, R¹ is C₃₋₆ carbocyclyl substituted with —N(R^{1a})₂. In some embodiments, R¹ is C₃₋₆ carbocyclyl substituted with —NH₂. In some embodiments, R¹ is C₃₋₆ carbocyclyl substituted with —NHR^{1a}. In some embodiments, R¹ is C₃₋₆ carbocyclyl substituted with —NHR^{1a}, and R^{1a} is optionally substituted alkyl. In some embodiments, R¹ is C₃₋₆ carbocyclyl substituted with —NHR^{1a}, and R^{1a} is benzyl. In some embodiments, R¹ is cyclobutenyl substituted with —N(R^{1a})₂. In some embodiments, R¹ is cyclobutenyl substituted with —NH₂. In some embodiments, R¹ is cyclobutenyl substituted with —NHR^{1a}. In some embodiments, R¹ is cyclobutenyl substituted with —NHR^{1a}, and R^{1a} is optionally substituted alkyl. In some embodiments, R¹ is cyclobutenyl substituted with —NHR^{1a}, and R^{1a} is benzyl. In some embodiments, R¹ is C₃₋₆ carbocyclyl substituted with ═O. In some embodiments, R¹ is cyclobutenyl substituted with ═O. In some embodiments, R¹ is

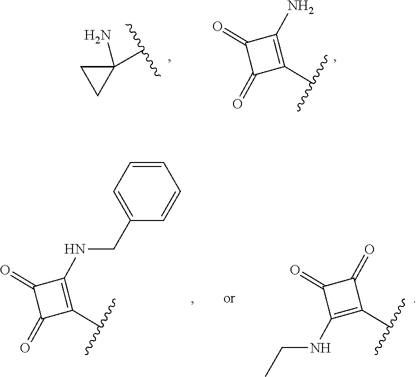

In some embodiments, R¹ is

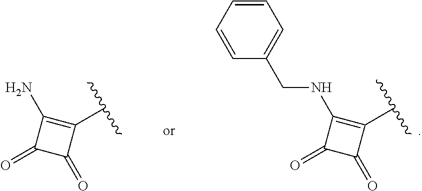 [SMILES: *c1c(N)c(=O)c1=O ; *c1c(NCc2ccccc2)c(=O)c1=O]

In some embodiments, R¹ is

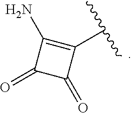 [SMILES: *c1c(N)c(=O)c1=O]

In some embodiments, R¹ is

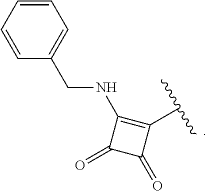 [SMILES: *c1c(NCc2ccccc2)c(=O)c1=O]

In some embodiments, R¹ is optionally substituted cyclopropyl, optionally substituted cyclobutyl, optionally substituted cyclohexyl, or optionally substituted cyclobutenyl. In some embodiments, R¹ is

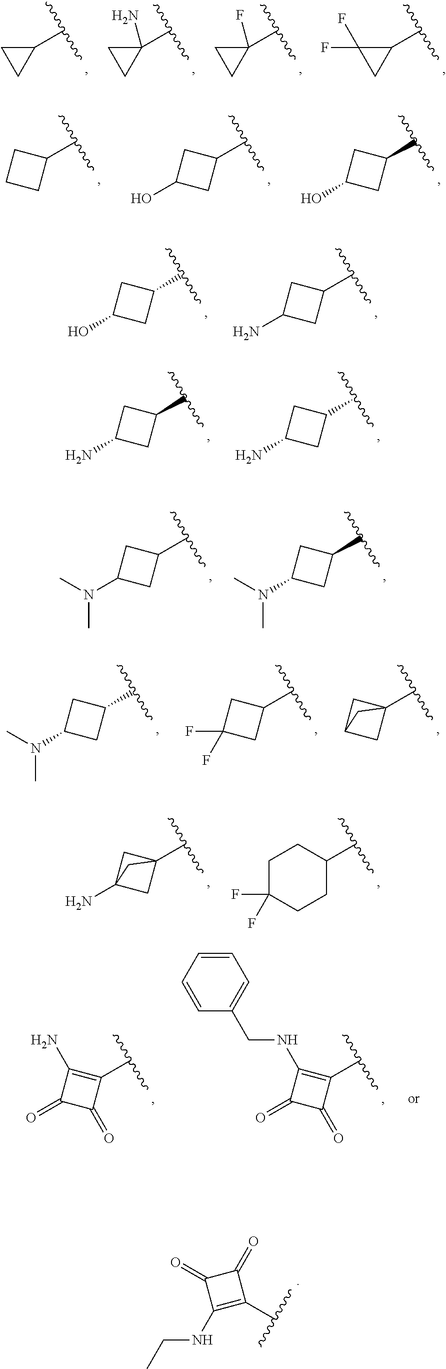

In some embodiments, R¹ is

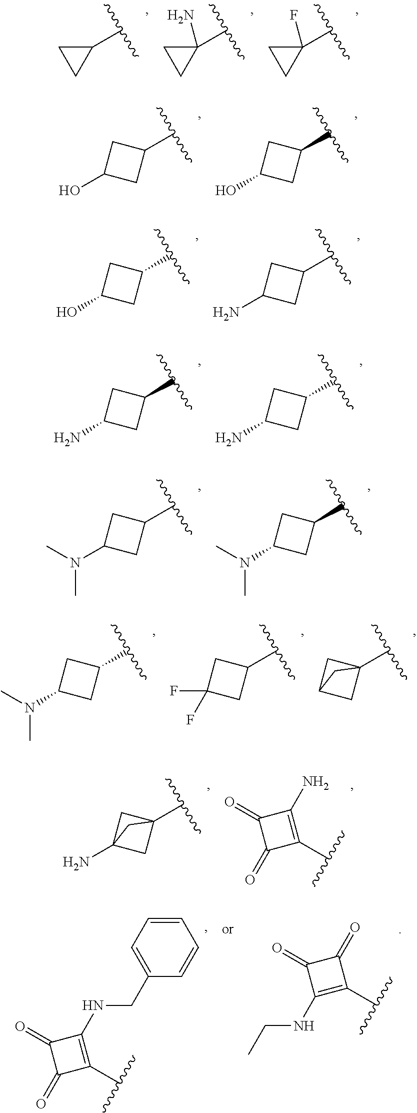

In some embodiments, R¹ is

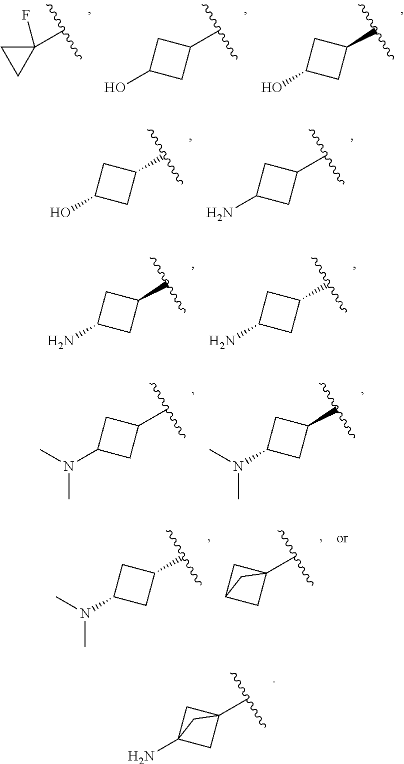

In some embodiments, R¹ is

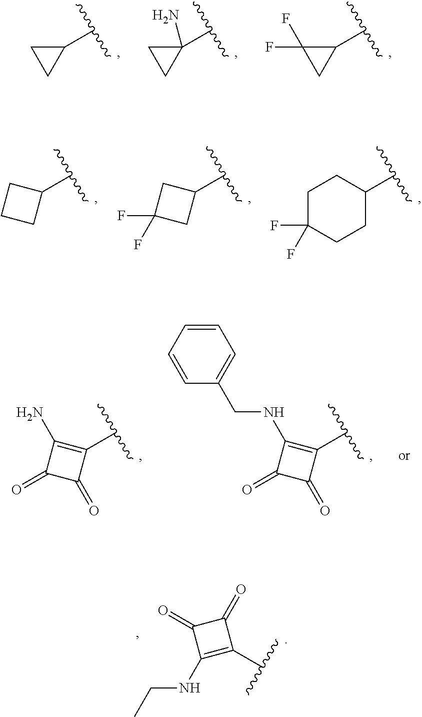

In some embodiments, R¹ is

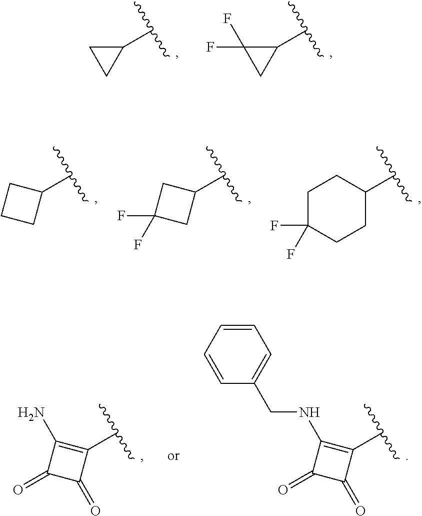

In some embodiments, R¹ is

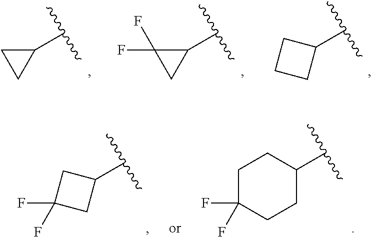

In some embodiments, R¹ is

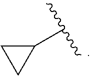 [SMILES: *C1CC1]

In some embodiments, R¹ is

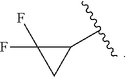 [SMILES: *C1CC1(F)F]

In some embodiments, R¹ is

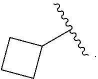 [SMILES: *C1CCC1]

In some embodiments, R¹ is

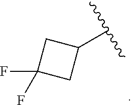 [SMILES: *C1CC(F)(F)C1]

In some embodiments, R¹ is

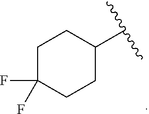 [SMILES: *C1CCC(F)(F)CC1]

In some embodiments, R¹ is

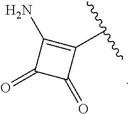 [SMILES: *c1c(N)c(=O)c1=O]

In some embodiments, R¹ is or

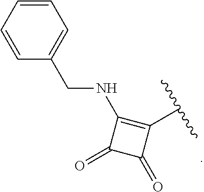 [SMILES: *c1c(NCc2ccccc2)c(=O)c1=O]

In some embodiments, R¹ is optionally substituted heterocyclyl, optionally substituted aryl, or optionally substituted heteroaryl. In some embodiments, R¹ is optionally substituted heterocyclyl. In some embodiments, R¹ is optionally substituted 3-14 membered heterocyclyl. In some embodiments, R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydroimidazopyrazinyl, optionally substituted tetrahydrothiopyranyl, optionally substituted 2,6-diazaspiro[3.3]heptyl, optionally substituted 6-azaspiro[3.4]octyl, 2,6-diazaspiro[3.4]octyl, optionally substituted 2,5,8-triazaspiro[3.5]nonyl, or optionally substituted 1,4,9-triazaspiro[5.5]undecyl. In some embodiments, R¹ is substituted tetrahydrofuranyl, substituted tetrahydropyranyl, substituted azetidinyl, substituted pyrrolidinyl, substituted imidazolidinyl, substituted piperidinyl, substituted piperazinyl, substituted tetrahydroimidazopyrazinyl, substituted tetrahydrothiopyranyl, substituted 2,6-diazaspiro[3.3]heptyl, substituted 6-azaspiro[3.4]octyl, 2,6-diazaspiro[3.4]octyl, substituted 2,5,8-triazaspiro[3.5]nonyl, or substituted 1,4,9-triazaspiro[5.5]undecyl. In some embodiments, R¹ is unsubstituted tetrahydrofuranyl, unsubstituted tetrahydropyranyl, unsubstituted azetidinyl, unsubstituted pyrrolidinyl, unsubstituted imidazolidinyl, unsubstituted piperidinyl, unsubstituted piperazinyl, unsubstituted tetrahydroimidazopyrazinyl, unsubstituted tetrahydrothiopyranyl, unsubstituted 2,6-diazaspiro[3.3]heptyl, unsubstituted 6-azaspiro[3.4]octyl, 2,6-diazaspiro[3.4]octyl, unsubstituted 2,5,8-triazaspiro[3.5]nonyl, or unsubstituted 1,4,9-triazaspiro[5.5]undecyl.

In some embodiments, R¹ is optionally substituted tetrahydropyranyl, optionally substituted azetidinyl, optionally substituted pyrrolidinyl, optionally substituted imidazolidinyl, optionally substituted piperazinyl, optionally substituted tetrahydroimidazopyrazinyl, or optionally substituted 2,5,8-triazaspiro[3.5]nonyl. In some embodiments, R¹ is substituted tetrahydropyranyl, substituted azetidinyl, substituted pyrrolidinyl, substituted imidazolidinyl, substituted piperazinyl, substituted tetrahydroimidazopyrazinyl, or substituted 2,5,8-triazaspiro[3.5]nonyl. In some embodiments, R¹ is unsubstituted tetrahydropyranyl, unsubstituted azetidinyl, unsubstituted pyrrolidinyl, unsubstituted imidazolidinyl, unsubstituted piperazinyl, unsubstituted tetrahydroimidazopyrazinyl, or unsubstituted 2,5,8-triazaspiro[3.5]nonyl. In some embodiments, R¹ is optionally substituted tetrahydropyranyl. In some embodiments, R¹ is optionally substituted azetidinyl. In some embodiments, R¹ is optionally substituted pyrrolidinyl. In some embodiments, R¹ is optionally substituted imidazolidinyl. In some embodiments, R¹ is optionally substituted piperazinyl. In some embodiments, R¹ is optionally substituted tetrahydroimidazopyrazinyl. In some embodiments, R¹ is optionally substituted tetrahydrothiopyranyl. In some embodiments, R¹ is optionally substituted 2,6-diazaspiro[3.3]heptyl. In some embodiments, R¹ is optionally substituted 6-azaspiro[3.4]octyl. In some embodiments, R¹ is optionally substituted 2,6-diazaspiro[3.4]octyl. In some embodiments, R¹ is optionally substituted 2,5,8-triazaspiro[3.5]nonyl. In some embodiments, R¹ is optionally substituted 1,4,9-triazaspiro[5.5]undecyl.

In some embodiments, R¹ is optionally substituted 4-6 membered heterocyclyl. In some embodiments, R¹ is optionally substituted tetrahydropyranyl, optionally substituted azetidinyl, optionally substituted pyrrolidinyl, optionally substituted imidazolidinyl, optionally substituted piperazinyl, or optionally substituted tetrahydrothiopyranyl. In some embodiments, R¹ is optionally substituted azetidinyl, optionally substituted pyrrolidinyl, optionally substituted imidazolidinyl, or optionally substituted piperazinyl.

In some embodiments, R¹ is 3-14 membered heterocyclyl substituted with ═O, optionally substituted alkyl, halogen, —OR^{1a}, —N(R^{1a})₂, and/or —CN. In some embodiments, R¹ is 3-14 membered heterocyclyl substituted with ═O. In some embodiments, R¹ is 3-14 membered heterocyclyl substituted with optionally substituted alkyl. In some embodiments, R¹ is 3-14 membered heterocyclyl substituted with optionally substituted C₁₋₆ alkyl. In some embodiments, R¹ is 3-14 membered heterocyclyl substituted with methyl. In some embodiments, R¹ is 3-14 membered heterocyclyl substituted with ═O and optionally substituted alkyl. In some embodiments, R¹ is 3-14 membered heterocyclyl substituted with ═O and methyl. In some embodiments, R¹ is 3-14 membered heterocyclyl substituted with halogen. In some embodiments, R¹ is 3-14 membered heterocyclyl substituted with —F. In some embodiments, R¹ is 3-14 membered heterocyclyl substituted with —OR^{1a}.

In some embodiments, R¹ is 3-14 membered heterocyclyl substituted with —OH. In some embodiments, R¹ is 3-14 membered heterocyclyl substituted with —N(R^{1a})₂. In some embodiments, R¹ is 3-14 membered heterocyclyl substituted with —CN.

In some embodiments, R¹ is

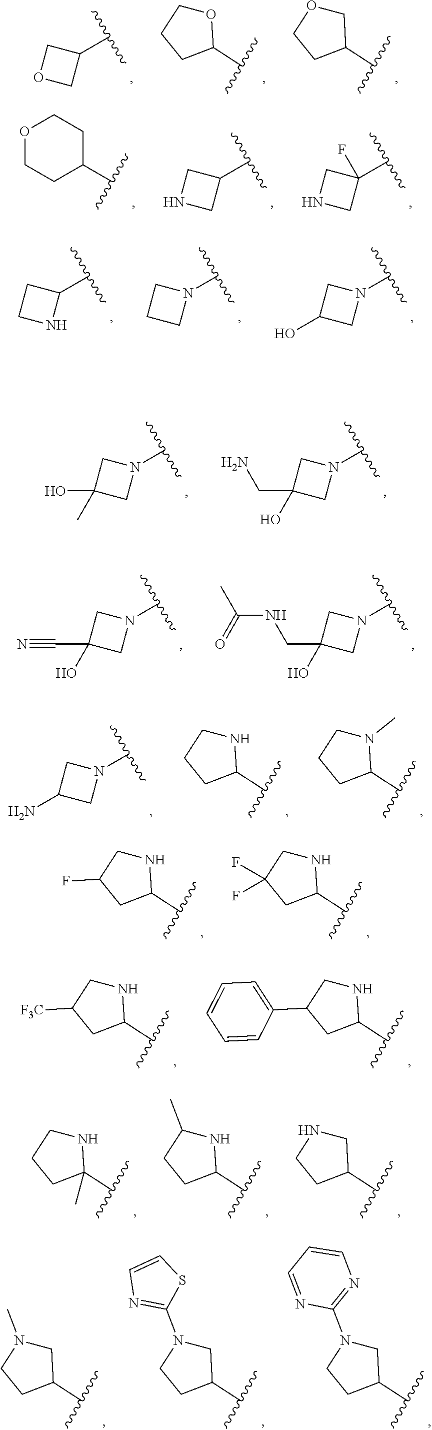

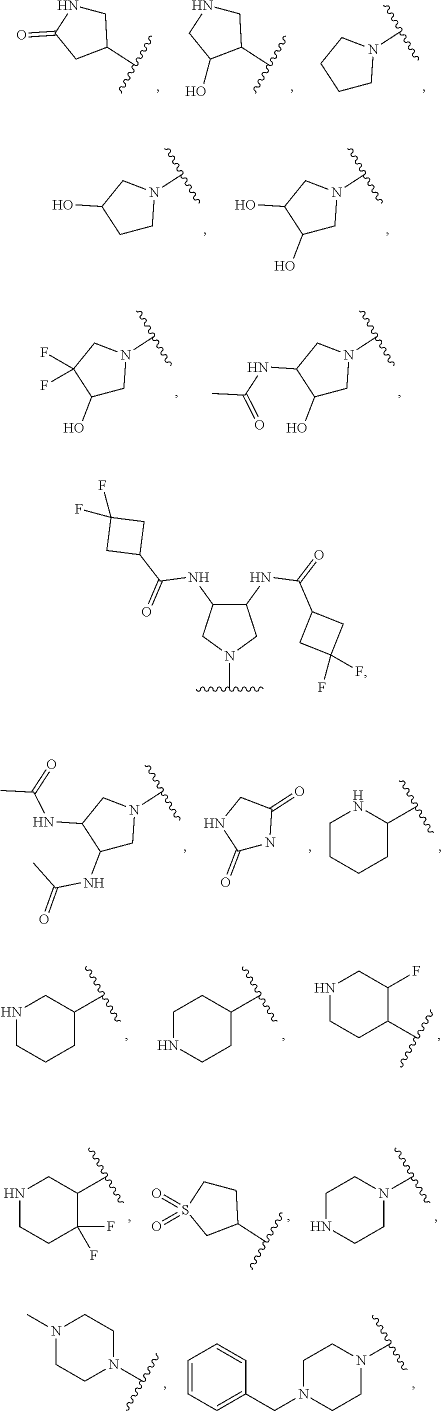

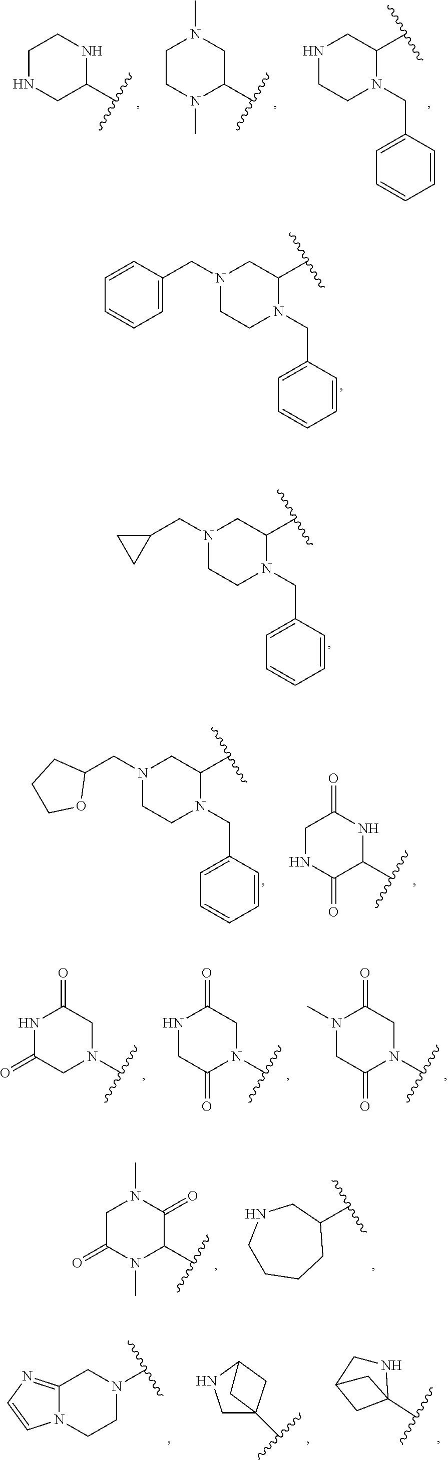

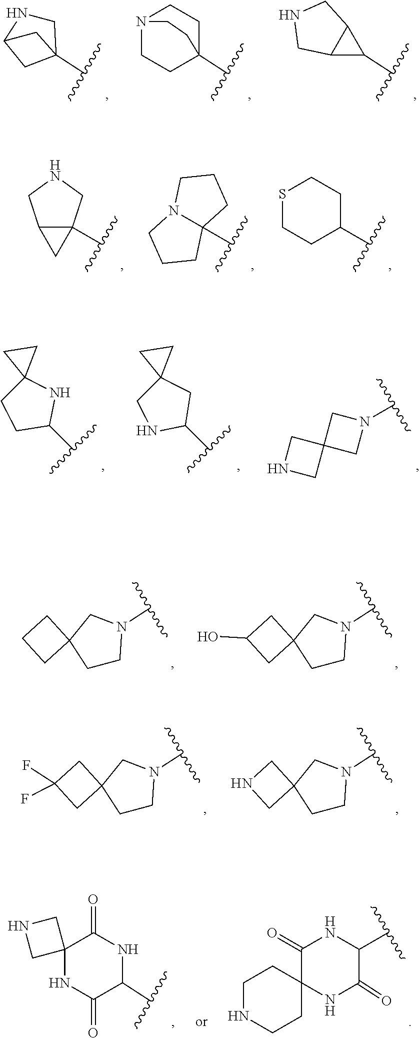

In some embodiments, R¹ is

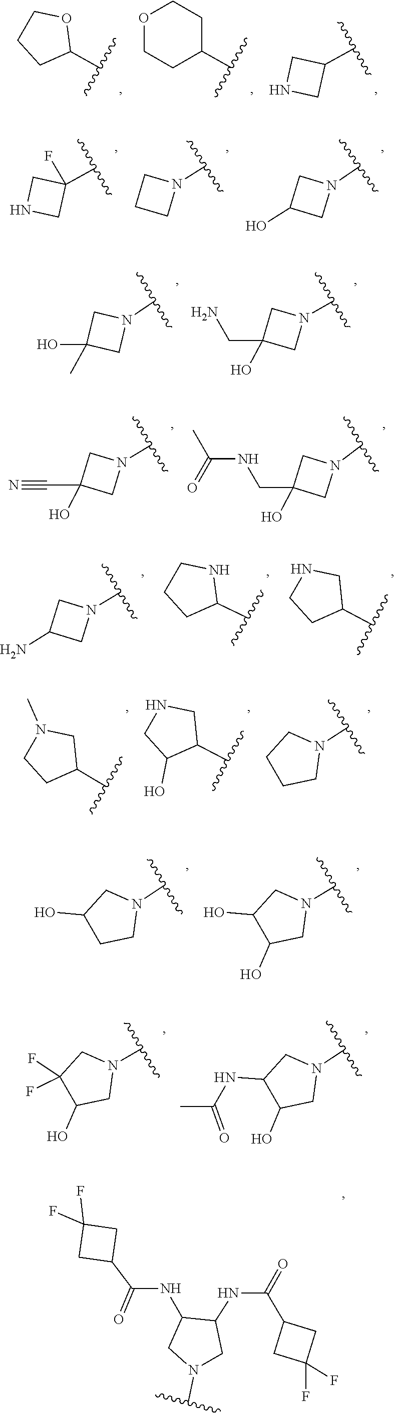

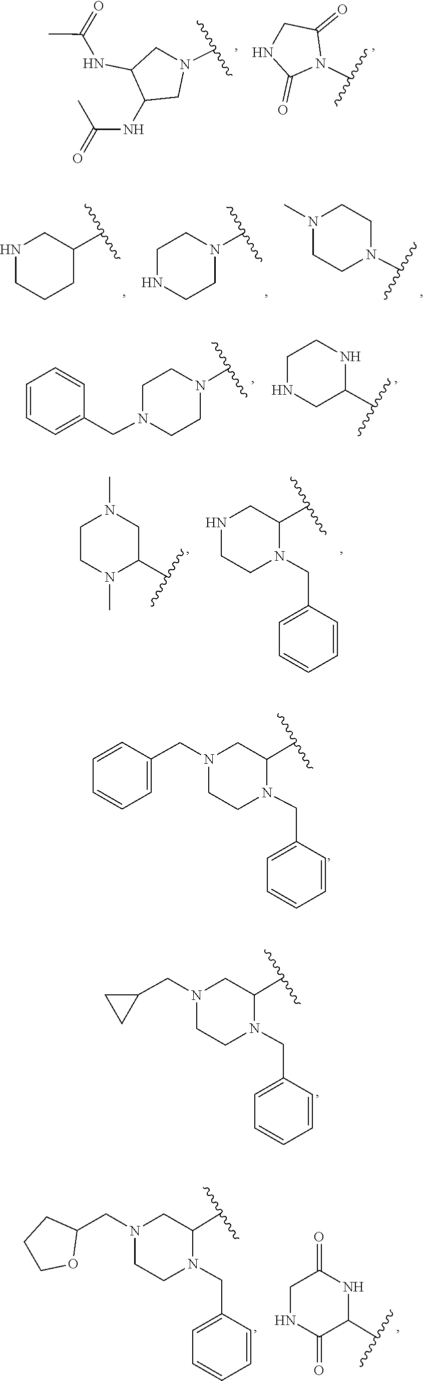

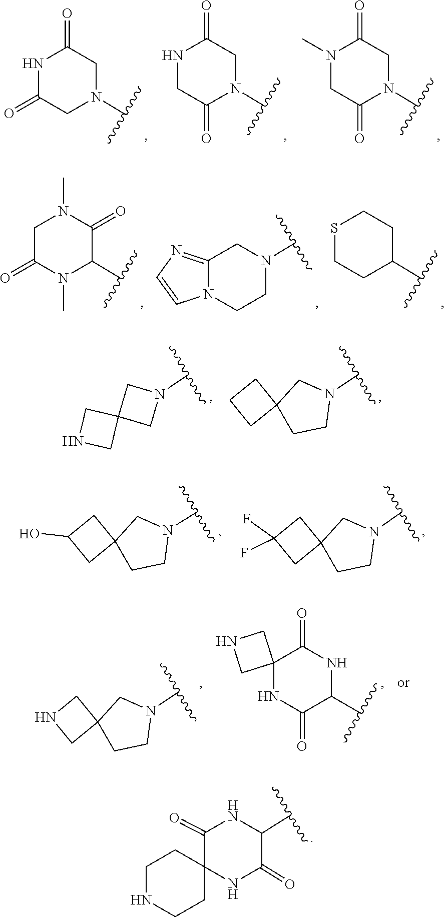

In some embodiments, R¹ is

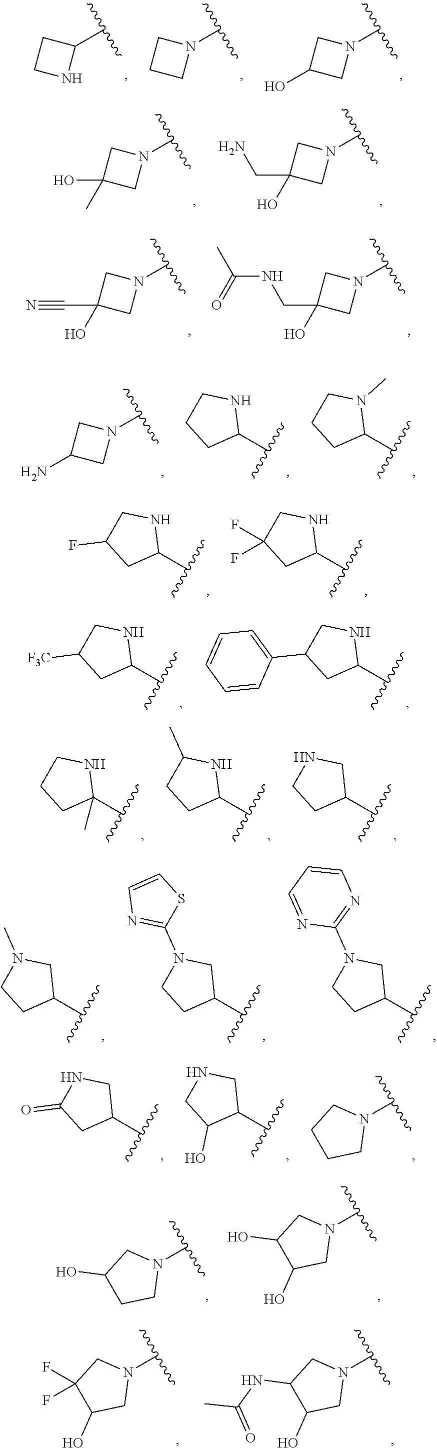

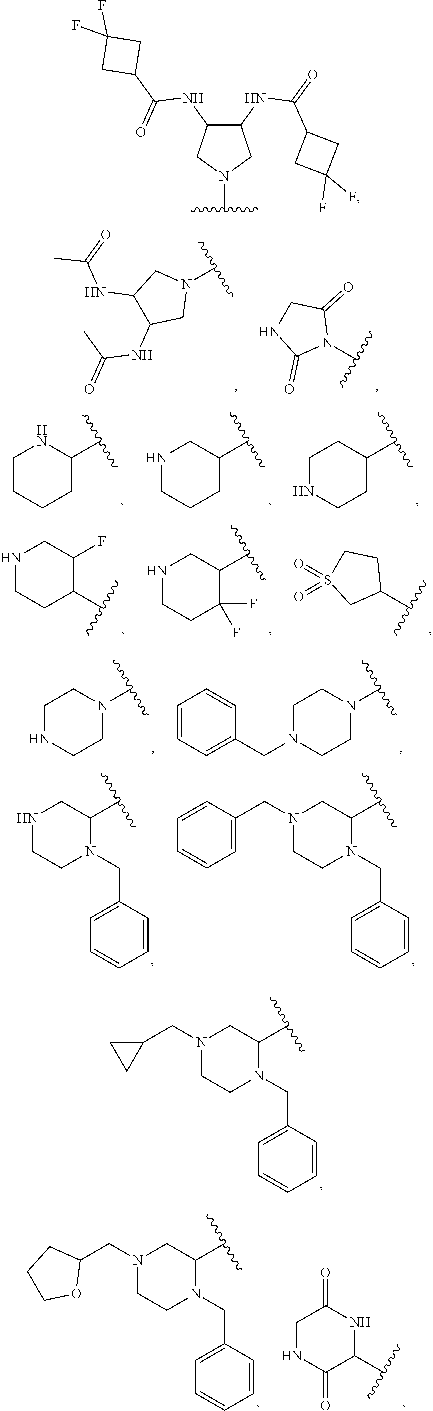

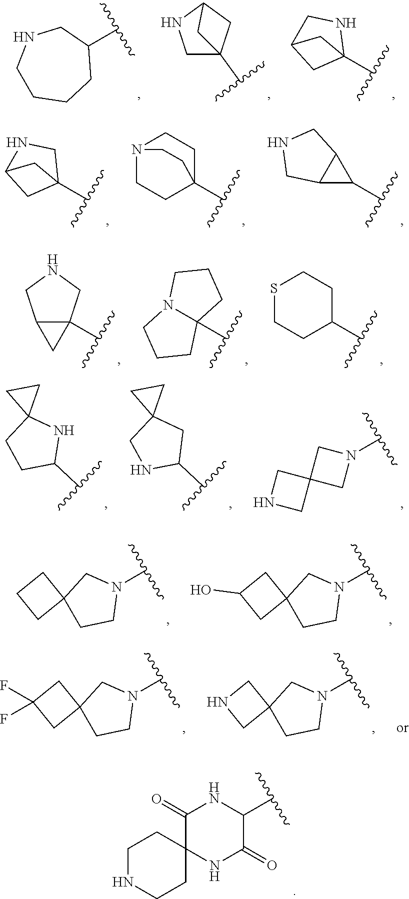

In some embodiments, R¹ is

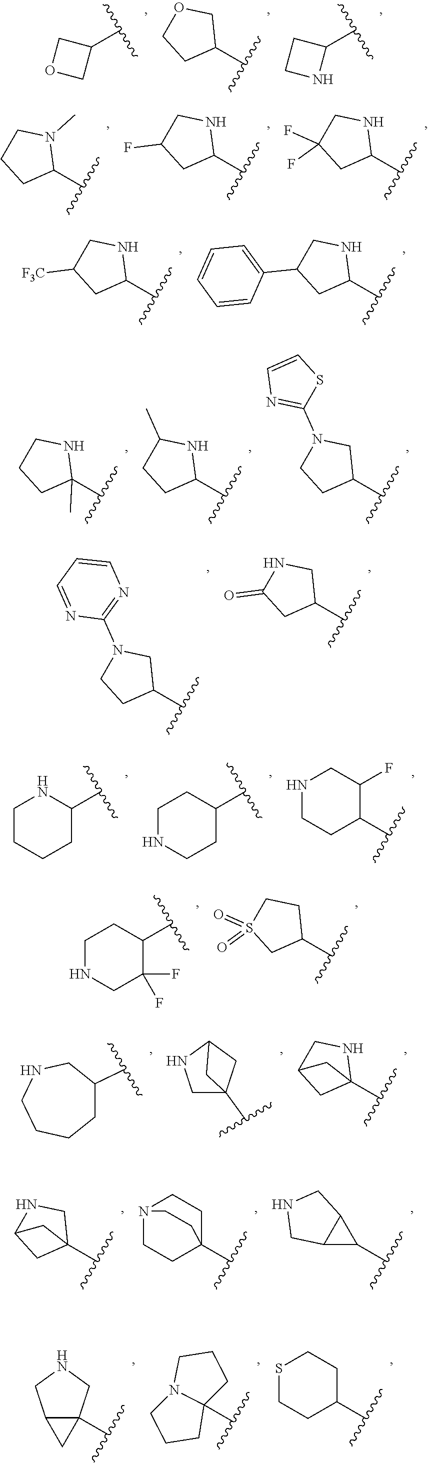

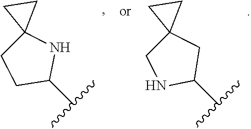 [SMILES: *C1CCC2(CC2)N1 ; *C1CC2(CC2)CN1]

In some embodiments, R¹ is

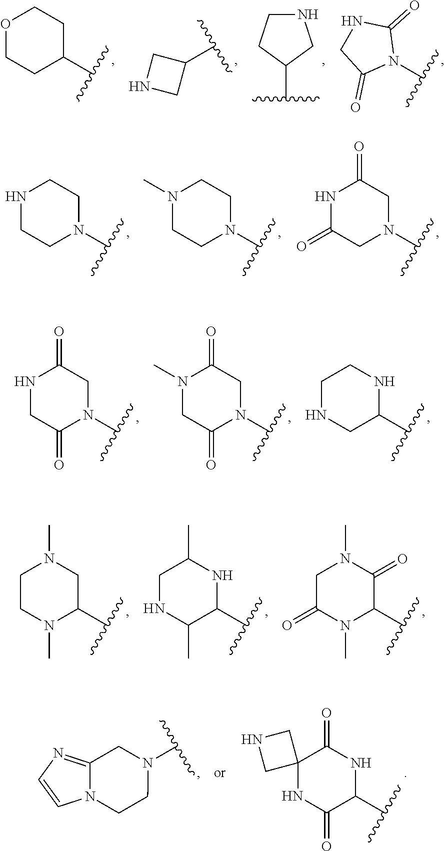

In some embodiments, R¹ is

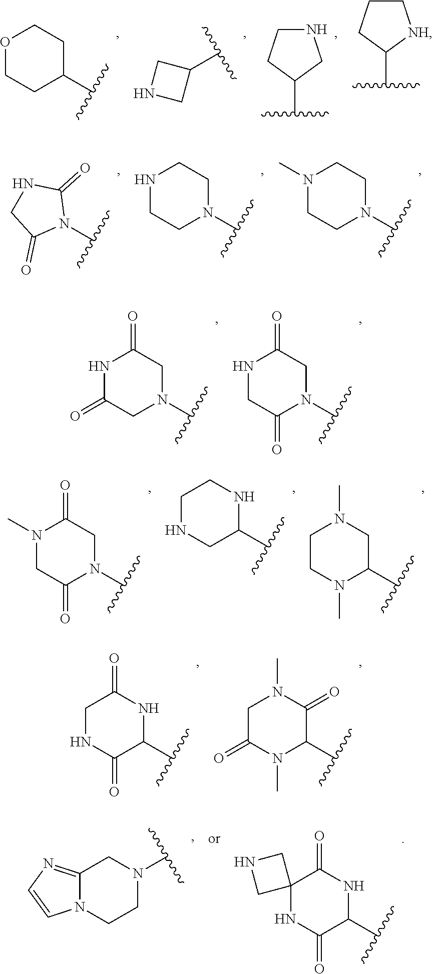

or In some embodiments, R¹ is

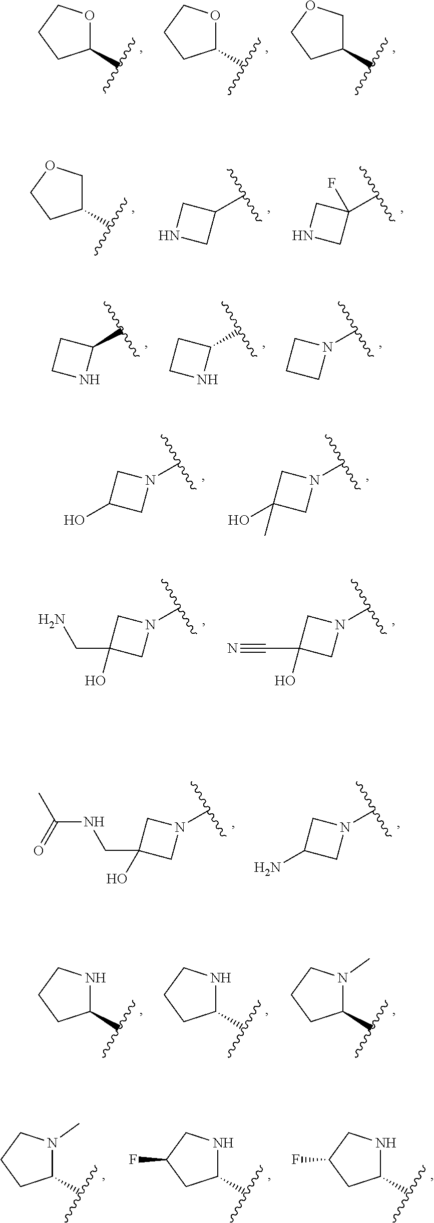

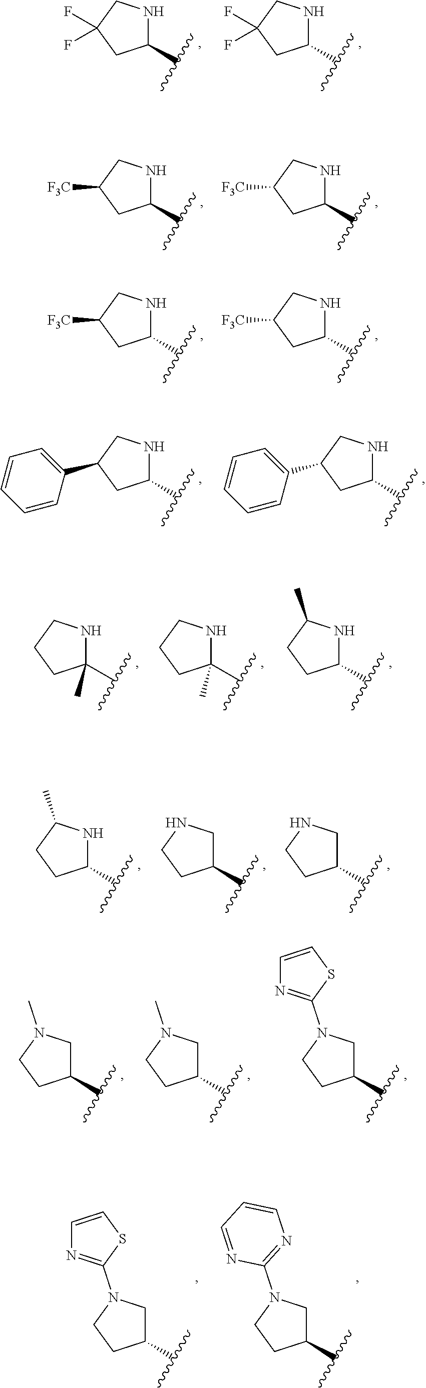

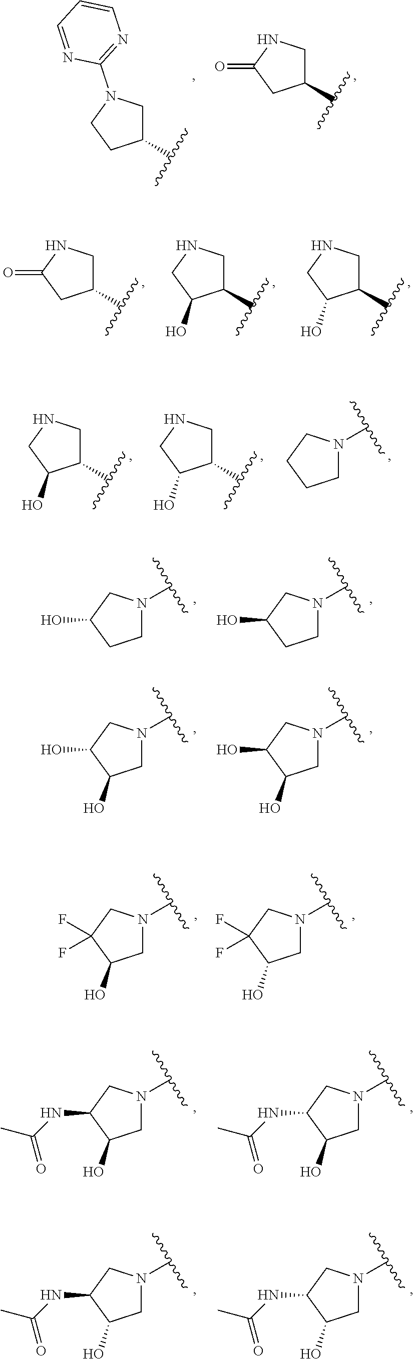

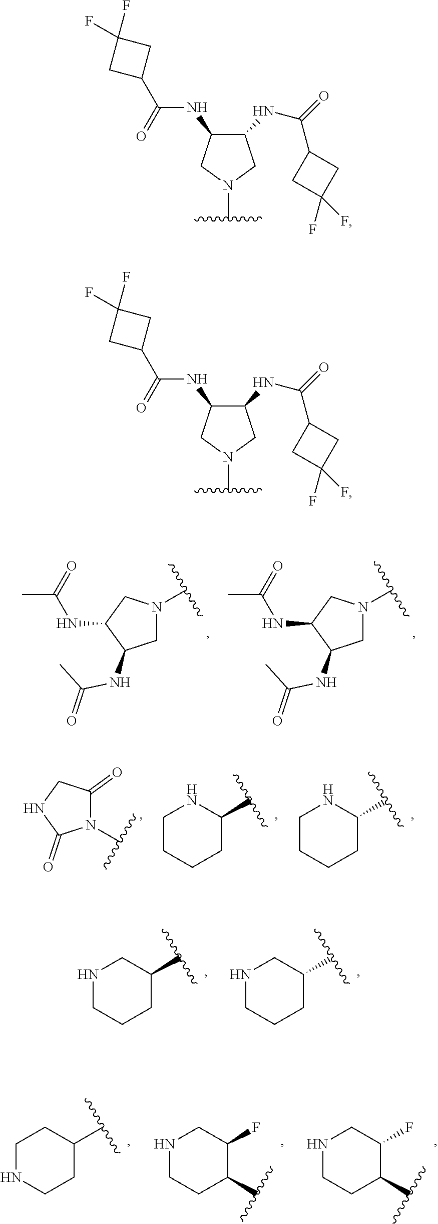

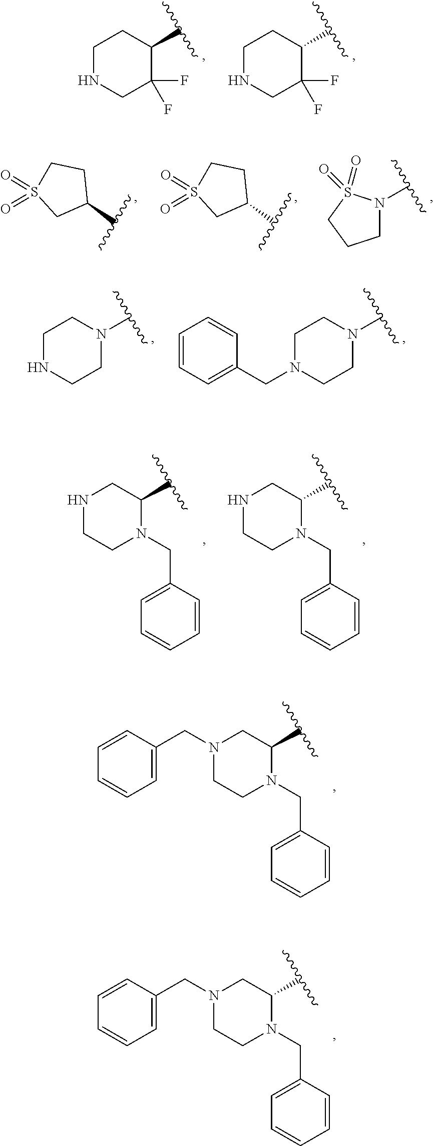

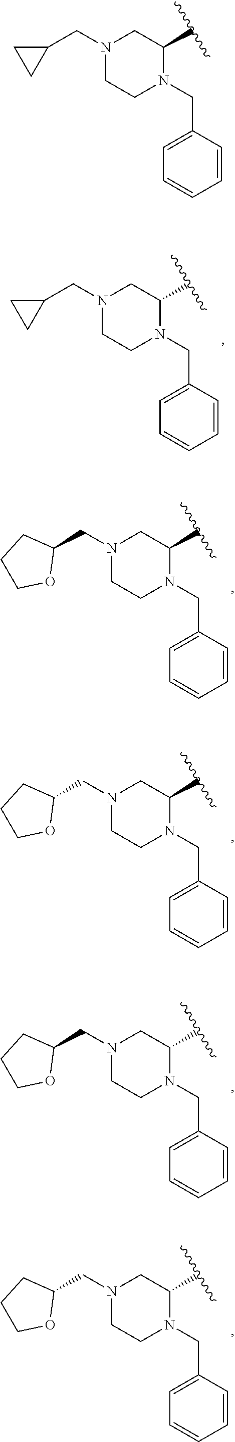 [SMILES: *[C@@H]1CN(CC2CC2)CCN1Cc1ccccc1 ; *[C@H]1CN(CC2CC2)CCN1Cc1ccccc1 ; *[C@@H]1CN(C[C@@H]2CCCO2)CCN1Cc1ccccc1 ; *[C@@H]1CN(C[C@H]2CCCO2)CCN1Cc1ccccc1 ; *[C@H]1CN(C[C@@H]2CCCO2)CCN1Cc1ccccc1 ; *[C@H]1CN(C[C@H]2CCCO2)CCN1Cc1ccccc1]

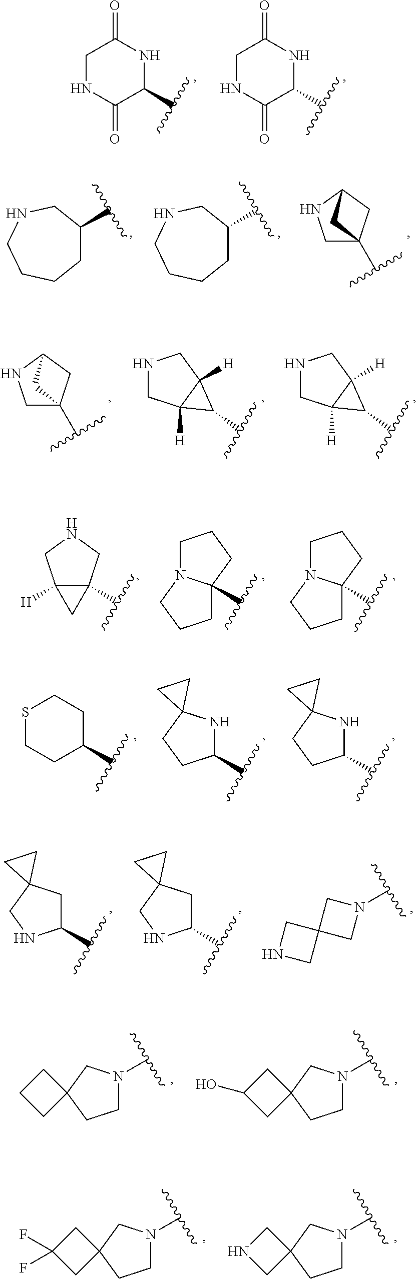

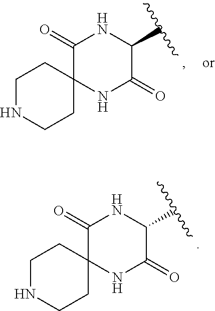 [SMILES: *[C@@H]1NC(=O)C2(CCNCC2)NC1=O ; *[C@H]1NC(=O)C2(CCNCC2)NC1=O]

In some embodiments,

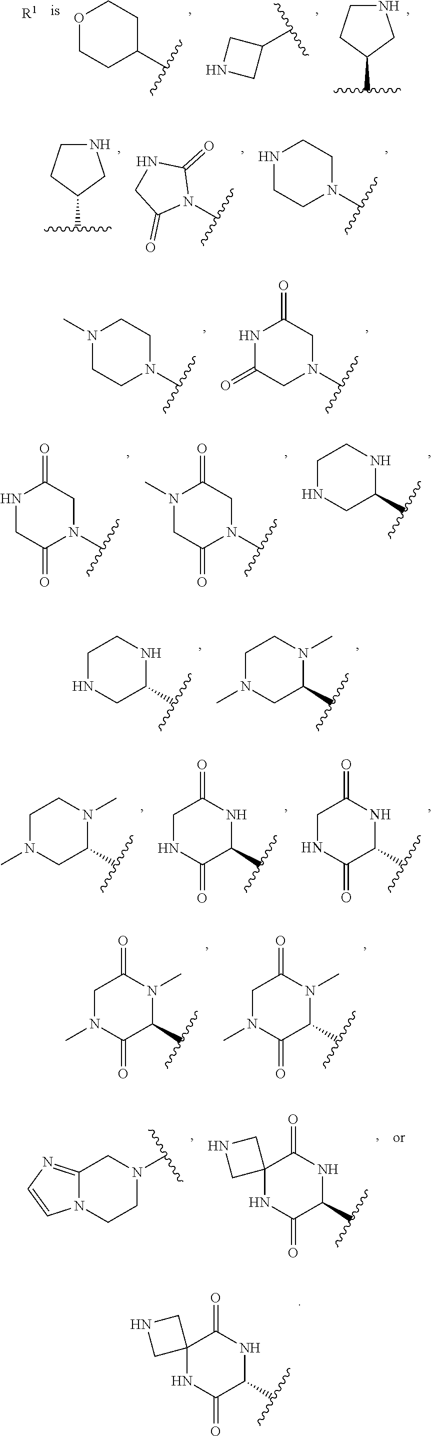

In some embodiments R¹ is

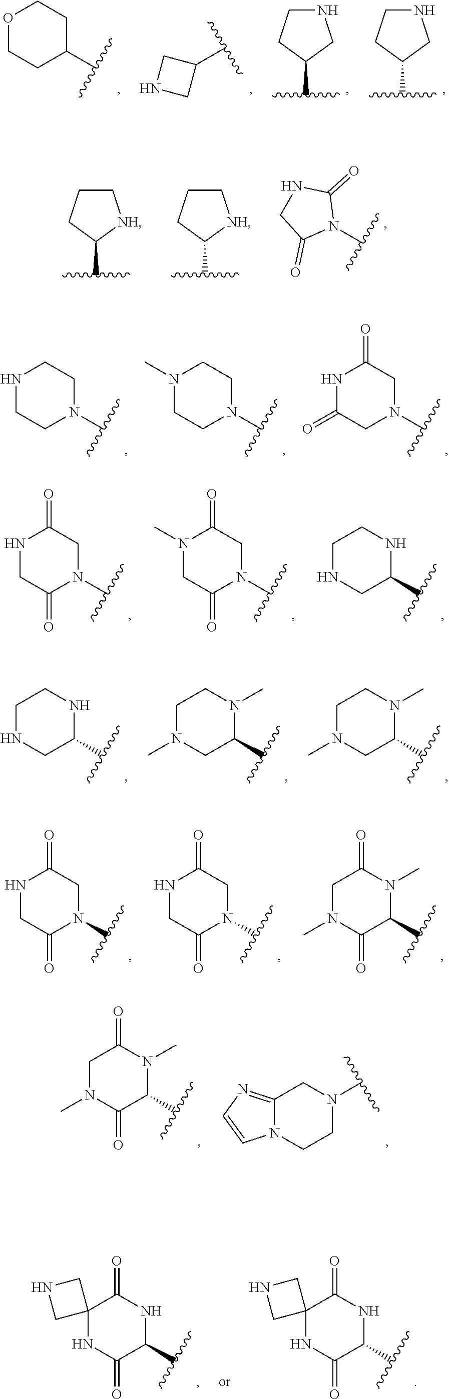

In some embodiments, R¹ is

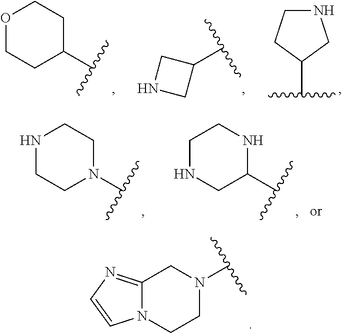

In some embodiments, R¹ is

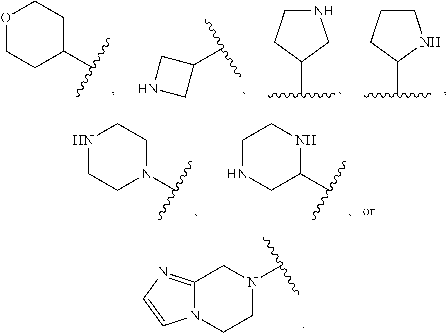

In some embodiments, R¹ is

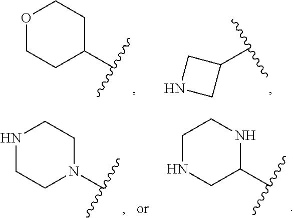

In some embodiments, R¹ is

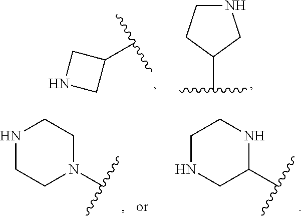

In some embodiments, R¹ is

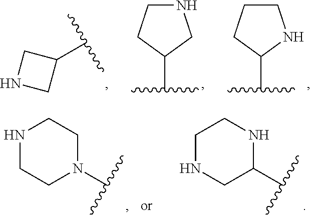

In some embodiments, R¹ is

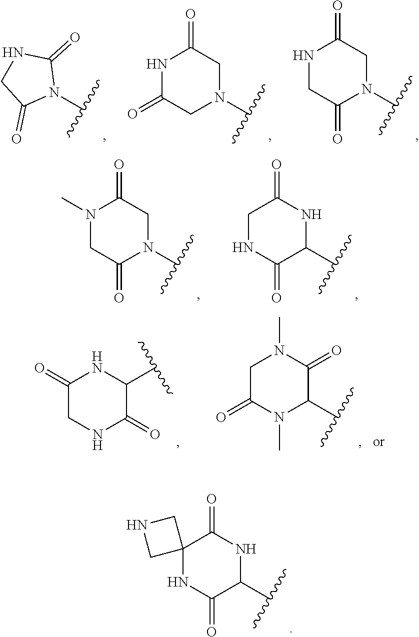

In some embodiments, R¹ is

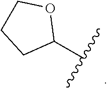 [SMILES: *C1CCCO1]

In some embodiments, R¹ is

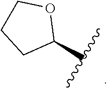 [SMILES: *[C@H]1CCCO1]

In some embodiments, R¹ is

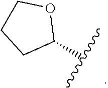 [SMILES: *[C@@H]1CCCO1]

In some embodiments, R¹ is

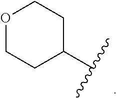 [SMILES: *C1CCOCC1]

In some embodiments, R¹ is

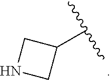 [SMILES: *C1CNC1]

In some embodiments, R¹ is

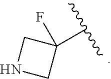 [SMILES: *C1(F)CNC1]

In some embodiments, R¹ is

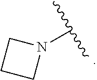 [SMILES: *N1CCC1]

In some embodiments, R¹ is

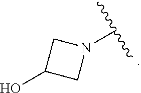 [SMILES: *N1CC(O)C1]

In some embodiments, R¹ is

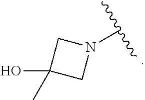 [SMILES: *N1CC(C)(O)C1]

In some embodiments, R¹ is

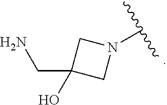 [SMILES: *N1CC(O)(CN)C1]

In some embodiments, R¹ is

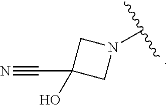 [SMILES: *N1CC(O)(C#N)C1]

In some embodiments, R¹ is

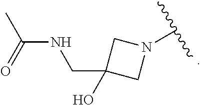 [SMILES: *N1CC(O)(CNC(C)=O)C1]

In some embodiments, R¹ is

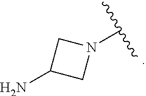 [SMILES: *N1CC(N)C1]

In In some embodiments, R¹ is

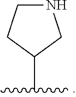 [SMILES: *C1CCNC1]

In some embodiments, R¹ is

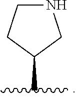 [SMILES: *[C@H]1CCNC1]

In some embodiments, R¹ is

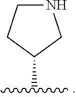 [SMILES: *[C@@H]1CCNC1]

In some embodiments, R¹ is

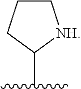 [SMILES: *C1CCCN1]

In some embodiments, R¹ is

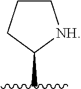 [SMILES: *[C@H]1CCCN1]

In some embodiments, R¹ is

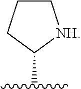 [SMILES: *[C@@H]1CCCN1]

In some embodiments, R¹ is

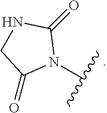 [SMILES: *N1C(=O)CNC1=O]

In some embodiments, R¹ is

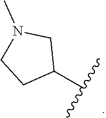 [SMILES: *C1CCN(C)C1]

In some embodiments, R¹ is

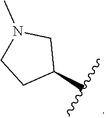 [SMILES: *[C@H]1CCN(C)C1]

In some embodiments, R¹ is

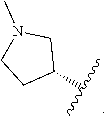 [SMILES: *[C@@H]1CCN(C)C1]

In some embodiments, R¹ is

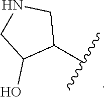 [SMILES: *C1CNCC1O]

In some embodiments, R¹ is

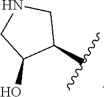 [SMILES: *[C@@H]1CNC[C@@H]1O]

In some embodiments, R¹ is

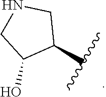 [SMILES: *[C@@H]1CNC[C@H]1O]

In some embodiments, R¹ is

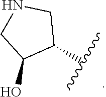 [SMILES: *[C@H]1CNC[C@@H]1O]

In some embodiments, R¹ is

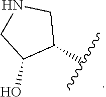 [SMILES: *[C@H]1CNC[C@H]1O]

In some embodiments, R¹ is

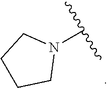 [SMILES: *N1CCCC1]

In some embodiments, R¹ is

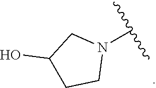 [SMILES: *N1CCC(O)C1]

In some embodiments, R¹ is

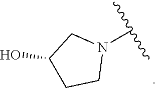 [SMILES: *N1CC[C@H](O)C1]

In some embodiments, R¹ is

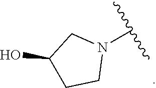 [SMILES: *N1CC[C@@H](O)C1]

In some embodiments, R¹ is

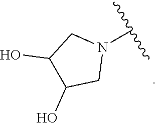 [SMILES: *N1CC(O)C(O)C1]

In some embodiments, R¹ is

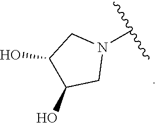 [SMILES: *N1C[C@@H](O)[C@H](O)C1]

In some embodiments, R¹ is

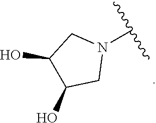 [SMILES: *N1C[C@@H](O)[C@@H](O)C1]

In some embodiments, R¹ is

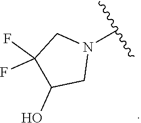 [SMILES: *N1CC(O)C(F)(F)C1]

In some embodiments, R¹ is

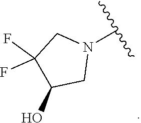 [SMILES: *N1C[C@@H](O)C(F)(F)C1]

In some embodiments, R¹ is

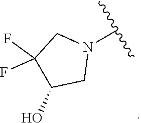 [SMILES: *N1C[C@H](O)C(F)(F)C1]

In some embodiments, R¹ is

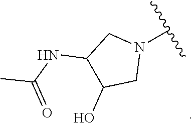 [SMILES: *N1CC(O)C(NC(C)=O)C1]

In some embodiments, R¹ is

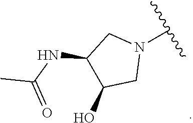 [SMILES: *N1C[C@@H](O)[C@@H](NC(C)=O)C1]

In some embodiments, R¹ is

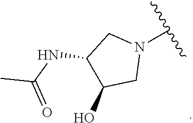 [SMILES: *N1C[C@@H](O)[C@H](NC(C)=O)C1]

In some embodiments, R¹ is

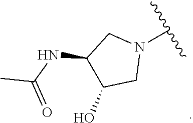 [SMILES: *N1C[C@H](NC(C)=O)[C@@H](O)C1]

In some embodiments, R¹ is

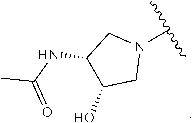 [SMILES: *N1C[C@H](O)[C@H](NC(C)=O)C1]

In some embodiments, R¹ is

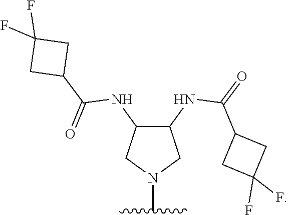 [SMILES: *N1CC(NC(=O)C2CC(F)(F)C2)C(NC(=O)C2CC(F)(F)C2)C1]

In some embodiments, R¹ is

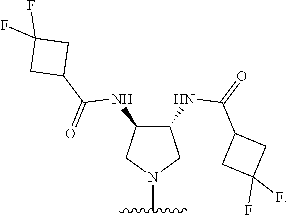 [SMILES: *N1C[C@@H](NC(=O)C2CC(F)(F)C2)[C@H](NC(=O)C2CC(F)(F)C2)C1]

In some embodiments, R¹ is

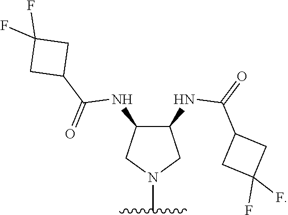 [SMILES: *N1C[C@H](NC(=O)C2CC(F)(F)C2)[C@H](NC(=O)C2CC(F)(F)C2)C1]

In some embodiments, R¹ is

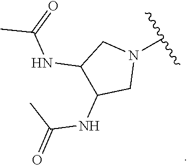 [SMILES: *N1CC(NC(C)=O)C(NC(C)=O)C1]

In some embodiments, R¹ is

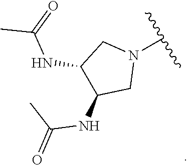 [SMILES: *N1C[C@@H](NC(C)=O)[C@H](NC(C)=O)C1]

In some embodiments, R¹ is

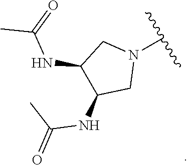 [SMILES: *N1C[C@H](NC(C)=O)[C@H](NC(C)=O)C1]

In some embodiments, R¹ is

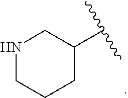 [SMILES: *C1CCCNC1]

In some embodiments, R¹ is

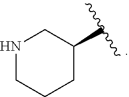 [SMILES: *[C@H]1CCCNC1]

In some embodiments, R¹ is

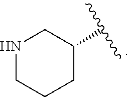 [SMILES: *[C@@H]1CCCNC1]

In some embodiments, R¹ is

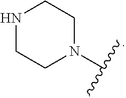 [SMILES: *N1CCNCC1]

In some embodiments, R¹ is

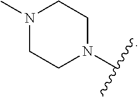 [SMILES: *N1CCN(C)CC1]

In some embodiments, R¹ is

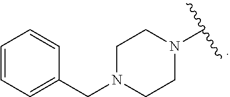 [SMILES: *N1CCN(Cc2ccccc2)CC1]

In some embodiments, R¹ is

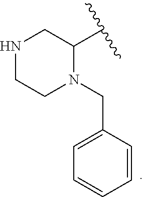 [SMILES: *C1CNCCN1Cc1ccccc1]

In some embodiments, R¹ is

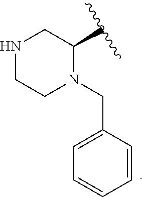 [SMILES: *[C@@H]1CNCCN1Cc1ccccc1]

In some embodiments, R¹ is

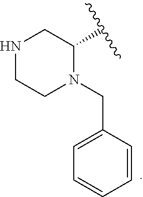 [SMILES: *[C@H]1CNCCN1Cc1ccccc1]

In some embodiments, R¹ is

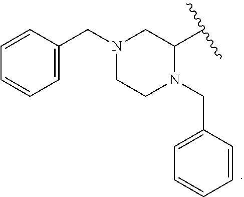 [SMILES: *C1CN(Cc2ccccc2)CCN1Cc1ccccc1]

In some embodiments, R¹ is

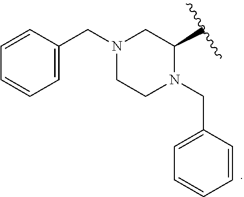 [SMILES: *[C@@H]1CN(Cc2ccccc2)CCN1Cc1ccccc1]

In some embodiments, R¹ is

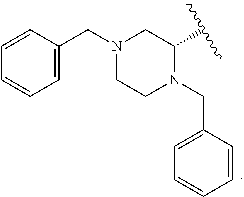 [SMILES: *[C@H]1CN(Cc2ccccc2)CCN1Cc1ccccc1]

In some embodiments, R¹ is

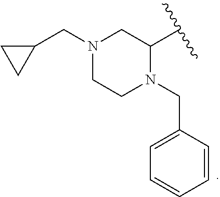 [SMILES: *C1CN(CC2CC2)CCN1Cc1ccccc1]

In some embodiments, R¹ is

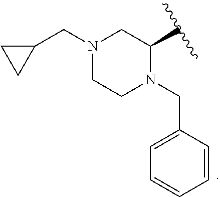 [SMILES: *[C@@H]1CN(CC2CC2)CCN1Cc1ccccc1]

In some embodiments, R¹ is

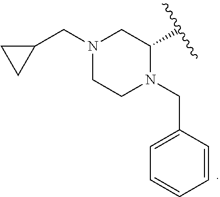 [SMILES: *[C@H]1CN(CC2CC2)CCN1Cc1ccccc1]

In some embodiments, R¹ is

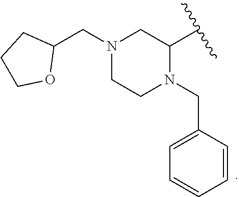 [SMILES: *C1CN(CC2CCCO2)CCN1Cc1ccccc1]

In some embodiments, R¹ is

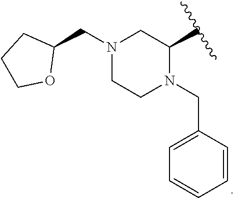 [SMILES: *[C@@H]1CN(C[C@@H]2CCCO2)CCN1Cc1ccccc1]

In some embodiments, R¹ is

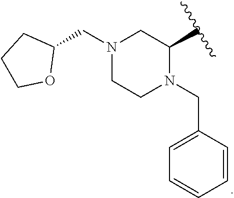 [SMILES: *[C@@H]1CN(C[C@H]2CCCO2)CCN1Cc1ccccc1]

In some embodiments, R¹ is

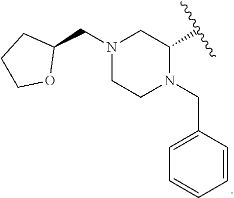 [SMILES: *[C@H]1CN(C[C@@H]2CCCO2)CCN1Cc1ccccc1]

In some embodiments, R¹ is

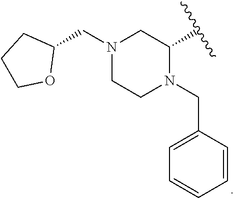 [SMILES: *[C@H]1CN(C[C@H]2CCCO2)CCN1Cc1ccccc1]

In some embodiments, R¹ is

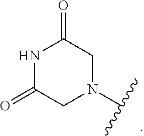 [SMILES: *N1CC(=O)NC(=O)C1]

In some embodiments, R¹ is

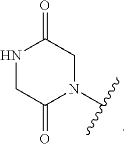 [SMILES: *N1CC(=O)NCC1=O]

In some embodiments, R¹ is

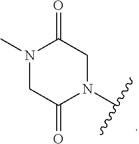 [SMILES: *N1CC(=O)N(C)CC1=O]

In some embodiments, R¹ is

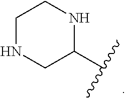 [SMILES: *C1CNCCN1]

In some embodiments, R¹ is

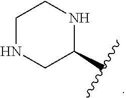 [SMILES: *[C@H]1CNCCN1]

In some embodiments, R¹ is

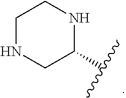 [SMILES: *[C@@H]1CNCCN1]

In some embodiments, R¹ is

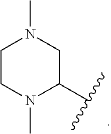 [SMILES: *C1CN(C)CCN1C]

In some embodiments, R¹ is

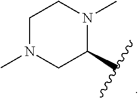

In some embodiments, R¹ is

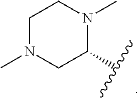

In some embodiments, R¹ is

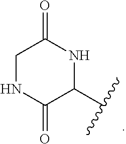 [SMILES: *C1NC(=O)CNC1=O]

In some embodiments, R¹ is

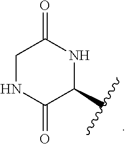 [SMILES: *[C@@H]1NC(=O)CNC1=O]

In some embodiments, R¹ is

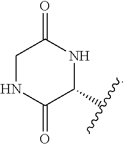 [SMILES: *[C@H]1NC(=O)CNC1=O]

In some embodiments, R¹ is

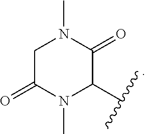 [SMILES: *C1C(=O)N(C)CC(=O)N1C]

In some embodiments, R¹ is

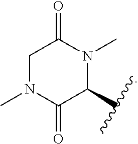

In some embodiments, R¹ is

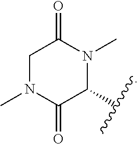

In some embodiments, R¹ is

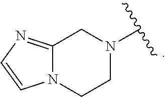 [SMILES: *N1CCn2ccnc2C1]

In some embodiments, R¹ is

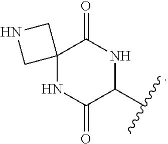 [SMILES: *C1NC(=O)C2(CNC2)NC1=O]

In some embodiments, R¹ is

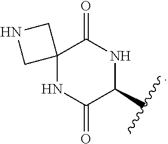 [SMILES: *[C@@H]1NC(=O)C2(CNC2)NC1=O]

In some embodiments, R¹ is

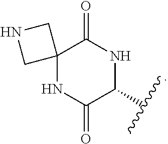 [SMILES: *[C@H]1NC(=O)C2(CNC2)NC1=O]

In some embodiments, R¹ is

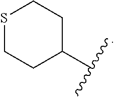 [SMILES: *C1CCSCC1]

In some embodiments, R¹ is

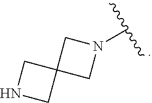 [SMILES: *N1CC2(CNC2)C1]

In some embodiments, R¹ is

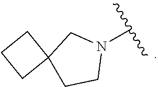 [SMILES: *N1CCC2(CCC2)C1]

In some embodiments, R¹ is

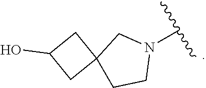 [SMILES: *N1CCC2(CC(O)C2)C1]

In some embodiments, R¹ is

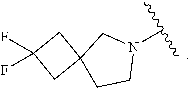 [SMILES: *N1CCC2(C1)CC(F)(F)C2]

In some embodiments, R¹ is

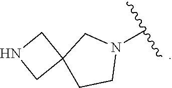 [SMILES: *N1CCC2(CNC2)C1]

In some embodiments, R¹ is

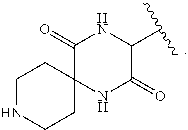 [SMILES: *C1NC(=O)C2(CCNCC2)NC1=O]

In some embodiments, R¹ is

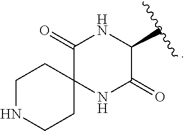 [SMILES: *[C@@H]1NC(=O)C2(CCNCC2)NC1=O]

In some embodiments, R¹ is

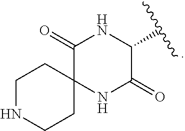 [SMILES: *[C@H]1NC(=O)C2(CCNCC2)NC1=O]

In some embodiments, R¹ is optionally substituted aryl or optionally substituted heteroaryl. In some embodiments, R¹ is optionally substituted aryl. In some embodiments, R¹ is optionally substituted C₆₋₁₄ aryl. In some embodiments, R¹ is optionally substituted phenyl. In some embodiments, R¹ is unsubstituted phenyl. In some embodiments, R¹ is substituted phenyl. In some embodiments, R¹ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂. In some embodiments, R¹ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —OR^{1a}, and/or —N(R^{1a})₂. In some embodiments, R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂. In some embodiments, R¹ is phenyl optionally substituted with halogen, optionally substituted alkyl, —OR^{1a}, and/or —N(R^{1a})₂. In some embodiments, R¹ is phenyl substituted with halogen, optionally substituted alkyl, —OR^{1a}, and/or —N(R^{1a})₂. In some embodiments, R¹ is optionally substituted naphthyl. In some embodiments, R¹ is substituted naphthyl. In some embodiments, R¹ is unsubstituted naphthyl.

In some embodiments, R¹ is phenyl substituted with halogen. In some embodiments, R¹ is phenyl substituted with —F, —Cl, —Br, or —I. In some embodiments, R¹ is phenyl substituted with —F, —Cl, or —Br. In some embodiments, R¹ is phenyl substituted with —F. In some embodiments, R¹ is phenyl substituted with —Cl. In some embodiments, R¹ is phenyl substituted with —Br. In some embodiments, R¹ is phenyl substituted with —F and —Cl.

In some embodiments, R¹ is phenyl substituted with optionally substituted alkyl. In some embodiments, R¹ is phenyl substituted with optionally substituted C₁₋₁₂ alkyl. In some embodiments, R¹ is phenyl substituted with optionally substituted C₁₋₆ alkyl. In some embodiments, R¹ is phenyl substituted with optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R¹ is phenyl substituted with substituted C₁₋₆ alkyl. In some embodiments, R¹ is phenyl substituted with substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R¹ is phenyl substituted with unsubstituted C₁₋₆ alkyl. In some embodiments, R¹ is phenyl substituted with methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R¹ is phenyl substituted with isobutyl.

In some embodiments, R¹ is phenyl substituted with —OR^{1a}. In some embodiments, R¹ is phenyl substituted with —O(optionally substituted alkyl). In some embodiments, R¹ is phenyl substituted with —O(optionally substituted C₁₋₆ alkyl). In some embodiments, R¹ is phenyl substituted with —O(optionally substituted alkyl optionally substituted with halogen). In some embodiments, R¹ is phenyl substituted with —O(optionally substituted C₁₋₆ alkyl optionally substituted with halogen). In some embodiments, R¹ is phenyl substituted with —O(optionally substituted alkyl optionally substituted with —F). In some embodiments, R¹ is phenyl substituted with —O(optionally substituted C₁₋₆ alkyl optionally substituted with —F). In some embodiments, R¹ is phenyl substituted with —OCH₂F, —OCHF₂, or —OCF₃. In some embodiments, R¹ is phenyl substituted with —OCF₃.

In some embodiments, R¹ is phenyl substituted with —N(R^{1a})₂. In some embodiments, R¹ is phenyl substituted with —N(optionally substituted alkyl)₂. In some embodiments, R¹ is phenyl substituted with —N(optionally substituted C₁₋₆ alkyl)₂. In some embodiments, R¹ is phenyl substituted with —N(R^{1a})₂, wherein two instances of R^{1a} are joined together with their intervening atom to form an optionally substituted heterocyclic ring or optionally substituted heteroaryl ring. In some embodiments, R¹ is phenyl substituted with —N(R^{1a})₂, wherein two instances of R^{1a} are joined together with their intervening atom to form an optionally substituted heterocyclic ring. In some embodiments, R¹ is phenyl substituted with —N(R^{1a})₂, wherein two instances of R^{1a} are joined together with their intervening atom to form an optionally substituted 3-10 membered heterocyclic ring. In some embodiments, R¹ is phenyl substituted with —N(R^{1a})₂, wherein two instances of R^{1a} are joined together with their intervening atom to form an optionally substituted 3-7 membered heterocyclic ring. In some embodiments, R¹ is phenyl substituted with —N(R^{1a})₂, wherein two instances of R^{1a} are joined together with their intervening atom to form an optionally substituted 6 membered heterocyclic ring. In some embodiments, R¹ is phenyl substituted with —N(R^{1a})₂, wherein two instances of R^{1a} are joined together with their intervening atom to form an optionally substituted morpholinyl. In some embodiments, R¹ is phenyl substituted with optionally substituted morpholinyl. In some embodiments, R¹ is phenyl substituted with substituted morpholinyl. In some embodiments, R¹ is phenyl substituted with unsubstituted morpholinyl.

In some embodiments, R¹ is of formula:

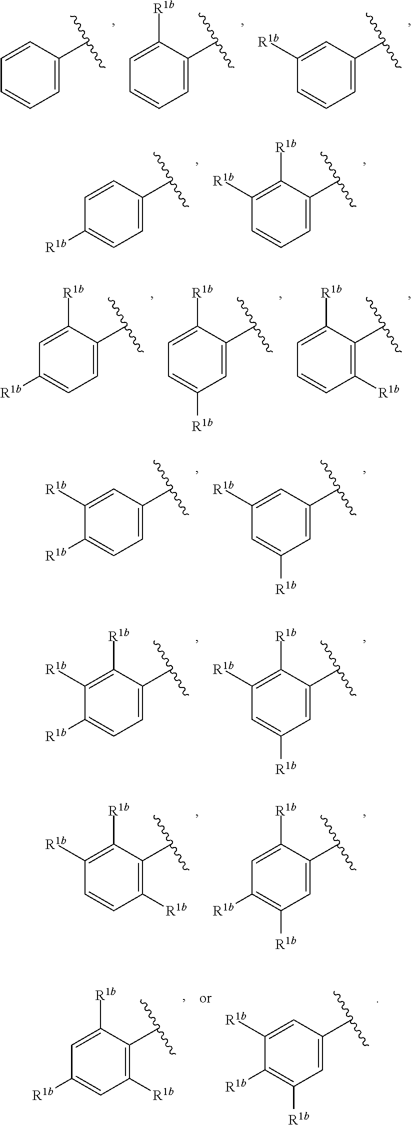

wherein each instance of R^{1b} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})OR^{A}₂, —OSi(OR^{A})₃, or —B(OR^{A})₂. In some embodiments, R¹ is of formula:

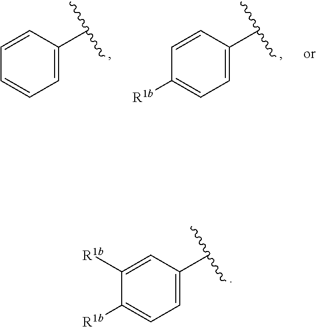

In some embodiments, R¹ is

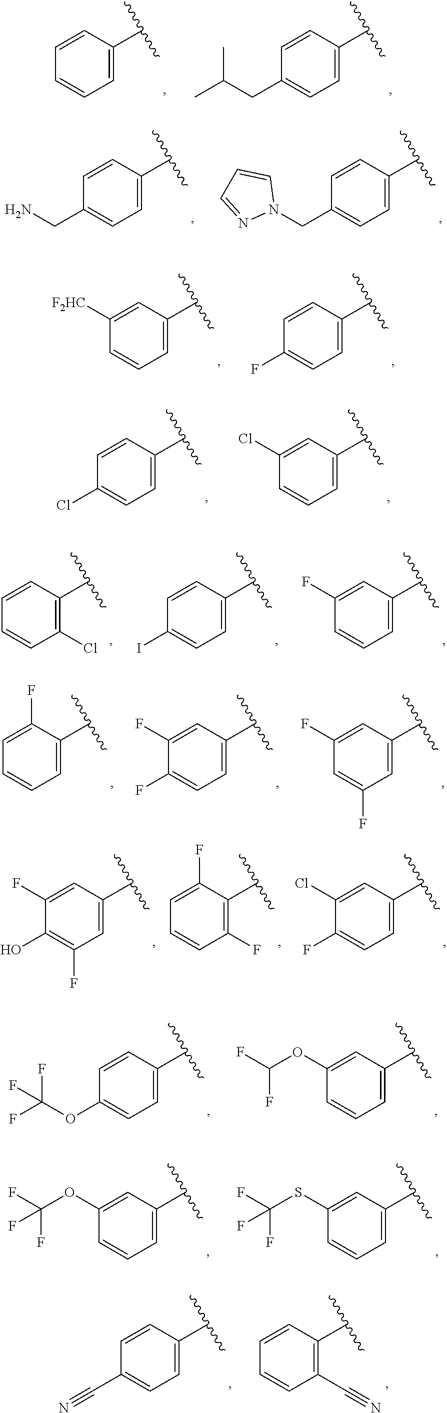

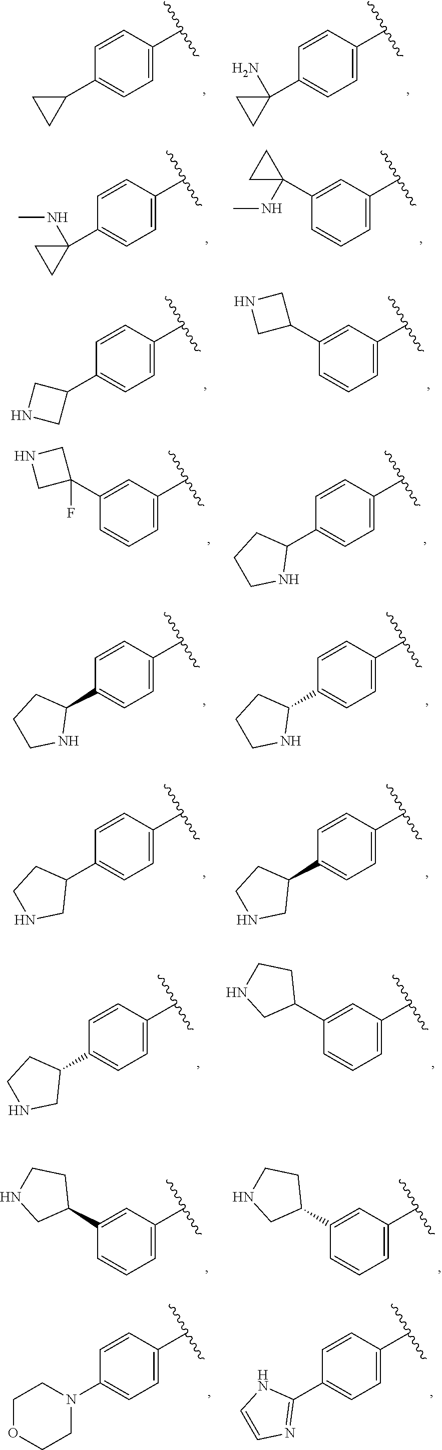

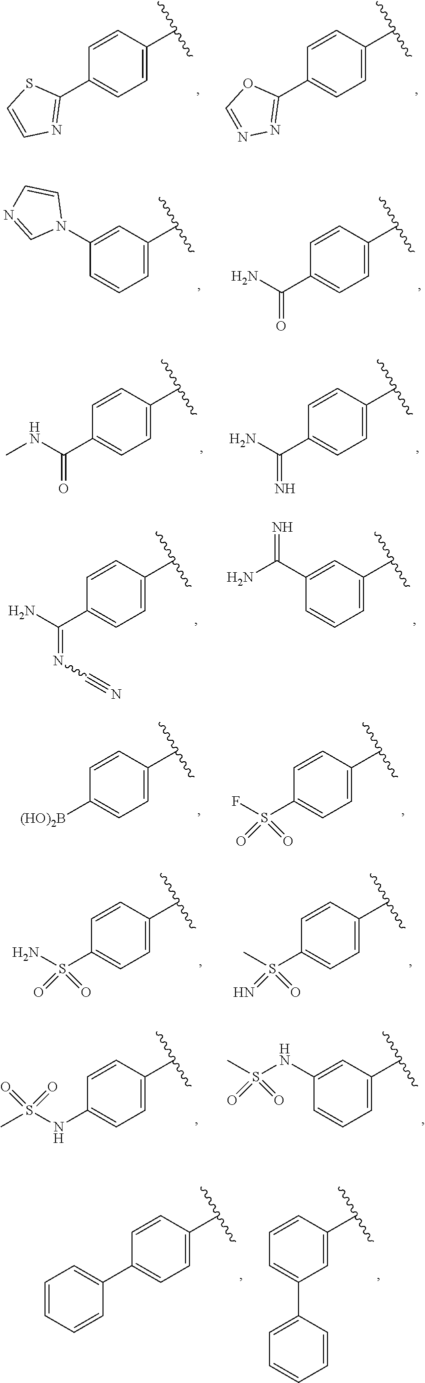

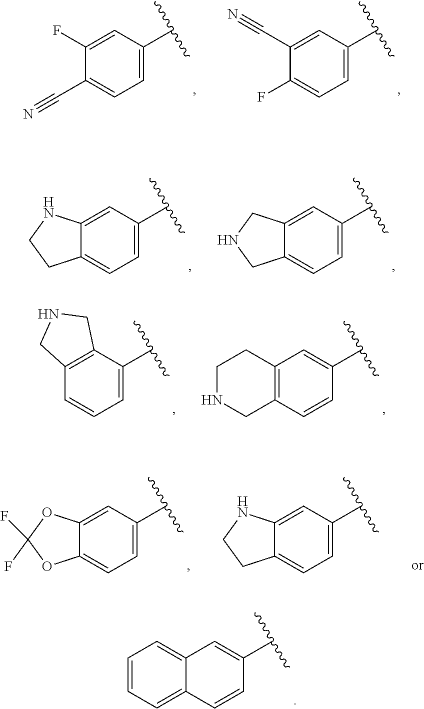

In some embodiments, R¹ is

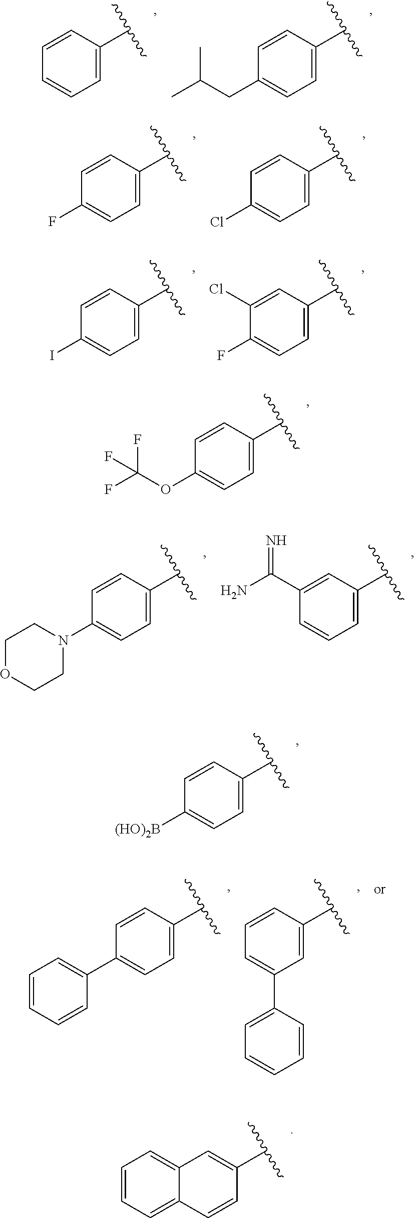

In some embodiments, R¹ is

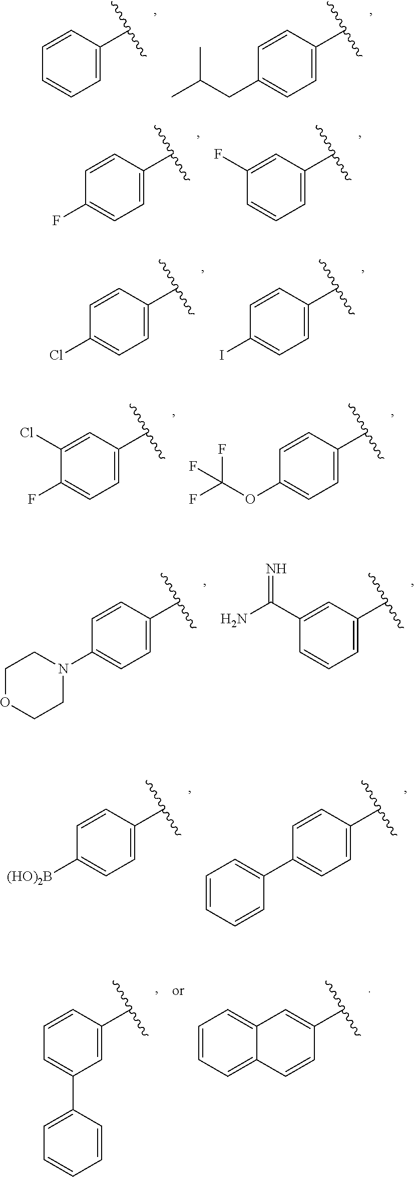

In some embodiments, R¹ is

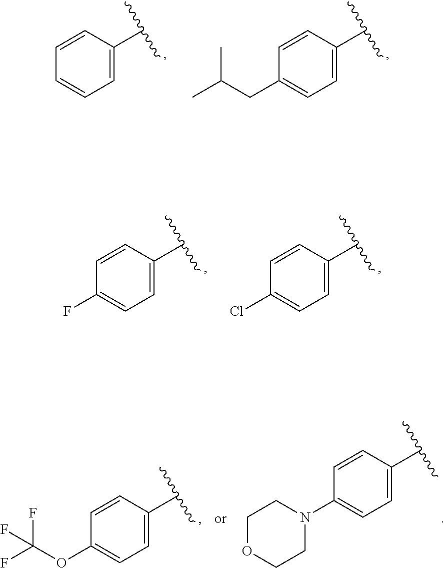 [SMILES: *c1ccccc1 ; *c1ccc(CC(C)C)cc1 ; *c1ccc(F)cc1 ; *c1ccc(Cl)cc1 ; *c1ccc(OC(F)(F)F)cc1 ; *c1ccc(N2CCOCC2)cc1]

In some embodiments, R¹ is

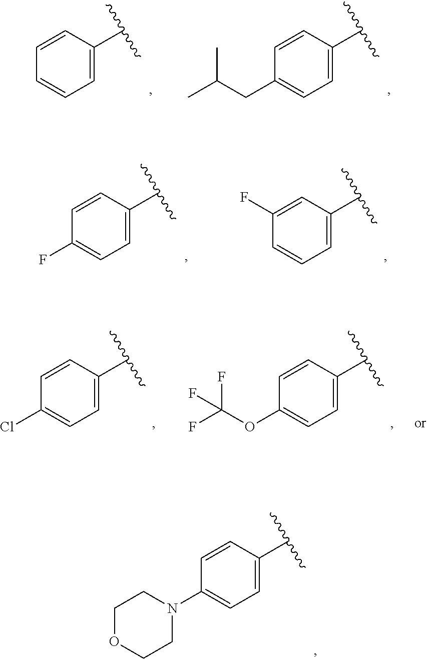

In some embodiments, R¹ is

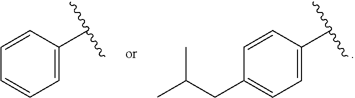 [SMILES: *c1ccccc1 ; *c1ccc(CC(C)C)cc1]

In some embodiments, R¹ is

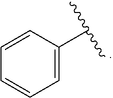 [SMILES: *c1ccccc1]

In some embodiments, R¹ is

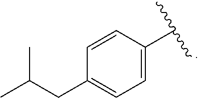 [SMILES: *c1ccc(CC(C)C)cc1]

In some embodiments, R¹ is

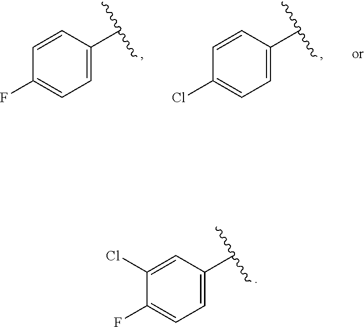 [SMILES: *c1ccc(F)cc1 ; *c1ccc(Cl)cc1 ; *c1ccc(F)c(Cl)c1]

In some embodiments, R¹ is

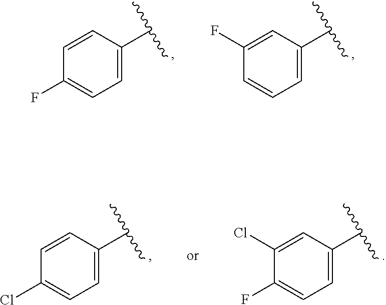 [SMILES: *c1ccc(F)cc1 ; *c1cccc(F)c1 ; *c1ccc(Cl)cc1 ; *c1ccc(F)c(Cl)c1]

In some embodiments, R¹ is

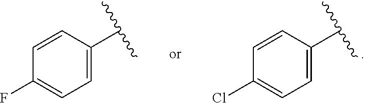 [SMILES: *c1ccc(F)cc1 ; *c1ccc(Cl)cc1]

In some embodiments, R¹ is

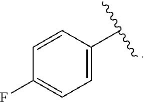 [SMILES: *c1ccc(F)cc1]

In some embodiments, R¹ is

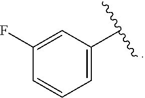 [SMILES: *c1cccc(F)c1]

In some embodiments, R¹ is

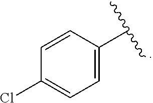 [SMILES: *c1ccc(Cl)cc1]

In some embodiments, R¹ is

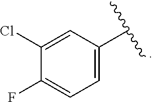 [SMILES: *c1ccc(F)c(Cl)c1]

In some embodiments, R¹ is

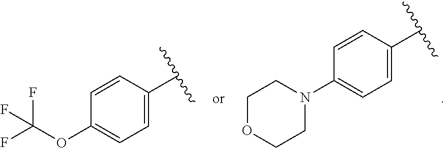 [SMILES: *c1ccc(OC(F)(F)F)cc1 ; *c1ccc(N2CCOCC2)cc1]

In some embodiments, R¹ is

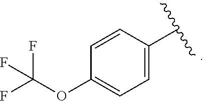 [SMILES: *c1ccc(OC(F)(F)F)cc1]

In some embodiments, R¹ is

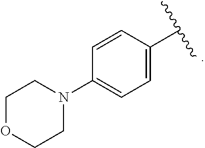 [SMILES: *c1ccc(N2CCOCC2)cc1]

In some embodiments, R¹ is

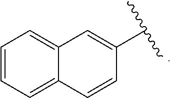 [SMILES: *c1ccc2ccccc2c1]

In some embodiments, R¹ is optionally substituted heteroaryl. In some embodiments, R¹ is optionally substituted 4-10 membered heteroaryl. In some embodiments, R¹ is optionally substituted 5-6 membered heteroaryl. In some embodiments, R¹ is optionally substituted monocyclic 5-6 membered heteroaryl. In some embodiments, R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted benzotriazolyl, optionally substituted purinyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl. In some embodiments, R¹ is substituted pyridyl, substituted pyrazinyl, substituted pyrimidinyl, substituted pyridazinyl, substituted pyrazolyl, substituted imidazolyl, substituted triazolyl, substituted tetrazolyl, substituted benzimidazolyl, substituted benzotriazolyl, substituted purinyl, substituted thiophenyl, substituted benzothiophenyl, substituted oxazolyl, substituted isoxazolyl, substituted oxadiazolyl, substituted benzooxadiazolyl, substituted thiazolyl, substituted thiadiazolyl, or substituted benzothiadiazolyl. In some embodiments, R¹ is unsubstituted pyridyl, unsubstituted pyrazinyl, unsubstituted pyrimidinyl, unsubstituted pyridazinyl, unsubstituted pyrazolyl, unsubstituted imidazolyl, unsubstituted triazolyl, unsubstituted tetrazolyl, unsubstituted benzimidazolyl, unsubstituted benzotriazolyl, unsubstituted purinyl, unsubstituted thiophenyl, unsubstituted benzothiophenyl, unsubstituted oxazolyl, unsubstituted isoxazolyl, unsubstituted oxadiazolyl, unsubstituted benzooxadiazolyl, unsubstituted thiazolyl, unsubstituted thiadiazolyl, or unsubstituted benzothiadiazolyl. In some embodiments, R¹ is optionally substituted pyridyl. In some embodiments, R¹ is optionally substituted pyrazinyl. In some embodiments, R¹ is optionally substituted pyrimidinyl. In some embodiments, R¹ is optionally substituted pyridazinyl. In some embodiments, R¹ is optionally substituted pyrazolyl. In some embodiments, R¹ is optionally substituted imidazolyl. In some embodiments, R¹ is optionally substituted triazolyl. In some embodiments, R¹ is optionally substituted tetrazolyl. In some embodiments, R¹ is optionally substituted benzimidazolyl. In some embodiments, R¹ is optionally substituted benzotriazolyl. In some embodiments, R¹ is optionally substituted purinyl. In some embodiments, R¹ is optionally substituted thiophenyl. In some embodiments, R¹ is optionally substituted benzothiophenyl. In some embodiments, R¹ is optionally substituted oxazolyl. In some embodiments, R¹ is optionally substituted isoxazolyl. In some embodiments, R¹ is optionally substituted oxadiazolyl. In some embodiments, R¹ is optionally substituted benzooxadiazolyl. In some embodiments, R¹ is optionally substituted thiazolyl. In some embodiments, R¹ is optionally substituted thiadiazolyl. In some embodiments, R¹ is optionally substituted benzothiadiazolyl.

In some embodiments, R¹ is optionally substituted pyridyl, optionally substituted pyrazinyl, optionally substituted pyrimidinyl, optionally substituted imidazolyl, optionally substituted triazolyl, optionally substituted tetrazolyl, optionally substituted benzotriazolyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl. In some embodiments, R¹ is substituted pyridyl, substituted pyrazinyl, substituted pyrimidinyl, substituted imidazolyl, substituted triazolyl, substituted tetrazolyl, substituted benzotriazolyl, substituted thiophenyl, substituted benzothiophenyl, substituted oxazolyl, substituted isoxazolyl, substituted oxadiazolyl, substituted benzooxadiazolyl, substituted thiazolyl, substituted thiadiazolyl, or substituted benzothiadiazolyl. In some embodiments, R¹ is substituted pyridyl. In some embodiments, R¹ is substituted pyrazinyl. In some embodiments, R¹ is substituted pyrimidinyl. In some embodiments, R¹ is substituted imidazolyl. In some embodiments, R¹ is substituted triazolyl. In some embodiments, R¹ is substituted tetrazolyl. In some embodiments, R¹ is substituted benzotriazolyl. In some embodiments, R¹ is substituted thiophenyl. In some embodiments, R¹ is substituted benzothiophenyl. In some embodiments, R¹ is substituted oxazolyl. In some embodiments, R¹ is substituted isoxazolyl. In some embodiments, R¹ is substituted oxadiazolyl. In some embodiments, R¹ is substituted benzooxadiazolyl. In some embodiments, R¹ is substituted thiazolyl. In some embodiments, R¹ is substituted thiadiazolyl. In some embodiments, R¹ is substituted benzothiadiazolyl. In some embodiments, R¹ is unsubstituted pyridyl, unsubstituted pyrazinyl, unsubstituted pyrimidinyl, unsubstituted imidazolyl, unsubstituted triazolyl, unsubstituted tetrazolyl, unsubstituted benzotriazolyl, unsubstituted thiophenyl, unsubstituted benzothiophenyl, unsubstituted oxazolyl, unsubstituted isoxazolyl, unsubstituted oxadiazolyl, unsubstituted benzooxadiazolyl, unsubstituted thiazolyl, unsubstituted thiadiazolyl, or unsubstituted benzothiadiazolyl. In some embodiments, R¹ is unsubstituted pyridyl. In some embodiments, R¹ is unsubstituted pyrazinyl. In some embodiments, R¹ is unsubstituted pyrimidinyl. In some embodiments, R¹ is unsubstituted imidazolyl. In some embodiments, R¹ is unsubstituted triazolyl. In some embodiments, R¹ is unsubstituted tetrazolyl. In some embodiments, R¹ is unsubstituted benzotriazolyl. In some embodiments, R¹ is unsubstituted thiophenyl. In some embodiments, R¹ is unsubstituted benzothiophenyl. In some embodiments, R¹ is unsubstituted oxazolyl. In some embodiments, R¹ is unsubstituted isoxazolyl. In some embodiments, R¹ is unsubstituted oxadiazolyl. In some embodiments, R¹ is unsubstituted benzooxadiazolyl. In some embodiments, R¹ is unsubstituted thiazolyl. In some embodiments, R¹ is unsubstituted thiadiazolyl. In some embodiments, R¹ is unsubstituted benzothiadiazolyl.

In some embodiments, R¹ is of formula:

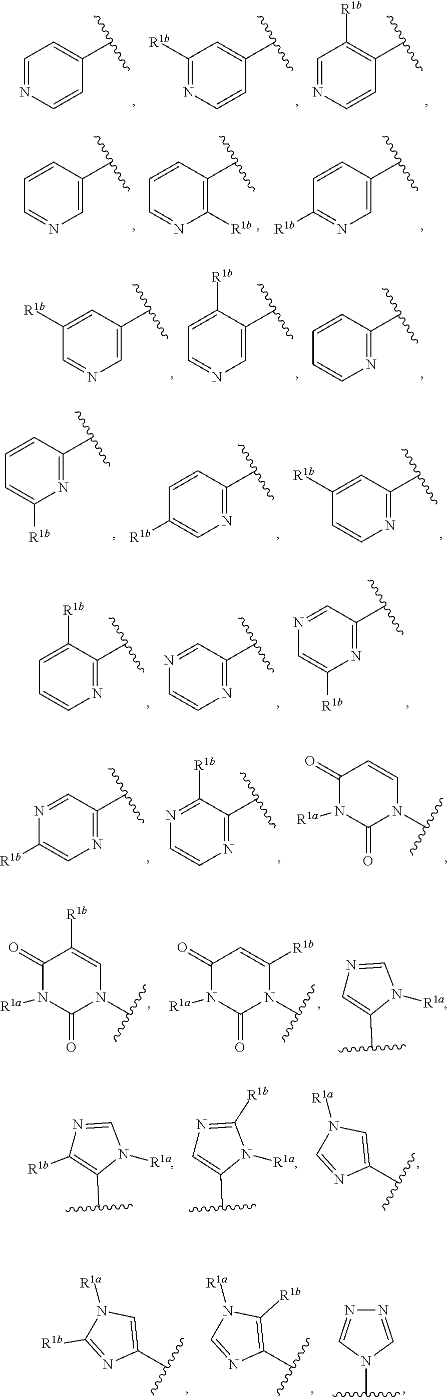

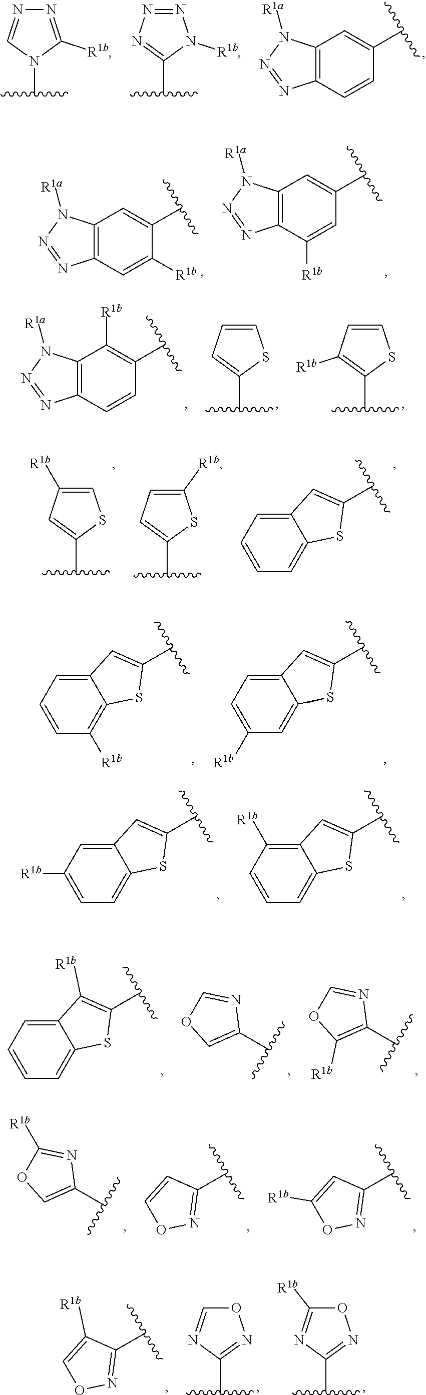

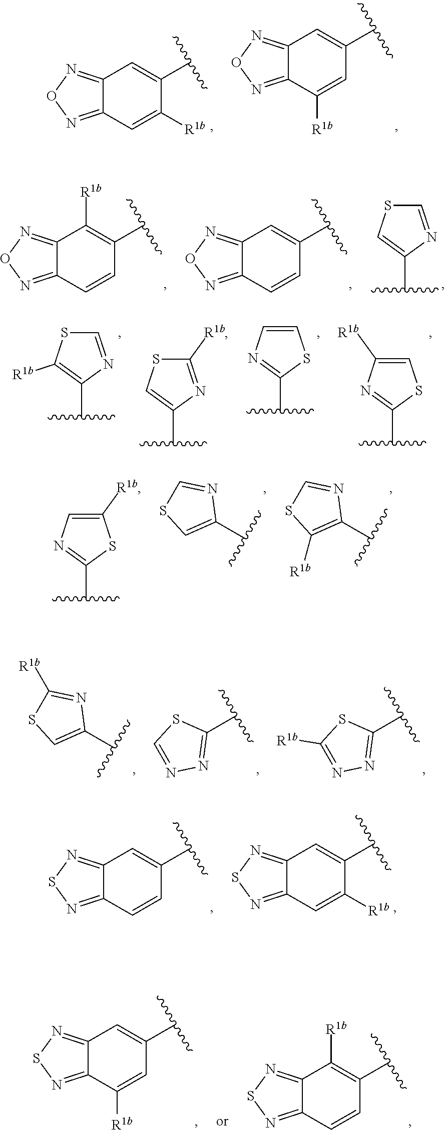

wherein:
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group; and
each instance of R^{1b} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In some embodiments, R¹ is

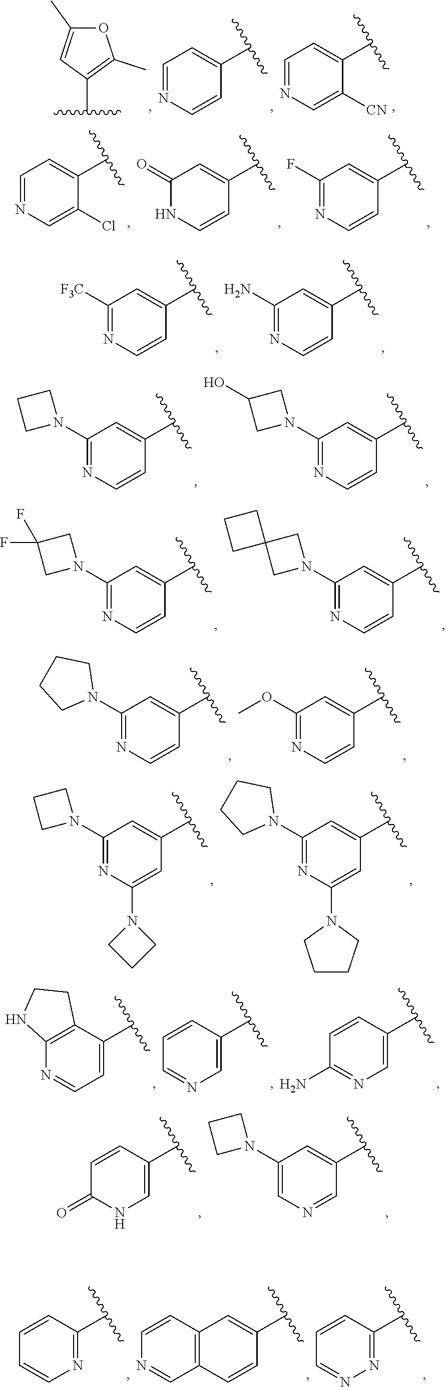

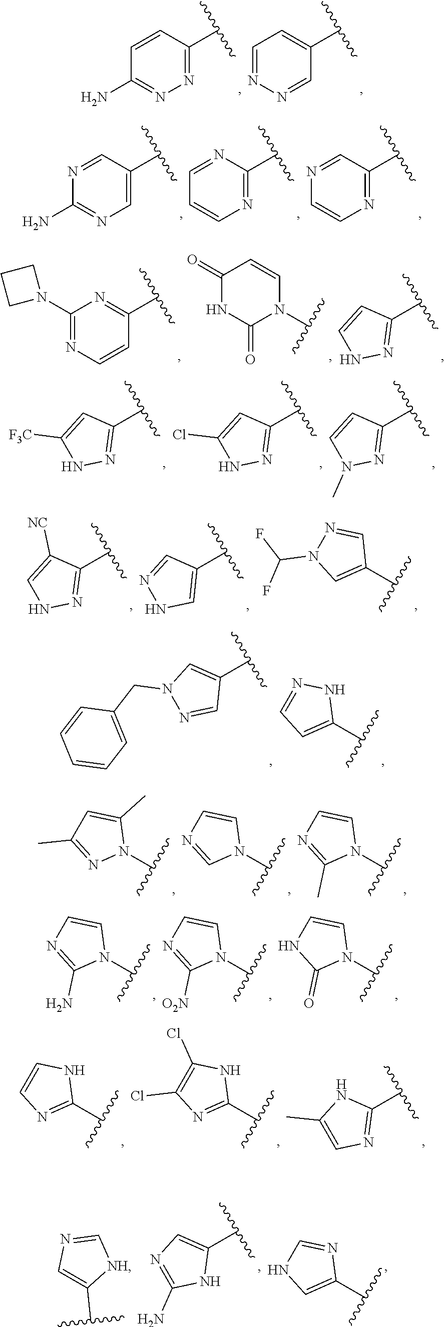

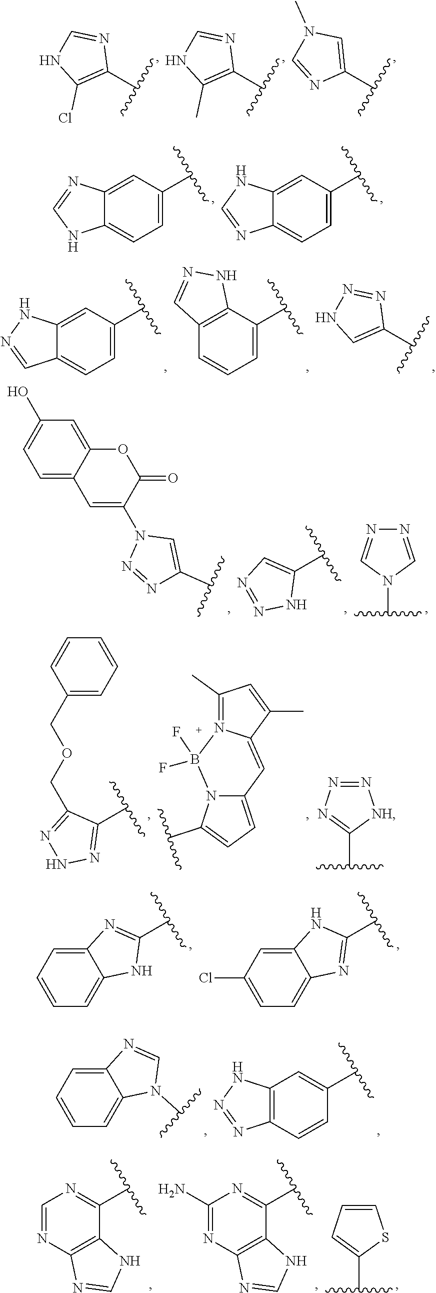

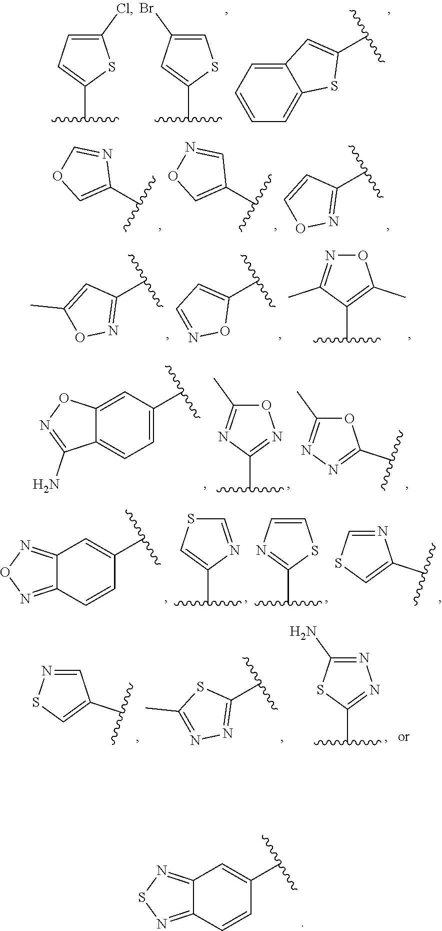

In some embodiments, R¹ is

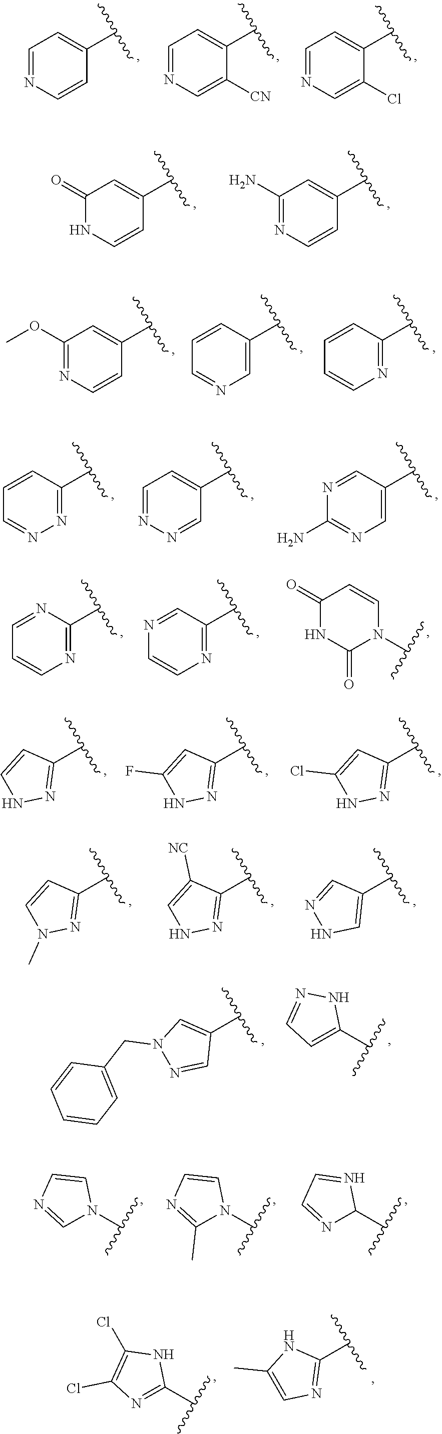

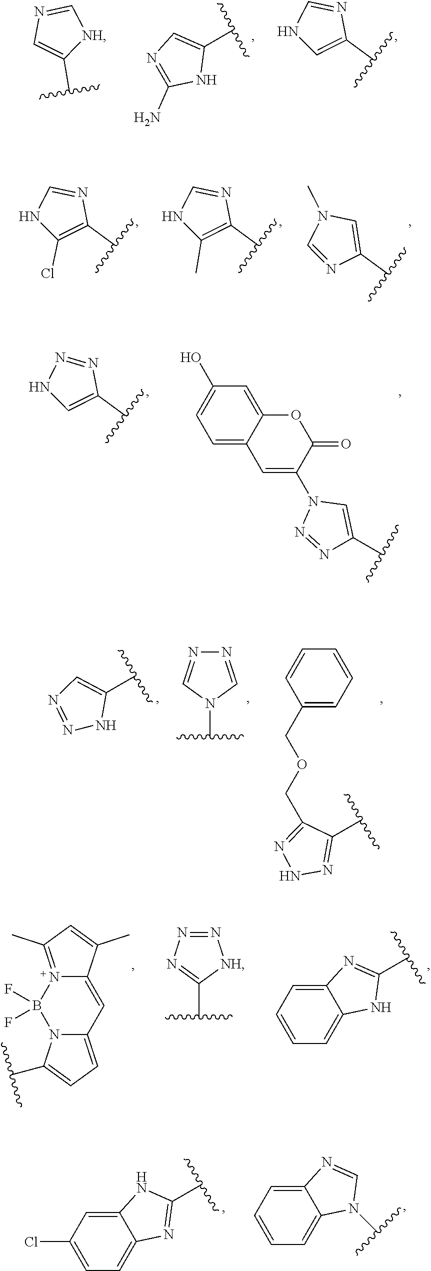

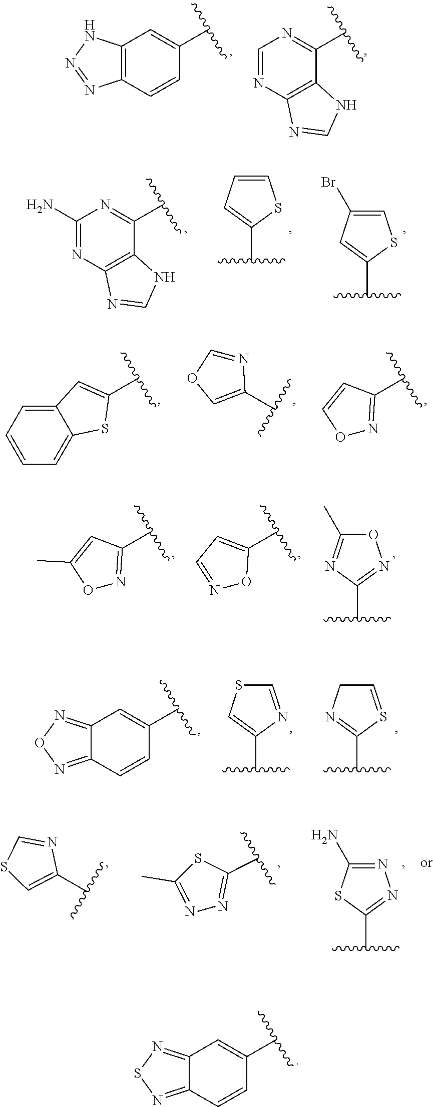

In some embodiments, R¹ is

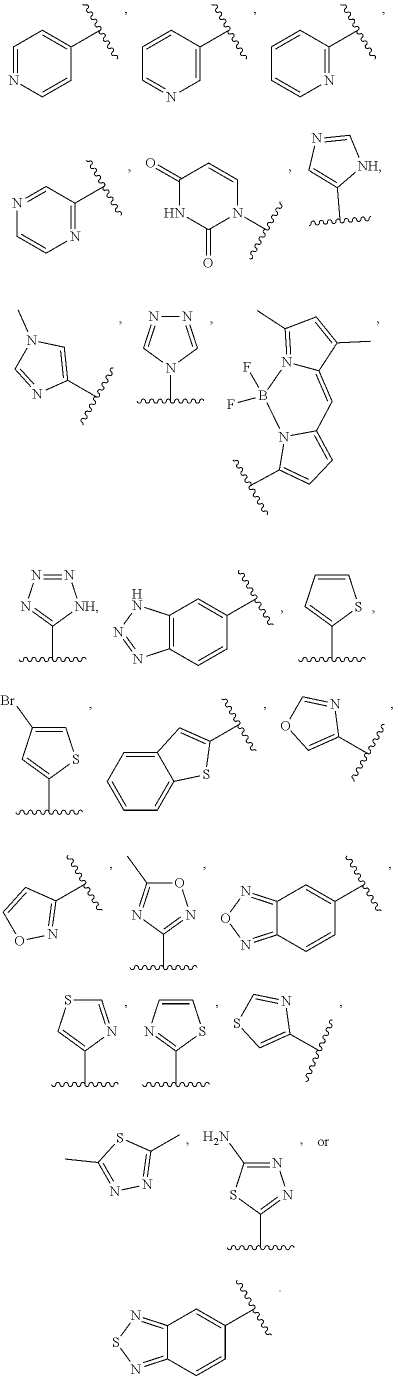

In some embodiments, R¹ is

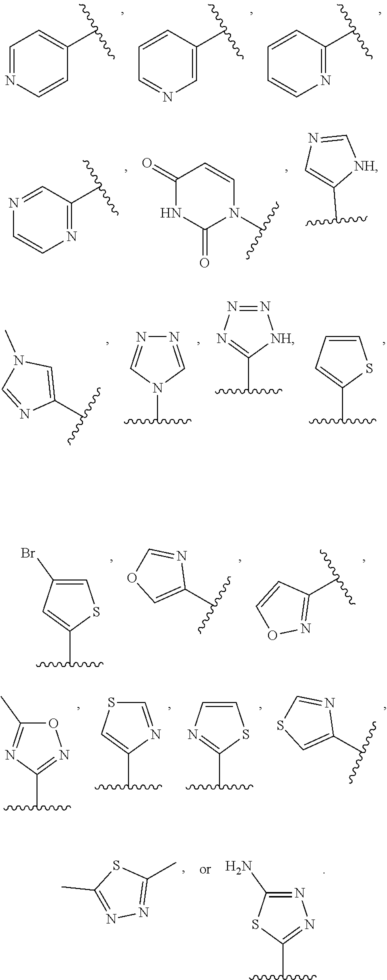

In some embodiments, R¹ is

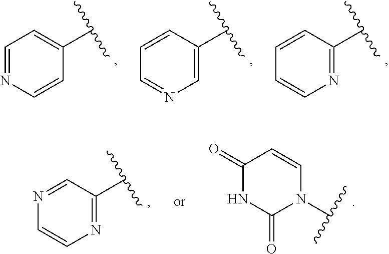 [SMILES: *c1ccncc1 ; *c1cccnc1 ; *c1ccccn1 ; *c1cnccn1 ; *n1ccc(=O)[nH]c1=O]

In some embodiments, R¹ is

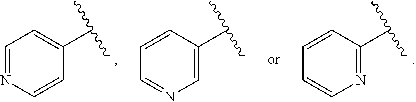 [SMILES: *c1ccncc1 ; *c1cccnc1 ; *c1ccccn1]

In some embodiments, R¹ is

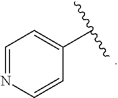 [SMILES: *c1ccncc1]

In some embodiments, R¹ is

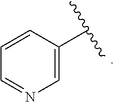 [SMILES: *c1cccnc1]

In some embodiments, R¹ is

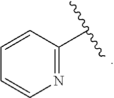 [SMILES: *c1ccccn1]

In some embodiments, R¹ is

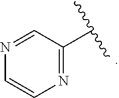 [SMILES: *c1cnccn1]

In some embodiments, R¹ is

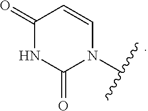 [SMILES: *n1ccc(=O)[nH]c1=O]

In some embodiments, R¹ is

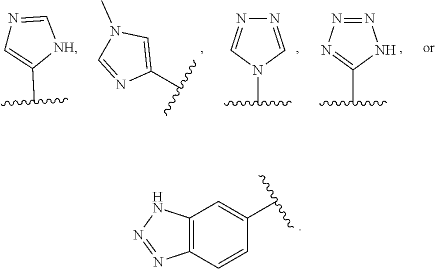

In some embodiments, R¹ is

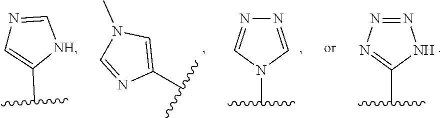

In some embodiments, R¹ is

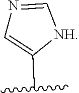 [SMILES: *c1cnc[nH]1]

In some embodiments, R¹ is

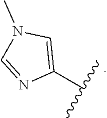 [SMILES: *c1cn(C)cn1]

In some embodiments, R¹ is

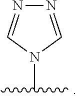 [SMILES: *n1cnnc1]

In some embodiments, R¹ is

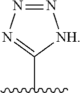 [SMILES: *c1nnn[nH]1]

In some embodiments, R¹ is

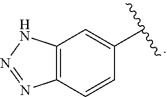 [SMILES: *c1ccc2nn[nH]c2c1]

In some embodiments, R¹ is

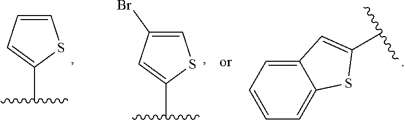 [SMILES: *c1cccs1 ; *c1cc(Br)cs1 ; *c1cc2ccccc2s1]

In some embodiments, R¹ is

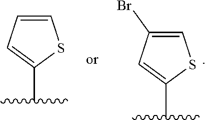 [SMILES: *c1cccs1 ; *c1cc(Br)cs1]

In some embodiments, R¹ is

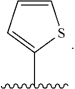 [SMILES: *c1cccs1]

In some embodiments, R¹ is

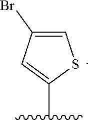 [SMILES: *c1cc(Br)cs1]

In some embodiments, R¹ is

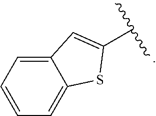 [SMILES: *c1cc2ccccc2s1]

In some embodiments, R¹ is

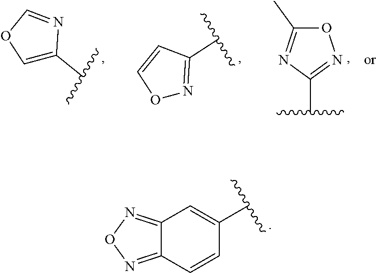

In some embodiments, R¹ is

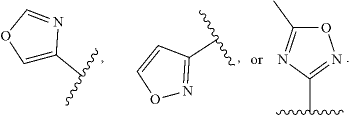

In some embodiments, R¹ is

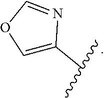 [SMILES: *c1cocn1]

In some embodiments, R¹ is

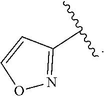 [SMILES: *c1ccon1]

In some embodiments, R¹ is

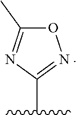 [SMILES: *c1noc(C)n1]

In some embodiments, R¹ is

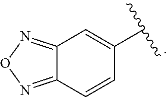 [SMILES: *c1ccc2nonc2c1]

In some embodiments, R¹ is

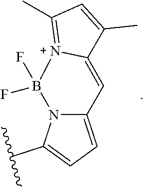

In some embodiments, R¹ is

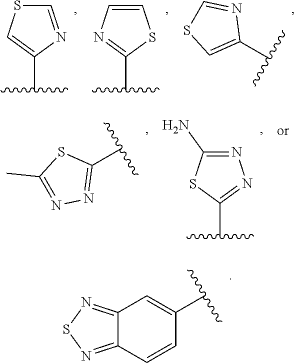

In some embodiments, R¹ is

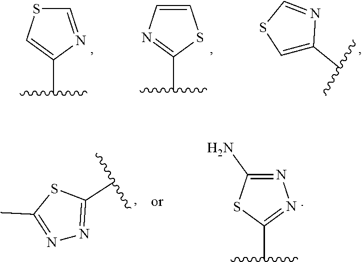

In some embodiments, R¹ is

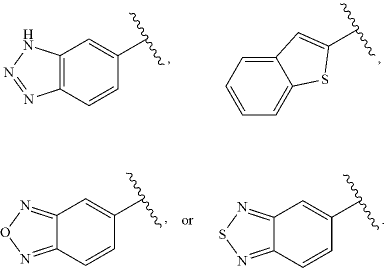 [SMILES: *c1ccc2nn[nH]c2c1 ; *c1cc2ccccc2s1 ; *c1ccc2nonc2c1 ; *c1ccc2nsnc2c1]

In some embodiments, R¹ is

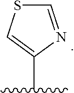 [SMILES: *c1cscn1]

In some embodiments, R¹ is

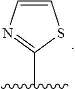 [SMILES: *c1nccs1]

In some embodiments, R¹ is

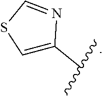 [SMILES: *c1cscn1]

In some embodiments, R¹ is

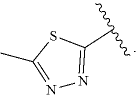 [SMILES: *c1nnc(C)s1]

In some embodiments, R¹ is

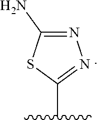 [SMILES: *c1nnc(N)s1]

In some embodiments, R¹ is

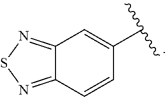 [SMILES: *c1ccc2nsnc2c1]

In some embodiments, R¹ is —OR^{1a} or —N(R^{1a})₂. In some embodiments, R¹ is —OR^{1a}. In some embodiments, R¹ is —OH. In some embodiments, R¹ is —O(optionally substituted alkyl). In some embodiments, R¹ is —O(optionally substituted C₁₋₆ alkyl). In some embodiments, R¹ is —O(optionally substituted methyl), —O(optionally substituted ethyl), —O(optionally substituted n-propyl), —O(optionally substituted isopropyl), —O(optionally substituted n-butyl), —O(optionally substituted tert-butyl), —O(optionally substituted sec-butyl), —O(optionally substituted isobutyl), —O(optionally substituted n-pentyl), —O(optionally substituted 3-pentanyl), —O(optionally substituted amyl), —O(optionally substituted neopentyl), —O(optionally substituted 3-methyl-2-butanyl), —O(optionally substituted tert-amyl), or —O(optionally substituted n-hexyl). In some embodiments, R¹ is —O(optionally substituted ethyl). In some embodiments, R¹ is —O(optionally substituted isopropyl). In some embodiments, R¹ is —O(optionally substituted isobutyl).

In some embodiments, R¹ is —O(alkyl substituted with —OR^{1a}). In some embodiments, R¹ is —O(C₁₋₆ alkyl substituted with —OR^{1a}). In some embodiments, R¹ is —O(ethyl substituted with —OR^{1a}). In some embodiments, R¹ is —O(alkyl substituted with —O(optionally substituted alkyl)). In some embodiments, R¹ is —O(C₁₋₆ alkyl substituted with —O(optionally substituted alkyl)). In some embodiments, R¹ is —O(ethyl substituted with —O(optionally substituted alkyl)). In some embodiments, R¹ is —O(alkyl substituted with —O(unsubstituted alkyl)). In some embodiments, R¹ is —O(C₁₋₆ alkyl substituted with —O(unsubstituted alkyl)). In some embodiments, R¹ is —O(ethyl substituted with —O(unsubstituted alkyl)). In some embodiments, R¹ is —O(alkyl substituted with —OCH₃). In some embodiments, R¹ is —O(C₁₋₆ alkyl substituted with —OCH₃)). In some embodiments, R¹ is —O(ethyl substituted with —OCH₃)).

In some embodiments, R¹ is —N(R^{1a})₂. In some embodiments, R¹ is —NR^{1a}(optionally substituted alkyl). In some embodiments, R¹ is —NR^{1a}(optionally substituted C₁₋₆ alkyl). In some embodiments, R¹ is —NR^{1a}(optionally substituted methyl), —NR^{1a}(optionally substituted ethyl), —NR^{1a}(optionally substituted n-propyl), —NR^{1a}(optionally substituted isopropyl), —NR^{1a}(optionally substituted n-butyl), —NR^{1a}(optionally substituted tert-butyl), —NR^{1a}(optionally substituted sec-butyl), —NR^{1a}(optionally substituted isobutyl), —NR^{1a}(optionally substituted n-pentyl), —NR^{1a}(optionally substituted 3-pentanyl), —NR^{1a}(optionally substituted amyl), —NR^{1a}(optionally substituted neopentyl), —NR^{1a}(optionally substituted 3-methyl-2-butanyl), —NR^{1a}(optionally substituted tert-amyl), or —NR^{1a}(optionally substituted n-hexyl). In some embodiments, R¹ is —NR^{1a}(optionally substituted methyl). In some embodiments, R¹ is —NR^{1a}(optionally substituted ethyl). In some embodiments, R¹ is —NR^{1a}(optionally substituted isopropyl). In some embodiments, R¹ is —NR^{1a}(optionally substituted n-butyl). In some embodiments, R¹ is —NR^{1a}(substituted methyl). In some embodiments, R¹ is —NR^{1a}(substituted ethyl). In some embodiments, R¹ is —NR^{1a}(substituted isopropyl). In some embodiments, R¹ is —NR^{1a}(substituted n-butyl). In some embodiments, R¹ is —NR^{1a}(unsubstituted methyl). In some embodiments, R¹ is —NR^{1a}(unsubstituted ethyl). In some embodiments, R¹ is —NR^{1a}(unsubstituted isopropyl). In some embodiments, R¹ is —NR^{1a}(unsubstituted n-butyl). In some embodiments, R¹ is —N(optionally substituted alkyl)₂. In some embodiments, R¹ is —N(optionally substituted C₁₋₆ alkyl)₂.

In some embodiments, R¹ is —NR^{1a}(alkyl substituted with ═O), —NR^{1a}(alkyl substituted with —OR^{1a}), —NR^{1a}(alkyl substituted with optionally substituted carbocyclyl), —NR^{1a}(alkyl substituted with optionally substituted aryl), or —NR^{1a}(alkyl substituted with optionally substituted heteroaryl). In some embodiments, R¹ is —NR^{1a}(alkyl substituted with ═O). In some embodiments, R¹ is —NR^{1a}(C₁₋₆ alkyl substituted with ═O). In some embodiments, R¹ is —NR^{1a}(methyl substituted with ═O).

In some embodiments, R¹ is —NR^{1a}(alkyl substituted with —OR^{1a}). In some embodiments, R¹ is —NR^{1a}(C₁₋₆ alkyl substituted with —OR^{1a}). In some embodiments, R¹ is —NR^{1a}(ethyl substituted with —OR^{1a}). In some embodiments, R¹ is —NR^{1a}(alkyl substituted with —O(optionally substituted C₁₋₆ alkyl)). In some embodiments, R¹ is —NR^{1a}(C₁₋₆ alkyl substituted with —O(optionally substituted C₁₋₆ alkyl)). In some embodiments, R¹ is —NR^{1a}(ethyl substituted with —O(optionally substituted C₁₋₆ alkyl)). In some embodiments, R¹ is —NR^{1a}(C₁₋₆ alkyl substituted with —O(unsubstituted C₁₋₆ alkyl)). In some embodiments, R¹ is —NR^{1a}(alkyl substituted with —OCH₃). In some embodiments, R¹ is —NR^{1a}(C₁₋₆ alkyl substituted with —OCH₃). In some embodiments, R¹ is —NR^{1a}(ethyl substituted with —OCH₃).

In some embodiments, R¹ is —NR^{1a}(alkyl substituted with optionally substituted carbocyclyl). In some embodiments, R¹ is —NR^{1a}(alkyl substituted with optionally substituted C₃₋₆ carbocyclyl). In some embodiments, R¹ is —NR^{1a}(alkyl substituted with substituted C₃₋₆ carbocyclyl). In some embodiments, R¹ is —NR^{1a}(alkyl substituted with unsubstituted C₃₋₆ carbocyclyl).

In some embodiments, R¹ is —NR^{1a}(alkyl substituted with optionally substituted aryl). In some embodiments, R¹ is —NR^{1a}(alkyl substituted with optionally substituted phenyl). In some embodiments, R¹ is —NR^{1a}(alkyl substituted with unsubstituted phenyl). In some embodiments, R¹ is —NR^{1a}(C₁₋₆ alkyl substituted with optionally substituted phenyl). In some embodiments, R¹ is —NR^{1a}(C₁₋₆ alkyl substituted with unsubstituted phenyl). In some embodiments, R¹ is —NR^{1a}(ethyl substituted with unsubstituted phenyl).

In some embodiments, R¹ is-NR^{1a}(alkyl substituted with optionally substituted heteroaryl). In some embodiments, R¹ is —NR^{1a}(alkyl substituted with optionally substituted imidazolyl) or —NR^{1a}(alkyl substituted with optionally substituted pyridyl). In some embodiments, R¹ is —NR^{1a}(C₁₋₆ alkyl substituted with optionally substituted imidazolyl) or —NR^{1a}(C₁₋₆ alkyl substituted with optionally substituted pyridyl). In some embodiments, R¹ is —NR^{1a}(C₁₋₆ alkyl substituted with unsubstituted imidazolyl) or —NR^{1a}(C₁₋₆ alkyl substituted with unsubstituted pyridyl). In some embodiments, R¹ is —NR^{1a}(ethyl substituted with unsubstituted imidazolyl) or —NR^{1a}(ethyl substituted with unsubstituted pyridyl).

In some embodiments, R¹ is —NHR^{1a}. In some embodiments, R¹ is —NH(optionally substituted alkyl). In some embodiments, R¹ is —NH(optionally substituted C₁₋₆ alkyl). In some embodiments, R¹ is —NH(optionally substituted methyl), —NH(optionally substituted ethyl), —NH(optionally substituted n-propyl), —NH(optionally substituted isopropyl), —NH(optionally substituted n-butyl), —N H(optionally substituted tert-butyl), —NH(optionally substituted sec-butyl), —NH(optionally substituted isobutyl), —NH(optionally substituted n-pentyl), —NH(optionally substituted 3-pentanyl), —N H(optionally substituted amyl), —NH(optionally substituted neopentyl), —NH(optionally substituted 3-methyl-2-butanyl), —NH(optionally substituted tert-amyl), or —NH(optionally substituted n-hexyl). In some embodiments, R¹ is —NH(optionally substituted isopropyl). In some embodiments, R¹ is —NH(substituted isopropyl). In some embodiments, R¹ is —NH(unsubstituted isopropyl). In some embodiments, R¹ is —NH₂. In some embodiments, R¹ is —NH(unsubstituted isopropyl) or —NH₂.

In some embodiments, R¹ is —OH, —OCHF₂, —OCF₃,

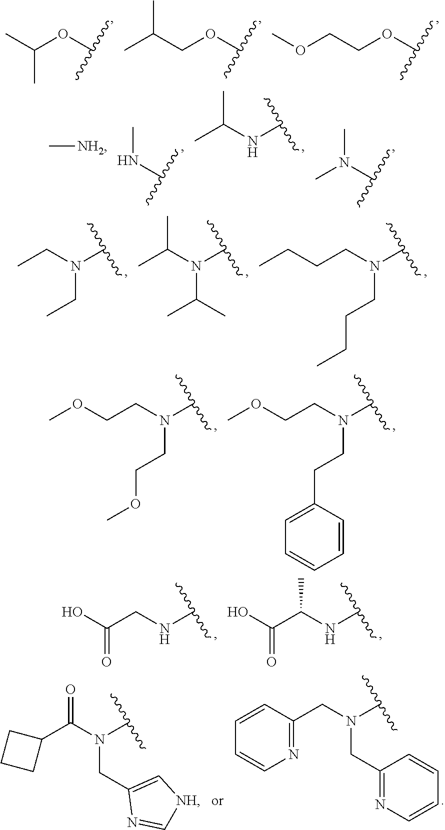

In some embodiments, R¹ is

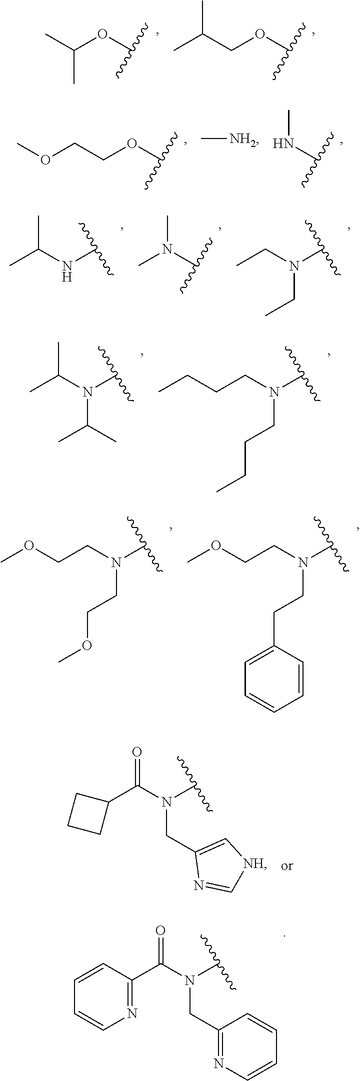

In some embodiments, R¹ is —OH,

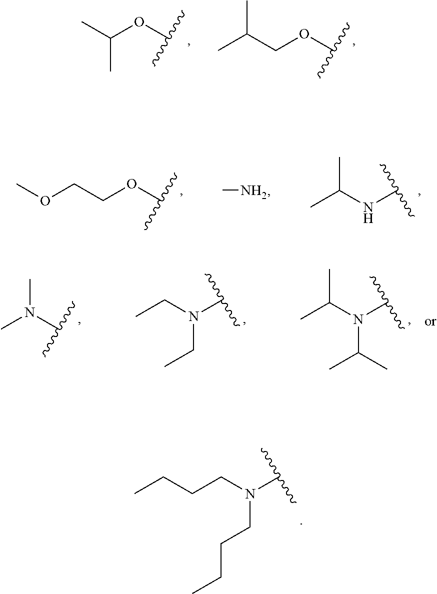

In some embodiments, R¹ is —OH,

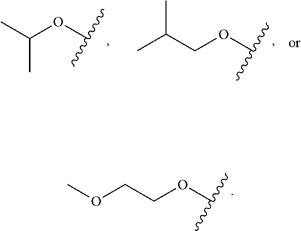

In some embodiments, R¹ is —OH or

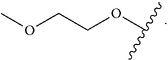 [SMILES: *OCCOC]

In some embodiments, R¹ is —NH₂,

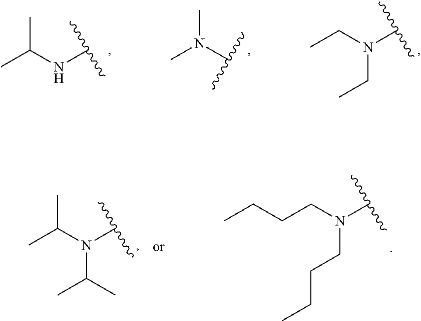

In some embodiments, R¹ is —NH₂ or

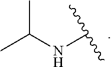 [SMILES: *NC(C)C]

In some embodiments, R¹ is —OH,

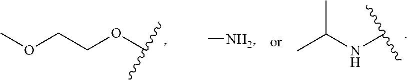

As generally described herein, R² is hydrogen or optionally substituted alkyl. In some embodiments, R² is hydrogen. In some embodiments, R² is optionally substituted alkyl. In some embodiments, R² is optionally substituted C₁₋₁₂ alkyl. In some embodiments, R² is optionally substituted C₁₋₆ alkyl. In some embodiments, R² is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R² is substituted C₁₋₆ alkyl. In some embodiments, R² is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R² is unsubstituted C₁₋₆ alkyl. In some embodiments, R² is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R² is optionally substituted methyl. In some embodiments, R² is substituted methyl. In some embodiments, R² is methyl.

As generally described herein, n is 0, 1, 2, 3, 4, or 5. In some embodiments, n is 0 or 1. In some embodiments, n is 0. In some embodiments, n is 1. In some embodiments, n is 2. In some embodiments, n is 3. In some embodiments, n is 4. In some embodiments, n is 5.

As generally described herein, each instance of R^{Y} is independently hydrogen or halogen, or two instances of R^{Y} are taken together to form ═O. In some embodiments, each instance of R is independently hydrogen or halogen. In some embodiments, at least one instance of R^{Y} is hydrogen. In some embodiments, at least one instance of R^{Y} is halogen. In some embodiments, at least one instance of R^{Y} is —F. In some embodiments, each instance of R is hydrogen. In some embodiments, each instance of R^{Y} is halogen. In some embodiments, each instance of R^{Y} is —F. In some embodiments, two instances of R^{Y} are taken together to form ═O.

In some embodiments, each instance of Y is independently —C(R^{Y})₂—, —O—, or —N(R^{1a})—. In some embodiments, at least one instance of Y is —C(R^{Y})₂—. In some embodiments, at least one instance of Y is —CHR^{Y}—. In some embodiments, at least one instance of Y is —CH(CH₃)—. In some embodiments, at least one instance of Y is —CH₂—. In some embodiments, at least one instance of Y is —CFR^{Y}—. In some embodiments, at least one instance of Y is —CHF—. In some embodiments, at least one instance of Y is —CF₂—. In some embodiments, at least one instance of Y is —C(═O)—. In some embodiments, at least one instance of Y is —O— or —N(R^{1a})—. In some embodiments, at least one instance of Y is —O—. In some embodiments, at least one instance of Y is —N(R^{1a})—. In some embodiments, at least one instance of Y is —NH—. In some embodiments, at least one instance of Y is —N(R^{1a})—, and R^{1a} is optionally substituted alkyl or nitrogen protecting group. In some embodiments, at least one instance of Y is —N(R^{1a})—, and R^{1a} is optionally substituted C₁₋₆ alkyl. In some embodiments, at least one instance of Y is —N(R^{1a})—, and R^{1a} is nitrogen protecting group.

In some embodiments, (Y)ₙ is —CH₂—. In some embodiments, X is —CH₂—, and (Y)ₙ is —CH₂—.

In some embodiments, (Y)ₙ is —C(═O)—. In some embodiments, X is —CH₂—, and (Y)ₙ is —C(═O)—. In some embodiments, (Y)ₙ is —CH₂C(═O)— or —C(═O)CH₂—. In some embodiments, X is —CH₂—, and (Y)ₙ is —CH₂C(═O)— or —C(═O)CH₂—. In some embodiments, (Y)ₙ is —NH—. In some embodiments, X is —CH₂—, and (Y)ₙ is —NH—. In some embodiments, (Y)ₙ is —NHCH₂— or —CH₂NH—. In some embodiments, X is —CH₂—, and (Y)ₙ is —NHCH₂— or —CH₂NH—. In some embodiments, (Y)ₙ is —C(═O)NHCH₂— or —CH₂NHC(═O)—. In some embodiments, X is —CH₂—, and (Y)ₙ is —C(═O)NHCH₂— or —CH₂NHC(═O)—.

In some embodiments, R¹ and R² are joined together with their intervening atoms to form optionally substituted carbocyclyl or optionally substituted heterocyclyl. In some embodiments, R¹ and R² are joined together with their intervening atoms to form optionally substituted carbocyclyl. In some embodiments, R¹ and R² are joined together with their intervening atoms to form optionally substituted C₃₋₁₀ carbocyclyl. In some embodiments, R¹ and R² are joined together with their intervening atoms to form optionally substituted C₃₋₆ carbocyclyl. In some embodiments, R¹ and R² are joined together with their intervening atoms to form optionally substituted cyclopropyl, optionally substituted cyclobutyl, optionally substituted cyclopentyl, or optionally substituted cyclohexyl. In some embodiments, R¹ and R² are joined together with their intervening atoms to form substituted cyclopropyl, substituted cyclobutyl, substituted cyclopentyl, or substituted cyclohexyl. In some embodiments, R¹ and R² are joined together with their intervening atoms to form unsubstituted cyclopropyl, unsubstituted cyclobutyl, unsubstituted cyclopentyl, or unsubstituted cyclohexyl.

In some embodiments, R¹ and R² are joined together with their intervening atoms to form optionally substituted cyclobutyl, optionally substituted cyclohexyl, optionally substituted spiro[2.2]pentyl, optionally substituted spiro[2.3]hexyl, or optionally substituted spiro[3.3]heptyl. In some embodiments, R¹ and R² are joined together with their intervening atoms to form optionally substituted cyclobutyl. In some embodiments, R¹ and R² are joined together with their intervening atoms to form optionally substituted cyclohexyl. In some embodiments, R¹ and R² are joined together with their intervening atoms to form optionally substituted spiro[2.2]pentyl. In some embodiments, R¹ and R² are joined together with their intervening atoms to form optionally substituted spiro[2.3]hexyl. In some embodiments, R¹ and R² are joined together with their intervening atoms to form optionally substituted spiro[3.3]heptyl. In some embodiments, R¹ and R² are joined together with their intervening atoms to form substituted cyclobutyl, substituted cyclohexyl, substituted spiro[2.2]pentyl, substituted spiro[2.3]hexyl, or substituted spiro[3.3]heptyl. In some embodiments, R¹ and R² are joined together with their intervening atoms to form substituted cyclobutyl. In some embodiments, R¹ and R² are joined together with their intervening atoms to form substituted cyclohexyl. In some embodiments, R¹ and R² are joined together with their intervening atoms to form substituted spiro[2.2]pentyl. In some embodiments, R¹ and R² are joined together with their intervening atoms to form substituted spiro[2.3]hexyl. In some embodiments, R¹ and R² are joined together with their intervening atoms to form substituted spiro[3.3]heptyl. In some embodiments, R¹ and R² are joined together with their intervening atoms to form unsubstituted cyclobutyl, unsubstituted cyclohexyl, unsubstituted spiro[2.2]pentyl, unsubstituted spiro[2.3]hexyl, or unsubstituted spiro[3.3]heptyl. In some embodiments, R¹ and R² are joined together with their intervening atoms to form unsubstituted cyclobutyl. In some embodiments, R¹ and R² are joined together with their intervening atoms to form unsubstituted cyclohexyl. In some embodiments, R¹ and R² are joined together with their intervening atoms to form unsubstituted spiro[2.2]pentyl. In some embodiments, R¹ and R² are joined together with their intervening atoms to form unsubstituted spiro[2.3]hexyl. In some embodiments, R¹ and R² are joined together with their intervening atoms to form unsubstituted spiro[3.3]heptyl.

In some embodiments, R¹ and R² are joined together with their intervening atoms to form carbocyclyl substituted with halogen, optionally substituted alkyl, —OR^{1a}, and/or —N(R^{1a})₂. In some embodiments, R¹ and R² are joined together with their intervening atoms to form carbocyclyl substituted with halogen. In some embodiments, R¹ and R² are joined together with their intervening atoms to form carbocyclyl substituted with —F, —Cl, or —Br. In some embodiments, R¹ and R² are joined together with their intervening atoms to form carbocyclyl substituted with —F. In some embodiments, R¹ and R² are joined together with their intervening atoms to form cyclobutyl substituted with halogen, cyclohexyl substituted with halogen, spiro[2.2]pentyl substituted with halogen, spiro[2.3]hexyl substituted with halogen, or spiro[3.3]heptyl substituted with halogen. In some embodiments, R¹ and R² are joined together with their intervening atoms to form cyclobutyl substituted with —F. In some embodiments, R¹ and R² are joined together with their intervening atoms to form spiro[2.3]hexyl substituted with —F.

In some embodiments, R¹ and R² are joined together with their intervening atoms to form carbocyclyl substituted with optionally substituted alkyl. In some embodiments, R¹ and R² are joined together with their intervening atoms to form carbocyclyl substituted with optionally substituted C₁₋₆ alkyl. In some embodiments, R¹ and R² are joined together with their intervening atoms to form carbocyclyl substituted with C₁₋₆ alkyl substituted with halogen. In some embodiments, R¹ and R² are joined together with their intervening atoms to form carbocyclyl substituted with C₁₋₆ alkyl substituted with —F. In some embodiments, R¹ and R² are joined together with their intervening atoms to form carbocyclyl substituted with —CH₂F, —CHF₂, or —CF₃. In some embodiments, R¹ and R² are joined together with their intervening atoms to form carbocyclyl substituted with —CF₃. In some embodiments, R¹ and R² are joined together with their intervening atoms to form cyclobutyl substituted with optionally substituted alkyl, cyclohexyl substituted with optionally substituted alkyl, spiro[2.2]pentyl substituted with optionally substituted alkyl, spiro[2.3]hexyl substituted with optionally substituted alkyl, or spiro[3.3]heptyl substituted with optionally substituted alkyl. In some embodiments, R¹ and R² are joined together with their intervening atoms to form cyclobutyl substituted with —CF₃.

In some embodiments, R¹ and R² are joined together with their intervening atoms to form carbocyclyl substituted with —OR^{1a}. In some embodiments, R¹ and R² are joined together with their intervening atoms to form cyclobutyl substituted with —OR^{1a}, cyclohexyl substituted with —OR^{1a}, spiro[2.2]pentyl substituted with —OR^{1a}, spiro[2.3]hexyl substituted with —OR^{1a}, or spiro[3.3]heptyl substituted with —OR^{1a}. In some embodiments, R¹ and R² are joined together with their intervening atoms to form carbocyclyl substituted with —OH. In some embodiments, R¹ and R² are joined together with their intervening atoms to form cyclobutyl substituted with —OH, cyclohexyl substituted with —OH, spiro[2.2]pentyl substituted with —OH, spiro[2.3]hexyl substituted with —OH, or spiro[3.3]heptyl substituted with —OH.

In some embodiments, R¹ and R² are joined together with their intervening atoms to form carbocyclyl substituted with —N(R^{1a})₂. In some embodiments, R¹ and R² are joined together with their intervening atoms to form cyclobutyl substituted with —N(R^{1a})₂, cyclohexyl substituted with —N(R^{1a})₂, spiro[2.2]pentyl substituted with —N(R^{1a})₂, spiro[2.3]hexyl substituted with —N(R^{1a})₂, or spiro[3.3]heptyl substituted with —N(R^{1a})₂. In some embodiments, R¹ and R² are joined together with their intervening atoms to form carbocyclyl substituted with —NR^{1a}(optionally substituted alkyl). In some embodiments, R¹ and R² are joined together with their intervening atoms to form carbocyclyl substituted with —NR^{1a}(optionally substituted C₁₋₆ alkyl). In some embodiments, R¹ and R² are joined together with their intervening atoms to form carbocyclyl substituted with —NR^{1a}(optionally substituted methyl). In some embodiments, R¹ and R² are joined together with their intervening atoms to form carbocyclyl substituted with —NR^{1a}CH₃. In some embodiments, R¹ and R² are joined together with their intervening atoms to form carbocyclyl substituted with —N(CH₃)₂. In some embodiments, R¹ and R² are joined together with their intervening atoms to form cyclobutyl substituted with —N(CH₃)₂, cyclohexyl substituted with —N(CH₃)₂, spiro[2.2]pentyl substituted with —N(CH₃)₂, spiro[2.3]hexyl substituted with —N(CH₃)₂, or spiro[3.3]heptyl substituted with —N(CH₃)₂.

In some embodiments, R¹ and R² are joined together with their intervening atoms to form

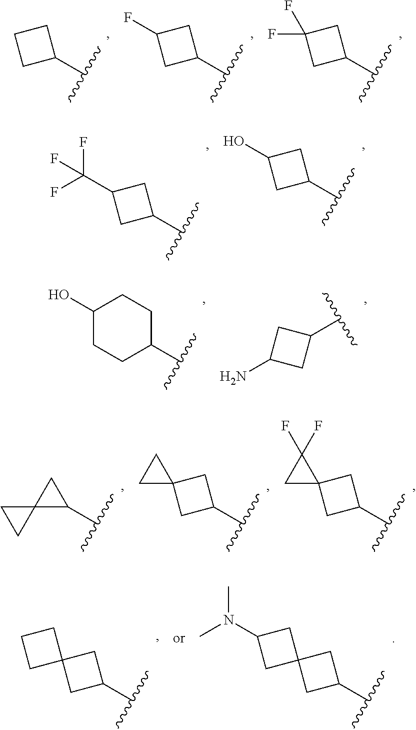

In some embodiments, R¹ and R² are joined together with their intervening atoms to form

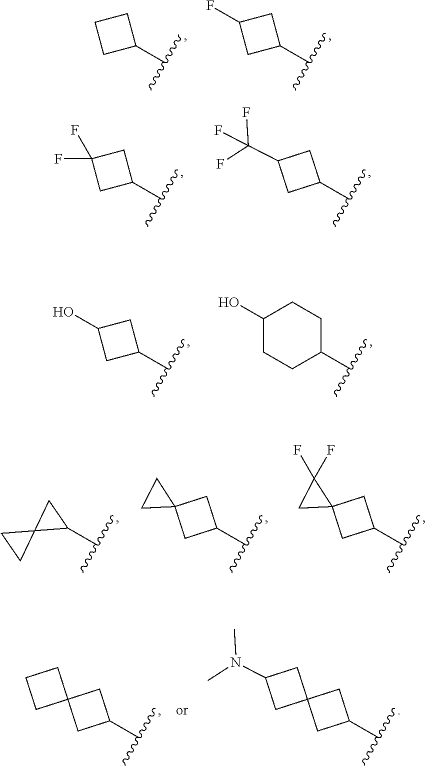

In some embodiments, R¹ and R² are joined together with their intervening atoms to form

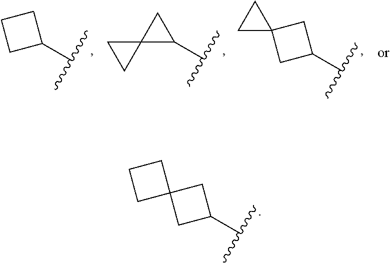

In some embodiments, R¹ and R² are joined together with their intervening atoms to form

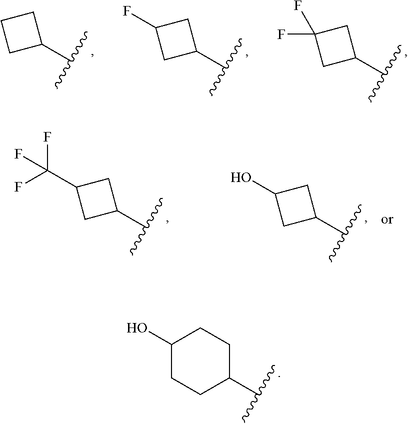 [SMILES: *C1CCC1 ; *C1CC(F)C1 ; *C1CC(F)(F)C1 ; *C1CC(C(F)(F)F)C1 ; *C1CC(O)C1 ; *C1CCC(O)CC1]

In some embodiments, R¹ and R² are joined together with their intervening atoms to form

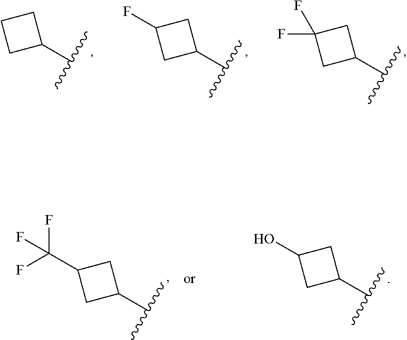 [SMILES: *C1CCC1 ; *C1CC(F)C1 ; *C1CC(F)(F)C1 ; *C1CC(C(F)(F)F)C1 ; *C1CC(O)C1]

In some embodiments, R¹ and R² are joined together with their intervening atoms to form

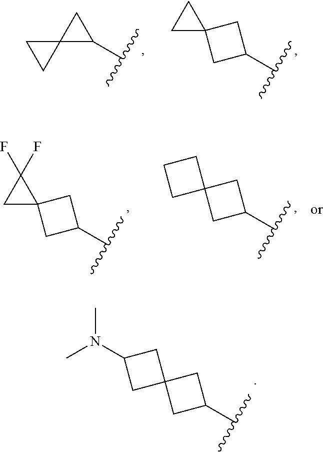 [SMILES: *C1CC12CC2 ; *C1CC2(CC2)C1 ; *C1CC2(C1)CC2(F)F ; *C1CC2(CCC2)C1 ; *C1CC2(C1)CC(N(C)C)C2]

In some embodiments, R¹ and R² are joined together with their intervening atoms to form optionally substituted heterocyclyl. In some embodiments, R¹ and R² are joined together with their intervening atoms to form substituted 3-14 membered heterocyclyl. In some embodiments, R¹ and R² are joined together with their intervening atoms to form optionally substituted oxetanyl, optionally substituted tetrahydrofuranyl, optionally substituted tetrahydropyranyl, optionally substituted azetidinyl, optionally substituted pyrrolidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl. In some embodiments, R¹ and R² are joined together with their intervening atoms to form optionally substituted oxetanyl. In some embodiments, R¹ and R² are joined together with their intervening atoms to form optionally substituted tetrahydrofuranyl. In some embodiments, R¹ and R² are joined together with their intervening atoms to form optionally substituted tetrahydropyranyl. In some embodiments, R¹ and R² are joined together with their intervening atoms to form optionally substituted azetidinyl. In some embodiments, R¹ and R² are joined together with their intervening atoms to form optionally substituted pyrrolidinyl. In some embodiments, R¹ and R² are joined together with their intervening atoms to form optionally substituted 2-azaspiro[3.3]heptyl. In some embodiments, R¹ and R² are joined together with their intervening atoms to form optionally substituted 2,6-diazaspiro[3.4]octanyl. In some embodiments, R¹ and R² are joined together with their intervening atoms to form optionally substituted tetrahydrothiophenyl. In some embodiments, R¹ and R2 are joined together with their intervening atoms to form optionally substituted dithiolanyl. In some embodiments, R¹ and R² are joined together with their intervening atoms to form optionally substituted 1-imino-1-oxo-hexahydro-1λ⁶-thiopyranyl. In some embodiments, R¹ and R² are joined together with their intervening atoms to form substituted oxetanyl, substituted tetrahydrofuranyl, substituted tetrahydropyranyl, substituted azetidinyl, substituted pyrrolidinyl, substituted 2-azaspiro[3.3]heptyl, substituted 2,6-diazaspiro[3.4]octanyl, substituted tetrahydrothiophenyl, substituted dithiolanyl, or substituted 1-imino-1-oxo-hexahydro-1λ⁶-thiopyranyl. In some embodiments, R¹ and R² are joined together with their intervening atoms to form substituted oxetanyl.

In some embodiments, R¹ and R² are joined together with their intervening atoms to form substituted tetrahydrofuranyl. In some embodiments, R¹ and R² are joined together with their intervening atoms to form substituted tetrahydropyranyl. In some embodiments, R¹ and R² are joined together with their intervening atoms to form substituted azetidinyl. In some embodiments, R¹ and R² are joined together with their intervening atoms to form substituted pyrrolidinyl. In some embodiments, R¹ and R² are joined together with their intervening atoms to form substituted 2-azaspiro[3.3]heptyl. In some embodiments, R¹ and R² are joined together with their intervening atoms to form substituted 2,6-diazaspiro[3.4]octanyl. In some embodiments, R¹ and R² are joined together with their intervening atoms to form substituted tetrahydrothiophenyl. In some embodiments, R¹ and R² are joined together with their intervening atoms to form substituted dithiolanyl. In some embodiments, R¹ and R² are joined together with their intervening atoms to form substituted 1-imino-1-oxo-hexahydro-1λ⁶-thiopyranyl. In some embodiments, R¹ and R² are joined together with their intervening atoms to form unsubstituted oxetanyl, unsubstituted tetrahydrofuranyl, unsubstituted tetrahydropyranyl, unsubstituted azetidinyl, unsubstituted pyrrolidinyl, unsubstituted 2-azaspiro[3.3]heptyl, unsubstituted 2,6-diazaspiro[3.4]octanyl, unsubstituted tetrahydrothiophenyl, unsubstituted dithiolanyl, or unsubstituted 1-imino-1-oxo-hexahydro-1λ⁶-thiopyranyl. In some embodiments, R¹ and R² are joined together with their intervening atoms to form unsubstituted oxetanyl. In some embodiments, R¹ and R2 are joined together with their intervening atoms to form unsubstituted tetrahydrofuranyl. In some embodiments, R¹ and R² are joined together with their intervening atoms to form unsubstituted tetrahydropyranyl. In some embodiments, R¹ and R² are joined together with their intervening atoms to form unsubstituted azetidinyl. In some embodiments, R¹ and R² are joined together with their intervening atoms to form unsubstituted pyrrolidinyl. In some embodiments, R¹ and R² are joined together with their intervening atoms to form unsubstituted 2-azaspiro[3.3]heptyl. In some embodiments, R¹ and R² are joined together with their intervening atoms to form unsubstituted 2,6-diazaspiro[3.4]octanyl. In some embodiments, R¹ and R² are joined together with their intervening atoms to form unsubstituted tetrahydrothiophenyl. In some embodiments, R¹ and R² are joined together with their intervening atoms to form unsubstituted dithiolanyl. In some embodiments, R¹ and R2 are joined together with their intervening atoms to form unsubstituted 1-imino-1-oxo-hexahydro-1λ⁶-thiopyranyl.

In some embodiments, R¹ and R² are joined together with their intervening atoms to form optionally substituted oxetanyl, optionally substituted tetrahydrofuranyl, optionally substituted tetrahydropyranyl, optionally substituted azetidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl.

In some embodiments, R¹ and R² are joined together with their intervening atoms to form optionally substituted heterocyclyl substituted with optionally substituted alkyl or ═O. In some embodiments, R¹ and R² are joined together with their intervening atoms to form 3-14 membered heterocyclyl substituted with optionally substituted alkyl or ═O. In some embodiments, R¹ and R² are joined together with their intervening atoms to form 3-14 membered heterocyclyl substituted with optionally substituted alkyl. In some embodiments, R¹ and R² are joined together with their intervening atoms to form 3-14 membered heterocyclyl substituted with optionally substituted C₁₋₆ alkyl. In some embodiments, R¹ and R² are joined together with their intervening atoms to form 3-14 membered heterocyclyl substituted with methyl. In some embodiments, R¹ and R² are joined together with their intervening atoms to form 3-14 membered heterocyclyl substituted with ═O. In some embodiments, R¹ and R² are joined together with their intervening atoms to form 3-14 membered heterocyclyl substituted with optionally substituted alkyl and ═O.

In some embodiments, R¹ and R² are joined together with their intervening atoms to form

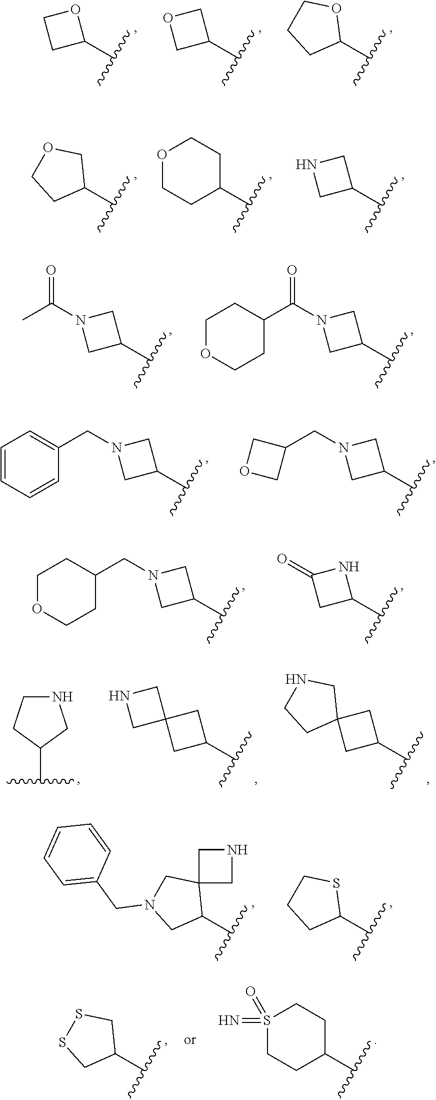

In some embodiments, R¹ and R² are joined together with their intervening atoms to form

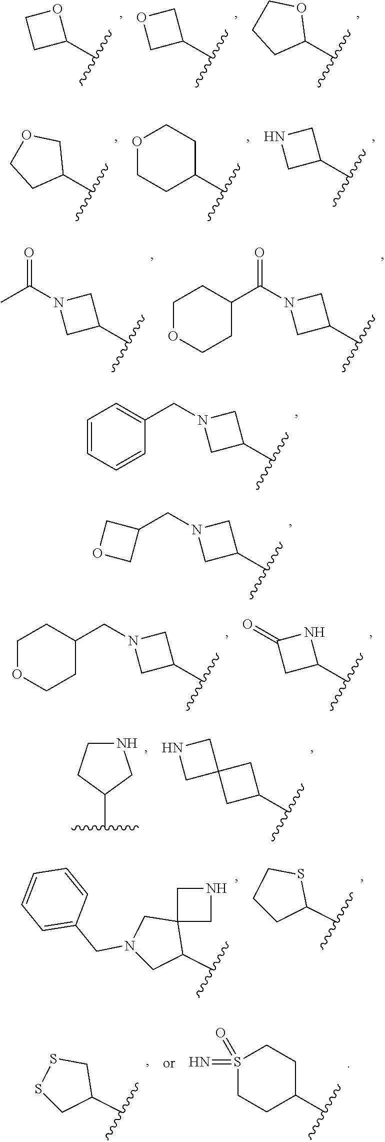

In some embodiments, R¹ and R² are joined together with their intervening atoms to form

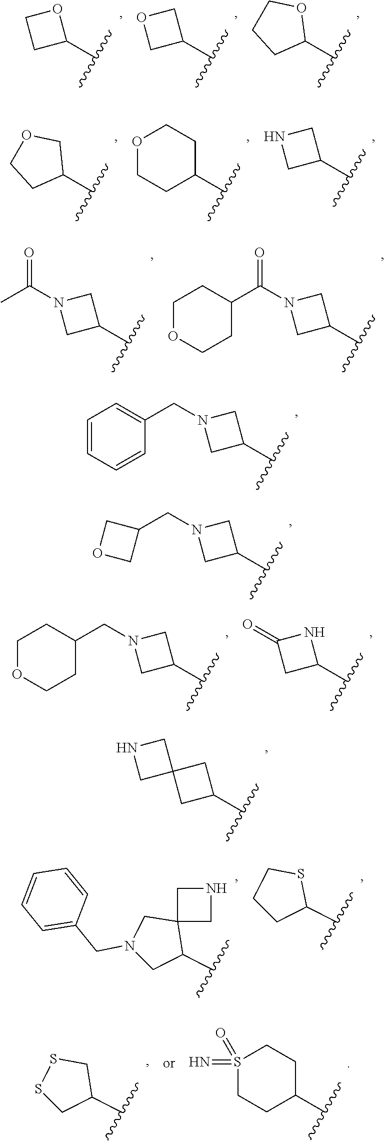

In some embodiments, R¹ and R² are joined together with their intervening atoms to form

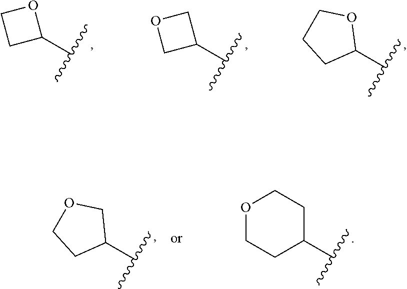 [SMILES: *C1CCO1 ; *C1COC1 ; *C1CCCO1 ; *C1CCOC1 ; *C1CCOCC1]

In some embodiments, R¹ and R² are joined together with their intervening atoms to form

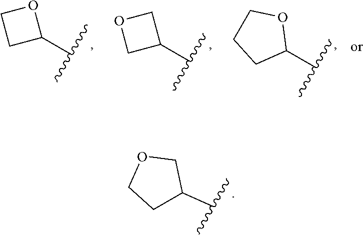 [SMILES: *C1CCO1 ; *C1COC1 ; *C1CCCO1 ; *C1CCOC1]

In some embodiments, R¹ and R^{Z} are joined together with their intervening atoms to form

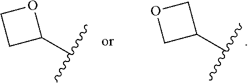 [SMILES: *C1CCO1 ; *C1COC1]

In some embodiments, R¹ and R² are joined together with their intervening atoms to form

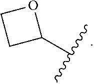 [SMILES: *C1CCO1]

In some embodiments, R¹ and R² are joined together with their intervening atoms to form

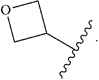 [SMILES: *C1COC1]

In some embodiments, R¹ and R² are joined together with their intervening atoms to form

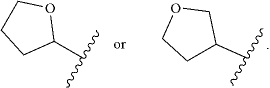 [SMILES: *C1CCCO1 ; *C1CCOC1]

In some embodiments, R¹ and R² are joined together with their intervening atoms to form

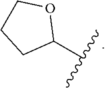 [SMILES: *C1CCCO1]

In some embodiments, R¹ and R² are joined together with their intervening atoms to form

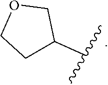 [SMILES: *C1CCOC1]

In some embodiments, R¹ and R² are joined together with their intervening atoms to form

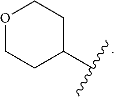 [SMILES: *C1CCOCC1]

In some embodiments, R¹ and R² are joined together with their intervening atoms to form

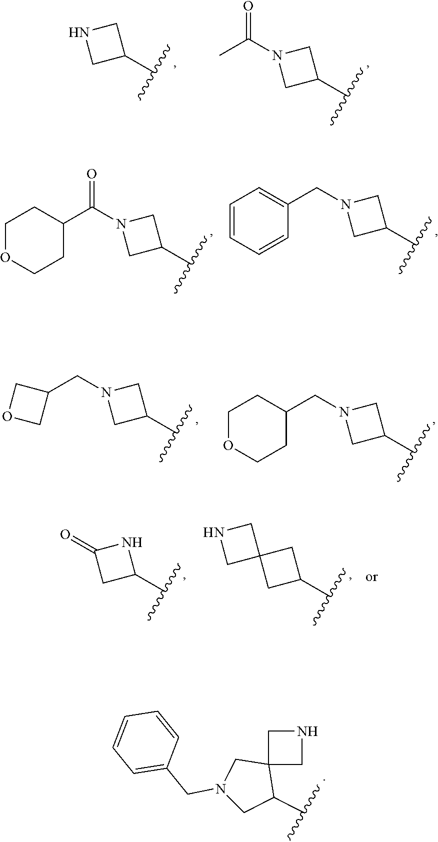

In some embodiments, R¹ and R² are joined together with their intervening atoms to form

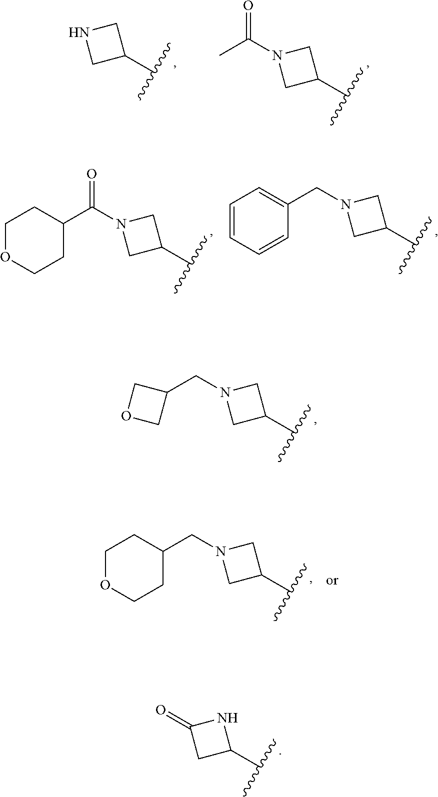

In some embodiments, R¹ and R² are joined together with their intervening atoms to form

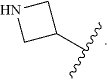 [SMILES: *C1CNC1]

In some embodiments, R¹ and R² are joined together with their intervening atoms to form

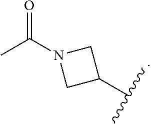 [SMILES: *C1CN(C(C)=O)C1]

In some embodiments, R¹ and R² are joined together with their intervening atoms to form

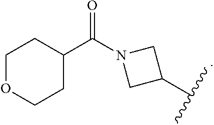 [SMILES: *C1CN(C(=O)C2CCOCC2)C1]

In some embodiments, R¹ and R² are joined together with their intervening atoms to form

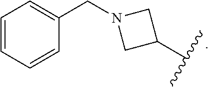 [SMILES: *C1CN(Cc2ccccc2)C1]

In some embodiments, R¹ and R² are joined together with their intervening atoms to form

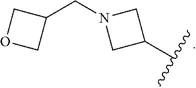 [SMILES: *C1CN(CC2COC2)C1]

In some embodiments, R¹ and R² are joined together with their intervening atoms to form

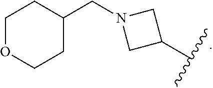 [SMILES: *C1CN(CC2CCOCC2)C1]

In some embodiments, R¹ and R² are joined together with their intervening atoms to form

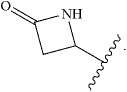 [SMILES: *C1CC(=O)N1]

In some embodiments, R¹ and R² are joined together with their intervening atoms to form

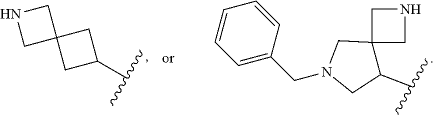 [SMILES: *C1CC2(CNC2)C1 ; *C1CN(Cc2ccccc2)CC12CNC2]

In some embodiments. R¹ and R² are joined together with their intervening atoms to form

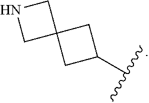 [SMILES: *C1CC2(CNC2)C1]

In some embodiments, R¹ and R² are joined together with their intervening atoms to form

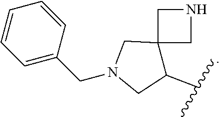 [SMILES: *C1CN(Cc2ccccc2)CC12CNC2]

In some embodiments, R¹ and R² are joined together with their intervening atoms to form

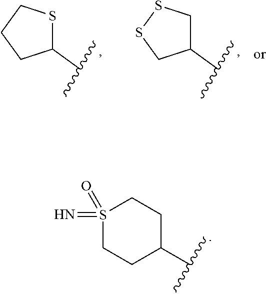 [SMILES: *C1CCCS1 ; *C1CSSC1 ; *C1CCS(=N)(=O)CC1]

In some embodiments, R¹ and R² are joined together with their intervening atoms to form

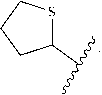 [SMILES: *C1CCCS1]

In some embodiments, R¹ and R² are joined together with their intervening atoms to form

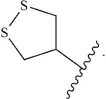 [SMILES: *C1CSSC1]

In some embodiments, R¹ and R² are joined together with their intervening atoms to form

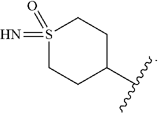 [SMILES: *C1CCS(=N)(=O)CC1]

As generally described herein, each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring.

In some embodiments, at least one instance of R^{1a} is hydrogen. In some embodiments, at least one instance of R^{1a} is optionally substituted acyl. In some embodiments, at least one instance of R^{1a} is optionally substituted alkyl. In some embodiments, at least one instance of Ria is optionally substituted C₁₋₁₂ alkyl. In some embodiments, at least one instance of Ria is optionally substituted C₁₋₆ alkyl. In some embodiments, at least one instance of R^{1a} is unsubstituted C₁₋₆ alkyl. In some embodiments, at least one instance of Ria is substituted C₁₋₆ alkyl. In some embodiments, at least one instance of R^{1a} is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, at least one instance of R^{1a} is hydrogen, optionally substituted methyl, optionally substituted ethyl, optionally substituted isopropyl, optionally substituted n-butyl, or optionally substituted isobutyl. In some embodiments, at least one instance of R^{1a} is hydrogen, methyl, ethyl, isopropyl, n-butyl, isobutyl

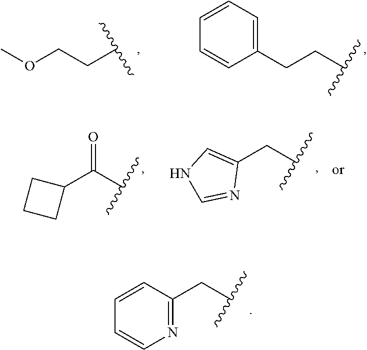

In some embodiments, at least one instance of R^{1a} is hydrogen, isopropyl, or

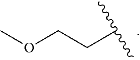 [SMILES: *CCOC]

In some embodiments, at least one instance of R^{1a} is optionally substituted C₂₋₁₂ alkenyl. In some embodiments, at least one instance of R^{1a} is optionally substituted C₂₋₆ alkenyl. In some embodiments, at least one instance of R^{1a} is optionally substituted C₂₋₁₂ alkynyl. In some embodiments, at least one instance of R^{1a} is optionally substituted C₂₋₆ alkynyl. In some embodiments, at least one instance of R^{1a} is optionally substituted heteroC₁₋₁₂ alkyl. In some embodiments, at least one instance of R^{1a} is optionally substituted heteroC₁₋₆ alkyl. In some embodiments, at least one instance of R^{1a} is optionally substituted heteroC₁₋₁₂ alkenyl. In some embodiments, at least one instance of R^{1a} is optionally substituted heteroC₁₋₆ alkenyl. In some embodiments, at least one instance of R^{1a} is optionally substituted heteroC₁₋₁₂ alkynyl. In some embodiments, at least one instance of R^{1a} is optionally substituted heteroC₁₋₄ alkynyl. In some embodiments, at least one instance of R^{1a} is optionally substituted C₃₋₁₄ cycloalkyl. In some embodiments, at least one instance of R^{1a} is optionally substituted 5-10 membered heterocyclyl. In some embodiments, at least one instance of R^{1a} is optionally substituted 6-14 membered aryl. In some embodiments, at least one instance of R^{1a} is optionally substituted 5-14 membered heteroaryl. In some embodiments, at least one instance of R^{1a} is a nitrogen protecting group when attached to a nitrogen atom. In some embodiments, at least one instance of R^{1a} is an oxygen protecting group when attached to an oxygen atom. In some embodiments, at least two instances of R^{1a} are joined together with their intervening atom to form an optionally substituted 5-10 membered heterocyclic ring. In some embodiments, at least two instances of R^{1a} are joined together with their intervening atom to form an optionally substituted 5-14 membered heteroaryl ring.

### R³

As generally described herein, R³ is optionally substituted carbocyclyl, optionally substituted heterocyclyl, optionally substituted aryl, or optionally substituted heteroaryl. In some embodiments, R³ is optionally substituted carbocyclyl. In some embodiments, R³ is optionally substituted C₃₋₁₀ carbocyclyl. In some embodiments, R³ is optionally substituted C₃₋₆ carbocyclyl. In some embodiments, R³ is optionally substituted heterocyclyl. In some embodiments, R³ is optionally substituted 3-14 membered heterocyclyl. In some embodiments, R³ is optionally substituted heteroaryl. In some embodiments, R³ is optionally substituted 4-10 membered heteroaryl. In some embodiments, R³ is

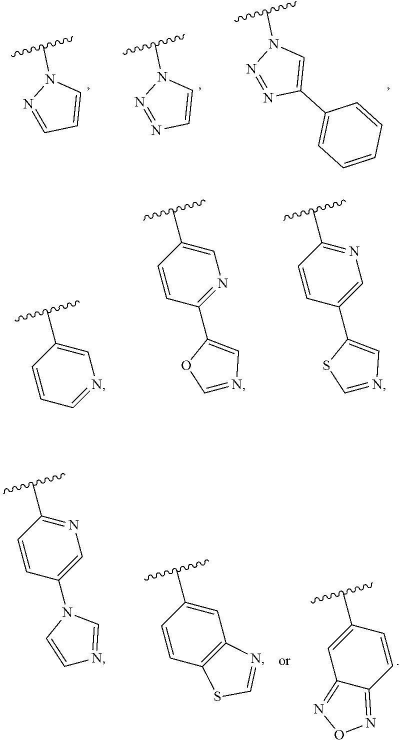

In some embodiments, R³ is optionally substituted aryl. In some embodiments, R³ is optionally substituted C₆₋₁₄ aryl. In some embodiments, R³ is optionally substituted phenyl. In some embodiments, R³ is unsubstituted phenyl. In some embodiments, R³ is substituted phenyl. In some embodiments, R³ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂. In some embodiments, R³ is phenyl substituted with halogen, optionally substituted alkyl, optionally substituted alkenyl, optionally substituted alkynyl, —CN, —OR^{1a}, optionally substituted aryl, and/or optionally substituted heteroaryl. In some embodiments, R³ is phenyl optionally substituted with halogen, optionally substituted alkyl, optionally substituted alkynyl, —CN, —NO₂, —OR^{1a}, optionally substituted phenyl, optionally substituted triazolyl, optionally substituted tetrazolyl, optionally substituted thiazolyl, or optionally substituted oxazolyl. In some embodiments, R³ is phenyl substituted with halogen, optionally substituted alkynyl, —CN, —OR^{1a}, optionally substituted phenyl, optionally substituted triazolyl, and/or optionally substituted tetrazolyl. In some embodiments, R³ is phenyl substituted with halogen, —OR^{1a}, and/or optionally substituted triazolyl.

In some embodiments, R³ is phenyl substituted with halogen, optionally substituted alkyl, optionally substituted alkenyl, optionally substituted alkynyl, —CN, —NO₂, —OR^{1a}, —N(R^{1a})₂, —NR^{1a}C(═O)R^{1a}, optionally substituted carbocyclyl, optionally substituted heterocyclyl, optionally substituted aryl, and/or optionally substituted heteroaryl. In some embodiments, R³ is phenyl substituted with halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, optionally substituted C₃₋₄ carbocyclyl, optionally substituted C₃₋₄ heterocyclyl, optionally substituted phenyl, optionally substituted 5-6 membered heteroaryl containing 1, 2, 3, or 4 ring heteroatoms selected from N, O, and S, and/or optionally substituted 8-10 membered heteroaryl containing 1, 2, 3, or 4 ring heteroatoms selected from N, O, and S.

In some embodiments, R³ is phenyl substituted with halogen. In some embodiments, R³ is phenyl substituted with —F, —Cl, —Br, or —I. In some embodiments, R³ is phenyl substituted with —F, —Cl, or —Br. In some embodiments, R³ is phenyl substituted with —F or —Cl. In some embodiments, R³ is phenyl substituted with —F. In some embodiments, R³ is phenyl substituted with —Cl. In some embodiments, R³ is phenyl substituted with —Br.

In some embodiments, R³ is phenyl substituted with optionally substituted alkyl. In some embodiments, R³ is phenyl substituted with optionally substituted C₁₋₆ alkyl. In some embodiments, R³ is phenyl substituted with optionally substituted C₁₋₃ alkyl. In some embodiments, R³ is phenyl substituted with substituted C₁₋₆ alkyl. In some embodiments, R³ is phenyl substituted with C₁₋₆ haloalkyl. In some embodiments, R³ is phenyl substituted with C₁ haloalkyl. In some embodiments, R³ is phenyl substituted with —CH₂F, —CHF₂, or —CF₃. In some embodiments, R³ is phenyl substituted with —CHF₂.

In some embodiments, R³ is phenyl substituted with optionally substituted alkynyl. In some embodiments, R³ is phenyl substituted with optionally substituted C₂₋₃ alkynyl. In some embodiments, R³ is phenyl substituted with optionally substituted C₂₋₆ alkynyl. In some embodiments, R³ is phenyl substituted with substituted C₂₋₆ alkynyl. In some embodiments, R³ is phenyl substituted with unsubstituted C₂₋₆ alkynyl. In some embodiments, R³ is phenyl substituted with optionally substituted ethynyl. In some embodiments, R³ is phenyl substituted with substituted ethynyl. In some embodiments, R³ is phenyl substituted with unsubstituted ethynyl. In some embodiments, R³ is phenyl substituted with —CN. In some embodiments, R³ is phenyl substituted with —OR^{1a}. In some embodiments, R³ is phenyl substituted with —O(optionally substituted alkyl). In some embodiments, R³ is phenyl substituted with —O(optionally substituted C₁₋₆ alkyl). In some embodiments, R³ is phenyl substituted with —O(substituted C₁₋₆ alkyl). In some embodiments, R³ is phenyl substituted with —O(optionally substituted C₁₋₃ alkyl). In some embodiments, R³ is phenyl substituted with —O(substituted C₁₋₆ haloalkyl). In some embodiments, R³ is phenyl substituted with —O(substituted C₁₋₆ perfluoroalkyl). In some embodiments, R³ is phenyl substituted with —OCF₃. In some embodiments, R³ is phenyl substituted with —O(unsubstituted C₁₋₆ alkyl). In some embodiments, R³ is phenyl substituted with —OCH₃. In some embodiments, R³ is phenyl substituted with —O(optionally substituted C₁₋₆ alkyl) and halogen. In some embodiments, R³ is phenyl substituted with —OCH₃ and —F or —Cl. In some embodiments, R³ is phenyl substituted with —N(R^{1a})₂. In some embodiments, R³ is phenyl substituted with —NR^{1a}C(═O)R^{1a}. In some embodiments, R³ is phenyl substituted with optionally substituted carbocyclyl. In some embodiments, R³ is phenyl substituted with optionally substituted C₃₋₄ carbocyclyl. In some embodiments, R³ is phenyl substituted with optionally substituted heterocyclyl. In some embodiments, R³ is phenyl substituted with optionally substituted C₃₋₄ heterocyclyl.

In some embodiments, R³ is phenyl substituted with —NO₂.

In some embodiments, R³ is phenyl substituted with optionally substituted phenyl. In some embodiments, R³ is phenyl substituted with substituted phenyl. In some embodiments, R³ is phenyl substituted with unsubstituted phenyl. In some embodiments, R³ is phenyl substituted with optionally substituted heteroaryl. In some embodiments, R³ is phenyl substituted with optionally substituted 5-6 membered heteroaryl containing 1, 2, 3, or 4 ring heteroatoms selected from N, O, and S. In some embodiments, R³ is phenyl substituted with optionally substituted 5-6 membered heteroaryl containing 1 ring S atom; 2 ring N atoms; 1 ring N atom and 1 ring O atom; 1 ring N atom and 1 ring S atom; 2 ring N atoms and 1 ring S atom; 3 ring N atoms; or 4 ring N atoms. In some embodiments, R³ is phenyl substituted with optionally substituted 8-10 membered heteroaryl containing 1, 2, 3, or 4 ring heteroatoms selected from N, O, and S. In some embodiments, R³ is phenyl substituted with optionally substituted 8-10 membered heteroaryl containing 2 ring N atoms; 1 ring N atom and 1 ring S atom; 2 ring N atoms and 1 ring S atom; 2 ring N atoms and 1 ring O atom; or 3 ring N atoms and 1 ring S atom.

In some embodiments, R³ is phenyl substituted with optionally substituted triazolyl. In some embodiments, R³ is phenyl substituted with substituted triazolyl. In some embodiments, R³ is phenyl substituted with triazolyl, wherein the triazolyl is substituted with halogen, optionally substituted alkyl, optionally substituted heterocyclyl, optionally substituted aryl, and/or optionally substituted heteroaryl. In some embodiments, R³ is phenyl substituted with triazolyl, wherein the triazolyl is substituted with —Cl, optionally substituted C₁₋₆ alkyl, optionally substituted 4-6 membered heterocyclyl, optionally substituted phenyl, or optionally substituted 5-6 membered heteroaryl. In some embodiments, R³ is phenyl substituted with triazolyl, wherein the triazolyl is substituted with —Cl. In some embodiments, R³ is phenyl substituted with triazolyl, wherein the triazolyl is substituted with substituted C₁₋₆ alkyl. In some embodiments, R³ is phenyl substituted with triazolyl, wherein the triazolyl is substituted with substituted methyl. In some embodiments, R³ is phenyl substituted with triazolyl, wherein the triazolyl is substituted with benzyl. In some embodiments, R³ is phenyl substituted with triazolyl, wherein the triazolyl is substituted with unsubstituted C₁₋₆ alkyl. In some embodiments, R³ is phenyl substituted with triazolyl, wherein the triazolyl is substituted with methyl.

In some embodiments, R³ is phenyl substituted with triazolyl, wherein the triazolyl is substituted with optionally substituted 4-6 membered heterocyclyl. In some embodiments, R³ is phenyl substituted with triazolyl, wherein the triazolyl is substituted with optionally substituted tetrahydropyranyl. In some embodiments, R³ is phenyl substituted with triazolyl, wherein the triazolyl is substituted with optionally substituted phenyl. In some embodiments, R³ is phenyl substituted with triazolyl, wherein the triazolyl is substituted with substituted phenyl. In some embodiments, R³ is phenyl substituted with triazolyl, wherein the triazolyl is substituted with unsubstituted phenyl. In some embodiments, R³ is phenyl substituted with triazolyl, wherein the triazolyl is substituted with optionally substituted 5-6 membered heteroaryl. In some embodiments, R³ is phenyl substituted with triazolyl, wherein the triazolyl is substituted with optionally substituted pyridyl. In some embodiments, R³ is phenyl substituted with unsubstituted triazolyl. In some embodiments, R³ is phenyl substituted with optionally substituted thiazolyl. In some embodiments, R³ is phenyl substituted with unsubstituted thiazolyl. In some embodiments, R³ is phenyl substituted with optionally substituted oxazolyl. R³ is phenyl substituted with unsubstituted oxazolyl. In some embodiments, R³ is phenyl substituted with optionally substituted tetrazolyl. In some embodiments, R³ is phenyl substituted with substituted tetrazolyl. In some embodiments, R³ is phenyl substituted with unsubstituted tetrazolyl.

In some embodiments, R³ is

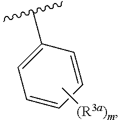

wherein:
each instance of R^{3a} is independently halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, at least one instance of R^{3a} is independently halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In some embodiments, at least one instance of R³, is halogen, optionally substituted alkyl, optionally substituted alkenyl, optionally substituted alkynyl, —CN, —OR^{1a}, optionally substituted aryl, or optionally substituted heteroaryl. In some embodiments, at least one instance of R^{3a} is halogen, optionally substituted alkyl, optionally substituted alkynyl, —CN, —NO₂, —OR^{1a}, optionally substituted phenyl, optionally substituted triazolyl, optionally substituted tetrazolyl, optionally substituted thiazolyl, or optionally substituted oxazolyl. In some embodiments, at least one instance of R^{3a} is halogen, optionally substituted alkynyl, —CN, —OR^{1a}, optionally substituted phenyl, optionally substituted triazolyl, or optionally substituted tetrazolyl. In some embodiments, at least one instance of R^{3a} is halogen, —OR^{1a}, or optionally substituted triazolyl. In some embodiments, at least one instance of R^{3a} is halogen, —OR^{1a}, optionally substituted triazolyl, optionally substituted thiazolyl, or optionally substituted oxazolyl.

In some embodiments, at least one instance of R^{3a} is —NO₂.

In some embodiments, at least one instance of R^{3a} is halogen, optionally substituted alkyl, optionally substituted alkenyl, optionally substituted alkynyl, —CN, —NO₂, —OR^{1a}, —N(R^{1a})₂, —NR^{1a}C(═O)R^{1a}, optionally substituted carbocyclyl, optionally substituted heterocyclyl, optionally substituted aryl, or optionally substituted heteroaryl. In some embodiments, at least one instance of R^{3a} is halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, optionally substituted C₃₋₄ carbocyclyl, optionally substituted C₃₋₄ heterocyclyl, optionally substituted phenyl, optionally substituted 5-6 membered heteroaryl containing 1, 2, 3, or 4 ring heteroatoms selected from N, O, and S, and/or optionally substituted 8-10 membered heteroaryl containing 1, 2, 3, or 4 ring heteroatoms selected from N, O, and S.

In some embodiments, at least one instance of R^{3a} is halogen. In some embodiments, at least one instance of R^{3a} is —F, —Cl, —Br, or —I. In some embodiments, at least one instance of R^{3a} is —F, —Cl, or —Br. In some embodiments, at least one instance of R^{3a} is —F or —Cl. In some embodiments, at least one instance of R^{3a} is —F. In some embodiments, at least one instance of R^{3a} is —Cl. In some embodiments, at least one instance of R^{3a} is —Br.

In some embodiments, R^{3a} is optionally substituted alkyl. In some embodiments, at least one instance of R^{3a} is optionally substituted C₁₋₃ alkyl. In some embodiments, R^{3a} is optionally substituted C₁₋₆ alkyl. In some embodiments, R^{3a} is substituted C₁₋₆ alkyl. In some embodiments, R^{3a} is C₁₋₆ haloalkyl. In some embodiments, R^{3a} is C₁ haloalkyl. In some embodiments, R^{3a} is —CH₂F, —CHF₂, or —CF₃. In some embodiments, R^{3a} is —CHF₂.

In some embodiments, at least one instance of R^{3a} is optionally substituted alkynyl. In some embodiments, at least one instance of R^{3a} is optionally substituted C₂₋₆ alkynyl. In some embodiments, at least one instance of R^{3a} is substituted C₂₋₆ alkynyl. In some embodiments, at least one instance of R^{3a} is unsubstituted C₂₋₆ alkynyl. In some embodiments, at least one instance of R^{3a} is optionally substituted ethynyl. In some embodiments, at least one instance of R^{3a} is substituted ethynyl. In some embodiments, at least one instance of R^{3a} is unsubstituted ethynyl. In some embodiments, at least one instance of R^{3a} is —CN. In some embodiments, at least one instance of R^{3a} is —NO₂. In some embodiments, at least one instance of R^{3a} is —OR^{1a}. In some embodiments, at least one instance of R^{3a} is —O(optionally substituted alkyl). In some embodiments, at least one instance of R^{3a} is —O(optionally substituted C₁₋₆ alkyl). In some embodiments, at least one instance of R^{3a} is —O(optionally substituted C₁₋₆ alkyl), and at least one instance of R^{3a} is halogen. In some embodiments, at least one instance of R^{3a} is —O(substituted C₁₋₆ alkyl). In some embodiments, at least one instance of R^{3a} is —O(unsubstituted C₁₋₆ alkyl). In some embodiments, at least one instance of R^{3a} is —O(optionally substituted C₁₋₃ alkyl). In some embodiments, at least one instance of R^{3a} is —OCH₃. In some embodiments, at least one instance of R^{3a} is —OCH₃, and at least one instance of R^{3a} is —F or —Cl. In some embodiments, R^{3a} is —O(substituted C₁₋₆ haloalkyl). In some embodiments, R^{3a} is —O(substituted C₁₋₆ perfluoroalkyl). In some embodiments, R^{3a}—OCF₃. In some embodiments, at least one instance of R^{3a} is —N(R^{1a})₂. In some embodiments, at least one instance of R^{3a} is —NR^{1a}C(═O)R^{1a}. In some embodiments, at least one instance of R^{3a} is optionally substituted carbocyclyl. In some embodiments, at least one instance of R^{3a} is optionally substituted C₃₋₄ carbocyclyl. In some embodiments, at least one instance of R^{3a} is optionally substituted heterocyclyl. In some embodiments, at least one instance of R^{3a} is optionally substituted C₃₋₄ heterocyclyl.

In some embodiments, at least one instance of R^{3a} is optionally substituted phenyl. In some embodiments, at least one instance of R^{3a} is substituted phenyl. In some embodiments, at least one instance of R^{3a} is unsubstituted phenyl. In some embodiments, at least one instance of R^{3a} is optionally substituted heteroaryl. In some embodiments, at least one instance of R^{3a} is optionally substituted 5-6 membered heteroaryl containing 1, 2, 3, or 4 ring heteroatoms selected from N, O, and S. In some embodiments, at least one instance of R^{3a} is optionally substituted 5-6 membered heteroaryl containing 1 ring S atom; 2 ring N atoms; 1 ring N atom and 1 ring O atom; 1 ring N atom and 1 ring S atom; 2 ring N atoms and 1 ring S atom; 3 ring N atoms; or 4 ring N atoms. In some embodiments, at least one instance of R^{3a} is optionally substituted 8-10 membered heteroaryl containing 1, 2, 3, or 4 ring heteroatoms selected from N, O, and S. In some embodiments, at least one instance of R^{3a} is optionally substituted 8-10 membered heteroaryl containing 2 ring N atoms; 1 ring N atom and 1 ring S atom; 2 ring N atoms and 1 ring S atom; 2 ring N atoms and 1 ring O atom; or 3 ring N atoms and 1 ring S atom.

In some embodiments, at least one instance of R^{ff} is optionally substituted triazolyl. In some embodiments, at least one instance of R^{3a} is substituted triazolyl. In some embodiments, at least one instance of R^{3a} is triazolyl, wherein the triazolyl is substituted with halogen, optionally substituted alkyl, optionally substituted heterocyclyl, optionally substituted aryl, and/or optionally substituted heteroaryl. In some embodiments, at least one instance of R^{3a} is triazolyl, wherein the triazolyl is substituted with —Cl, optionally substituted C₁₋₆ alkyl, optionally substituted 4-6 membered heterocyclyl, optionally substituted phenyl, or optionally substituted 5-6 membered heteroaryl. In some embodiments, at least one instance of R^{3a} is triazolyl, wherein the triazolyl is substituted with substituted C₁₋₆ alkyl. In some embodiments, at least one instance of R^{3a} is triazolyl, wherein the triazolyl is substituted with substituted methyl. In some embodiments, at least one instance of R^{3a} is triazolyl, wherein the triazolyl is substituted with benzyl. In some embodiments, at least one instance of R^{3a} is triazolyl, wherein the triazolyl is substituted with pyrimidinyl. In some embodiments, at least one instance of R^{3a} is triazolyl, wherein the triazolyl is substituted with 2-amino-pyrimidinyl. In some embodiments, at least one instance of R^{3a} is triazolyl, wherein the triazolyl is substituted with unsubstituted C₁₋₆ alkyl. In some embodiments, at least one instance of R^{3a} is triazolyl, wherein the triazolyl is substituted with methyl. In some embodiments, at least one instance of R^{3a} is triazolyl, wherein the triazolyl is substituted with optionally substituted 4-6 membered heterocyclyl. In some embodiments, at least one instance of R^{3a} is triazolyl, wherein the triazolyl is substituted with optionally substituted tetrahydropyranyl. In some embodiments, at least one instance of R^{3a} is triazolyl, wherein the triazolyl is substituted with optionally substituted phenyl. In some embodiments, at least one instance of R^{3a} is triazolyl, wherein the triazolyl is substituted with substituted phenyl. In some embodiments, at least one instance of R^{3a} is triazolyl, wherein the triazolyl is substituted with unsubstituted phenyl. In some embodiments, at least one instance of R^{3a} is triazolyl, wherein the triazolyl is substituted with optionally substituted 5-6 membered heteroaryl. In some embodiments, at least one instance of R^{3a} is triazolyl, wherein the triazolyl is substituted with optionally substituted pyridyl. In some embodiments, at least one instance of R^{3a} is unsubstituted triazolyl. In some embodiments, at least one instance of R^{3a} is optionally substituted tetrazolyl. In some embodiments, at least one instance of R^{3a} is substituted tetrazolyl. In some embodiments, at least one instance of R^{3a} is unsubstituted tetrazolyl. In some embodiments, R^{3a} is optionally substituted thiazolyl. In some embodiments, R^{3a} is unsubstituted thiazolyl. In some embodiments, R^{3a} is optionally substituted oxazolyl. R^{3a} is unsubstituted oxazolyl.

In some embodiments, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0, 1, or 2. In some embodiments, m is 0 or 1.

In some embodiments, R³ is,

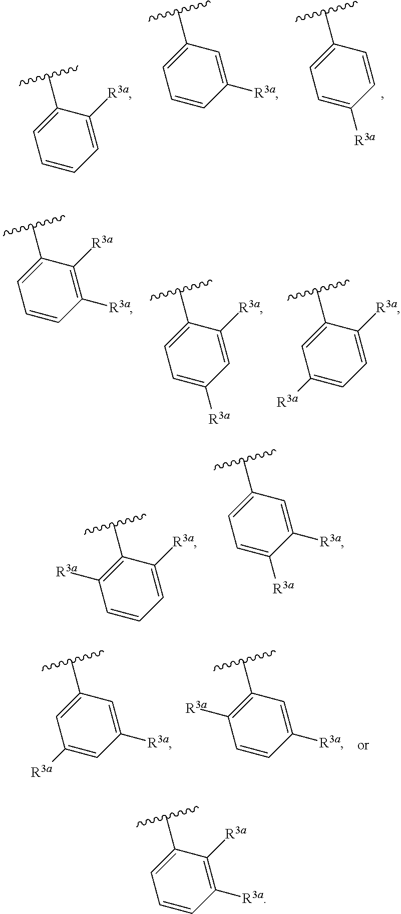

In some embodiments, R³ is

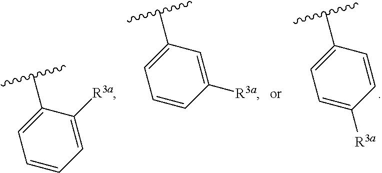

In some embodiments, R³ is

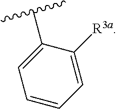

In some embodiments, R³ is

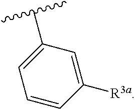

In some embodiments, R³ is

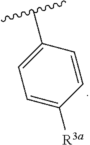

In some embodiments, R³ is

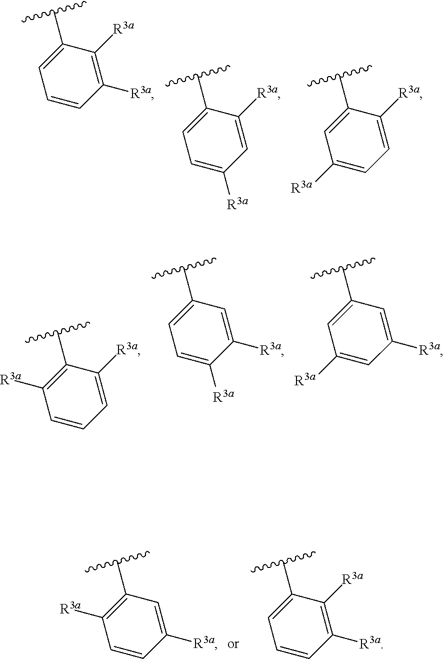

In some embodiments, R³ is

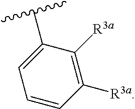

In some embodiments, R³ is

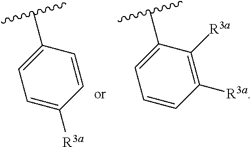

In some embodiments, R³ is

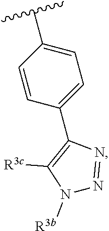

wherein R^{3b} is hydrogen, optionally substituted alkyl, optionally substituted heterocyclyl, optionally substituted aryl, or optionally substituted heteroaryl; and R^{3c} is hydrogen or halogen. In some embodiments, R^{3b} is hydrogen, methyl,

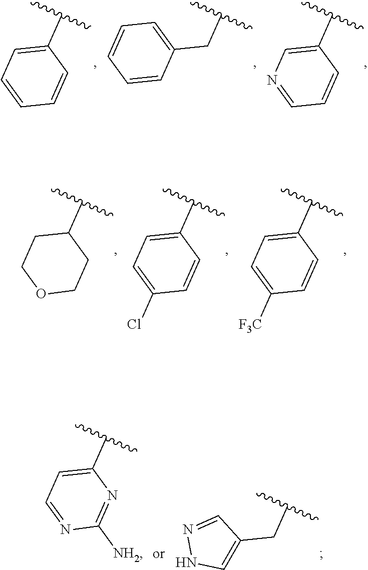

and R^{3c} is hydrogen or —Cl. In some embodiments, R^{3b} is hydrogen, methyl,

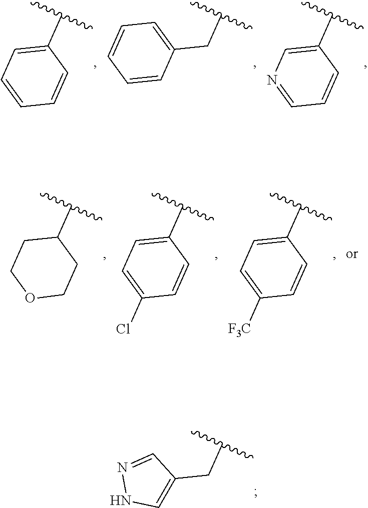

and R^{3c} is hydrogen or —Cl.

In some embodiments, R³ is

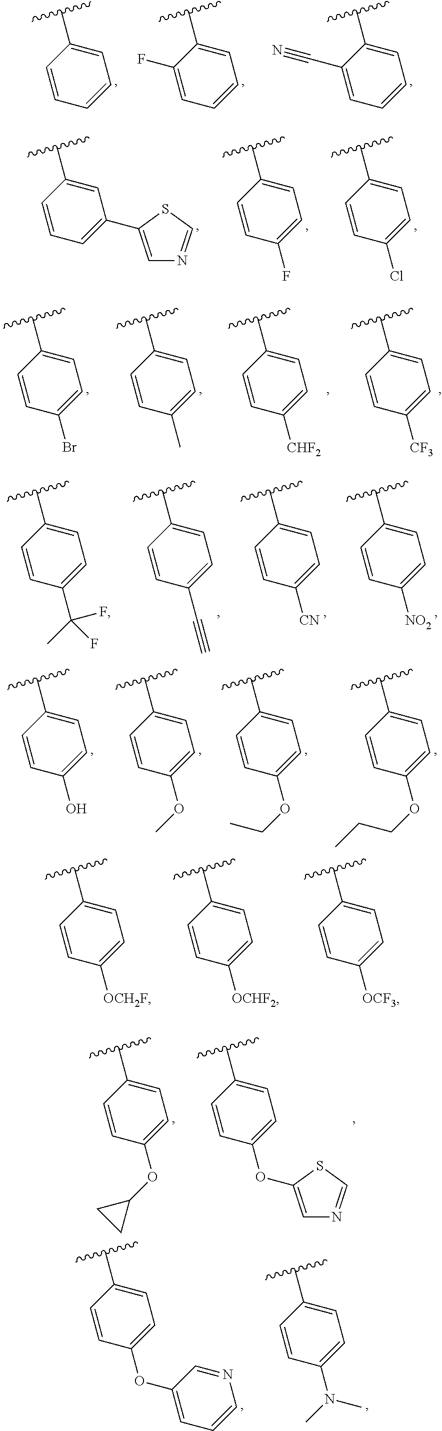

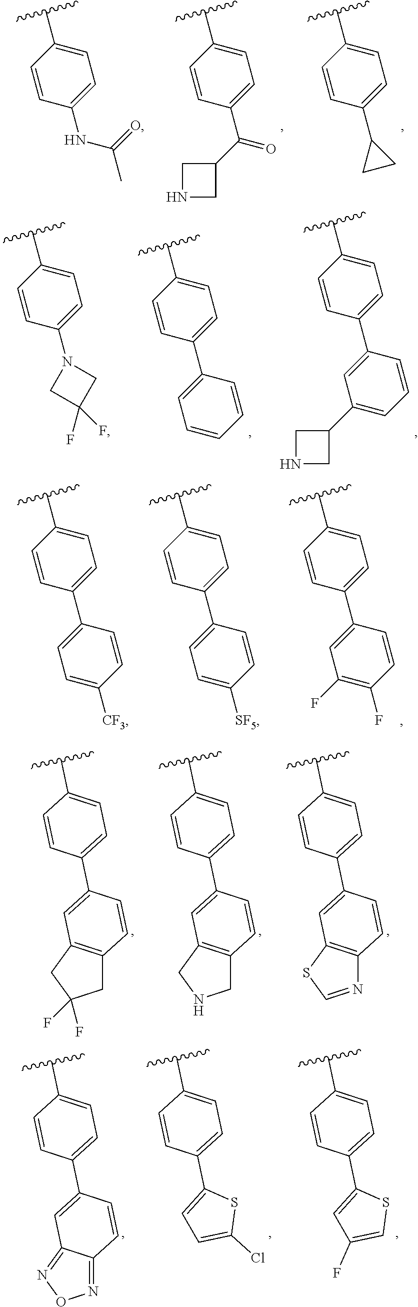

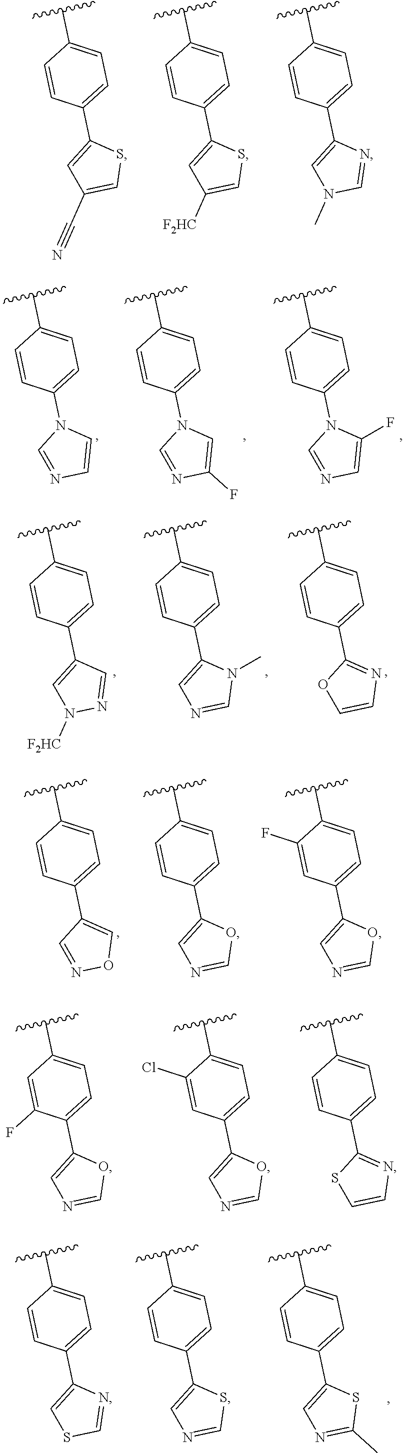

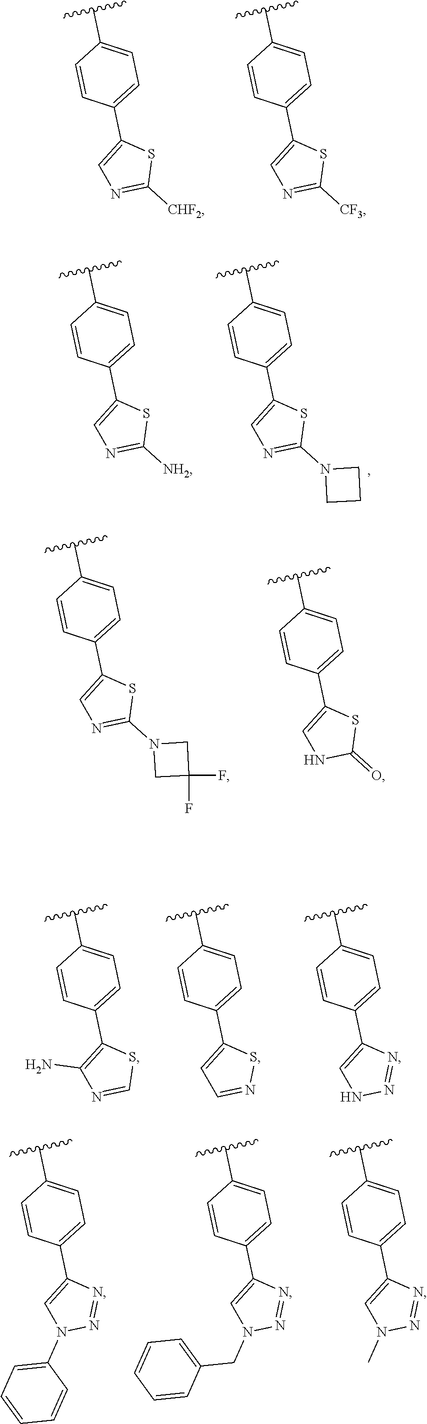

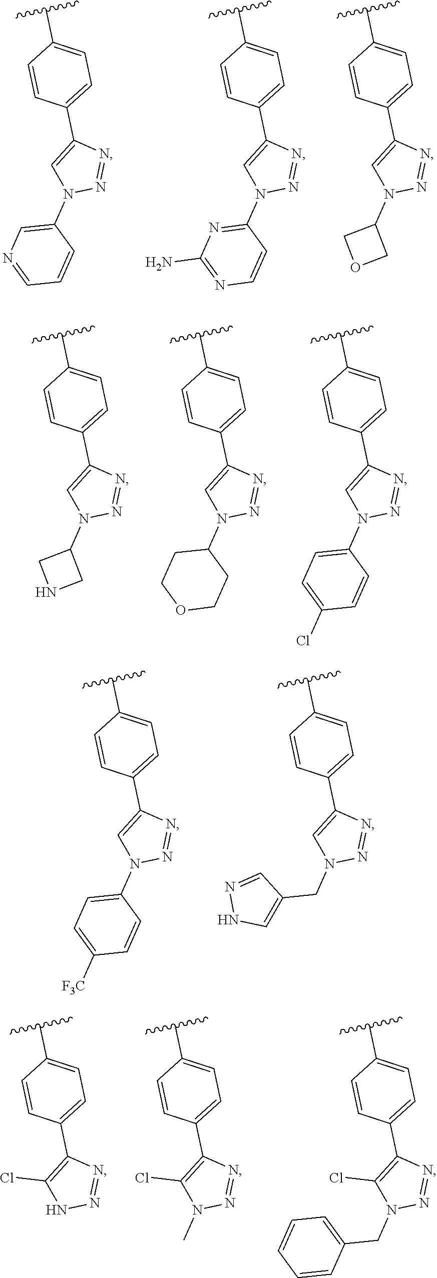

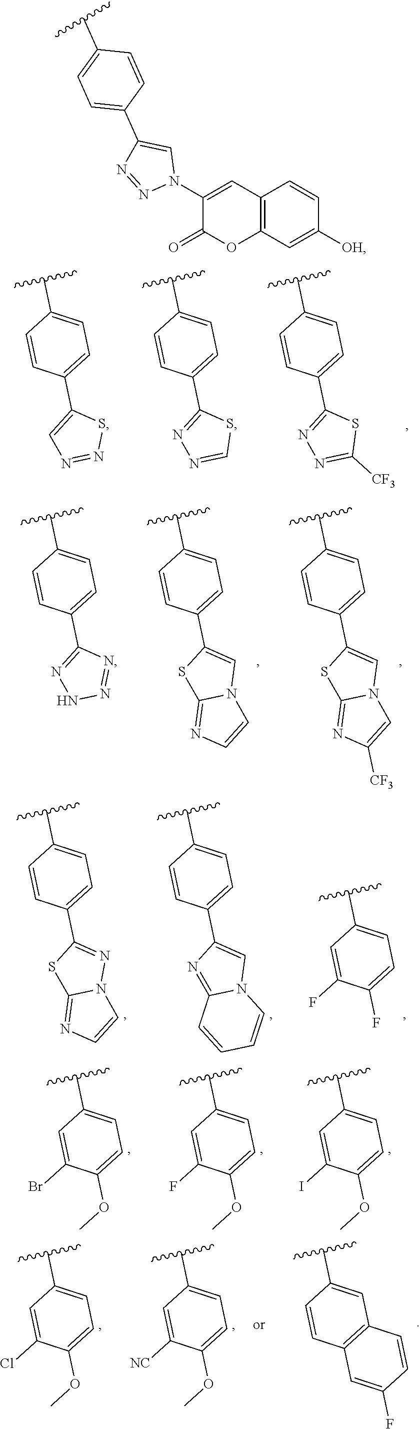

In some embodiments, R3 is

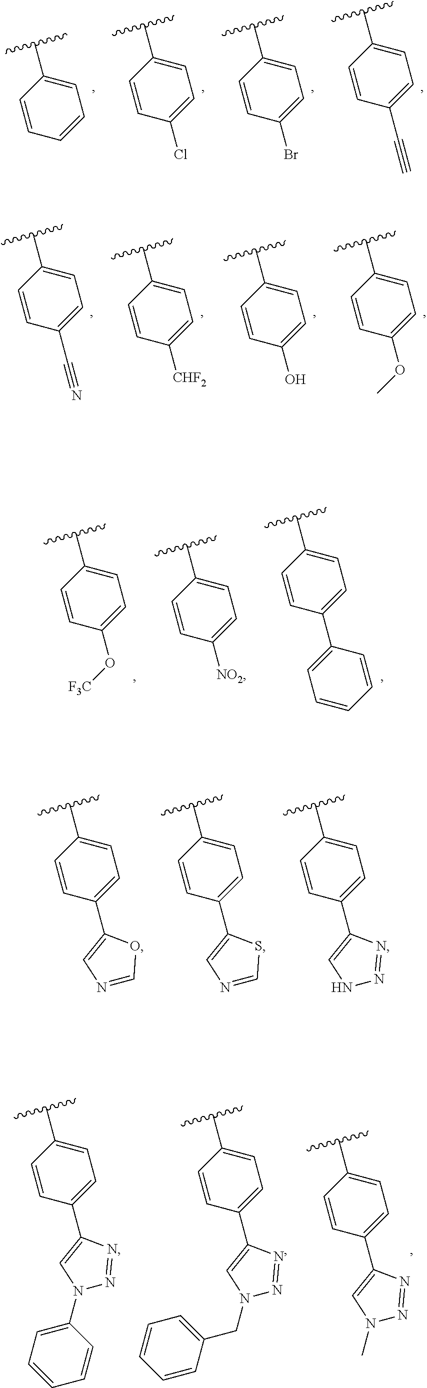

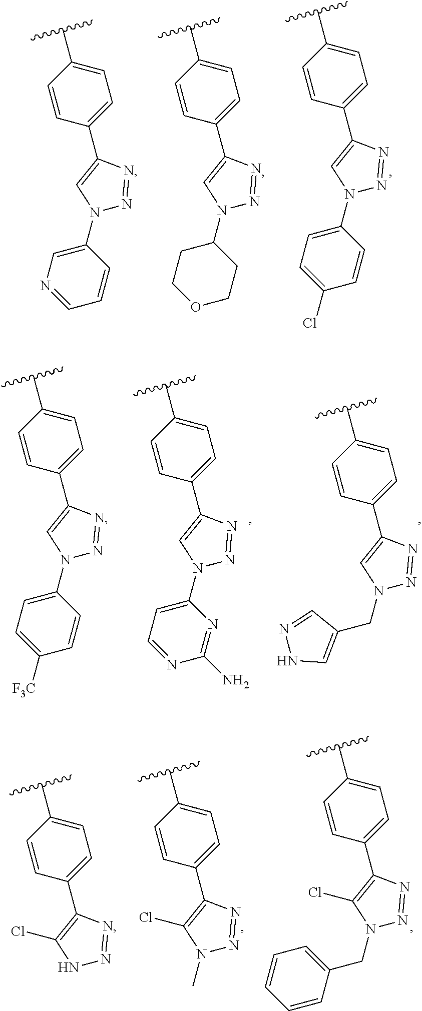

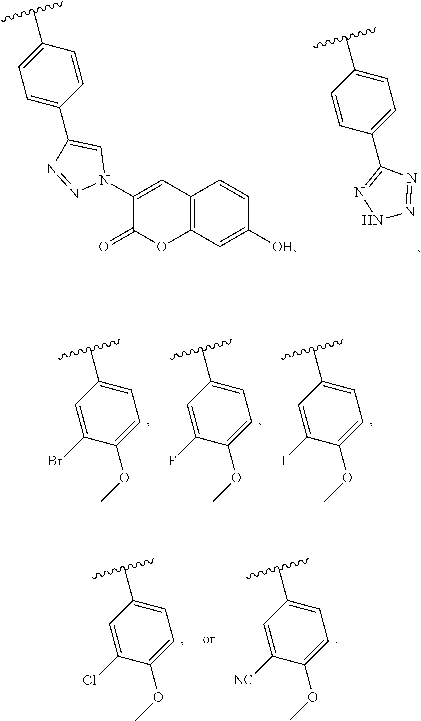

In some embodiments, R³ is

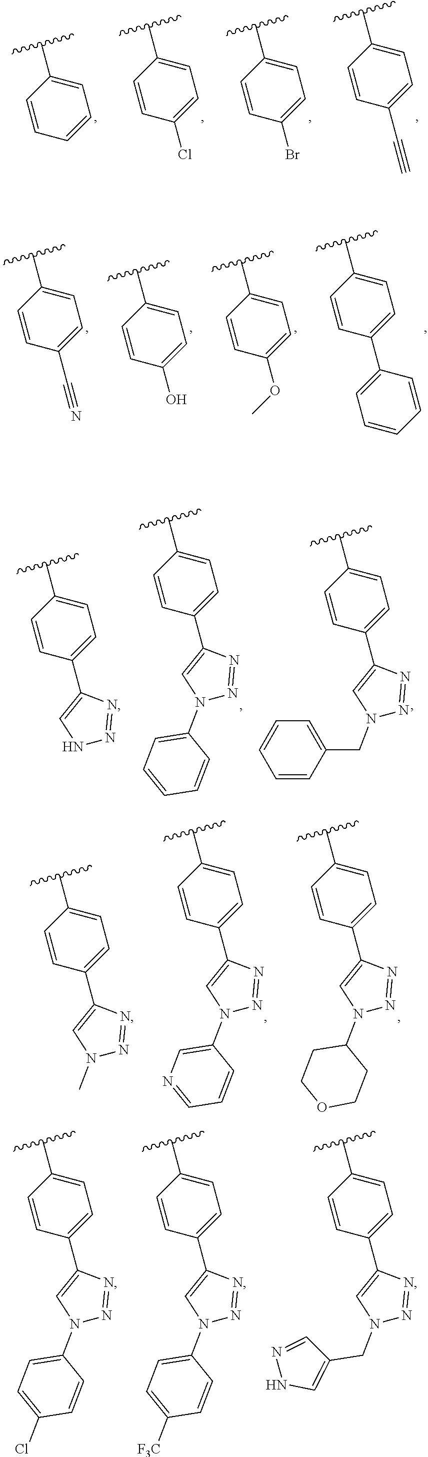

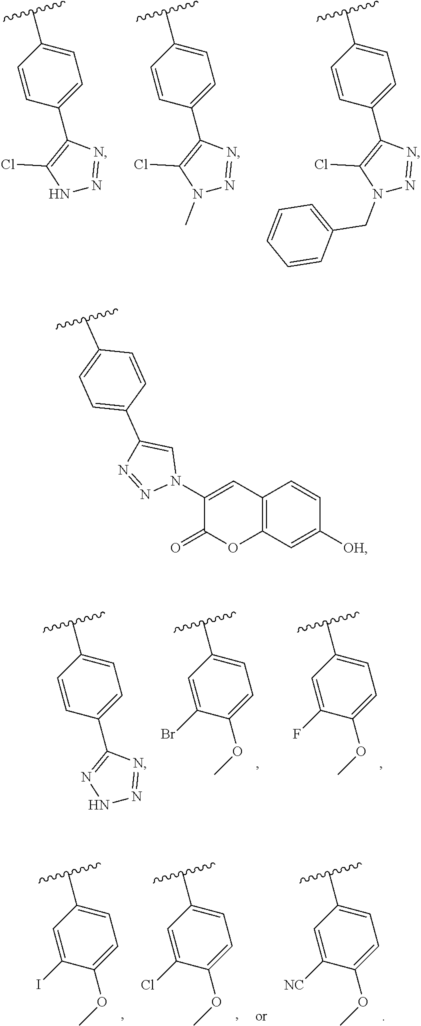

In some embodiments, R³ is

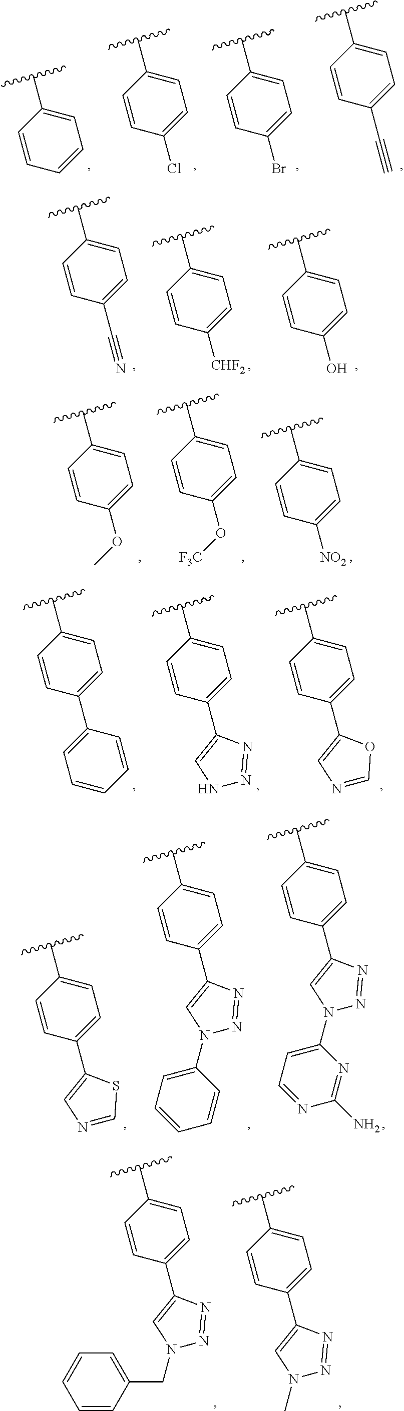

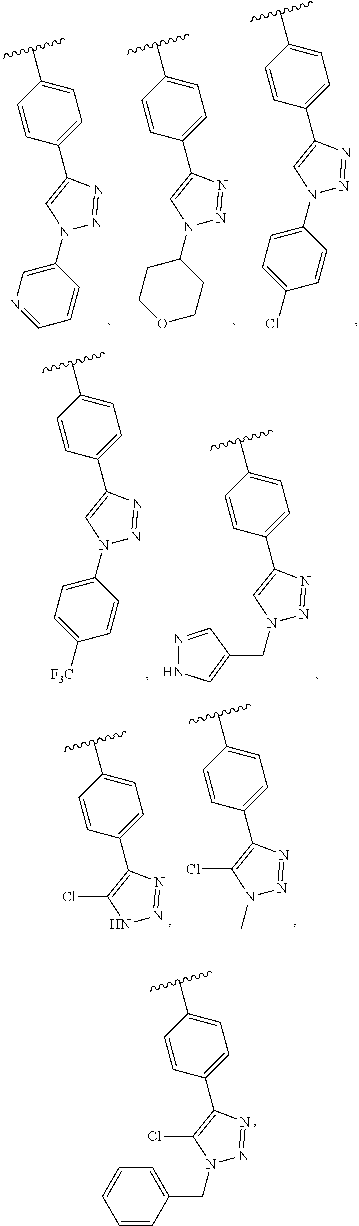

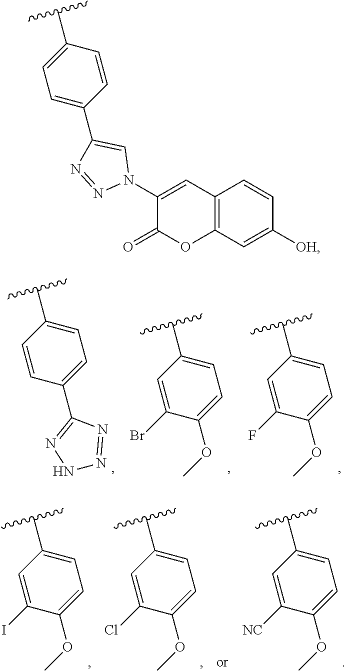

In some embodiments. R³ is

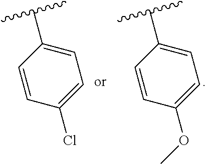

In some embodiments, R³ is

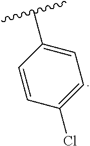 [SMILES: *c1ccc(Cl)cc1]

In some embodiments, R³ is

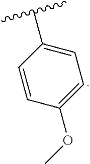 [SMILES: *c1ccc(OC)cc1]

In some embodiments, R³ is

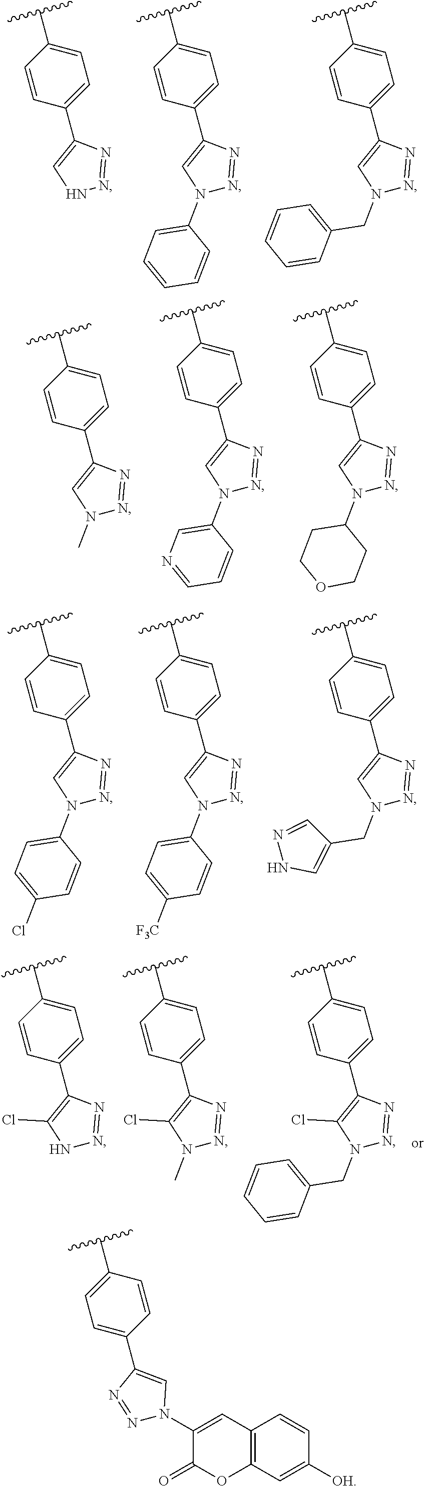

In some embodiments, R³ is

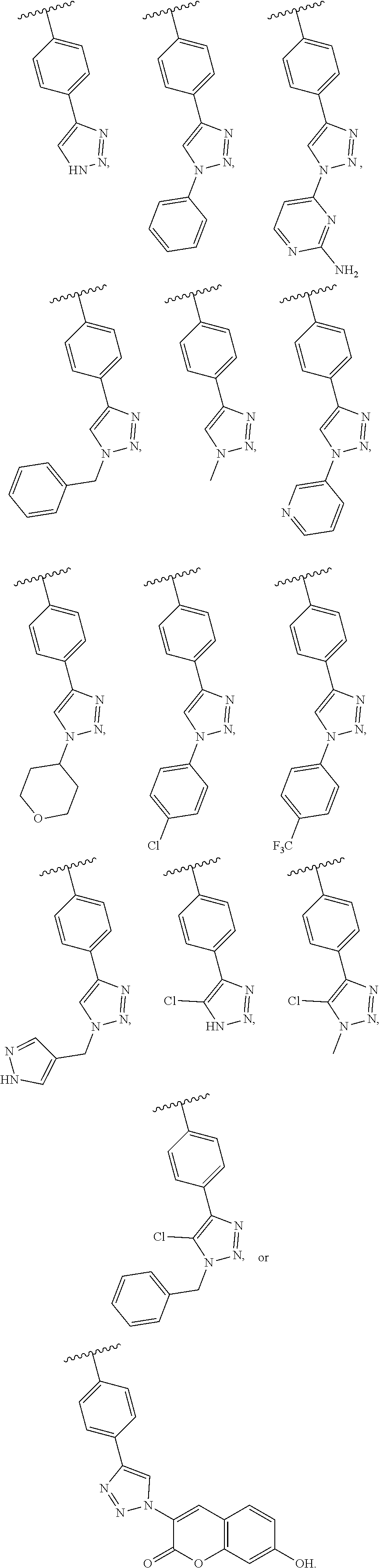

In some embodiments, R³ is

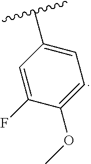 [SMILES: *c1ccc(OC)c(F)c1]

In some embodiments, R³ is

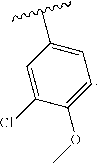 [SMILES: *c1ccc(OC)c(Cl)c1]

In some embodiments, R³ is

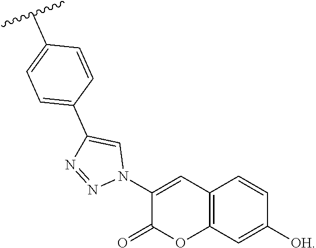 [SMILES: *c1ccc(-c2cn(-c3cc4ccc(O)cc4oc3=O)nn2)cc1]

In some embodiments, R³ is

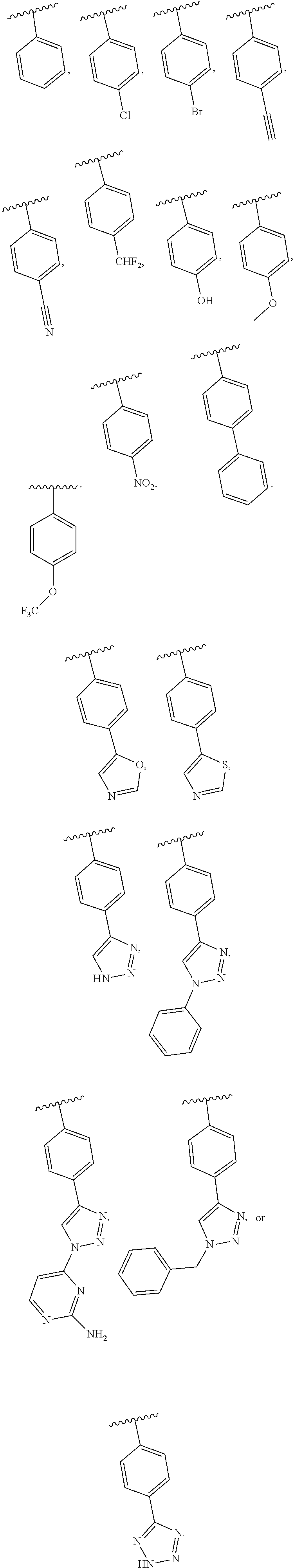

R⁴, R^{5a}, and R^{5b}

As generally described herein, R⁴ is hydrogen or optionally substituted alkyl. In some embodiments, R⁴ is hydrogen. In some embodiments, R⁴ is optionally substituted alkyl. In some embodiments, R⁴ is optionally substituted C₁₋₁₂ alkyl. In some embodiments, R⁴ is hydrogen or optionally substituted C₁₋₆ alkyl. In some embodiments, R⁴ is optionally substituted C₁₋₆ alkyl. In some embodiments, R⁴ is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R⁴ is substituted C₁₋₆ alkyl. In some embodiments, R⁴ is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R⁴ is hydrogen or unsubstituted C₁₋₆ alkyl. In some embodiments, R⁴ is unsubstituted C₁₋₆ alkyl. In some embodiments, R⁴ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R⁴ is hydrogen or optionally substituted methyl. In some embodiments, R⁴ is optionally substituted methyl. In some embodiments, R⁴ is substituted methyl. In some embodiments, R⁴ is methyl. In some embodiments, R⁴ is hydrogen or methyl.

As generally described herein, R^{5a} and R^{5b} are each hydrogen, or R^{5a} and R^{5b} are joined together with their intervening atom to form optionally substituted carbocyclyl. In some embodiments, R^{5a} and R^{5b} are each hydrogen. In some embodiments, R^{5a} and R^{5b} are joined together with their intervening atom to form optionally substituted carbocyclyl. In some embodiments, R^{5a} and R^{5b} are joined together with their intervening atom to form optionally substituted C₃₋₁₀ carbocyclyl. In some embodiments, R^{5a} and R^{5b} are joined together with their intervening atom to form optionally substituted C₃₋₆ carbocyclyl. In some embodiments, R^{5a} and R^{5b} are joined together with their intervening atom to form optionally substituted cyclopropyl, optionally substituted cyclobutyl, optionally substituted cyclopentyl, or optionally substituted cyclohexyl. In some embodiments, R^{5a} and R^{5b} are joined together with their intervening atom to form substituted cyclopropyl, substituted cyclobutyl, substituted cyclopentyl, or substituted cyclohexyl. In some embodiments, R^{5a} and R^{5b} are joined together with their intervening atom to form substituted cyclopropyl. In some embodiments, R^{5a} and R^{5b} are joined together with their intervening atom to form unsubstituted cyclopropyl, unsubstituted cyclobutyl, unsubstituted cyclopentyl, or unsubstituted cyclohexyl. In some embodiments, R^{5a} and R^{5b} are joined together with their intervening atom to form unsubstituted cyclopropyl. In some embodiments, R^{5a} and R^{5b} are each hydrogen, or R^{5a} and R^{5b} are joined together with their intervening atom to form optionally substituted cyclopropyl.

### R^{A}

As generally described herein, each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring.

In some embodiments, at least one instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring. In some embodiments, at least one instance of R^{A} is hydrogen. In some embodiments, at least one instance of R^{A} is optionally substituted acyl. In some embodiments, at least one instance of R^{A} is optionally substituted C₁₋₁₂ alkyl. In some embodiments, at least one instance of R^{A} is optionally substituted C₁₋₆ alkyl. In some embodiments, at least one instance of R^{A} is unsubstituted C₁₋₆ alkyl. In some embodiments, at least one instance of R^{A} is substituted C₁₋₆ alkyl. In some embodiments, at least one instance of R^{A} is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, at least one instance of R^{A} is optionally substituted C₂₋₁₂ alkenyl. In some embodiments, at least one instance of R^{A} is optionally substituted C₂₋₆ alkenyl. In some embodiments, at least one instance of R^{A} is optionally substituted ethenyl, optionally substituted 1-propenyl, optionally substituted 2-propenyl, optionally substituted 1-butenyl, optionally substituted 2-butenyl, optionally substituted butadienyl, optionally substituted pentenyl, optionally substituted pentadienyl, or optionally substituted hexenyl. In some embodiments, at least one instance of R^{A} is optionally substituted C₂₋₁₂ alkynyl. In some embodiments, at least one instance of R^{A} is optionally substituted C₂₋₆ alkynyl. In some embodiments, at least one instance of R^{A} is optionally substituted ethynyl, optionally substituted 1-propynyl, optionally substituted 2-propynyl, optionally substituted 1-butynyl, optionally substituted 2-butynyl, optionally substituted pentynyl, or optionally substituted hexynyl. In some embodiments, at least one instance of R^{A} is optionally substituted heteroC₁₋₁₂ alkyl. In some embodiments, at least one instance of R^{A} is optionally substituted heteroC₁₋₆ alkyl. In some embodiments, at least one instance of R^{A} is optionally substituted heteroC₁₋₁₂ alkenyl. In some embodiments, at least one instance of R^{A} is optionally substituted heteroC₁₋₆ alkenyl. In some embodiments, at least one instance of R^{A} is optionally substituted heteroC₁₋₁₂ alkynyl. In some embodiments, at least one instance of R^{A} is optionally substituted heteroC₁₋₆ alkynyl. In some embodiments, at least one instance of R^{A} is optionally substituted C₃₋₁₄ cycloalkyl. In some embodiments, at least one instance of R^{A} is optionally substituted 5-10 membered heterocyclyl. In some embodiments, at least one instance of R^{A} is optionally substituted 6-14 membered aryl. In some embodiments, at least one instance of R^{A} is optionally substituted 5-14 membered heteroaryl. In some embodiments, at least one instance of R^{A} is a nitrogen protecting group when attached to a nitrogen atom. In some embodiments, at least one instance of R^{A} is an oxygen protecting group when attached to an oxygen atom. In some embodiments, at least one instance of R^{A} is a sulfur protecting group when attached to a sulfur atom. In some embodiments, at least two instances of R^{A} are joined together with their intervening atom to form an optionally substituted 5-10 membered heterocyclic ring. In some embodiments, at least two instances of R^{A} are joined together with their intervening atom to form an optionally substituted 5-14 membered heteroaryl ring.

### Subgenera

In some embodiments, R¹ is optionally substituted heterocyclyl, optionally substituted aryl, or optionally substituted heteroaryl; R² is hydrogen; and n is 0 or 1. In some embodiments, R¹ is optionally substituted heterocyclyl, optionally substituted aryl, or optionally substituted heteroaryl; R² is hydrogen; and n is 0. In some embodiments, R¹ is optionally substituted heterocyclyl, optionally substituted aryl, or optionally substituted heteroaryl; R² is hydrogen; and n is 1.

In some embodiments, R¹ is optionally substituted 4-6 membered heterocyclyl, optionally substituted phenyl, or optionally substituted 5-6 membered heteroaryl; R² is hydrogen; and n is 0 or 1. In some embodiments, R¹ is optionally substituted 4-6 membered heterocyclyl, optionally substituted phenyl, or optionally substituted 5-6 membered heteroaryl; R² is hydrogen; and n is 0. In some embodiments, R¹ is optionally substituted 4-6 membered heterocyclyl, optionally substituted phenyl, or optionally substituted 5-6 membered heteroaryl; R² is hydrogen; and n is 1.

In some embodiments, R¹ is optionally substituted 4-6 membered heterocyclyl; R² is hydrogen; and n is 0 or 1. In some embodiments, R¹ is optionally substituted 4-6 membered heterocyclyl; R² is hydrogen; and n is 0. In some embodiments, R¹ is optionally substituted 4-6 membered heterocyclyl; R² is hydrogen; and n is 1.

In some embodiments, R¹ is optionally substituted phenyl; R² is hydrogen; and n is 0 or 1. In some embodiments, R¹ is optionally substituted phenyl; R² is hydrogen; and n is 0. In some embodiments, R¹ is optionally substituted phenyl; R² is hydrogen; and n is 1.

In some embodiments, R¹ is optionally substituted 5-6 membered heteroaryl; R² is hydrogen; and n is 0 or 1. In some embodiments, R¹ is optionally substituted 5-6 membered heteroaryl; R² is hydrogen; and n is 0. In some embodiments, R¹ is optionally substituted 5-6 membered heteroaryl; R² is hydrogen; and n is 1.

In some embodiments, R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydrothiopyranyl, optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted thiophenyl, optionally substituted oxazolyl, optionally substituted isoxazolyl, optionally substituted oxadiazolyl, optionally substituted thiazolyl, optionally substituted thiadiazolyl, or phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂; R² is hydrogen; and n is 0 or 1.

In some embodiments, R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, or optionally substituted tetrahydrothiopyranyl; R² is hydrogen; and n is 0 or 1. In some embodiments, R¹ is optionally substituted tetrahydropyranyl, optionally substituted azetidinyl, optionally substituted pyrrolidinyl, optionally substituted imidazolidinyl, optionally substituted piperazinyl, or optionally substituted tetrahydrothiopyranyl; R² is hydrogen; and n is 0 or 1. In some embodiments, R¹ is

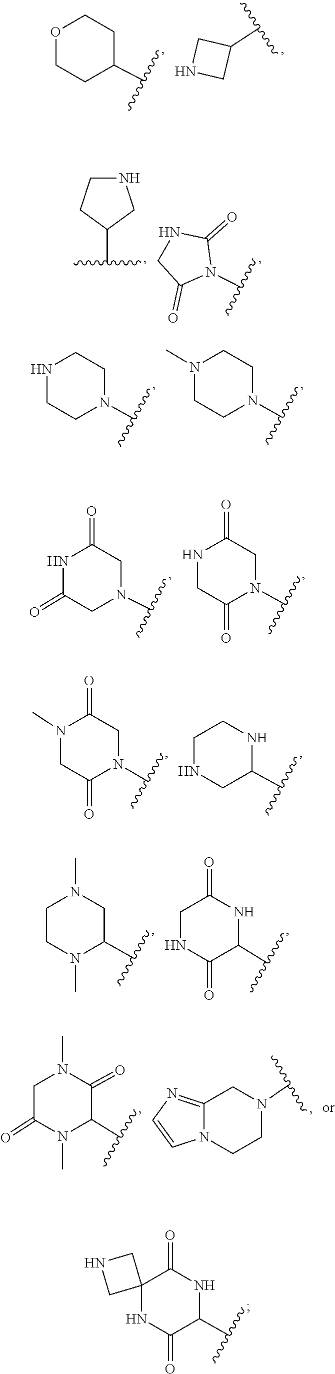

R² is hydrogen; and n is 0 or 1.

In some embodiments, R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted thiophenyl, optionally substituted oxazolyl, optionally substituted isoxazolyl, optionally substituted oxadiazolyl, optionally substituted thiazolyl, or optionally substituted thiadiazolyl; R² is hydrogen; and n is 0 or 1. In some embodiments, R¹ is optionally substituted pyridyl, optionally substituted pyrazinyl, optionally substituted pyrimidinyl, optionally substituted imidazolyl, optionally substituted triazolyl, optionally substituted tetrazolyl, optionally substituted thiophenyl, optionally substituted oxazolyl, optionally substituted isoxazolyl, optionally substituted oxadiazolyl, optionally substituted thiazolyl, optionally substituted thiadiazolyl; R² is hydrogen; and n is 0 or 1. In some embodiments, R¹ is

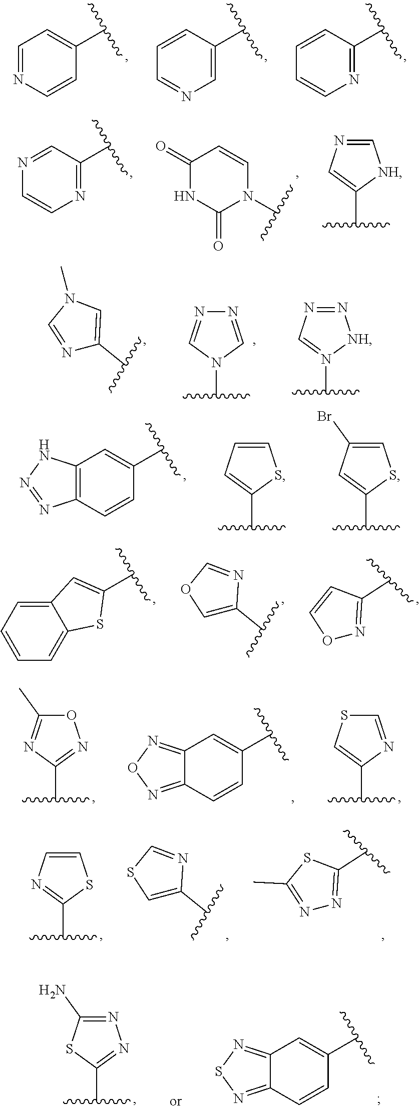

R² is hydrogen; and n is 0 or 1.

In some embodiments, R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂; R² is hydrogen; and n is 0 or 1. In some embodiments, R¹ is phenyl optionally substituted with halogen, optionally substituted alkyl, —OR^{1a}, and/or —N(R^{1a})₂; R² is hydrogen; and n is 0 or 1. In some embodiments, R¹ is

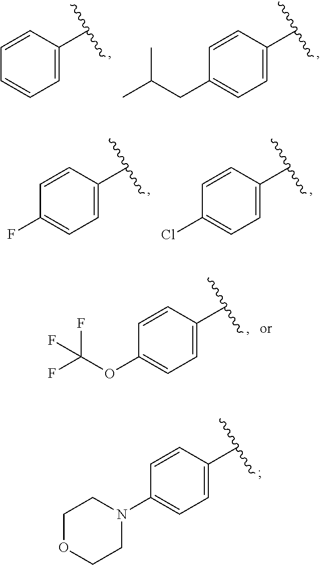 [SMILES: *c1ccccc1 ; *c1ccc(CC(C)C)cc1 ; *c1ccc(F)cc1 ; *c1ccc(Cl)cc1 ; *c1ccc(OC(F)(F)F)cc1 ; *c1ccc(N2CCOCC2)cc1]

R² is hydrogen; and n is 0 or 1.

In some embodiments, the compound of Formula (I) is of Formula (I-a-1), (I-a-2), (I-a-3), or (I-a-4):

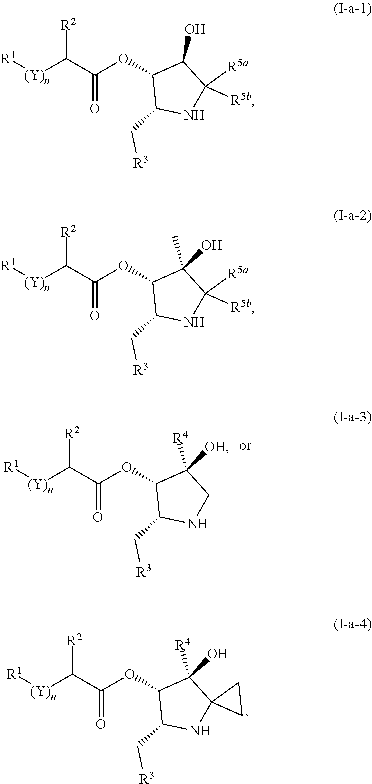

or a pharmaceutically acceptable salt thereof.

In some embodiments, the compound of Formula (I) is of Formula (I-b):

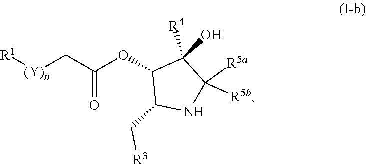

or a pharmaceutically acceptable salt thereof.

In some embodiments, the compound of Formula (I) is of Formula (I-b-1), (I-b-2), (I-b-3), or (I-b-4):

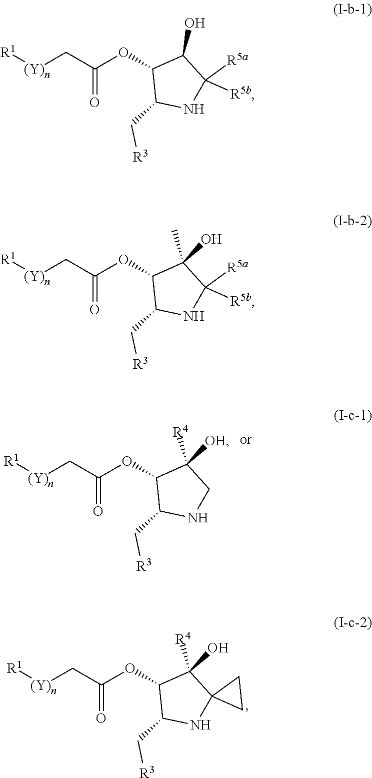

or a pharmaceutically acceptable salt thereof.

In some embodiments, the compound of Formula (I) is of Formula (I-c):

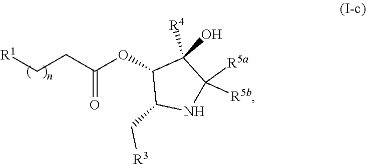

or a pharmaceutically acceptable salt thereof.

In some embodiments, the compound of Formula (I) is of Formula (I-c-1) or (I-c-2):

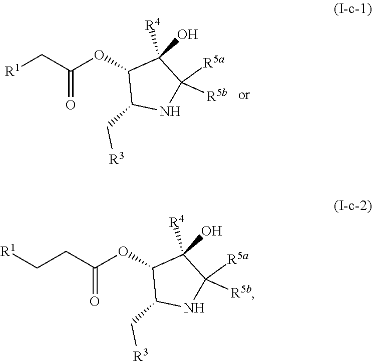

or a pharmaceutically acceptable salt thereof.

In some embodiments, the compound of Formula (I) is of Formula (II):

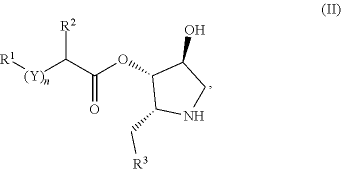

or a pharmaceutically acceptable salt thereof.

In some embodiments, the compound of Formula (II) is of Formula (II-a):

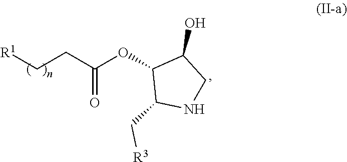

or a pharmaceutically acceptable salt thereof.

In some embodiments, the compound of Formula (II) is of Formula (II-a-1) or (II-a-2):

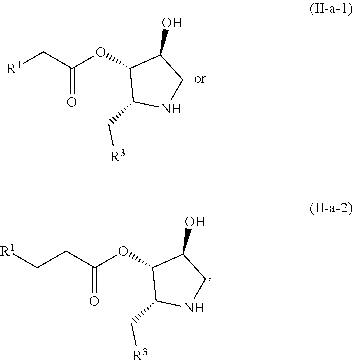

or a pharmaceutically acceptable salt thereof.

In some embodiments, the compound of Formula (I) is of Formula (III):

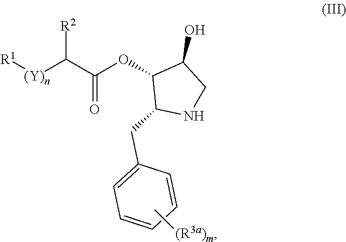

or a pharmaceutically acceptable salt thereof, wherein:
each instance of R^{3a} is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, the compound of Formula (III) is of Formula (III-a-1), (III-a-2), (III-a-3), (III-a-4), or (III-a-5):

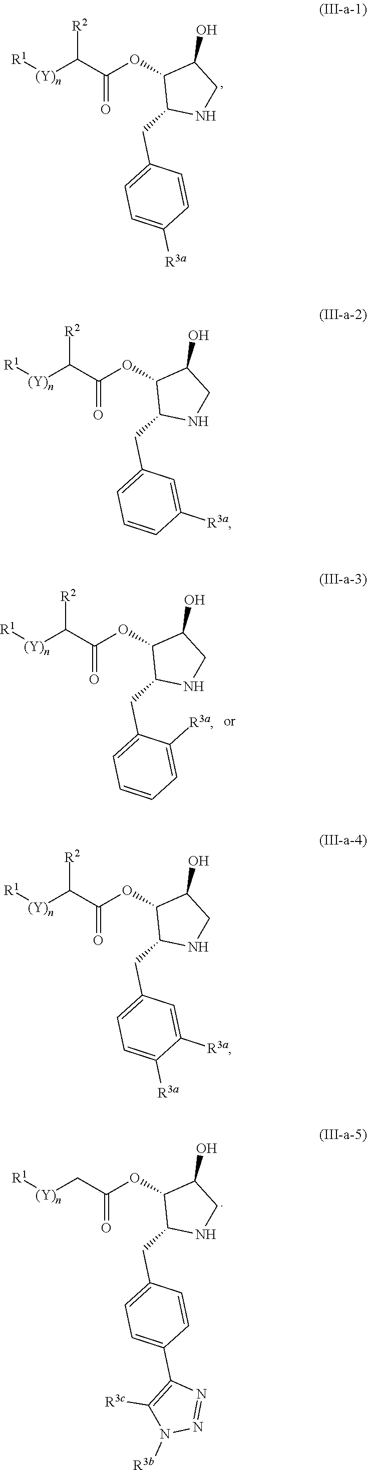

or a pharmaceutically acceptable salt thereof, wherein R^{3b} is hydrogen, optionally substituted alkyl, optionally substituted heterocyclyl, optionally substituted aryl, or optionally substituted heteroaryl; and R^{3c} is hydrogen or halogen.

In some embodiments, the compound of Formula (III) is of Formula (III-b):

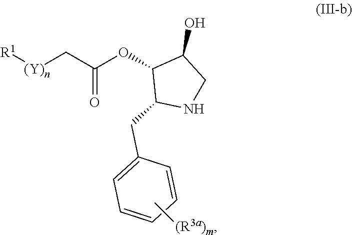

or a pharmaceutically acceptable salt thereof.

In some embodiments, the compound of Formula (III) is of Formula (III-b-1), (III-b-2), (III-b-3), (III-b-4), or (III-b-5):

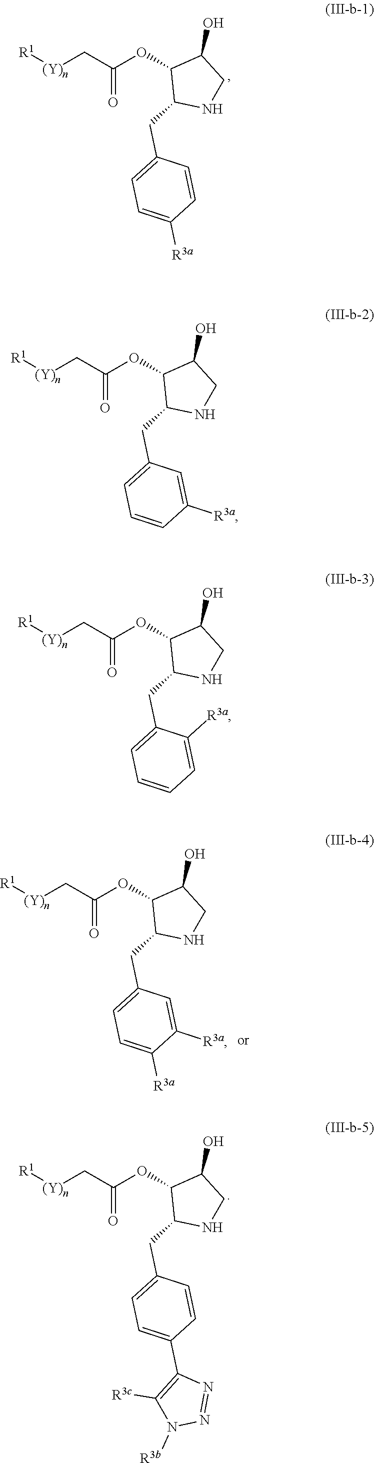

or a pharmaceutically acceptable salt thereof, wherein R^{3b} is hydrogen, optionally substituted alkyl, optionally substituted heterocyclyl, optionally substituted aryl, or optionally substituted heteroaryl; and R^{3c} is hydrogen or halogen.

In some embodiments, the compound of Formula (III) is of Formula (III-c):

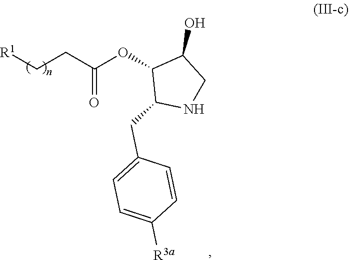

or a pharmaceutically acceptable salt thereof.

In some embodiments, the compound of Formula (III) is of Formula (III-c-1) or (III-c-2):

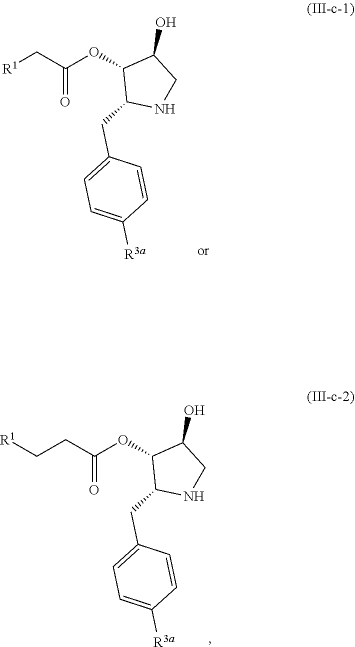

In some embodiments, the compound of Formula (I) is selected from those in Table 1A, and pharmaceutically acceptable salts thereof.

TABLE 1A
Compounds of Formula (I)

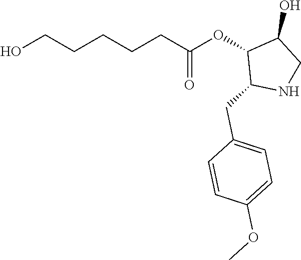 [SMILES: COc1ccc(C[C@H]2NC[C@H](O)[C@H]2OC(=O)CCCCCO)cc1]

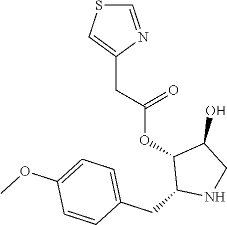 [SMILES: COc1ccc(C[C@H]2NC[C@H](O)[C@H]2OC(=O)Cc2cscn2)cc1]

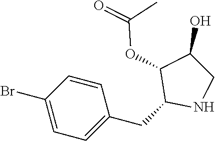 [SMILES: CC(=O)O[C@@H]1[C@@H](O)CN[C@@H]1Cc1ccc(Br)cc1]

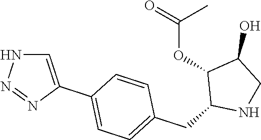 [SMILES: CC(=O)O[C@@H]1[C@@H](O)CN[C@@H]1Cc1ccc(-c2c[nH]nn2)cc1]

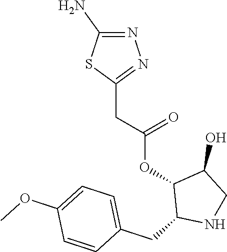 [SMILES: COc1ccc(C[C@H]2NC[C@H](O)[C@H]2OC(=O)Cc2nnc(N)s2)cc1]

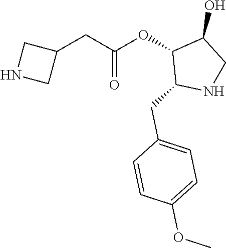 [SMILES: COc1ccc(C[C@H]2NC[C@H](O)[C@H]2OC(=O)CC2CNC2)cc1]

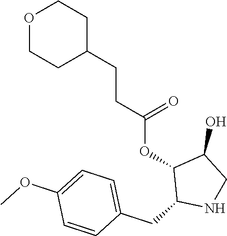 [SMILES: COc1ccc(C[C@H]2NC[C@H](O)[C@H]2OC(=O)CCC2CCOCC2)cc1]

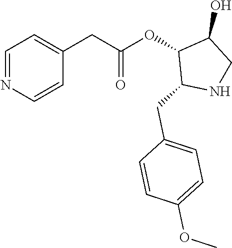 [SMILES: COc1ccc(C[C@H]2NC[C@H](O)[C@H]2OC(=O)Cc2ccncc2)cc1]

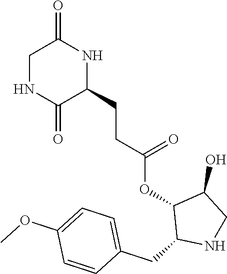 [SMILES: COc1ccc(C[C@H]2NC[C@H](O)[C@H]2OC(=O)CC[C@@H]2NC(=O)CNC2=O)cc1]

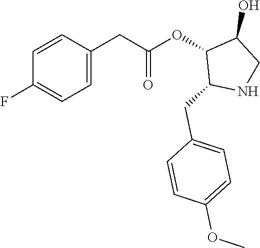 [SMILES: COc1ccc(C[C@H]2NC[C@H](O)[C@H]2OC(=O)Cc2ccc(F)cc2)cc1]

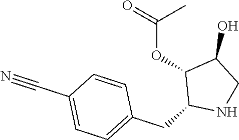 [SMILES: CC(=O)O[C@@H]1[C@@H](O)CN[C@@H]1Cc1ccc(C#N)cc1]

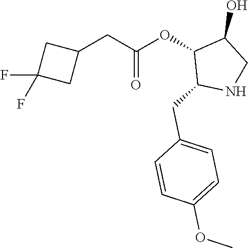 [SMILES: COc1ccc(C[C@H]2NC[C@H](O)[C@H]2OC(=O)CC2CC(F)(F)C2)cc1]

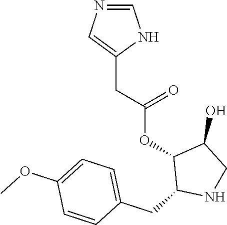 [SMILES: COc1ccc(C[C@H]2NC[C@H](O)[C@H]2OC(=O)Cc2cnc[nH]2)cc1]

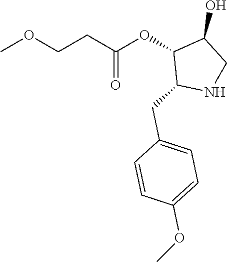 [SMILES: COCCC(=O)O[C@@H]1[C@@H](O)CN[C@@H]1Cc1ccc(OC)cc1]

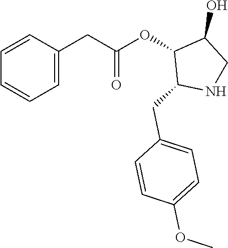 [SMILES: COc1ccc(C[C@H]2NC[C@H](O)[C@H]2OC(=O)Cc2ccccc2)cc1]

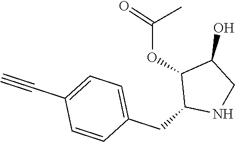 [SMILES: C#Cc1ccc(C[C@H]2NC[C@H](O)[C@H]2OC(C)=O)cc1]

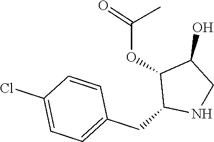 [SMILES: CC(=O)O[C@@H]1[C@@H](O)CN[C@@H]1Cc1ccc(Cl)cc1]

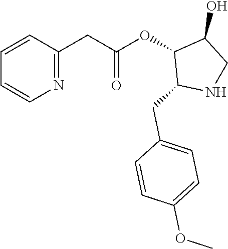 [SMILES: COc1ccc(C[C@H]2NC[C@H](O)[C@H]2OC(=O)Cc2ccccn2)cc1]

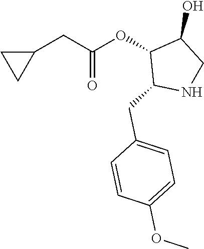 [SMILES: COc1ccc(C[C@H]2NC[C@H](O)[C@H]2OC(=O)CC2CC2)cc1]

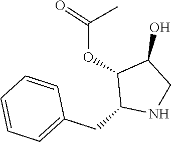 [SMILES: CC(=O)O[C@@H]1[C@@H](O)CN[C@@H]1Cc1ccccc1]

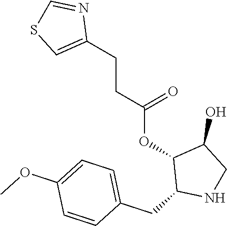 [SMILES: COc1ccc(C[C@H]2NC[C@H](O)[C@H]2OC(=O)CCc2cscn2)cc1]

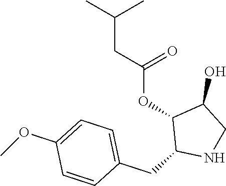 [SMILES: COc1ccc(C[C@H]2NC[C@H](O)[C@H]2OC(=O)CC(C)C)cc1]

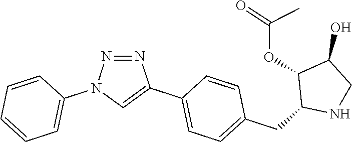 [SMILES: CC(=O)O[C@@H]1[C@@H](O)CN[C@@H]1Cc1ccc(-c2cn(-c3ccccc3)nn2)cc1]

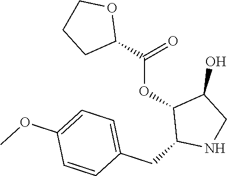 [SMILES: COc1ccc(C[C@H]2NC[C@H](O)[C@H]2OC(=O)[C@@H]2CCCO2)cc1]

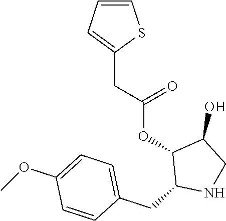 [SMILES: COc1ccc(C[C@H]2NC[C@H](O)[C@H]2OC(=O)Cc2cccs2)cc1]

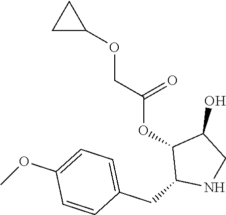 [SMILES: COc1ccc(C[C@H]2NC[C@H](O)[C@H]2OC(=O)COC2CC2)cc1]

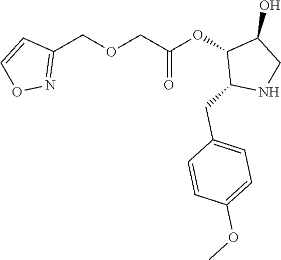 [SMILES: COc1ccc(C[C@H]2NC[C@H](O)[C@H]2OC(=O)COCc2ccon2)cc1]

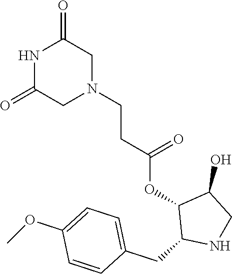 [SMILES: COc1ccc(C[C@H]2NC[C@H](O)[C@H]2OC(=O)CCN2CC(=O)NC(=O)C2)cc1]

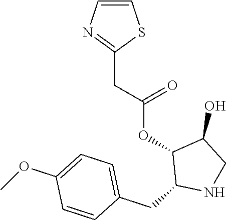 [SMILES: COc1ccc(C[C@H]2NC[C@H](O)[C@H]2OC(=O)Cc2nccs2)cc1]

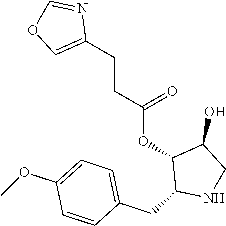 [SMILES: COc1ccc(C[C@H]2NC[C@H](O)[C@H]2OC(=O)CCc2cocn2)cc1]

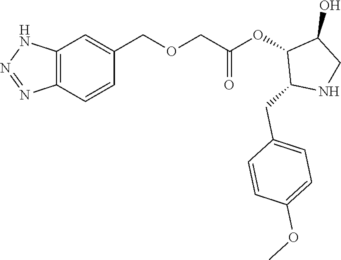 [SMILES: COc1ccc(C[C@H]2NC[C@H](O)[C@H]2OC(=O)COCc2ccc3nn[nH]c3c2)cc1]

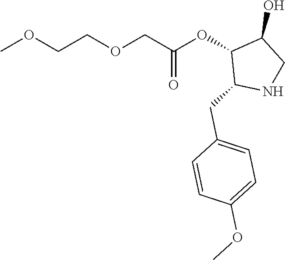 [SMILES: COCCOCC(=O)O[C@@H]1[C@@H](O)CN[C@@H]1Cc1ccc(OC)cc1]

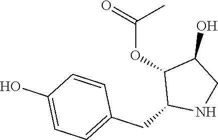 [SMILES: CC(=O)O[C@@H]1[C@@H](O)CN[C@@H]1Cc1ccc(O)cc1]

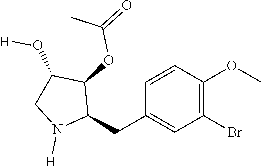 [SMILES: COc1ccc(C[C@H]2NC[C@H](O)[C@H]2OC(C)=O)cc1Br]

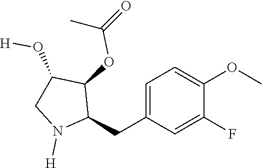 [SMILES: COc1ccc(C[C@H]2NC[C@H](O)[C@H]2OC(C)=O)cc1F]

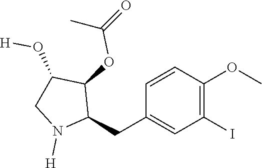 [SMILES: COc1ccc(C[C@H]2NC[C@H](O)[C@H]2OC(C)=O)cc1I]

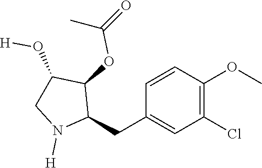 [SMILES: COc1ccc(C[C@H]2NC[C@H](O)[C@H]2OC(C)=O)cc1Cl]

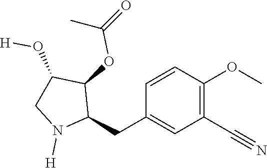 [SMILES: COc1ccc(C[C@H]2NC[C@H](O)[C@H]2OC(C)=O)cc1C#N]

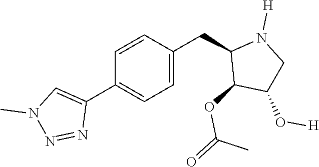

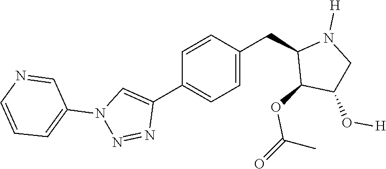 [SMILES: CC(=O)O[C@@H]1[C@@H](O)CN[C@@H]1Cc1ccc(-c2cn(-c3cccnc3)nn2)cc1]

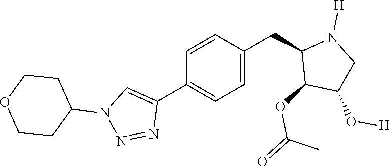 [SMILES: CC(=O)O[C@@H]1[C@@H](O)CN[C@@H]1Cc1ccc(-c2cn(C3CCOCC3)nn2)cc1]

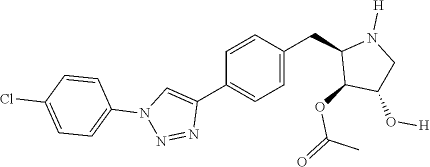

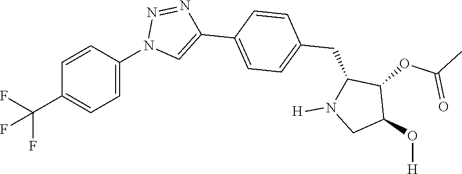 [SMILES: CC(=O)O[C@@H]1[C@@H](O)CN[C@@H]1Cc1ccc(-c2cn(-c3ccc(C(F)(F)F)cc3)nn2)cc1]

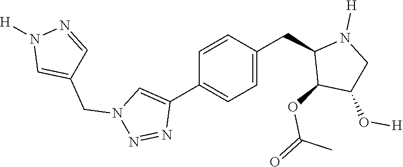

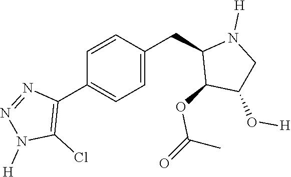

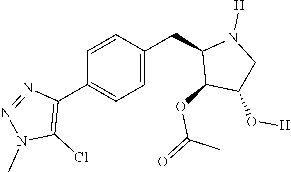

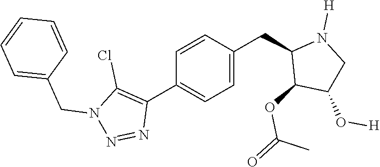

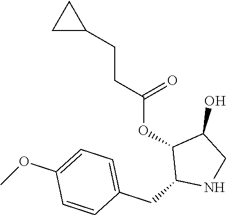 [SMILES: COc1ccc(C[C@H]2NC[C@H](O)[C@H]2OC(=O)CCC2CC2)cc1]

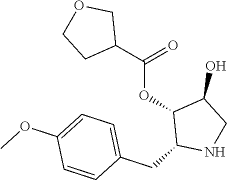 [SMILES: COc1ccc(C[C@H]2NC[C@H](O)[C@H]2OC(=O)C2CCOC2)cc1]

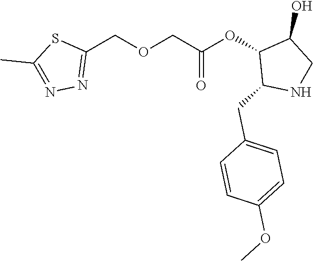 [SMILES: COc1ccc(C[C@H]2NC[C@H](O)[C@H]2OC(=O)COCc2nnc(C)s2)cc1]

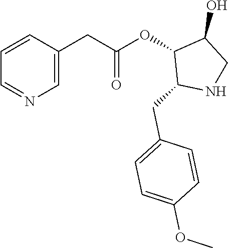 [SMILES: COc1ccc(C[C@H]2NC[C@H](O)[C@H]2OC(=O)Cc2cccnc2)cc1]

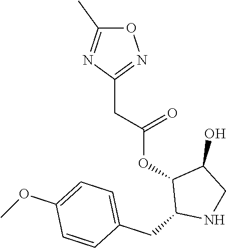 [SMILES: COc1ccc(C[C@H]2NC[C@H](O)[C@H]2OC(=O)Cc2noc(C)n2)cc1]

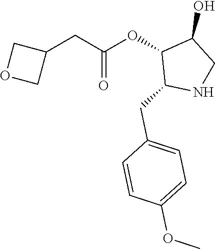 [SMILES: COc1ccc(C[C@H]2NC[C@H](O)[C@H]2OC(=O)CC2COC2)cc1]

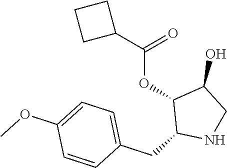 [SMILES: COc1ccc(C[C@H]2NC[C@H](O)[C@H]2OC(=O)C2CCC2)cc1]

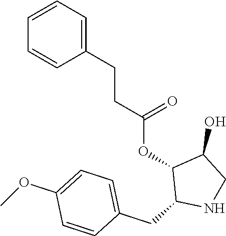 [SMILES: COc1ccc(C[C@H]2NC[C@H](O)[C@H]2OC(=O)CCc2ccccc2)cc1]

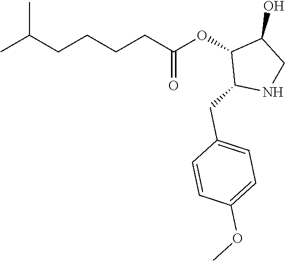 [SMILES: COc1ccc(C[C@H]2NC[C@H](O)[C@H]2OC(=O)CCCCC(C)C)cc1]

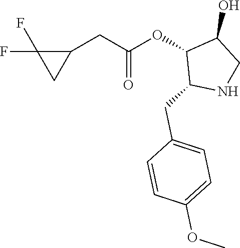 [SMILES: COc1ccc(C[C@H]2NC[C@H](O)[C@H]2OC(=O)CC2CC2(F)F)cc1]

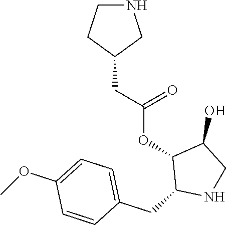 [SMILES: COc1ccc(C[C@H]2NC[C@H](O)[C@H]2OC(=O)C[C@@H]2CCNC2)cc1]

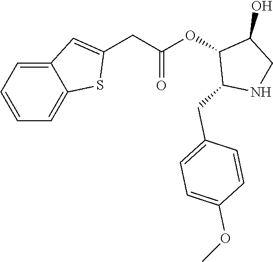 [SMILES: COc1ccc(C[C@H]2NC[C@H](O)[C@H]2OC(=O)Cc2cc3ccccc3s2)cc1]

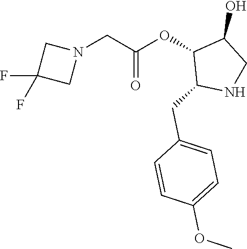 [SMILES: COc1ccc(C[C@H]2NC[C@H](O)[C@H]2OC(=O)CN2CC(F)(F)C2)cc1]

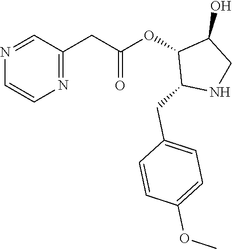 [SMILES: COc1ccc(C[C@H]2NC[C@H](O)[C@H]2OC(=O)Cc2cnccn2)cc1]

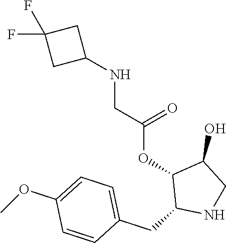 [SMILES: COc1ccc(C[C@H]2NC[C@H](O)[C@H]2OC(=O)CNC2CC(F)(F)C2)cc1]

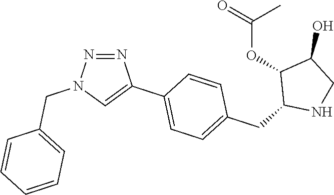 [SMILES: CC(=O)O[C@@H]1[C@@H](O)CN[C@@H]1Cc1ccc(-c2cn(Cc3ccccc3)nn2)cc1]

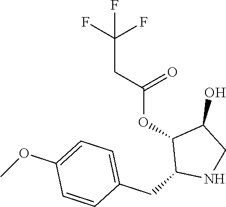 [SMILES: COc1ccc(C[C@H]2NC[C@H](O)[C@H]2OC(=O)CC(F)(F)F)cc1]

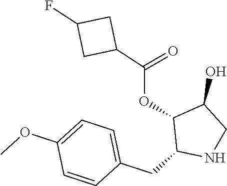 [SMILES: COc1ccc(C[C@H]2NC[C@H](O)[C@H]2OC(=O)C2CC(F)C2)cc1]

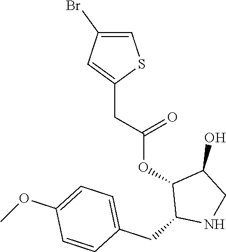 [SMILES: COc1ccc(C[C@H]2NC[C@H](O)[C@H]2OC(=O)Cc2cc(Br)cs2)cc1]

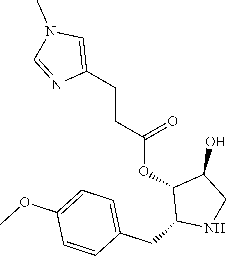 [SMILES: COc1ccc(C[C@H]2NC[C@H](O)[C@H]2OC(=O)CCc2cn(C)cn2)cc1]

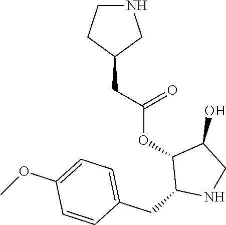 [SMILES: COc1ccc(C[C@H]2NC[C@H](O)[C@H]2OC(=O)C[C@H]2CCNC2)cc1]

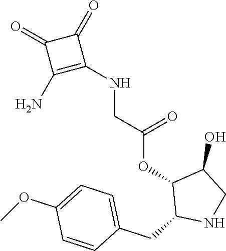 [SMILES: COc1ccc(C[C@H]2NC[C@H](O)[C@H]2OC(=O)CNc2c(N)c(=O)c2=O)cc1]

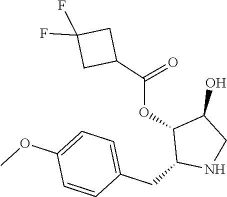 [SMILES: COc1ccc(C[C@H]2NC[C@H](O)[C@H]2OC(=O)C2CC(F)(F)C2)cc1]

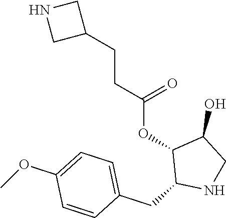 [SMILES: COc1ccc(C[C@H]2NC[C@H](O)[C@H]2OC(=O)CCC2CNC2)cc1]

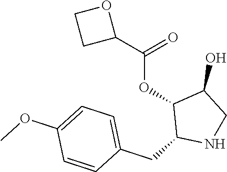 [SMILES: COc1ccc(C[C@H]2NC[C@H](O)[C@H]2OC(=O)C2CCO2)cc1]

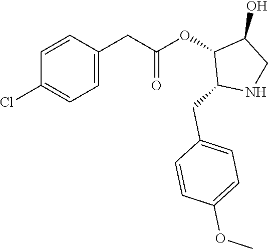 [SMILES: COc1ccc(C[C@H]2NC[C@H](O)[C@H]2OC(=O)Cc2ccc(Cl)cc2)cc1]

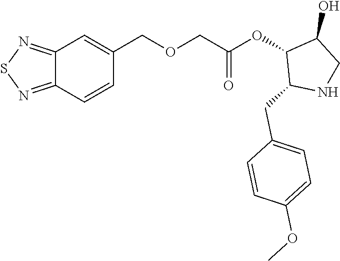 [SMILES: COc1ccc(C[C@H]2NC[C@H](O)[C@H]2OC(=O)COCc2ccc3nsnc3c2)cc1]

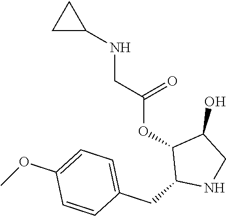 [SMILES: COc1ccc(C[C@H]2NC[C@H](O)[C@H]2OC(=O)CNC2CC2)cc1]

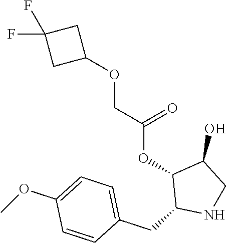 [SMILES: COc1ccc(C[C@H]2NC[C@H](O)[C@H]2OC(=O)COC2CC(F)(F)C2)cc1]

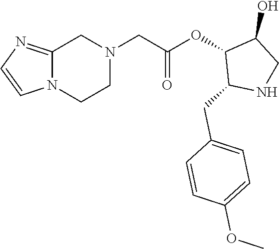 [SMILES: COc1ccc(C[C@H]2NC[C@H](O)[C@H]2OC(=O)CN2CCn3ccnc3C2)cc1]

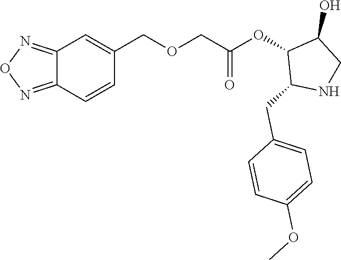 [SMILES: COc1ccc(C[C@H]2NC[C@H](O)[C@H]2OC(=O)COCc2ccc3nonc3c2)cc1]

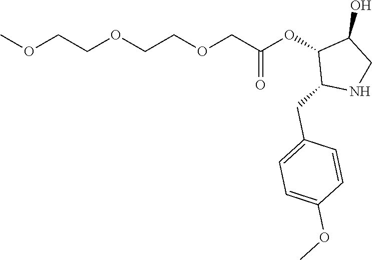 [SMILES: COCCOCCOCC(=O)O[C@@H]1[C@@H](O)CN[C@@H]1Cc1ccc(OC)cc1]

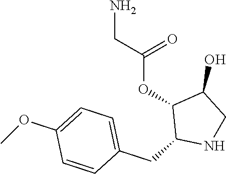 [SMILES: COc1ccc(C[C@H]2NC[C@H](O)[C@H]2OC(=O)CN)cc1]

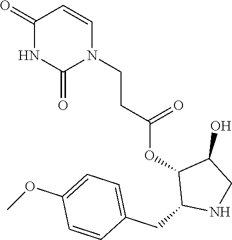 [SMILES: COc1ccc(C[C@H]2NC[C@H](O)[C@H]2OC(=O)CCn2ccc(=O)[nH]c2=O)cc1]

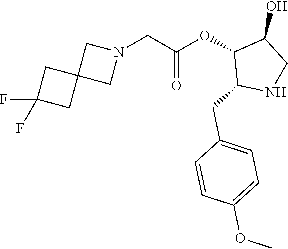 [SMILES: COc1ccc(C[C@H]2NC[C@H](O)[C@H]2OC(=O)CN2CC3(C2)CC(F)(F)C3)cc1]

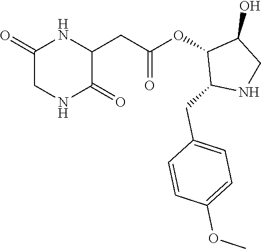 [SMILES: COc1ccc(C[C@H]2NC[C@H](O)[C@H]2OC(=O)CC2NC(=O)CNC2=O)cc1]

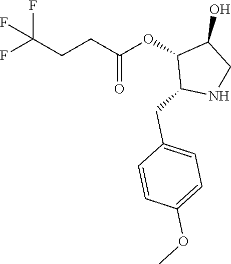 [SMILES: COc1ccc(C[C@H]2NC[C@H](O)[C@H]2OC(=O)CCC(F)(F)F)cc1]

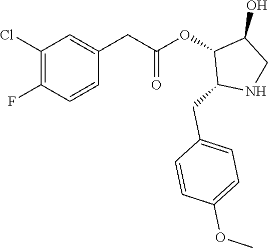 [SMILES: COc1ccc(C[C@H]2NC[C@H](O)[C@H]2OC(=O)Cc2ccc(F)c(Cl)c2)cc1]

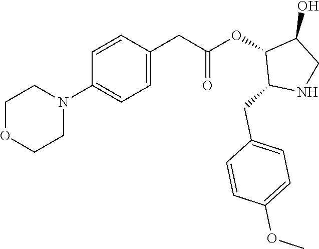 [SMILES: COc1ccc(C[C@H]2NC[C@H](O)[C@H]2OC(=O)Cc2ccc(N3CCOCC3)cc2)cc1]

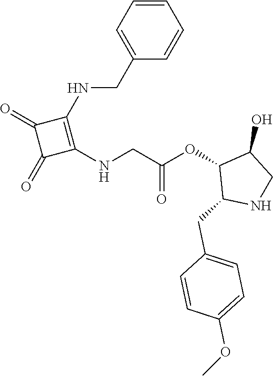 [SMILES: COc1ccc(C[C@H]2NC[C@H](O)[C@H]2OC(=O)CNc2c(NCc3ccccc3)c(=O)c2=O)cc1]

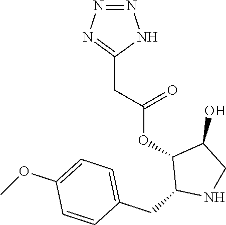 [SMILES: COc1ccc(C[C@H]2NC[C@H](O)[C@H]2OC(=O)Cc2nnn[nH]2)cc1]

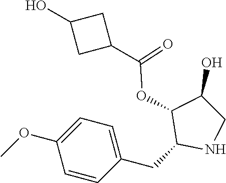 [SMILES: COc1ccc(C[C@H]2NC[C@H](O)[C@H]2OC(=O)C2CC(O)C2)cc1]

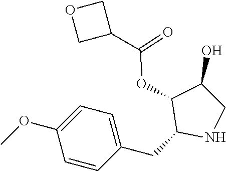 [SMILES: COc1ccc(C[C@H]2NC[C@H](O)[C@H]2OC(=O)C2COC2)cc1]

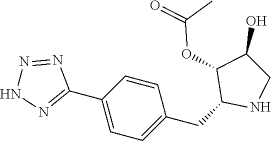 [SMILES: CC(=O)O[C@@H]1[C@@H](O)CN[C@@H]1Cc1ccc(-c2nn[nH]n2)cc1]

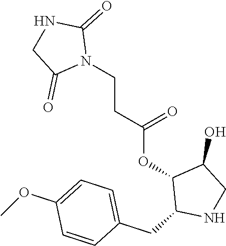 [SMILES: COc1ccc(C[C@H]2NC[C@H](O)[C@H]2OC(=O)CCN2C(=O)CNC2=O)cc1]

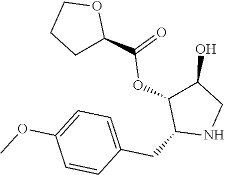 [SMILES: COc1ccc(C[C@H]2NC[C@H](O)[C@H]2OC(=O)[C@H]2CCCO2)cc1]

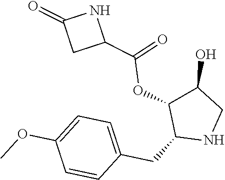 [SMILES: COc1ccc(C[C@H]2NC[C@H](O)[C@H]2OC(=O)C2CC(=O)N2)cc1]

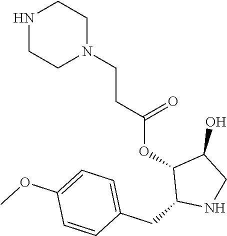 [SMILES: COc1ccc(C[C@H]2NC[C@H](O)[C@H]2OC(=O)CCN2CCNCC2)cc1]

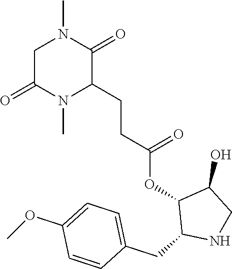 [SMILES: COc1ccc(C[C@H]2NC[C@H](O)[C@H]2OC(=O)CCC2C(=O)N(C)CC(=O)N2C)cc1]

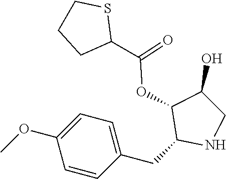 [SMILES: COc1ccc(C[C@H]2NC[C@H](O)[C@H]2OC(=O)C2CCCS2)cc1]

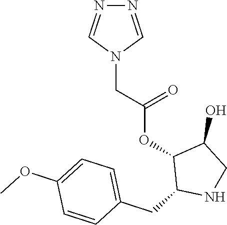 [SMILES: COc1ccc(C[C@H]2NC[C@H](O)[C@H]2OC(=O)Cn2cnnc2)cc1]

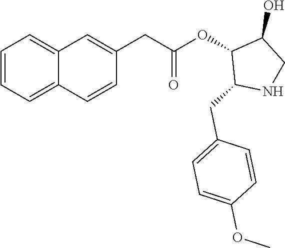 [SMILES: COc1ccc(C[C@H]2NC[C@H](O)[C@H]2OC(=O)Cc2ccc3ccccc3c2)cc1]

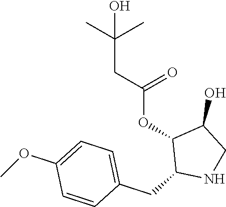 [SMILES: COc1ccc(C[C@H]2NC[C@H](O)[C@H]2OC(=O)CC(C)(C)O)cc1]

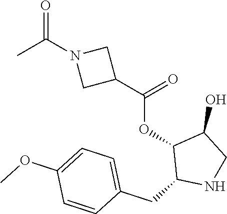 [SMILES: COc1ccc(C[C@H]2NC[C@H](O)[C@H]2OC(=O)C2CN(C(C)=O)C2)cc1]

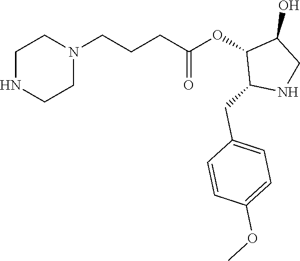 [SMILES: COc1ccc(C[C@H]2NC[C@H](O)[C@H]2OC(=O)CCCN2CCNCC2)cc1]

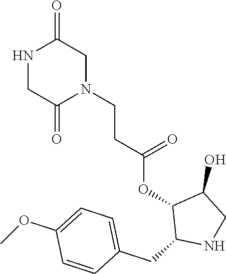 [SMILES: COc1ccc(C[C@H]2NC[C@H](O)[C@H]2OC(=O)CCN2CC(=O)NCC2=O)cc1]

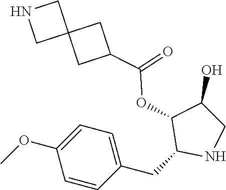 [SMILES: COc1ccc(C[C@H]2NC[C@H](O)[C@H]2OC(=O)C2CC3(CNC3)C2)cc1]

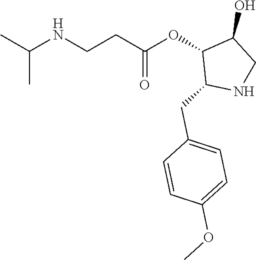 [SMILES: COc1ccc(C[C@H]2NC[C@H](O)[C@H]2OC(=O)CCNC(C)C)cc1]

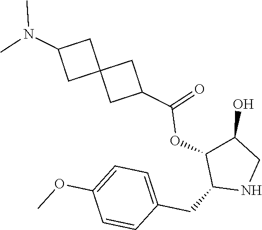 [SMILES: COc1ccc(C[C@H]2NC[C@H](O)[C@H]2OC(=O)C2CC3(C2)CC(N(C)C)C3)cc1]

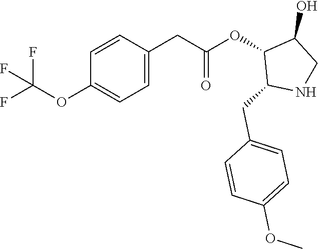 [SMILES: COc1ccc(C[C@H]2NC[C@H](O)[C@H]2OC(=O)Cc2ccc(OC(F)(F)F)cc2)cc1]

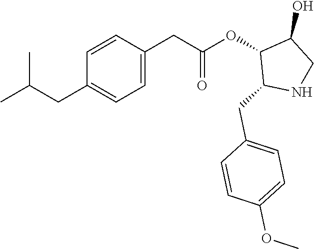 [SMILES: COc1ccc(C[C@H]2NC[C@H](O)[C@H]2OC(=O)Cc2ccc(CC(C)C)cc2)cc1]

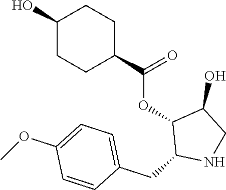 [SMILES: COc1ccc(C[C@H]2NC[C@H](O)[C@H]2OC(=O)[C@H]2CC[C@@H](O)CC2)cc1]

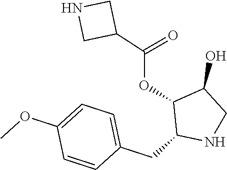 [SMILES: COc1ccc(C[C@H]2NC[C@H](O)[C@H]2OC(=O)C2CNC2)cc1]

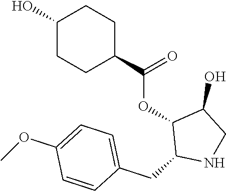 [SMILES: COc1ccc(C[C@H]2NC[C@H](O)[C@H]2OC(=O)[C@H]2CC[C@H](O)CC2)cc1]

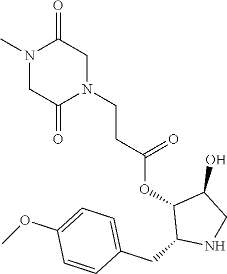 [SMILES: COc1ccc(C[C@H]2NC[C@H](O)[C@H]2OC(=O)CCN2CC(=O)N(C)CC2=O)cc1]

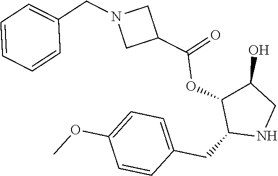 [SMILES: COc1ccc(C[C@H]2NC[C@H](O)[C@H]2OC(=O)C2CN(Cc3ccccc3)C2)cc1]

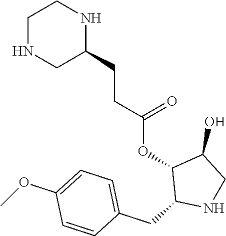 [SMILES: COc1ccc(C[C@H]2NC[C@H](O)[C@H]2OC(=O)CC[C@H]2CNCCN2)cc1]

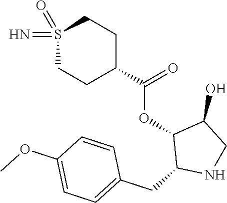 [SMILES: COc1ccc(C[C@H]2NC[C@H](O)[C@H]2OC(=O)[C@H]2CC[S@@](=N)(=O)CC2)cc1]

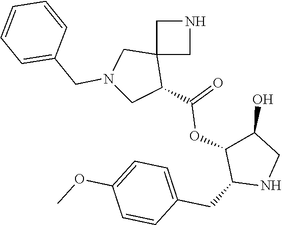 [SMILES: COc1ccc(C[C@H]2NC[C@H](O)[C@H]2OC(=O)[C@@H]2CN(Cc3ccccc3)CC23CNC3)cc1]

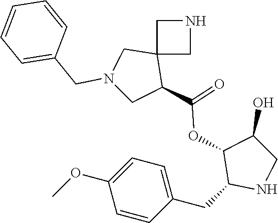 [SMILES: COc1ccc(C[C@H]2NC[C@H](O)[C@H]2OC(=O)[C@H]2CN(Cc3ccccc3)CC23CNC3)cc1]

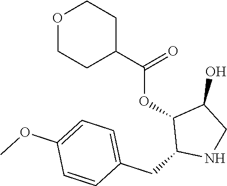 [SMILES: COc1ccc(C[C@H]2NC[C@H](O)[C@H]2OC(=O)C2CCOCC2)cc1]

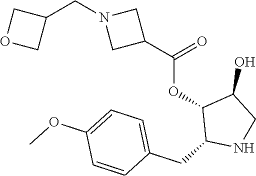 [SMILES: COc1ccc(C[C@H]2NC[C@H](O)[C@H]2OC(=O)C2CN(CC3COC3)C2)cc1]

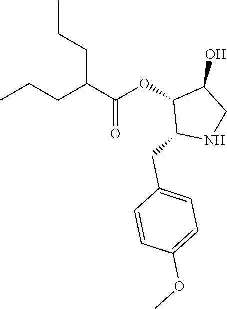 [SMILES: CCCC(CCC)C(=O)O[C@@H]1[C@@H](O)CN[C@@H]1Cc1ccc(OC)cc1]

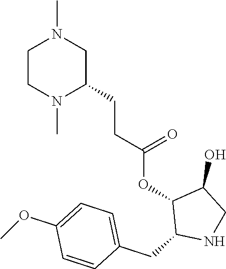 [SMILES: COc1ccc(C[C@H]2NC[C@H](O)[C@H]2OC(=O)CC[C@H]2CN(C)CCN2C)cc1]

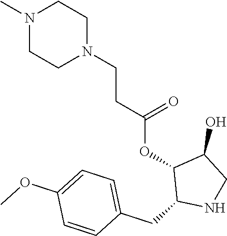 [SMILES: COc1ccc(C[C@H]2NC[C@H](O)[C@H]2OC(=O)CCN2CCN(C)CC2)cc1]

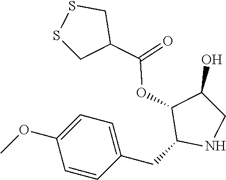 [SMILES: COc1ccc(C[C@H]2NC[C@H](O)[C@H]2OC(=O)C2CSSC2)cc1]

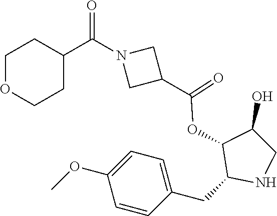 [SMILES: COc1ccc(C[C@H]2NC[C@H](O)[C@H]2OC(=O)C2CN(C(=O)C3CCOCC3)C2)cc1]

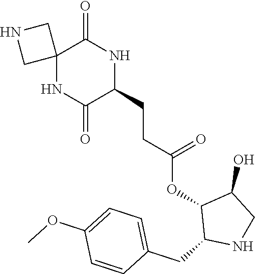 [SMILES: COc1ccc(C[C@H]2NC[C@H](O)[C@H]2OC(=O)CC[C@@H]2NC(=O)C3(CNC3)NC2=O)cc1]

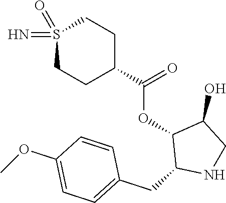 [SMILES: COc1ccc(C[C@H]2NC[C@H](O)[C@H]2OC(=O)[C@H]2CC[S@](=N)(=O)CC2)cc1]

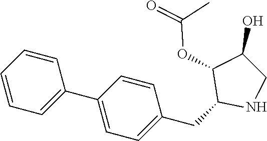 [SMILES: CC(=O)O[C@@H]1[C@@H](O)CN[C@@H]1Cc1ccc(-c2ccccc2)cc1]

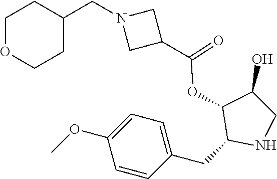 [SMILES: COc1ccc(C[C@H]2NC[C@H](O)[C@H]2OC(=O)C2CN(CC3CCOCC3)C2)cc1]

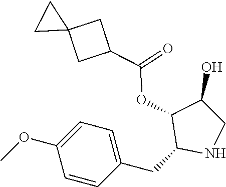 [SMILES: COc1ccc(C[C@H]2NC[C@H](O)[C@H]2OC(=O)C2CC3(CC3)C2)cc1]

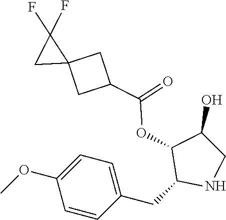 [SMILES: COc1ccc(C[C@H]2NC[C@H](O)[C@H]2OC(=O)C2CC3(C2)CC3(F)F)cc1]

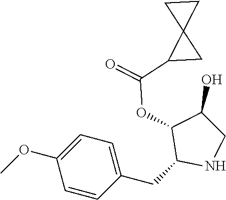 [SMILES: COc1ccc(C[C@H]2NC[C@H](O)[C@H]2OC(=O)C2CC23CC3)cc1]

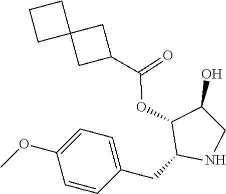 [SMILES: COc1ccc(C[C@H]2NC[C@H](O)[C@H]2OC(=O)C2CC3(CCC3)C2)cc1]

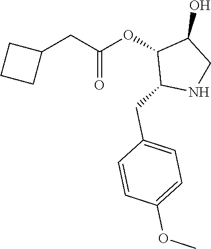 [SMILES: COc1ccc(C[C@H]2NC[C@H](O)[C@H]2OC(=O)CC2CCC2)cc1]

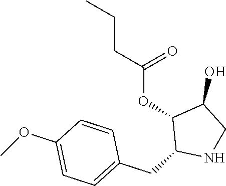 [SMILES: CCCC(=O)O[C@@H]1[C@@H](O)CN[C@@H]1Cc1ccc(OC)cc1]

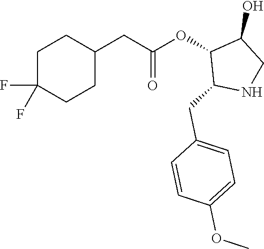 [SMILES: COc1ccc(C[C@H]2NC[C@H](O)[C@H]2OC(=O)CC2CCC(F)(F)CC2)cc1]

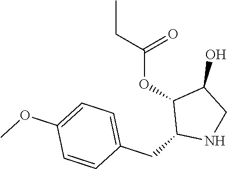 [SMILES: CCC(=O)O[C@@H]1[C@@H](O)CN[C@@H]1Cc1ccc(OC)cc1]

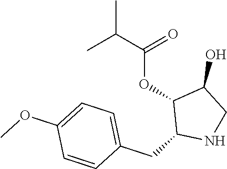 [SMILES: COc1ccc(C[C@H]2NC[C@H](O)[C@H]2OC(=O)C(C)C)cc1]

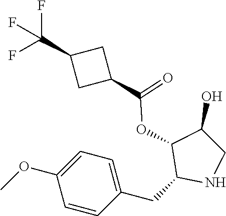 [SMILES: COc1ccc(C[C@H]2NC[C@H](O)[C@H]2OC(=O)[C@H]2C[C@@H](C(F)(F)F)C2)cc1]

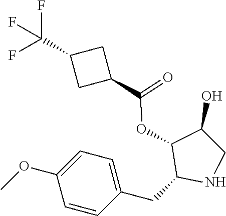 [SMILES: COc1ccc(C[C@H]2NC[C@H](O)[C@H]2OC(=O)[C@H]2C[C@H](C(F)(F)F)C2)cc1]

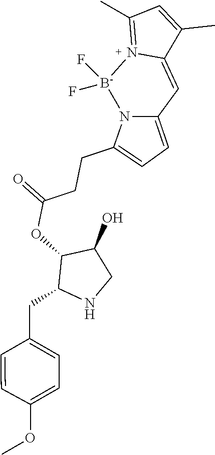

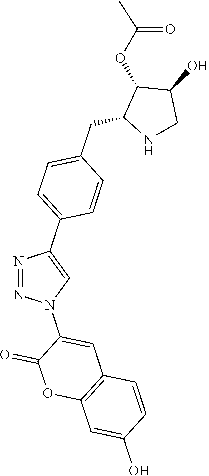 [SMILES: CC(=O)O[C@@H]1[C@@H](O)CN[C@@H]1Cc1ccc(-c2cn(-c3cc4ccc(O)cc4oc3=O)nn2)cc1]

In some embodiments, the compound of Formula (I) is selected from those in Table 1B, and pharmaceutically acceptable salts thereof.

TABLE 1B
Additional Compounds of Formula (I)

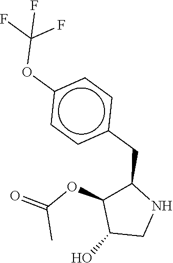

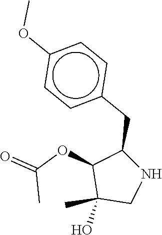

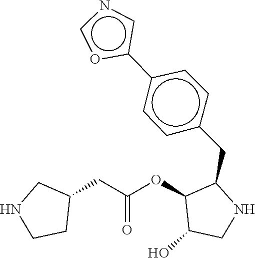

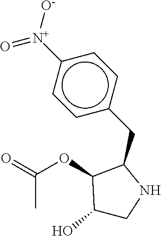

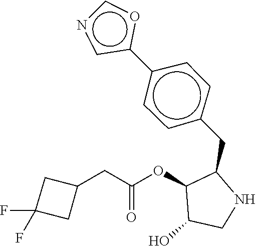

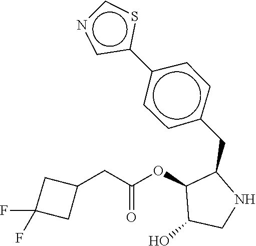

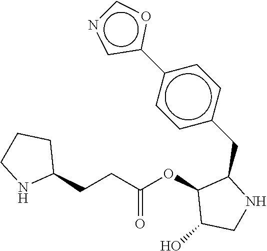

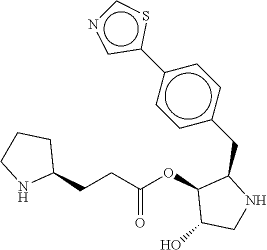

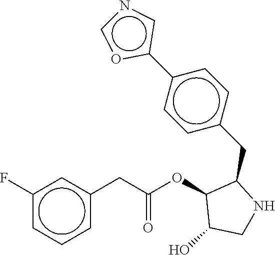

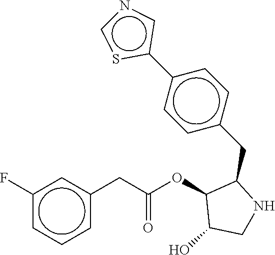

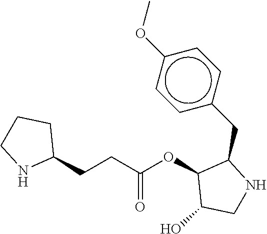

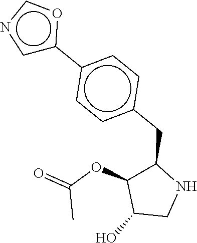

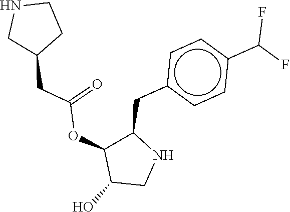

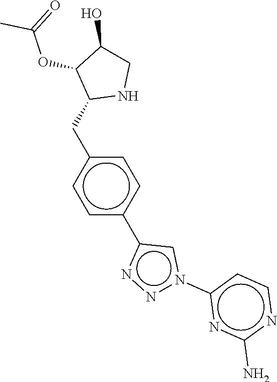

In some embodiments, the compound of Formula (I) is selected from those in Table 1A and Table 1B, and pharmaceutically acceptable salts thereof.

In some embodiments, a provided compound (a compound described herein, a compound of the present disclosure) is a compound of any of the formulae herein (e.g., Formula (I)) or pharmaceutically acceptable salt thereof. In some embodiments, a provided compound is a compound of any of the formulae herein (e.g., Formula (I)), or a salt thereof. In some embodiments, a provided compound is a compound of any of the formulae herein (e.g., Formula (I)).

### Pharmaceutical Compositions, Kits, and Administration

The present disclosure provides pharmaceutical compositions comprising a compound provided herein, or a pharmaceutically acceptable salt thereof (e.g., a compound of Formula (I), or a pharmaceutically acceptable salt thereof), and optionally a pharmaceutically acceptable excipient. In some embodiments, the pharmaceutical composition described herein comprises a compound provided herein, or a pharmaceutically acceptable salt thereof, and a pharmaceutically acceptable excipient. In certain embodiments, a compound described herein is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable carrier or excipient, and/or any additional ingredients in a pharmaceutical composition described herein will vary, depending upon the identity, size, and/or condition of the subject treated and further depending upon the route by which the composition is to be administered.

Pharmaceutically acceptable carriers/excipients used in the manufacture of provided pharmaceutical compositions include inert diluents, solvents, dispersing and/or granulating agents, surface active agents and/or emulsifiers, disintegrating agents, binding agents, preservatives, buffering agents, lubricating agents, oils, butters, and/or waxes. Excipients such as coloring agents, coating agents, sweetening agents, flavoring agents, and fragrances may also be present in the composition.

The compounds and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form a single unit dosage form. Thus, in one aspect, provided are kits including a first container comprising a compound or pharmaceutical composition described herein. In certain embodiments, the kits are useful for treating and/or preventing a disease, disorder, or condition in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease in a subject in need thereof. A kit described herein may include one or more additional pharmaceutical agents described herein as a separate composition.

### Methods of Modulating Protein Synthesis

In another aspect, the present disclosure provides a method of modulating protein synthesis in a subject in need thereof or in a cell, tissue, or biological sample, comprising administering to the subject in need thereof or contacting the cell, tissue, or biological sample with an effective amount of a provided compound (e.g., a compound of the present disclosure, or a pharmaceutically acceptable salt thereof), or a pharmaceutical composition thereof. In another aspect, the present disclosure provides a method of modulating protein synthesis in a subject in need thereof or in a cell, tissue, or biological sample, comprising administering to the subject in need thereof or contacting the cell, tissue, or biological sample with an effective amount of a compound of Formula (I), or a pharmaceutical composition thereof.

In some embodiments, the present disclosure provides a method of modulating protein synthesis in a subject in need thereof, comprising administering to the subject in need thereof an effective amount of a provided compound, or a pharmaceutical composition thereof. In some embodiments, the present disclosure provides a method of modulating protein synthesis in a cell, tissue, or biological sample, comprising contacting the cell, tissue, or biological sample with an effective amount of a provided compound, or a pharmaceutical composition thereof. In some embodiments, the present disclosure provides a provided compound, or a pharmaceutical composition thereof, for use in modulating protein synthesis in a subject in need thereof. In some embodiments, the present disclosure provides a provided compound, or a pharmaceutical composition thereof, for use in the manufacture of a medicament for modulating protein synthesis in a subject in need thereof.

In some embodiments modulating protein synthesis comprises modulating synthesis of a target protein. In some embodiments, modulating protein synthesis comprises decreasing protein synthesis. In some embodiments, the protein synthesis is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, protein synthesis is decreased by not more than about 10%, not more than about 20%, not more than about 30%, not more than about 40%, not more than about 50%, not more than about 60%, not more than about 70%, not more than about 80%, not more than about 90%, not more than about 95%, or not more than about 98%. In some embodiments, protein synthesis is decreased by a range between a percentage described in this paragraph and another percentage described in this paragraph, inclusive.

In another aspect, the present disclosure provides a method of decreasing protein synthesis in a subject in need thereof or in a cell, tissue, or biological sample, comprising administering to the subject in need thereof or contacting the cell, tissue, or biological sample with an effective amount of a provided compound (e.g., a compound of the present disclosure, or a pharmaceutically acceptable salt thereof), or a pharmaceutical composition thereof. In some embodiments, the present disclosure provides a method of decreasing protein synthesis in a subject in need thereof, comprising administering to the subject in need thereof an effective amount of a provided compound, or a pharmaceutical composition thereof. In some embodiments, the present disclosure provides a method of decreasing protein synthesis in a cell, tissue, or biological sample, comprising contacting the cell, tissue, or biological sample with an effective amount of a provided compound, or a pharmaceutical composition thereof. In some embodiments, the present disclosure provides a provided compound, or a pharmaceutical composition thereof, for use in decreasing protein synthesis in a subject in need thereof. In some embodiments, the present disclosure provides a provided compound, or a pharmaceutical composition thereof, for use in the manufacture of a medicament for decreasing protein synthesis in a subject in need thereof.

In some embodiments, decreasing protein synthesis comprises decreasing synthesis of a target protein. In some embodiments, the protein synthesis is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, protein synthesis is decreased by not more than about 10%, not more than about 20%, not more than about 30%, not more than about 40%, not more than about 50%, not more than about 60%, not more than about 70%, not more than about 80%, not more than about 90%, not more than about 95%, or not more than about 98%. In some embodiments, protein synthesis is decreased by a range between a percentage described in this paragraph and another percentage described in this paragraph, inclusive.

In some embodiments, the method is selective for decreasing synthesis of a first protein compared to synthesis of a second protein. In some embodiments, the ratio of the decrease in synthesis of the first protein to the decrease in synthesis of the second protein is about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 6:1, about 7:1, about 8:1, about 9:1, about 10:1, about 15:1, about 20:1, about 25:1, about 50:1, about 75:1, about 100:1, about 200:1, about 300:1, about 400:1, about 500:1, about 1,000:1, about 10,000:1, or about 100,000:1. In some embodiments, the ratio of the decrease in synthesis of the first protein to the decrease in synthesis of the second protein is between a ratio described in this paragraph and another ratio described in this paragraph, inclusive.

In some embodiments, the method further comprises decreasing an amount of mRNA, wherein the mRNA is associated with synthesis of the target protein. In some embodiments, the amount of mRNA is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, the amount of mRNA is decreased by not more than 10%, not more than 20%, not more than 30%, not more than 40%, not more than 50%, not more than 60%, not more than 70%, not more than 80%, not more than 90%, not more than 95%, or not more than 98%. In some embodiments, the amount of mRNA is decreased by a range between a percentage described in this paragraph and another percentage described in this paragraph, inclusive.

In some embodiments, the method is in vitro. In some embodiments, the method is in vivo.

In some embodiments, the target protein is B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D). In some embodiments, the target protein is BCL-2. In some embodiments, the target protein is MYC. In some embodiments, the target protein is CCND1. In some embodiments, the target protein is MCL-1. In some embodiments, the target protein is ALK. The target proteins KRAS-G12D. BCL-2, MYC, CCND1, MCL-1, ALK, and KRAS-G12D are exemplary target proteins, and the methods disclosed herein are not limited to these target proteins.

In some embodiments, the cell is a cancer cell. In some embodiments, the cell is a HEK 293T, HPAF-II, KLE, LS411N, MCF7, NCI-H1915, HCC38, HEPG2, KATO-III, MS751, or T47D cell. In some embodiments, the cell is a HEK 293T cell. In some embodiments, the cell is a HPAF-II cell. In some embodiments, the cell is a KLE cell. In some embodiments, the cell is a LS411N cell. In some embodiments, the cell is a MCF7 cell. In some embodiments, the cell is a NCI-H1915 cell. In some embodiments, the cell is a HCC38 cell. In some embodiments, the cell is a HEPG2 cell. In some embodiments, the cell is a KATO-III cell. In some embodiments, the cell is a MS751 cell. In some embodiments, the cell is a T47D cell.

### Methods of Treatment and Prevention

In another aspect, the present disclosure provides a method of treating or preventing a disease in a subject in need thereof, comprising administering to the subject in need thereof a therapeutically effective amount of a provided compound (e.g., a compound of the present disclosure, or a pharmaceutically acceptable salt thereof), or a pharmaceutical composition thereof. In some embodiments, the present disclosure provides a method of treating a disease in a subject in need thereof, comprising administering to the subject in need thereof a therapeutically effective amount of a provided compound, or a pharmaceutical composition thereof. In some embodiments, the present disclosure provides a method of preventing a disease in a subject in need thereof, comprising administering to the subject in need thereof a therapeutically effective amount of a provided compound, or a pharmaceutical composition thereof. In another aspect, the present disclosure provides a method of treating or preventing a disease in a subject in need thereof, comprising administering to the subject in need thereof a therapeutically effective amount of a compound of Formula (I), or a pharmaceutical composition thereof.

In some embodiments, the present disclosure provides a provided compound, or a pharmaceutical composition thereof, for use in treating or preventing a disease in a subject in need thereof. In some embodiments, the present disclosure provides a provided compound, or a pharmaceutical composition thereof, for use in treating a disease in a subject in need thereof. In some embodiments, the present disclosure provides a provided compound, or a pharmaceutical composition thereof, for use in preventing a disease in a subject in need thereof.

In some embodiments, the present disclosure provides a provided compound, or a pharmaceutical composition thereof, for use in the manufacture of a medicament for treating or preventing a disease in a subject in need thereof. In some embodiments, the present disclosure provides a provided compound, or a pharmaceutical composition thereof, for use in the manufacture of a medicament for treating a disease in a subject in need thereof. In some embodiments, the present disclosure provides a provided compound, or a pharmaceutical composition thereof, for use in the manufacture of a medicament for preventing a disease in a subject in need thereof.

In some embodiments, the disease is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

In some embodiments, the disease is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)). In some embodiments, the proliferative disease is cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma). In some embodiments, the cancer is prostate cancer (e.g., prostate adenocarcinoma), pancreatic cancer (e.g., pancreatic andenocarcinoma, intraductal papillary mucinous neoplasm (IPMN), Islet cell tumors), lung cancer (e.g., bronchogenic carcinoma, small cell lung cancer (SCLC), non-small cell lung cancer (NSCLC), adenocarcinoma of the lung), breast cancer (e.g., adenocarcinoma of the breast, papillary carcinoma of the breast, mammary cancer, medullary carcinoma of the breast, triple-negative breast cancer (TNBC)), colorectal cancer (e.g., colon cancer, rectal cancer, colorectal adenocarcinoma), endometrial cancer (e.g., uterine cancer, uterine sarcoma), ovarian cancer (e.g., cystadenocarcinoma, ovarian embryonal carcinoma, ovarian adenocarcinoma), cervical cancer (e.g., cervical adenocarcinoma), esophageal cancer (e.g., adenocarcinoma of the esophagus, Barrett's adenocarcinoma), bladder cancer, biliary cancer (e.g., cholangiocarcinoma), hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), or neuroblastoma.

In some embodiments, the cancer is prostate cancer (e.g., prostate adenocarcinoma). In some embodiments, the cancer is prostate adenocarcinoma. In some embodiments, the cancer is pancreatic cancer (e.g., pancreatic andenocarcinoma, intraductal papillary mucinous neoplasm (IPMN), Islet cell tumors). In some embodiments, the cancer is pancreatic andenocarcinoma. In some embodiments, the cancer is intraductal papillary mucinous neoplasm (IPMN). In some embodiments, the cancer is Islet cell tumors. In some embodiments, the cancer is lung cancer (e.g., bronchogenic carcinoma, small cell lung cancer (SCLC), non-small cell lung cancer (NSCLC), adenocarcinoma of the lung). In some embodiments, the cancer is bronchogenic carcinoma. In some embodiments, the cancer is small cell lung cancer (SCLC). In some embodiments, the cancer is non-small cell lung cancer (NSCLC). In some embodiments, the cancer is adenocarcinoma of the lung. In some embodiments, the cancer is breast cancer (e.g., adenocarcinoma of the breast, papillary carcinoma of the breast, mammary cancer, medullary carcinoma of the breast, triple-negative breast cancer (TNBC)). In some embodiments, the cancer is adenocarcinoma of the breast. In some embodiments, the cancer is papillary carcinoma of the breast. In some embodiments, the cancer is mammary cancer. In some embodiments, the cancer is medullary carcinoma of the breast. In some embodiments, the cancer is triple-negative breast cancer (TNBC). In some embodiments, the cancer is colorectal cancer (e.g., colon cancer, rectal cancer, colorectal adenocarcinoma). In some embodiments, the cancer is colon cancer. In some embodiments, the cancer is rectal cancer. In some embodiments, the cancer is colorectal adenocarcinoma. In some embodiments, the cancer is endometrial cancer (e.g., uterine cancer, uterine sarcoma). In some embodiments, the cancer is uterine cancer. In some embodiments, the cancer is uterine sarcoma. In some embodiments, the cancer is ovarian cancer (e.g., cystadenocarcinoma, ovarian embryonal carcinoma, ovarian adenocarcinoma). In some embodiments, the cancer is cystadenocarcinoma. In some embodiments, the cancer is ovarian embryonal carcinoma. In some embodiments, the cancer is ovarian adenocarcinoma. In some embodiments, the cancer is cervical cancer (e.g., cervical adenocarcinoma). In some embodiments, the cancer is cervical adenocarcinoma. In some embodiments, the cancer is esophageal cancer (e.g., adenocarcinoma of the esophagus, Barrett's adenocarcinoma). In some embodiments, the cancer is adenocarcinoma of the esophagus. In some embodiments, the cancer is Barrett's adenocarcinoma. In some embodiments, the cancer is bladder cancer. In some embodiments, the cancer is biliary cancer (e.g., cholangiocarcinoma). In some embodiments, the cancer is cholangiocarcinoma. In some embodiments, the cancer is hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)). In some embodiments, the cancer is leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)). In some embodiments, the cancer is acute lymphocytic leukemia (ALL) (e.g., B-cell ALL, T-cell ALL). In some embodiments, the cancer is B-cell ALL. In some embodiments, the cancer is T-cell ALL. In some embodiments, the cancer is acute myelocytic leukemia (AML) (e.g., B-cell AML, T-cell AML). In some embodiments, the cancer is B-cell AML. In some embodiments, the cancer is T-cell AML. In some embodiments, the cancer is chronic myelocytic leukemia (CML) (e.g., B-cell CML, T-cell CML). In some embodiments, the cancer is B-cell CML. In some embodiments, the cancer is T-cell CML. In some embodiments, the cancer is chronic lymphocytic leukemia (CLL) (e.g., B-cell CLL, T-cell CLL). In some embodiments, the cancer is B-cell CLL. In some embodiments, the cancer is T-cell CLL. In some embodiments, the cancer is lymphoma (e.g., Hodgkin lymphoma (HL) (e.g., B-cell HL, T-cell HL); non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma); T-cell NHL (e.g., precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma)). In some embodiments, the cancer is Hodgkin lymphoma (HL) (e.g., B-cell HL, T-cell HL). In some embodiments, the cancer is B-cell HL. In some embodiments, the cancer is T-cell HL. In some embodiments, the cancer is non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma). In some embodiments, the cancer is diffuse large cell lymphoma (DLCL) (e.g., diffuse large B-cell lymphoma). In some embodiments, the cancer is diffuse large B-cell lymphoma. In some embodiments, the cancer is follicular lymphoma. In some embodiments, the cancer is chronic lymphocytic leukemia/small lymphocytic lymphoma (CLL/SLL). In some embodiments, the cancer is mantle cell lymphoma (MCL). In some embodiments, the cancer is marginal zone B-cell lymphomas (e.g., mucosa-associated lymphoid tissue (MALT) lymphomas, nodal marginal zone B-cell lymphoma, splenic marginal zone B-cell lymphoma). In some embodiments, the cancer is mucosa-associated lymphoid tissue (MALT) lymphomas. In some embodiments, the cancer is nodal marginal zone B-cell lymphoma. In some embodiments, the cancer is splenic marginal zone B-cell lymphoma. In some embodiments, the cancer is primary mediastinal B-cell lymphoma. In some embodiments, the cancer is Burkitt lymphoma. In some embodiments, the cancer is lymphoplasmacytic lymphoma (i.e., Waldenström's macroglobulinemia). In some embodiments, the cancer is Waldenström's macroglobulinemia. In some embodiments, the cancer is hairy cell leukemia (HCL). In some embodiments, the cancer is immunoblastic large cell lymphoma. In some embodiments, the cancer is precursor B-lymphoblastic lymphoma. In some embodiments, the cancer is primary central nervous system (CNS) lymphoma. In some embodiments, the cancer is T-cell NHL (e.g., precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma)). In some embodiments, the cancer is precursor T-lymphoblastic lymphoma/leukemia. In some embodiments, the cancer is peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma). In some embodiments, the cancer is cutaneous T-cell lymphoma (CTCL) (e.g., mycosis fungoides, Sezary syndrome). In some embodiments, the cancer is mycosis fungoides. In some embodiments, the cancer is Sezary syndrome. In some embodiments, the cancer is angioimmunoblastic T-cell lymphoma. In some embodiments, the cancer is extranodal natural killer T-cell lymphoma. In some embodiments, the cancer is enteropathy type T-cell lymphoma. In some embodiments, the cancer is subcutaneous panniculitis-like T-cell lymphoma. In some embodiments, the cancer is anaplastic large cell lymphoma. In some embodiments, the cancer is a mixture of one or more leukemia/lymphoma as described above. In some embodiments, the cancer is multiple myeloma (MM). In some embodiments, the cancer is neuroblastoma.

In some embodiments, the disease is a neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)). In some embodiments, the neurological disease is cerebellar ataxia. In some embodiments, the neurological disease is a neurodegenerative disease (e.g., Alzheimer's disease, Parkinson's disease, amyotrophic lateral sclerosis, tauopathy (including frontotemporal dementia), Huntington's disease, Friedreich's ataxia). In some embodiments, the neurodegenerative disease is Alzheimer's disease. In some embodiments, the neurodegenerative disease is Parkinson's disease. In some embodiments, the neurodegenerative disease is amyotrophic lateral sclerosis. In some embodiments, the neurodegenerative disease is tauopathy (e.g., frontotemporal dementia). In some embodiments, the neurodegenerative disease is frontotemporal dementia. In some embodiments, the neurodegenerative disease is Huntington's disease. In some embodiments, the neurodegenerative disease is Friedreich's ataxia.

In some embodiments, the disease is an immune disorder (e.g., psoriasis, lupus, rheumatoid arthritis). In some embodiments, the immune disorder is psoriasis. In some embodiments, the immune disorder is lupus. In some embodiments, the immune disorder is rheumatoid arthritis.

In some embodiments, the disease is associated with BCL-2, MYC, CCND1, MCL-1, ALK, or KRAS-G12D. In some embodiments, the disease is associated with MYC, ALK, or KRAS-G12D.

In some embodiments, the disease associated with BCL-2, MYC, CCND1, MCL-1, ALK, or KRAS-G12D is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

In certain embodiments, the disease or disorder is cancer. In some embodiments, the cancer is breast cancer, lung cancer, prostate cancer, bladder cancer, liver cancer, colorectal cancer, endometrial cancer, ovarian cancer, pancreatic cancer, esophagus cancer, gastric cancer, esophageal cancer, uterine cancer, skin cancer, leukemia, or lymphoma.

In some embodiments, the disease or disorder is Breast cancer, NSCLC, Prostate cancer, Bladder cancer, Colorectal cancer, Endometrial cancer, Melanoma, Ovarian cancer, Pancreatic cancer, Hepatocellular cancer, Esophagus cancer, Gastric cancer, Diffuse Large B-cell lymphoma, Uterine sarcoma, or Acute myeloid leukemia.

In certain embodiments, the disease or disorder is Acral Lentiginous Melanoma, Acute Lymphoblastic Leukemia, Acute Myeloid Leukemia, Adenocarcinoma of the Gastroesophageal Junction, AL Amyloidosis, ALK-Positive Anaplastic Large Cell Lymphoma, ALK-Positive Large B-Cell Lymphoma, Anal Carcinoma, Anaplastic Large Cell Lymphoma, Astrocytoma, B-Cell Acute Lymphoblastic Leukemia, B-Cell Lymphoma, B-Cell Non-Hodgkin Lymphoma, Biliary Tract Carcinoma, Bladder Carcinoma, Bladder Papillary Urothelial Neoplasm, Brain Glioblastoma, Breast Angiosarcoma, Breast Carcinoma, Bronchogenic Carcinoma, Burkitt Lymphoma, Carcinoma, Central Nervous System Neoplasm, Cholangiocarcinoma, Chondrosarcoma, Chronic Lymphocytic Leukemia, Chronic Lymphocytic Leukemia/Small Lymphocytic Lymphoma, Chronic Myeloid Leukemia, Colon Carcinoma, Colorectal Adenocarcinoma, Colorectal Carcinoma, Dedifferentiated Chondrosarcoma, Desmoid-Type Fibromatosis, Desmoplastic/Nodular Medulloblastoma, Diffuse Large B-Cell Lymphoma, Diffuse Large B-Cell Lymphoma Activated B-Cell Type, Double-Hit Lymphoma, EBV-Positive Diffuse Large B-Cell Lymphoma, Endometrial Serous Adenocarcinoma, Erdheim-Chester Disease, Esophageal Adenocarcinoma, Esophageal Adenosquamous Carcinoma, Esophageal Carcinoma, Esophageal Squamous Cell Carcinoma, Ewing Sarcoma, Follicular Lymphoma, Ganglioneuroblastoma, Gastric Adenocarcinoma, Gastric Adenosquamous Carcinoma, Gastric Carcinoma, Gastric Squamous Cell Carcinoma, Germinal Center B-Cell-Like Diffuse Large B-Cell Lymphoma, Glioblastoma, Hairy Cell Leukemia, Head and Neck Carcinoma, Head and Neck Squamous Cell Carcinoma, Hematopoietic and Lymphoid Malignancy, Hepatocellular Carcinoma, High Grade B-Cell Lymphoma, High Grade B-Cell Lymphoma with MYC and BCL2 and/or BCL6 Rearrangements, High Grade Ovarian Serous Adenocarcinoma, Histiocytic and Dendritic Cell Neoplasm, Hodgkin Lymphoma, Hypopharyngeal Squamous Cell Carcinoma, Inflammatory Myofibroblastic Tumor, Intracranial Primitive Neuroectodermal Neoplasm, Intrahepatic Cholangiocarcinoma, Intraocular Lymphoma, Invasive Breast Carcinoma, Juvenile Myelomonocytic Leukemia, Langerhans Cell Histiocytosis, Large Cell/Anaplastic Medulloblastoma, Laryngeal Squamous Cell Carcinoma, Leukemia, Low Grade Glioma, Lung Adenocarcinoma, Lung Carcinoma, Lymphoma, Lymphoplasmacytic Lymphoma, Malignant Breast Neoplasm, Malignant Central Nervous System Neoplasm, Malignant Colon Neoplasm, Malignant Colorectal Neoplasm, Malignant Endometrial Neoplasm, Malignant Gastric Neoplasm, Malignant Glioma, Malignant Lung Neoplasm, Malignant Ovarian Epithelial Tumor, Malignant Ovarian Neoplasm, Malignant Pancreatic Neoplasm, Malignant Pleural Mesothelioma, Malignant Prostate Neoplasm, Malignant Solid Tumor, Malignant Thyroid Gland Neoplasm, Mantle Cell Lymphoma, Marginal Zone Lymphoma, Mature B-Cell Lymphoma/Leukemia, Mature B-Cell Non-Hodgkin Lymphoma, Mature T-Cell and NK-Cell Lymphoma/Leukemia, Medulloblastoma, Medulloblastoma with Extensive Nodularity, Melanoma, Merkel Cell Carcinoma, Multiple Myeloma, Myelodysplastic/Myeloproliferative Neoplasm, Myeloid Neoplasm, Nasopharyngeal Carcinoma, Neuroblastoma, Non-Hodgkin Lymphoma, Non-Small Cell Lung Carcinoma, Non-Squamous Non-Small Cell Lung Carcinoma, Oral Cavity Carcinoma, Oropharyngeal Squamous Cell Carcinoma, Osteosarcoma, Ovarian Carcinoma, Pancreatic Adenocarcinoma, Pancreatic Carcinoma, Pancreatic Ductal Adenocarcinoma, Penile Carcinoma, Peripheral T-Cell Lymphoma, Primary Central Nervous System Lymphoma, Primary Cutaneous Anaplastic Large Cell Lymphoma, Primary Malignant Liver Neoplasm, Prostate Carcinoma, Rosai-Dorfman Disease, Sarcoma, Small Cell Lung Carcinoma, Small Intestinal Carcinoma, Small Intestinal Lymphoma, Small Lymphocytic Leukemia, Small Lymphocytic Lymphoma, Soft Tissue Sarcoma, Squamous Cell Lung Carcinoma, Synovial Sarcoma, Systemic Anaplastic Large Cell Lymphoma, Thyroid Gland Follicular Carcinoma, Thyroid Gland Medullary Carcinoma, Thyroid Gland Undifferentiated (Anaplastic) Carcinoma, Transformed Non-Hodgkin Lymphoma, Triple-Hit Lymphoma, Urothelial Carcinoma, Vaginal Carcinoma, or Vulvar Carcinoma.

In some embodiments, the disease or disorder is associated with KRAS-G12D. In some embodiments, the disease or disorder is mediated by KRAS-G12D. In some embodiments, the disease or disorder is Juvenile Myelomonocytic Leukemia, Non-Small Cell Lung Carcinoma, Pancreatic Adenocarcinoma, Malignant Ovarian Neoplasm, Colorectal Carcinoma, Malignant Endometrial Neoplasm, Cholangiocarcinoma, Malignant Solid Tumor, Malignant Ovarian Epithelial Tumor, Esophageal Adenocarcinoma, Biliary Tract Carcinoma, Carcinoma, Colorectal Adenocarcinoma, Malignant Gastric Neoplasm, Malignant Colon Neoplasm, Neuroblastoma, Malignant Colorectal Neoplasm, Pancreatic Carcinoma, Intrahepatic Cholangiocarcinoma, Melanoma, Malignant Lung Neoplasm, Myelodysplastic/Myeloproliferative Neoplasm, Pancreatic Ductal Adenocarcinoma, Thyroid Gland Follicular Carcinoma, Malignant Pancreatic Neoplasm, or Lung Adenocarcinoma.

In some embodiments, the disease or disorder is associated with ALK. In some embodiments, the disease or disorder is mediated by ALK. In certain embodiments, the disease or disorder is Small Intestinal Carcinoma, Non-Small Cell Lung Carcinoma, Colon Carcinoma, Soft Tissue Sarcoma, Small Cell Lung Carcinoma, Astrocytoma, Rosai-Dorfman Disease, Colorectal Carcinoma, Malignant Thyroid Gland Neoplasm, Cholangiocarcinoma, Non-Squamous Non-Small Cell Lung Carcinoma, Glioblastoma, Malignant Solid Tumor, Acute Myeloid Leukemia, Leukemia, Erdheim-Chester Disease, Malignant Glioma, Hepatocellular Carcinoma, ALK-Positive Large B-Cell Lymphoma, B-Cell Non-Hodgkin Lymphoma, Thyroid Gland Medullary Carcinoma, EBV-Positive Diffuse Large B-Cell Lymphoma, Colorectal Adenocarcinoma, Systemic Anaplastic Large Cell Lymphoma, Thyroid Gland Undifferentiated (Anaplastic) Carcinoma, Malignant Colon Neoplasm, Hematopoietic and Lymphoid Malignancy, ALK-Positive Anaplastic Large Cell Lymphoma, Diffuse Large B-Cell Lymphoma, Malignant Pleural Mesothelioma, Squamous Cell Lung Carcinoma, Non-Hodgkin Lymphoma, Neuroblastoma, Ganglioneuroblastoma, Malignant Central Nervous System Neoplasm, Primary Cutaneous Anaplastic Large Cell Lymphoma, Malignant Colorectal Neoplasm, Low Grade Glioma, Gastric Carcinoma, Multiple Myeloma, Inflammatory Myofibroblastic Tumor, Pancreatic Carcinoma, Melanoma, Malignant Breast Neoplasm, Malignant Lung Neoplasm, Anaplastic Large Cell Lymphoma, Histiocytic and Dendritic Cell Neoplasm, Central Nervous System Neoplasm, Mature T-Cell and NK-Cell Lymphoma/Leukemia, Esophageal Carcinoma, Pancreatic Ductal Adenocarcinoma, Adenocarcinoma of the Gastroesophageal Junction, Langerhans Cell Histiocytosis, Lymphoma, Mature B-Cell Lymphoma/Leukemia, or Lung Adenocarcinoma.

In certain embodiments, the disease or disorder is associated with CCND-1. In certain embodiments, the disease or disorder is mediated by CCND-1. In some embodiments, the disease or disorder is Non-Small Cell Lung Carcinoma, Malignant Ovarian Neoplasm, Soft Tissue Sarcoma, Malignant Prostate Neoplasm, Oropharyngeal Squamous Cell Carcinoma, Nasopharyngeal Carcinoma, Mature B-Cell Lymphoma/Leukemia, Lung Carcinoma, Oral Cavity Carcinoma, Primary Central Nervous System Lymphoma, Laryngeal Squamous Cell Carcinoma, Malignant Solid Tumor, Osteosarcoma, Bronchogenic Carcinoma, AL Amyloidosis, Bladder Carcinoma, Malignant Glioma, B-Cell Non-Hodgkin Lymphoma, Hypopharyngeal Squamous Cell Carcinoma, Mantle Cell Lymphoma, Squamous Cell Lung Carcinoma, Non-Hodgkin Lymphoma, Malignant Colorectal Neoplasm, Multiple Myeloma, Melanoma, Malignant Breast Neoplasm, Acral Lentiginous Melanoma, Breast Carcinoma, Dedifferentiated Chondrosarcoma, Anaplastic Large Cell Lymphoma, Head and Neck Squamous Cell Carcinoma, Histiocytic and Dendritic Cell Neoplasm, Bladder Papillary Urothelial Neoplasm, Primary Malignant Liver Neoplasm, Chondrosarcoma, Lymphoma, Malignant Pancreatic Neoplasm, or Urothelial Carcinoma.

In some embodiments, the disease or disorder is associated with CCNE1. In some embodiments, the disease or disorder is mediated by CCNE1. In certain embodiments, the disease or disorder is Vaginal Carcinoma, High Grade Ovarian Serous Adenocarcinoma, Malignant Ovarian Neoplasm, Soft Tissue Sarcoma, Gastric Adenocarcinoma, Vulvar Carcinoma, Malignant Solid Tumor, Osteosarcoma, Hepatocellular Carcinoma, Penile Carcinoma, Anal Carcinoma, Synovial Sarcoma, Non-Hodgkin Lymphoma, Multiple Myeloma, Malignant Breast Neoplasm, Malignant Lung Neoplasm, Breast Carcinoma, Histiocytic and Dendritic Cell Neoplasm, Malignant Pancreatic Neoplasm, or Malignant Ovarian Epithelial Tumor.

In certain embodiments, the disease or disorder is BCL-2. In some embodiments, the disease or disorder is mediated by BCL-2. In some embodiments, the disease or disorder is Non-Small Cell Lung Carcinoma, Burkitt Lymphoma, Small Cell Lung Carcinoma, Acute Lymphoblastic Leukemia, Follicular Lymphoma, Malignant Endometrial Neoplasm, Invasive Breast Carcinoma, Glioblastoma, Malignant Solid Tumor, Acute Myeloid Leukemia, Chronic Lymphocytic Leukemia, B-Cell Non-Hodgkin Lymphoma, Diffuse Large B-Cell Lymphoma Activated B-Cell Type, EBV-Positive Diffuse Large B-Cell Lymphoma, High Grade B-Cell Lymphoma, High Grade B-Cell Lymphoma with MYC and BCL2 and/or BCL6 Rearrangements, Triple-Hit Lymphoma, Chronic Myeloid Leukemia, Transformed Non-Hodgkin Lymphoma, Double-Hit Lymphoma, B-Cell Acute Lymphoblastic Leukemia, Hematopoietic and Lymphoid Malignancy, Diffuse Large B-Cell Lymphoma, Non-Hodgkin Lymphoma, Multiple Myeloma, B-Cell Lymphoma, Hodgkin Lymphoma, Diffuse Large B-Cell Lymphoma, Breast Carcinoma, Head and Neck Squamous Cell Carcinoma, Germinal Center B-Cell-Like Diffuse Large B-Cell Lymphoma, Lymphoma, or Mature B-Cell Lymphoma/Leukemia.

In some embodiments, the disease or disorder is associated with MCL-1. In some embodiments, the disease or disorder is mediated by MCL-1. In some embodiments, the disease or disorder is Melanoma, Malignant Breast Neoplasm, Lymphoma, Non-Small Cell Lung Carcinoma, Malignant Ovarian Neoplasm, Breast Carcinoma, Acute Lymphoblastic Leukemia, Head and Neck Squamous Cell Carcinoma, Hepatocellular Carcinoma, Malignant Prostate Neoplasm, Malignant Thyroid Gland Neoplasm, Pancreatic Ductal Adenocarcinoma, Malignant Colorectal Neoplasm, Malignant Solid Tumor, or Multiple Myeloma.

In some embodiments, the disease or disorder is associated with MYC. In certain embodiments, the disease or disorder is mediated by MYC. In some embodiments, the disease or disorder is Vaginal Carcinoma, Pancreatic Adenocarcinoma, Soft Tissue Sarcoma, Marginal Zone Lymphoma, Small Cell Lung Carcinoma, Follicular Lymphoma, Medulloblastoma, B-Cell Non-Hodgkin Lymphoma, Ewing Sarcoma, Small Lymphocytic Leukemia, Peripheral T-Cell Lymphoma, Transformed Non-Hodgkin Lymphoma, Double-Hit Lymphoma, Intraocular Lymphoma, Neuroblastoma, Merkel Cell Carcinoma, Head and Neck Squamous Cell Carcinoma, Myeloid Neoplasm, Adenocarcinoma of the Gastroesophageal Junction, Hodgkin Lymphoma, Mature B-Cell Non-Hodgkin Lymphoma, High Grade Ovarian Serous Adenocarcinoma, Gastric Squamous Cell Carcinoma, Acute Lymphoblastic Leukemia, Vulvar Carcinoma, Malignant Prostate Neoplasm, Glioblastoma, Esophageal Adenocarcinoma, Small Intestinal Lymphoma, Breast Angiosarcoma, Esophageal Squamous Cell Carcinoma, Diffuse Large B-Cell Lymphoma Activated B-Cell Type, EBV-Positive Diffuse Large B-Cell Lymphoma, High Grade B-Cell Lymphoma, High Grade B-Cell Lymphoma with MYC and BCL2 and/or BCL6 Rearrangements, Triple-Hit Lymphoma, Diffuse Large B-Cell Lymphoma, Chronic Lymphocytic Leukemia/Small Lymphocytic Lymphoma, B-Cell Lymphoma, Pancreatic Carcinoma, Endometrial Serous Adenocarcinoma, Pancreatic Ductal Adenocarcinoma, Lymphoma, Burkitt Lymphoma, Malignant Solid Tumor, Acute Myeloid Leukemia, Osteosarcoma, Ovarian Carcinoma, Leukemia, Bladder Carcinoma, Chronic Lymphocytic Leukemia, Penile Carcinoma, Anal Carcinoma, Sarcoma, Hematopoietic and Lymphoid Malignancy, Small Lymphocytic Lymphoma, Non-Hodgkin Lymphoma, Malignant Esophageal Neoplasm, Multiple Myeloma, Melanoma, Diffuse Large B-Cell Lymphoma, Malignant Breast Neoplasm, Breast Carcinoma, Germinal Center B-Cell-Like Diffuse Large B-Cell Lymphoma, Esophageal Adenosquamous Carcinoma, Non-Small Cell Lung Carcinoma, Malignant Ovarian Neoplasm, Prostate Carcinoma, Gastric Adenocarcinoma, Colorectal Carcinoma, Hairy Cell Leukemia, Gastric Adenosquamous Carcinoma, Brain Glioblastoma, Head and Neck Carcinoma, Esophageal Adenoid Cystic Carcinoma, Mantle Cell Lymphoma, Malignant Colorectal Neoplasm, Lymphoplasmacytic Lymphoma, Malignant Lung Neoplasm, or Mature B-Cell Lymphoma/Leukemia.

In some embodiments, the disease or disorder is associated with beta-catenin. In certain embodiments, the disease or disorder is mediated by beta-catenin. In some embodiments, the disease or disorder is Desmoplastic/Nodular Medulloblastoma, Non-Small Cell Lung Carcinoma, Colon Carcinoma, Gastric Adenocarcinoma, Colorectal Carcinoma, Medulloblastoma, Malignant Endometrial Neoplasm, Malignant Solid Tumor, Acute Myeloid Leukemia, Hepatocellular Carcinoma, Desmoid-Type Fibromatosis, Large Cell/Anaplastic Medulloblastoma, Colorectal Adenocarcinoma, Malignant Colon Neoplasm, Medulloblastoma with Extensive Nodularity, Malignant Colorectal Neoplasm, Intracranial Primitive Neuroectodermal Neoplasm, Malignant Lung Neoplasm, Primary Malignant Liver Neoplasm, or Pancreatic Ductal Adenocarcinoma.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Synthesis of Intermediates to Compounds of the Present Disclosure

### Synthetic Procedure of Intermediates Int-1 and Int-2

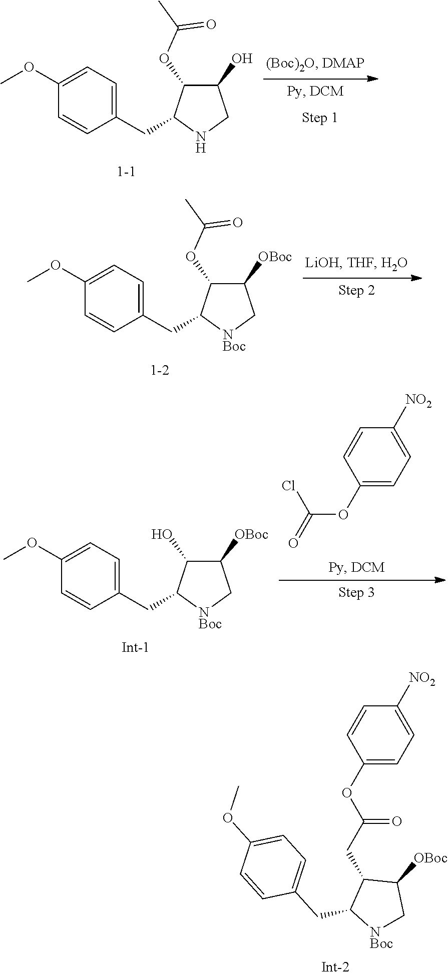

Step 1: To a stirred solution of anisomycin (5 g, 18.85 mmol, 1 eq.) in Pyridine (100 mL) was added Boc₂O (41.13 g, 188.46 mmol, 10 eq.) and DMAP (3.45 g, 28.27 mmol, 1.5 eq.) at room temperature. The resulting mixture was stirred over night at 25° C. Upon completion, the reaction mixture was concentrated in vacuo. The residue was purified by reversed-phase flash chromatography (0.05% NH₄HCO₃ in H₂O/ACN) to afford tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 91.2%) as a light yellow oil. MS: m/z: Calc'd for C₂₄H₃₅NO₈[M+H−56−100]⁺310, found 310.

Step 2: To a stirred solution of tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 17.18 mmol, 1 eq.) in THF (100 mL) was added LiOH (1.23 g, 51.55 mmol, 3 eq.) and H₂O (10 mL) at room temperature. The resulting mixture was stirred over night at 25° C. The resulting mixture was extracted with EtOAc (3×30 mL). The combined organic layers were washed with water (3×10 mL), dried over anhydrous Na₂SO₄. After filtration, the filtrate was concentrated under reduced pressure to afford crude product tert-butyl (2R,3S,4S)-4-[(tert-butoxycarbonyl)oxy]-3-hydroxy-2-[(4-methoxyphenyl)methyl]pyrrolidine-1-carboxylate (7.2 g, 98.9%) as a light yellow solid, which was used directly in the next step without further purification. MS: m/z: Calc'd for C₂₂H₃₃NO₇ [M+H−56−56]⁺312, found 312.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (4.7 g, 11.10 mmol, 1 eq.) and 4-nitrophenyl carbonochloridate (3.36 g, 16.67 mmol, 1.5 eq.) in DCM (50 mL) was added Pyridine (1.76 g, 22.25 mmol, 2 eq.) at room temperature. The resulting mixture was stirred at room temperature for another 2 h. After completion of reaction monitored by LCMS. The mixture was concentrated, the residue was purified by silica gel column chromatography, eluted with PE/EA (5:1) to afford tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (5.4 g, 82.7%) as a light yellow oil. MS: m/z: Calc'd for C₂₉H₃₆N₂O₁₁ [M+H−100]⁺489, found 489.

### Synthetic Procedure of Intermediates Int-3 and Int-4

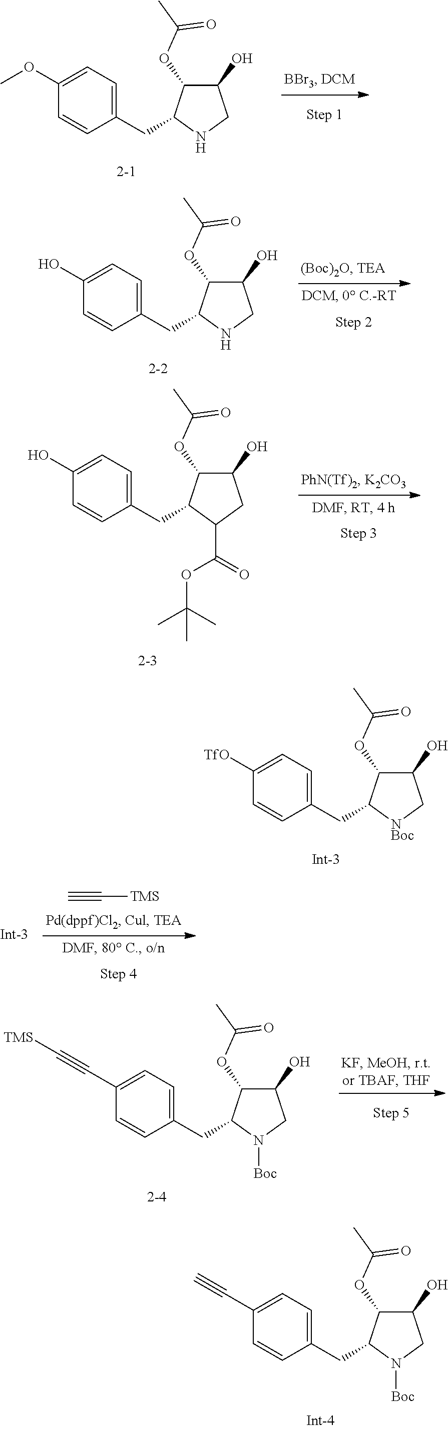

Step 1: To a stirred solution of anisomycin (1.5 g, 5.65 mmol, 1 equiv) in Dichloromethane (8 mL) was added Boron tribromide (16.8 mL, 3 equiv) at −78° C. The reaction mixture was stirred at −78° C. for 2 h and warmed to room temperature. The mixture was stirred for 1 h at room temperature and quenched by saturated NaHCO₃ solution. The DCM was removed, and the solution was lyophilized to obtain (2R,3S,4S)-4-hydroxy-2-[(4-hydroxyphenyl)methyl]pyrrolidin-3-yl acetate (1.8 g, 85.41%) as a crude. MS: m/z: Calc'd for C₁₃H₁NO₄ [M+H]⁺252; found 252.

Step 2: To a stirred solution of (2R,3S,4S)-4-hydroxy-2-[(4-hydroxyphenyl)methyl]pyrrolidin-3-yl acetate (1.8 g, 7.16 mmol, 1 equiv) and triethylamine (2.54 g, 25.07 mmol, 3.5 equiv) in DCM (30 mL) was added di-tert-butyl dicarbonate (1.88 g, 8.59 mmol, 1.2 equiv) at 0° C. The mixture was stirred at room temperature for 3 h. After completion of the reaction monitored by LCMS, the reaction mixture was filtrated. The filtrate was concentrated. The residue was purified by a reversed-phase column to obtain tert-butyl (2R,3S,4S)-3-(acetyloxy)-4-hydroxy-2-[(4-hydroxyphenyl)methyl]pyrrolidine-1-carboxylate (1.7 g, 67.54%) as a white solid. MS: m/z: Calc'd for C₁₈H₂₅NO₆ [M−H]⁻350; found 350.

Step 3: To a stirred solution of tert-butyl (2R,3S,4S)-3-(acetyloxy)-4-hydroxy-2-[(4-hydroxyphenyl)methyl]pyrrolidine-1-carboxylate (1.7 g, 4.83 mmol,) and Potassium carbonate (2.01 g, 14.51 mmol, 3 equiv) in DMF (16 mL) was added 1,1,1-trifluoro-N-phenyl-N-trifluoromethanesulfonylmethanesulfonamide (2.25 g, 6.28 mmol, 1.3 equiv) at 0° C. The reaction mixture was stirred at room temperature for 2 h. After completion of the reaction monitored by LCMS, the reaction mixture was filtrated. The filtrate was injected into a reversed-phase column and purified to obtain tert-butyl (2R,3S,4S)-3-(acetyloxy)-4-hydroxy-2-{[4-(trifluoromethanesulfonyloxy)phenyl]methyl}pyrrolidine-1-carboxylate (1.5 g, 64.13%) as a white solid. MS: m/z: Calc'd for C₁₉H₂₄F₃NO₈S [M+NH_{4]}⁺501; found 501.

Step 4: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (200 mg, 0.42 mmol, 1 equiv) and trimethylsilylacetylene (121.9 mg, 1.24 mmol, 3 equiv) in DMF (10 mL) was added TEA (167.4 mg, 1.65 mmol, 4 equiv), CuI (7.9 mg, 0.04 mmol, 0.1 equiv) and Pd(dppf)Cl₂·CH₂Cl₂ (50.6 mg, 0.06 mmol, 0.15 equiv) in portions at room. The reaction was placed under vacuum, sonicated and backfilled with nitrogen. The solution was stirred at 80° C. for 12 h. Desired product could be detected by LCMS. Water was used to quench the reaction, extracted with EA, concentrated, the residue was purified by Prep-TLC (PE/EA 2:1) to afford tert-butyl (2R,3S,4S)-3-(acetyloxy)-4-hydroxy-2-({4-[2-(trimethylsilyl)ethynyl]phenyl}methyl)pyrrolidine-1-carboxylate (160 mg, 89.6%) as a yellow oil. MS: m/z: Calc'd for C₂₃H₃₃NO₅Si[M−100]⁺332; found 332.

Step 5: A solution of tert-butyl (2R,3S,4S)-3-(acetyloxy)-4-hydroxy-2-({4-[2-(trimethylsilyl) ethynyl]phenyl}methyl)pyrrolidine-1-carboxylate (170 mg, 0.39 mmol, 1 equiv) and Triethylamine trihydrofluoride (190.5 mg, 1.18 mmol, 3.0 equiv) in THF (5 mL) was stirred at 60° C. for 12 h. Desired product could be detected by LCMS. Concentrated, the residue was purified by Prep-TLC (PE/EA 3:1) to afford tert-butyl (2R,3S,4S)-3-(acetyloxy)-2-[(4-ethynylphenyl)methyl]-4-hydroxypyrrolidine-1-carboxylate (135 mg, 95.36%) as a light yellow solid. MS: m/z: Calc'd for C₂₀H₂₅NO₅ [M−56]⁺304; found 304.

### Synthetic Procedure of Intermediate Int-5

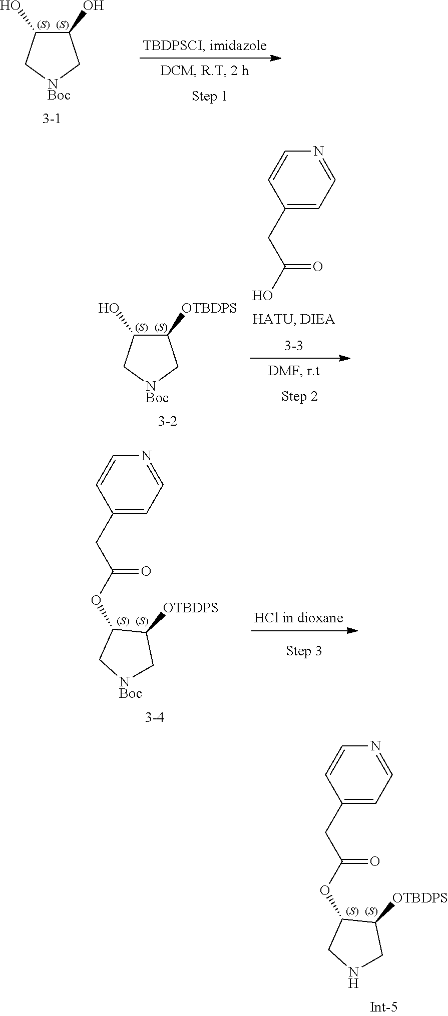

Step 1: To a stirred solution of tert-butyl (3S,4S)-3,4-dihydroxypyrrolidine-1-carboxylate (3-1, 1 g, 4.92 mmol, 1 eq.), Imidazole (1 g, 14.76 mmol, 3 eq.) and TBDPSCl (1.2 g, 4.43 mmol, 0.9 eq.) in DCM (10 mL) was stirred for 1 h at room temperature. Desired product could be detected by LCMS. Concentrated, the residue was purified by silica gel column chromatography, eluted with PE/EA (2:1) to afford tert-butyl (3S,4S)-3-[(tert-butyldiphenylsilyl)oxy]-4-hydroxypyrrolidine-1-carboxylate (1.3 g, 59.8%) as a colorless oil. MS: m/z: Calc'd for C₂₅H₃₅NO₄Si[M−H]⁻440; found 440.

Step 2: To a solution of tert-butyl (3S,4S)-3-[(tert-butyldiphenylsilyl)oxy]-4-hydroxypyrrolidine-1-carboxylate (3-2, 1.3 g, 2.94 mmol, 1 eq.) in DCM (15 mL) was added 4-acetylpyridine (3-3, 0.6 g, 4.42 mmol, 1.5 eq.), DCC (0.9 g, 4.42 mmol, 1.5 eq.) and DMAP (360 mg, 2.94 mmol, 1 eq.). The mixture was stirred at room temperature for 1 h. Desired product could be detected by LCMS. The resulting mixture was filtered, the filter cake was washed with ethyl acetate (3×5 mL). The filtrate was concentrated under reduced pressure. The residue was purified by silica gel column chromatography, eluted with PE/EA (1:1) to afford tert-butyl (3S,4S)-3-[(tert-butyldiphenylsilyl)oxy]-4-{[2-(pyridin-4-yl)acetyl]oxy}pyrrolidine-1-carboxylate (1.6 g, 96.9%) as a colorless oil. MS: m/z: Calc'd for C₃₂H₄₀N₂O₅Si[M−56]⁺505; found 505.

Step 3: To a solution of tert-butyl (3S,4S)-3-[(tert-butyldiphenylsilyl)oxy]-4-{[2-(pyridin-4-yl)acetyl]oxy}pyrrolidine-1-carboxylate (1.6 g, 2.85 mmol) in dioxane (15 mL) was added HCl(gas) in 1,4-dioxane (15 mL). The mixture was stirred at room temperature for 1 h. Desired product could be detected by LCMS. The mixture was concentrated under reduced pressure to afford crude product which was used in the next step directly without further purification. MS: m/z: Calc'd for C₂₇H₃₂N₂O₃Si[M+H]⁺461, found 461.

### Example 2: Synthesis of Compounds of the Present Disclosure

General Procedure I: Condensation Reaction with DCC and DMAP

To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int-1, 0.24 mmol, 1 eq.) and corresponding acid (2 eq.) in DCM (5 mL) was added DCC (1.5 eq.) and DMAP (1 eq.). The reaction mixture was stirred at ambient temperature for 2 h. Upon completion, the reaction mixture was quenched with water and extracted with EA. The extracts were washed with brine, dried over anhydrous sodium sulfate, filtered and concentrated. The residue was purified by reversed column chromatography.

### General Procedure II: Substitution Reaction for Synthesis of Carbamate

To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2, 0.12 mmol, 1 eq.) and corresponding amine (1 eq.) in ACN (5 mL) was added DIEA (3 eq.) at room temperature. The resulting mixture was stirred at room temperature for 12 h. After completion, the reaction mixture was concentrated in vacuo. The residue was purified by reversed column chromatography.

### General Procedure III: Boc Deprotection

The corresponding Boc-protected amine (1 equiv) was dissolved in anhydrous CH₂Cl₂ (5 mL/mmol), and TFA (5 mL/mmol) was added. The mixture was stirred at r.t. for 2 h. After removal of the volatiles, the oily residue was further dried under high vacuum. The residue was purified by prep-HPLC.

General Procedure IV: Reductive Amination with AcOH and NaBH(OAc)₃

To a stirred solution of (3S,4S)-4-[(tert-butyldiphenylsilyl)oxy]pyrrolidin-3-yl 2-(pyridin-4-yl)acetate (Int-5, 0.16 mmol, 1 eq.) and corresponding benzaldehyde (1.5 eq.) in DCM (2 mL) was added DIEA (2 eq.) and AcOH (2 eq.), the resulting mixture was stirred at room temperature for 1 h. Then NaBH(OAc)₃ (2 eq.) was added at 0° C. and the resulting mixture was stirred at RT for 1 h. Upon completion, the mixture was cooled to r.t., concentrated in vacuo. The residue was purified by reversed-phase flash chromatography.

### General Procedure V: TBDPS Deprotection

The corresponding TBDPS-protected alcohol (1 equiv) was dissolved in anhydrous THF (5 mL/mmol), and TBAF (1 equiv) was added at 0° C. The mixture was stirred at r.t. for 2 h. Upon completion, the mixture was concentrated in vacuo. The residue was purified by prep-HPLC.

### General Procedure VI: Cyclization Reaction for Synthesis of Substituted Triazoles

To a stirred solution of tert-butyl (2R,3S,4S)-4-[(tert-butoxycarbonyl)oxy]-2-[(4-methoxyphenyl)methyl]-3-(prop-2-yn-1-yloxy)pyrrolidine-1-carboxylate (Int-4, 80 mg, 0.17 mmol, 1 eq.) and corresponding azide (1 eq.) in MeOH (4 mL) was added CuSO_{4·5}H₂O (1 eq.) and sodium (5R)-5-[(1S)-1,2-dihydroxyethyl]-3,4-dihydroxy-2,5-dihydrofuran-2-one (2 eq.) at 0° C. The resulting mixture was stirred at room temperature for overnight. Desired product could be detected by LCMS. The resulting mixture was concentrated under reduced pressure. The residue was purified by reversed-phase flash chromatography.

### Synthetic Procedure of V0081

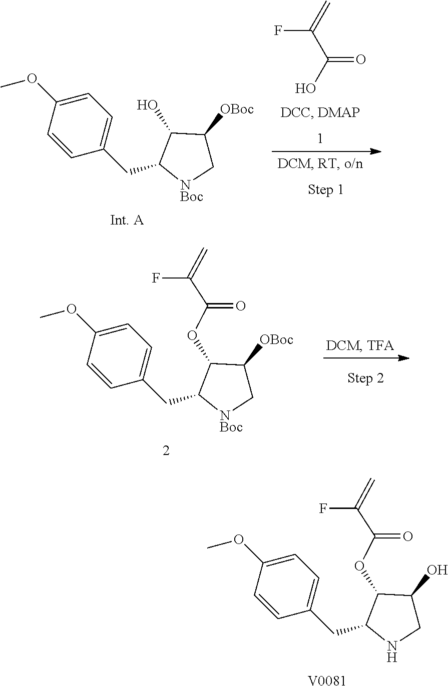

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and 2-fluoroprop-2-enoic acid (51.0 mg, 0.56 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-[(2-fluoroprop-2-enoyl)oxy]-2-[(4-methoxyphenyl)methyl]pyrrolidine-1-carboxylate (35 mg, 24.9%) as a white solid. MS: m/z: Calc'd for C₂₅H₃₄FNO₈[M+H−56−56]⁺384; Found 384.

Step 2: To a stirred mixture of tert-butyl (2R,3S,4S)-4-[(tert-butoxycarbonyl)oxy]-3-[(2-fluoroprop-2-enoyl)oxy]-2-[(4-methoxyphenyl)methyl]pyrrolidine-1-carboxylate (30 mg, 0.06 mmol) in DCM (3 mL) was added TFA (1 mL) dropwise at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-fluoroprop-2-enoate (12.6 mg, 69.4%) as a white solid. MS: m/z: Calc'd for C₁₅H₁₈FNO₄ [M+H]⁺296; Found 296. ¹H NMR (400 MHz, DMSO-d₆) δ 9.69 (s, 1H), 7.20 (d, J=8.3 Hz, 2H), 6.92 (d, J=8.3 Hz, 2H), 6.16 (d, J=4.1 Hz, 1H), 5.80 (dd, J=14.3, 4.1 Hz, 1H), 4.99 (d, J=3.2 Hz, 1H), 4.34 (d, J=4.3 Hz, 1H), 4.10 (s, 1H), 3.77 (s, 3H), 3.54 (s, 2H), 3.11 (d, J=12.8 Hz, 1H), 3.05-2.90 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.5

### Synthetic Procedure of V0091

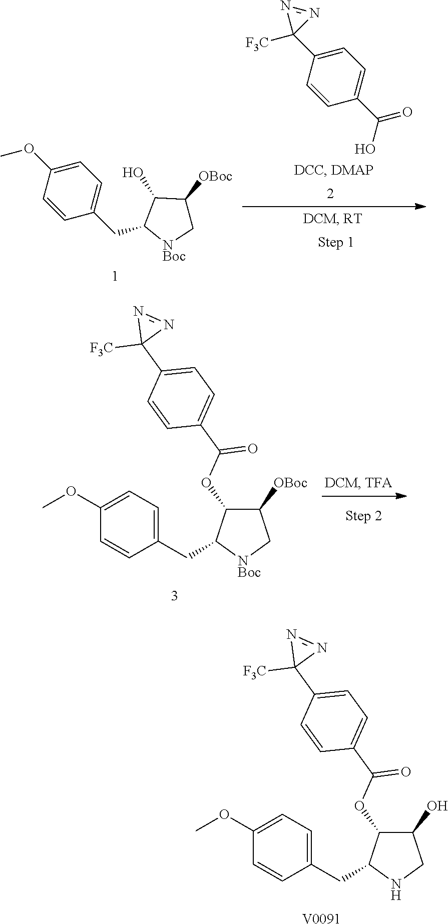

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and 4-[3-(trifluoromethyl)diazirin-3-yl]benzoic acid (130.42 mg, 0.566 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-2-[(4-methoxyphenyl)methyl]-3-{4-[3-(trifluoromethyl)diazirin-3-yl]benzoyloxy}pyrrolidine-1-carboxylate (130 mg, 72.1%) as a white solid. MS: m/z: Calc'd for C₃₁H₃₆F₃N₃O₈ [M+Na+MeCN]⁺699; Found 699.

Step 2: To a stirred mixture of tert-butyl (2R,3S,4S)-4-[(tert-butoxycarbonyl)oxy]-2-[(4-methoxyphenyl)methyl]-3-{4-[3-(trifluoromethyl)diazirin-3-yl]benzoyloxy}pyrrolidine-1-carboxylate (125 mg, 0.197 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 4-[3-(trifluoromethyl)diazirin-3-yl]benzoate (27.4 mg, 31.9%) as a white solid. MS: m/z: Calc'd for C₂₁H₂₀F₃N₃O₄[M+H]⁺436; Found 436. ¹H NMR (400 MHz, DMSO-d₆) δ 9.59 (s, 1H), 9.51 (s, 1H), 8.26 (dd, J=8.6, 1.9 Hz, 2H), 7.50 (d, J=8.6 Hz, 2H), 7.21-7.13 (m, 2H), 6.92-6.84 (m, 2H), 6.13 (s, 1H), 5.14-5.09 (m, 1H), 4.37 (d, J=4.3 Hz, 1H), 4.13 (s, 1H), 3.72 (s, 3H), 3.60 (s, 1H), 3.16-2.95 (m, 3H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 16% B to 46% B in 10 min; Wave Length: 254 nm/220 nm nm; RT1(min): 8.5

### Synthetic Procedure of V0092

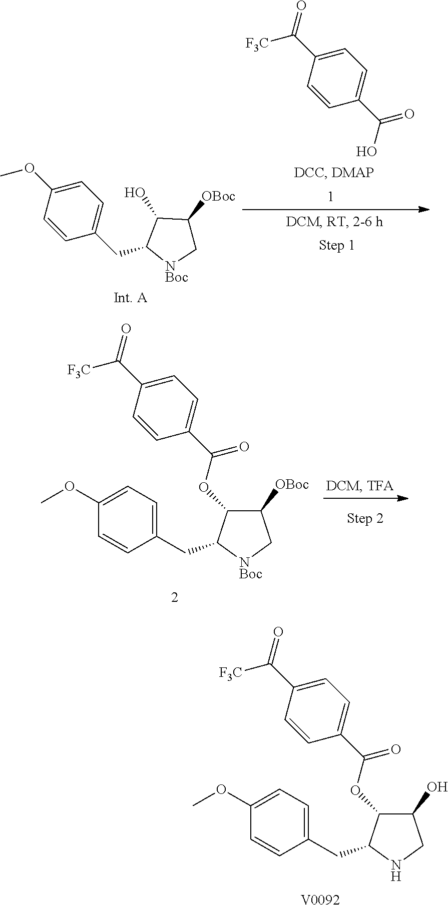

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and 4-(2,2,2-trifluoroacetyl)benzoic acid (123.61 mg, 0.566 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-2-[(4-methoxyphenyl)methyl]-3-[4-(2,2,2-trifluoroacetyl)benzoyloxy]pyrrolidine-1-carboxylate (130 mg, 73.5%) as a white solid. MS: m/z: Calc'd for C₃₁H₃₆F₃NO₉ [M+H−56−100]⁺468; Found 468.

Step 2: To a stirred mixture of tert-butyl (2R,3S,4S)-4-[(tert-butoxycarbonyl)oxy]-2-[(4-methoxyphenyl)methyl]-3-[4-(2,2,2-trifluoroacetyl)benzoyloxy]pyrrolidine-1-carboxylate (115 mg, 0.18 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 4-(2,2,2-trifluoroacetyl)benzoate (27.9 mg, 35.3%) as a white solid. MS: m/z: Calc'd for C₂₁H₂₀F₃NO₅ [M+H]⁺424; Found 424. ¹H NMR (400 MHz, DMSO-d₆) δ 8.22-8.15 (m, 2H), 7.86-7.77 (m, 2H), 7.22-7.14 (m, 2H), 6.93-6.85 (m, 2H), 5.15-5.10 (m, 1H), 4.36 (d, J=4.1 Hz, 1H), 4.13 (s, 1H), 3.72 (s, 3H), 3.62 (d, J=12.1 Hz, 1H), 3.17-3.03 (m, 1H), 3.01-2.99 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 3% B to 33% B in 10 min; Wave Length: 254 nm/220 nm nm; RT1(min): 8.5

### Synthetic Procedure of V0106

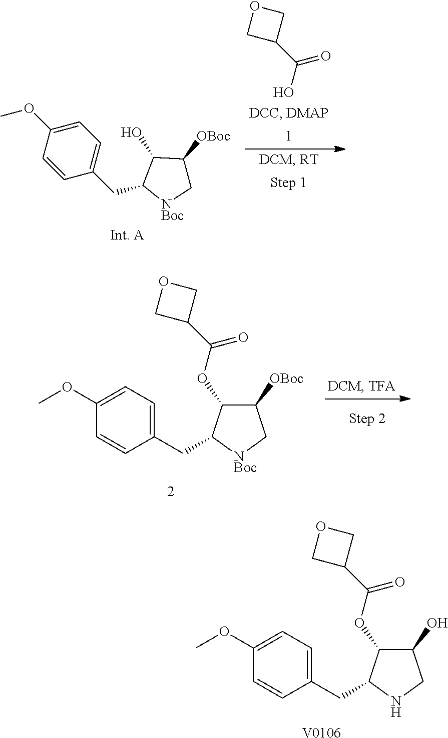

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and oxetane-3-carboxylic acid (57.8 mg, 0.56 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-(oxetane-3-carbonyloxy)pyrrolidine-1-carboxylate (70 mg, 48.6%) as a white solid. MS: m/z: Calc'd for C₂₆H₃₇NO₉ [M+H−56−56]⁺396; Found 396.

Step 2: To a stirred mixture of tert-butyl (2R,3S,4S)-4-[(tert-butoxycarbonyl)oxy]-2-[(4-methoxyphenyl)methyl]-3-(oxetane-3-carbonyloxy)pyrrolidine-1-carboxylate (65 mg, 0.128 mmol) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl oxetane-3-carboxylate (18.9 mg, 47.2%) as a white solid. MS: m/z: Calc'd for C₁₆H₂₁NO₅ [M+H]⁺308; Found 308. MS: m/z: Calc'd for C₁₅H₁₈FNO₄ [M+H]⁺296, found 296. ¹H NMR (400 MHz, DMSO-d₆) δ 8.34 (s, 1H), 7.16 (d, J=8.3 Hz, 2H), 6.88 (d, J=8.2 Hz, 2H), 4.84 (s, 1H), 4.79 (td, J=8.9, 6.1 Hz, 2H), 4.67 (t, J=6.2 Hz, 2H), 4.15 (s, 1H), 4.00-3.95 (m, 1H), 3.73 (dd, J=11.4, 3.0 Hz, 1H), 3.73 (s, 3H), 3.67 (s, 1H), 2.82 (s, 3H). Prep-HPLC-conditions: Column: Xselect CSH Prep C18 C18 Column, 30*150 mm, 5 μm; Mobile Phase A: Water(0.05% FA), Mobile Phase B: ACN; Flow rate: 60 mL/min mL/min; Gradient: 10% B to 30% B in 10 min; Wave Length: 254 nm/220 nm nm; RT1(min): 8.5

### Synthetic Procedure of V0107

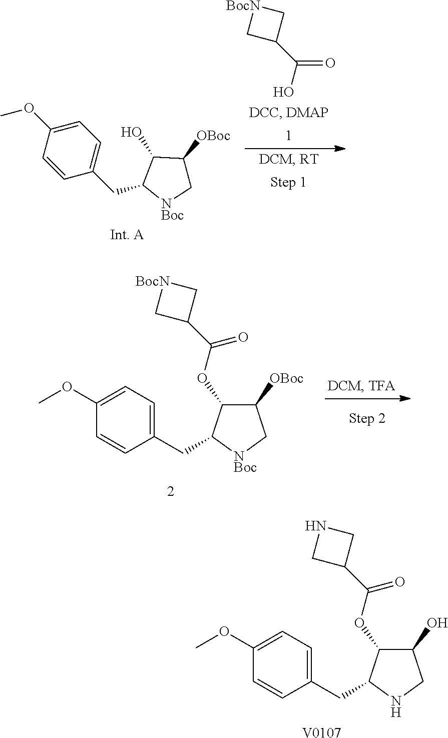

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and 1-(tert-butoxycarbonyl)azetidine-3-carboxylic acid (114.0 mg, 0.56 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford 3-(2R,3S,4S)-1-(tert-butoxycarbonyl)-4-[(tert-butoxycarbonyl)oxy]-2-[(4-methoxyphenyl)methyl]pyrrolidin-3-yl 1-tert-butyl azetidine-1,3-dicarboxylate (100 mg, 58.17%) as a white solid. MS: m/z: Calc'd for C₃₁H₄₆N₂O₁₀ [M+Na]⁺629; Found 629.

Step 2: To a stirred mixture of 3-(2R,3S,4S)-1-(tert-butoxycarbonyl)-4-[(tert-butoxycarbonyl)oxy]-2-[(4-methoxyphenyl)methyl]pyrrolidin-3-yl 1-tert-butyl azetidine-1,3-dicarboxylate (110 mg, 0.18 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl azetidine-3-carboxylate (29.9 mg, 60.0%) as a white solid. MS: m/z: Calc'd for C₁₆H₂₂N₂O₄ [M+H]⁺307; Found 307. ¹H NMR (400 MHz, DMSO-d₆) δ 9.55 (s, 4H), 7.23 (d, J=8.4 Hz, 2H), 6.90 (d, J=8.3 Hz, 2H), 6.14-6.09 (m, 1H), 4.94 (d, J=3.2 Hz, 1H), 4.35-4.30 (m, 1H), 4.25-4.09 (m, 4H), 4.03-3.95 (m, 1H), 3.90-3.77 (m, 1H), 3.73 (s, 3H), 3.52 (dd, J=12.6, 4.5 Hz, 1H), 3.07 (d, J=12.6 Hz, 1H), 3.02-2.87 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 25% B in 10 min; Wave Length: 254 nm/220 nm nm; RT1(min): 8.5

### Synthetic Procedure of V0108

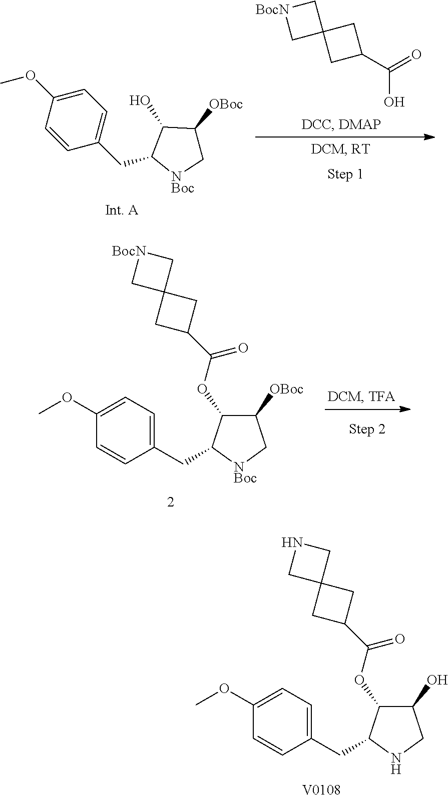

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 2-(tert-butoxycarbonyl)-2-azaspiro[3.3]heptane-6-carboxylic acid (113.9 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford 6-(2R,3S,4S)-1-(tert-butoxycarbonyl)-4-[(tert-butoxycarbonyl)oxy]-2-[(4-methoxyphenyl)methyl]pyrrolidin-3-yl 2-tert-butyl 2-azaspiro[3.3]heptane-2,6-dicarboxylate (110 mg, 72.0%) as a white solid. MS: m/z: Calc'd for C₃₄H₅₀N₂O₁₀ [M+Na]⁺669; Found 669.

Step 2: To a stirred mixture of 6-(2R,3S,4S)-1-(tert-butoxycarbonyl)-4-[(tert-butoxycarbonyl)oxy]-2-[(4-methoxyphenyl)methyl]pyrrolidin-3-yl 2-tert-butyl 2-azaspiro[3.3]heptane-2,6-dicarboxylate (105 mg, 0.16 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 2-azaspiro[3.3]heptane-6-carboxylate as a white solid. MS: m/z: Calc'd for C₁₉H₂₆N₂O₄ [M+H]⁺347; Found 347. ¹H NMR (400 MHz, DMSO-d₆) δ 7.19-7.17 (m, 2H), 6.95-6.87 (m, 2H), 4.88 (d, J=3.6 Hz, 1H), 4.21 (d, J=3.6 Hz, 1H), 4.04 (dd, J=7.8, 3.6 Hz, 1H), 4.02 (s, 4H), 3.94 (s, 3H), 3.77 (d, J=17.6 Hz, 1H), 3.42-3.40 (m, 2H), 3.13 (dd, J=16.0, 7.8 Hz, 2H), 2.61-2.54 (m, 1H), 2.52-2.46 (m, 1H), 2.49-2.41 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 3% B to 33% B in 10 min; Wave Length: 254 nm/220 nm nm; RT1(min): 8.5

### Synthetic Procedure of V0109

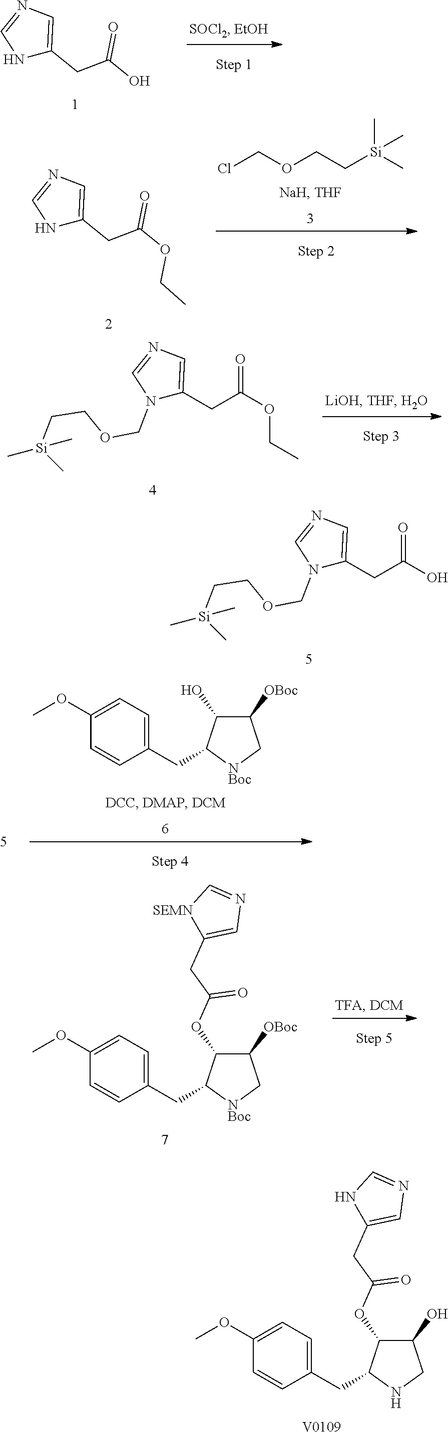

Step 1: To a solution of 2-(1H-imidazol-5-yl)acetic acid (900 mg, 7.13 mmol, 1 equiv) in EtOH (30 mL) was added SOCl₂ (5 mL), the mixture was stirred at 70° C. for 4 h. LCMS showed the reaction was completed. The reaction was concentrated and purified by reversed-phase flash chromatography to afford ethyl 2-(3H-imidazol-4-yl)acetate (700 mg, 63.6%) as colorless oil. MS: m/z: Calc'd for C₇H₁₀N₂O₂[M+H]⁺155; Found, 155.

Step 2: To the solution of ethyl 2-(3H-imidazol-4-yl)acetate (850 mg, 5.51 mmol, 1 equiv) in DCM (60 mL) was added NaH (60%) (550 mg, 13.75 mmol, 2.50 equiv) at 0° C., the mixture was stirred at 0° C. for 0.5 h, then [2-(chloromethoxy)ethyl]trimethylsilane (1838 mg, 11.02 mmol, 2 equiv) was added, the mixture solution was stirred at room temperature overnight. LCMS showed the reaction was completed. The reaction was quenched with NH₄Cl solution at 0° C., and extracted by EA, dried, then purified by reversed-phase flash chromatography (0.05% TFA) to afford ethyl 2-(3-{[2-(trimethylsilyl)ethoxy]methyl}imidazol-4-yl)acetate (400 mg, 25.5%). MS: m/z: Calc'd for C₁₇H₂₄N₂O₃Si[M+H]⁺285; Found, 285.

Step 3: To the solution of ethyl 2-(3-{[2-(trimethylsilyl)ethoxy]methyl}imidazol-4-yl)acetate (400 mg, 1.406 mmol, 1 equiv) in THF (20 mL), MeOH (5 mL) was added LiOH·H₂O (177.20 mg, 4.218 mmol, 3.0 equiv) in H₂O (5 mL). The mixture solution was stirred at room temperature overnight. LCMS showed the reaction was completed, concentrated the mixture solution and diluted with H₂O (8 ml), and neutralized to pH=7 with 1N HCl. The residue was purified by reversed phase flash to afford (3-{[2-(trimethylsilyl)ethoxy]methyl}imidazol-4-yl)acetic acid (350 mg, 97.07%) as colorless oil. MS: m/z: Calc'd for C₁₁H₂₀N₂O₃Si[M+H]⁺257; Found, 257

Step 4: To the solution of (3-{[2-(trimethylsilyl)ethoxy]methyl}imidazol-4-yl)acetic acid (60 mg, 0.23 mmol, 2 equiv) in DCM (8 mL) were added DCC (36 mg, 0.17 mmol, 1.5 equiv), DMAP (17.3 mg, 0.14 mmol, 1.2 equiv), tert-butyl (2R,3S,4S)-4-[(tert-butoxycarbonyl) oxy]-3-hydroxy-2-[(4-methoxyphenyl)methyl]pyrrolidine-1-carboxylate (50 mg, 0.11 mmol, 1.00 equiv) at 0° C., the mixture solution was stirred at room temperature overnight, the reaction was concentrated and purified by TLC to afford tert-butyl (2R,3S,4S)-4-[(tert-butoxycarbonyl) oxy]-2-[(4-methoxyphenyl)methyl]-3-{[2-(3-{[2-(trimethylsilyl)ethoxy]methyl}imidazol-4-yl)acetyl]oxy}pyrrolidine-1-carboxylate (60 mg, 76.7%) as colorless oil. MS: m/z: Calc'd for C₃₃H₅₁N₃O₉Si[M+H]⁺662; Found, 662

Step 5: To the solution of tert-butyl (2R,3S,4S)-4-[(tert-butoxycarbonyl)oxy]-2-[(4-methoxy phenyl)methyl]-3-{[2-(3-{[2-(trimethylsilyl)ethoxy]methyl}imidazol-4-yl)acetyl]oxy}pyrrolidine-1-carboxylate (65 mg, 0.09 mmol, 1 equiv) in DCM (10 mL) was added TFA (2 mL), the mixture was stirred at room temperature overnight. Upon completion, the reaction mixture was concentrated in vacuo. The crude was purified by prep-HPLC to afford (2R,3S,4S)-4-hydroxy-2-[(4-methoxyphenyl)methyl]pyrrolidin-3-yl 2-(3H-imidazol-4-yl)acetate (26.8 mg, 60.7%) as white solid. ¹H NMR (400 MHz, DMSO-d₆) δ 9.03 (d, J=1.4 Hz, 1H), 7.58 (d, J=1.4 Hz, 1H), 7.24-7.16 (m, 2H), 6.94-6.85 (m, 2H), 4.96-4.94 (m, 1H), 4.29-4.24 (m, 1H), 4.12-3.96 (m, 3H), 3.73 (s, 3H), 3.49-3.43 (m, 1H), 3.14-3.08 (m, 1H), 2.97-2.81 (m, 2H).

Prep-HPLC-conditions: Column: XBridge Prep Shield RP18 OBD C18 Column, 30*150 mm, 5 μm; Mobile Phase A: Water (0.1% FA), Mobile Phase B: ACN; Flow rate: 60 mL/min mL/min; Gradient: 2% B to 15% B in 10 min; Wave Length: 254 nm/220 nm nm; RT1(min): 6.2

### Synthetic Procedure of V0110

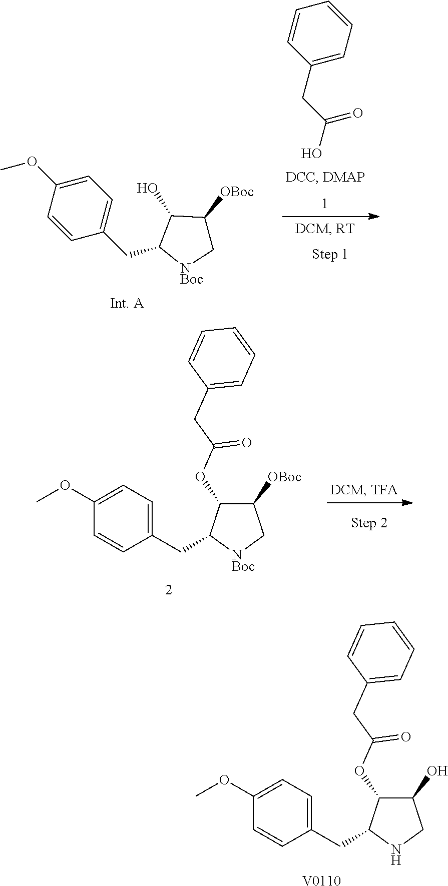

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and o-phenylacetic acid (77.16 mg, 0.566 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl) methyl]-3-[(2-phenylacetyl)oxy]pyrrolidine-1-carboxylate (120 mg, 78.1%) as a white solid. MS: m/z: Calc'd for C₃₀H₃₉NO₈[M+H−56−56]⁺436; Found 436.

Step 2: To a stirred mixture of tert-butyl (2R,3S,4S)-4-[(tert-butoxycarbonyl)oxy]-2-[(4-methoxyphenyl)methyl]-3-[(2-phenylacetyl)oxy]pyrrolidine-1-carboxylate (115 mg, 0.21 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-phenylacetate (27.9 mg, 36.9%) as a white solid. MS: m/z: Calc'd for C₂₀H₂₃NO₄ [M+H]⁺342; Found 342. ¹H NMR (400 MHz, DMSO-d₆) δ 9.53 (s, 1H), 9.29 (s, 1H), 7.43-7.32 (m, 5H), 7.03 (d, J=8.8 Hz, 2H), 6.87-6.79 (m, 2H), 6.03 (d, J=3.5 Hz, 1H), 4.82 (d, J=3.4 Hz, 1H), 4.20 (d, J=4.0 Hz, 1H), 4.00-3.98 (m, 1H), 3.82-3.80 (m, 1H), 3.75-3.72 (m, 1H), 3.72 (s, 3H), 3.49-3.41 (m, 1H), 3.09-3.07 (m, 1H), 2.85 (d, J=7.7 Hz, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.5

### Synthetic Procedure of VO111

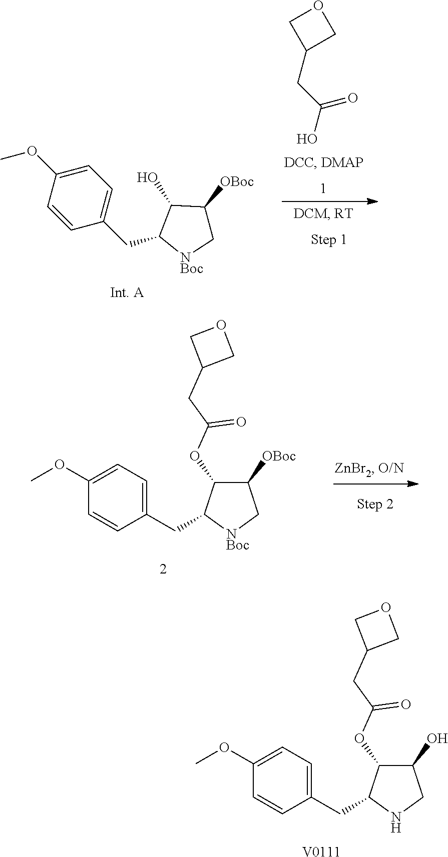

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 150 mg, 0.35 mmol, 1 eq.) and oxetan-3-ylacetic acid (82.2 mg, 0.71 mmol, 2 eq.) in DCM (5 mL) were added DCC (109.6 mg, 0.53 mmol, 1.5 eq.) and DMAP (43.2 mg, 0.35 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl) methyl]-3-{[2-(oxetan-3-yl)acetyl]oxy}pyrrolidine-1-carboxylate (140 mg, 75.7%) as a white solid. MS: m/z: Calc'd for C₂₇H₃₉NO₉ [M+Na]⁺544; Found 544.

Step 2: To a stirred solution of tert-butyl (2R,3S,4S)-4-[(tert-butoxycarbonyl)oxy]-2-[(4-methoxyphenyl)methyl]-3-{[2-(oxetan-3-yl)acetyl]oxy}pyrrolidine-1-carboxylate (140 mg, 0.27 mmol, 1 eq.) in DCM (5 mL) was added ZnBr₂ (604.4 mg, 2.68 mmol, 10 eq.) at 0° C. under nitrogen atmosphere. The resulting mixture was stirred for overnight at room temperature under nitrogen atmosphere. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(oxetan-3-yl)acetate (24.1 mg, 27.8%) as a white solid. MS: m/z: Calc'd for C₁₇H₂₃NO₅ [M+H]⁺322; found 322. ¹H NMR (400 MHz, DMSO-d₆) δ 7.18-7.10 (m, 2H), 6.89-6.82 (m, 2H), 4.76-4.65 (m, 3H), 4.32-4.25 (m, 2H), 4.02 (s, 1H), 3.80-3.75 (m, 3H), 3.65-3.55 (m, 1H), 3.33-3.22 (m, 3H), 2.76-2.65 (m, 5H). Prep-HPLC-conditions: Column: XselectCSH Prep OBD C18 Column, 30*150 mm, 5 μm; Mobile Phase A: Water (0.1% FA), Mobile Phase B: ACN; Flow rate: 60 mL/min mL/min; Gradient: 2% B to 25% B in 8 min; Wave Length: 254 nm/220 nm nm; RT1(min): 6.55

### Synthetic Procedure of V0112

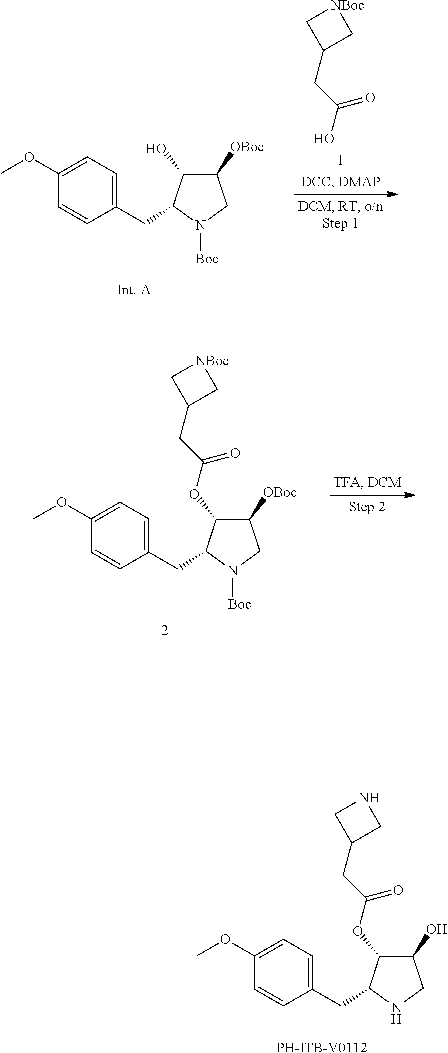

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and [1-(tert-butoxycarbonyl)azetidin-3-yl]acetic acid (91.5 mg, 0.42 mmol, 1.5 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at room temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-3-({2-[1-(tert-butoxycarbonyl)azetidin-3-yl]acetyl}oxy)-4-[(tert-butoxycarbonyl)oxy]-2-[(4-methoxyphenyl) methyl]pyrrolidine-1-carboxylate (160 mg, 91.0%) as a white solid. MS: m/z: Calc'd for C₃₂H₄₈N₂O₁₀ [M+H⁺22]⁺643, found 643.

Step 2: To a stirred mixture of tert-butyl (2R,3S,4S)-3-({2-[1-(tert-butoxycarbonyl)azetidin-3-yl]acetyl}oxy)-4-[(tert-butoxycarbonyl)oxy]-2-[(4-methoxyphenyl)methyl]pyrrolidine-1-carboxylate (80 mg, 0.13 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(azetidin-3-yl)acetate; trifluoroacetic acid (27.7 mg, 48.3%) as a yellow oil. MS: m/z: Calc'd for C₁₇H₂₄N₂O₄ [M+H]⁺321, found 321. ¹H NMR (400 MHz, DMSO-d₆) δ 7.23-7.12 (m, 2H), 6.92-6.83 (m, 2H), 4.85 (d, J=3.5 Hz, 1H), 4.20 (d, J=4.2 Hz, 1H), 4.06-3.98 (m, 3H), 3.79-3.73 (m, 2H), 3.41 (dd, J=12.8, 4.4 Hz, 1H), 3.18-3.05 (m, 2H), 2.96-2.76 (m, 4H). Prep-HPLC-conditions: Flow rate: 60 mL/min mL/min; Gradient: 2% B to 21% B in 10 min; Wave Length: 254 nm/220 nm nm; RT1(min): 8.5

### Synthetic Procedure of V0113

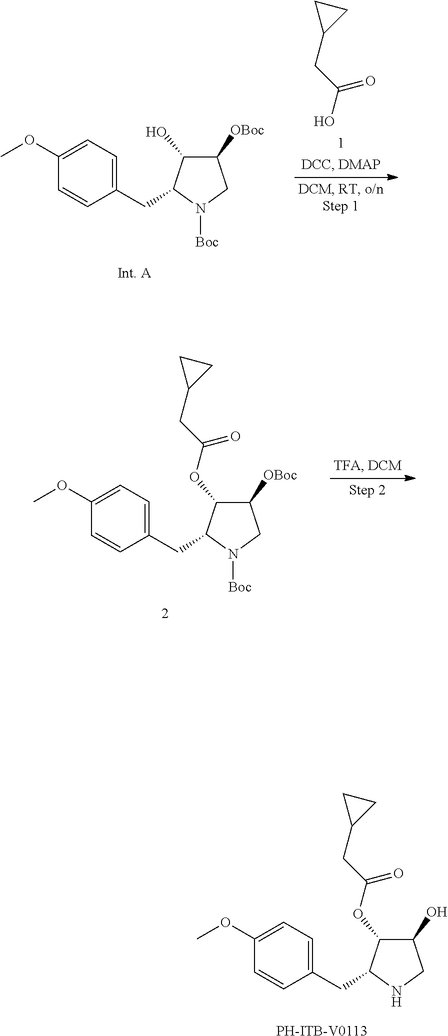

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1 eq.) and cyclopropylacetic acid (42.6 mg, 0.42 mmol, 1.5 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-[(2-cyclopropylacetyl)oxy]-2-[(4-methoxyphenyl)methyl]pyrrolidine-1-carboxylate (120 mg, 83.8%) as a white solid. MS: m/z: Calc'd for C₂₇H₃₉NO₈[M+H−100−56]⁺350, found 350.

Step 2: To a stirred mixture of tert-butyl (2R,3S,4S)-4-[(tert-butoxycarbonyl)oxy]-3-[(2-cyclopropylacetyl)oxy]-2-[(4-methoxyphenyl)methyl]pyrrolidine-1-carboxylate hydrofluoride (80 mg, 0.15 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-cyclopropylacetate; trifluoroacetic acid (37.1 mg, 56.8%) as a white solid. MS: m/z: Calc'd for C₁₇H₂₃NO₄ [M+H]⁺306, found 306. ¹H NMR (400 MHz, DMSO-d₆) δ 7.22 (d, J=8.6 Hz, 2H), 6.95-6.87 (m, 2H), 6.03 (d, J=3.4 Hz, 1H), 4.90 (d, J=3.4 Hz, 1H), 4.20 (d, J=4.1 Hz, 1H), 4.02-3.98 (m, 1H), 3.74 (s, 3H), 3.46 (dd, J=12.7, 4.4 Hz, 1H), 3.08 (d, J=12.6 Hz, 1H), 3.01-2.86 (m, 2H), 2.41-2.26 (m, 2H), 1.10-1.02 (m, 1H), 0.57-0.47 (m, 2H), 0.21 (dd, J=4.8, 1.5 Hz, 2H). Prep-HPLC-conditions: Flow rate: 60 mL/min mL/min; Gradient: 4% B to 34% B in 10 min; Wave Length: 254 nm/220 nm nm; RT1(min): 8.5

### Synthetic Procedure of V000144

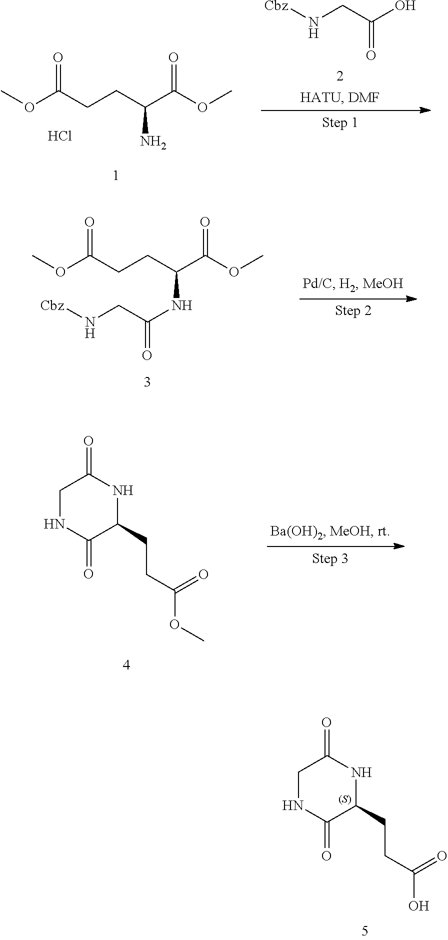

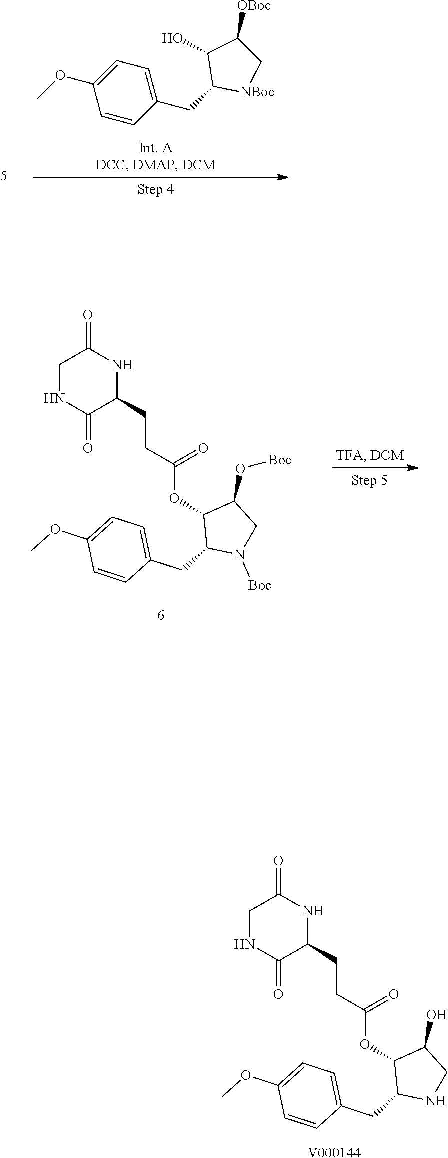

Step 1: To a stirred solution of dimethyl glutamic acid (5 g, 28.541 mmol, 1 equiv) and (cbz)gly (5.97 g, 28.54 mmol, 1.0 eq.) in DMF (50 mL) were added HATU (16.28 g, 42.81 mmol, 1.5 eq.) and DIEA (11.07 g, 85.62 mmol, 3.0 eq.) at 0° C. The resulting reaction mixture was stirred at ambient temperature for overnight under nitrogen atmosphere. Upon completion, the mixture was purified directly by reversed-phase flash chromatography (0.05% TFA in water and acetonitrile) to afford 1,5-dimethyl (2S)-2-(2-{[(benzyloxy)carbonyl]amino}acetamido) pentanedioate (7.86 g, 75.2%) as a yellow oil. MS: m/z: Calc'd for C₁₇H₂₂N₂O₇ [M+H]⁺367, found 367.

Step 2: Under a nitrogen atmosphere, Pd/C (810.4 mg) was added to a solution of 1,5-dimethyl (2S)-2-(2-{[(benzyloxy)carbonyl]amino}acetamido)pentanedioate (3 g, 8.19 mmol, 1.0 eq.) in MeOH (50 mL). H₂ was subsequently introduced into the reaction system, and the resulting mixture was stirred at r.t. for 2 h. Upon completion, the reaction mixture was filtered and concentrated in vacuo to afford crude product which was used directly in the next step without further purification. MS: m/z: Calc'd for C₈H₁₂N₂O₄ [M+H⁺22]⁺223, found 223.

Step 3: To a stirred solution of methyl 3-[(2S)-3,6-dioxopiperazin-2-yl]propanoate (500 mg, 2.50 mmol, 1.0 eq.) in MeOH (10 mL) was added Ba(OH)₂ (1283.8 mg, 7.50 mmol, 3.0 eq.) at room temperature under nitrogen atmosphere. The solution was stirred at room temperature for 4 h. Desired product could be detected by LCMS. The crude product (1.6g) was purified by Prep-HPLC (0.05% TFA in water and acetonitrile) to afford 3-[(2S)-3,6-dioxopiperazin-2-yl]propanoic acid (80 mg, 17.21%) as a colorless oil. MS: m/z: Calc'd for C₇H₁₀N₂O₄[M+H]⁺187, found 187.

Step 4: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.24 mmol, 1.0 eq.) and 3-[(2S)-3,6-dioxopiperazin-2-yl]propanoic acid (65.9 mg, 0.35 mmol, 1.5 eq.) in DCM (5 mL) were added DCC (73.1 mg, 0.35 mmol, 1.5 eq.) and DMAP (28.9 mg, 0.24 mmol, 1.0 eq.) at 0° C. under nitrogen atmosphere. The resulting reaction mixture was stirred at ambient temperature for overnight. LCMS showed the reaction was complete. The resulting mixture was used in the next step directly without further purification. MS: m/z: Calc'd for C₂₉H₄₁N₃O₁₀ [M+H−100]⁺492, found 492.

Step 5: To a stirred mixture of tert-butyl (2S,3S,4S)-4-[(tert-butoxycarbonyl)oxy]-3-({3-[(2R)-3,6-dioxopiperazin-2-yl]propanoyl}oxy)-2-[(4-methoxyphenyl)methyl]pyrrolidine-1-carboxylate (100 mg, 0.17 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidine-3-yl 3-[(2S)-3,6-dioxopiperazin-2-yl]propanoate; trifluoroacetic acid (17.4 mg, 20.3%) as white solid. MS: m/z: Calc'd for C₁₉H₂₅N₃O₆ [M+H]⁺392, found 392. ¹H NMR (400 MHz, Methanol-d₄) δ 7.30-7.22 (m, 2H), 6.98-6.89 (m, 2H), 5.16-5.11 (m, 1H), 4.43-4.37 (m, 1H), 4.29 (dd, J=8.8, 4.5 Hz, 1H), 4.26-4.04 (m, 3H), 3.80 (s, 3H), 3.64 (dd, J=12.8, 4.5 Hz, 1H), 3.24 (d, J=12.8 Hz, 1H), 3.11 (dd, J=14.3, 7.0 Hz, 1H), 3.00 (dd, J=14.3, 8.6 Hz, 1H), 2.60-2.27 (m, 3H), 2.26-2.10 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 62 mL/min mL/min; Gradient: 2% B to 16% B in 10 min; Wave Length: 254 nm/220 nm nm; RT1(min): 8.68

### Synthetic Procedure of V000148

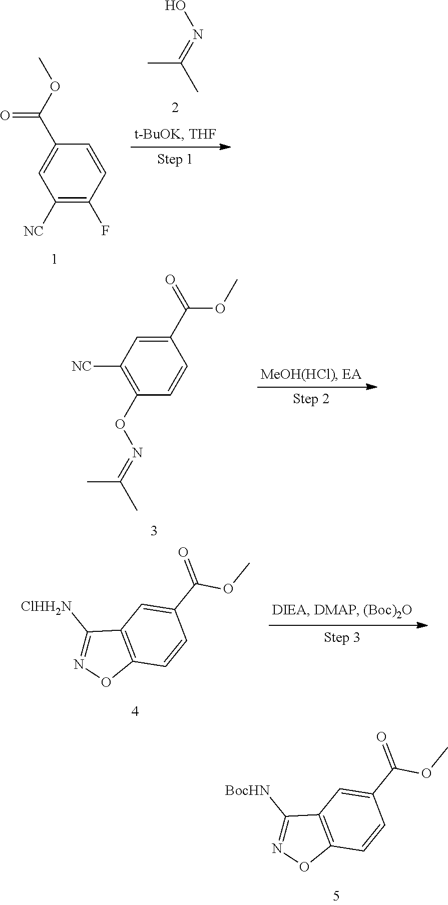

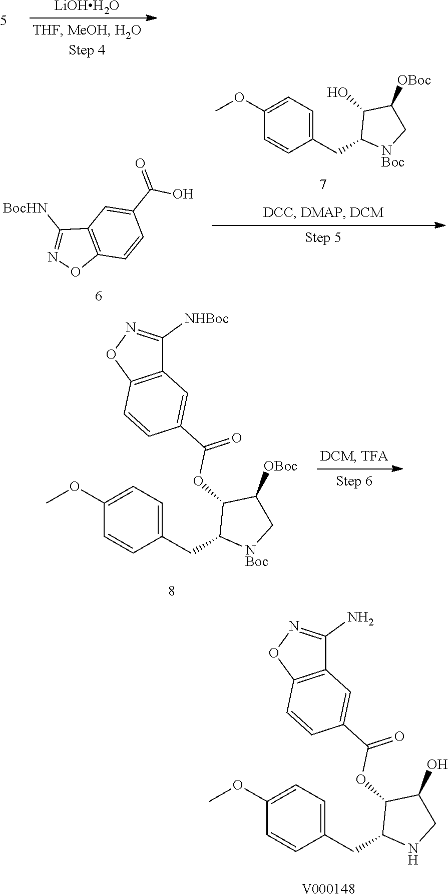

Step 1: To the solution of acetone oxime (448.81 mg, 6.140 mmol, 1.1 equiv) in THF (50 mL) was added t-BuOK (687.6 g, 6.14 mmol, 1.1 equiv), the mixture was stirred at room temperature for 0.5 h, then methyl 3-cyano-4-fluorobenzoate (1000 mg, 5.582 mmol, 1 equiv) was added, the mixture was stirred at room temperature overnight. The reaction was quenched by NH₄Cl solution. Then extracted by EA, washed with brine, dried, concentrated, then purified by silica gel column chromatography to afford methyl 3-cyano-4-[(propan-2-ylideneamino)oxy]benzoate (600 mg, 46.2%) as white solid.

Step 2: Methyl 3-cyano-4-[(propan-2-ylideneamino)oxy]benzoate (600 mg, 2.58 mmol, 1 equiv) was dissolved in HCl(g) MeOH (20 mL), the mixture solution was stirred at room temperature overnight. LCMS showed the reaction was completed, filtered, and the solid was get by filtration to afford methyl 3-amino-1,2-benzoxazole-5-carboxylate (500 mg, 100%).

Step 3: To the solution of methyl 3-amino-1,2-benzoxazole-5-carboxylate (500 mg, 2.60 mmol, 1 equiv) in pyridine (30 mL) was added (Boc)₂O (1703.5 mg, 7.80 mmol, 3.0 equiv), the mixture was stirred at 40° C. for overnight, then (Boc)₂O (1703.5 mg, 7.80 mmol, 3.0 equiv) was added and the mixture was stirred at 40° C. for another 18 h. Upon completion, the reaction mixture was concentrated in vacuo, and the crude was purified by silica gel column chromatography to afford methyl 3-[(tert-butoxycarbonyl)amino]-1,2-benzoxazole-5-carboxylate (200 mg, 26.3%) as white solid. MS: m/z: Calc'd for C₁₄H₁₆N₂O₅ [M+H−56]⁺237; Found, 237

Step 4: To the solution of methyl 3-[(tert-butoxycarbonyl)amino]-1,2-benzoxazole-5-carboxylate (700 mg, 2.40 mmol, 1 equiv) in THF (16 mL) and MeOH (4 mL) was added LiOH (172.0 mg, 7.18 mmol, 3.0 equiv) in H₂O (4 mL). The mixture was stirred at room temperature for overnight. Upon completion, the reaction mixture was concentrated in vacuo, then diluted with H₂O (10 mL), and the mixture was neutralized to Ph=7 with 1N HCl. The precipitated solids were collected by filtration and washed with water to afford 3-[(tert-butoxycarbonyl) amino]-1,2-benzoxazole-5-carboxylic acid (500 mg, 75.0%) as a white solid. MS: m/z: Calc'd for C₁₃H₁₄N₂O₅ [M−H]⁺277; Found, 277.

Step 5: To the mixture solution of 3-[(tert-butoxycarbonyl)amino]-1,2-benzoxazole-5-carboxylic acid (65.7 mg, 0.23 mmol, 2 equiv) in DCM (8 mL) were added DCC (36.5 mg, 0.17 mmol, 1.5 equiv), DMAP (17.3 mg, 0.14 mmol, 1.2 equiv), tert-butyl (2R,3S,4S)-4-[(tert-butoxycarbonyl)oxy]-3-hydroxy-2-[(4-methoxyphenyl)methyl]pyrrolidine-1-carboxylate (50 mg, 0.11 mmol, 1.00 equiv) and the mixture solution was stirred at room temperature overnight. LCMS showed the reaction was completed. The resulting mixture was filtered, the filtrate was concentrated under reduced pressure and then purified by silica gel column chromatography to afford (2R,3S,4S)-1-(tert-butoxycarbonyl)-4-[(tert-butoxycarbonyl)oxy]-2-[(4-methoxyphenyl)methyl]pyrrolidin-3-yl 3-[(tert-butoxycarbonyl)amino]-1,2-benzoxazole-5-carboxylate (80 mg, 99.1%) as colorless oil. MS: m/z: Calc'd for C₃₅H₄₅N₃O₁₁ [M+H]⁺684; Found, 684.

Step 6: To the solution of (2R,3S,4S)-1-(tert-butoxycarbonyl)-4-[(tert-butoxycarbonyl)oxy]-2-[(4-methoxyphenyl)methyl]pyrrolidin-3-yl 3-[(tert-butoxycarbonyl)amino]-1,2-benzoxazole-5-carboxylate (80 mg, 0.11 mmol, 1 equiv) in DCM (10 mL) was added TFA (2 mL), the mixture was stirred at room temperature for overnight. After completion of the reaction monitored by LCMS, the mixture was concentrated. The residue was purified by prep-HPLC to afford (2R,3S,4S)-4-hydroxy-2-[(4-methoxyphenyl)methyl]pyrrolidin-3-yl 3-amino-1,2-benzoxazole-5-carboxylate; trifluoroacetic acid as a white solid. (19.7 mg, yield 44.0%). MS: m/z: Calc'd for C₂₀H₂₁N₃O₅ [M+H]⁺384; Found, 384. ¹H NMR (400 MHz, DMSO-d₆) δ 8.72 (d, J=1.6 Hz, 1H), 8.35 (dd, J=8.8, 1.6 Hz, 1H), 7.66 (d, J=8.8 Hz, 1H), 7.23-7.15 (m, 2H), 6.94-6.84 (m, 2H), 5.16-5.12 (m, 1H), 4.43-4.37 (m, 1H), 4.18-4.18-4.11 (m, 1H), 3.72 (s, 3H), 3.68-3.57 (m, 1H), 3.19-2.99 (m, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 6% B to 36% B in 10 min; Wave Length: 254 nm/220 nm nm; RT1(min): 8.95

### Synthetic Procedure of V000173

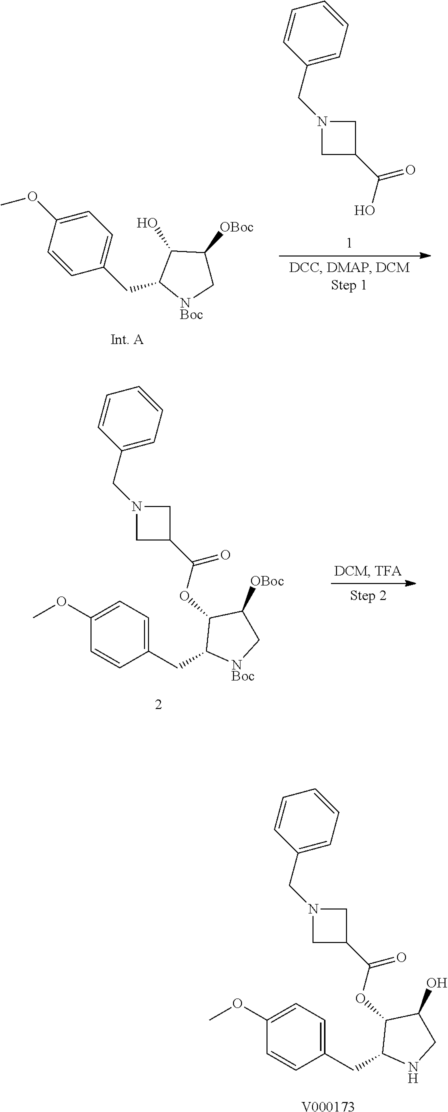

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 70 mg, 0.165 mmol, 1 eq.) and 1-benzylazetidine-3-carboxylic acid (37.93 mg, 0.198 mmol, 1.2 equiv) in DCM (5 mL) were added DCC (51.16 mg, 0.247 mmol, 1.5 eq.) and DMAP (20.19 mg, 0.165 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-3-(1-benzylazetidine-3-carbonyloxy)-4-[(tert-butoxycarbonyl)oxy]-2-[(4-methoxyphenyl)methyl]pyrrolidine-1-carboxylate (30 mg, 30.4%) as a white solid. MS: m/z: Calc'd for C₃₃H₄₄N₂O₈ [M+H]⁺597; Found 597.

Step 2: To a stirred mixture of tert-butyl (2R,3S,4S)-3-(1-benzylazetidine-3-carbonyloxy)-4-[(tert-butoxycarbonyl)oxy]-2-[(4-methoxyphenyl)methyl]pyrrolidine-1-carboxylate (30 mg, 0.05 mmol) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 1-benzylazetidine-3-carboxylate (21.3 mg, 85.8%) as a white solid. MS: m/z: Calc'd for C₂₃H₂₈N₂O₄ [M+H]⁺397; Found, 397. ¹H NMR (400 MHz, DMSO-d₆) δ 7.47 (s, 5H), 7.23 (d, J=8.1 Hz, 2H), 6.91 (d, J=8.2 Hz, 2H), 4.98-3.47 (m, 7H), 3.45 (d, J=12.6 Hz, 1H), 3.3 (d, J=12.6 Hz, 1H), 3.09-3.07 (m, 3H), 2.87 (t, J=11.5 Hz, 1H), 3.06-3.03 (m, 1H). Prep-HPLC conditions: Column: XSelect CSH Prep C18 OBD Column, 5 μm, 19*150 mm; Mobile Phase A: Water (0.05% TFA), Mobile Phase B:ACN; Flow rate:60 mL/min; Gradient:2 B to 25 B in 30 min; 254/220 nm; RT1:9.28; RT2:; Injection Volumn: ml; Number Of Runs.

### Synthetic Procedure of V000192

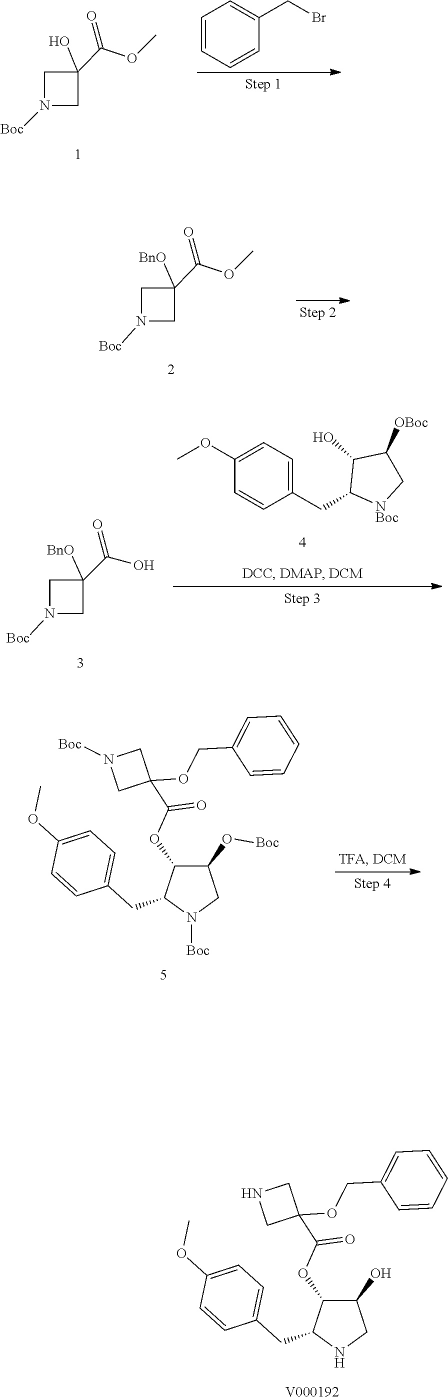

Step 1: To a stirred solution of 1-(tert-butyl) 3-methyl 3-hydroxyazetidine-1,3-dicarboxylate (1.5 g, 4.30 mmol, 1.0 eq.) in DMF (20 mL) was added NaH (198.2 mg, 4.70 mmol, 1.1 eq.) and BnBr (735.3 mg, 4.3 mmol, 1.0 eq.) sequentially at room temperature. The reaction was stirred at room temperature for 1 h. The reaction was poured into 50 mL ice water and extracted with EA (50 mL*3) and then washed by saturated NaCl. The organic phase was dried over Na₂SO₄. The solid was filtered and the filtrate was collected. The solvent was concentrated under reduced pressure to give 1-(tert-butyl) 3-methyl 3-(benzyloxy)azetidine-1,3-dicarboxylate (0.9 g, 65.1%) as a colorless oil. MS: m/z: Calc'd for C₁₇H₂₃NO₅ [M+H−100]⁺222, found 222.

Step 2: To a stirred solution of 1-(tert-butyl) 3-methyl 3-(benzyloxy)azetidine-1,3-dicarboxylate (550.0 mg, 1.72 mmol, 1.0 eq.) in 20.0 mL THF was added a solution of LiOH (49.1 mg, 2.05 mmol, 1.2 eq.) in 2.0 mL water at room temperature. The resulting mixture was stirred at room temperature for 6 h. The reaction was diluted with water (20.0 mL). The organic solvent was evaporated under vacuum by oil pump and then the pH of the aqueous phase was adjusted to 1 by adding 1N HCl. Extraction was performed with EA (30.0 mL*3), dried over Na₂SO₄, filtration and the filtrate was collected. Removing the solvent under reduced pressure gave 3-(benzyloxy)-1-(tert-butoxycarbonyl)azetidine-3-carboxylic acid (300.0 mg, 56.7% yield) as a colorless oil. MS: m/z: Calc'd for C₁₆H₂₁NO₅[M+H−56]⁺252, found 252.

Step 3: To a stirred solution of 3-(benzyloxy)-1-(tert-butoxycarbonyl)azetidine-3-carboxylic acid (100.0 mg, 0.33 mmol, 1.00 equiv) and tert-butyl (2R,3S,4S)-4-((tert-butoxycarbonyl) oxy)-3-hydroxy-2-(4-methoxybenzyl)pyrrolidine-1-carboxylate in 5.0 mL DCM were added DCC (67.1 mg, 0.33 mmol, 1.00 equiv) and DMAP (42.3 mg, 0.36 mmol, 1.10 equiv) sequentially at room temperature. The resulting solution was stirred at room temperature for 6 h. the solvent was removed under reduced pressure and the residue was purified by reversed-phase column chromatography ((Spherial C18, 80 g, 20-40 μm; Mobile Phase A: water (0.05% TFA), Mobile Phase B: ACN; Flow rate: 70 mL/min; Gradient: 0% B to 70% B in 20 min, 210 nm; RT: 14.2 min)). The solvent was removed under reduced to give 3-((2R,3S,4S)-1-(tert-butoxycarbonyl)-4-((tert-butoxycarbonyl)oxy)-2-(4-methoxybenzyl) pyrrolidin-3-yl) 1-(tert-butyl) 3-(benzyloxy)azetidine-1,3-dicarboxylate (60.0 mg, 25.5% yield) as a colorless oil. MS: m/z: Calc'd for C₃₈H₅₂N₂O₁₁ [M+H−56−56−100]⁺501, found 501.

Step 4: To a stirred mixture of 3-((2R,3S,4S)-1-(tert-butoxycarbonyl)-4-((tert-butoxycarbonyl)oxy)-2-(4-methoxybenzyl)pyrrolidin-3-yl) 1-(tert-butyl) 3-(benzyloxy)azetidine-1,3-dicarboxylate (60.0 mg, 0.08 mmol) in DCM(3 mL) was added TFA (3.0 mL). The solution was stirred at room temperature for 3 h. The solvent was removed under reduced pressure. The residue was purified by Prep-HPLC to afford (2R,3S,4S)-4-hydroxy-2-(4-methoxybenzyl) pyrrolidin-3-yl 3-(benzyloxy)azetidine-3-carboxylate TFA salt (20.0 mg, 37.2% yield) as a white solid. Calc'd for C₂₃H₂₈N₂O₅ [M+H]⁺413; Found, 413. ¹H NMR (400 MHz, DMSO-d₆) δ 9.50-9.95 (br, 4H), 7.40 m, 5H), 7.26 (dd, J=8.7, 2.9 Hz, 2H), 6.91 (dd, J=8.6, 3.0 Hz, 2H), 6.16 (d, J=4.2 Hz, 1H), 5.13 (s, 1H), 4.62 (d, J=2.9 Hz, 2H), 4.52 (d, J=13.6 Hz, 1H), 4.45 (s, 1H), 4.22 (d, J=11.7 Hz, 2H), 4.14 (s, 1H), 3.74 (d, J=3.0 Hz, 3H), 3.58 (d, J=12.3 Hz, 1H), 3.15-3.04 (m, 2H), 2.93 (t, J=11.9 Hz, 1H), 1.24 (s, 1H). Prep-HPLC (Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm nm; RT1(min): 8.65.

### Synthetic Procedure of V000192

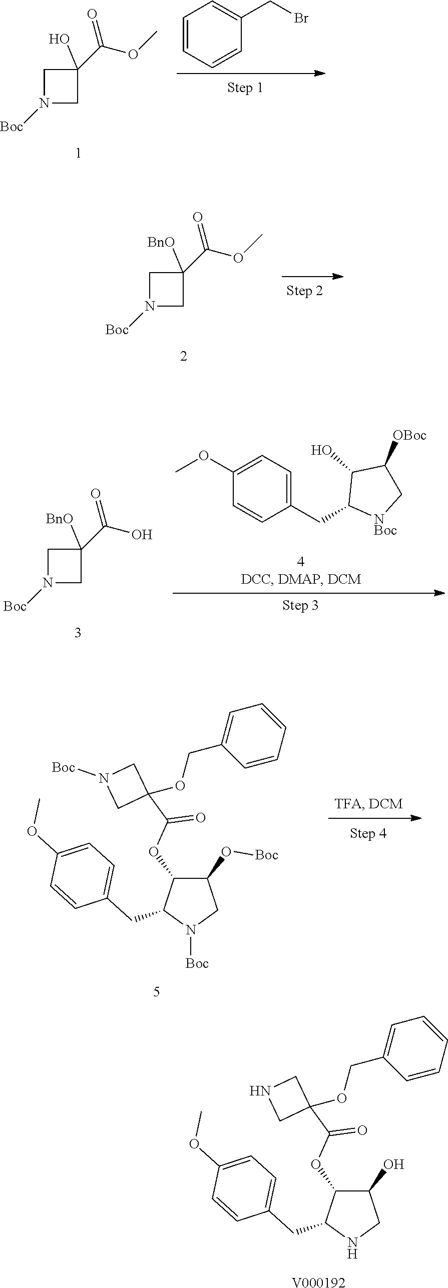

Step 1: To a stirred solution of 1-(tert-butyl) 3-methyl 3-hydroxyazetidine-1,3-dicarboxylate (1.5 g, 4.30 mmol, 1.00 equiv) in 20.0 mL DMF was added NaH (198.2 mg, 4.70 mmol, 1.10 equiv) and BnBr (735.3 mg, 4.30 mmol, 1.00 equiv) sequentially at room temperature. The reaction was stirred at room temperature for 1 h. The reaction was poured into 50 mL ice water and extracted with EA (50 mL*3) and then washed by saturated NaCl. The organic phase was dried over Na₂SO₄. The solid was filtered and the filtrate was collected. The solvent was concentrated under reduced pressure to give 1-(tert-butyl) 3-methyl 3-(benzyloxy)azetidine-1,3-dicarboxylate (0.9 g, 65.1%) as a colorless oil. MS: m/z: Calc'd for C₁₇H₂₃NO₅ [M+H]⁺322, found 322.

Step 2: To a stirred mixture of 1-(tert-butyl) 3-methyl 3-(benzyloxy)azetidine-1,3-dicarboxylate (550.0 mg, 1.72 mmol, 1.00 equiv) in 20.0 mL THF was added a solution of LiOH (49.1 mg, 2.05 mmol, 1.20 equiv) in 2.0 mL water at room temperature. The resulting mixture was stirred at room temperature for 6 h. The reaction was diluted with 20.0 mL water. The organic solvent was evaporated under vacuum by oil pump and then the pH of aqueous phase was adjusted to 1 by adding 1N HCl, extracted with 30.0 mL*3 EA, dried over Na₂SO₄, filtered and the filtrate was removed under reduced pressure to give 3-(benzyloxy)-1-(tert-butoxycarbonyl)azetidine-3-carboxylic acid (300.0 mg, 56.7% yield) as a colorless oil. MS: m/z: Calc'd for C₁₆H₂₁NO₅ [M+H]⁺308, found 308.

Step 3: To a stirred solution of 3-(benzyloxy)-1-(tert-butoxycarbonyl)azetidine-3-carboxylic acid (100.0 mg, 0.33 mmol, 1.00 equiv) and tert-butyl (2R,3S,4S)-4-((tert-butoxycarbonyl) oxy)-3-hydroxy-2-(4-methoxybenzyl)pyrrolidine-1-carboxylate in 5.0 mL DCM were added DCC (67.1 mg, 0.33 mmol, 1.00 equiv) and DMAP (42.3 mg, 0.36 mmol, 1.10 equiv) sequentially at room temperature. The resulting solution was stirred at room temperature for 6 h. the solvent was removed under reduced pressure and the residue was purified by reversed-phase column chromatography ((Spherial C18, 80 g, 20-40 μm; Mobile Phase A: water (0.05% TFA), Mobile Phase B: ACN; Flow rate: 70 mL/min; Gradient: 0% B to 70% B in 20 min, 210 nm; RT: 14.2 min)) to afford 3-((2R,3S,4S)-1-(tert-butoxycarbonyl)-4-((tert-butoxycarbonyl) oxy)-2-(4-methoxybenzyl)pyrrolidin-3-yl) 1-(tert-butyl) 3-(benzyloxy)azetidine-1,3-dicarboxylate (60.0 mg, 25.5% yield) as a colorless oil. MS: m/z: Calc'd for C₃₈H₅₂N₂O₁₁ [M+H]⁺713, found 713.

Step 4: To a stirred mixture of 3-((2R,3S,4S)-1-(tert-butoxycarbonyl)-4-((tert-butoxy carbonyl)oxy)-2-(4-methoxybenzyl)pyrrolidin-3-yl) 1-(tert-butyl) 3-(benzyloxy)azetidine-1,3-dicarboxylate (60.0 mg, 0.08 mmol, 1.00 equiv) in DCM was added 3.0 mL TFA. The solution was stirred at room temperature for 3 h. The solvent was removed under reduced pressure and then purified by Prep-HPLC to afford (2R,3S,4S)-4-hydroxy-2-(4-methoxybenzyl) pyrrolidin-3-yl 3-(benzyloxy)azetidine-3-carboxylate (20.0 mg, 37.2% yield) as a white solid. Calc'd for C₂₃H₂₈N₂O₅ [M+H]⁺413; Found, 413. ¹H NMR (400 MHz, DMSO-d₆) δ 9.50-9.95 (br, 4H), 7.40 m, 5H), 7.26 (dd, J=8.7, 2.9 Hz, 2H), 6.91 (dd, J=8.6, 3.0 Hz, 2H), 6.16 (d, J=4.2 Hz, 1H), 5.13 (s, 1H), 4.62 (d, J=2.9 Hz, 2H), 4.52 (d, J=13.6 Hz, 1H), 4.45 (s, 1H), 4.22 (d, J=11.7 Hz, 2H), 4.14 (s, 1H), 3.74 (d, J=3.0 Hz, 3H), 3.58 (d, J=12.3 Hz, 1H), 3.15-3.04 (m, 2H), 2.93 (t, J=11.9 Hz, 1H), 1.24 (s, 1H). Prep-HPLC (Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm nm; RT1(min): 8.65.

### Synthetic Procedure of V000196

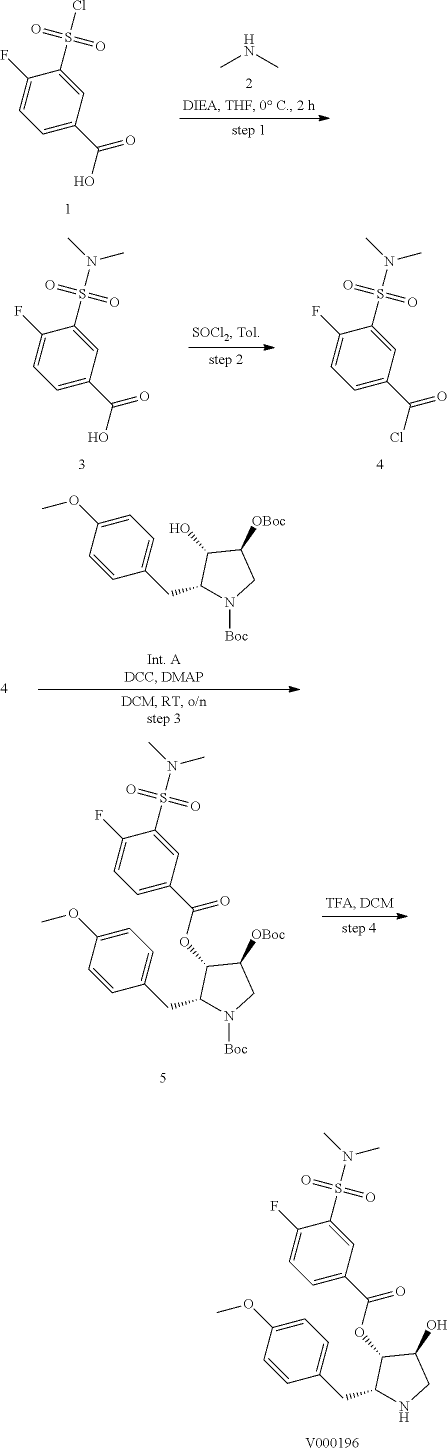

Step 1: To a stirred solution of 3-(chlorosulfonyl)-4-fluorobenzoic acid (500 mg, 2.10 mmol, 1.0 eq.) and dimethylamine (283.4 mg, 6.29 mmol, 3.0 eq.) in THF (5 mL) was added DIEA (812.5 mg, 6.29 mmol, 3.0 eq.) at 0° C. and the resulting mixture was stirred 2 h under a nitrogen atmosphere. Upon completion, the resulting mixture was concentrated under reduced pressure to afford crude product 3-(dimethylsulfamoyl)-4-fluorobenzoic acid (500 mg, 96.51%) as a light yellow oil, which was used directly in the next step without further purification. MS: m/z: Calc'd for C₉H₁₀FNO₄S [M−H]⁻246, found 246.

Step 2: To a stirred solution of 3-(dimethylsulfamoyl)-4-fluorobenzoic acid (480 mg, 1.94 mmol, 1.0 eq.) in Toluene (5 mL) was added SOCl₂ (1154.8 mg, 9.71 mmol, 5.0 eq.) at room temperature. The resulting mixture was stirred at 80° C. for 1 h under a nitrogen atmosphere. Upon completion, the resulting mixture was concentrated under reduced pressure. The crude product was used in the next step directly without further purification.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and 3-(dimethylsulfamoyl)-4-fluorobenzoyl chloride (480 mg, 1.81 mmol, 9.6 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-[3-(dimethylsulfamoyl)-4-fluorobenzoyloxy]-2-[(4-methoxyphenyl)methyl]pyrrolidine-1-carboxylate (90 mg, 73.0%) as a light yellow oil. MS: m/z: Calc'd for C₃₁H₄₁FN₂O₁₀S [M+H⁺22]⁺675, found 675.

Step 4: To a stirred mixture of tert-butyl (2R,3S,4S)-4-[(tert-butoxycarbonyl)oxy]-3-[3-(dimethylsulfamoyl)-4-fluorobenzoyloxy]-2-[(4-methoxyphenyl)methyl]pyrrolidine-1-carboxylate (60 mg, 0.09 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(dimethylsulfamoyl)-4-fluorobenzoate; trifluoroacetic acid (29.5 mg, 56.3%) as a white solid. MS: m/z: Calc'd for C₂₁H₂₅FN₂O₆S [M+H]⁺453, found 453. ¹H NMR (400 MHz, DMSO-d₆) δ 9.63 (s, 2H), 9.53 (s, 2H), 8.56-8.52 (m, 3H), 8.38 (dd, J=6.6, 2.3 Hz, 3H), 7.82-7.73 (m, 3H), 7.22-7.14 (m, 6H), 6.93-6.85 (m, 6H), 6.16 (s, 1H), 5.13 (d, J=3.2 Hz, 3H), 4.41 (d, J=4.4 Hz, 3H), 4.17-4.13 (m, 3H), 3.72 (s, 10H), 3.64-3.55 (m, 5H), 3.15-2.97 (m, 9H), 2.79 (d, J=1.8 Hz, 16H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 11% B to 41% B in 10 min; Wave Length: 254 nm/220 nm nm; RT1(min): 9.13

### Synthetic Procedure of V000198

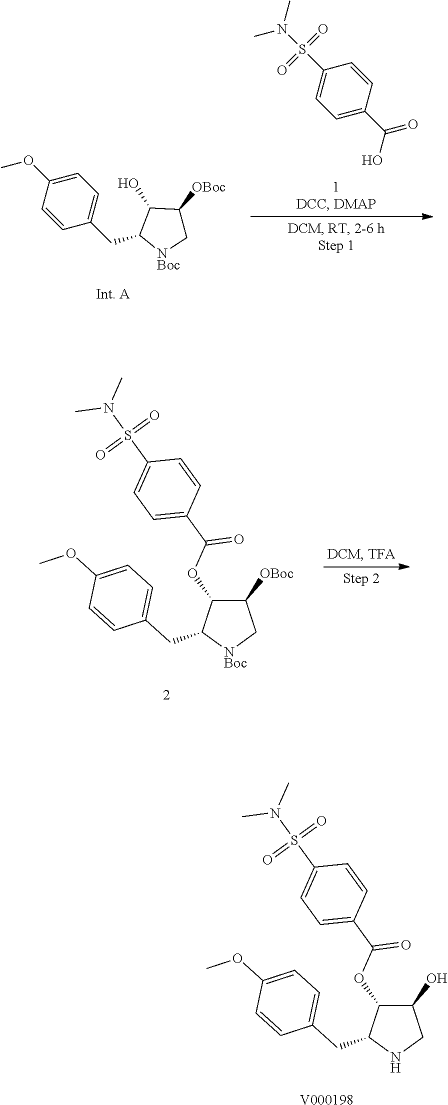

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 4-(dimethylsulfamoyl)benzoic acid (108.26 mg, 0.472 mmol, 2 eq.) in DCM (5 mL) were added DCC (73.1 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-[4-(dimethylsulfamoyl)benzoyloxy]-2-[(4-methoxyphenyl)methyl]pyrrolidine-1-carboxylate (110 mg, 73.3%) as a white solid. MS: m/z: Calc'd for C₃₁H₄₂N₂O₁₀S [M+H]⁺635; found 635.

Step 2: To a stirred mixture of tert-butyl (2R,3S,4S)-4-[(tert-butoxycarbonyl)oxy]-3-[4-(dimethylsulfamoyl)benzoyloxy]-2-[(4-methoxyphenyl)methyl]pyrrolidine-1-carboxylate (100 mg, 0.158 mmol, 1 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to a (2R,3S,4S)-4-hydroxy-2-[(4-methoxyphenyl)methyl]pyrrolidin-3-yl 4-(dimethylsulfamoyl)benzoate; trifluoroacetic acid (27.7 mg, 31.9%) as a white solid. MS: m/z: Calc'd for C₂₁H₂₆N₂O₆S [M+H]⁺435; Found 435. ¹H NMR (400 MHz, DMSO-d₆) δ 9.63 (s, 2H), 8.41-8.34 (m, 2H), 7.99-7.91 (m, 2H), 7.20 (d, J=8.4 Hz, 2H), 6.93-6.86 (m, 2H), 6.14 (s, 1H), 5.16 (d, J=3.1 Hz, 1H), 4.40 (d, J=4.3 Hz, 1H), 4.14 (s, 1H), 3.73 (s, 2H), 3.38 (s, 2H), 3.21-3.08 (m, 3H), 2.67 (s, 6H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 11% B to 41% B in 10 min; Wave Length: 254 nm/220 nm nm; RT1(min): 8.63

### Synthetic Procedure of V000215

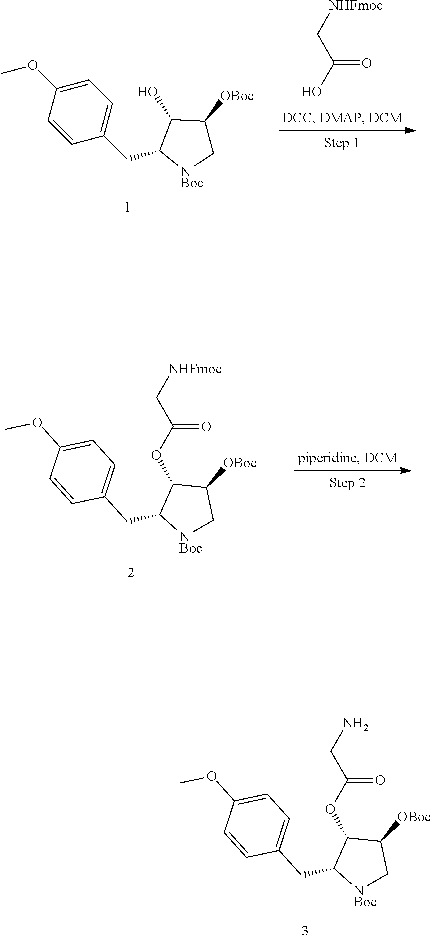

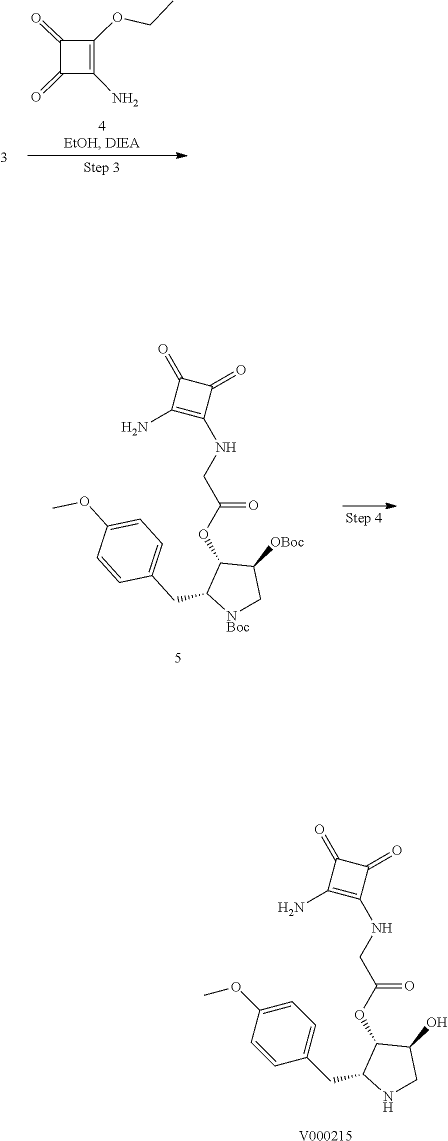

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int A, 150 mg, 0.35 mmol, 1 eq.) and {[(9H-fluoren-9-ylmethoxy)carbonyl]amino}acetic acid (105.3 mg, 0.35 mmol, 1 eq.) in DCM (8 mL) were added DCC (146.2 mg, 0.71 mmol, 2 eq.) and DMAP (64.9 mg, 0.53 mmol, 1.5 eq.) at 0° C. under nitrogen atmosphere. The mixture was stirred at room temperature for overnight. Then the reaction was concentrated, the crude was dissolved with DMSO and purified by reversed-phase column to obtain tert-butyl (2R,3S,4S)-4-[(tert-butoxycarbonyl)oxy]-3-[(2-{[(9H-fluoren-9-ylmethoxy)carbonyl]amino}acetyl)oxy]-2-[(4-methoxyphenyl)methyl]pyrrolidine-1-carboxylate (220 mg, 88.4%) as a white solid. MS: m/z: Calc'd for C₃₉H₄₆N₂O₁₀ [M+H−56−56]⁺591, found 591.

Step 2: To a stirred solution of tert-butyl (2R,3S,4S)-4-[(tert-butoxycarbonyl)oxy]-3-[(2-{[(9H-fluoren-9-ylmethoxy)carbonyl]amino}acetyl)oxy]-2-[(4-methoxyphenyl)methyl]pyrrolidine-1-carboxylate (215 mg, 0.31 mmol, 1 eq.) in DCM (6 mL) was added piperidine (1 mL) at room temperature for 1 h. The reaction was concentrated, the crude was dissolved with DMSO and purified by reverse-phase column to obtain tert-butyl (2R,3S,4S)-3-[(2-aminoacetyl) oxy]-4-[(tert-butoxycarbonyl)oxy]-2-[(4-methoxyphenyl)methyl]pyrrolidine-1-carboxylate (140 mg, 95.2%) as a white solid. MS: m/z: Calc'd for C₂₄H₃₆N₂O₈[M+H]⁺481, found 481.

Step 3: To a stirred solution of tert-butyl (2R,3S,4S)-3-[(2-aminoacetyl)oxy]-4-[(tert-butoxycarbonyl)oxy]-2-[(4-methoxyphenyl)methyl]pyrrolidine-1-carboxylate (120 mg, 0.25 mmol, 1 eq.) and 3-amino-4-ethoxycyclobut-3-ene-1,2-dione (211.4 mg, 1.5 mmol, 6 eq.) in EtOH (7 mL) was added DIEA (96.8 mg, 0.75 mmol, 3 eq.) at room temperature and the resulting mixture was stirred for overnight. The solvent was removed and the residue was purified by reversed-phase column to obtain tert-butyl (2R,3S,4S)-3-({2-[(2-amino-3,4-dioxocyclobut-1-en-1-yl)amino]acetyl}oxy)-4-[(tert-butoxycarbonyl)oxy]-2-[(4-methoxyphenyl)methyl]pyrrolidine-1-carboxylate (70 mg, 48.7%) as a yellow solid. MS: m/z: Calc'd for C₂₈H₃₇N₃O₁₀ [M+H−100−56]⁺420, found 420.

Step 4: To a stirred solution of tert-butyl (2R,3S,4S)-3-({2-[(2-amino-3,4-dioxocyclobut-1-en-1-yl)amino]acetyl}oxy)-4-[(tert-butoxycarbonyl)oxy]-2-[(4-methoxyphenyl)methyl]pyrrolidine-1-carboxylate (60 mg, 0.11 mmol, 1 eq.) in DCM (5 mL) was added TFA (1 mL) and the resulting mixture was stirred at room temperature for 2 h. After completion of the reaction monitored by LCMS, the mixture was concentrated. The residue was purified Prep-HPLC to obtain (2R,3S,4S)-4-hydroxy-2-[(4-methoxyphenyl)methyl]pyrrolidin-3-yl 2-[(2-amino-3,4-dioxocyclobut-1-en-1-yl)amino]acetate; trifluoroacetic acid (23.6 mg, 45.4%) as a white solid. MS: m/z: Calc'd for C₁₈H₂₁N₃O₆ [M+H]⁺376; found 376. ¹H NMR (400 MHz, DMSO-d₆) δ 7.20 (d, J=8.1 Hz, 2H), 6.87 (d, J=8.1 Hz, 2H), 4.90 (d, J=3.4 Hz, 1H), 4.51 (s, 1H), 4.42 (s, 1H), 4.24 (d, J=4.3 Hz, 1H), 4.02 (d, J=6.9 Hz, 1H), 3.70 (s, 3H), 3.45 (dd, J=13.1, 4.5 Hz, 1H), 3.09 (d, J=12.8 Hz, 1H), 2.93 (d, J=7.8 Hz, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 74 mL/min mL/min; Gradient: 39% B to 69% B in 10 min; Wave Length: 254 nm/220 nm nm; RT1(min): 8.68

### Synthetic Procedure of V041828

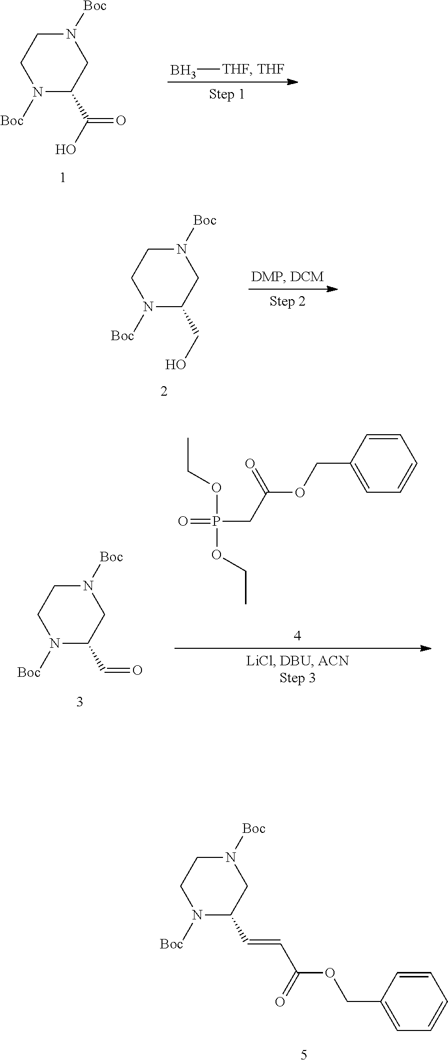

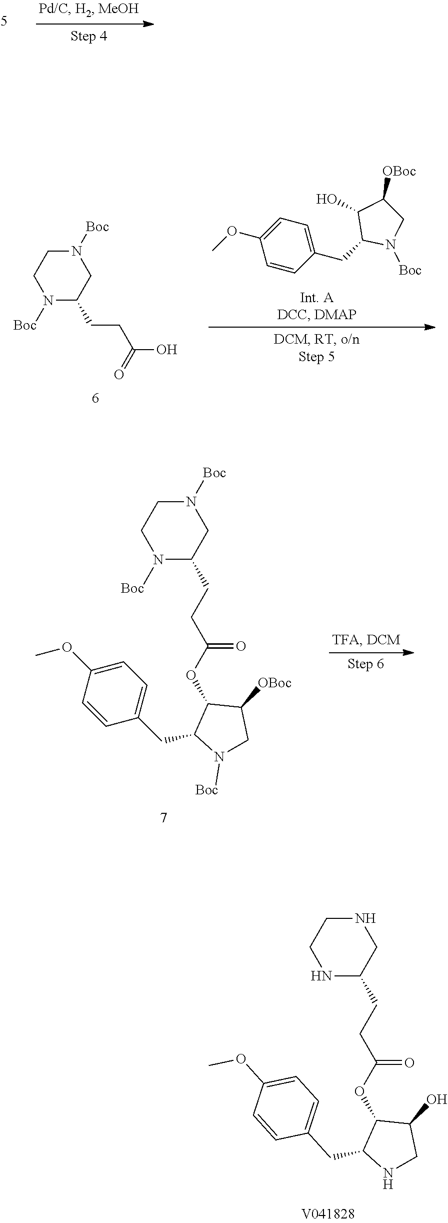

Step 1: To a stirred solution of (2R)-1,4-bis(tert-butoxycarbonyl)piperazine-2-carboxylic acid (1 g, 3.03 mmol, 1 eq.) in THF (20 mL) was added BH₃-THF (2.6 g, 30.27 mmol, 10 eq.) at 0° C. The resulting reaction mixture was stirred at 50° C. for overnight under a nitrogen atmosphere. The reaction was quenched with Water at 0° C. The aqueous layer was extracted with EtOAc (3×1 mL). The resulting mixture was concentrated under reduced pressure. This resulted in 1,4-di-tert-butyl (2R)-2-(hydroxymethyl)piperazine-1,4-dicarboxylate (700 mg, 73.1%) as a light yellow oil. MS: m/z: Calc'd for C₁₅H₂₈N₂O₅ [M+H−56−100]⁺161, found 161.

Step 2: To a stirred solution of 1,4-di-tert-butyl (2R)-2-(hydroxymethyl)piperazine-1,4-dicarboxylate (650 mg, 2.05 mmol, 1.0 eq.) in DCM (10 mL) was added DMP (1742.70 mg, 4.11 mmol, 2 eq.) at room temperature. The resulting reaction mixture was stirred at ambient temperature for 1 h. The resulting mixture was filtered, the filter cake was washed with DCM (3×1 mL). The filtrate was concentrated under reduced pressure to afford crude product 1,4-di-tert-butyl (2R)-2-formylpiperazine-1,4-dicarboxylate (400 mg, 61.9%) as a colorless oil. ¹H NMR (400 MHz, Chloroform-d) δ 9.61 (s, 1H), 4.71-4.25 (m, 2H), 3.86 (d, J=35.3 Hz, 2H), 3.25-3.04 (m, 2H), 2.92 (s, 1H), 1.48 (d, J=12.4 Hz, 18H).

Step 3: To a stirred solution of benzyl 2-(diethoxyphosphoryl)acetate (473.5 mg, 1.65 mmol, 1.3 eq.) in ACN (1 mL) was added DBU (232.4 mg, 1.53 mmol, 1.2 eq.) and LiCl (75.5 mg, 1.78 mmol, 1.4 eq.) at 0° C. and the mixture was stirred at the same temperature for 0.5 h. Then 1,4-di-tert-butyl (2R)-2-formylpiperazine-1,4-dicarboxylate (400 mg, 1.27 mmol, 1.0 eq.) was added at 0° C. The resulting reaction mixture was stirred at ambient temperature for 2 h. Desired product could be detected by LCMS. The resulting mixture was filtered, the filter cake was washed with MeCN (2×1 mL). The filtrate was concentrated under reduced pressure. The residue was purified by Prep-TLC (PE/EA 1:1) to afford 1,4-di-tert-butyl (2S)-2-[3-(benzyloxy)-3-oxoprop-1-en-1-yl]piperazine-1,4-dicarboxylate (520 mg, 91.5%) as a light yellow oil. MS: m/z: Calc'd for C₂₄H₃₄N₂O₆ [M+H−56−100]⁺291, found 291.

Step 4: Under a nitrogen atmosphere, Pd/C (100 mg) was added to a solution of 1,4-di-tert-butyl (2S)-2-[3-(benzyloxy)-3-oxoprop-1-en-1-yl]piperazine-1,4-dicarboxylate (200 mg, 0.45 mmol, 1.0 eq.) in MeOH (5 mL), H₂ was subsequently introduced into the reaction system, and the resulting mixture was stirred at r.t. for 1 h. Upon completion, the reaction mixture was filtered and concentrated in vacuo to afford crude product 3-[(2S)-1,4-bis(tert-butoxycarbonyl) piperazin-2-yl]propanoic acid (160 mg, 99.7%) as a light yellow oil. MS: m/z: Calc'd for C₁₇H₃₀N₂O₆ [M+H−56−100]⁺203, found 203.

Step 5: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 90 mg, 0.21 mmol, 1.0 eq.) and 3-[(2S)-1,4-bis(tert-butoxycarbonyl)piperazin-2-yl]propanoic acid (152.3 mg, 0.43 mmol, 2.0 eq.) in DCM (5 mL) were added DCC (65.8 mg, 0.32 mmol, 1.5 eq.) and DMAP (26.0 mg, 0.21 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved in DMSO and purified by a reversed-phase column chromatography (0.05% TFA in water and acetonitrile) to afford 1,4-di-tert-butyl (2S)-2-(3-{[(2R,3S,4S)-1-(tert-butoxycarbonyl)-4-[(tert-butoxycarbonyl)oxy]-2-[(4-methoxyphenyl) methyl]pyrrolidin-3-yl]oxy}-3-oxopropyl)piperazine-1,4-dicarboxylate (120 mg, 73.9%) as a light yellow oil. MS: m/z: Calc'd for C₃₉H₆₁N₃O₁₂ [M+H−56−56−100-100]⁺452, found 452.

Step 6: To a stirred mixture of 1,4-di-tert-butyl (2S)-2-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)piperazine-1,4-dicarboxylate (115 mg, 0.15 mmol, 1.0 eq.) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-[(2S)-piperazin-2-yl]propanoate; trifluoroacetic acid (25.2 mg, 33.5%) as a white solid. MS: m/z: Calc'd for C₁₉H₂₉N₃O₄ [M+H]⁺364, found 364. ¹H NMR (400 MHz, Methanol-d₄) δ 7.25 (d, J=8.3 Hz, 2H), 6.94 (dd, J=8.7, 2.5 Hz, 2H), 5.15 (d, J=3.0 Hz, 1H), 4.42 (d, J=4.5 Hz, 1H), 4.21 (d, J=9.2 Hz, 1H), 3.80 (d, J=2.3 Hz, 3H), 3.71-3.53 (m, 5H), 3.21-2.73 (m, 6H), 2.16-1.99 (m, 2H). Prep-HPLC-conditions: Column: Atlantis T3 Prep OBD Column, 19*250 mm, 5 μm; Mobile Phase A: Water (0.1% TFA), Mobile Phase B: ACN; Flow rate: 25 mL/min mL/min; Gradient: 2% B to 24% B in 8 min; Wave Length: 254 nm/220 nm nm; RT1(min): 7.42

### Synthetic Procedure of V186712

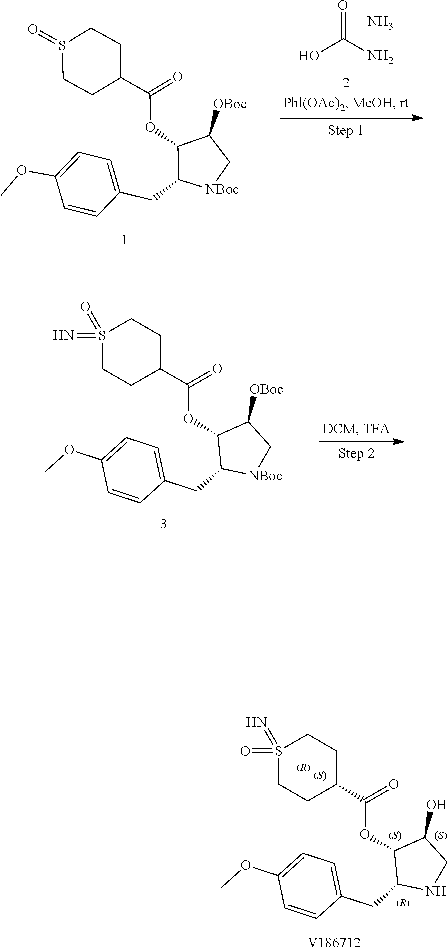

Step 1: To a solution of tert-butyl (2R,3S,4S)-4-[(tert-butoxycarbonyl)oxy]-2-[(4-methoxy phenyl)methyl]-3-(1-oxo-1lambda4-thiane-4-carbonyloxy)pyrrolidine-1-carboxylate (160 mg, 0.28 mmol, 1 eq.), in MeOH (5 mL) were added ammonium carbamate (165.0 mg, 2.11 mmol, 7.5 eq.) and DIB (408.5 mg, 1.26 mmol, 4.5 eq.) at room temperature. The reaction mixture was stirred at room temperature for 2 h. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-(1-imino-1-oxo-1lambda6-thiane-4-carbonyloxy)-2-[(4-methoxyphenyl) methyl]pyrrolidine-1-carboxylate (140 mg, 85.2%) as a white solid. MS: m/z: Calc'd for C₂₈H₄₂N₂O₉S [M+H]⁺583; Found 583.

Step 2: To a stirred mixture of tert-butyl (2R,3S,4S)-4-[(tert-butoxycarbonyl)oxy]-3-(1-imino-1-oxo-1lambda6-thiane-4-carbonyloxy)-2-[(4-methoxyphenyl)methyl]pyrrolidine-1-carboxylate (135 mg, 0.23 mmol) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl (1 s)-1-imino-1-oxo-1lambda6-thiane-4-carboxylate; trifluoroacetic acid salt (16.2 mg, 14.0%) as a white solid. MS: m/z: Calc'd for C₁₈H₂₆N₂O₅S [M+H]⁺383; Found 383. ¹H NMR (400 MHz, DMSO-d₆) δ 9.77 (s, 1H), 9.56 (s, 1H), 7.20 (d, J=8.1 Hz, 2H), 6.90 (d, J=8.2 Hz, 2H), 4.90 (d, J=3.4 Hz, 1H), 4.25 (d, J=4.3 Hz, 1H), 4.03 (s, 1H), 3.77 (d, J=13.4 Hz, 3H), 3.73 (s, 3H), 3.70 (d, J=8.3 Hz, 1H), 3.67 (d, J=10.7 Hz, 1H), 3.08 (d, J=12.6 Hz, 1H), 2.96-2.85 (m, 3H), 2.46 (d, J=10.1 Hz, 1H), 2.21 (s, 1H), 2.16-2.09 (m, 1H), 2.07 (s, 1H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 6% B in 10 min; Wave Length: 254 nm/220 nm nm; RT1(min): 8.5

### Synthetic Procedure of V186717 and V186718

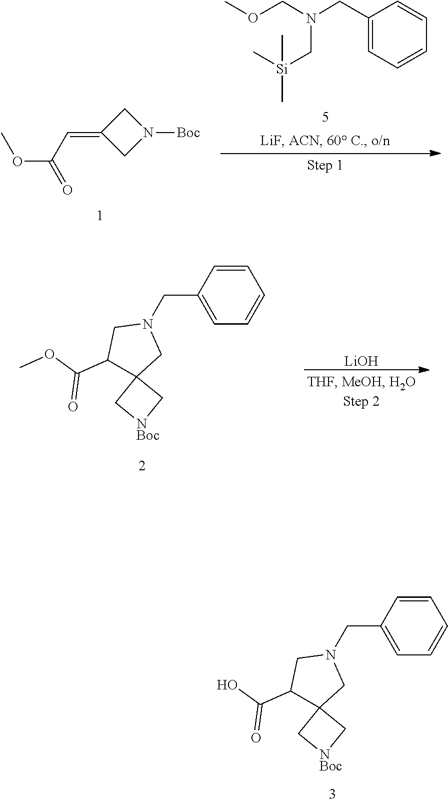

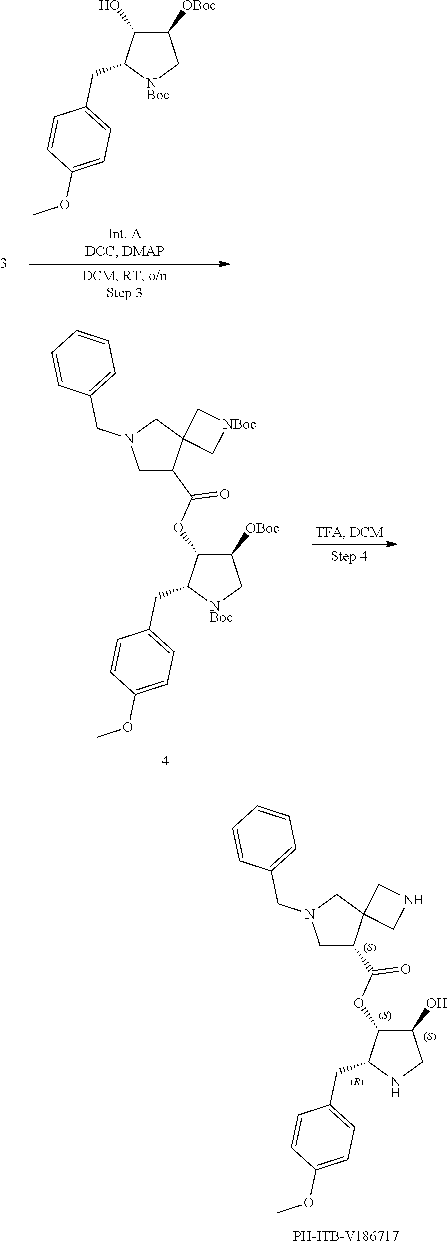

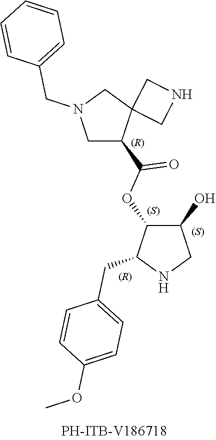

Step 1: To a stirred solution of tert-butyl 3-(2-methoxy-2-oxoethylidene)azetidine-1-carboxylate (2 g, 8.80 mmol, 1.0 eq.) and benzyl(methoxy)[(trimethylsilyl)methyl]amine (1.97 g, 8.800 mmol, 1 equiv) in ACN (20 mL) was added LiF (910 mg, 35.20 mmol, 4.0 eq.) in portions at room temperature. The resulting mixture was stirred at 60° C. for 2 h under a nitrogen atmosphere. Upon completion, the resulting mixture was filtered, the filter cake was washed with MeCN (3×10 mL). The filtrate was concentrated under reduced pressure. The residue was purified by reversed-phase flash chromatography (0.05% TFA in water and acetonitrile) to afford 2-tert-butyl 8-methyl 6-benzyl-2,6-diazaspiro[3.4]octane-2,8-dicarboxylate (2.3 g, 72.5%) as a light yellow oil. MS: m/z: Calc'd for C₂₀H₂₈N₂O₄ [M+H]⁺361, found 361.

Step 2: To a stirred solution of 2-tert-butyl 8-methyl 6-benzyl-2,6-diazaspiro[3.4]octane-2,8-dicarboxylate (500 mg, 1.387 mmol, 1 eq.) in THF (5 mL), MeOH (2.5 mL) and H₂O (2.5 mL) was added LiOH (99.7 mg, 4.16 mmol, 3.0 eq.) in portions at room temperature. The resulting mixture was stirred at 50° C. for 1 h. Upon completion, the resulting mixture was extracted with EtOAc (3×10 mL. The combined organic layers were washed with water (3×10 mL, dried over anhydrous Na₂SO₄. After filtration, the filtrate was concentrated under reduced pressure. The residue was purified by reversed-phase flash chromatography (0.05% TFA in water and acetonitrile) to afford 6-benzyl-2-(tert-butoxycarbonyl)-2,6-diazaspiro[3.4]octane-8-carboxylic acid (400 mg, 83.2%) as a light yellow oil. MS: m/z: Calc'd for C₁₉H₂₆N₂O₄ [M+H]⁺347, found 347.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.29 mmol, 1.0 eq.) and tert-butyl (2R,3S,4S)-4-[(tert-butoxycarbonyl)oxy]-3-hydroxy-2-[(4-methoxyphenyl) methyl]pyrrolidine-1-carboxylate (244.5 mg, 0.58 mmol, 2.0 eq.) in DCM (5 mL) were added DCC (89.3 mg, 0.43 mmol, 1.5 eq.) and DMAP (35.3 mg, 0.29 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford 2-tert-butyl 8-(2R,3S,4S)-4-hydroxy-2-[(4-methoxyphenyl)methyl]pyrrolidin-3-yl 6-benzyl-2,6-diazaspiro[3.4]octane-2,8-dicarboxylate (90 mg, 56.5%) as a light yellow oil. MS: m/z: Calc'd for C₄₁H₅₇N₃O₁₀ [M+H]⁺752, found 752.

Step 4: To a stirred mixture of 2-tert-butyl 8-(2R,3S,4S)-4-hydroxy-2-[(4-methoxyphenyl) methyl]pyrrolidin-3-yl 6-benzyl-2,6-diazaspiro[3.4]octane-2,8-dicarboxylate (80 mg, 0.14 mmol) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl (8S)-6-benzyl-2,6-diazaspiro[3.4]octane-8-carboxylate; trifluoroacetic acid (V186717, 15.2 mg, 17.9%) and (2R,3S,4S)-4-hydroxy-2-[(4-methoxyphenyl)methyl]pyrrolidin-3-yl (8R)-6-benzyl-2,6-diazaspiro[3.4]octane-8-carboxylate; trifluoroacetic acid (V186718, 27.4 mg, 33.4%) as a white solid. MS: m/z: Calc'd for C₂₆H₃₃N₃O₄ [M+H]⁺452, found 452. Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 22% B in 10 min; Wave Length: 254 nm/220 nm nm; RT1(min): 8.5

V186717: ¹H NMR (400 MHz, DMSO-d₆) δ 7.46 (d, J=3.0 Hz, 5H), 7.24-7.14 (m, 2H), 6.90 (d, J=8.5 Hz, 2H), 5.06 (d, J=3.4 Hz, 1H), 4.35 (d, J=4.3 Hz, 1H), 4.29 (s, 2H), 4.16 (d, J=11.4 Hz, 2H), 4.10-4.06 (m, 1H), 3.95 (d, J=11.5 Hz, 2H), 3.69-3.50 (m, 4H), 3.51-3.35 (m, 2H), 3.09 (d, J=12.8 Hz, 1H), 2.97 (dd, J=14.5, 5.9 Hz, 1H), 2.82 (dd, J=14.5, 9.1 Hz, 1H).

V186718: ¹H NMR (400 MHz, DMSO-d₆) δ 7.48 (d, J=4.0, 3.3 Hz, 5H), 7.23 (dd, J=8.6, 3.0 Hz, 2H), 6.91 (d, J=8.0 Hz, 2H), 5.03 (d, J=3.4 Hz, 1H), 4.40-4.28 (m, 3H), 4.19 (dd, J=23.3, 11.5 Hz, 2H), 4.08-4.04 (m, 1H), 3.97 (dd, J=10.9, 4.0 Hz, 2H), 3.71 (d, J=2.1 Hz, 5H), 3.67-3.47 (m, 3H), 3.42 (dd, J=13.0, 4.4 Hz, 1H), 3.08 (d, J=12.8 Hz, 1H), 3.01 (dd, J=14.5, 6.0 Hz, 1H), 2.88 (dd, J=14.4, 9.0 Hz, 1H).

### Synthetic Procedure of V186760

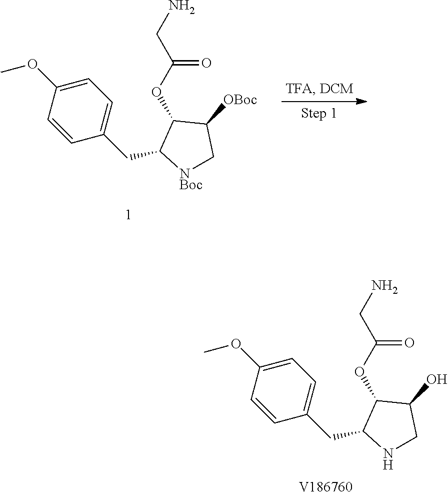

To a stirred solution of tert-butyl (2R,3S,4S)-3-[(2-aminoacetyl)oxy]-4-[(tert-butoxycarbonyl)oxy]-2-[(4-methoxyphenyl)methyl]pyrrolidine-1-carboxylate (50 mg, 0.11 mmol, 1 eq.) in DCM (5 mL) was added TFA (1 mL) at room temperature for 2 h. After completion of the reaction monitored by LCMS, the mixture was concentrated. The residue was purified Prep-HPLC to obtain (2R,3S,4S)-4-hydroxy-2-[(4-methoxyphenyl)methyl]pyrrolidin-3-yl 2-aminoacetate (22.2 mg, 75.6%) as a white solid MS: m/z: Calc'd for C₁₄H₂₀N₂O₄ [M+H]⁺281, found 281. ¹H NMR (400 MHz, DMSO-d₆) δ 7.25-7.18 (m, 2H), 6.93-6.85 (m, 2H), 4.99 (d, J=3.3 Hz, 1H), 4.25 (d, J=4.3 Hz, 1H), 4.07-3.94 (m, 3H), 3.89 (d, J=17.3 Hz, 3H), 3.51 (dd, J=12.8, 4.5 Hz, 1H), 3.09 (d, J=12.8 Hz, 1H), 2.94 (dd, J=14.1, 7.7 Hz, 2H).

Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.5

### Synthetic Procedure of V186761

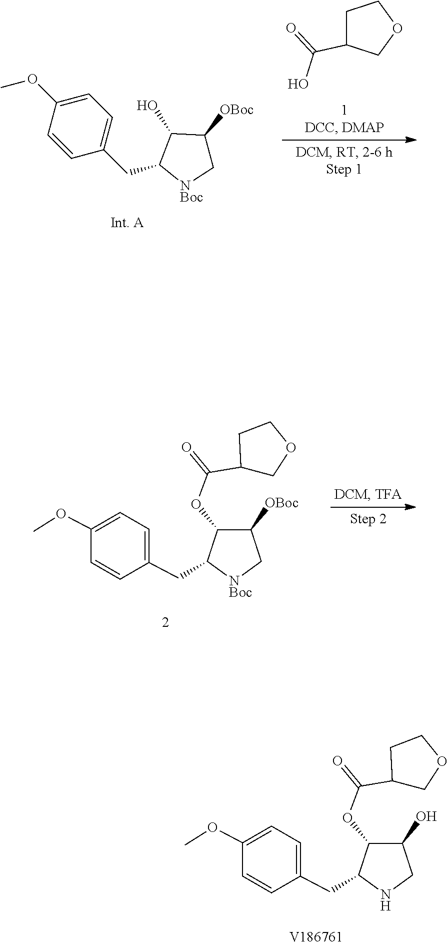

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.236 mmol, 1 eq.) and oxolane-3-carboxylic acid (54.8 mg, 0.472 mmol, 2 eq.) in DCM (5 mL) were added DCC (87.7 mg, 0.42 mmol, 1.5 eq.) and DMAP (34.6 mg, 0.28 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (10 mmol/L NH₄HCO₃ in water and acetonitrile) to afford tert-butyl (2R,3S,4S)-4-[(tert-butoxycarbonyl) oxy]-2-[(4-methoxyphenyl)methyl]-3-(oxolane-3-carbonyloxy)pyrrolidine-1-carboxylate (95 mg, 77.1%) as a white solid. MS: m/z: Calc'd for C₂₇H₃₉NO₉ [M+H]⁺522; Found 522.

Step 2: To a stirred mixture of tert-butyl (2R,3S,4S)-4-hydroxy-2-[(4-methoxyphenyl) methyl]-3-(oxolane-3-carbonyloxy)pyrrolidine-1-carboxylate (95 mg, 0.225 mmol) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl oxolane-3-carboxylate (19.3 mg, 26.59%) as an off-white solid. MS: m/z: Calc'd for C₁₇H₂₃NO₅ [M+H]⁺322; Found 322. ¹H NMR (400 MHz, DMSO-d₆) δ 9.75 (s, 1H), 9.57 (s, 1H), 7.25-7.18 (m, 2H), 6.91 (d, J=8.0 Hz, 2H), 6.08 (s, 1H), 4.91 (d, J=3.7 Hz, 1H), 4.22 (t, J=3.9 Hz, 1H), 4.04 (d, J=7.6 Hz, 1H), 3.96-3.84 (m, 2H), 3.84-3.66 (m, 5H), 3.44 (s, 2H), 3.27-3.19 (m, 1H), 3.08 (d, J=12.7 Hz, 1H), 2.95 (d, J=7.4 Hz, 2H), 2.13-2.11 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 63 mL/min mL/min; Gradient: 2% B to 27% B in 10 min; Wave Length: 254 nm/220 nm nm; RT1(min): 8.68

### Synthetic Procedure of V186763

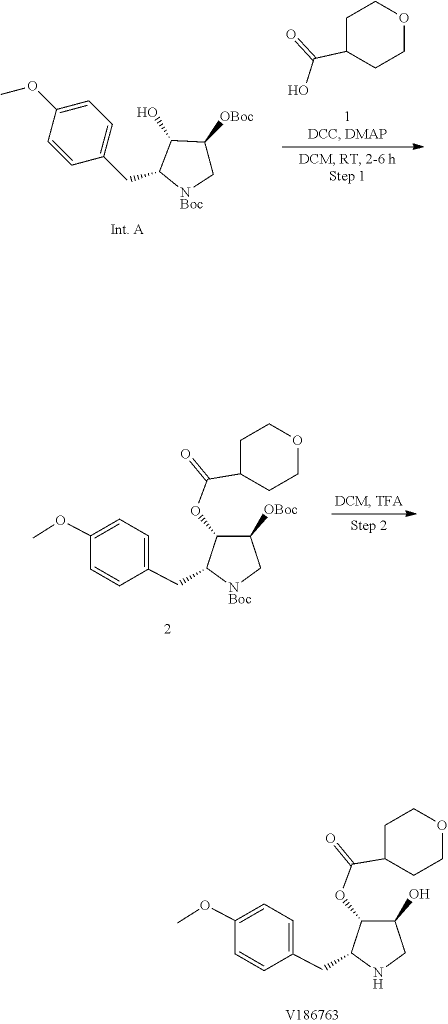

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and oxane-4-carboxylic acid (61.46 mg, 0.472 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (10 mmol/L NH₄HCO₃ in water and acetonitrile) to afford tert-butyl (2R,3S,4S)-4-[(tert-butoxycarbonyl) oxy]-2-[(4-methoxyphenyl)methyl]-3-(oxane-4-carbonyloxy)pyrrolidine-1-carboxylate (100 mg, 79.0%) as a white solid. MS: m/z: Calc'd for C₂₈H₄₁NO₉ [M+H]⁺536; Found 536.

Step 2: To a stirred mixture of tert-butyl (2R,3S,4S)-4-[(tert-butoxycarbonyl)oxy]-2-[(4-methoxyphenyl)methyl]-3-(oxane-4-carbonyloxy)pyrrolidine-1-carboxylate (90 mg, 0.168 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl oxane-4-carboxylate (21 mg, 37.19%) as a white solid. MS: m/z: Calc'd for C₁₈H₂₅NO₅ [M+H]⁺336; Found 336. ¹H NMR (400 MHz, DMSO-d₆) δ 9.48 (s, 1H), 9.15 (s, 1H), 7.21 (d, J=8.1 Hz, 2H), 6.96-6.89 (m, 2H), 6.06-6.01 (m, 1H), 4.92 (s, 1H), 4.20 (s, 1H), 4.02 (s, 1H), 3.87-3.85 (m, 2H), 3.74 (s, 3H), 3.39 (t, J=2.3 Hz, 4H), 3.09 (s, 1H), 2.91-2.85 (m, 2H), 2.80-2.72 (m, 1H), 1.86-1.85 (m, 2H), 1.73-1.66 (m, 1H), 1.65 (s, 1H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 64 mL/min mL/min; Gradient: 2% B to 29% B in 10 min; Wave Length: 254 nm/220 nm nm; RT1(min): 8.68

### Synthetic Procedure of V186772

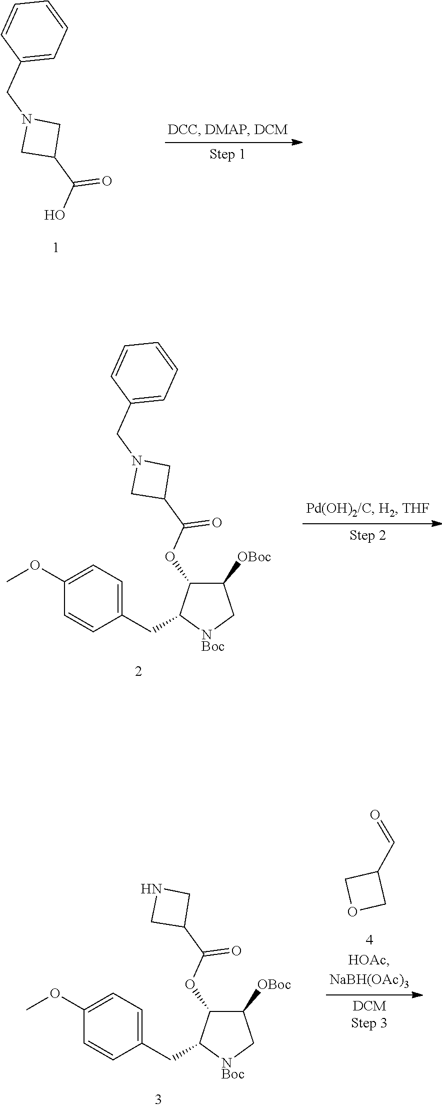

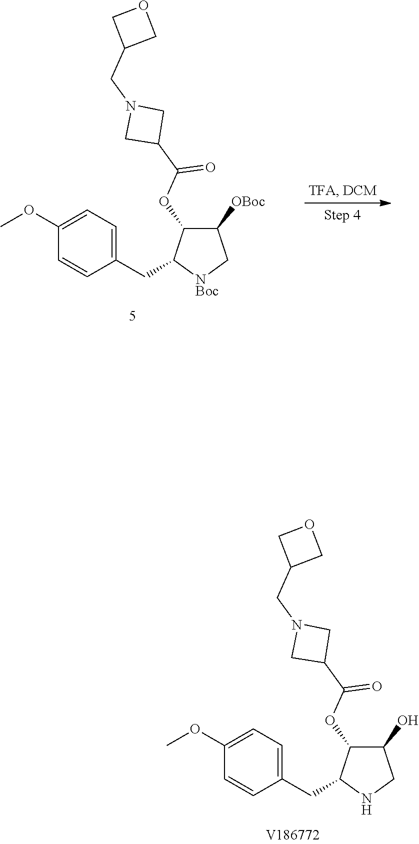

Step 1: To the solution of 1-benzylazetidine-3-carboxylic acid (406 mg, 2.13 mmol) and tert-butyl (2R,3S,4S)-4-tert-butoxycarbonyloxy-3-hydroxy-2-[(4-methoxyphenyl) methyl]pyrrolidine-1-carboxylate (600 mg, 1.42 mmol) in DCM (10 mL) were added DCC (584 mg, 2.83 mmol), DMAP (346 mg, 2.83 mmol) at 0° C., the mixture solution was stirred at room temperature overnight. LCMS showed the reaction was completed, and the reaction was concentrated, the crude was purified by silica gel column to afford (PE:EA=1:1) tert-butyl (2R,3S,4S)-3-(1-benzylazetidine-3-carbonyl)oxy-4-tert-butoxycarbonyloxy-2-[(4-methoxyphenyl)methyl]pyrrolidine-1-carboxylate (600 mg, 70.9% yield) as white solid. MS: m/z: Calc'd for C₃₃H₄₄N₂O₈ [M+H]⁺597; Found, 597.

Step 2: Under a nitrogen atmosphere, Pd(OH)₂/C (300 mg) was added to a solution of tert-butyl (2R,3S,4S)-3-(1-benzylazetidine-3-carbonyl)oxy-4-tert-butoxycarbonyloxy-2-[(4-methoxyphenyl)methyl]pyrrolidine-1-carboxylate (300 mg, 0.50 mmol) in THF (30 mL), H₂ was subsequently introduced into the reaction system, and the resulting mixture was stirred at room temperature for 2 h. Upon completion, the reaction mixture was filtered and concentrated in vacuo to afford tert-butyl (2R,3S,4S)-3-(azetidine-3-carbonyloxy)-4-tert-butoxycarbonyloxy-2-[(4-methoxyphenyl)methyl]pyrrolidine-1-carboxylate (150 mg, 59.0% yield) as white solid. MS: m/z: Calc'd for C₂₆H₃₈N₂O₈ [M+H]⁺507; Found, 507.

Step 3: To the solution of tert-butyl (2R,3S,4S)-3-(azetidine-3-carbonyloxy)-4-tert-butoxycarbonyloxy-2-[(4-methoxyphenyl)methyl]pyrrolidine-1-carboxylate (150 mg, 0.30 mmol) and oxetane-3-carbaldehyde (50 mg, 0.59 mmol) in DCM (10 mL) was added AcOH (0.05 mL, 0.59 mmol), the mixture solution was stirred at room temperature for 30 minutes and then NaBH(OAc)₃ (60 mg, 0.59 mmol) was added and stirred for another 2 h. After completion of the reaction monitored by LCMS, the mixture was concentrated. The residue was purified by TLC (PE:EA=1:1) to afford tert-butyl (2R,3S,4S)-4-tert-butoxycarbonyloxy-2-[(4-methoxyphenyl)methyl]-3-[1-(oxetan-3-ylmethyl)azetidine-3-carbonyl]oxy-pyrrolidine-1-carboxylate (60 mg, 0.10 mmol, 35.1% yield) as white solid. MS: m/z: Calc'd for C₃₀H₄₄N₂O₉ [M+H]⁺577; Found, 577.

Step 4: To a stirred solution of tert-butyl (2R,3S,4S)-4-[(tert-butoxycarbonyl)oxy]-2-[(4-methoxyphenyl)methyl]-3-[1-(oxetan-3-ylmethyl)azetidine-3-carbonyloxy]pyrrolidine-1-carboxylate (50 mg, 0.08 mmol, 1 equiv) in DCM (5 mL) was added TFA (1 mL), the mixture was stirred at room temperature for 4 h. After completion of the reaction monitored by LCMS, the mixture was concentrated. The residue was purified prep-HPLC to afford (2R,3S,4S)-4-hydroxy-2-[(4-methoxyphenyl)methyl]pyrrolidin-3-yl 1-(oxetan-3-ylmethyl)azetidine-3-carboxylate (11.4 mg, 33.2%) as off white solid. MS: m/z: Calc'd for C₂₀H₂₈N₂O₅ [M+H]⁺377; Found, 377. ¹H NMR (400 MHz, DMSO-d₆) δ 7.22 (d, J=8.2 Hz, 2H), 6.91 (d, J=8.2 Hz, 2H), 5.01-4.94 (m, 1H), 4.67-4.60 (m, 2H), 4.39-4.23 (m, 7H), 4.08-3.99 (m, 1H), 3.85-3.72 (m, 4H), 3.59-3.52 (m, 3H), 3.26-3.15 (m, 1H), 3.12-3.05 (m, 1H), 3.00-2.93 (m, 1H), 2.91-2.83 (m, 1H). Prep-HPLC-conditions: Column: XBridge Prep Shield RP C18 Column, 19*250 mm, 5 μm; Mobile Phase A: Water (0.1% TFA), Mobile Phase B: ACN; Flow rate: 25 mL/min mL/min; Gradient: 0% B to 13% B in 15 min; Wave Length: 254 nm/220 nm nm; RT1(min): 13.45

### Synthetic Procedure of V186774

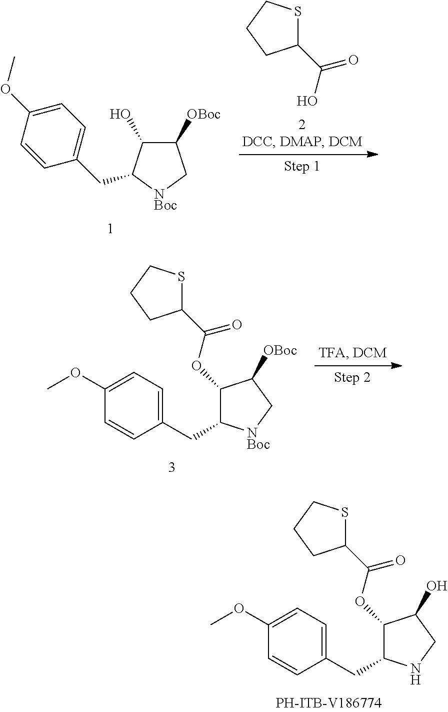

Step 1: To a stirred solution of tetrahydrothiophene-2-carboxylic acid (62.4 mg, 0.47 mmol, 2 eq.) and tert-butyl (2R,3S,4S)-4-[(tert-butoxycarbonyl)oxy]-3-hydroxy-2-[(4-methoxyphenyl) methyl]pyrrolidine-1-carboxylate (100 mg, 0.24 mmol, 1 eq.) in DCM (10 mL) were added DCC (73.1 mg, 0.35 mmol, 1.5 eq.) and DMAP (28.9 mg, 0.24 mmol, 1 eq.) at 0° C. The resulting mixture was stirred at r.t. for 12 h. After completion of reaction monitored by LCMS. The reaction mixture was quenched with water and extracted with EA. The extracts were washed with brine, dried over anhydrous sodium sulfate, filtered and concentrated. The residue was purified by Prep-HPLC to obtain tert-butyl (2R,3S,4S)-4-[(tert-butoxycarbonyl)oxy]-2-[(4-methoxyphenyl)methyl]-3-(thiolane-2-carbonyloxy)pyrrolidine-1-carboxylate (80 mg, 0.15 mmol, 57.3%) as a white solid. MS: m/z: Calc'd for C₂₇H₃₉NO₈S [M+H−56−56]⁺426, found 426.

Step 2: To a stirred solution of tert-butyl (2R,3S,4S)-4-[(tert-butoxycarbonyl)oxy]-2-[(4-methoxyphenyl)methyl]-3-(thiolane-2-carbonyloxy)pyrrolidine-1-carboxylate (80 mg, 0.15 mmol, 1 eq) in DCM (2 mL) was added TFA (1 mL) dropwise at 0° C. The resulting mixture was stirred at r.t. for 3 h. After completion of reaction monitored by LCMS. The resulting mixture was concentrated under vacuum. The crude product (75 mg) was purified by Prep-HPLC to afford (2R,3S,4S)-4-hydroxy-2-[(4-methoxyphenyl)methyl]pyrrolidin-3-yl thiolane-2-carboxylate (22.3 mg, 44.4%) as a light yellow semi-solid. MS: m/z: Calc'd for C₁₇H₂₃NO₄S [M+H]⁺338, found 338. ¹H NMR (400 MHz, Methanol-d₄) δ 7.32-7.20 (m, 2H), 6.97-6.90 (m, 2H), 5.11-5.04-5.01 (m, 1H), 4.38-4.29 (m, 1H), 4.25-4.05 (m, 2H), 3.79 (s, 3H), 3.64-3.54 (m, 1H), 3.27-3.19 (m, 1H), 3.20-2.87 (m, 4H), 2.45-2.32 (m, 1H), 2.29-2.11 (m, 2H), 2.10-1.97 (m, 1H). Column: XselectCSH Prep OBD C18 Column, 30*150 mm, 5 μm; Mobile Phase A: Water(0.05% TFA)Mobile Phase B: ACN; Flow rate: 60 mL/min mL/min; Gradient: 8% B to 38% B in 10 min; Wave Length: 254 nm/220 nm nm; RT1(min): 8.68

### Synthetic Procedure of V186775

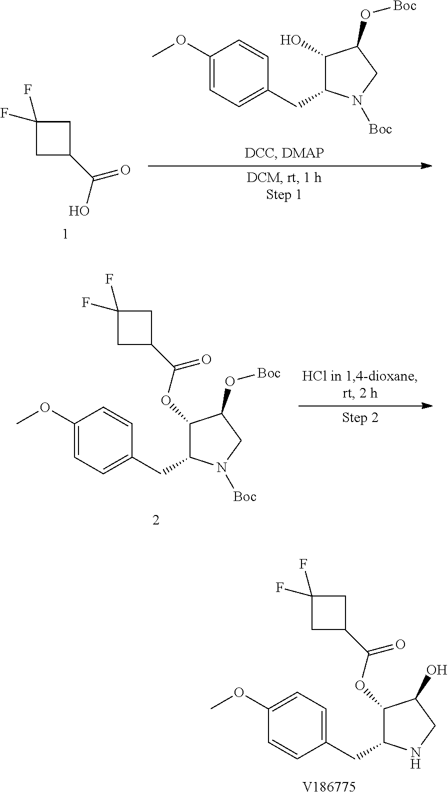 [SMILES: O=C(O)C1CC(F)(F)C1 ; COc1ccc(C[C@@H]2[C@H](O)[C@@H](OC(=O)OC(C)(C)C)CN2C(=O)OC(C)(C)C)cc1 ; COc1ccc(C[C@@H]2[C@H](OC(=O)C3CC(F)(F)C3)[C@@H](OC(=O)OC(C)(C)C)CN2C(=O)OC(C)(C)C)cc1 ; COc1ccc(C[C@H]2NC[C@H](O)[C@H]2OC(=O)C2CC(F)(F)C2)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (120.0 mg, 0.28 mmol, 1.0 eq.) and 3,3-difluorocyclobutane-1-carboxylic acid (50.0 mg, 0.37 mmol, 1.3 eq.) in DCM (5.0 mL) were added DCC (58.5 mg, 0.28 mmol, 1.0 eq.) and DMAP (34.6 mg, 0.28 mmol, 1.0 eq.) at room temperature. The resulting mixture was stirred for 1 h. LCMS showed completion of starting material. The resulting mixture was concentrated under reduced pressure. The residue was purified by reversed-phase flash chromatography with the following conditions: column, C18 silica gel; mobile phase, MeCN in Water (0.1% TFA), 10% to 80% gradient in 20 min; detector, UV 254 nm, to get tert-butyl (2R,3S,4S)-4-[(tert-butoxycarbonyl)oxy]-3-(3,3-difluorocyclobutanecarbonyloxy)-2-[(4-methoxyphenyl)methyl]pyrrolidine-1-carboxylate (150.0 mg, 97.8%) as a white solid. MS: m/z: Calc'd for C₂₇H₃₇F₂NO₈, [M+H−56−56]⁺430; Found 430.

Step 2: To a solution of tert-butyl (2R,3S,4S)-4-[(tert-butoxycarbonyl)oxy]-3-(3,3-difluorocyclobutanecarbonyloxy)-2-[(4-methoxyphenyl)methyl]pyrrolidine-1-carboxylate (140.0 mg, 0.26 mmol, 1.0 eq.) in 1,4-dioxane (5.0 mL) was added HCl-dioxane (1M, 5 mL) at room temperature and the resulting mixture was stirred for 2 h. LCMS showed completion of starting material. The resulting mixture was concentrated under reduced pressure. The crude product (150.0 mg) was purified by Prep-HPLC with the following conditions (Column: Xselect CSH Prep C18 Column, 30*150 mm, 5 μm; Mobile Phase A: Water (0.05% TFA), Mobile Phase B: ACN; Flow rate: 65 mL/min; Gradient: 6% B to 36% B in 10 min; Wave Length: 254 nm/220 nm nm; RT1(min): 8.68) to afford (2R,3S,4S)-4-hydroxy-2-[(4-methoxyphenyl)methyl]pyrrolidin-3-yl 3,3-difluorocyclobutane-1-carboxylate; trifluoroacetic acid (68.1 mg, 77.2%) as a white solid. MS: m/z: Calc'd for C₁₇H₂₁F₂NO₄ [M+H]⁺342, Found 342. ¹H NMR (400 MHz, Methanol-d₄) δ 7.26-7.19 (m, 2H), 6.97-6.90 (m, 2H), 5.13 (d, J=3.4 Hz, 1H), 4.38 (d, J=4.4 Hz, 1H), 4.21-4.19 (m, 1H), 3.80 (s, 3H), 3.59 (dd, J=12.7, 4.5 Hz, 1H), 3.21 (dd, J=11.9, 8.7 Hz, 2H), 3.09 (dd, J=14.3, 6.8 Hz, 1H), 3.03-2.83 (m, 5H). Prep-HPLC conditions: Column: Xselect CSH Prep C18 Column, 30*150 mm, 5 μm; Mobile Phase A: Water (0.05% TFA), Mobile Phase B: ACN; Flow rate: 65 mL/min; Gradient: 6% B to 36% B in 10 min; Wave Length: 254 nm/220 nm nm; RT1(min): 8.68.

### Synthetic Procedure of V186781

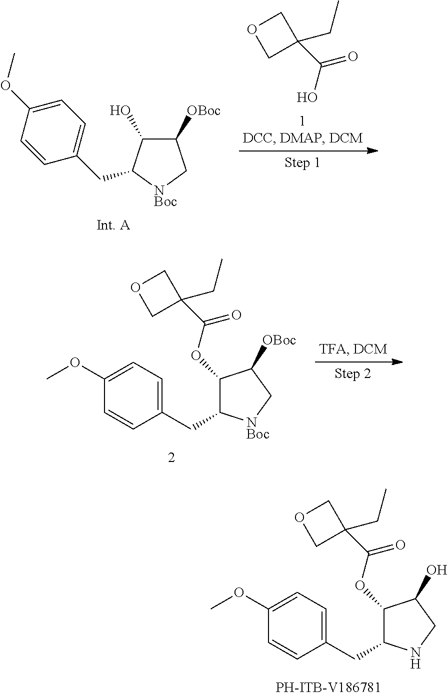

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-ethyloxetane-3-carboxylic acid (61.4 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-(3-ethyloxetane-3-carbonyloxy)-2-[(4-methoxyphenyl)methyl]pyrrolidine-1-carboxylate (65 mg, 51.39%) as a white solid. MS: m/z: Calc'd for C₂₈H₄₁NO₉ [M+H]⁺536; Found 536.

Step 2: To a stirred mixture of tert-butyl (2R,3S,4S)-4-[(tert-butoxycarbonyl)oxy]-3-(3-ethyloxetane-3-carbonyloxy)-2-[(4-methoxyphenyl)methyl]pyrrolidine-1-carboxylate (80 mg, 0.149 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-ethyloxetane-3-carboxylate (24.4 mg, 48.5%) as a white solid. ¹H NMR (400 MHz, DMSO-d₆) δ 7.11 (d, J=8.2 Hz, 2H), 6.84-6.81 (m, 2H), 5.19 (s, 1H), 4.80-4.71 (m, 3H), 4.41 (t, J=6.4 Hz, 2H), 3.97-3.91 (m, 1H), 3.72 (s, 3H), 3.38-3.36 (m, 1H), 3.17-3.15 (m, 1H), 2.62 (d, J=7.2 Hz, 2H), 2.59-2.51 (m, 1H), 2.01-1.98 (m, 2H), 0.84 (t, J=7.4 Hz, 3H). Prep-HPLC-conditions: Column: XSelect CSH Prep C18 OBD Column, 5 μm, 30*150 mm; Mobile Phase A:Water (0.05% NH₄HCO₃), Mobile Phase B:ACN; Flow rate:60 mL/min; Gradient:12 B to 42 B in 22 min; 254/220 nm; RT1:6.1; RT2:; Injection Volumn: ml; Number Of Runs:;

### Synthetic Procedure of V186782

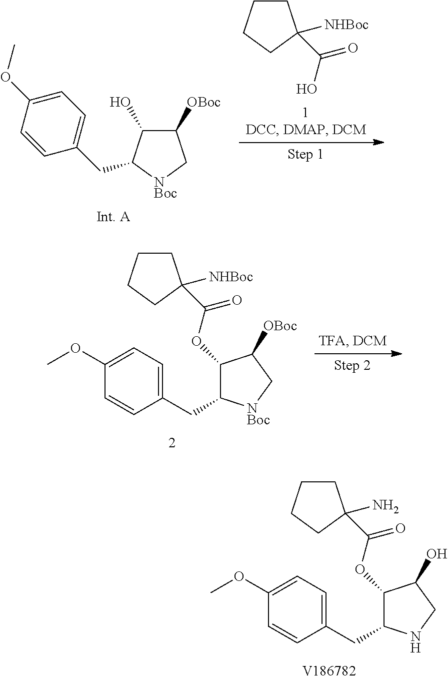

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1 eq.) and 1-[(tert-butoxycarbonyl)amino]cyclopentane-1-carboxylic acid (64.9 mg, 0.28 mmol, 1.5 eq.) in DCM (5 mL) were added DCC (77.9 mg, 0.37 mmol, 1.5 eq.) and DMAP (46.1 mg, 0.37 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-3-{1-[(tert-butoxycarbonyl)amino]cyclopentanecarbonyloxy}-4-[(tert-butoxycarbonyl)oxy]-2-[(4-methoxyphenyl)methyl]pyrrolidine-1-carboxylate (120 mg, 100.08%) as off white solid. MS: m/z: Calc'd for C₃₃H₅₀N₂O₁₀ [M+Na]⁺657; Found 657.

Step 2: To a stirred mixture of tert-butyl (2R,3S,4S)-3-{1-[(tert-butoxycarbonyl) amino]cyclopentanecarbonyloxy}-4-[(tert-butoxycarbonyl)oxy]-2-[(4-methoxyphenyl) methyl]pyrrolidine-1-carboxylate (100 mg, 0.16 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 1-aminocyclopentane-1-carboxylate; trifluoroacetic acid salt (22.7 mg, 32.0%) as a brown solid. MS: m/z: Calc'd for C₁₈H₂₆N₂O₄ [M+H]⁺335; Found 335. ¹H NMR (400 MHz, DMSO-d₆) (9.67 (s, 2H), 8.67 (s, 3H), 7.23 (dd, J=8.8, 2.8 Hz, 2H), 6.97-6.89 (m, 2H), 6.28-5.98 (m, 1H), 5.10-5.02 (m, 1H), 4.26-4.09 (m, 2H), 3.75 (d, J=1.9 Hz, 3H), 3.68-3.48 (m, 2H), 3.11-3.09 (m, 1H), 2.99 (s, 2H), 2.27-2.12 (m, 2H), 1.95-1.85 (m, 6H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm nm; RT1(min): 8.68

### Synthetic Procedure of V186784

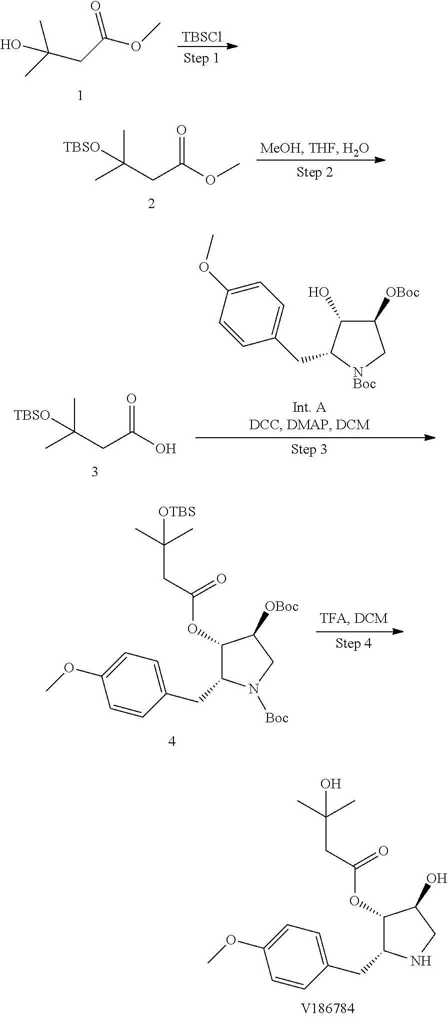

Step 1: To a stirred solution of methyl 3-hydroxy-3-methylbutanoate (500 mg, 3.78 mmol, 1 eq.) in DCM (30 mL) were added tert-butyldimethylsilyl trifluoromethanesulfonate (2500.1 mg, 9.45 mmol, 2.5 eq.), pyridine (897.78 mg, 11.349 mmol, 3.0 eq.) at 0° C., the resulting solution was stirred at room temperature for overnight. Upon completion, the reaction mixture was concentrated in vacuo and purified by silica gel column chromatography (PE:EA=20:1 as eluent) to afford methyl 3-[(tert-butyldimethylsilyl)oxy]-3-methylbutanoate (700 mg, 75.0%) as colorless oil. The product was confirmed by ¹H NMR.

Step 2: To a stirred solution of methyl 3-[(tert-butyldimethylsilyl)oxy]-3-methylbutanoate (300 mg, 1.21 mmol, 1 eq.) in THF (10 mL), MeOH (4 mL), H₂O (4 mL) was added LiOH·H₂O (153.25 mg, 3.651 mmol, 3 eq.),. The resulting mixture was stirred at room temperature for 4 h. Upon completion, the reaction mixture was concentrated in vacuo. The resulting residue was dissolved with DMSO and purified by a reversed-phase column chromatography (0.05% TFA in water and acetonitrile) to afford (3,6-dioxopiperazin-2-yl) acetic acid (150 mg, 53.2%) as a white solid. MS: m/z: Calc'd for C₁₁H₂₄O₃Si[M−H]⁻231; Found 231.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.189 mmol, 1 eq.) and 3-[(tert-butyldimethylsilyl)oxy]-3-methylbutanoic acid (87.8 mg, 0.37 mmol, 2 eq.) in DCM (5 mL) were added DCC (58.4 mg, 0.28 mmol, 1.5 eq.) and DMAP (23.0 mg, 0.18 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-({3-[(tert-butyldimethylsilyl)oxy]-3-methylbutanoyl}oxy)-2-[(4-methoxyphenyl)methyl]pyrrolidine-1-carboxylate (100 mg, 82.9%) as a white solid. MS: m/z: Calc'd for C₃₃H₅₅NO₉Si[M+H]⁺638; Found 638.

Step 4: To a stirred mixture of tert-butyl (2R,3S,4S)-4-[(tert-butoxycarbonyl)oxy]-3-({3-[(tert-butyldimethylsilyl)oxy]-3-methylbutanoyl}oxy)-2-[(4-methoxyphenyl)methyl]pyrrolidine-1-carboxylate (70 mg, 0.11 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-hydroxy-3-methylbutanoate (13.7 mg, 38.14%) as a white solid. MS: m/z: Calc'd for C₁₇H₂₅NO₅ [M+H]⁺324; Found, 324. ¹H NMR (400 MHz, Methanol-d₄) δ 7.21-7.11 (m, 2H), 6.89-6.78 (m, 2H), 4.83 (m, 1H), 4.16-4.14 (m, 1H), 3.76 (s, 3H), 3.61-3.52 (m, 1H), 3.42-3.34 (m, 1H), 2.90-2.71 (m, 3H), 1.35 (s, 2H), 1.34 (s, 6H). Prep-HPLC-conditions: Column: XSelect CSH Prep C18 OBD Column, 5 μm, 30*150 mm; Mobile Phase A:Water (NH₄HCO₃), Mobile Phase B:ACN; Flow rate:25 mL/min; Gradient:8 B to 29 B in 22 min; 254/220 n; RT1:7.65; RT2:; Injection Volumn: ml; Number Of Runs:;

### Synthetic Procedure of V186785

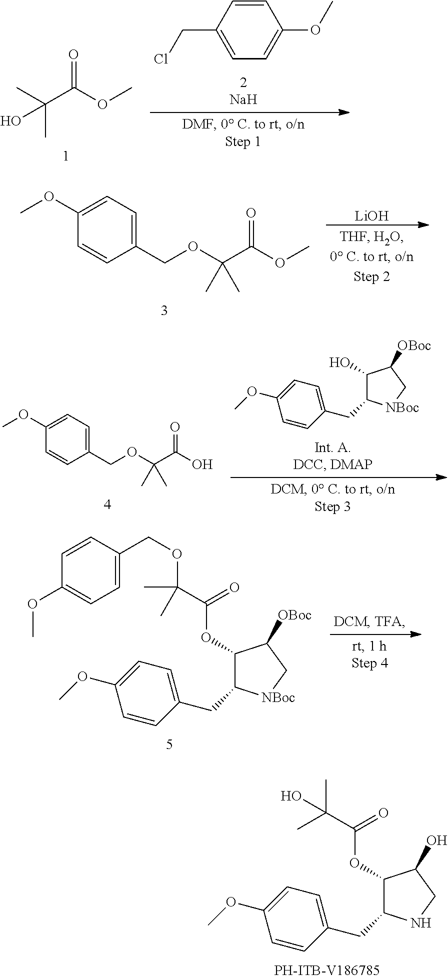

Step 1: To a stirred solution of methyl 2-hydroxy-2-methylpropanoate (150 mg, 1.27 mmol, 1 eq.) in DMF (3 mL) were added NaH (60.9 mg, 2.54 mmol, 2 eq.) and 4-methoxybenzyl chloride (397.7 mg, 2.54 mmol, 2 eq.) at 0° C. The reaction was stirred at r.t. for overnight under nitrogen atmosphere. After completion of the reaction monitored by LCMS, the reaction was quenched with water at 0° C. The resulting mixture was extracted with EtOAc (2×100 mL). The combined organic layers were washed with water (2×100 mL), dried over anhydrous Na₂SO₄. After filtration, the filtrate was concentrated under reduced pressure. The crude product was purified by reverse phase flash with the following conditions (0.5% NH₄HCO₃ in H₂O/ACN) to afford methyl 2-[(4-methoxyphenyl)methoxy]-2-methylpropanoate (200 mg, 66.1%) as a yellow solid. MS: m/z: Calc'd for C₈H₁₂N₂O₄[M−H]⁻199; Found, 199.

Step 2: To a stirred solution of methyl 2-[(4-methoxyphenyl)methoxy]-2-methylpropanoate (200 mg, 0.83 mmol, 1 eq.) in THF (1.5 mL) and H₂O (1.5 mL) was added LiOH (40.2 mg, 1.67 mmol, 2 eq.) at 0° C. The reaction was stirred at room temperature for 2 h. After completion of the reaction monitored by LCMS, the resulting mixture was acidified and extracted with CH₂Cl₂ (2×100 mL). The combined organic layers were washed with water (2×100 mL), dried over anhydrous Na₂SO₄. After filtration, the filtrate was concentrated under reduced pressure. The resulting mixture was concentrated under reduced pressure to afford 2-[(4-methoxyphenyl)methoxy]-2-methylpropanoic acid (157 mg, 83.4%) as a brown solid. MS: m/z: Calc'd for C₁₂H₁₆O₄[M−H]⁺223; Found, 223.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 70 mg, 0.16 mmol, 1 eq.) and 2-[(4-methoxyphenyl)methoxy]-2-methylpropanoic acid (44.4 mg, 0.19 mmol, 1.2 eq.) in DCM (5 mL, 219.84 mmol) were added DCC (51.1 mg, 0.24 mmol, 1.5 eq.) and DMAP (20.1 mg, 0.16 mmol, 1 eq.) at 0° C. The reaction was stirred at room temperature for overnight under nitrogen atmosphere. After completion of the reaction monitored by LCMS, the resulting mixture was concentrated under reduced pressure. The residue was purified by Prep-TLC (PE/EA 5:1) to afford tert-butyl (2R,3S,4S)-4-[(tert-butoxycarbonyl)oxy]-3-({2-[(4-methoxyphenyl)methoxy]-2-methylpropanoyl}oxy)-2-[(4-methoxyphenyl)methyl]pyrrolidine-1-carboxylate (53.2 mg, 51.1%) as an off-white oil. MS: m/z: Calc'd for C₃₄H₄₇NO₁₀ [M+H⁺22]⁺652; Found, 652.

Step 4: To a stirred solution of tert-butyl (2R,3S,4S)-4-[(tert-butoxycarbonyl)oxy]-3-({2-[(4-methoxyphenyl)methoxy]-2-methylpropanoyl}oxy)-2-[(4-methoxyphenyl)methyl]pyrrolidine-1-carboxylate (50 mg, 0.07 mmol, 1 eq.) in DCM (3 mL) was added TFA (1 mL, 13.46 mmol, 169.57 eq.) at 0° C. The reaction was stirred at room temperature for 2 h. After completion of the reaction monitored by LCMS, the resulting mixture was concentrated under reduced pressure. The crude product (75 mg) was purified by Prep-HPLC to obtained (2R,3S,4S)-4-hydroxy-2-[(4-methoxyphenyl)methyl]pyrrolidin-3-yl 2-hydroxy-2-methylpropanoate; trifluoroacetic acid (13.2 mg, 38.9%) as yellow semi-solid. MS: m/z: Calc'd for C₁₆H₂₃NO₅ [M+H]⁺310; Found, 310. ¹H NMR (400 MHz, DMSO-d₆) δ 9.49 (s, 1H), 9.12 (s, 1H), 7.23-7.16 (m, 2H), 6.99-6.87 (m, 2H), 6.06 (d, J=3.4 Hz, 1H), 5.42 (s, 1H), 4.92-4.86 (m, 1H), 4.15 (t, J=3.9 Hz, 1H), 4.02 (s, 1H), 3.74 (s, 3H), 3.44 (s, 1H), 3.09 (d, J=12.7 Hz, 1H), 2.94-2.90 (m, 2H), 1.39 (d, J=10.2 Hz, 6H). Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 10% B to 25% B in 10 min; Wave Length: 254 nm/220 nm nm; RT1(min): 8.68.

### Synthetic Procedure of V186809

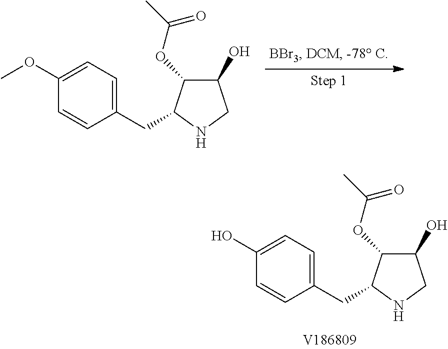 [SMILES: COc1ccc(C[C@H]2NC[C@H](O)[C@H]2OC(C)=O)cc1 ; CC(=O)O[C@@H]1[C@@H](O)CN[C@@H]1Cc1ccc(O)cc1]

To a stirred solution of anisomycin (500 mg, 1.88 mmol) in Dichloromethane (8 mL) was added Boron tribromide (5.6 mL, 3 eq) at −78° C. The reaction mixture was stirred at −78° C. for 2 h and warmed to room temperature. The mixture was stirred for 1 h at room temperature and quenched by MeOH (5 mL). The solution was concentrated, and the resulting residue was purified by a reversed-phase column to obtained 500 mg of crude. 100 mg of crude was further purified by Prep-HPLC to obtain (2R,3S,4S)-4-hydroxy-2-[(4-hydroxyphenyl)methyl]pyrrolidin-3-yl acetate (11.2 mg, 2.32%) as a white solid. MS: m/z: Calc'd for C₁₃H₁₇NO₄ [M+H]⁺252; found 252. ¹H NMR (400 MHz, Methanol-d4) δ 7.19-7.09 (m, 2H), 6.83-6.75 (m, 2H), 5.08 (d, J=3.4 Hz, 1H), 4.39-4.33 (m, 1H), 4.21-4.11 (m, 1H), 3.61 (dd, J=12.7, 4.5 Hz, 1H), 3.21 (d, J=12.7 Hz, 1H), 3.05 (dd, J=14.2, 6.8 Hz, 1H), 2.94 (dd, J=14.2, 8.8 Hz, 1H), 2.19 (d, J=0.9 Hz, 3H). Prep-HPLC purification condition: Column: XBridge Prep OBD C18 Column, 19*250 mm, 5 μm; Mobile Phase A: Water (0.05% TFA), Mobile Phase B: ACN; Flow rate: 25 mL/min mL/min; Gradient: 2% B to 22% B in 8 min; Wave Length: 254 nm/220 nm nm; RT1(min): 7.42

### Synthetic Procedure of V186810

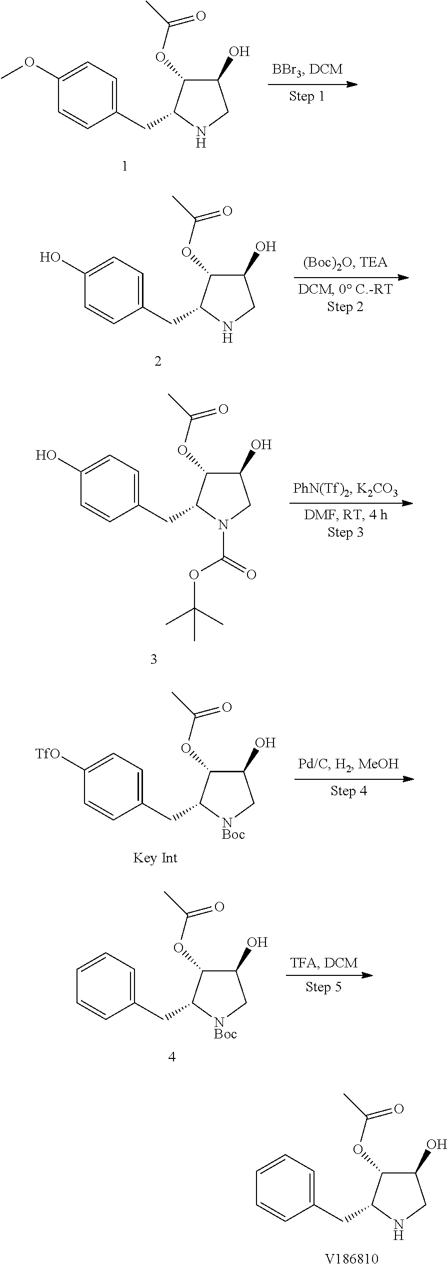 [SMILES: COc1ccc(C[C@H]2NC[C@H](O)[C@H]2OC(C)=O)cc1 ; CC(=O)O[C@@H]1[C@@H](O)CN[C@@H]1Cc1ccc(O)cc1 ; CC(=O)O[C@H]1[C@@H](Cc2ccc(O)cc2)N(C(=O)OC(C)(C)C)C[C@@H]1O ; CC(=O)O[C@H]1[C@@H](Cc2ccc(OS(=O)(=O)C(F)(F)F)cc2)N(C(=O)OC(C)(C)C)C[C@@H]1O ; CC(=O)O[C@H]1[C@@H](Cc2ccccc2)N(C(=O)OC(C)(C)C)C[C@@H]1O ; CC(=O)O[C@@H]1[C@@H](O)CN[C@@H]1Cc1ccccc1]

Step 1: To a stirred solution of anisomycin (1.5 g, 5.654 mmol, 1 equiv) in Dichloromethane (8 mL) was added Boron tribromide (16.8 mL, 3 equiv) at −78° C. The reaction mixture was stirred at −78° C. for 2 h and warmed to room temperature. The mixture was stirred for 1 h at room temperature and quenched by saturated NaHCO₃ solution. The DCM was removed, and the solution was lyophilized to obtain (2R,3S,4S)-4-hydroxy-2-[(4-hydroxyphenyl)methyl]pyrrolidin-3-yl acetate (1.8 g, 85.41%) as a crude. MS: m/z: Calc'd for C₁₃H₁₇NO₄ [M+H]⁺252; found 252.

Step 2: To a stirred solution of (2R,3S,4S)-4-hydroxy-2-[(4-hydroxyphenyl)methyl]pyrrolidin-3-yl acetate (1.8 g, 7.16 mmol, 1 equiv) and triethylamine (2.54 g, 25.07 mmol, 3.5 equiv) in DCM (30 mL) was added di-tert-butyl dicarbonate (1.88 g, 8.59 mmol, 1.2 equiv) at 0° C. The mixture was stirred at room temperature for 3 h. After completion of the reaction monitored by LCMS, the reaction mixture was filtrated. The filtrate was concentrated. The residue was purified by a reversed-phase column to obtain tert-butyl (2R,3S,4S)-3-(acetyloxy)-4-hydroxy-2-[(4-hydroxyphenyl)methyl]pyrrolidine-1-carboxylate (1.7 g, 67.54%) as a white solid. MS: m/z: Calc'd for C₁₈H₂₅NO₆ [M−H]⁻350; found 350.

Step 3: To a stirred solution of tert-butyl (2R,3S,4S)-3-(acetyloxy)-4-hydroxy-2-[(4-hydroxyphenyl) methyl]pyrrolidine-1-carboxylate (1.7 g, 4.83 mmol,) and Potassium carbonate (2.01 g, 14.51 mmol, 3 equiv) in DMF (16 mL) was added 1,1,1-trifluoro-N-phenyl-N-trifluoromethanesulfonyl methanesulfonamide (2.25 g, 6.28 mmol, 1.3 equiv) at 0° C. The reaction mixture was stirred at room temperature for 2 h. After completion of the reaction monitored by LCMS, the reaction mixture was filtrated. The filtrate was injected into a reversed-phase column and purified to obtain tert-butyl (2R,3S,4S)-3-(acetyloxy)-4-hydroxy-2-{[4-(trifluoromethanesulfonyloxy)phenyl]methyl}pyrrolidine-1-carboxylate (1.5 g, 64.13%) as a white solid. MS: m/z: Calc'd for C₁₉H₂₄F₃NO₈S [M+NH₄]⁺501; found 501.

Step 4: To a stirred solution of tert-butyl (2R,3S,4S)-3-(acetyloxy)-4-hydroxy-2-{[4-(trifluoro methanesulfonyloxy)phenyl]methyl}pyrrolidine-1-carboxylate (100 mg, 0.20 mmol, 1 equiv) in methanol (6 mL) was added Palladium 10% on Carbon (100 mg) under N₂. Then the hydrogen was subsequently injected into the mixture. The at room temperature for 2 h. After completion of the reaction monitored by LCMS, the mixture was filtrated. The filtrate was concentrated to obtain tert-butyl (2S,3R,4R)-3-(acetyloxy)-2-benzyl-4-hydroxypyrrolidine-1-carboxylate (47 mg, 67.75%) as a white solid. MS: m/z: Calc'd for C₁₈H₂₅NO₅[M−56+H]⁺280; found 280.

Step 5: To a stirred solution of tert-butyl (2R,3S,4S)-3-(acetyloxy)-2-benzyl-4-hydroxypyrrolidine-1-carboxylate (42 mg, 0.125 mmol, 1 equiv) in DCM (5 mL) was added trifluoroacetic acid (1 mL). The reaction mixture was stirred at room temperature for 2 h. LCMS showed the starting material was consumed completely. The mixture was concentrated. The resulting residue was purified by Prep-HPLC to obtain (2R,3S,4S)-2-benzyl-4-hydroxy pyrrolidin-3-yl acetate (10.6 mg) as an off-white solid. MS: m/z: Calc'd for C₁₃H₁₇NO₃ [M+H]⁺236; found 236. ¹H NMR (400 MHz, Methanol-d₄) δ 7.39 (t, J=7.5 Hz, 2H), 7.33 (d, J=7.3 Hz, 3H), 5.09 (d, J=3.4 Hz, 1H), 4.38 (d, J=4.4 Hz, 1H), 4.28-4.19 (m, 1H), 3.63 (dd, J=12.7, 4.5 Hz, 1H), 3.26-3.12 (m, 2H), 3.06 (dd, J=14.2, 8.6 Hz, 1H), 2.20 (s, 3H).

Prep-HPLC purification condition: Column: Xselect CSH Prep C18 Column, 30*150 mm, 5 μm; Mobile Phase A: Water (0.1% TFA), Mobile Phase B: ACN; Flow rate: 60 mL/min mL/min; Gradient: 2% B to 19% B in 10 min; Wave Length: 254 nm/220 nm nm; RT1(min): 8.68.

### Synthetic Procedure of V186812

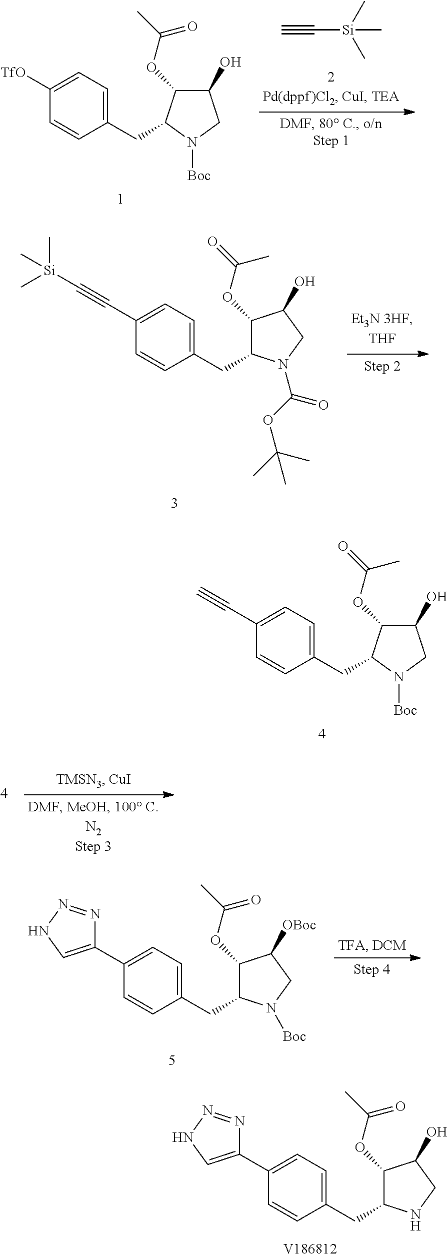

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (200 mg, 0.41 mmol, 1 eq.) and trimethylsilylacetylene (121.9 mg, 1.24 mmol, 3 eq.) in DMF (10 mL) were added TEA (167.5 mg, 1.66 mmol, 4 eq.), CuI (7.9 mg, 0.04 mmol, 0.1 eq.) and Pd(dppf)Cl₂·CH₂Cl₂ (50.6 mg, 0.06 mmol, 0.15 eq.) at room temperature. The reaction mixture purged with N₂ and heated at 80° C. for 12 h. After completion of reaction monitored by LCMS. The resulting mixture was filtered, the filter cake was washed with EA (3×20 mL). The filtrate was concentrated under reduced pressure. The residue was purified by Prep-TLC (PE/EA 2:1) to afford tert-butyl (2R,3S,4S)-3-(acetyloxy)-4-hydroxy-2-({4-[2-(trimethylsilyl)ethynyl]phenyl}methyl) pyrrolidine-1-carboxylate (160 mg, 89.6%) as a yellow oil. MS: m/z: Calc'd for C₂₃H₃₃NO₅Si[M+H−56]⁺376, found [M+H−56]⁺376.

Step 2: To a stirred solution of tert-butyl (2R,3S,4S)-3-(acetyloxy)-4-hydroxy-2-({4-[2-(trimethylsilyl)ethynyl]phenyl}methyl)pyrrolidine-1-carboxylate (170 mg, 0.39 mmol, 1 eq.) in THF (5 mL) was added Et₃N·3HF(190.5 mg, 1.18 mmol, 3.0 eq.) at r.t. The resulting mixture was stirred at 80° C. for 3 h. After completion of reaction monitored by LCMS. The reaction mixture was concentrated. The residue was purified by Prep-TLC (PE/EA 3:1) to afford tert-butyl (2R,3S,4S)-3-(acetyloxy)-2-[(4-ethynylphenyl)methyl]-4-hydroxypyrrolidine-1-carboxylate (135 mg, 95.4%) as a light yellow solid. MS: m/z: Calc'd for C₂₀H₂₅NO₅ [M+H−56]⁺304, found [M+H−56]⁺304.

Step 3: To a stirred solution of tert-butyl (2R,3S,4S)-3-(acetyloxy)-2-[(4-ethynylphenyl) methyl]-4-hydroxypyrrolidine-1-carboxylate (120 mg, 0.33 mmol, 1 eq.) and trimethylsilyl azide (76.9 mg, 0.67 mmol, 2 eq.) in DMF (5 mL) and MeOH (5 mL) was added CuI (63.6 mg, 0.33 mmol, 1 eq.) at room temperature. The solution was stirred at 100° C. for 12 h under nitrogen atmosphere. After completion of reaction monitored by LCMS. The resulting mixture was filtered, the filter cake was washed with EA (3×20 mL). The filtrate was concentrated under reduced pressure. The residue was purified by reversed-phase flash to obtain tert-butyl (2R,3S,4S)-3-(acetyloxy)-4-hydroxy-2-{[4-(1H−1,2,3-triazol-4-yl)phenyl]methyl}pyrrolidine-1-carboxylate (80 mg, 59.5%) as a yellow oil. MS: m/z: Calc'd for C₂₅H₃₄N₄O₇ [M+H−56]⁺347, found [M+H−56]⁺347.

Step 4: To a stirred solution of tert-butyl (2R,3S,4S)-3-(acetyloxy)-4-hydroxy-2-{[4-(1H-1,2,3-triazol-4-yl)phenyl]methyl}pyrrolidine-1-carboxylate (75 mg, 0.19 mmol, 1 eq.) in 1,4-dioxane (4 mL) was added HCl-1,4-dioxane (4M, 4 mL) dropwise at 0° C. The solution was stirred at room temperature for 12 h. After completion of reaction monitored by LCMS. The resulting mixture was concentrated under reduced pressure. The crude product (50 mg) was purified by Prep-HPLC to afford (2R,3S,4S)-4-hydroxy-2-{[4-(1H−1,2,3-triazol-4-yl)phenyl]methyl}pyrrolidin-3-yl acetate; trifluoroacetic acid (15.3 mg, 19.7%) as a light yellow solid. MS: m/z: Calc'd for C₁₅H₁₈N₄O₃ [M+H]⁺303, found [M+H]⁺303. ¹H NMR (400 MHz, Methanol-d4) δ 8.18 (s, 1H), 7.87 (d, J=7.7 Hz, 2H), 7.43 (d, J=7.8 Hz, 2H), 5.13 (s, 1H), 4.39 (d, J=4.3 Hz, 1H), 4.29 (d, J=8.6 Hz, 1H), 3.64 (dd, J=12.8, 4.4 Hz, 1H), 3.28-3.17 (m, 2H), 3.10 (dd, J=14.3, 8.8 Hz, 1H), 2.21 (s, 3H). Column: XBridge Prep OBD C18 Column, 30*150 mm, 5 μm; Mobile Phase A: Water(0.05% TFA), Mobile Phase B: ACN; Flow rate: 60 mL/min mL/min; Gradient: 14% B to 44% B in 10 min; Wave Length: 254 nm/220 nm nm; RT1(min): 8.68

### Synthetic Procedure of V186822

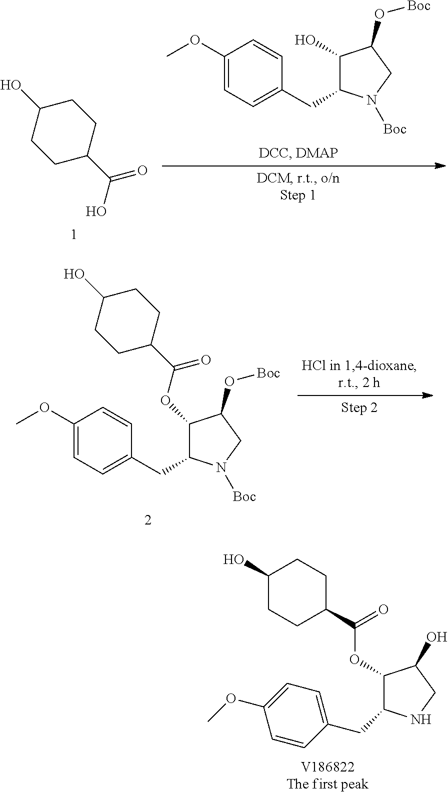 [SMILES: O=C(O)C1CCC(O)CC1 ; COc1ccc(C[C@@H]2[C@H](O)[C@@H](OC(=O)OC(C)(C)C)CN2C(=O)OC(C)(C)C)cc1 ; COc1ccc(C[C@@H]2[C@H](OC(=O)C3CCC(O)CC3)[C@@H](OC(=O)OC(C)(C)C)CN2C(=O)OC(C)(C)C)cc1 ; COc1ccc(C[C@H]2NC[C@H](O)[C@H]2OC(=O)[C@H]2CC[C@@H](O)CC2)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (200.0 mg, 0.47 mmol, 1.0 eq.) and 4-hydroxycyclohexane-1-carboxylic acid (102.1 mg, 0.71 mmol, 1.5 eq.) in DCM (10.0 mL) were added DCC (97.4 mg, 0.47 mmol, 1.0 eq.) and DMAP (86.5 mg, 0.71 mmol, 1.5 eq.) at room temperature. The resulting mixture was stirred at room temperature for overnight. LCMS showed completion of starting material. The resulting mixture was concentrated under reduced pressure. The residue was purified by reversed-phase flash chromatography with the following conditions: column, C18 silica gel; mobile phase, MeCN in Water (0.1% FA), 10% to 100% gradient in 20 min; detector, UV 254 nm, RT: 10 min) to afford tert-butyl (2R,3S,4S)-4-[(tert-butoxy carbonyl)oxy]-3-(4-hydroxycyclohexanecarbonyloxy)-2-[(4-methoxyphenyl)methyl]pyrrolidine-1-carboxylate (200.0 mg, 77.0%) as a colorless oil. MS: m/z: Calc'd for C₂₉H₄₃NO₉, [M+H−56−56]⁺438; Found 438.

Step 2: To a solution of tert-butyl (2R,3S,4S)-4-[(tert-butoxycarbonyl)oxy]-3-(4-hydroxycyclohexanecarbonyloxy)-2-[(4-methoxyphenyl)methyl]pyrrolidine-1-carboxylate (200.0 mg, 0.36 mmol, 1.0 eq.) in 1,4-dioxane (5.0 mL) was added HCl-dioxane (1M, 3 mL) at 0° C., and the resulting mixture was stirred for 2 h. LCMS showed completion of starting material. The crude product (100.0 mg) was purified by Prep-HPLC to afford The first peak compound: (2R,3S,4S)-4-hydroxy-2-[(4-methoxyphenyl)methyl]pyrrolidin-3-yl (1 s,4 s)-4-hydroxy cyclohexane-1-carboxylate (35.3 mg, 27.3%) MS: m/z: Calc'd for C₁₉H₂₇NO₅ [M+H]⁺350, Found 350 [M+H]⁺. ¹H NMR (400 MHz, Methanol-d₄) δ 7.26-7.20 (m, 2H), 6.99-6.91 (m, 2H), 5.12 (d, J=3.4 Hz, 1H), 4.33 (d, J=4.3 Hz, 1H), 4.21-4.38 (m, 1H), 3.92-3.85 (m, 1H), 3.80 (s, 3H), 3.58 (dd, J=12.8, 4.4 Hz, 1H), 3.23 (d, J=12.7 Hz, 1H), 3.10 (dd, J=14.3, 6.8 Hz, 1H), 2.96 (dd, J=14.3, 8.6 Hz, 1H), 2.59-2.54 (m, 1H), 2.04-2.01 (m, 2H), 1.75-1.65 (m, 6H). Prep-HPLC conditions: Column: Column: 254 nm/220 nm; Mobile Phase A: Xselect CSH Prep C18 Column, 30*150 mm, 5 μm, Mobile Phase B: Water (0.05% TFA); Gradient: isocratic 60 mL/min; Wave Length: 254 nm/220 nm nm; RT1(min): 2% B to 25% B in 10 min.

### Synthetic Procedure of V186823

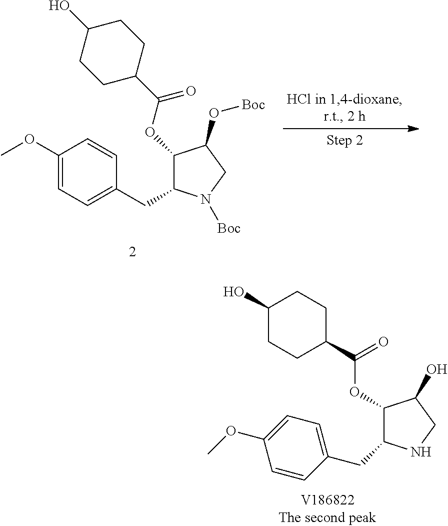 [SMILES: COc1ccc(C[C@@H]2[C@H](OC(=O)C3CCC(O)CC3)[C@@H](OC(=O)OC(C)(C)C)CN2C(=O)OC(C)(C)C)cc1 ; COc1ccc(C[C@H]2NC[C@H](O)[C@H]2OC(=O)[C@H]2CC[C@@H](O)CC2)cc1]

To a solution of tert-butyl (2R,3S,4S)-4-[(tert-butoxycarbonyl)oxy]-3-(4-hydroxycyclohexanecarbonyloxy)-2-[(4-methoxyphenyl)methyl]pyrrolidine-1-carboxylate (200.0 mg, 0.36 mmol, 1.0 eq.) in 1,4-dioxane (5.0 mL) was added HCl-dioxane (1M, 3 mL) at 0° C., and the resulting mixture was stirred for 2 h. LCMS showed completion of starting material. The crude product (100.0 mg) was purified by Prep-HPLC to afford the second peak compound: (2R,3S,4S)-4-hydroxy-2-[(4-methoxyphenyl)methyl]pyrrolidin-3-yl (1 s,4 s)-4-hydroxy cyclohexane-1-carboxylate (20.70 mg, 16.28%) as a light yellow solid. MS: m/z: Calc'd for C₁₉H₂₇NO₅ [M+H]⁺350, Found 350. The second peak: ¹H NMR (400 MHz, Methanol-d₄) (7.27-7.19 (m, 2H), 6.99-6.89 (m, 2H), 5.12 (d, J=3.4 Hz, 1H), 4.34 (d, J=4.3 Hz, 1H), 4.21-4.37 (m, 1H), 3.94-3.84 (m, 1H), 3.80 (s, 3H), 3.58 (dd, J=12.7, 4.4 Hz, 1H), 3.24 (d, J=12.7 Hz, 1H), 3.10 (dd, J=14.3, 6.9 Hz, 1H), 2.96 (dd, J=14.3, 8.6 Hz, 1H), 2.60-2.55 (m, 1H), 2.11-1.94 (m, 2H), 1.86-1.61 (m, 6H). Prep-HPLC conditions: Column: Column: 254 nm/220 nm; Mobile Phase A: Xselect CSH Prep C18 Column, 30*150 mm, 5 μm, Mobile Phase B: Water (0.05% TFA); Gradient: isocratic 60 mL/min; Wave Length: 254 nm/220 nm nm; RT1(min): 2% B to 25% B in 10 min.

### Synthetic Procedure of V186824

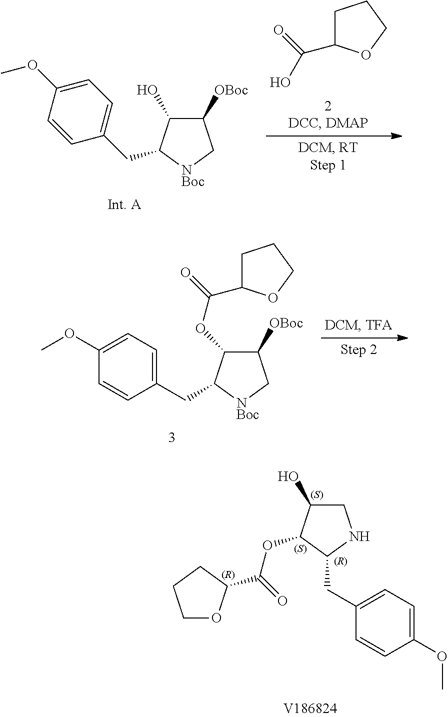

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and tetrahydro-2-furoic acid (54.8 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-(oxolane-2-carbonyloxy)pyrrolidine-1-carboxylate (94 mg, 76.3%) as a white solid. MS: m/z: Calc'd for C₂₇H₃₉NO₉ [M+H]⁺522; Found 522.

Step 2: To a stirred mixture of tert-butyl (2R,3S,4S)-4-[(tert-butoxycarbonyl)oxy]-2-[(4-methoxyphenyl)methyl]-3-(oxolane-2-carbonyloxy)pyrrolidine-1-carboxylate (95 mg, 0.18 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl (2R)-oxolane-2-carboxylate; trifluoroacetic acid (18.7 mg, 23.5%) as a yellow solid. MS: m/z: Calc'd for C₁₇H₂₃NO₅ [M+H]⁺322; Found 322. ¹H NMR (400 MHz, Methanol-d₄) δ 7.27-7.19 (m, 2H), 6.98-6.89 (m, 2H), 5.12 (d, J=3.3 Hz, 1H), 4.63 (dd, J=8.4, 5.3 Hz, 1H), 4.36 (d, J=4.4 Hz, 1H), 4.22-4.10 (m, 1H), 4.07-3.92 (m, 2H), 3.80 (d, J=0.9 Hz, 3H), 3.61 (dd, J=12.8, 4.5 Hz, 1H), 3.24 (d, J=12.8 Hz, 1H), 3.10 (dd, J=14.2, 6.9 Hz, 1H), 2.98 (dd, J=14.3, 8.7 Hz, 1H), 2.41-2.27 (m, 1H), 2.17-2.05 (m, 1H), 2.09-1.95 (m, 2H). Prep-HPLC-conditions: Column: Xselect CSH Prep Fluoro-Phenyl Column, 19*250 mm, 5 μm; Mobile Phase A: Water (0.05% TFA), Mobile Phase B: ACN; Flow rate: 25 mL/min mL/min; Gradient: 2% B to 24% B in 10 min; Wave Length: 254 nm/220 nm nm; RT1(min): 9.47

### Synthetic Procedure of V186825

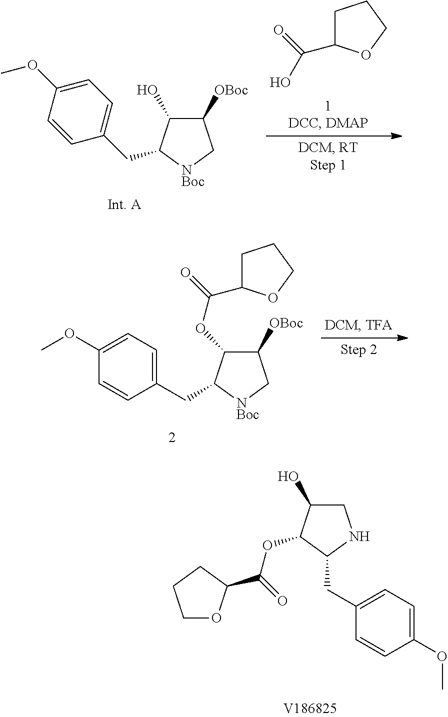

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and tetrahydro-2-furoic acid (54.8 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-(oxolane-2-carbonyloxy)pyrrolidine-1-carboxylate (94 mg, 76.3%) as a white solid. MS: m/z: Calc'd for C₂₇H₃₉NO₉ [M+H]⁺522; Found 522.

Step 2: To a stirred mixture of tert-butyl (2R,3S,4S)-4-[(tert-butoxycarbonyl)oxy]-2-[(4-methoxyphenyl)methyl]-3-(oxolane-2-carbonyloxy)pyrrolidine-1-carboxylate (95 mg, 0.18 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl (2S)-oxolane-2-carboxylate; trifluoroacetic acid (11.0 mg, 13.8%) as a white solid. MS: m/z: Calc'd for C₁₇H₂₃NO₅ [M+H]⁺322; Found 322. ¹H NMR (Methanol-d₄) δ 7.19-7.17 (m, 2H), 6.95-6.87 (m, 2H), 4.88 (d, J=3.6 Hz, 1H), 4.21 (d, J=3.6 Hz, 1H), 4.04 (dd, J=7.8, 3.6 Hz, 1H), 4.02 (s, 4H), 3.94 (s, 3H), 3.77 (d, J=17.6 Hz, 1H), 3.42-3.40 (m, 2H), 3.13 (dd, J=16.0, 7.8 Hz, 2H), 2.61-2.54 (m, 1H), 2.52-2.46 (m, 1H), 2.49-2.41 (m, 2H).

Prep-HPLC-conditions: Column: Xselect CSH Prep Fluoro-Phenyl Column, 19*250 mm, 5 μm; Mobile Phase A: Water (0.05% TFA), Mobile Phase B: ACN; Flow rate: 25 mL/min mL/min; Gradient: 2% B to 24% B in 10 min; Wave Length: 254 nm/220 nm nm; RT1(min): 9.47

### Synthetic Procedure of V186849

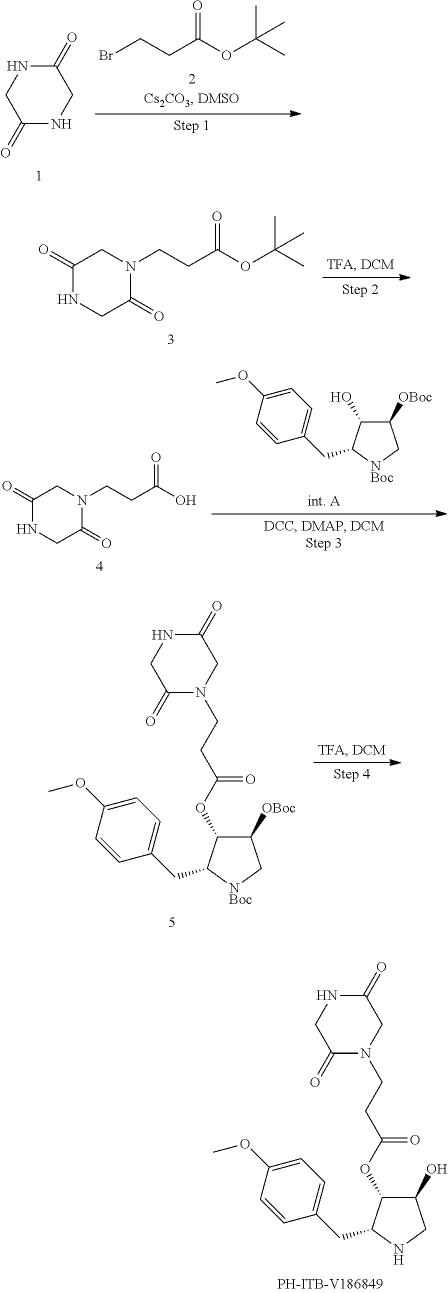

Step 1: To a solution of 2,5-piperazinedione (500 mg, 4.38 mmol, 1 eq.) in DMSO (15 mL) were added tert-butyl 3-bromopropanoate (916.2 mg, 4.38 mmol, 1 eq.) and Cs₂CO₃ (4283.2 mg, 13.14 mmol, 3 eq.) at room temperature. The mixture was stirred overnight at room temperature.

Upon completion, the reaction mixture was concentrated in vacuo. The resulting residue was dissolved with DMSO and purified by a reversed-phase column chromatography (0.05% TFA in water and acetonitrile) to afford tert-butyl 3-(2,5-dioxopiperazin-1-yl) propanoate (200 mg, 18.8%) as a white solid. MS: m/z: Calc'd for C₁₁H₁₈N₂O₄[M−H]⁻241; Found 241.

Step 2: To a solution of tert-butyl 3-(2,5-dioxopiperazin-1-yl)propanoate (200 mg, 0.82 mmol, 1 eq.) in DCM (15 mL) was added TFA (5 mL). The mixture was stirred for 2 hours at room temperature. Upon completion, the reaction mixture was concentrated in vacuo to afford 3-(2,5-dioxopiperazin-1-yl)propanoic acid which was used directly in the next step without further purification.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1.00 eq.) and 3-(2,5-dioxopiperazin-1-yl)propanoic acid (70.3 mg, 0.37 mmol, 2 eq.) in DCM (5 mL) were added DCC (116.9 mg, 0.56 mmol, 3 eq.) and DMAP (34.6 mg, 0.28 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-{[3-(2,5-dioxopiperazin-1-yl)propanoyl]oxy}-2-[(4-methoxyphenyl)methyl]pyrrolidine-1-carboxylate (100 mg, 89.4%) as a yellow oil. MS: m/z: Calc'd for C₂₉H₄₁N₃O₁₀ [M+Na]⁺614; Found 614.

Step 4: To a stirred mixture of tert-butyl (2R,3S,4S)-4-[(tert-butoxycarbonyl)oxy]-3-{[3-(2,5-dioxopiperazin-1-yl)propanoyl]oxy}-2-[(4-methoxyphenyl)methyl]pyrrolidine-1-carboxylate (70 mg, 0.11 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(2,5-dioxopiperazin-1-yl)propanoate(15 mg, 24.6%) as a white solid. MS: m/z: Calc'd for C₁₉H₂₅N₃O₆ [M+H]⁺392; Found 392. ¹H NMR (400 MHz, DMSO-d₆) δ 7.23-7.21 (m, 2H), 6.98-6.91 (m, 2H), 5.11 (d, J=3.6 Hz, 1H), 4.40 (d, J=4.2 Hz, 1H), 4.21-4.19 (m, 1H), 4.13 (d, J=2.8 Hz, 2H), 3.96 (s, 2H), 3.80 (d, J=2.8 Hz, 4H), 3.69-3.59 (m, 2H), 3.22 (d, J=12.7 Hz, 1H), 3.10 (t, J=7.4 Hz, 1H), 2.98-2.96 (m, 1H), 2.83-2.81 (m, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length:254 nm/220 nm nm; RT1(min): 8.68

### Synthetic Procedure of V186850

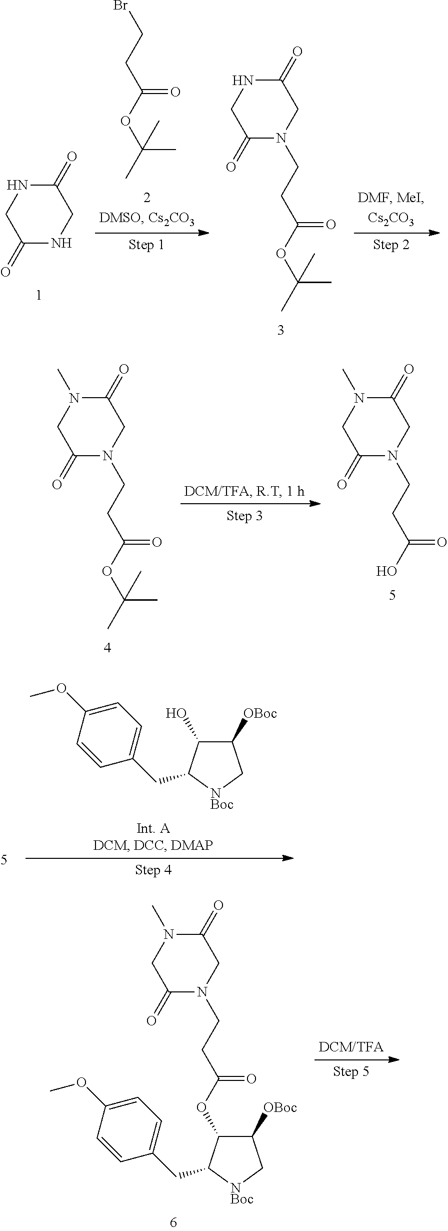

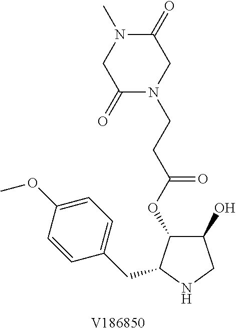 [SMILES: COc1ccc(C[C@H]2NC[C@H](O)[C@H]2OC(=O)CCN2CC(=O)N(C)CC2=O)cc1]

Step 1: To a stirred solution of 2,5-piperazinedione (1000 mg, 8.76 mmol, 1 eq.) and tert-butyl 3-bromopropanoate (733 mg, 3.51 mmol, 0.4 eq.) in DMSO (10 mL) was added Cs₂CO₃ (8566 mg, 26.29 mmol, 3 eq.). The resulting reaction mixture was stirred at room temperature for overnight. After completion of the reaction monitored by LCMS, the mixture was purified directly by reversed-phase column chromatography (10 mmol/L NH₄HCO₃ in water and acetonitrile) to afford tert-butyl 3-(2,5-dioxopiperazin-1-yl)propanoate (110 mg, 5.2%) as a white solid. MS: m/z: Calc'd for C₁₁H₁₈N₂O₄ [M−H]⁻241; Found, 241.

Step 2: To a stirred mixture of tert-butyl 3-(2,5-dioxopiperazin-1-yl)propanoate (100 mg, 0.41 mmol, 1 eq.) and Cs₂CO₃ (403 mg, 1.24 mmol, 3 eq.) in DMF (3 mL) was added Mel (117 mg, 0.83 mmol, 2 eq.) dropwise at room temperature. The resulting mixture was stirred at room temperature for 2 h. After completion of the reaction monitored by LCMS, the resulting mixture was filtered, the filter cake was washed with MeCN (3×5 mL). The filtrate was concentrated under reduced pressure. The residue was purified directly by reversed-phase column chromatography (10 mmol/L NH₄HCO₃ in water and acetonitrile) to afford tert-butyl 3-(4-methyl-2,5-dioxopiperazin-1-yl)propanoate (30 mg, 28.3%) as a white solid. MS: m/z: Calc'd for C₁₂H₂₀N₂O₄ [M+H−56]⁺201; Found, 201.

Step 3: To a stirred mixture of tert-butyl 3-(4-methyl-2,5-dioxopiperazin-1-yl)propanoate (60 mg, 0.23 mmol) in DCM (10 mL) was added TFA (2 mL) dropwise at 0° C. The resulting mixture was stirred at room temperature for 3 h. After completion of the reaction monitored by LCMS, The resulting mixture was concentrated under reduced pressure to afford 3-(4-methyl-2,5-dioxopiperazin-1-yl)propanoic acid (46 mg, 98.1%) as a light yellow oil. MS: m/z: Calc'd for C₈H₁₂N₂O₄ [M+H]⁺201; Found, 201.

Step 4: To a stirred mixture of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1 eq.) and DCC (58 mg, 0.28 mmol, 1.5 eq.) in DCM (5 mL) were added DMAP (23 mg, 0.19 mmol, 1 eq.) and 3-(4-methyl-2,5-dioxopiperazin-1-yl)propanoic acid (45 mg, 0.23 mmol, 1.2 eq.) at room temperature. The resulting mixture was stirred for at room temperature 2 days. After completion of the reaction monitored by LCMS, the resulting mixture was concentrated under reduced pressure. The residue was purified directly by reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-{[3-(4-methyl-2,5-dioxopiperazin-1-yl)propanoyl]oxy}pyrrolidine-1-carboxylate (65 mg, 56.8%) as a white solid. MS: m/z: Calc'd for C₃₀H₄₃N₃O₁₀ [M+H−56−100]⁺450; Found, 450.

Step 5: To a stirred solution of tert-butyl (2R,3S,4S)-4-[(tert-butoxycarbonyl) oxy]-2-[(4-methoxyphenyl) methyl]-3-{[3-(4-methyl-2,5-dioxopiperazin-1-yl) propanoyl]oxy}pyrrolidine-1-carboxylate (62 mg, 0.10 mmol) in DCM (5 mL) was added TFA (5 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 3-(4-methyl-2,5-dioxopiperazin-1-yl) propanoate (13.0 mg, 30.70%) as a white solid. MS: m/z: Calc'd for C₂₀H₂₇N₃O₆[M+H]⁺406; Found, 406. ¹H NMR (400 MHz, DMSO-d6) δ 7.08 (d, J=8.2 Hz, 2H), 6.81 (d, J=8.2 Hz, 2H), 6.12 (s, 1H), 5.13 (s, 1H), 4.65 (s, 1H), 4.12 (s, 1H), 3.99 (s, 2H), 3.93 (s, 2H), 3.78 (s, 1H), 3.71 (s, 2H), 3.54 (t, J=7.2 Hz, 2H), 2.80 (d, J=5.0 Hz, 3H), 2.70 (s, 4H), 2.66 (t, J=7.0 Hz, 2H), 2.58 (d, J=7.2 Hz, 1H). Prep-HPLC conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min mL/min; Gradient: 14% B to 44% B in 10 min; Wave Length: 254 nm/220 nm nm; RT1(min): 8.5.

### Synthetic Procedure of V186854

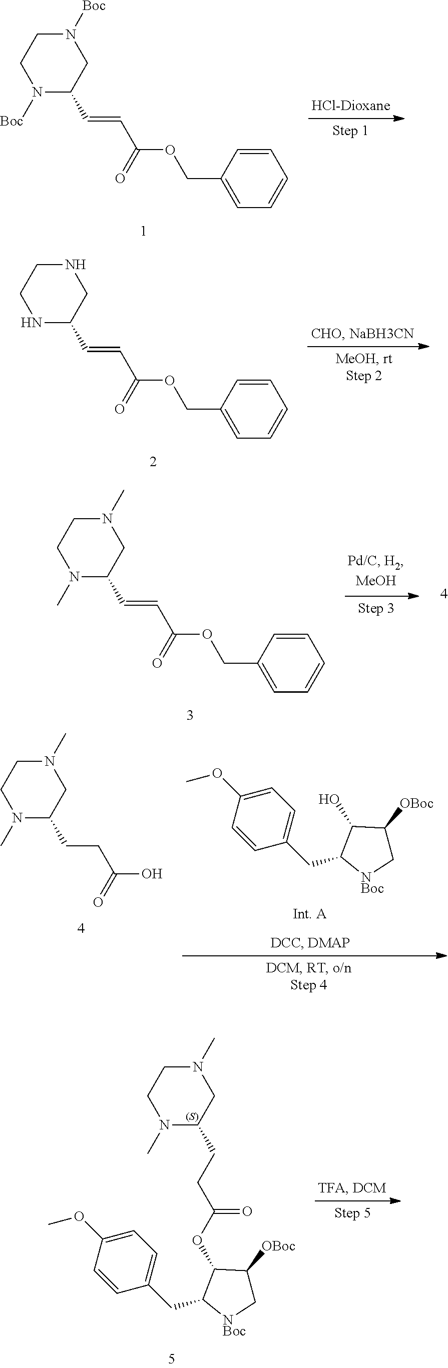

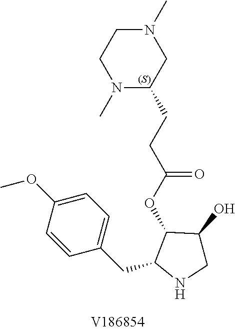

Step 1: To a stirred solution of 1,4-di-tert-butyl (2S)-2-[3-(benzyloxy)-3-oxoprop-1-en-1-yl]piperazine-1,4-dicarboxylate (200 mg, 0.45 mmol, 1.0 eq.) in dioxane (4 mL) was added HCl (gas) in 1,4-dioxane (2 mL) at room temperature. The resulting mixture was stirred at ambient temperature for 1 h. The resulting mixture was concentrated under reduced pressure. This resulted in benzyl 3-[(2S)-piperazin-2-yl]prop-2-enoate (110 mg, 97.3%) as a white solid. MS: m/z: Calc'd for C₁₄H₁₈N₂O₂ [M+H]⁺247, found 247.

Step 2: To a stirred solution of benzyl 3-[(2S)-piperazin-2-yl]prop-2-enoate (110 mg, 0.61 mmol, 1.0 eq.) and (HCHO)n (201.2 mg, 3.05 mmol, 5.0 eq.) in MeOH (3 mL) was added NaBH₃CN (56.1 mg, 1.22 mmol, 2.0 eq.) in portions at 0° C. The resulting reaction mixture was stirred at ambient temperature for 2 h. Desired product could be detected by LCMS. The reaction was quenched by the addition of Water (1 mL) at 0° C. The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in benzyl 3-[(2S)-1,4-dimethylpiperazin-2-yl]prop-2-enoate (80 mg, 47.9%) as a light yellow oil. MS: m/z: Calc'd for C₁₆H₂₂N₂O₂ [M+H]⁺275, found 275.

Step 3: Under a nitrogen atmosphere, Pd(OH)₂/C (69.9 mg, 0.50 mmol, 2.0 eq.) was added to a solution of benzyl 3-[(2S)-1,4-dimethylpiperazin-2-yl]prop-2-enoate (70 mg, 0.26 mmol, 1.0 eq.) in MeOH (3 mL), H₂ was subsequently introduced into the reaction system, and the resulting mixture was stirred at r.t. for 2 h. Upon completion, the reaction mixture was filtered and concentrated in vacuo to afford crude product 3-[(2S)-1,4-dimethylpiperazin-2-yl]propanoic acid (45 mg, 94.7%) as a light yellow oil. MS: m/z: Calc'd for C₉H₁₈N₂O₂ [M+H]⁺187, found 187.

Step 4: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and 3-[(2S)-1,4-dimethylpiperazin-2-yl]propanoic acid (45 mg, 0.26 mmol, 1 eq.) in DCM (5 mL) were added DCC (58.5 mg, 0.28 mmol, 1.5 eq.) and DMAP (23.1 mg, 0.19 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-({3-[(2S)-1,4-dimethylpiperazin-2-yl]propanoyl}oxy)-2-[(4-methoxyphenyl)methyl]pyrrolidine-1-carboxylate (50 mg, 44.7%) as a light yellow oil. MS: m/z: Calc'd for C₃₁H₄₉N₃O₈ [M+H]⁺592, found 592.

Step 5: To a stirred mixture of tert-butyl (2R,3S,4S)-4-[(tert-butoxycarbonyl)oxy]-3-({3-[(2S)-1,4-dimethylpiperazin-2-yl]propanoyl}oxy)-2-[(4-methoxyphenyl)methyl]pyrrolidine-1-carboxylate (45 mg, 0.08 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-[(2S)-1,4-dimethylpiperazin-2-yl]propanoate; trifluoroacetic acid (15.3 mg, 39.5%) as a white solid. MS: m/z: Calc'd for C₂₁H₃₃N₃O₄[M+H]⁺392, found 392. ¹H NMR (400 MHz, Methanol-d₄) δ 7.28-7.20 (m, 2H), 6.97-6.90 (m, 2H), 5.17-5.11 (m, 1H), 4.40 (dd, J=4.0, 1.6 Hz, 1H), 4.24-4.20 (m, 1H), 3.80 (s, 3H), 3.64-3.60 (m, 1H), 3.53-3.38 (m, 3H), 3.22 (d, J=12.7 Hz, 1H), 3.15-3.05 (m, 3H), 3.04-2.90 (m, 3H), 2.82 (d, J=2.7 Hz, 3H), 2.72 (s, 3H), 2.66-2.54 (m, 1H), 2.23-2.15 (m, 1H), 2.01-1.93 (m, 1H). Prep-HPLC-conditions: Column: XBridge BEH Prep OBD Amide C18 Column, 19*250 mm, 5 μm; Mobile Phase A: Water (0.1% TFA), Mobile Phase B: ACN; Flow rate: 25 mL/min mL/min; Gradient: 95% B to 70% B in 8 min; Wave Length: 254 nm/220 nm nm; RT1(min): 6.65

### Synthetic Procedure of V186857

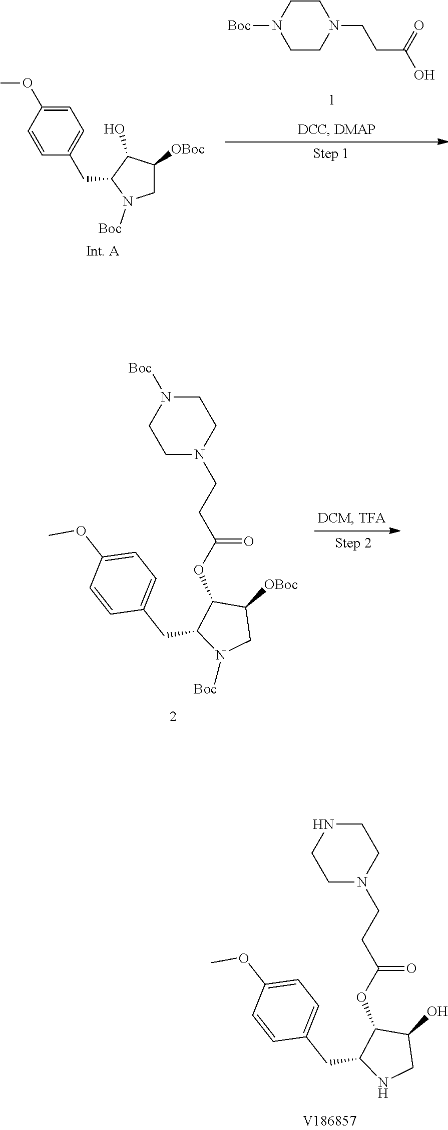

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and 3-[4-(tert-butoxycarbonyl)piperazin-1-yl]propanoic acid (97.5 mg, 0.37 mmol, 2 eq.) in DCM (5 mL) were added DCC (58.4 mg, 0.28 mmol, 1.5 eq.) and DMAP (23.08 mg, 0.18 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl 4-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)piperazine-1-carboxylate (60 mg, 47.85%) as a white solid. MS: m/z: Calc'd for C₃₄H₅₃N₃O₁₀ [M+H]⁺665, found 665.

Step 2: To a stirred mixture of tert-butyl 4-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)piperazine-1-carboxylate (60 mg, 0.09 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(piperazin-1-yl)propanoate (19.9 mg, 58.2%) as a white solid. MS: m/z: Calc'd for C₁₉H₂₉N₃O₄ [M+H]⁺364; Found, 364. ¹H NMR (400 MHz, DMSO-d₆) δ 7.11 (d, J=8.1 Hz, 2H), 6.81 (d, J=8.1 Hz, 2H), 4.65 (d, J=3.8 Hz, 1H), 3.92-3.90 (m, 1H), 3.70 (s, 3H), 3.29 (s, 1H), 3.16 (m, 3H), 2.72-2.51 (m, 10H), 2.31 (s, 3H).

Prep-HPLC-conditions: Column: XSelect CSH Prep C18 OBD Column, 5 μm, 30*150 mm; Mobile Phase A: Water (0.05% TFA), Mobile Phase B: ACN; Flow rate:60 mL/min; Gradient:5 B to 35 B in 33 min; 254/220 nm; RT1:6.3; RT2:; Injection Volumn: ml; Number Of Runs:;

### Synthetic Procedure of V186858

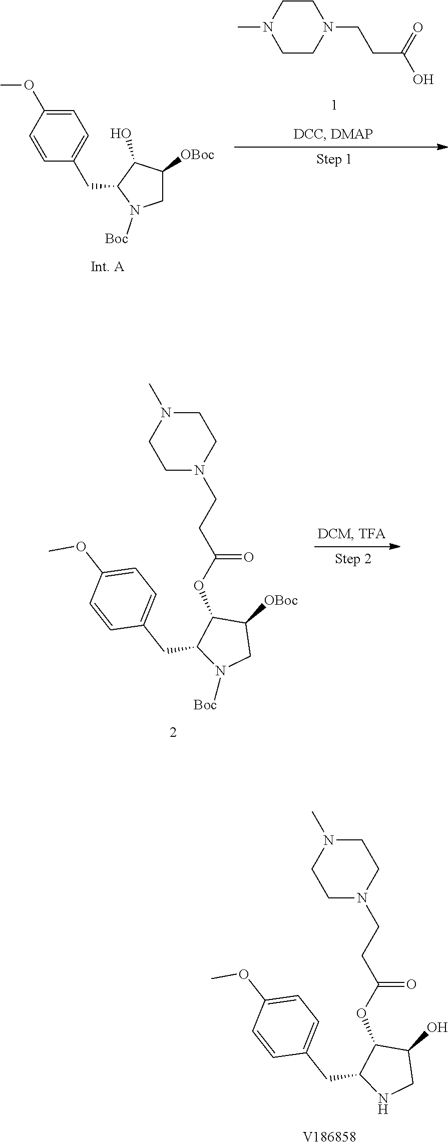

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-(4-methylpiperazin-1-yl)propanoic acid (81.3 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxy carbonyl)oxy]-2-[(4-methoxyphenyl)methyl]-3-{[3-(4-methylpiperazin-1-yl)propanoyl]oxy}pyrrolidine-1-carboxylate (60 mg, 43.98%) as a white solid. MS: m/z: Calc'd for C₃₀H₄₇N₃O₈ [M+H]⁺578, found 578.

Step 2: To a stirred mixture of tert-butyl (2R,3S,4S)-4-[(tert-butoxycarbonyl)oxy]-2-[(4-methoxyphenyl)methyl]-3-{[3-(4-methylpiperazin-1-yl)propanoyl]oxy}pyrrolidine-1-carboxylate (60 mg, 0.10 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(4-methylpiperazin-1-yl)propanoate; trifluoroacetic acid (16.8 mg, 32.3%) as a white solid. MS: m/z: Calc'd for C₂₂H₃₂F₃N₃O₆[M+H]⁺378; Found, 378. ¹H NMR (400 MHz, DMSO-d₆) δ 7.29-7.21 (m, 2H), 6.97-6.91 (m, 2H), 5.15 (d, J=3.4 Hz, 1H), 4.38 (d, J=4.3 Hz, 1H), 4.19-4.17 (m, 1H), 3.80 (s, 3H), 3.67-3.57 (m, 1H), 3.28 (s, 2H), 3.23 (d, J=12.7 Hz, 3H), 3.11 (m, 1H), 3.04-2.84 (m, 10H), 2.78-2.76 (m, 2H). Prep-HPLC-conditions: Column: XSelect CSH Prep C18 OBD Column, 5 μm, 30*150 mm; Mobile Phase A: Water (0.05% TFA), Mobile Phase B:ACN; Flow rate:60 mL/min; Gradient:2 B to 11 B in 39 min; 254/220 nm; RT1:8.68; RT2:; Injection Volumn: ml; Number Of Runs:;

### Synthetic Procedure of V186859

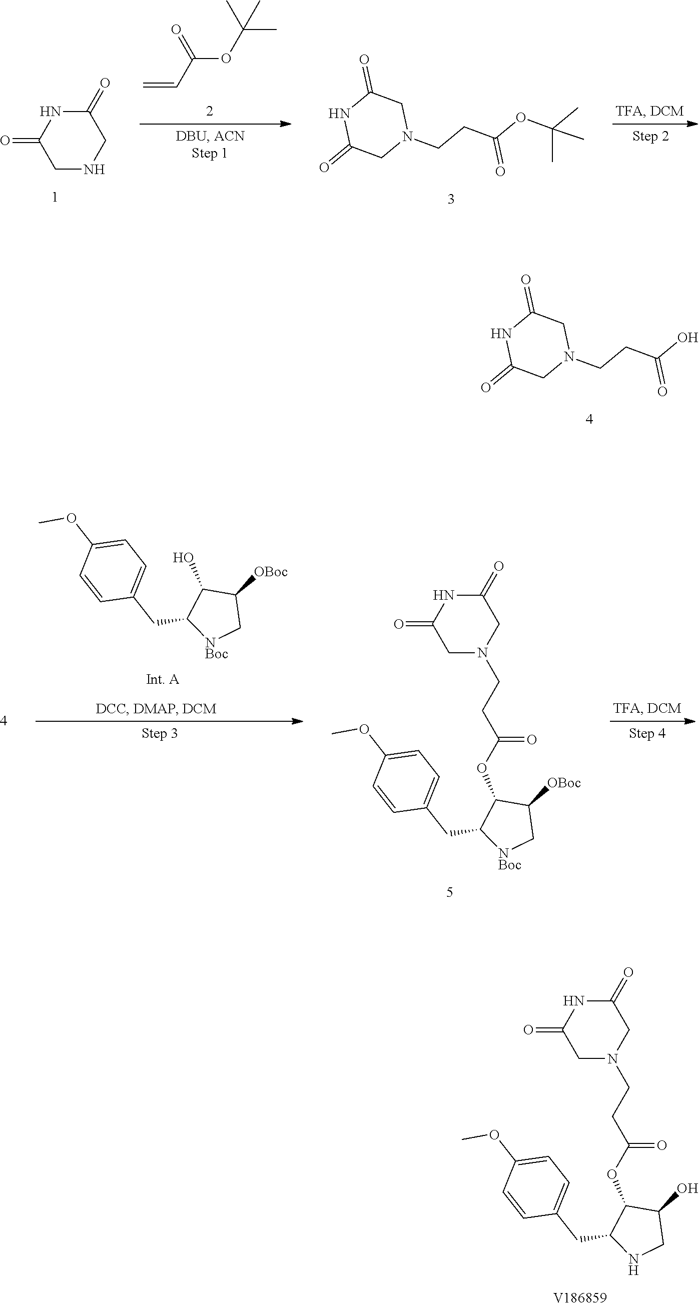

Step 1: To a stirred solution of piperazine-2,6-dione (300 mg, 2.63 mmol, 1.0 eq.) and tert-butyl prop-2-enoate (303.29 mg, 2.37 mmol, 0.9 eq.) in ACN (5 mL) was added DBU (800.5 mg, 5.26 mmol, 2.0 eq.) at room temperature. The resulting reaction mixture was stirred at ambient temperature for overnight. The resulting mixture was extracted with EtOAc (3×5 mL). The combined organic layers were washed with water (2×1 mL), dried over anhydrous Na₂SO₄. After filtration, the filtrate was concentrated under reduced pressure. The crude product was used in the next step directly without further purification.

Step 2: To a stirred solution of tert-butyl 3-(3,5-dioxopiperazin-1-yl)propanoate (300 mg, 1.24 mmol, 1.0 eq.) in DCM (4 mL) was added TFA (2 mL) at room temperature. The resulting reaction mixture was stirred at ambient temperature for 1 h. The resulting mixture was concentrated under reduced pressure. The crude product was used in the next step directly without further purification.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.24 mmol, 1.0 eq.) and 3-(3,5-dioxopiperazin-1-yl)propanoic acid (87.9 mg, 0.47 mmol, 2.0 eq.) in DCM (5 mL) were added DCC (73.1 mg, 0.35 mmol, 1.5 eq.) and DMAP (28.9 mg, 0.24 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-{[3-(3,5-dioxopiperazin-1-yl)propanoyl]oxy}-2-[(4-methoxyphenyl)methyl]pyrrolidine-1-carboxylate (60 mg, 43.0%) as a white solid. MS: m/z: Calc'd for C₂₉H₄₁N₃O₁₀ [M+H]⁺480, found 480.

Step 4: To a stirred mixture of tert-butyl (2R,3S,4S)-4-[(tert-butoxycarbonyl)oxy]-3-{[3-(3,5-dioxopiperazin-1-yl)propanoyl]oxy}-2-[(4-methoxyphenyl)methyl]pyrrolidine-1-carboxylate (55 mg, 0.09 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(3,5-dioxopiperazin-1-yl)propanoate; trifluoroacetic acid (23.4 mg, 49.1%) as a white solid. MS: m/z: Calc'd for C₁₉H₂₅N₃O₆ [M+H]⁺392, found 392. ¹H NMR (400 MHz, Methanol-d₄) δ 7.22 (d, J=8.1 Hz, 2H), 6.92 (dd, J=8.7, 2.4 Hz, 2H), 5.13 (d, J=3.4 Hz, 1H), 4.38-4.30 (m, 1H), 4.22-4.12 (m, 1H), 3.79 (d, J=1.8 Hz, 3H), 3.64-3.56 (m, 1H), 3.45 (d, J=2.1 Hz, 4H), 3.23 (d, J=12.6 Hz, 1H), 3.08 (dd, J=14.2, 7.2 Hz, 1H), 3.02-2.85 (m, 3H), 2.80-2.74 (m, 2H). Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 2% B to 23% B in 10 min; Wave Length: 254 nm/220 nm nm; RT1(min): 8.68

### Synthetic Procedure of V186862

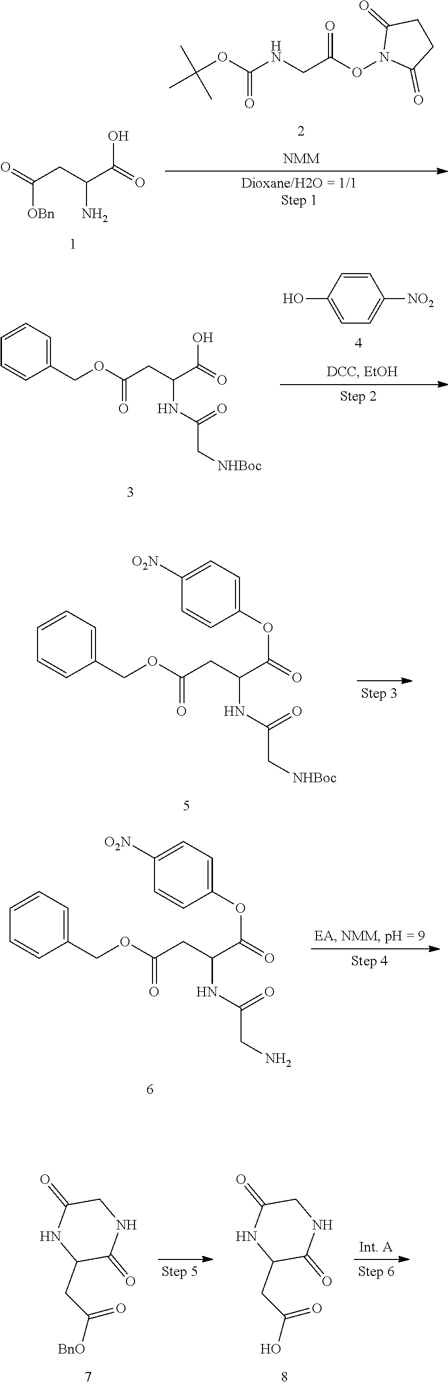

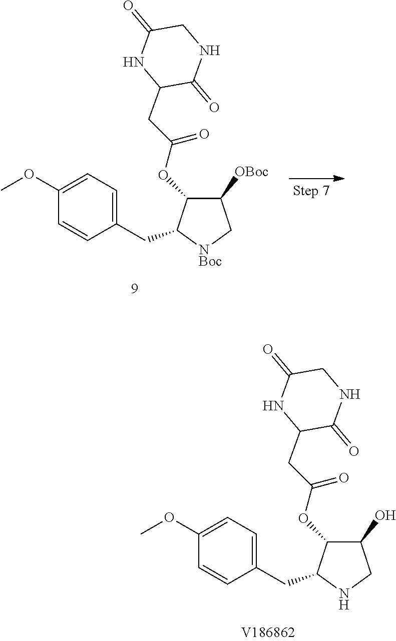

Step 1: To a stirred solution of 2-amino-4-(benzyloxy)-4-oxobutanoic acid (2 g, 8.95 mmol, 1 eq.) in dioxane (10 mL) and H₂O (10 mL), was added 2,5-dioxopyrrolidin-1-yl 2-[(tert-butoxycarbonyl)amino]acetate (2.44 g, 8.95 mmol, 1 eq.) and NMM (1.0 g, 9.85 mmol, 1.1 eq.). The resulting mixture was stirred for 2 h at room temperature. Upon completion, the reaction mixture was concentrated in vacuo. The residue was purified by Prep-TLC to obtain 4-(benzyloxy)-2-{2-[(tert-butoxycarbonyl)amino]acetamido}-4-oxobutanoic acid (2 g, 58.6%) as a white solid. MS: m/z: Calc'd for C₁₈H₂₄N₂O₇ [M+H]⁺381, found 381.

Step 2: To a stirred solution of 4-(benzyloxy)-2-{2-[(tert-butoxycarbonyl)amino]acetamido}-4-oxobutanoic acid (2 g, 5.25 mmol, 1 eq.) in EtOH (30 mL) were added P-nitrophenol (877.6 mg, 6.31 mmol, 1.2 eq.) and DCC (1301.8 mg, 6.310 mmol, 1.2 equiv), the mixture was stirred at room temperature for 2 h. Desired product could be detected by LCMS. Upon completion, the reaction mixture was concentrated in vacuo. The residue was purified by Prep-TLC to obtain 1-benzyl 4-nitrophenyl 3-{2-[(tert-butoxycarbonyl)amino]acetamido}butanedioate (2.8 g, 106.19%). MS: m/z: Calc'd for C₂₄H₂₇N₃O₉[M+H−56]⁺446, found 446.

Step 3: To a stirred solution of 1-benzyl 4-nitrophenyl 3-{2-[(tert-butoxycarbonyl)amino]acetamido}butanedioate (2.8 g, 5.58 mmol) in DCM (10 mL) was added TFA (2 mL). The mixture was stirred at room temperature for 2 h. Upon completion, the reaction mixture was concentrated in vacuo to obtain crude product 1-benzyl 4-nitrophenyl 3-(2-aminoacetamido) butanedioate (2 g, 89.2%) as a white solid. MS: m/z: Calc'd for C₁₉H₁₉N₃O₇ [M+H]⁺402, found 402.

Step 4: To a stirred solution of 1-benzyl 4-nitrophenyl 3-(2-aminoacetamido)butanedioate (2 g, 4.98 mmol, 1 eq.) in EtOH (20 mL) was added NMM (0.55 g, 5.48 mmol, 1.1 eq.). The mixture was stirred at room temperature for 2 h. Upon completion, the mixture was basified to pH=9 with 2 M HCl. The resulting mixture was concentrated under reduced pressure to obtain crude product as a white solid which was used directly in the next step without further purification. MS: m/z: Calc'd for C₁₃H₁₄N₂O₄ [M+H]⁺263, found 263.

Step 5: Under a nitrogen atmosphere, Pd/C (100 mg, 2 eq.) was added to a solution of benzyl 2-(3,6-dioxopiperazin-2-yl)acetate (6.1 mg, 0.02 mmol, 1 eq.) in THF (10 mL), H₂ was subsequently introduced into the reaction system, and the resulting mixture was stirred at r.t. for 2 h. Upon completion, the reaction mixture was filtered and concentrated in vacuo to afford crude product which was used directly in the next step without further purification. MS: m/z: Calc'd for C₆H₈N₂O₄[M−H]⁻173, found 173.

Step 6: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and (3,6-dioxopiperazin-2-yl)acetic acid (81.2 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford (2R,3S,4S)-4-hydroxy-2-[(4-methoxyphenyl) methyl]pyrrolidin-3-yl 2-(3,6-dioxopiperazin-2-yl)acetate (50 mg, 56.1%) as a white solid. MS: m/z: Calc'd for C₁₈H₂₃N₃O₆ [M−H]⁻576, found 576.

Step 7: To a stirred mixture of tert-butyl (2R,3S,4S)-4-[(tert-butoxycarbonyl)oxy]-3-{[2-(3,6-dioxopiperazin-2-yl)acetyl]oxy}-2-[(4-methoxyphenyl)methyl]pyrrolidine-1-carboxylate (60 mg, 0.10 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(3,6-dioxopiperazin-2-yl)acetate (3.2 mg, 8.02%) as a white solid. MS: m/z: Calc'd for C₁₈H₂₃N₃O₆ [M+H]⁺378; Found, 378. ¹H NMR (400 MHz, Methanol-d₄) δ 7.26-7.20 (m, 2H), 6.98-6.91 (m, 2H), 5.12 (s, 1H), 4.46-4.38 (m, 2H), 4.19-4.17 (m, 1H), 3.99 (t, J=1.4 Hz, 2H), 3.80 (d, J=0.9 Hz, 3H), 3.61 (dd, J=12.7, 4.9 Hz, 1H), 3.22 (d, J=12.7 Hz, 1H), 3.11-3.04 (m, 2H), 3.03-2.89 (m, 2H). Prep-HPLC-conditions: Column: XSelect CSH Prep C18 OBD Column, 5 μm, 30*150 mm; Mobile Phase A:Water (0.05% TFA), Mobile Phase B:ACN; Flow rate:60 mL/min; Gradient:30 B to 2 B in 30 min; 254/220 nm; RT1:18; RT2:; Injection Volumn: ml; Number Of Runs:;

### Synthetic Procedure of V186864

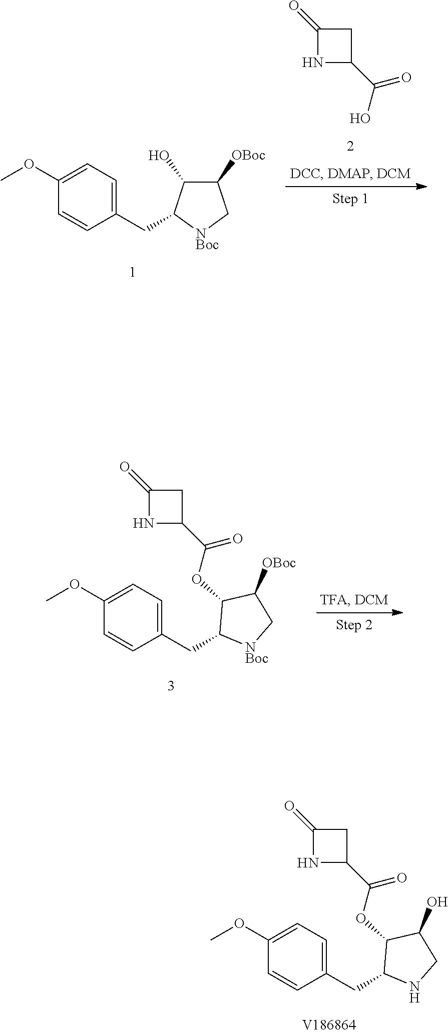

Step 1: To the mixture solution of tert-butyl (2R,3S,4S)-4-tert-butoxycarbonyloxy-3-hydroxy-2-[(4-methoxyphenyl)methyl]pyrrolidine-1-carboxylate (180. mg, 0.4300 mmol) and 4-oxoazetidine-2-carboxylic acid (73.37 mg, 0.6400 mmol) in DCM (10 mL) was added DCC (131.54 mg, 0.6400 mmol), DMAP (77.89 mg, 0.6400 mmol) at 0° C., the mixture solution was stirred at room temperature overnight. LCMS show the reaction was completed and the reaction was purified by TLC(PE:EA=1:1) to afford tert-butyl (2R,3S,4S)-4-tert-butoxycarbonyloxy-2-[(4-methoxyphenyl)methyl]-3-(4-oxoazetidine-2-carbonyl)oxy-pyrrolidine-1-carboxylate (75 mg, 0.1441 mmol, 33.897% yield) as white semi-solid.

MS: m/z: Calc'd for C₂₆H₃₆N₂O₉ [M−56+22H]⁺487; Found, 487.

Step 2: To the mixture solution of tert-butyl (2R,3S,4S)-4-[(tert-butoxycarbonyl)oxy]-2-[(4-methoxyphenyl)methyl]-3-(4-oxoazetidine-2-carbonyloxy)pyrrolidine-1-carboxylate (55 mg, 0.106 mmol, 1 equiv) in DCM (5 mL, 78.653 mmol, 744.46 equiv) was added TFA (1 mL, 13.463 mmol). The mixture solution was stirred at room temperature for 3 h, LCMS show the reaction was completed. Concentrated and purified by prep-HPLC to afford (2R,3S,4S)-4-hydroxy-2-[(4-methoxyphenyl)methyl]pyrrolidin-3-yl 4-oxoazetidine-2-carboxylate; trifluoroacetic acid (27.2 mg, 58.62%) as off-white solid. MS: m/z: Calc'd for C₁₆H₂₀N₂O₅ [M+H]⁺321; Found, 321. ¹H NMR (400 MHz, DMSO-d₆) (7.28-7.19 (m, 2H), 6.97-6.87 (m, 2H), 5.00-4.91 (m, 1H), 4.36-4.21 (m, 2H), 4.11-4.00 (m, 1H), 3.78-3.71 (m, 3H), 3.52-3.44 (m, 1H), 3.35-3.23 (m, 1H), 3.13-2.84 (m, 4H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 28% B in 10 min; Wave Length: 254 nm/220 nm nm; RT1(min): 8.68

### Synthetic Procedure of V186865

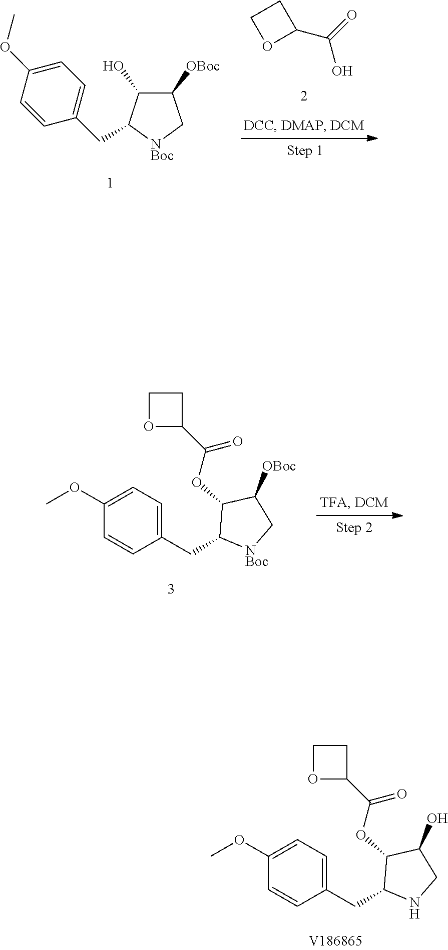

Step 1: To a stirred solution of oxetane-2-carboxylic acid (48.2 mg, 0.47 mmol, 2 eq.) and a tert-butyl(2R,3S,4S)-4-[(tert-butoxycarbonyl)oxy]-3-hydroxy-2-[(4-methoxyphenyl) methyl]pyrrolidine-1-carboxylate (100 mg, 0.24 mmol, 1 eq.) in DCM (5 mL) was added DCC (73.1 mg, 0.35 mmol, 1.5 eq.) and DMAP (28.9 mg, 0.24 mmol, 1 eq.) at 0° C. under nitrogen atmosphere. The resulting mixture was stirred at 0° C. for 2 h. Upon completion, the crude product was purified by reversed phase flash chromatography (10 mmol/L NH₄HCO₃ in water and acetonitrile) to afford tert-butyl (2R,3S,4S)-4-[(tert-butoxycarbonyl)oxy]-2-[(4-methoxyphenyl) methyl]-3-(oxetane-2-carbonyloxy)pyrrolidine-1-carboxylate (110 mg, 91.7%) as a white solid. MS: m/z: Calc'd for C₂₆H₃₇NO₉ [M+H−56−56]⁺396, found 396.

Step 2: To a stirred solution of tert-butyl (2R,3S,4S)-4-[(tert-butoxycarbonyl)oxy]-2-[(4-methoxyphenyl)methyl]-3-(oxetane-2-carbonyloxy)pyrrolidine-1-carboxylate (100 mg, 0.16 mmol, 1 eq.) in DCM (3 mL) was added TFA (1 mL) dropwise at 0° C. under nitrogen atmosphere. The resulting mixture was stirred for 1 h at room temperature. Upon completion, the reaction mixture was concentrated in vacuo. The resulting residue was dissolved with DMSO and purified by prep-HPLC to afford (2R,3S,4S)-4-hydroxy-2-(4-methoxybenzyl)pyrrolidin-3-yl oxetane-2-carboxylate FA salt (18 mg, 24.9%) as a white solid. MS: m/z: Calc'd for C₁₆H₂₁NO₅ [M+H]⁺308, found [M+H]⁺308. ¹H NMR (400 MHz, DMSO-d6) δ 8.21 (s, 1H), 7.15 (dd, J=8.2, 5.2 Hz, 2H), 6.94-6.83 (m, 2H), 5.27-5.11 (m, 1H), 4.81 (d, J=4.0 Hz, 1H), 4.69-4.53 (m, 2H), 4.08 (d, J=5.1 Hz, 1H), 3.72 (s, 3H), 3.65 (s, 1H), 3.28 (m, 1H), 3.08-2.88 (m, 1H), 2.79-2.59 (m, 4H). Prep-HPLC-conditions: Column: UV 254 nm/220 nm XselectCSH Prep OBD C18 Column, 30*150 mm, 5 μm; Mobile Phase A: Water (0.1% FA). Mobile Phase B: ACN 60 mL/min; Gradient: 2% B to 25% B in 8 min 6.43. Wave Length: 254 nm/220 nm nm; RT1(min): 6.2

### Synthetic Procedure of V186866

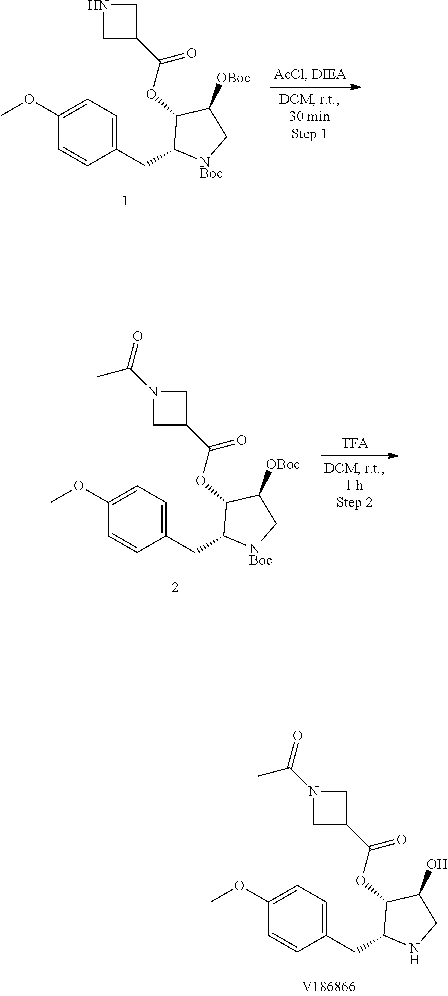

Step 1: To a stirred solution of tert-butyl (2R,3S,4S)-3-(azetidine-3-carbonyloxy)-4-[(tert-butoxycarbonyl)oxy]-2-[(4-methoxyphenyl)methyl]pyrrolidine-1-carboxylate (120.0 mg, 0.24 mmol, 1.0 eq.) and DIEA (61.2 mg, 0.24 mmol, 2.0 eq.) in DCM (5.0 mL) was added acetyl chloride (55.7 mg, 0.72 mmol, 3.0 eq.) at 0° C. The resulting mixture was stirred for 30 min at room temperature. LCMS showed completion of starting material. The reaction mixture was concentrated under reduced pressure. The residue was purified by reversed-phase flash chromatography with the following conditions: column, C18 silica gel; mobile phase, MeCN in Water (0.1% FA), 10% to 100% gradient in 20 min; detector, UV 220 nm, to afford tert-butyl (2R,3S,4S)-3-(1-acetylazetidine-3-carbonyloxy)-4-[(tert-butoxycarbonyl)oxy]-2-[(4-methoxyphenyl)methyl]pyrrolidine-1-carboxylate (125.0 mg, 94.9%) as a white solid. MS: m/z: Calc'd for C₂₈H₄₀N₂O₉, [M+H−56−56]⁺437; Found 437.

Step 2: To a solution of tert-butyl (2R,3S,4S)-3-(1-acetylazetidine-3-carbonyloxy)-4-[(tert-butoxycarbonyl)oxy]-2-[(4-methoxyphenyl)methyl]pyrrolidine-1-carboxylate (125.0 mg, 0.23 mmol, 1.0 equiv) in DCM (8.0 mL) was stirred TFA (4.0 mL) for 1 h at room temperature. LCMS showed completion of starting material. The resulting mixture was concentrated under reduced pressure. The crude product (150.0 mg) was purified by Prep-HPLC to afford (2R,3S,4S)-4-hydroxy-2-[(4-methoxyphenyl)methyl]pyrrolidin-3-yl 1-acetylazetidine-3-carboxylate (23.3 mg, 29.0%) as a yellow solid. MS: m/z: Calc'd for C₁₈H₂₄N₂O₅ [M+H]⁺349, Found 349. ¹H NMR (400 MHz, Methanol-d₄) δ 7.27-7.19 (m, 2H), 6.98-6.90 (m, 2H), 5.17 (d, J=3.4 Hz, 1H), 4.53-4.35 (m, 3H), 4.33-4.19 (m, 2H), 4.14-4.10 (m, 1H), 3.80 (s, 3H), 3.74-3.56 (m, 2H), 3.24 (d, J=12.7 Hz, 1H), 3.09 (d, J=6.6 Hz, 1H), 2.98-2.95 (m, 1H), 1.91 (d, J=1.2 Hz, 3H). Prep-HPLC conditions: Column: Atlantis T3 Prep OBD Column, 19*250 mm, 5 μm; Mobile Phase A: Water (0.05% TFA), Mobile Phase B: ACN; Flow rate: 25 mL/min; Gradient: 5% B to 27% B in 11 min; Wave Length: 254 nm/220 nm nm; RT1(min): 11.07.

### Synthetic Procedure of V186868

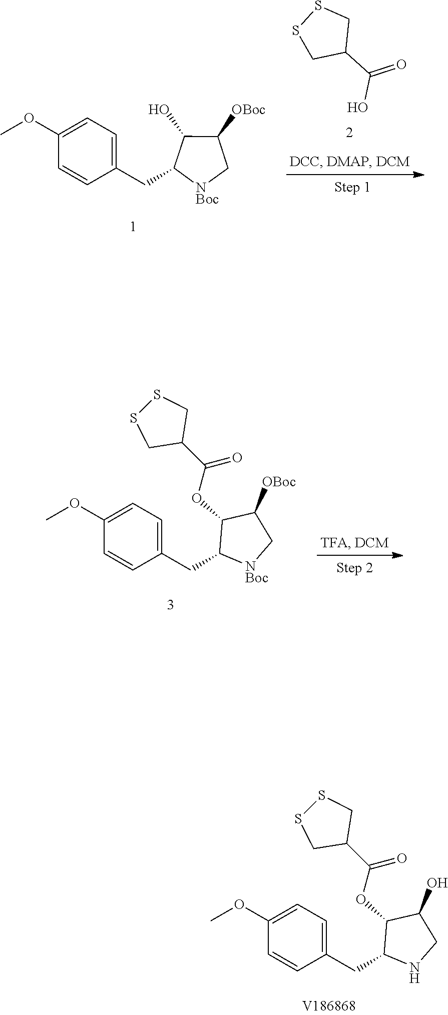

Step 1: To the mixture solution of tert-butyl (2R,3S,4S)-4-tert-butoxycarbonyloxy-3-hydroxy-2-[(4-methoxyphenyl)methyl]pyrrolidine-1-carboxylate (180 mg, 0.43 mmol) and dithiolane-4-carboxylic acid (95 mg, 0.64 mmol) in DCM (10 mL) was added DCC (131 mg, 0.64 mmol), DMAP (77 mg, 0.64 mmol) at 0° C., the mixture solution was stirred at room temperature overnight. LCMS show the reaction was completed and the reaction was purified by TLC (PE:EA=1:1) to afford tert-butyl (2R,3S,4S)-4-tert-butoxycarbonyloxy-3-(dithiolane-4-carbonyloxy)-2-[(4-methoxyphenyl)methyl]pyrrolidine-1-carboxylate (75 mg, 0.13 mmol, 31.7% yield) as white semi-solid. MS: m/z: Calc'd for C₂₆H₃₇N₈S₂ [M+22H]⁺578; Found, 578.

Step 2: To the mixture solution of tert-butyl (2R,3S,4S)-4-[(tert-butoxycarbonyl)oxy]-3-(1,2-dithiolane-4-carbonyloxy)-2-[(4-methoxyphenyl)methyl]pyrrolidine-1-carboxylate (70 mg, 0.12 mmol, 1 equiv) in DCM (5 mL) was added TFA (1 mL). The mixture solution was stirred at room temperature for 3 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl 1,2-dithiolane-4-carboxylate; trifluoroacetic acid (20.9 mg) as an off-white solid. MS: m/z: Calc'd for C₁₆H₂₁NO₄S₂[M+H]⁺356; Found, 356. ¹H NMR (400 MHz, DMSO-d₆) δ 7.28-7.17 (m, 2H), 6.95-6.87 (m, 2H), 4.96-4.90 (m, 1H), 4.26-4.19 (m, 1H), 4.07-3.98 (m, 1H), 3.92-3.83 (m, 1H), 3.74 (s, 3H), 3.69-3.59 (m, 2H), 3.53-3.45 (m, 2H), 3.33-3.28 (m, 1H), 3.12-3.06 (m, 1H), 3.03-2.86 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 32% B in 10 min; Wave Length: 254 nm/220 nm nm; RT1(min): 8.68

### Synthetic Procedure of V186870

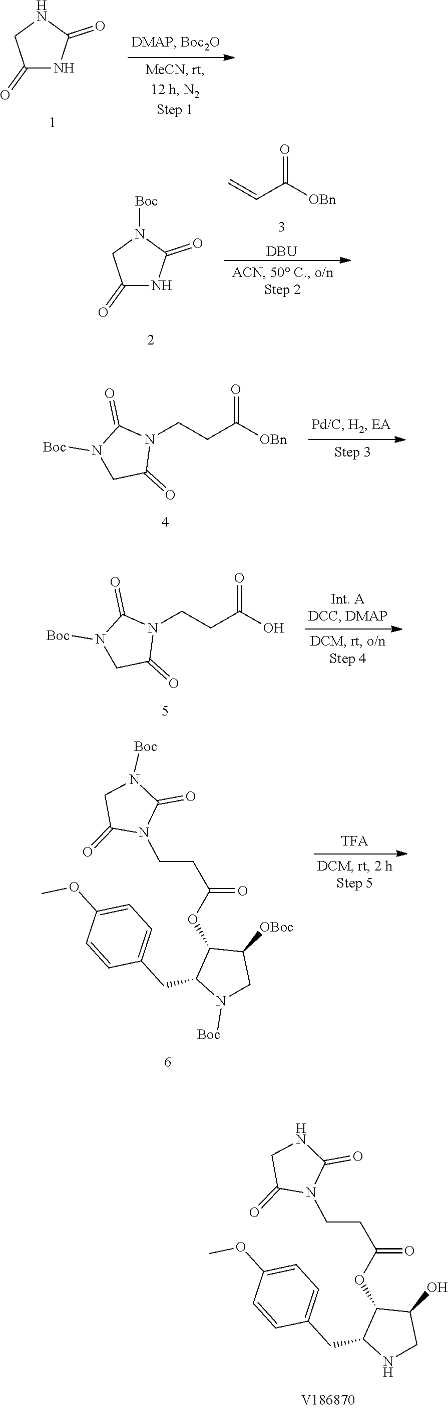

Step 1: To a stirred solution of hydantoin (1 g, 9.99 mmol, 1 eq.) in ACN (50 mL) was added DMAP (1.2 g, 9.99 mmol, 1 eq.) and (Boc)₂O (2.6 g, 11.99 mmol, 1.2 eq.) at 0° C. The reaction was stirred at room temperature for 12 h. After completion of the reaction monitored by LCMS, The reaction mixture was quenched with water and extracted with EA. The extracts were washed with brine, dried over anhydrous sodium sulfate, filtered and concentrated. The residue was purified by Prep-TLC (PE/EA 2:1) to afford tert-butyl 2,4-dioxoimidazolidine-1-carboxylate (425 mg, 21.2%) as a white solid. MS: m/z: Calc'd for C₈H₁₂N₂O₄[M−H]⁺199; Found, 199.

Step 2: To a stirred solution of tert-butyl 2,4-dioxoimidazolidine-1-carboxylate (400 mg, 1.99 mmol, 1 eq.) in ACN (5 mL) were added benzyl prop-2-enoate (648.1 mg, 3.99 mmol, 2 eq.) and DBU (608.3 mg, 3.99 mmol, 2 eq.) dropwise at 0° C. The reaction was stirred at 50° C. for overnight. After completion of the reaction monitored by LCMS, the resulting mixture was concentrated under reduced pressure. The crude product was purified by reverse phase flash with the following conditions (0.5% NH₄HCO₃ in H₂O/ACN) to afford tert-butyl 3-[3-(benzyloxy)-3-oxopropyl]-2,4-dioxoimidazolidine-1-carboxylate (138 mg, 19.0%) as a white solid. MS: m/z: Calc'd for C₁₈H₂₂N₂O₆ [M+H⁺16]⁺379; Found, 379.

Step 3: To a stirred solution of tert-butyl 3-[3-(benzyloxy)-3-oxopropyl]-2,4-dioxoimidazolidine-1-carboxylate (130 mg, 0.35 mmol, 1 eq.) in MeOH (10 mL) were added Pd/C (65 mg, 0.61 mmol, 1.70 eq.). The reaction mixture was stirred under one atmosphere of hydrogen for 12 h. The reaction was filtered through Celite and washed with MeOH (30.0 mL). The filtrate was concentrated under reduced pressure to afford 3-[3-(tert-butoxycarbonyl)-2,5-dioxoimidazolidin-1-yl]propanoic acid (130 mg, 133.1%) as a yellow solid. MS: m/z: Calc'd for C₁₁H₁₆N₂O₆ [M−H]⁻271; Found, 271.

Step 4: To a stirred solution of 3-[3-(tert-butoxycarbonyl)-2,5-dioxoimidazolidin-1-yl]propanoic acid (Int.A, 55 mg, 0.20 mmol, 1 eq.) and tert-butyl (2R,3S,4S)-4-[(tert-butoxy carbonyl)oxy]-3-hydroxy-2-[(4-methoxyphenyl)methyl]pyrrolidine-1-carboxylate (171.1 mg, 0.40 mmol, 2 eq.) in DCM (6 mL) were added DCC (62.5 mg, 0.30 mmol, 1.5 eq.) and DMAP (24.6 mg, 0.20 mmol, 1 eq.) at 0° C. The reaction was stirred at r.t. for overnight. After completion of the reaction monitored by LCMS, the resulting mixture was filtered, the filter cake was washed with DCM (1×10 mL). The filtrate was concentrated under reduced pressure, the residue was purified by Prep-HPLC to afford tert-butyl 3-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)-2,4-dioxoimidazolidine-1-carboxylate (120 mg, 87.6%) as a white solid.

MS: m/z: Calc'd for C₃₃H₄₇N₃O₁₂[M−H]⁻676; Found, 676.

Step 5: To a stirred solution of tert-butyl 3-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)-2,4-dioxoimidazolidine-1-carboxylate (36 mg, 0.05 mmol) in DCM (3 mL) was added TFA (0.6 mL) dropwise at 0° C. The reaction was stirred at r.t. for 2 h. The resulting mixture was concentrated under reduced pressure. The crude product (70 mg) was purified by Prep-HPLC to afford (2R,3S,4S)-4-hydroxy-2-[(4-methoxyphenyl)methyl]pyrrolidin-3-yl 3-(2,5-dioxoimidazolidin-1-yl)propanoate (6.3 mg) as a white solid. MS: m/z: Calc'd for C₁₈H₂₃N₃O₆ [M+H]⁺378; Found, 378. ¹H NMR (400 MHz, Methanol-d₄) δ 7.24-7.21 (m, 2H), 6.94-6.91 (m, 2H), 5.11-5.09 (m, 1H), 4.40-4.35 (m, 1H), 4.22-4.20 (m, 2H), 3.80 (s, 3H), 3.62-3.57 (m, 1H), 3.22 (d, J=12.6 Hz, 1H), 3.10-2.06 (m, 1H), 2.98-2.93 (m, 1H), 2.73-2.63 (m, 1H), 2.61-2.58 (m, 2H), 2.15-2.11 (m, 2H). Prep-HPLC-conditions: Column: XBridge Prep OBD C18 Column, 30*150 mm, 5 μm; Mobile Phase A: Water(0.05% TFA), Mobile Phase B: ACN; Flow rate: 60 mL/min mL/min; Gradient: 33% B to 63% B in 10 min; Wave Length: 254 nm/220 nm nm; RT1(min): 8.18

### Synthetic Procedure of V186871

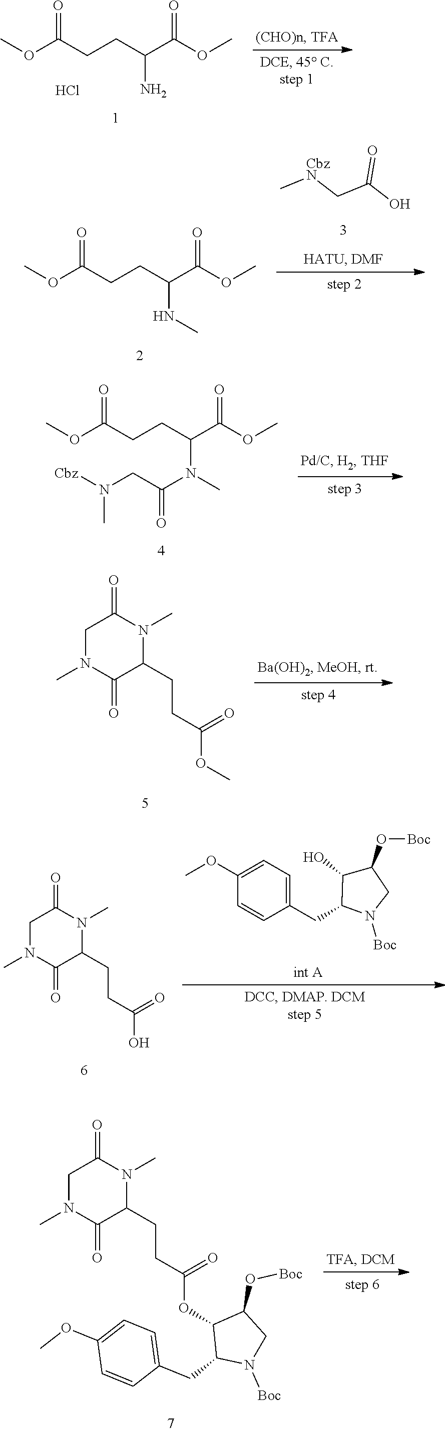

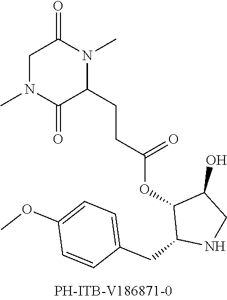 [SMILES: COc1ccc(C[C@H]2NC[C@H](O)[C@H]2OC(=O)CCC2C(=O)N(C)CC(=O)N2C)cc1]

Step 1: To a stirred solution of 1,5-dimethyl 2-aminopentanedioate (4 g, 22.83 mmol, 1 equiv) and (HCHO)n (6.17 g, 68.49 mmol, 3 equiv) in DCE (30 mL) was added TFA (5.21 g, 45.66 mmol, 2 equiv) at RT. The resulting mixture was stirred overnight at 45° C. Upon completion, the reaction mixture was concentrated in vacuo. The crude residue was purified by reversed-phase flash with the following conditions (0.05% TFA in water/ACN) to afford 1,5-dimethyl 2-(methylamino)pentanedioate (3 g, 69.4%) as a yellow oil. MS: m/z: Calc'd for C₈H₁₅NO₄ [M+H]⁺190, found 190.

Step 2: A solution of 1,5-dimethyl 2-(methylamino)pentanedioate (3 g, 15.85 mmol, 2 equiv) in DMF (20 mL) was treated with {[(benzyloxy)carbonyl](methyl)amino}acetic acid (1.77 g, 7.92 mmol, 1 equiv) at RT under air atmosphere followed by the addition of HATU (4.52 g, 11.89 mmol, 1.5 equiv) and DIEA (5.12 g, 39.63 mmol, 5 equiv). The resulting mixture was stirred for overnight at RT. Upon completion, the reaction mixture was concentrated in vacuo. The crude product was purified by reversed phase flash with the following conditions (0.05% TFA in water/ACN) to afford 1,5-dimethyl 2-(2-{[(benzyloxy)carbonyl]amino}-N-methyl acetamido)pentanedioate (700 mg, 23.21%) as a yellow oil. MS: m/z: Calc'd for C₁₉H₂₆N₂O₇ [M+H]⁺395, found 395.

Step 3: Under a nitrogen atmosphere, Pd/C (150 mg) was added to a solution of 1,5-dimethyl 2-(2-{[(benzyloxy)carbonyl]amino}-N-methylacetamido)pentanedioate (700 mg, 1.84 mmol) in THF (20 mL), H₂ was subsequently introduced into the reaction system, and the resulting mixture was stirred at r.t. for 4 h. Upon completion, the reaction mixture was filtered and concentrated in vacuo to afford crude product which was used directly in the next step without further purification. MS: m/z: Calc'd for C₁₀H₁₆N₂O₄ [M+H]⁺229, found 229.

Step 4: A solution of methyl 3-(1,4-dimethyl-3,6-dioxopiperazin-2-yl)propanoate (440 mg, 1.92 mmol, 1 equiv) in MeOH (15 mL) was treated with Ba(OH)₂ (1821.7 mg, 5.78 mmol, 3 equiv). The resulting mixture was stirred overnight at RT. Upon completion, the reaction mixture was concentrated in vacuo. The crude product was purified by Prep-HPLC with the following conditions (Column: XBridge Prep Shield RP18 OBD C18 Column, 30*150 mm, 5 μm; Mobile Phase A: Water (0.05% TFA), Mobile Phase B: ACN; Flow rate: 60 mL/min mL/min; Gradient: 0% B to 0% B in 5.5 min; Wave Length: 254 nm/220 nm nm; RT1(min): 8.18) to afford 3-(1,4-dimethyl-3,6-dioxopiperazin-2-yl)propanoic acid (200 mg, 48.4%) as a yellow oil. MS: m/z: Calc'd for C₉H₁₄N₂O₄ [M−H]⁻213, found 213.

Step 5: A solution of 3-(1,4-dimethyl-3,6-dioxopiperazin-2-yl)propanoic acid (80.9 mg, 0.37 mmol, 2 equiv) in DCM (10 mL) was treated with tert-butyl (2R,3S,4S)-4-[(tert-butoxycarbonyl) oxy]-3-hydroxy-2-[(4-methoxyphenyl)methyl]pyrrolidine-1-carboxylate (80 mg, 0.18 mmol, 1 equiv), DCC (116.9 mg, 0.56 mmol, 3 equiv) and DMAP (34.6 mg, 0.28 mmol, 1.5 equiv) at 0° C. The resulting mixture was stirred overnight at RT. Upon completion, the reaction mixture was concentrated in vacuo. The residue was purified by reversed phase flash with the following conditions (0.05% TFA/ACN) to afford tert-butyl (2R,3S,4S)-4-[(tert-butoxycarbonyl)oxy]-3-{[3-(1,4-dimethyl-3,6-dioxopiperazin-2-yl)propanoyl]oxy}-2-[(4-methoxyphenyl)methyl]pyrrolidine-1-carboxylate (93 mg, 79.4%) as a yellow solid. MS: m/z: Calc'd for C₃₁H₄₅N₃O₁₀ [M+H]⁺620, found 620.

Step 6: To a solution of tert-butyl (2R,3S,4S)-4-[(tert-butoxycarbonyl)oxy]-3-{[3-(1,4-dimethyl-3,6-dioxopiperazin-2-yl)propanoyl]oxy}-2-[(4-methoxyphenyl)methyl]pyrrolidine-1-carboxylate (85 mg, 0.13 mmol) in DCM (9 mL) was added TFA (3 mL). The mixture was stirred for 2 hours at RT. Upon completion, the reaction mixture was concentrated in vacuo. The residue was purified by Prep-HPLC to afford ((2R,3S,4S)-4-hydroxy-2-[(4-methoxyphenyl)methyl]pyrrolidin-3-yl 3-(1,4-dimethyl-3,6-dioxopiperazin-2-yl)propanoate; trifluoroacetic acid (24.3 mg, 33.14%) as a white solid. MS: m/z: Calc'd for C₂₁H₂₉N₃O₆ [M+H]⁺420, found 420. ¹H NMR (400 MHz, Methanol-d₄) δ 7.31-7.20 (m, 2H), 6.94-6.92 (m, 2H), 5.09-5.07 (m, 1H), 4.42-4.40 (m, 1H), 4.29-4.15 (m, 2H), 4.03-4.01 (m, 1H), 3.94-3.91 (m, 1H), 3.71-3.59 (m, 1H), 3.22-3.20 (m, 1H), 3.11-3.09 (m, 1H), 3.03-2.89 (m, 6H), 2.68-2.50 (m, 2H), 2.41-2.27 (m, 1H), 2.23-2.09 (m, 1H). Prep-HPLC conditions: UV 254 nm/220 nm Xselect CSH Prep C18 C18 Column, 30*150 mm, 5 μm Water (0.05% TFA) ACN 60 mL/min 2% B to 26% B in 10 min 8.68.

### Synthetic Procedure of V186873

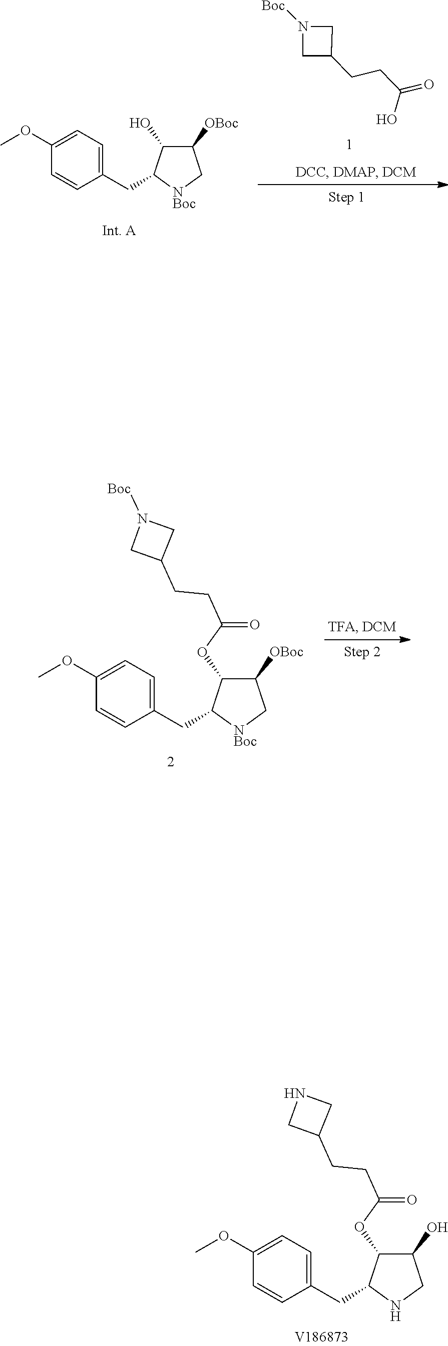

Step 1: To the mixture solution of tert-butyl (2R,3S,4S)-4-tert-butoxycarbonyloxy-3-hydroxy-2-[(4-methoxyphenyl)methyl]pyrrolidine-1-carboxylate (70 mg, 0.17 mmol) and 3-(1-tert-butoxycarbonylazetidin-3-yl)propanoic acid (56.8 mg, 0.25 mmol) in DCM (10 mL) were added DCC (51.1 mg, 0.25 mmol), DMAP (30.2 mg, 0.25 mmol) at 0° C., the mixture solution was stirred at room temperature for overnight. LCMS showed the reaction was completed and the reaction was purified by TLC (PE:EA=1:1) to afford tert-butyl (2R,3S,4S)-3-[3-(1-tert-butoxycarbonylazetidin-3-yl)propanoyloxy]-4-tert-butoxycarbonyloxy-2-[(4-methoxyphenyl)methyl]pyrrolidine-1-carboxylate (90 mg, 0.1418 mmol, 85.7% yield) as white semi-solid. MS: m/z: Calc'd for C₃₃H₅₀N₂O₁₀ [M+H−100−100]⁺436; Found, 436

Step 2: To the mixture solution of tert-butyl (2R,3S,4S)-3-({3-[1-(tert-butoxycarbonyl) azetidin-3-yl]propanoyl}oxy)-4-[(tert-butoxycarbonyl)oxy]-2-[(4-methoxyphenyl)methyl]pyrrolidine-1-carboxylate (85 mg, 0.134 mmol, 1 equiv) in DCM (10 mL) was added TFA (2 mL), the mixture solution was stirred at room temperature for 4 h. LCMS show the reaction was completed, and concentrated and purified by prep-HPLC and after freeze-drying to afford (2R,3S,4S)-4-hydroxy-2-[(4-methoxyphenyl)methyl]pyrrolidin-3-yl 3-(azetidin-3-yl)propanoate (27.1 mg, NaN) as off white solid. MS: m/z: Calc'd for C₁₈H₂₆N₂O₄ [M+H]⁺335; Found, 335.

¹H NMR (400 MHz, DMSO-d₆) δ 7.09 (d, J=8.0 Hz, 2H), 6.82 (d, J=8.0 Hz, 2H), 4.70-4.62 (m, 1H), 3.96-3.87 (m, 1H), 3.87 (s, 3H), 3.55-3.44 (m, 2H), 3.37-3.25 (m, 3H), 3.22-3.11 (m, 1H), 2.65-2.56 (m, 3H), 2.54-2.52 (m, 1H), 2.34-2.25 (m, 2H), 1.86-1.74 (m, 2H).
Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 25% B to 55% B in 8 min; Wave Length: 254 nm/220 nm nm; RT1(min): 6.98

### Synthetic Procedure of V186921

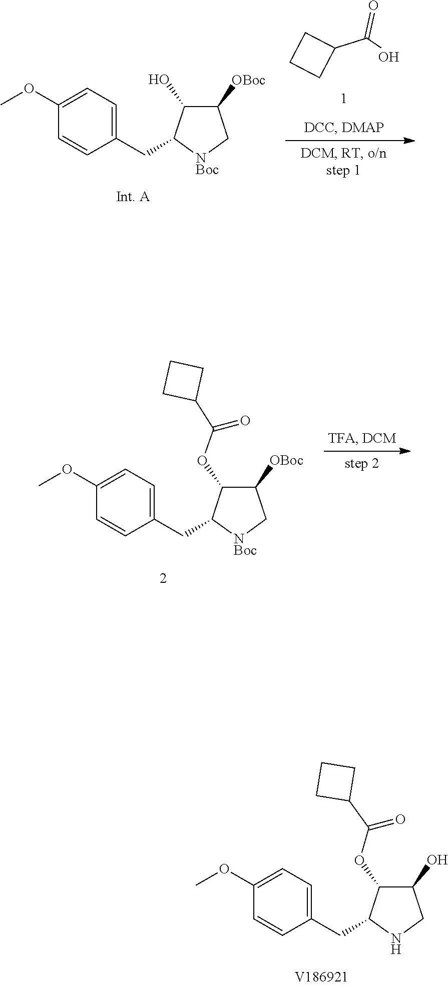

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq.) and cyclobutanecarboxylic acid (37.8 mg, 0.38 mmol, 2 eq.) in DCM (1 mL) was added DCC (58.5 mg, 0.28 mmol, 1.5 eq.) and DMAP (23.1 mg, 0.19 mmol, 1 eq.) at 0° C. The resulting reaction mixture was stirred at ambient temperature for overnight. Upon completion, the reaction mixture was concentrated in vacuo. The residue was purified by Prep-TLC (PE/EA 5:1) to afford tert-butyl (2R,3S,4S)-4-[(tert-butoxycarbonyl)oxy]-3-(cyclobutanecarbonyloxy)-2-[(4-methoxy phenyl)methyl]pyrrolidine-1-carboxylate (70 mg, 73.29%) as a light yellow oil. MS: m/z: Calc'd for C₂₇H₃₉NO₈[M+H−56−100]⁺350, found 350.

Step 2: To a stirred mixture of tert-butyl (2R,3S,4S)-4-[(tert-butoxycarbonyl)oxy]-3-(cyclobutanecarbonyloxy)-2-[(4-methoxyphenyl)methyl]pyrrolidine-1-carboxylate (65 mg, 0.13 mmol) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-ylcyclobutanecarboxylate; trifluoroacetic acid (12.8 mg, 23.5%) as a light yellow solid. MS: m/z: Calc'd for C₁₅H₁₈FNO₄ [M+H]⁺306, found 306. ¹H NMR (400 MHz, Methanol-d4) δ 7.22 (d, J=8.1 Hz, 2H), 6.94 (d, J=8.1 Hz, 2H), 5.09 (d, J=4.1 Hz, 1H), 4.35 (d, J=4.3 Hz, 1H), 4.20 (d, J=8.4 Hz, 1H), 3.80 (s, 3H), 3.60-3.55 (m, 1H), 3.38 (d, J=9.1 Hz, 1H), 3.21 (s, 1H), 3.09 (dd, J=14.3, 6.9 Hz, 1H), 2.99-2.92 (m, 1H), 2.38-2.28 (m, 4H), 2.15-2.06 (m, 1H), 1.97-1.95 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm nm; RT1(min): 8.68

### Synthetic Procedure of V186988

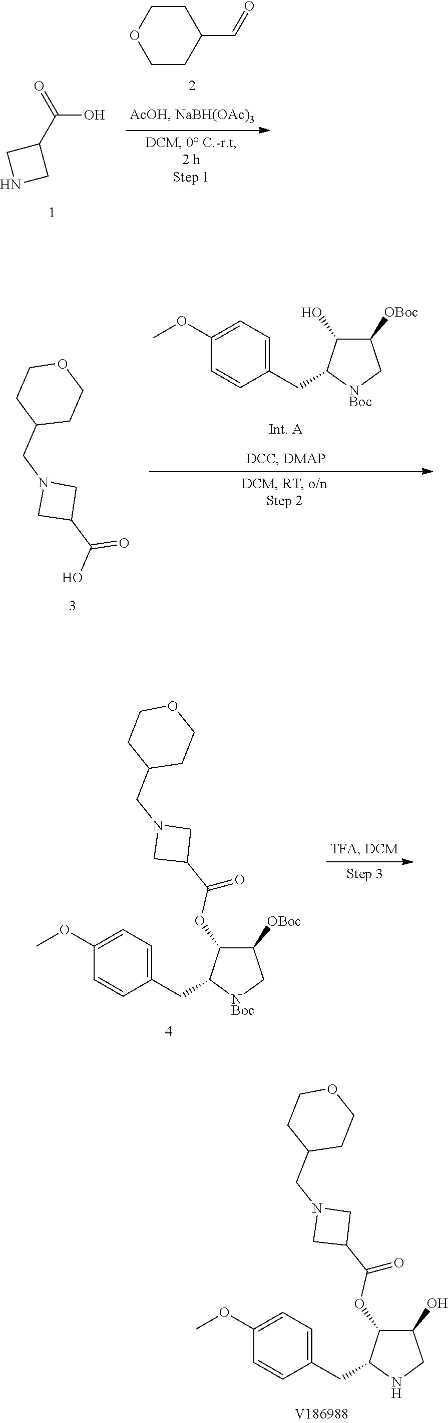

Step 1: To a stirred solution of 3-azetidinecarboxylic acid (200 mg, 1.98 mmol, 1.0 eq) and oxane-4-carbaldehyde (225.8 mg, 1.98 mmol, 1.0 eq) in DCM (10 mL) was added AcOH (237.6 mg, 3.96 mmol, 2.0 eq) at room temperature. The resulting mixture was stirred 0.5 h at 25° C. Then add NaBH(OAc)₃ (838.5 mg, 3.96 mmol, 2 eq) at 0° C. The resulting mixture was stirred at ambient temperature for 2 h. After completion of the reaction monitored by LCMS. The resulting mixture was concentrated under reduced pressure. This resulted in 1-(oxan-4-ylmethyl)azetidine-3-carboxylic acid (380 mg, 96.4%) as a light yellow oil. MS: m/z: Calc'd for C₁₀H₁₇NO₃ [M+H]⁺200, found 200.

Step 2: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and 2-fluoroprop-2-enoic acid (75.3 mg, 0.38 mmol, 2.0 eq.) in DCM (5 mL) were added DCC (58.46 mg, 0.283 mmol, 1.5 eq.) and DMAP (23.1 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-[1-(oxan-4-ylmethyl)azetidine-3-carbonyloxy]pyrrolidine-1-carboxylate (65 mg, 56.9%). MS: m/z: Calc'd for C₃₂H₄₈N₂O₉ [M+H]⁺605, found 605.

Step 3: To a stirred mixture of (2R,3S,4S)-4-[(tert-butoxycarbonyl)oxy]-2-[(4-methoxyphenyl)methyl]-3-[1-(oxan-4-ylmethyl)azetidine-3-carbonyloxy]pyrrolidine-1-carboxylate (55 mg, 0.09 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 1-(oxan-4-ylmethyl)azetidine-3-carboxylate; trifluoroacetic acid (15.7 mg, 32.4%) as a white solid. MS: m/z: Calc'd for C₁₅H₁₈FNO₄ [M+H]⁺405, found 405. ¹H NMR (400 MHz, Methanol-d4) δ 7.31-7.19 (m, 2H), 6.98-6.91 (m, 2H), 5.27-5.18 (m, 1H), 4.48 (d, J=4.4 Hz, 5H), 4.28-4.21 (m, 1H), 4.01-3.89 (m, 3H), 3.80 (s, 3H), 3.64 (dd, J=12.9, 4.5 Hz, 1H), 3.46-3.40 (m, 2H), 3.27-3.19 (m, 3H), 3.12 (dd, J=14.5, 6.0 Hz, 1H), 3.02-2.96 (m, 1H), 2.06-1.87 (m, 1H), 1.70-1.59 (m, 2H), 1.42-1.32 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm nm; RT1(min): 8.68

### Synthetic Procedure of V186989

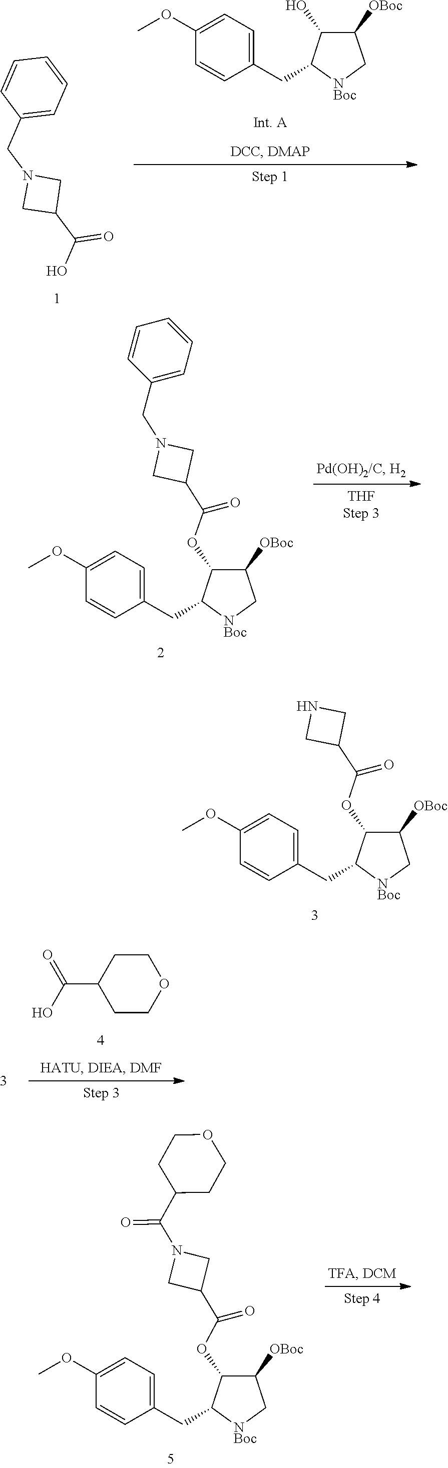

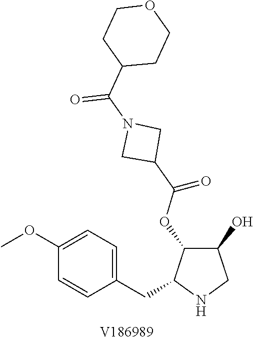 [SMILES: COc1ccc(C[C@H]2NC[C@H](O)[C@H]2OC(=O)C2CN(C(=O)C3CCOCC3)C2)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 500 mg, 1.18 mmol, 1.0 eq.) and 1-benzylazetidine-3-carboxylic acid (270.9 mg, 1.42 mmol, 1.2 eq.) in DCM (5 mL) were added DCC (365.4 mg, 1.77 mmol, 1.5 eq.) and DMAP (144.2 mg, 1.18 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-3-(1-benzylazetidine-3-carbonyloxy)-4-[(tert-butoxycarbonyl)oxy]-2-[(4-methoxyphenyl)methyl]pyrrolidine-1-carboxylate (490 mg, 69.6%) as a light yellow oil. MS: m/z: Calc'd for C₂₅H₃₄FNO₈[M+H]⁺597, found 597.

Step 2: Under a nitrogen atmosphere, Pd(OH)₂/C (400 mg) was added to a solution of tert-butyl (2R,3S,4S)-3-(1-benzylazetidine-3-carbonyloxy)-4-[(tert-butoxycarbonyl)oxy]-2-[(4-methoxyphenyl)methyl]pyrrolidine-1-carboxylate (200 mg, 0.34 mmol, 1 eq.) in THF (5 mL), H₂ was subsequently introduced into the reaction system, and the resulting mixture was stirred at r.t. for 2 h.

Upon completion, the reaction mixture was filtered and concentrated in vacuo to afford crude product tert-butyl (2R,3S,4S)-3-(azetidine-3-carbonyloxy)-4-[(tert-butoxycarbonyl)oxy]-2-[(4-methoxyphenyl)methyl]pyrrolidine-1-carboxylate (100 mg, 58.9%) as a light yellow oil. MS: m/z: Calc'd for C₂₆H₃₈N₂O₅[M+H]⁺507, found 507.

Step 3: To a stirred solution of tert-butyl (2R,3S,4S)-3-(azetidine-3-carbonyloxy)-4-[(tert-butoxycarbonyl)oxy]-2-[(4-methoxyphenyl)methyl]pyrrolidine-1-carboxylate (100 mg, 0.20 mmol, 1.0 eq.) and oxane-4-carboxylic acid (25.7 mg, 0.20 mmol, 1.0 eq.) in DMF (3 mL) was added HATU (112.6 mg, 0.30 mmol, 1.5 eq.) and DIEA (51.03 mg, 0.39 mmol, 2 eq.) at room temperature.

The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-[1-(oxane-4-carbonyl)azetidine-3-carbonyloxy]pyrrolidine-1-carboxylate (50 mg, 40.9%) as a light yellow oil.

MS: m/z: Calc'd for C₃₂H₄₆N₂O₁₀ [M+H−56−56]⁺507, found 507.

Step 4: To a stirred mixture of tert-butyl (2R,3S,4S)-4-[(tert-butoxycarbonyl)oxy]-2-[(4-methoxyphenyl)methyl]-3-[1-(oxane-4-carbonyl)azetidine-3-carbonyloxy]pyrrolidine-1-carboxylate (50 mg, 0.09 mmol, 1 eq.) in DCM (3 mL) was added TFA (1 mL) at 0° C. under nitrogen atmosphere.

The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 1-(oxane-4-carbonyl)azetidine-3-carboxylate; trifluoroacetic acid (16.0 mg, 31.83%) as a yellow solid. MS: m/z: Calc'd for C₂₂H₃₀N₂O₆[M+H]⁺419, found 419. ¹H NMR (400 MHz, Methanol-d₄) δ 7.28-7.20 (m, 2H), 6.95 (d, J=8.2 Hz, 2H), 5.22-5.14 (m, 1H), 4.57-4.51 (m, 1H), 4.45 (dd, J=12.0, 5.4 Hz, 2H), 4.25 (dd, J=12.4, 8.3 Hz, 2H), 4.16-4.10 (m, 1H), 4.02-3.94 (m, 2H), 3.80 (s, 3H), 3.72-3.66 (m, 1H), 3.62-3.58 (m, 1H), 3.48 (dd, J=13.2, 9.8 Hz, 2H), 3.24 (d, J=12.7 Hz, 1H), 3.12 (dd, J=14.7, 6.6 Hz, 1H), 3.00-2.94 (m, 1H), 2.66-2.54 (m, 1H), 1.81-1.71 (m, 2H), 1.63 (d, J=13.4 Hz, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.68

### Synthetic Procedure of V186990

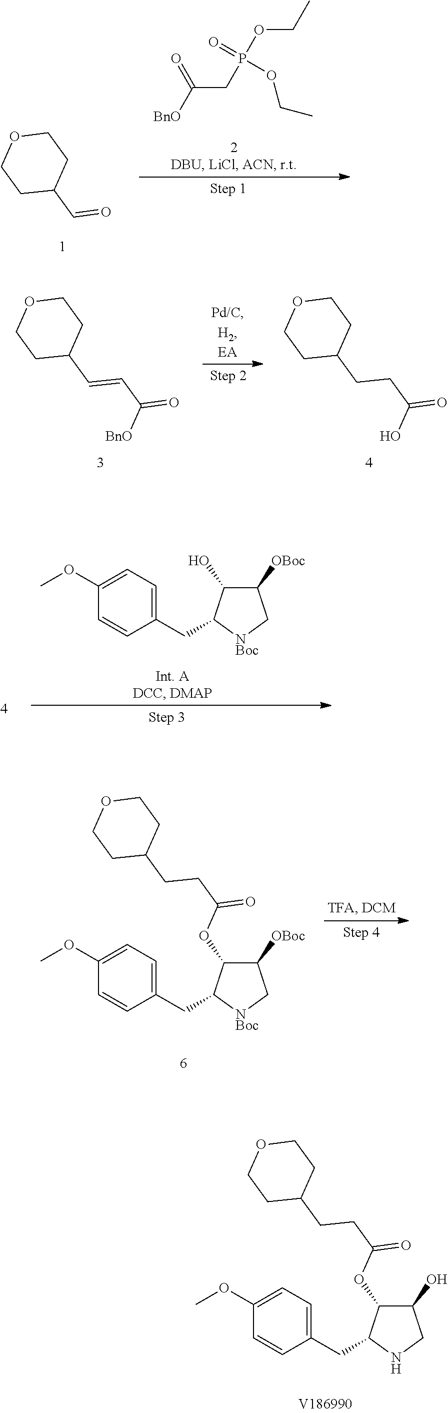

Step 1: To a solution of oxane-4-carbaldehyde (200 mg, 1.75 mmol, 1 eq) in ACN (10 mL) were added DBU (320 mg, 2.10 mmol, 1.2 eq) and LiCl (104 mg, 2.45 mmol, 1.4 eq) at 0° C. under nitrogen atmosphere. The mixture was stirred for 30 min, followed by the addition of benzyl 2-(diethoxyphosphoryl)acetate (1003 mg, 3.50 mmol, 2 eq). The solution was stirred at room temperature for 4 h. After completion of reaction monitored by TLC. The mixture was concentrated and the residue was purified by Prep-TLC (PE/EA 1:1) to afford benzyl (2E)-3-(oxan-4-yl)prop-2-enoate (380 mg, 88.1%) as a light yellow solid.

Step 2: To a solution of benzyl (2E)-3-(oxan-4-yl)prop-2-enoate (380 mg, 1.54 mmol, 1 eq) in THF (15 mL) was added Pd/C (100 mg, 0.94 mmol, 0.6 eq). The flask was evacuated and flushed three times with nitrogen, followed by flushing with hydrogen. The mixture was stirred at room temperature for 2 hours under an atmosphere of hydrogen (balloon). The resulting mixture was filtered, the filter cake was washed with MeOH (3×20 mL). The filtrate was concentrated under reduced pressure. This resulted in 3-(oxan-4-yl)propanoic acid (145 mg, 59.4%) as a white solid. MS: m/z: Calc'd for C₈H₁₄O₃[M+H]⁺159; Found, 159.

Step 3: To a stirred solution of 3-(oxan-4-yl)propanoic acid (60 mg, 0.38 mmol, 2 eq) in DCM (5 mL) were added DCC (59 mg, 0.28 mmol, 1.5 eq), DMAP (23.1 mg, 0.19 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq) at 0° C. under nitrogen atmosphere. The solution was stirred at room temperature for 12 h. The residue was purified by reversed-phase flash chromatography to afford tert-butyl (2R,3S,4S)-4-[(tert-butoxycarbonyl)oxy]-2-[(4-methoxyphenyl)methyl]-3-{[3-(oxan-4-yl)propanoyl]oxy}pyrrolidine-1-carboxylate (69 mg, 64.8%) as a light yellow solid. MS: m/z: Calc'd for: C₃₀H₄₅NO₉ [M+H−100−56]⁺408; Found, 408.

Step 4: To a stirred solution of tert-butyl (2R,3S,4S)-4-[(tert-butoxycarbonyl)oxy]-2-[(4-methoxyphenyl)methyl]-3-{[3-(oxan-4-yl)propanoyl]oxy}pyrrolidine-1-carboxylate (69 mg, 0.12 mmol, 1 eq) in DCM (5 mL) was added TFA (1 mL) dropwise at 0° C. under nitrogen atmosphere. The solution was stirred at room temperature for 2 h. The mixture was concentrated and the crude product (50 mg) was purified by Prep-HPLC to afford (2R,3S,4S)-4-hydroxy-2-[(4-methoxyphenyl)methyl]pyrrolidin-3-yl 3-(oxan-4-yl)propanoate; trifluoroacetic acid (18 mg, 30.7%) as a colorless solid. MS: m/z: Calc'd for C₂₀H₂₉NO₅ [M+H]⁺364; Found, 364. ¹H NMR (400 MHz, Methanol-d₄) δ 7.27-7.19 (m, 2H), 6.98-6.90 (m, 2H), 5.12 (d, J=3.4 Hz, 1H), 4.36 (d, J=4.4 Hz, 1H), 4.25-4.16 (m, 1H), 3.96 (dd, J=11.6, 4.4 Hz, 2H), 3.80 (s, 3H), 3.60 (dd, J=12.8, 4.4 Hz, 1H), 3.42 (t, J=11.6 Hz, 2H), 3.23 (d, J=12.7 Hz, 1H), 3.10 (dd, J=14.3, 6.8 Hz, 1H), 2.96 (dd, J=14.2, 8.7 Hz, 1H), 2.57-2.49 (m, 2H), 1.71-1.62 (m, 4H), 1.59-1.52 (m, 1H), 1.37-1.23 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 4% B to 34% B in 10 min; Wave Length: 254 nm/220 nm nm; RT1(min): 8.68

### Synthetic Procedure of V186992

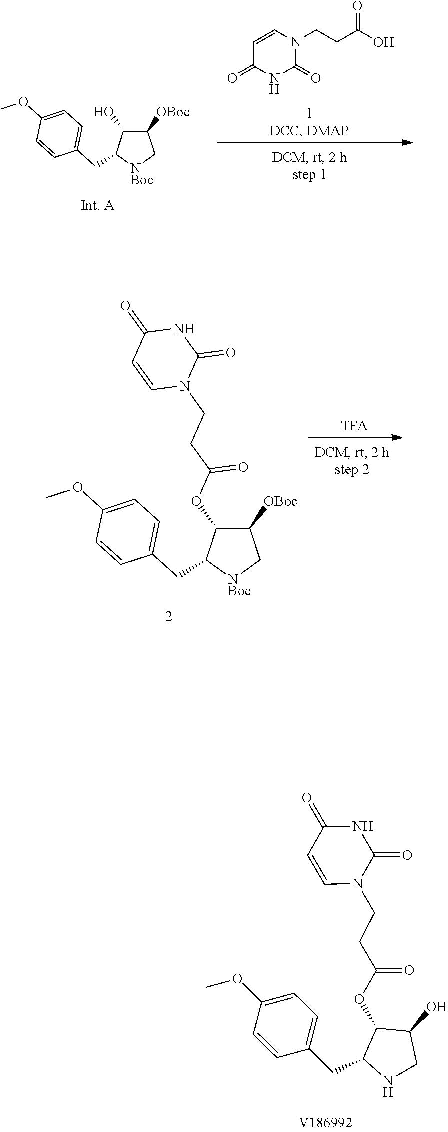

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate Int.A (100 mg, 0.01 mmol, 1 eq.) and 3-(2,4-dioxo-3H-pyrimidin-1-yl)propanoic acid (86.9 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, The reaction mixture was quenched with water and extracted with EA. The extracts were washed with brine, dried over anhydrous sodium sulfate, filtered and concentrated. The residue was purified by Prep-TLC (CH₂Cl₂/MeOH 10:1) to afford tert-butyl (2R,3S,4S)-4-[(tert-butoxy carbonyl)oxy]-3-{[3-(2,4-dioxo-3H-pyrimidin-1-yl)propanoyl]oxy}-2-[(4-methoxyphenyl) methyl]pyrrolidine-1-carboxylate (100 mg, 71.8%) as a white solid. MS: m/z: Calc'd for C₂₉H₃₉N₃O₁₀ [M−H]⁺588, found 588.

Step 2: To a stirred solution of tert-butyl (2R,3S,4S)-4-[(tert-butoxycarbonyl)oxy]-3-{[3-(2,4-dioxo-3H-pyrimidin-1-yl)propanoyl]oxy}-2-[(4-methoxyphenyl)methyl]pyrrolidine-1-carboxylate (95 mg, 0.16 mmol) in DCM (3 mL) was added TFA (1 mL) dropwise at 0° C. The reaction was stirred at r.t. for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(2,4-dioxo-3H-pyrimidin-1-yl)propanoate (24.7 mg) as a white solid. MS: m/z: Calc'd for C₁₉H₂₃N₃O₆ [M+H]⁺390, found 390. ¹H NMR (400 MHz, DMSO-d₆) δ 7.60-7.58 (m, 1H), 7.13-7.09 (m, 2H), 6.89-6.81 (m, 2H), 5.57 (dd, J=8.0, 2.6 Hz, 1H), 4.83-4.80 (m, 1H), 4.21 (d, J=4.3 Hz, 1H), 4.00 (dd, J=7.6, 3.3 Hz, 1H), 3.93-3.90 (m, 4H), 3.81 (s, 1H), 3.44 (dd, J=12.7, 4.5 Hz, 1H), 3.06 (d, J=12.7 Hz, 1H), 2.96-2.75 (m, 4H). Prep-HPLC-conditions: Column: Xselect CSH Prep C18 C18 Column, 30*150 mm, 5 μm; Mobile Phase A: Water(0.1% FA), Mobile Phase B: ACN; Flow rate: 60 mL/min mL/min; Gradient: 2% B to 23% B in 10 min; Wave Length: 254 nm/220 nm nm; RT1(min): 8.68

### Synthetic Procedure of V187006

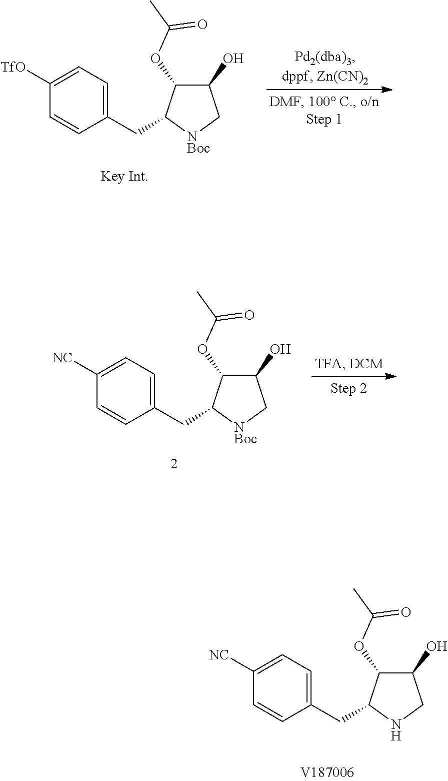 [SMILES: CC(=O)O[C@H]1[C@@H](Cc2ccc(OS(=O)(=O)C(F)(F)F)cc2)N(C(=O)OC(C)(C)C)C[C@@H]1O ; CC(=O)O[C@H]1[C@@H](Cc2ccc(C#N)cc2)N(C(=O)OC(C)(C)C)C[C@@H]1O ; CC(=O)O[C@@H]1[C@@H](O)CN[C@@H]1Cc1ccc(C#N)cc1]

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (240 mg, 0.49 mmol, 1 equiv) and zincdicarbonitrile (116.60 mg, 0.99 mmol, 2 equiv) in DMF (8 mL) was added Pd₂(dba)₃ (36.37 mg, 0.04 mmol, 0.08 equiv) and Dppf (43.87 mg, 0.07 mmol, 0.16 equiv) under N₂. The mixture was stirred at 100° C. for overnight under N₂. After completion of the reaction monitored by LCMS, the mixture was diluted with brine. The organic phase was dried anhydrous sodium sulfate, filtrated and concentrated. The residue was purified by a silica gel column to obtain (2R,3S,4S)-2-[(4-cyanophenyl)methyl]-4-hydroxypyrrolidin-3-yl acetate (130 mg, 90.6%) as a light yellow solid. MS: m/z: Calc'd for C₁₉H₂₄N₂O₅ [M+H]⁺361; found 361.

Step 2: To a stirred solution of tert-butyl (2R,3S,4S)-3-(acetyloxy)-4-hydroxy-2-{[4-(trifluoro methanesulfonyloxy)phenyl]methyl}pyrrolidine-1-carboxylate (60 mg, 0.124 mmol) in DCM (6 mL) was added trifluoroacetic acid (2 mL) at 0° C. The mixture was stirred at room temperature for 3 h. After completion of the reaction monitored by LCMS, the reaction mixture was concentrated. The residue was purified by Prep-HPLC to obtain (2R,3S,4S)-2-[(4-cyanophenyl) methyl]-4-hydroxypyrrolidin-3-yl acetate; trifluoroacetic acid (15.7 mg, 25.09%) as a white solid. MS: m/z: Calc'd for C₁₄H₁₆N₂O₃ [M+H]⁺261; found 261. ¹H NMR (400 MHz, Methanol-d₄) δ 7.79-7.73 (m, 2H), 7.57-7.51 (m, 2H), 5.11 (d, J=3.4 Hz, 1H), 4.39 (d, J=4.4 Hz, 1H), 4.29 (ddd, J=8.8, 6.6, 3.4 Hz, 1H), 3.64 (dd, J=12.7, 4.4 Hz, 1H), 3.26 (d, J=9.9, 4.5 Hz, 2H), 3.15 (dd, J=14.3, 9.0 Hz, 1H), 2.20 (s, 3H). Prep-HPLC purification condition: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 7% B to 37% B in 10 min; Wave Length: 254 nm/220 nm nm; RT1(min): 8.68

### Synthetic Procedure of V187008

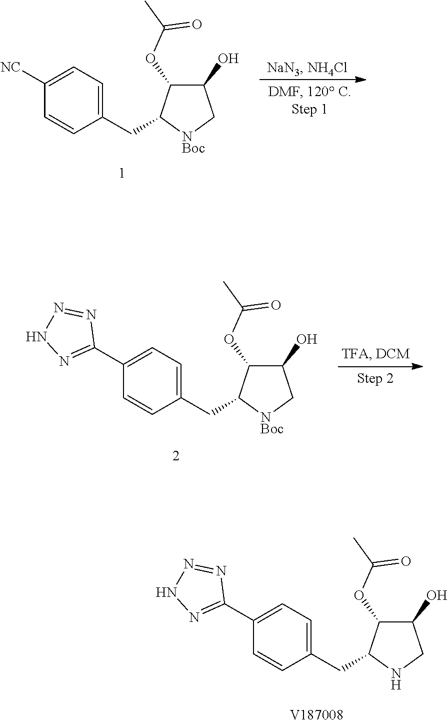 [SMILES: CC(=O)O[C@H]1[C@@H](Cc2ccc(C#N)cc2)N(C(=O)OC(C)(C)C)C[C@@H]1O ; CC(=O)O[C@H]1[C@@H](Cc2ccc(-c3nn[nH]n3)cc2)N(C(=O)OC(C)(C)C)C[C@@H]1O ; CC(=O)O[C@@H]1[C@@H](O)CN[C@@H]1Cc1ccc(-c2nn[nH]n2)cc1]

Step 1: To a stirred solution of tert-butyl (2R,3S,4S)-3-(acetyloxy)-2-[(4-cyanophenyl) methyl]-4-hydroxypyrrolidine-1-carboxylate (240 mg, 0.66 mmol, 1 equiv) and NH₄Cl (106.86 mg, 1.99 mmol, 3 equiv) in DMF (4 mL) was added a solution of NaN₃ (216.46 mg, 3.33 mmol, 5 equiv) in DMF (4 mL). The reaction was stirred at 120° C. for 24 h. LCMS showed the starting material was consumed completely. The reaction solution was injected into a reversed-phase column for purification. After the fraction was concentrated, tert-butyl (2R,3S,4S)-3-(acetyloxy)-4-hydroxy-2-{[4-(2H−1,2,3,4-tetrazol-5-yl)phenyl]methyl}pyrrolidine-1-carboxylate (130 mg, 48.3%) was obtained as a light-yellow solid. MS: m/z: Calc'd for C₁₉H₂₅N₅O₅ [M−H]⁻402; found 402.

Step 2: To a stirred solution of tert-butyl (2R,3S,4S)-3-(acetyloxy)-4-hydroxy-2-{[4-(2H-1,2,3,4-tetrazol-5-yl)phenyl]methyl}pyrrolidine-1-carboxylate (65 mg, 0.16 mmol, 1 equiv) in DCM (8 mL) was added TFA (4 mL). The mixture was stirred at room temperature for 3 h. After completion of the reaction monitored by LCMS, the mixture was concentrated. The residue was purified by Prep-HPLC to obtain (2R,3S,4S)-4-hydroxy-2-{[4-(2H−1,2,3,4-tetrazol-5-yl)phenyl]methyl}pyrrolidin-3-yl acetate; trifluoroacetic acid (12.8 mg, 18.92%) as an off-white solid. MS: m/z: Calc'd for C₁₄H₁₇N₅O₃ [M+H]⁺304; found 304. ¹H NMR (400 MHz, Methanol-d₄) δ 8.09-8.03 (m, 2H), 7.60-7.53 (m, 2H), 5.14 (d, J=3.4 Hz, 1H), 4.40 (d, J=4.3 Hz, 1H), 4.32 (dd, J=9.5, 3.4 Hz, 1H), 3.65 (dd, J=12.7, 4.4 Hz, 1H), 3.32-3.22 (m, 2H), 3.16 (dd, J=14.3, 8.9 Hz, 1H), 2.21 (s, 3H). Prep-HPLC purification condition: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 16% B in 10 min; Wave Length: 254 nm/220 nm nm; RT1(min): 8.68

### Synthetic Procedure of V187009

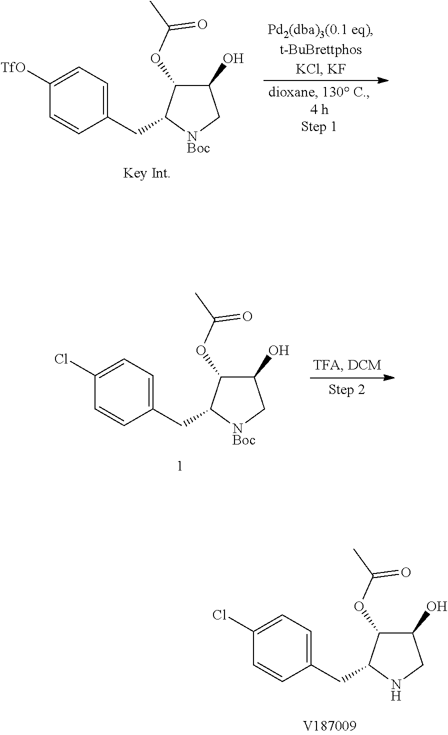 [SMILES: CC(=O)O[C@H]1[C@@H](Cc2ccc(OS(=O)(=O)C(F)(F)F)cc2)N(C(=O)OC(C)(C)C)C[C@@H]1O ; CC(=O)O[C@H]1[C@@H](Cc2ccc(Cl)cc2)N(C(=O)OC(C)(C)C)C[C@@H]1O ; CC(=O)O[C@@H]1[C@@H](O)CN[C@@H]1Cc1ccc(Cl)cc1]

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (170 mg, 0.35 mmol, 1 equiv), KCl (52.39 mg, 0.704 mmol, 2 equiv) and KF (10.21 mg, 0.176 mmol, 0.5 equiv) were added Pd₂(dba)₃ (25.76 mg, 0.02 mmol, 0.08 equiv) and di-tert-butyl[3,6-dimethoxy-2′,4′,6′-tris(propan-2-yl)-[1,1′-biphenyl]-2-yl]phosphane (40.90 mg, 0.08 mmol, 0.24 equiv) under N₂. The mixture was stirred at 130° C. for overnight under N₂. After completion of the reaction monitored by LCMS, the mixture was filtrated. The filtrate was concentrated, and the residue was purified by a reversed-phase column to obtain tert-butyl (2R,3S,4S)-3-(acetyloxy)-2-[(4-chlorophenyl)methyl]-4-hydroxypyrrolidine-1-carboxylate (80 mg, 61.5%) as a white solid. MS: m/z: Calc'd for C₁₈H₂₄ClNO₅ [M−56+H]+314; found 314.

Step 2: To a stirred solution of tert-butyl (2R,3S,4S)-3-(acetyloxy)-2-[(4-chlorophenyl) methyl]-4-hydroxypyrrolidine-1-carboxylate (75 mg, 0.20 mmol, 1 equiv) in DCM (8 mL) was added trifluoroacetic acid (4 mL) at 0° C. The mixture was stirred at room temperature for 3 h. After completion of the reaction monitored by LCMS, the reaction mixture was concentrated. The residue was purified by Prep-HPLC to obtain (2R,3S,4S)-2-[(4-chlorophenyl)methyl]-4-hydroxypyrrolidin-3-yl acetate; trifluoroacetic acid (25.3 mg, 32.38%) as a white solid. MS: m/z: Calc'd for C₁₃H₁₆ClNO₃ [M+H]⁺270; found 270. ¹H NMR (400 MHz, Methanol-d₄) δ 7.43-7.36 (m, 2H), 7.36-7.29 (m, 2H), 5.13-5.07 (m, 1H), 4.41-4.35 (m, 1H), 4.22 (ddd, J=9.0, 6.7, 3.4 Hz, 1H), 3.62 (dd, J=12.7, 4.4 Hz, 1H), 3.27-3.11 (m, 2H), 3.04 (dd, J=14.3, 8.9 Hz, 1H), 2.19 (s, 3H). Prep-HPLC purification condition: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm nm; RT1(min): 8.68

### Synthetic Procedure of V187010

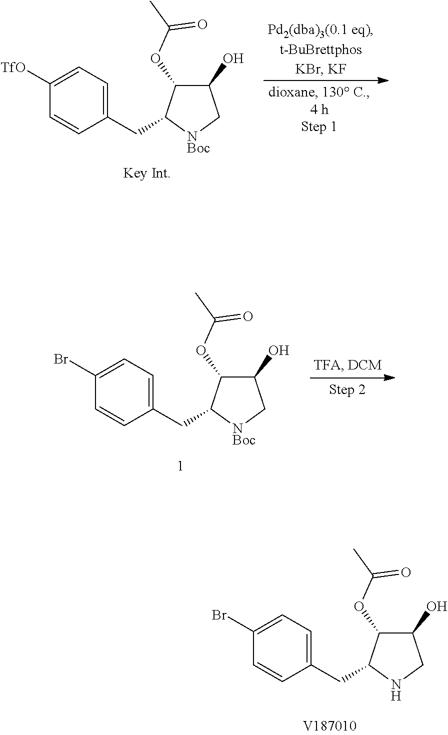 [SMILES: CC(=O)O[C@H]1[C@@H](Cc2ccc(OS(=O)(=O)C(F)(F)F)cc2)N(C(=O)OC(C)(C)C)C[C@@H]1O ; CC(=O)O[C@H]1[C@@H](Cc2ccc(Br)cc2)N(C(=O)OC(C)(C)C)C[C@@H]1O ; CC(=O)O[C@@H]1[C@@H](O)CN[C@@H]1Cc1ccc(Br)cc1]

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (300 mg, 0.62 mmol, 1 equiv), KBr (52.39 mg, 0.70 mmol, 2 equiv) and KF (18.03 mg, 0.31 mmol, 0.5 equiv) were added Pd₂(dba)₃ (28.41 mg, 0.031 mmol, 0.05 equiv) and di-tert-butyl[3,6-dimethoxy-2′,4′,6′-tris(propan-2-yl)-[1,1′-biphenyl]-2-yl]phosphane (45.11 mg, 0.09 mmol, 0.15 equiv) under N₂. The mixture was stirred at 130° C. for overnight under N₂. After completion of the reaction monitored by LCMS, the mixture was filtrated. The filtrate was concentrated, and the residue was purified by a reversed-phase column to obtain tert-butyl (2R,3S,4S)-3-(acetyloxy)-2-[(4-chlorophenyl)methyl]-4-hydroxypyrrolidine-1-carboxylate (180 mg, 78.4%) as a white solid. MS: m/z: Calc'd for C₁₈H₂₄BrNO₅ [M−56+H]+358; found 358.

Step 2: To a stirred solution of tert-butyl (2R,3S,4S)-3-(acetyloxy)-2-[(4-bromophenyl) methyl]-4-hydroxypyrrolidine-1-carboxylate (60 mg, 0.14 mmol, 1 equiv) in DCM (8 mL) was added trifluoroacetic acid (4 mL) at 0° C. The mixture was stirred at room temperature for 4 h. After completion of the reaction monitored by LCMS, the reaction mixture was concentrated. The residue was purified by Prep-HPLC to obtain (2R,3S,4S)-2-[(4-bromophenyl)methyl]-4-hydroxypyrrolidin-3-yl acetate; trifluoroacetic acid (25.7 mg, 40.5%) as a white solid. MS: m/z: Calc'd for C₁₃H₁₆BrNO₃ [M+H]⁺314; found 314. ¹HNMR (400 MHz, Methanol-d₄) δ 7.59-7.51 (m, 2H), 7.31-7.23 (m, 2H), 5.10 (d, J=3.4 Hz, 1H), 4.41-4.35 (m, 1H), 4.22 (ddd, J=9.4, 6.6, 3.4 Hz, 1H), 3.61 (dd, J=12.7, 4.4 Hz, 1H), 3.22 (d, J=12.7 Hz, 1H), 3.14 (dd, J=14.3, 6.6 Hz, 1H), 3.01 (dd, J=14.3, 8.9 Hz, 1H), 2.19 (s, 3H). Prep-HPLC purification condition: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm nm; RT1(min): 8.68

### Synthetic Procedure of V187035

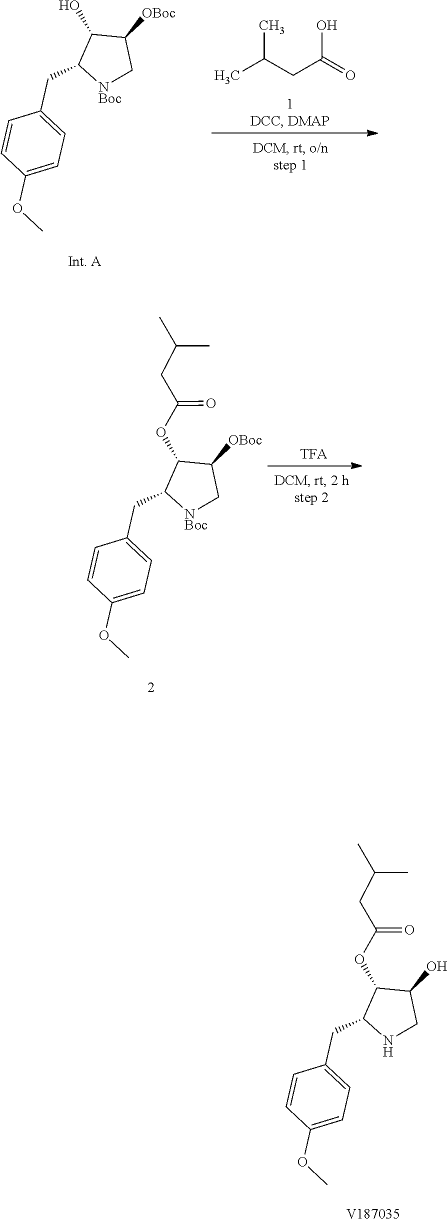

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and isovaleric acid (48.2 mg, 0.47 mmol, 2 eq.) in DCM (3 mL) was added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, the resulting mixture was filtered, the filter cake was washed with DCM (1×10 mL). The filtrate was concentrated under reduced pressure. The residue was purified by Prep-TLC (CH₂Cl₂/MeOH 10:1) to afford tert-butyl (2R,3S,4S)-4-[(tert-butoxycarbonyl)oxy]-2-[(4-methoxyphenyl)methyl]-3-[(3-methyl butanoyl)oxy]pyrrolidine-1-carboxylate (90 mg, 75.0%) as a white solid. MS: m/z: Calc'd for C₂₇H₄₁NO₈ [M+H−56−56]⁺396, found 396.

Step 2: To a stirred solution of tert-butyl (2R,3S,4S)-4-[(tert-butoxycarbonyl)oxy]-2-[(4-methoxyphenyl)methyl]-3-[(3-methylbutanoyl)oxy]pyrrolidine-1-carboxylate (85 mg, 0.16 mmol, 1 eq.) in DCM (3 mL) was added TFA (1 mL) dropwise at 0° C. The reaction was stirred at r.t. for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-methylbutanoate (27.4 mg, 52.7%) as a white solid. MS: m/z: Calc'd for C₁₇H₂₅NO₄ [M+H]⁺308, found 308. ¹H NMR (400 MHz, Methanol-d₄) δ 7.27-7.20 (m, 2H), 6.94-6.91 (m, 2H), 5.12 (d, J=3.4 Hz, 1H), 4.35 (d, J=4.4 Hz, 1H), 4.25-4.15 (m, 1H), 3.80 (s, 3H), 3.58-3.54 (m, 1H), 3.30 (s, 1H), 3.22 (d, J=12.7 Hz, 1H), 3.10-3.06 (m, 1H), 2.38 (d, J=7.1 Hz, 2H), 2.16-2.12 (m, 1H), 1.03-0.98 (m, 6H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 8% B to 38% B in 10 min; Wave Length: 254 nm/220 nm nm; RT1(min): 8.68

### Synthetic Procedure of V187041

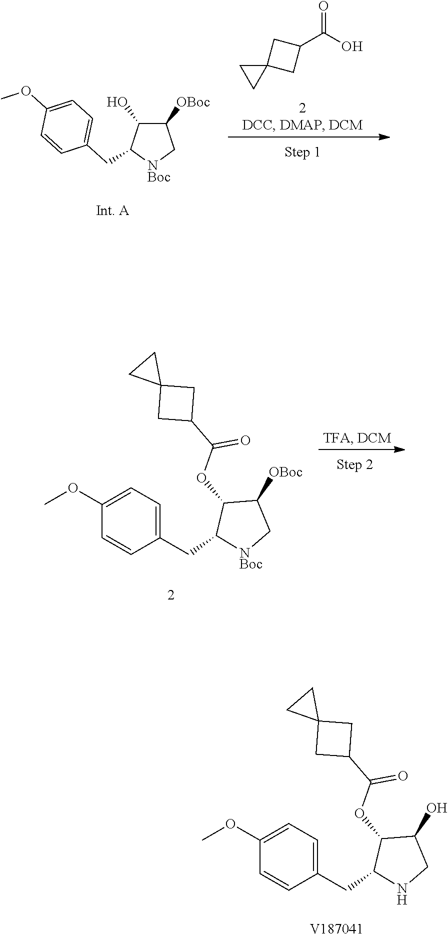

Step 1: A solution of spiro[2.3]hexane-5-carboxylic acid (47.6 mg, 0.37 mmol, 2 equiv) in DCM (4 mL) was treated with DCC (58.4 mg, 0.28 mmol, 1.5 equiv) and DMAP (23.08 mg, 0.189 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl) methyl]pyrrolidine-1-carboxylate (80 mg, 0.18 mmol, 1 equiv) at 0° C. The resulting mixture was stirred for overnight at room temperature. Desired product could be detected by LCMS. The mixture was concentrated to give the crude product which was purified by Prep-TLC (PE/EA 3:1) to afford tert-butyl (2R,3S,4S)-4-[(tert-butoxycarbonyl)oxy]-2-[(4-methoxyphenyl) methyl]-3-{spiro[2.3]hexane-5-carbonyloxy}pyrrolidine-1-carboxylate (56 mg, 55.7%) as a white oil. MS: m/z: Calc'd for C₂₉H₄₁NO₈[M+H−56−56]⁺420, found 420.

Step 2: To a stirred solution of tert-butyl (2R,3S,4S)-4-[(tert-butoxycarbonyl)oxy]-2-[(4-methoxyphenyl)methyl]-3-{spiro[2.3]hexane-5-carbonyloxy}pyrrolidine-1-carboxylate (56 mg, 0.10 mmol, 1 equiv) in DCM (2.5 mL) was added TFA (0.5 mL) at 0° C. The resulting mixture was stirred for 1 h at room temperature under air atmosphere. Desired product could be detected by LCMS. The mixture was concentrated to give the crude product (45 mg) which was purified by Prep-HPLC to afford (2R,3S,4S)-4-hydroxy-2-[(4-methoxyphenyl)methyl]pyrrolidin-3-yl spiro[2.3]hexane-5-carboxylate; trifluoroacetic acid (29.8 mg, 63.3%) as a white solid.

MS: m/z: Calc'd for C₂₅H₃₄FNO₈[M+H]⁺332, found 332. ¹H NMR (400 MHz, Methanol-d₄) δ 7.26-7.18 (m, 2H), 6.93 (d, J=8.3 Hz, 2H), 5.11 (d, J=3.5 Hz, 1H), 4.36 (d, J=4.3 Hz, 1H), 4.21-4.11 (m, 1H), 3.79 (s, 3H), 3.58 (dd, J=13.0, 4.2 Hz, 1H), 3.47-3.35 (m, 1H), 3.23 (d, J=12.7 Hz, 1H), 3.09 (dd, J=14.2, 7.0 Hz, 1H), 3.02-2.92 (m, 1H), 2.54-2.41 (m, 2H), 2.46-2.29 (m, 2H), 0.60-0.50 (m, 2H), 0.50-0.43 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 12% B to 42% B in 10 min; Wave Length: 254 nm/220 nm nm; RT1(min): 8.68

### Synthetic Procedure of V187042

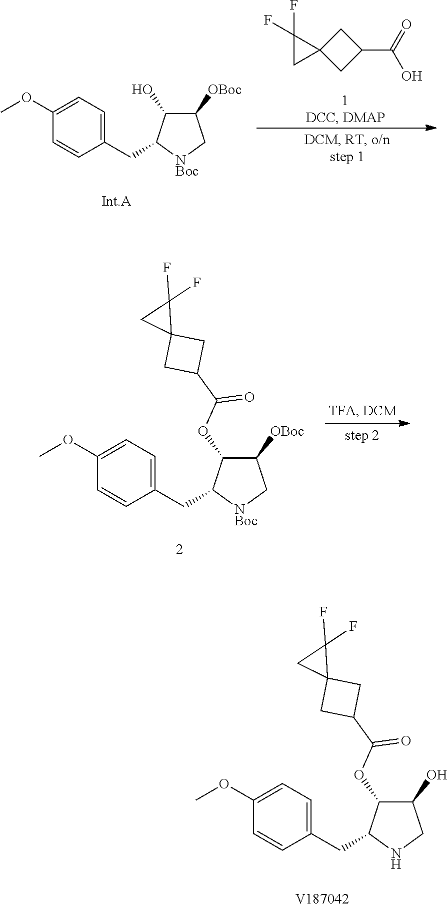

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1 eq.) and 1,1-difluorospiro[2.3]hexane-5-carboxylic acid (61.3 mg, 0.38 mmol, 2 eq.) in DCM (5 mL) were added DCC (58.5 mg, 0.28 mmol, 1.5 eq.) and DMAP (23.1 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The residue was purified by Prep-TLC (PE/EA 5:1 as eluent) to afford tert-butyl (2R,3S,4S)-4-[(tert-butoxy carbonyl)oxy]-3-{1,1-difluorospiro[2.3]hexane-5-carbonyloxy}-2-[(4-methoxyphenyl)methyl]pyrrolidine-1-carboxylate (60 mg, 55.9%) as a light yellow oil. MS: m/z: Calc'd for C₂₉H₃₉F₂NO₈[M+H−56−100]⁺412, found 412.

Step 2: To a stirred mixture of tert-butyl (2R,3S,4S)-4-[(tert-butoxycarbonyl)oxy]-3-{1,1-difluorospiro[2.3]hexane-5-carbonyloxy}-2-[(4-methoxyphenyl)methyl]pyrrolidine-1-carboxylate (55 mg, 0.10 mmol) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 1,1-difluorospiro[2.3]hexane-5-carboxylate; trifluoroacetic acid (18.4 mg, 38.9%) as a white solid. MS: m/z: Calc'd for C₁₉H₂₃F₂NO₄ [M+H]⁺368, found 368. ¹H NMR (400 MHz, Methanol-d4) δ 7.23 (d, J=8.0 Hz, 2H), 6.94 (d, J=8.1 Hz, 2H), 5.14 (d, J=3.4 Hz, 1H), 4.39 (d, J=4.3 Hz, 1H), 4.25-4.20 (m, 1H), 3.80 (s, 3H), 3.58 (dd, J=12.6, 4.2 Hz, 1H), 3.48-3.40 (m, 1H), 3.23 (d, J=12.6 Hz, 1H), 3.10 (dd, J=14.3, 6.7 Hz, 1H), 2.95 (dd, J=14.6, 9.1 Hz, 1H), 2.67-2.47 (m, 4H), 1.34-1.30 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 10% B to 40% B in 10 min; Wave Length: 254 nm/220 nm nm; RT1(min): 8.68

### Synthetic Procedure of V187043

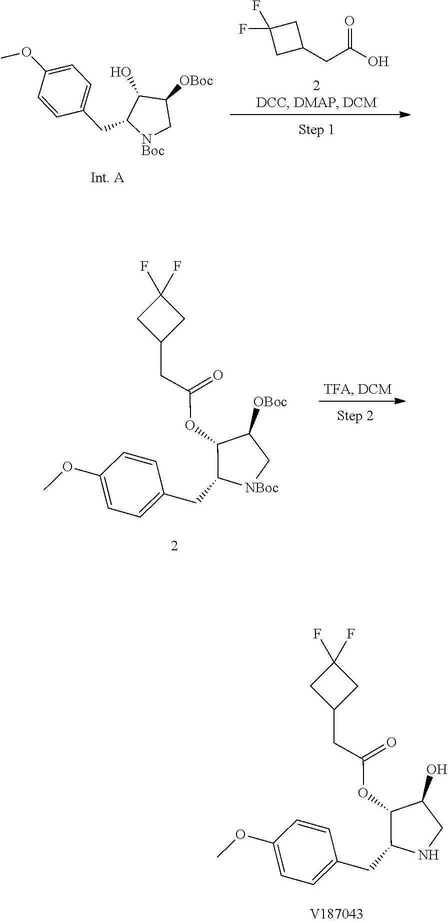

Step 1: A solution of (3,3-difluorocyclobutyl)acetic acid (56.7 mg, 0.37 mmol, 2 equiv) in DCM (1 mL) was treated with DCC (58.4 mg, 0.28 mmol, 1.5 equiv) and DMAP (23.0 mg, 0.18 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.18 mmol, 1 equiv). The resulting mixture was stirred for overnight at room temperature under nitrogen atmosphere. Desired product could be detected by LCMS. The mixture was concentrated and the residue was purified by Prep-TLC (PE/EA 3:1) to afford tert-butyl (2R,3S,4S)-4-[(tert-butoxycarbonyl)oxy]-3-{[2-(3,3-difluorocyclobutyl)acetyl]oxy}-2-[(4-methoxyphenyl)methyl]pyrrolidine-1-carboxylate (75 mg, 71.4%) as a colorless oil. MS: m/z: Calc'd for C₂₈H₃₉F₂NO₈[M+H−56−56]⁺444, found 444.

Step 2: To a stirred solution of tert-butyl (2R,3S,4S)-4-[(tert-butoxycarbonyl)oxy]-3-{[2-(3,3-difluorocyclobutyl)acetyl]oxy}-2-[(4-methoxyphenyl)methyl]pyrrolidine-1-carboxylate (80 mg, 0.14 mmol, 1 equiv) in DCM (2.5 mL) was added TFA (0.5 mL) at 0° C. under air atmosphere. The resulting mixture was stirred for 1 h at room temperature. Desired product could be detected by LCMS. The mixture was concentrated to give the crude (50 mg), which was purified by Prep-HPLC to afford (2R,3S,4S)-4-hydroxy-2-[(4-methoxyphenyl)methyl]pyrrolidin-3-yl 2-(3,3-difluorocyclobutyl)acetate; trifluoroacetic acid (25.8 mg, 38.1%) as a white solid. MS: m/z: Calc'd for C₁₈H₂₃F₂NO₄ [M+H]⁺356, found 356. ¹H NMR (400 MHz, Methanol-d₄) δ 7.27-7.19 (m, 2H), 6.97-6.89 (m, 2H), 5.10 (d, J=3.4 Hz, 1H), 4.38-4.32 (m, 1H), 4.20-4.05 (m, 1H), 3.79 (s, 3H), 3.60 (dd, J=12.7, 4.4 Hz, 1H), 3.22 (d, J=12.7 Hz, 1H), 3.08 (dd, J=14.3, 6.9 Hz, 1H), 2.97 (dd, J=14.3, 8.7 Hz, 1H), 2.87-2.66 (m, 4H), 2.60-2.49 (m, 1H), 2.43-2.24 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 9% B to 39% B in 10 min; Wave Length: 254 nm/220 nm nm; RT1(min): 8.68

### Synthetic Procedure of V187044

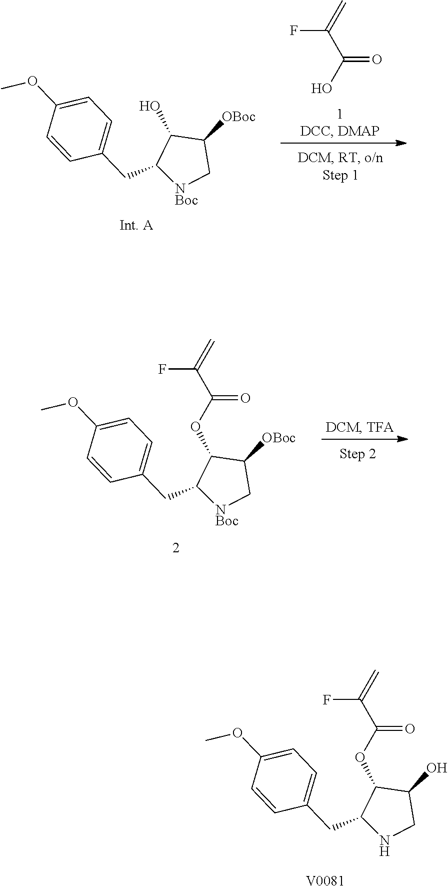

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and 3-fluorobicyclo[1.1.1]pentane-1-carboxylic acid (49.2 mg, 0.38 mmol, 2 eq.) in DCM (5 mL) were added DCC (58.46 mg, 0.28 mmol, 1.5 eq.) and DMAP (23.1 mg, 0.19 mmol, 1 eq.) at 0° C. under nitrogen atmosphere. The resulting reaction mixture was stirred at ambient temperature for overnight. Upon completion, the reaction mixture was concentrated in vacuo. The residue was purified by Prep-TLC (PE/EA 5:1) to afford tert-butyl (2R,3S,4S)-4-[(tert-butoxycarbonyl) oxy]-3-{3-fluorobicyclo[1.1.1]pentane-1-carbonyloxy}-2-[(4-methoxyphenyl)methyl]pyrrolidine-1-carboxylate (70 mg, 69.19%) as a light yellow oil. MS: m/z: Calc'd for C₂₈H₃₈FNO₈[M+H−56−100]⁺380, found 380.

Step 2: To a stirred mixture of tert-butyl (2R,3S,4S)-4-[(tert-butoxycarbonyl)oxy]-3-{3-fluorobicyclo[1.1.1]pentane-1-carbonyloxy}-2-[(4-methoxyphenyl)methyl]pyrrolidine-1-carboxylate (65 mg, 0.12 mmol) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-fluorobicyclo[1.1.1]pentane-1-carboxylate; trifluoroacetic acid (13.6 mg, 24.7%) as a light yellow solid. MS: m/z: Calc'd for C₁₅H₁₈FNO₄ [M+H]⁺336, found 336. ¹H NMR (400 MHz, Methanol-d₄) δ 7.25-7.16 (m, 2H), 6.93 (d, J=8.1 Hz, 2H), 5.11-5.05 (m, 1H), 4.36 (d, J=4.2 Hz, 1H), 4.26-4.21 (m, 1H), 3.80 (s, 3H), 3.57 (dd, J=12.9, 4.1 Hz, 1H), 3.25 (d, J=12.7 Hz, 1H), 3.06 (dd, J=14.2, 7.2 Hz, 1H), 3.01-2.91 (m, 1H), 2.47 (d, J=2.5 Hz, 6H). Prep-HPLC-conditions: Column: Xselect CSH Prep C18 Column, 30*150 mm, 5 μm; Mobile Phase A: Water(0.05% TFA), Mobile Phase B: ACN; Flow rate: 60 mL/min mL/min; Gradient: 7% B to 37% B in 10 min; Wave Length: 254 nm/220 nm nm; RT1(min): 8.68

### Synthetic Procedure of V187045

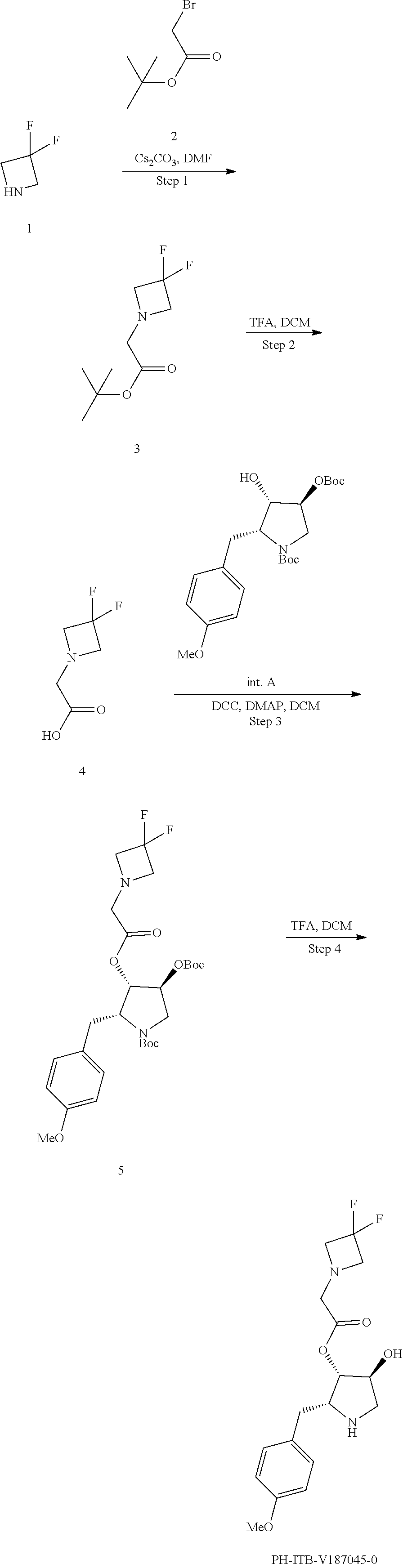

Step 1: To a stirred solution of tert-butyl 2-bromoacetate (226.8 mg, 1.16 mmol, 1.5 equiv) and 3,3-difluoroazetidine (100 mg, 0.77 mmol, 1 equiv) in DMF (10 mL) was added Cs₂CO₃ (757.7 mg, 2.32 mmol, 3 equiv) at room temperature. The mixture was stirred for overnight. Upon completion, the resulting mixture was quenched with water, extracted with EA (3*50 mL). The combined organic layers were dried over anhydrous Na₂SO₄. After filtration, the filtrate was concentrated under reduced pressure to get crude product (150 mg), which was used in the next step directly without further purification. MS: m/z: Calc'd for C₉H₁₅F₂NO₂ [M+H]⁺208, found 208.

Step 2: To a solution of tert-butyl 2-(3,3-difluoroazetidin-1-yl)acetate (100 mg, 0.48 mmol) in DCM (9 mL) was added TFA (3 mL). The mixture was stirred for 2 hours at room temperature. Upon completion, the reaction mixture was concentrated in vacuo to get crude product (120 mg). The crude product was used in the next step directly without further purification. MS: m/z: Calc'd for C₅H₇F₂NO₂ [M+H]⁺152, found 152.

Step 3: A solution of (3,3-difluoroazetidin-1-yl)acetic acid (120 mg, 0.794 mmol, 2 equiv) in DCM (10 mL) was treated with tert-butyl (2R,3S,4S)-4-[(tert-butoxycarbonyl)oxy]-3-hydroxy-2-[(4-methoxyphenyl)methyl]pyrrolidine-1-carboxylate (168.15 mg, 0.397 mmol, 1.00 equiv), DCC (245.78 mg, 1.191 mmol, 3 equiv) and DMAP (72.76 mg, 0.596 mmol, 1.5 equiv) at 0° C. The mixture was stirred overnight at room temperature. Upon completion, the reaction mixture was concentrated in vacuo. The residue was purified by Prep-TLC (PE/EA, 2/1) to afford tert-butyl (2R,3S,4S)-4-[(tert-butoxycarbonyl)oxy]-3-{[2-(3,3-difluoroazetidin-1-yl)acetyl]oxy}-2-[(4-methoxyphenyl)methyl]pyrrolidine-1-carboxylate) (100 mg 45.2%) as a yellow oil. MS: m/z: Calc'd for C₂₇H₃₈F₂N₂O₈ [M+H]⁺557, found 557.

Step 4: To a solution of tert-butyl (2R,3S,4S)-4-[(tert-butoxycarbonyl)oxy]-3-{[2-(3,3-difluoroazetidin-1-yl)acetyl]oxy}-2-[(4-methoxyphenyl)methyl]pyrrolidine-1-carboxylate (100 mg, 0.180 mmol, 1 equiv) in DCM (9 mL) was added TFA (3 mL). The mixture was stirred for 2 hours at room temperature. Upon completion, the reaction mixture was concentrated in vacuo. The residue was purified by Prep-HPLC to afford (2R,3S,4S)-4-hydroxy-2-[(4-methoxyphenyl) methyl]pyrrolidin-3-yl 2-(3,3-difluoroazetidin-1-yl)acetate; trifluoroacetic acid) as a white solid. MS: m/z: Calc'd for C₁₇H₂₂F₂N₂O₄[M+H]⁺357, found 357. ¹H NMR (400 MHz, Methanol-d₄) δ 7.28-7.18 (m, 2H), 6.94 (m, 2H), 5.24-5.09 (m, 1H), 4.40 (d, J=4.4 Hz, 1H), 4.23-4.21 (m, 1H), 4.11 (s, 3H), 3.87-3.85 (m, 2H), 3.80-3.78 (m, 4H), 3.60 (d, J=4.7 Hz, 1H), 3.23-3.21 (m, 1H), 3.09 (m, 1H), 2.97 (m, 1H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm nm; RT1(min): 8.68

### Synthetic Procedure of V187048

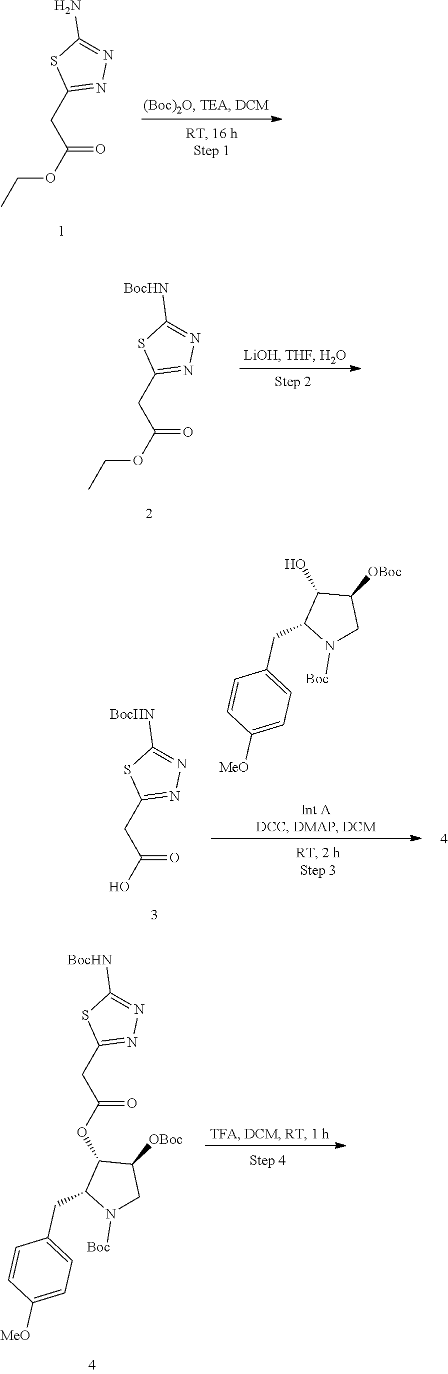

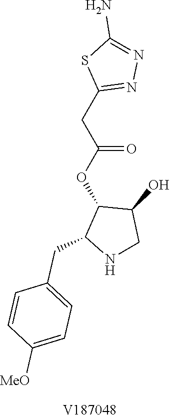 [SMILES: COc1ccc(C[C@H]2NC[C@H](O)[C@H]2OC(=O)Cc2nnc(N)s2)cc1]

Step 1: To a stirred solution of ethyl 2-(5-amino-1,3,4-thiadiazol-2-yl)acetate (100 mg, 0.53 mmol, 1 equiv) and (Boc)₂O (233 mg, 1.06 mmol, 2 equiv) in DCM (4 mL) was added TEA (162 mg, 1.60 mmol, 3 equiv) at 0° C. The resulting mixture was stirred for overnight at room temperature. Desired product could be detected by LCMS. The mixture was concentrated. The residue was purified by Prep-TLC (PE/EA=1:2) to afford ethyl 2-{5-[(tert-butoxycarbonyl) amino]-1,3,4-thiadiazol-2-yl}acetate (60 mg, 39.1%) as a white solid.

MS: m/z: Calc'd for C₁₁H₁₇N₃O₄S [M+H]⁺288, found 288.

Step 2: To a stirred solution of ethyl 2-{5-[(tert-butoxycarbonyl)amino]-1,3,4-thiadiazol-2-yl}acetate (150 mg, 0.52 mmol, 1 equiv) in THF (3 mL) and H₂O (1.5 mL) was added LiOH (125 mg, 5.22 mmol, 10 equiv) at room temperature under air atmosphere. The resulting mixture was stirred for overnight at room temperature. Desired product could be detected by LCMS. The resulting mixture was concentrated under reduced pressure. The crude product (100 mg) was purified by reversed-phase column chromatography to afford {5-[(tert-butoxycarbonyl)amino]-1,3,4-thiadiazol-2-yl}acetic acid (63 mg, 46.5%) as a white solid. MS: m/z: Calc'd for C₉H₁₃N₃O₄S [M+H−56]⁺204, found 204.

Step 3: A solution of {5-[(tert-butoxycarbonyl)amino]-1,3,4-thiadiazol-2-yl}acetic acid (55 mg, 0.21 mmol, 1.00 equiv) in DCM (5 mL) was treated with DCC (66 mg, 0.31 mmol, 1.5 equiv) and DMAP (26 mg, 0.21 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxy phenyl)methyl]pyrrolidine-1-carboxylate (108 mg, 0.25 mmol, 1.2 equiv). The resulting mixture was stirred for overnight at room temperature. Desired product could be detected by LCMS. The resulting mixture was filtered, the filter cake was washed with CH₂Cl₂ (2×20 mL). The filtrate was concentrated under reduced pressure. The residue was purified by Prep-TLC (PE/EA=1:1) to afford tert-butyl (2R,3S,4S)-3-[(2-{5-[(tert-butoxycarbonyl)amino]-1,3,4-thiadiazol-2-yl}acetyl)oxy]-4-[(tert-butoxycarbonyl)oxy]-2-[(4-methoxyphenyl)methyl]pyrrolidine-1-carboxylate (75 mg, 53.1%) as a white solid. MS: m/z: Calc'd for C₃₁H₄₄N₄O₁₀S [M+H]⁺665, found 665.

Step 4: To a stirred solution of tert-butyl (2R,3S,4S)-3-[(2-{5-[(tert-butoxycarbonyl)amino]-1,3,4-thiadiazol-2-yl}acetyl)oxy]-4-[(tert-butoxycarbonyl)oxy]-2-[(4-methoxyphenyl)methyl]pyrrolidine-1-carboxylate (75 mg, 0.11 mmol, 1 equiv) in DCM (5 mL) was added TFA (1 mL) at 0° C. The resulting mixture was stirred for 1 h at room temperature. Desired product could be detected by LCMS. The mixture was concentrated under vacuum. The crude product (60 mg) was purified by Prep-HPLC to afford (2R,3S,4S)-4-hydroxy-2-[(4-methoxyphenyl)methyl]pyrrolidin-3-yl 2-(5-amino-1,3,4-thiadiazol-2-yl)acetate (20.8 mg, 50.4%) as a white solid.

MS: m/z: Calc'd for C₁₆H₂₀N₄O₄S [M+H]⁺365, found 365. 1H NMR (400 MHz, Methanol-d₄) δ 7.26-7.18 (m, 2H), 6.96-6.88 (m, 2H), 5.14 (d, J=3.4 Hz, 1H), 4.46-4.40 (m, 1H), 4.24-4.11 (m, 1H), 3.79 (s, 3H), 3.62-3.49 (m, 1H), 3.34-3.32 (m, 2H), 3.25 (d, J=12.7 Hz, 1H), 3.09 (dd, J=14.3, 7.0 Hz, 1H), 3.04-2.94 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 18% B in 10 min; Wave Length: 254 nm/220 nm; RT1(min): 8.68

### Synthetic Procedure of V187049

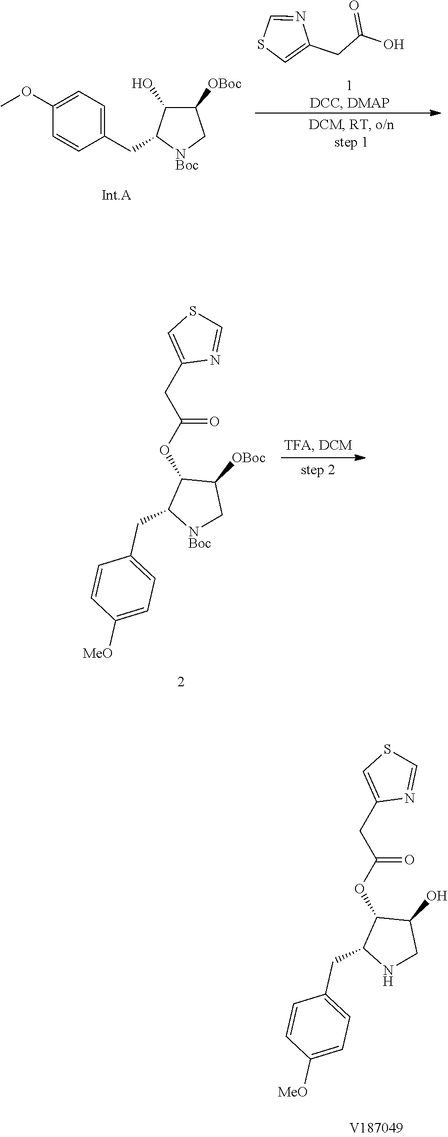

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and 1,3-thiazol-4-ylacetic acid (54.9 mg, 0.38 mmol, 2.0 eq.) in DCM (5 mL) were added DCC (58.5 mg, 0.28 mmol, 1.5 eq.) and DMAP (23.1 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-{[2-(1,3-thiazol-4-yl)acetyl]oxy}pyrrolidine-1-carboxylate (60 mg, 57.9%) as a light yellow oil. MS: m/z: Calc'd for C₂₇H₃₆N₂O₈S [M+H]⁺549, found 549.

Step 2: To a stirred mixture of tert-butyl (2R,3S,4S)-4-[(tert-butoxycarbonyl)oxy]-2-[(4-methoxyphenyl)methyl]-3-{[2-(1,3-thiazol-4-yl)acetyl]oxy}pyrrolidine-1-carboxylate (55 mg, 0.10 mmol, 1.0 eq.) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford 2R,3S,4S)-4-hydroxy-2-[(4-methoxyphenyl)methyl]pyrrolidin-3-yl 2-(1,3-thiazol-4-yl)acetate; trifluoroacetic acid (18.5 mg, 39.9%) as a white solid. MS: m/z: Calc'd for C₁₇H₂₀N₂O₄S [M+H]⁺349, found 349. ¹H NMR (400 MHz, Methanol-d₄) δ 9.08 (d, J=2.0 Hz, 1H), 7.59 (d, J=2.0 Hz, 1H), 7.19-7.11 (m, 2H), 6.95-6.87 (m, 2H), 5.11-5.06 (m, 1H), 4.42-4.36 (m, 1H), 4.20-4.16 (m, 1H), 4.13-3.99 (m, 2H), 3.79 (s, 3H), 3.60 (dd, J=12.8, 4.4 Hz, 1H), 3.23 (d, J=12.7 Hz, 1H), 3.02-3.00 (m, 1H), 2.94-2.88 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.68

### Synthetic Procedure of V187050

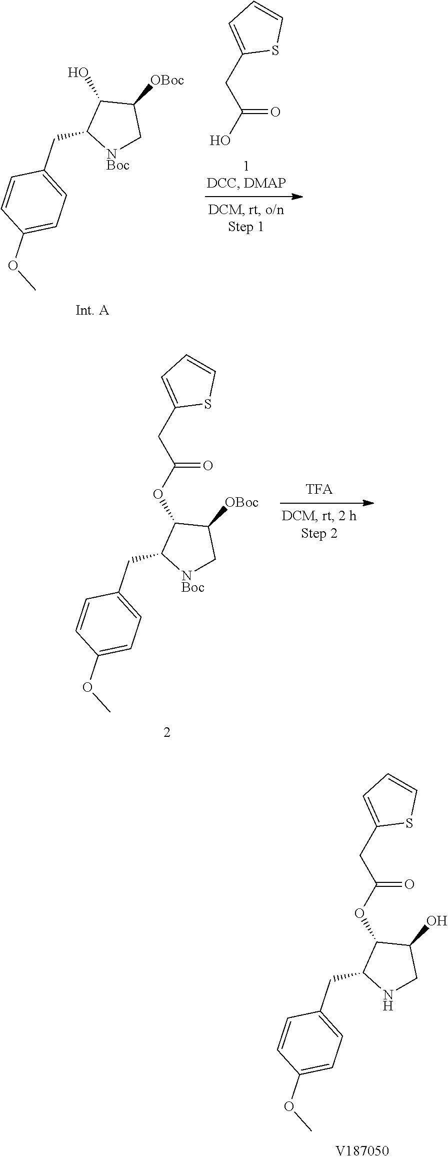

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.236 mmol, 1 eq.) and thiolacetic acid (67.1 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, the resulting mixture was filtered, the filter cake was washed with DCM (1×10 mL). The filtrate was concentrated under reduced pressure. The residue was purified by Prep-TLC (CH₂Cl₂/MeOH 10:1) to afford tert-butyl (2R,3S,4S)-4-[(tert-butoxycarbonyl)oxy]-2-[(4-methoxyphenyl)methyl]-3-{[2-(thiophen-2-yl)acetyl]oxy}pyrrolidine-1-carboxylate (90 mg, 69.6%) as a white solid.

MS: m/z: Calc'd for C₂₈H₃₇NO₈S [M−H−56−56]⁺435, found 435.

Step 2: To a stirred solution of tert-butyl (2R,3S,4S)-4-[(tert-butoxycarbonyl)oxy]-2-[(4-methoxyphenyl)methyl]-3-{[2-(thiophen-2-yl)acetyl]oxy}pyrrolidine-1-carboxylate (85 mg, 0.15 mmol, 1 eq.) in DCM (3 mL) was added TFA (1 mL) dropwise at 0° C. The reaction was stirred at r.t. for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(thiophen-2-yl)acetate (26.2 mg, 48.4%) as a white solid. MS: m/z: Calc'd for C₁₈H₂₁NO₄S [M+H]⁺348, found 348. ¹H NMR (400 MHz, Methanol-d₄) δ 7.38-7.35 (m, 1H), 7.14-7.05 (m, 3H), 7.04-7.01 (m, 1H), 6.92-6.84 (m, 2H), 5.06 (d, J=3.5 Hz, 1H), 4.35 (d, J=4.3 Hz, 1H), 4.20-4.15 (m, 1H), 4.11-3.97 (m, 2H), 3.78 (s, 3H), 3.57-3.54 (m, 1H), 3.24 (d, J=12.7 Hz, 1H), 3.03-3.00 (m, 1H), 2.93-2.86 (m, 1H). Prep-HPLC conditions: Column: XSelect CSH Prep C18 OBD Column, 5 μm, 19*150 mm; Mobile Phase A:Water (0.05% TFA), Mobile Phase B:ACN; Flow rate:25 mL/min; Gradient:38 B to 50 B in 10 min; 254/210 nm; RT1:10.48; RT2:; Injection Volumn: ml; Number Of Runs:

### Synthetic Procedure of V187052

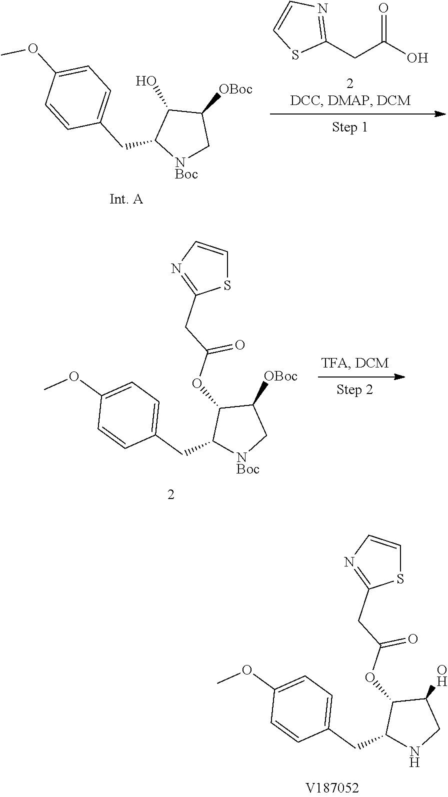

Step 1: A solution of 3-cyclopropylpropionic acid (29.1 mg, 0.25 mmol, 2 equiv) in DCM (1 mL) was treated with DCC (39.4 mg, 0.19 mmol, 1.5 equiv) and DMAP (15.5 mg, 0.12 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (54 mg, 0.12 mmol, 1 equiv). The resulting mixture was stirred for overnight at room temperature under nitrogen atmosphere. Desired product could be detected by LCMS. The mixture was concentrated. The residue was purified by Prep-TLC (PE/EA=5:1) to afford tert-butyl (2R,3S,4S)-4-[(tert-butoxycarbonyl)oxy]-3-[(3-cyclopropylpropanoyl)oxy]-2-[(4-methoxyphenyl)methyl]pyrrolidine-1-carboxylate (50 mg, 57.3%) as a yellow oil.

MS: m/z: Calc'd for C₂₇H₃₆N₂O₈S [M+H]⁺549, found 549.

Step 2: To a stirred solution of tert-butyl (2R,3S,4S)-4-[(tert-butoxycarbonyl)oxy]-2-[(4-methoxyphenyl)methyl]-3-{[2-(1,3-thiazol-2-yl)acetyl]oxy}pyrrolidine-1-carboxylate (50 mg, 0.09 mmol, 1 equiv) in DCM (2.5 mL) was added TFA (0.5 mL) at 0° C. The resulting mixture was stirred for 1 h at room temperature under air atmosphere. Desired product could be detected by LCMS. The resulting mixture was concentrated under reduced pressure. The crude product (40 mg) was purified by Prep-HPLC to afford (2R,3S,4S)-4-hydroxy-2-[(4-methoxyphenyl) methyl]pyrrolidin-3-yl 2-(1,3-thiazol-2-yl)acetate; trifluoroacetic acid (26.5 mg, 62.8%) as a white solid. MS: m/z: Calc'd for C₂₅H₃₄FNO₈[M+H]⁺349, found 349. ¹H NMR (400 MHz, Methanol-d₄) δ 7.84 (d, J=3.4 Hz, 1H), 7.65 (d, J=3.4 Hz, 1H), 7.20-7.12 (m, 2H), 6.94-6.86 (m, 2H), 5.10 (d, J=3.3 Hz, 1H), 4.44-4.38 (m, 1H), 4.21-4.12 (m, 1H), 3.78 (s, 3H), 3.61 (dd, J=12.7, 4.5 Hz, 1H), 3.24 (d, J=12.7 Hz, 1H), 3.09-2.88 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm nm; RT1(min): 8.68

### Synthetic Procedure of V187053

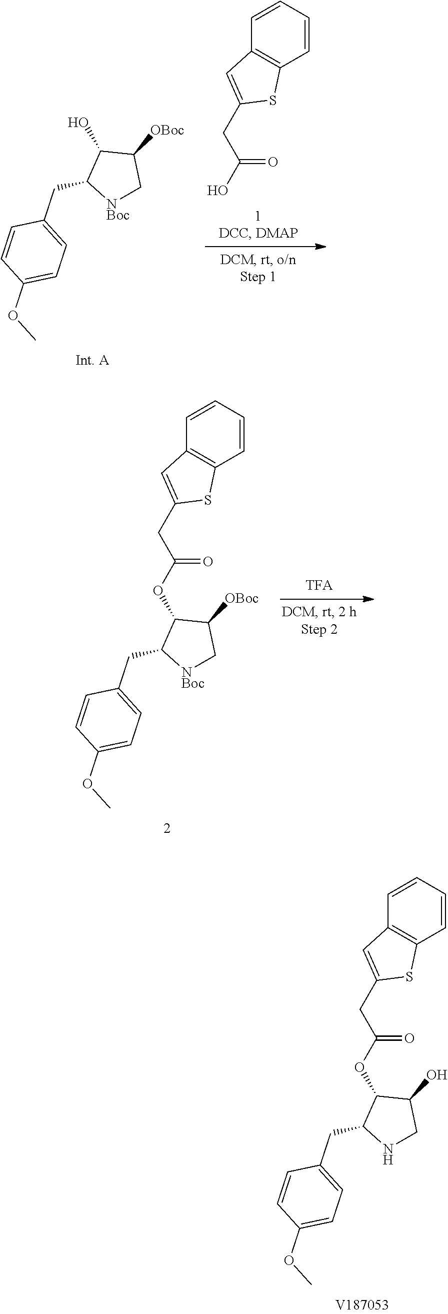

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 1-benzothiophen-2-ylacetic acid (90.7 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, the resulting mixture was filtered, the filter cake was washed with DCM (2×100 mL). The filtrate was concentrated under reduced pressure. The residue was purified by Prep-TLC (CH₂Cl₂/MeOH 10:1) to afford tert-butyl (2R,3S,4S)-3-{[2-(1-benzothiophen-2-yl)acetyl]oxy}-4-[(tert-butoxycarbonyl)oxy]-2-[(4-methoxyphenyl)methyl]pyrrolidine-1-carboxylate (80 mg, 56.6%) as a white solid. MS: m/z: Calc'd for C₃₂H₃₉NO₈S [M−H−56−56]⁺485, found 485.

Step 2: To a stirred solution of tert-butyl (2R,3S,4S)-3-{[2-(1-benzothiophen-2-yl)acetyl]oxy}-4-[(tert-butoxycarbonyl)oxy]-2-[(4-methoxyphenyl)methyl]pyrrolidine-1-carboxylate (80 mg, 0.13 mmol, 1 eq.) in DCM (3 mL) was added TFA (1 mL) dropwise at 0° C. The reaction was stirred at room temperature for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(1-benzothiophen-2-yl)acetate (28.4 mg, 53.3%) as a white solid. MS: m/z: Calc'd for C₂₂H₂₃NO₄S [M+H]⁺398, found 398. ¹H NMR (400 MHz, Methanol-d₄) δ 7.89-7.80 (m, 2H), 7.38-7.30 (m, 3H), 7.10-7.02 (m, 2H), 6.85-6.77 (m, 2H), 5.09 (d, J=3.5 Hz, 1H), 4.40 (d, J=4.2 Hz, 1H), 4.22-4.18 (m, 1H), 4.14 (d, J=3.2 Hz, 2H), 3.75 (s, 3H), 3.58-3.53 (m, 1H), 3.24 (d, J=12.6 Hz, 1H), 3.04-3.01 (m, 1H), 2.91-2.86 (m, 1H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 17% B to 47% B in 10 min; Wave Length: 254 nm/220 nm nm; RT1(min): 8.68

### Synthetic Procedure of V187054

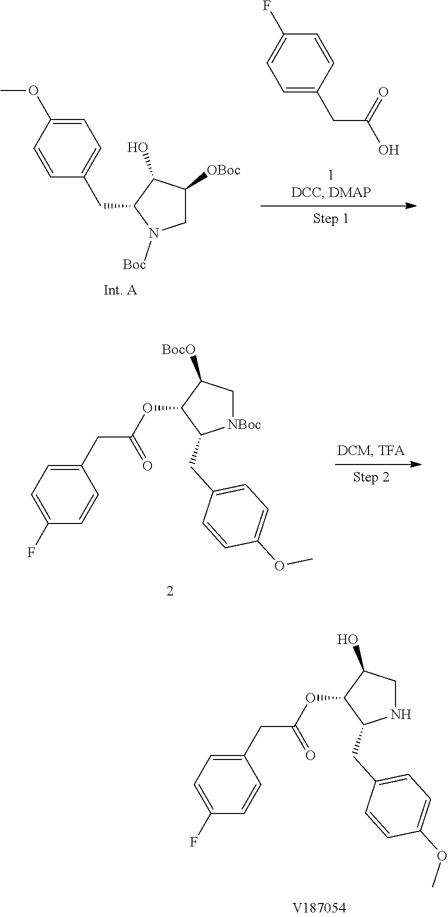

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and P-fluorophenylacetic acid (72.7 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phased-phase column chromatography (0.05% TFA in water and acetonitrile) to afford tert-butyl (2R,3S,4S)-4-[(tert-butoxycarbonyl)oxy]-3-{[2-(4-fluorophenyl)acetyl]oxy}-2-[(4-methoxyphenyl) methyl]pyrrolidine-1-carboxylate (65 mg, 49.1%) as a white solid. MS: m/z: Calc'd for C₃₀H₃₈FNO₈[M+H−56−56]⁺448, found 448.

Step 2: To a stirred mixture of tert-butyl (2R,3S,4S)-4-[(tert-butoxycarbonyl)oxy]-3-{[2-(4-fluorophenyl)acetyl]oxy}-2-[(4-methoxyphenyl)methyl]pyrrolidine-1-carboxylate (70 mg, 0.12 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(4-fluorophenyl) acetate (22.8 mg, 49.5%) as a white solid. MS: m/z: Calc'd for C₂₀H₂₂FNO₄ [M+H]⁺360; Found, 360. ¹H NMR (400 MHz, Methanol-d₄) δ 7.43-7.34 (m, 2H), 7.15-7.00 (m, 2H), 6.98-6.93 (m, 2H), 6.81-6.73 (m, 2H), 4.72-4.70 (m, 1H), 4.08-4.05 (m, 1H), 3.74 (d, J=13.4 Hz, 5H), 3.55-3.43 (m, 1H), 3.30 (d, J=5.8 Hz, 1H), 2.80-2.61 (m, 3H). Prep-HPLC-conditions: Column: XSelect CSH Prep C18 OBD Column, 5 μm, 30*150 mm; Mobile Phase A: Water (0.05% NH₄HCO₃), Mobile Phase B:ACN; Flow rate:60 mL/min; Gradient:35 B to 55 B in 10 min; 254/220 nm; RT1:8.18; RT2:; Injection Volumn: ml; Number Of Runs:;

### Synthetic Procedure of V187055

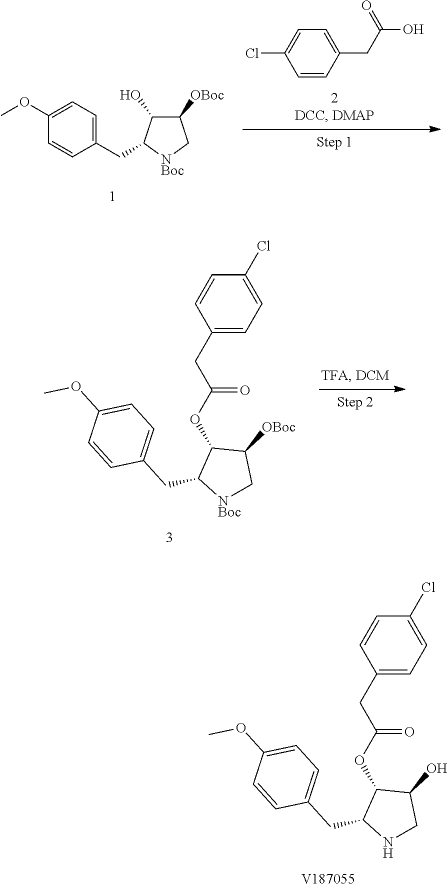

Step 1: To a stirred solution of (4-chlorophenyl)acetic acid (65 mg, 0.38 mmol, 2 eq) in DCM (5 mL) was added DCC (59 mg, 0.28 mmol, 1.5 eq), DMAP (23 mg, 0.19 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq) at 0° C. under nitrogen atmosphere. The solution was stirred at room temperature for 12 h. The mixture was concentrated and the residue was purified by reversed-phase flash chromatography to afford tert-butyl (2R,3S,4S)-4-[(tert-butoxycarbonyl)oxy]-3-{[2-(4-chlorophenyl)acetyl]oxy}-2-[(4-methoxyphenyl)methyl]pyrrolidine-1-carboxylate (86 mg, 79.0%) as a light yellow solid. MS: m/z: Calc'd for C₃₀H₃₈ClNO₈[M+H−56−56]⁺464, found 464.

Step 2: To a stirred solution of tert-butyl (2R,3S,4S)-4-[(tert-butoxycarbonyl)oxy]-3-{[2-(4-chlorophenyl)acetyl]oxy}-2-[(4-methoxyphenyl)methyl]pyrrolidine-1-carboxylate (86 mg, 0.15 mmol, 1 eq) in DCM (5 mL) was added TFA (1 mL) dropwise at 0° C. under nitrogen atmosphere. The solution was stirred at room temperature for 1 h. The mixture was concentrated and the crude product (60 mg) was purified by Prep-HPLC to afford (2R,3S,4S)-4-hydroxy-2-[(4-methoxyphenyl)methyl]pyrrolidin-3-yl 2-(4-chlorophenyl)acetate; trifluoroacetic acid (15.7 mg, 21.4%) as a white solid. MS: m/z: Calc'd for C₂₀H₂₂ClNO₄ [M+H]⁺376, found 376. ¹H NMR (400 MHz, Methanol-d₄) δ 7.45-7.32 (m, 4H), 7.10-7.04 (m, 2H), 6.88 (d, J=8.6 Hz, 2H), 5.08-5.03 (m, 1H), 4.35 (d, J=4.2 Hz, 1H), 4.23-4.18 (m, 1H), 3.81 (d, J=2.3 Hz, 2H), 3.79 (s, 3H), 3.53 (dd, J=12.6, 4.3 Hz, 1H), 3.23 (d, J=12.6 Hz, 1H), 2.99 (dd, J=14.3, 7.3 Hz, 1H), 2.87 (dd, J=14.2, 8.4 Hz, 1H). Prep-HPLC-conditions: Column: Xselect CSH Prep C18 Column, 30*150 mm, 5 μm; Mobile Phase A: Water(0.05% TFA), Mobile Phase B: ACN; Flow rate: 60 mL/min mL/min; Gradient: 4% B to 44% B in 10 min; Wave Length: 254 nm/220 nm nm; RT1(min): 8.68

### Synthetic Procedure of V187056

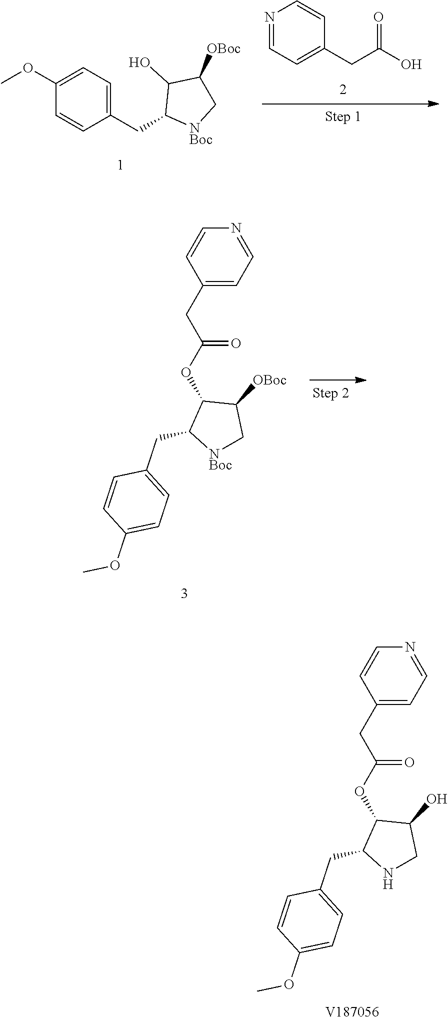

Step 1: To a stirred solution of tert-butyl (2R,4S)-4-[(tert-butoxycarbonyl)oxy]-3-hydroxy-2-[(4-methoxyphenyl)methyl]pyrrolidine-1-carboxylate (Int.A, 100 mg, 0.24 mmol, 1 eq.) and 4-acetylpyridine (64.8 mg, 0.47 mmol, 2 eq.) in DCM (8 mL) were added DCC (97.4 mg, 0.47 mmol, 2 eq.) and DMAP (43.3 mg, 0.35 mmol, 1.5 eq) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-{[2-(pyridin-4-yl)acetyl]oxy}pyrrolidine-1-carboxylate (70 mg, 54.6%) as a white solid. MS: m/z: Calc'd for C₂₉H₃₈N₂O₈ [M+H]⁺543, found 543.

Step 2: To a stirred solution of tert-butyl (2R,3S,4S)-4-[(tert-butoxycarbonyl)oxy]-2-[(4-methoxyphenyl)methyl]-3-{[2-(pyridin-4-yl)acetyl]oxy}pyrrolidine-1-carboxylate (60 mg, 0.11 mmol, 1 eq.) in DCM (5 mL) was added TFA (1 mL) at room temperature for 2 h. After completion of the reaction monitored by LCMS. Then the mixture was concentrated and purified by Prep-HPLC to obtain (2R,3S,4S)-4-hydroxy-2-[(4-methoxyphenyl)methyl]pyrrolidin-3-yl 2-(pyridin-4-yl)acetate; trifluoroacetic acid (14.1 mg, 27.9%) as a white solid. MS: m/z: Calc'd for C₁₉H₂₂N₂O₄ [M+H]⁺343, found 343. ¹H NMR (400 MHz, Methanol-d₄) δ 8.82 (s, 2H), 7.98 (s, 2H), 7.22 (d, J=8.1 Hz, 2H), 6.92 (d, J=8.0 Hz, 2H), 5.17 (d, J=3.5 Hz, 1H), 4.43 (d, J=4.2 Hz, 1H), 4.24 (d, J=5.1, 3.5 Hz, 1H), 3.79 (s, 3H), 3.64 (dd, J=12.9, 4.3 Hz, 1H), 3.30 (s, 1H), 3.25 (d, J=12.6 Hz, 1H), 3.09 (dd, J=14.4, 6.7 Hz, 1H), 2.99 (dd, J=14.4, 8.9 Hz, 1H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.5

### Synthetic Procedure of V187057

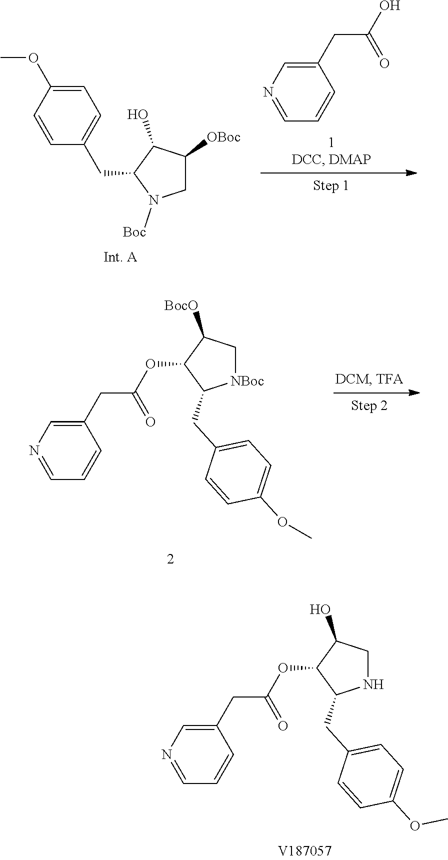

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-pyridylacetic acid (64.7 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl) methyl]-3-{[2-(pyridin-3-yl)acetyl]oxy}pyrrolidine-1-carboxylate (65 mg, 50.7%) as a white solid. MS: m/z: Calc'd for C₂₉H₃₈N₂O₈[M+H]⁺543, found 543.

Step 2: To a stirred mixture of tert-butyl (2R,3S,4S)-4-[(tert-butoxycarbonyl)oxy]-2-[(4-methoxyphenyl)methyl]-3-{[2-(pyridin-3-yl)acetyl]oxy}pyrrolidine-1-carboxylate (65 mg, 0.12 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(pyridin-3-yl)acetate (28.7 mg, 68.79%) as a white solid. MS: m/z: Calc'd for C₁₉H₂₂N₂O₄ [M+H]⁺343; Found, 343. ¹H NMR (400 MHz, Methanol-d₄) δ 8.58-8.53 (m, 1H), 8.49-8.46 (m, 1H), 7.87-7.85 (m, 1H), 7.47 (m, 1H), 7.09-6.95 (m, 2H), 6.84-6.74 (m, 2H), 4.79 (d, J=3.6 Hz, 1H), 4.16-4.10 (m, 1H), 3.95-3.76 (m, 5H), 3.57-3.55 (m, 1H), 3.36 (d, J=5.8 Hz, 1H), 2.80-2.65 (m, 3H). Prep-HPLC-conditions: Column: XSelect CSH Prep C18 OBD Column, 5 μm, 19*150 mm; Mobile Phase A:Water (0.05% TFA), Mobile Phase B:ACN; Flow rate:60 mL/min; Gradient:38 B to 50 B in 22 min; 254/220 nm; RT1:10.48; RT2:; Injection Volumn: ml; Number Of Runs:;

### Synthetic Procedure of V187061

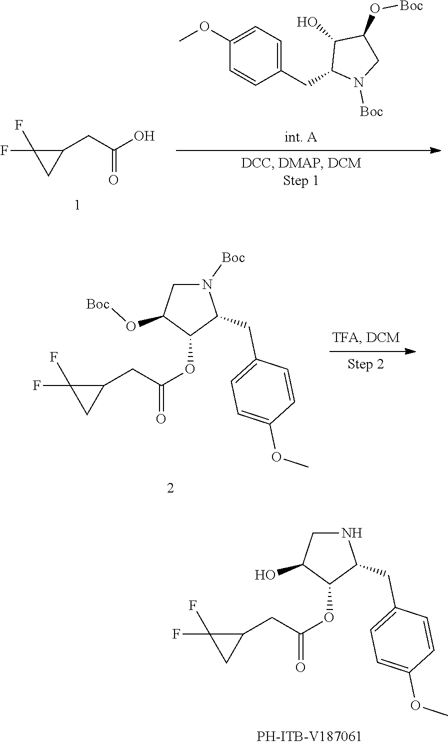 [SMILES: COc1ccc(C[C@@H]2[C@H](O)[C@@H](OC(=O)OC(C)(C)C)CN2C(=O)OC(C)(C)C)cc1 ; O=C(O)CC1CC1(F)F ; COc1ccc(C[C@@H]2[C@H](OC(=O)CC3CC3(F)F)[C@@H](OC(=O)OC(C)(C)C)CN2C(=O)OC(C)(C)C)cc1 ; COc1ccc(C[C@H]2NC[C@H](O)[C@H]2OC(=O)CC2CC2(F)F)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and (2,2-difluorocyclopropyl)acetic acid (51.4 mg, 0.37 mmol, 2 eq.) in DCM (5 mL) were added DCC (116.9 mg, 0.56 mmol, 3 eq.) and DMAP (34.6 mg, 0.28 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-{[2-(2,2-difluorocyclopropyl)acetyl]oxy}-2-[(4-methoxyphenyl)methyl]pyrrolidine-1-carboxylate (100 mg, 97.7%) as a yellow oil. MS: m/z: Calc'd for C₂₇H₃₇F₂NO₈[M+H−56−56]⁺430, found 430.

Step 2: To a stirred mixture of tert-butyl (2R,3S,4S)-4-[(tert-butoxycarbonyl)oxy]-3-{[2-(2,2-difluorocyclopropyl)acetyl]oxy}-2-[(4-methoxyphenyl)methyl]pyrrolidine-1-carboxylate (100 mg, 0.18 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford 2R,3S,4S)-4-hydroxy-2-[(4-methoxyphenyl)methyl]pyrrolidin-3-yl 2-(2,2-difluorocyclopropyl)acetate (31.6 mg, 49.6%) as a white semi-solid. MS: m/z: Calc'd for C₁₇H₂₁F₂NO₄ [M+H]⁺342, found 342. ¹H NMR (400 MHz, Methanol-d₄) δ 7.31-7.13 (m, 2H), 6.93-6.91 (m, 2H), 5.16-5.08 (m, 1H), 4.38-4.36 (m, 1H), 4.26-4.16 (m, 1H), 3.79-3.77 (m, 3H), 3.66-3.51 (m, 1H), 3.24 (d, J=12.8 Hz, 1H), 3.10-3.08 (m, 1H), 3.00 (d, J=9.9 Hz, 1H), 2.76-2.74 (m, 1H), 2.68-2.56 (m, 1H), 1.98-1.96 (m, 1H), 1.65-1.63 (m, 1H), 1.21-1.19 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 7% B to 37% B in 10 min; Wave Length: 254 nm/220 nm nm; RT1(min): 8.68.

### Synthetic Procedure of V187062

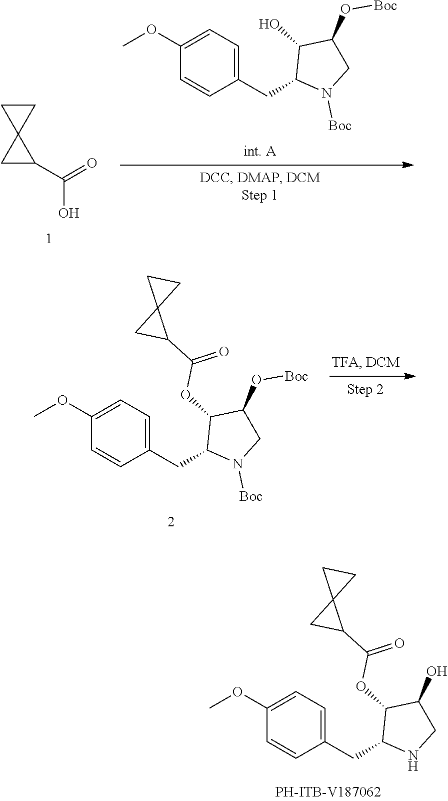 [SMILES: O=C(O)C1CC12CC2 ; COc1ccc(C[C@@H]2[C@H](O)[C@@H](OC(=O)OC(C)(C)C)CN2C(=O)OC(C)(C)C)cc1 ; COc1ccc(C[C@@H]2[C@H](OC(=O)C3CC34CC4)[C@@H](OC(=O)OC(C)(C)C)CN2C(=O)OC(C)(C)C)cc1 ; COc1ccc(C[C@H]2NC[C@H](O)[C@H]2OC(=O)C2CC23CC3)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and spiro[2.2]pentane-1-carboxylic acid (42.3 mg, 0.37 mmol, 2 eq.) in DCM (5 mL) were added DCC (116.9 mg, 0.56 mmol, 3 eq.) and DMAP (1.30 mg, 0.01 mmol, 1.5 eq.) at 0° C. under nitrogen atmosphere. The resulting reaction mixture was stirred at ambient temperature for overnight. Upon completion, the reaction mixture was concentrated in vacuo. The resulting residue The residue was purified by Prep-TLC (PE/EA 5:1) to afford tert-butyl (2R,3S,4S)-4-[(tert-butoxycarbonyl)oxy]-2-[(4-methoxyphenyl)methyl]-3-{spiro[2.2]pentane-1-carbonyloxy}pyrrolidine-1-carboxylate (100 mg, 98.2%) as a yellow oil. MS: m/z: Calc'd for C₂₈H₃₉NO₈[M+H−100]⁺418, found 418.

Step 2: To a stirred mixture of tert-butyl (2R,3S,4S)-4-[(tert-butoxycarbonyl)oxy]-2-[(4-methoxyphenyl)methyl]-3-{spiro[2.2]pentane-1-carbonyloxy}pyrrolidine-1-carboxylate (100 mg, 0.19 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl spiro[2.2]pentane-1-carboxylate (34.4 mg, 40.28%) as a white solid. MS: m/z: Calc'd for C₁₈H₂₃NO₄ [M+H]⁺318, found 318. ¹H NMR (400 MHz, Methanol-d₄) δ 7.19-7.17 (m, 2H), 6.95-6.87 (m, 2H), 4.88 (d, J=3.6 Hz, 1H), 4.21 (d, J=3.6 Hz, 1H), 4.04 (dd, J=7.8, 3.6 Hz, 1H), 4.02 (s, 4H), 3.94 (s, 3H), 3.77-3.75 (m, 1H), 3.42-3.40 (m, 2H), 3.13 (dd, J=16.0, 7.8 Hz, 2H), 2.61-2.54 (m, 1H), 2.52-2.46 (m, 1H), 2.49-2.41 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 6% B to 36% B in 10 min; Wave Length: 254 nm/220 nm nm; RT1(min): 8.68

### Synthetic Procedure of V187063

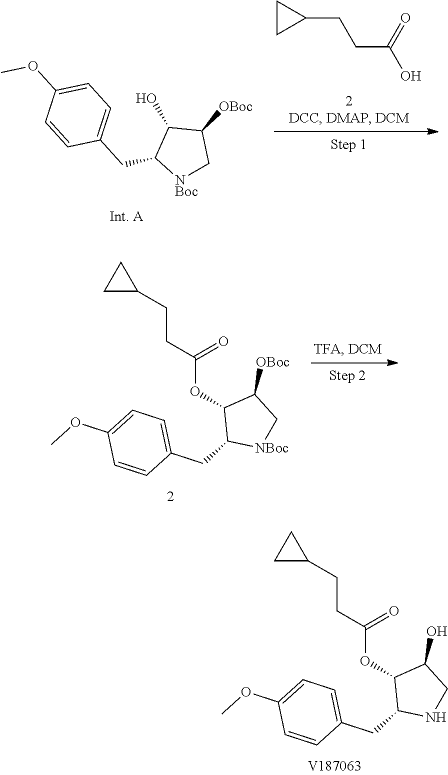

Step 1: A solution of 3-cyclopropylpropionic acid (43 mg, 0.37 mmol, 2.00 equiv) in DCM (5 mL) was treated with DCC (58.4 mg, 0.28 mmol, 1.50 equiv) and DMAP (23.1 mg, 0.19 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 equiv). The resulting mixture was stirred for overnight at room temperature under nitrogen atmosphere. The resulting mixture was filtered, the filter cake was washed with DCM (3*30 mL). The filtrate was concentrated under reduced pressure. The residue was purified by Prep-TLC (PE/EA 3:1) to afford tert-butyl (2R,3S,4S)-4-[(tert-butoxycarbonyl) oxy]-3-[(3-cyclopropylpropanoyl)oxy]-2-[(4-methoxyphenyl)methyl]pyrrolidine-1-carboxylate (56 mg, 57.1%) as a yellow oil. MS: m/z: Calc'd for C₂₈H₄₁NO₈[M+H−56−56]⁺408, found 408.

Step 2: To a stirred solution of tert-butyl (2R,3S,4S)-4-[(tert-butoxycarbonyl)oxy]-3-[(3-cyclopropylpropanoyl)oxy]-2-[(4-methoxyphenyl)methyl]pyrrolidine-1-carboxylate (75 mg, 0.14 mmol) in DCM (2.5 mL) was added TFA (0.5 mL) at 0° C. under air atmosphere. The resulting mixture was stirred for 60 mins at room temperature under air atmosphere. Desired product could be detected by LCMS. The mixture was concentrated. The crude product (50 mg) was purified by Prep-HPLC to afford (2R,3S,4S)-4-hydroxy-2-[(4-methoxyphenyl)methyl]pyrrolidin-3-yl 3-cyclopropylpropanoate; trifluoroacetic acid (20.9 mg, 32.9%) as a yellow solid. MS: m/z: Calc'd for C₁₈H₂₅NO₄ [M+H]⁺320, found 320. ¹H NMR (400 MHz, Methanol-d₄) δ 7.27-7.19 (m, 2H), 6.97-6.89 (m, 2H), 5.14-5.08 (m, 1H), 4.39-4.33 (m, 1H), 4.19-4.09 (m, 1H), 3.79 (s, 3H), 3.59 (dd, J=12.7, 4.4 Hz, 1H), 3.22 (d, J=12.6 Hz, 1H), 3.10 (dd, J=14.3, 6.7 Hz, 1H), 2.96 (dd, J=14.3, 8.9 Hz, 1H), 2.59-2.50 (m, 2H), 1.59-1.40 (m, 2H), 0.84-0.71 (m, 1H), 0.53-0.43 (m, 2H), 0.16-0.08 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 9% B to 39% B in 10 min; Wave Length: 254 nm/220 nm nm; RT1(min): 8.68

### Synthetic Procedure of V187064

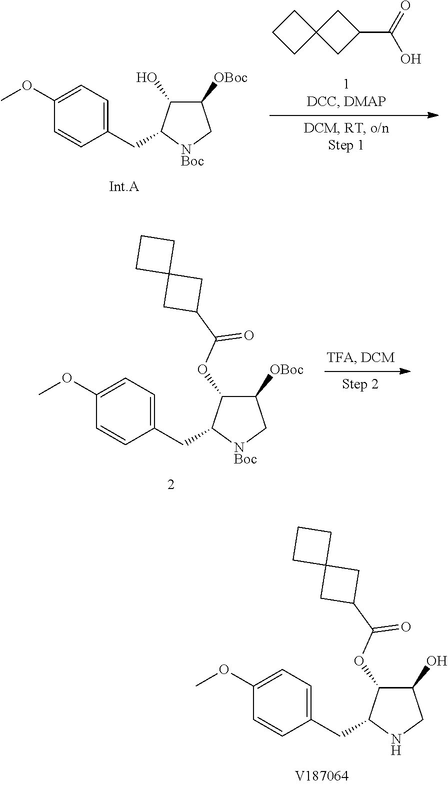

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and spiro[3.3]heptane-2-carboxylic acid (53.0 mg, 0.38 mmol, 2.0 eq) in DCM (3 mL) were added DCC (58.5 mg, 0.28 mmol, 1.5 eq.) and DMAP (23.1 mg, 0.19 mmol, 1.0 eq.) at 0° C. under nitrogen atmosphere. The resulting reaction mixture was stirred at ambient temperature for overnight. The reaction was purified by Prep-TLC (PE/EA 5:1) to afford tert-butyl (2R,3S,4S)-4-[(tert-butoxycarbonyl)oxy]-2-[(4-methoxyphenyl)methyl]-3-{spiro[3.3]heptane-2-carbonyloxy}pyrrolidine-1-carboxylate (60 mg, 58.21%) as a light yellow oil. MS: m/z: Calc'd for C₃₀H₄₃NO₈[M+H−56−100]⁺390, found 390.

Step 2: To a stirred mixture of tert-butyl (2R,3S,4S)-4-[(tert-butoxycarbonyl)oxy]-2-[(4-methoxyphenyl)methyl]-3-{spiro[3.3]heptane-2-carbonyloxy}pyrrolidine-1-carboxylate (50 mg, 0.09 mmol, 1.0 eq) in dioxane (1 mL) was added HCl(gas) in 1,4-dioxane (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-ethoxyphenyl)methyl]pyrrolidin-3-yl spiro[3.3]heptane-2-carboxylate (11.5 mg, 36.11%) as a white solid. MS: m/z: Calc'd for C₂₀H₂₇NO₄ [M+H]⁺346, found 346. ¹H NMR (400 MHz, Methanol-d₄) δ 7.12 (d, J=8.1 Hz, 2H), 6.85 (d, J=8.1 Hz, 2H), 4.79 (d, J=3.7 Hz, 1H), 4.13-4.07 (m, 1H), 3.78 (s, 3H), 3.60-3.57 (m, 1H), 3.13-3.07 (m, 1H), 2.77 (dd, J=14.0, 9.7 Hz, 3H), 2.38-2.31 (m, 1H), 2.28 (dd, J=10.9, 7.5 Hz, 3H), 2.12-2.05 (m, 2H), 2.00-1.92 (m, 2H), 1.89-1.83 (m, 2H). Prep-HPLC-conditions: Column: ACN; Mobile Phase A: 60 mL/min, Mobile Phase B: 29% B to 59% B in 8 min; Flow rate: 6.8 mL/min; Gradient: isocratic 3; Wave Length: Water (10 mmol/L NH₄HCO₃) nm; RT1(min): 24

### Synthetic Procedure of V187065

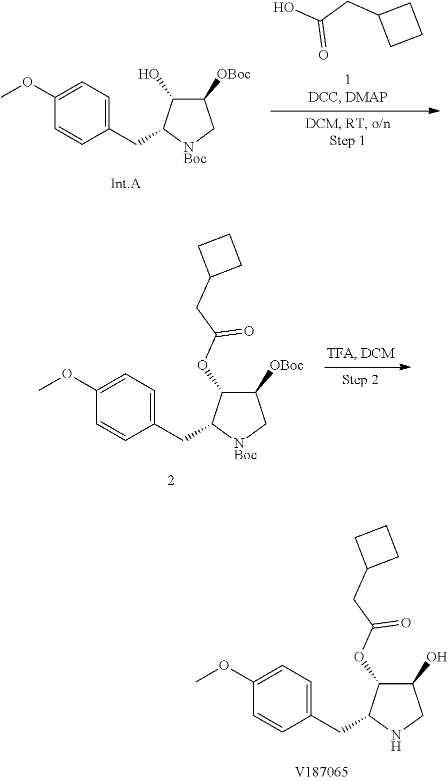

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq) and cyclobutylacetic acid (43.1 mg, 0.38 mmol, 2.0 eq) in DCM (5 mL) were added DCC (58.5 mg, 0.28 mmol, 1.5 eq.) and DMAP (23.08 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-[(2-cyclobutylacetyl)oxy]-2-[(4-methoxyphenyl)methyl]pyrrolidine-1-carboxylate (70 mg, 71.3%) as a light yellow oil. MS: m/z: Calc'd for C₂₈H₄₁NO₈[M+H−56−100]⁺364, found 364.

Step 2: To a stirred mixture of tert-butyl (2R,3S,4S)-4-[(tert-butoxycarbonyl)oxy]-3-[(2-cyclobutylacetyl)oxy]-2-[(4-methoxyphenyl)methyl]pyrrolidine-1-carboxylate (65 mg, 0.13 mmol, 1.0 eq) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford to afford (2R,3S,4S)-4-hydroxy-2-[(4-methoxyphenyl)methyl]pyrrolidin-3-yl 2-cyclobutylacetate (13.1 mg, 32.7%) as a light yellow solid. MS: m/z: Calc'd for C₁₈H₂₅NO₄ [M+H]⁺320, found 320. ¹H NMR (400 MHz, Methanol-d4) δ 7.23 (d, J=8.2 Hz, 2H), 6.99-6.87 (m, 2H), 5.09 (d, J=3.4 Hz, 1H), 4.32 (d, J=4.3 Hz, 1H), 4.25-4.14 (m, 1H), 3.80 (s, 3H), 3.59 (dd, J=13.3, 4.6 Hz, 1H), 3.23 (d, J=12.7 Hz, 1H), 3.08 (dd, J=14.3, 6.9 Hz, 1H), 2.96 (dd, J=14.3, 8.7 Hz, 1H), 2.77-2.75 (m, 1H), 2.60 (d, J=7.6 Hz, 2H), 2.24-2.16 (m, 2H), 2.04-1.88 (m, 2H), 1.86-1.76 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 11% B to 41% B in 10 min; Wave Length: 254 nm/220 nm nm; RT1(min): 8.68

### Synthetic Procedure of V187066

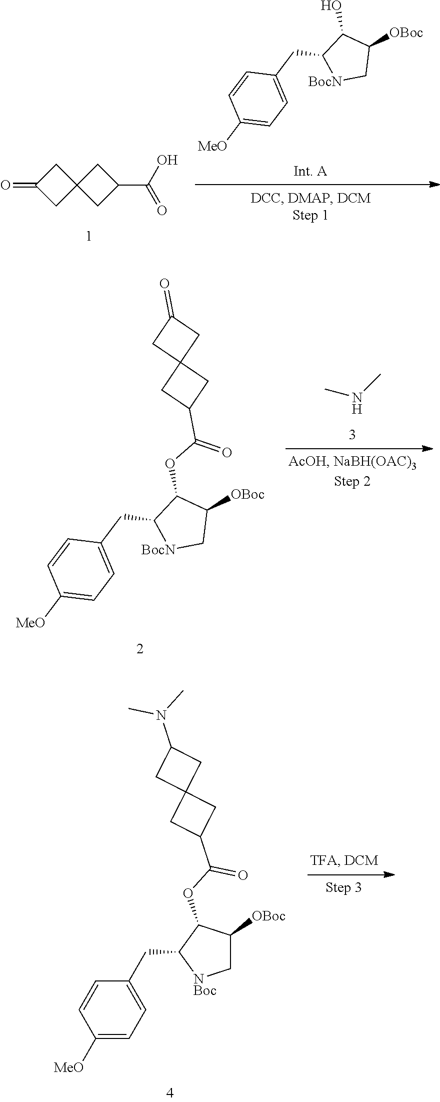

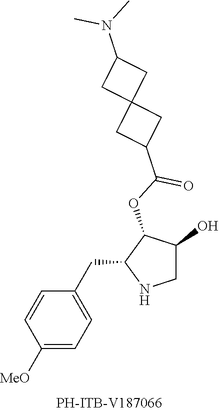 [SMILES: COc1ccc(C[C@H]2NC[C@H](O)[C@H]2OC(=O)C2CC3(C2)CC(N(C)C)C3)cc1]

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 274.7 mg, 0.64 mmol, 1.00 eq.) and 6-oxospiro[3.3]heptane-2-carboxylic acid (200 mg, 1.29 mmol, 2 eq.) in DCM (15 mL) were added DCC (401.5 mg, 1.94 mmol, 3 eq.) and DMAP (118.87 mg, 0.973 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved in DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-{6-oxospiro[3.3]heptane-2-carbonyloxy}pyrrolidine-1-carboxylate (260 mg, 71.6%) as a yellow oil. MS: m/z: Calc'd for C₃₀H₄₁NO₉ [M+H−100]⁺460, found 460.

Step 2: To a solution of tert-butyl (2R,3S,4S)-4-[(tert-butoxycarbonyl)oxy]-2-[(4-methoxyphenyl)methyl]-3-{6-oxospiro[3.3]heptane-2-carbonyloxy}pyrrolidine-1-carboxylate (260 mg, 0.46 mmol, 1.0 eq.) in DCM (15 mL) were added AcOH (55.8 mg, 0.93 mmol, 2.0 eq.) and dimethylamine (2 M in THF) (62.8 mg, 1.39 mmol, 3.0 eq.). The mixture was stirred at room temperature for 1 hours, followed by the addition of NaBH(OAC)₃ (196.9 mg, 0.93 mmol, 2 eq.) in portions at room temperature. The resulting mixture was stirred at room temperature for another 2 h. Upon completion, the reaction mixture was concentrated in vacuo. The crude product was used in the next step directly without further purification. MS: m/z: Calc'd for C₃₂H₄₈N₂O₈ [M+H−56]⁺533, found 533.

Step 3: To a stirred mixture of tert-butyl (2R,3S,4S)-4-[(tert-butoxycarbonyl)oxy]-3-[6-(dimethylamino)spiro[3.3]heptane-2-carbonyloxy]-2-[(4-methoxyphenyl)methyl]pyrrolidine-1-carboxylate (80 mg, 0.13 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 6-(dimethylamino)spiro[3.3]heptane-2-carboxylate (13.1 mg, 23.6%) as a yellow solid. MS: m/z: Calc'd for C₂₂H₃₂N₂O₄ [M+H]⁺389, found 389. ¹H NMR (400 MHz, Methanol-d₄) δ 7.16-7.05 (m, 2H), 6.89-6.78 (m, 2H), 4.79-4.77 (m, 1H), 4.09-4.07 (m, 1H), 3.77 (s, 3H), 3.56-3.54 (m, 1H), 3.36 (s, 1H), 3.20-3.14 (m, 1H), 2.81-2.68 (m, 3H), 2.62-2.60 (m, 1H), 2.44-2.20 (m, 5H), 2.14 (s, 7H), 1.88-1.84 (m, 2H). Prep-HPLC-conditions: Column: XBridge Prep Shield RP C18 Column, 19*250 mm, 5 μm; Mobile Phase A: Water (10 mmol/L NH₄HCO₃), Mobile Phase B: ACN; Flow rate: 25 mL/min mL/min; Gradient: 21% B to 31% B in 12 min; Wave Length: 254 nm/220 nm nm; RT1(min): 11.73

### Synthetic Procedure of V187067

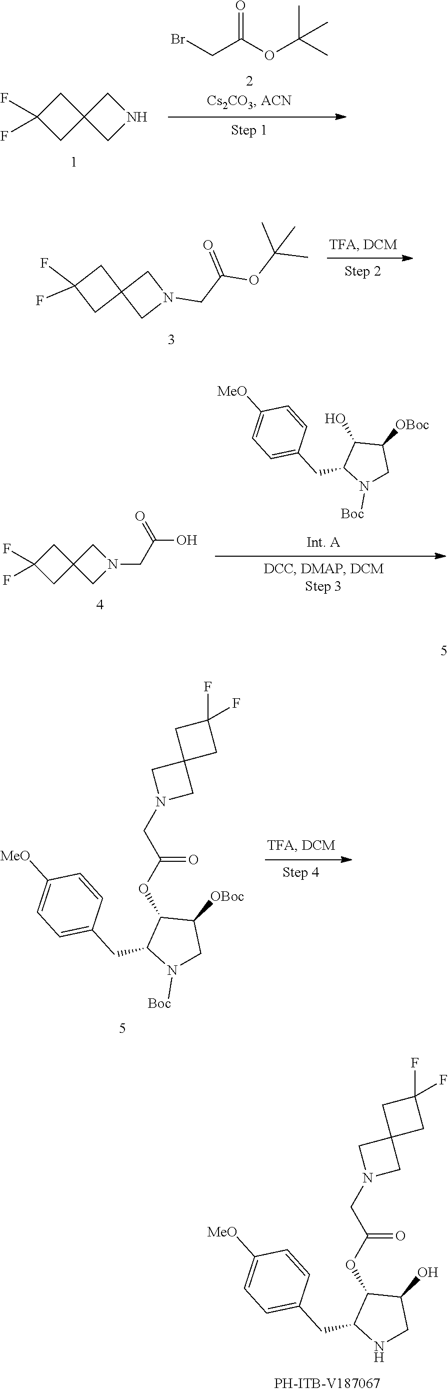

Step 1: To a stirred solution of tert-butyl 2-bromoacetate (118.4 mg, 0.60 mmol, 1.5 eq.) and 6,6-difluoro-2-azaspiro[3.3]heptane (100 mg, 0.40 mmol, 1.0 eq.) in ACN (10 mL) was added Cs₂CO₃ (395.7 mg, 1.21 mmol, 3 eq.) at room temperature. The mixture was stirred overnight at the same temperature. Upon completion, the reaction mixture was concentrated in vacuo. The resulting residue was dissolved with DMSO and purified by a reversed-phase column chromatography (0.05% TFA in water and acetonitrile) to afford tert-butyl 2-{6,6-difluoro-2-azaspiro[3.3]heptan-2-yl}acetate (150 mg, 81.0%) as a yellow oil. MS: m/z: Calc'd for C₁₂H₁₉F₂NO₂ [M+H]⁺248, found 248.

Step 2: To a solution of tert-butyl 2-{6,6-difluoro-2-azaspiro[3.3]heptan-2-yl}acetate (100 mg, 0.40 mmol, 1 eq.) in TFA (3 mL) and DCM (9 mL) was stirred for 2 hours at RT. Upon completion, the reaction mixture was concentrated in vacuo. The crude product was used in the next step directly without further purification. MS: m/z: Calc'd for C₈H₁₁F₂NO₂ [M+H]⁺192, found 192.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 70 mg, 0.165 mmol, 1 eq.) and {6,6-difluoro-2-azaspiro[3.3]heptan-2-yl}acetic acid (189.6 mg, 0.99 mmol, 6.00 eq.) in DCM (5 mL) were added DCC (102.3 mg, 0.49 mmol, 3.00 eq.) and DMAP (30.2 mg, 0.24 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved in DMSO and purified by a reversed-phase column chromatography (0.05% TFA in water and acetonitrile) to afford tert-butyl (2R,3S,4S)-4-[(tert-butoxycarbonyl)oxy]-3-[(2-{6,6-difluoro-2-azaspiro[3.3]heptan-2-yl}acetyl)oxy]-2-[(4-methoxyphenyl)methyl]pyrrolidine-1-carboxylate (90 mg, 91.8%) as a yellow oil. MS: m/z: Calc'd for C₃₀H₄₂F₂N₂O₈[M+H]⁺597, found 597.

Step 4: To a stirred mixture of (2R,3S,4S)-4-[(tert-butoxycarbonyl)oxy]-3-[(2-{6,6-difluoro-2-azaspiro[3.3]heptan-2-yl}acetyl)oxy]-2-[(4-methoxyphenyl)methyl]pyrrolidine-1-carboxylate (80 mg, 0.13 mmol) in 1,4-dioxane (1 mL) was added HC 1 (gas) in 1,4-dioxane (5 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-{6,6-difluoro-2-azaspiro[3.3]heptan-2-yl}acetate (22.8 mg, 42.7%) as a white solid. MS: m/z: Calc'd for C₂₀H₂₆F₂N₂O₄[M+H]⁺397, found 397. ¹H NMR (400 MHz, Methanol-d₄) δ 7.29-7.21 (m, 2H), 6.98-6.89 (m, 2H), 5.24 (d, J=3.8 Hz, 1H), 4.48-4.22 (m, 8H), 3.80 (s, 3H), 3.63-3.61 (m, 1H), 3.24-3.22 (m, 1H), 3.10-3.08 (m, 1H), 3.06-2.82 (m, 5H). Prep-HPLC-conditions: Column: Column: Xselect CSH Prep C18 C18 Column, 30*150 mm, 5 μm; Mobile Phase A: Water (0.1% FA), Mobile Phase B: ACN; Flow rate: 60 mL/min mL/min; Gradient: 2% B to 25% B in 10 min; Wave Length: 254 nm/220 nm nm; RT1(min): 8.68

### Synthetic Procedure of V187069

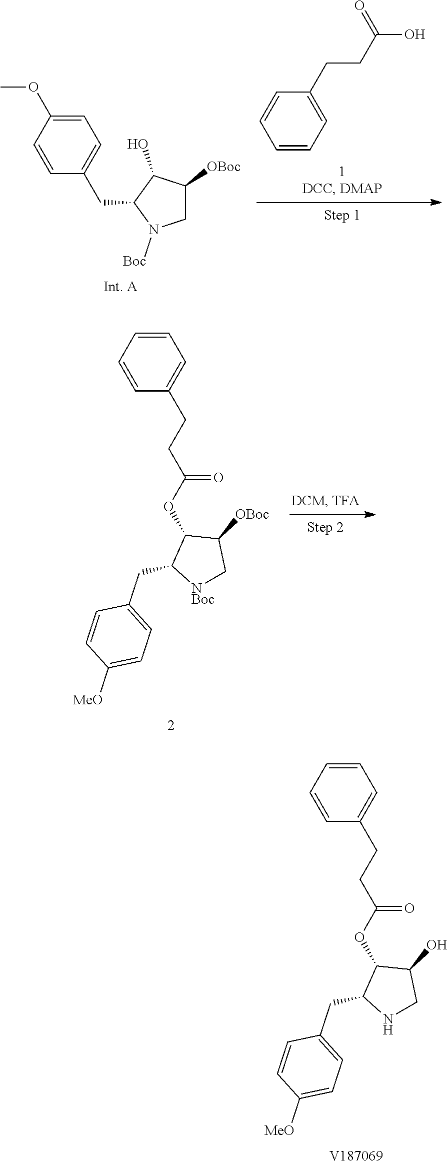

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-phenylpropionic acid (70.9 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) dropwise at 0° C. under nitrogen atmosphere. The resulting reaction mixture was stirred at ambient temperature for overnight. Upon completion, the reaction mixture was concentrated in vacuo. The resulting residue was purified by Prep-TLC to obtain tert-butyl (2R,3S,4S)-3-{[2-(2,1,3-benzoxadiazol-5-ylmethoxy) acetyl]oxy}-4-[(tert-butoxycarbonyl)oxy]-2-[(4-methoxyphenyl)methyl]pyrrolidine-1-carboxylate as a white solid. MS: m/z: Calc'd for C₃₁H₄₁NO₈[M+H]⁺556, found 556.

Step 2: To a stirred mixture of tert-butyl (2R,3S,4S)-4-[(tert-butoxycarbonyl)oxy]-2-[(4-methoxyphenyl)methyl]-3-[(3-phenylpropanoyl)oxy]pyrrolidine-1-carboxylate (60 mg, 0.10 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl 3-phenylpropanoate (15.6 mg, 40.65%) as a white solid. MS: m/z: Calc'd for C₂₁H₂₅NO₄ [M+H]⁺356; Found, 356. ¹H NMR (400 MHz, DMSO-d₆) δ 7.28-7.15 (m, 5H), 7.05-6.98 (m, 2H), 6.82-6.74 (m, 2H), 5.05 (d, J=4.3 Hz, 1H), 4.61-4.59 (m, 1H), 3.81-3.79 (m, 1H), 3.70 (s, 3H), 3.27-3.25 (m, 1H), 3.10-3.08 (m, 1H), 2.89 (t, J=6.9 Hz, 2H), 2.71 (t, J=7.1 Hz, 2H), 2.51-2.43 (m, 3H), 2.24 (s, 1H). Prep-HPLC-conditions: Column: XSelect CSH Prep C18 OBD Column, 5 μm, 30*150 mm; Mobile Phase A:Water (0.05% NH₄HCO₃, Mobile Phase B:ACN; Flow rate:60 mL/min; Gradient: 27 B to 57 B in 11 min; 254/220 nm; RT1:6.1; RT2:; Injection Volumn: ml; Number Of Runs:;

### Synthetic Procedure of V187070

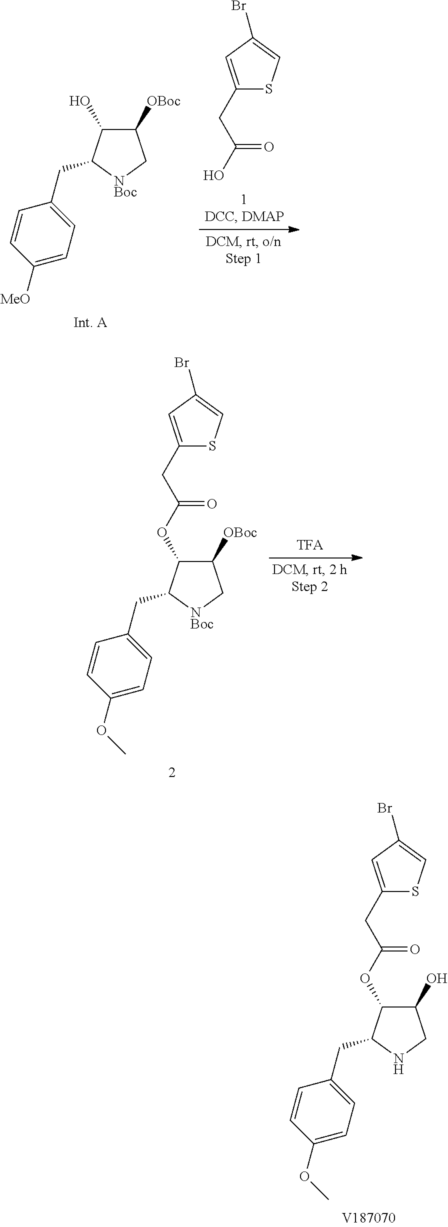

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and (4-bromothiophen-2-yl)acetic acid (104.4 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) dropwise at 0° C. The reaction was stirred at r.t. for overnight. After completion of the reaction monitored by LCMS, the resulting mixture was filtered, the filter cake was washed with DCM (1×100 mL). The filtrate was concentrated under reduced pressure. The residue was purified by Prep-TLC (CH₂Cl₂/MeOH 10:1) to afford tert-butyl (2R,3S,4S)-3-{[2-(4-bromothiophen-2-yl)acetyl]oxy}-4-[(tert-butoxycarbonyl)oxy]-2-[(4-methoxyphenyl)methyl]pyrrolidine-1-carboxylate (80 mg, 54.0%) as a white solid. MS: m/z: Calc'd for C₂₈H₃₆BrNO₈S [M−H−56−56]⁺513, found 513.

Step 2: To a stirred solution of tert-butyl (2R,3S,4S)-3-{[2-(4-bromothiophen-2-yl)acetyl]oxy}-4-[(tert-butoxycarbonyl)oxy]-2-[(4-methoxyphenyl)methyl]pyrrolidine-1-carboxylate (75 mg, 0.12 mmol, 1 eq.) in DCM (3 mL) was added TFA (1 mL) dropwise at 0° C. The reaction was stirred at r.t. for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(4-bromothiophen-2-yl)acetate (22.9 mg, 44.8%) as a white solid. MS: m/z: Calc'd for C₁₈H₂₀BrNO₄S [M+H]⁺426, found 426.

¹H NMR (400 MHz, Methanol-d₄) δ 7.38 (d, J=1.5 Hz, 1H), 7.17-7.09 (m, 2H), 7.04 (t, J=1.2 Hz, 1H), 6.94-6.86 (m, 2H), 5.11-5.06 (m, 1H), 4.38 (d, J=4.3 Hz, 1H), 4.22-4.18 (m, 1H), 4.12-3.98 (m, 2H), 3.79 (s, 3H), 3.58-3.54 (m, 1H), 3.27-3.24 (m, 1H), 3.05-3.01 (m, 1H), 2.99-2.89 (m, 1H). Prep-HPLC-conditions: Column: UV 254 nm/220 nm Xselect CSH Prep C18 C18 Column, 30*150 mm, 5 μm Water(0.05% TFA) ACN 60 mL/min 8% B to 38% B in 10 min 8.68

### Synthetic Procedure of V187072

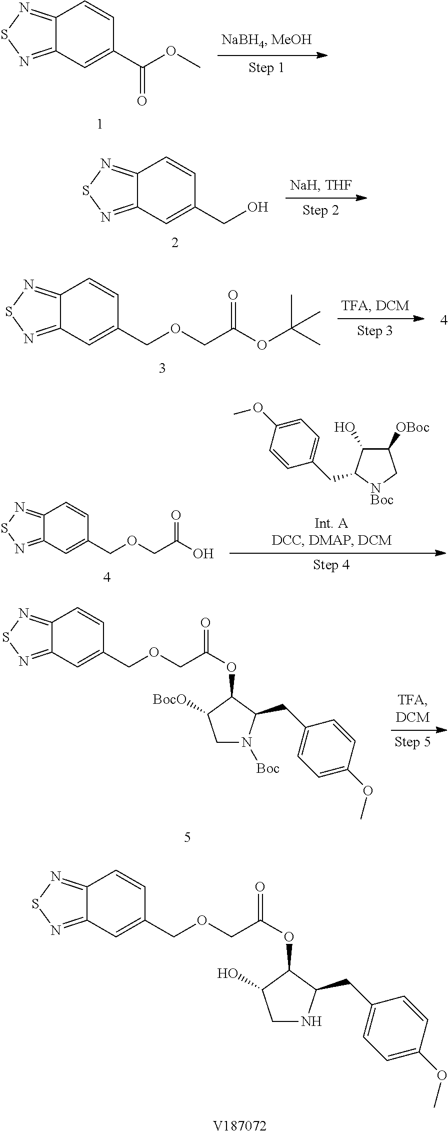

Step 1: To a stirred solution of methyl 2,1,3-benzothiadiazole-5-carboxylate (200 mg, 1.03 mmol, 1 eq) in MeOH (10 mL) and H₂O (5 mL) was added NaBH₄ (117 mg, 3.09 mmol, 3.00 eq) slowly at 0° C. under nitrogen atmosphere. The resulting mixture was stirred at 0° C. for 2 h. The mixture was concentrated and the residue was purified by reversed-phase flash chromatography to afford 2,1,3-benzothiadiazol-5-ylmethanol (230 mg, 134.38%) as a colorless solid. MS: m/z: Calc'd for C₇H₆N₂OS [M+H]⁺167, found 167.

Step 2: A solution of 2,1,3-benzothiadiazol-5-ylmethanol (220 mg, 1.32 mmol, 1 e) in THF (15 mL) was treated with NaH (95 mg, 3.97 mmol, 3 eq) at 0° C. for 30 min under nitrogen atmosphere followed by the addition of tert-butyl 2-bromoacetate (387 mg, 1.99 mmol, 1.5 eq). The resulting mixture was stirred at 0° C. for 2 h. The reaction was quenched with water. The aqueous layer was extracted with EA, dried and concentrated. The residue was purified by reversed-phase flash chromatography with the following conditions: column, C18 silica gel; mobile phase, ACN in Water (0.5% TFA), 10% to 60% gradient in 15 min; detector, UV 254 nm. This resulted in tert-butyl 2-(2,1,3-benzothiadiazol-5-ylmethoxy)acetate (95 mg, 25.6%) as a white solid. MS: m/z: Calc'd for C₁₃H₁₆N₂O₃S [M+H−56]⁺225, found 225.

Step 3: To a stirred solution of tert-butyl 2-(2,1,3-benzothiadiazol-5-ylmethoxy)acetate (95 mg, 0.34 mmol, 1 eq) in DCM (5 mL) was added TFA (1 mL) dropwise at 0° C. The resulting mixture was stirred at room temperature for 2 h. The resulting mixture was concentrated under reduced pressure. This resulted in (2,1,3-benzothiadiazol-5-ylmethoxy)acetic acid (75 mg, 98.7%) as a light yellow oil. MS: m/z: Calc'd for C₉H₈N₂O₃S [M+H]⁺225, found 225.

Step 4: To a stirred solution of (2,1,3-benzothiadiazol-5-ylmethoxy)acetic acid (64 mg, 0.28 mmol, 1.5 eq) in DCM (5 mL) was added DCC (58 mg, 0.28 mmol, 1.5 eq), DMAP (23 mg, 0.19 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq) at 0° C. under nitrogen atmosphere. The resulting mixture was stirred at room temperature for 12 h. The mixture was concentrated and the residue was purified by reversed-phase flash chromatography to afford tert-butyl (2R,3S,4S)-3-{[2-(2,1,3-benzothiadiazol-5-ylmethoxy)acetyl]oxy}-4-[(tert-butoxycarbonyl)oxy]-2-[(4-methoxyphenyl)methyl]pyrrolidine-1-carboxylate (95 mg, 79.9%) as a light yellow solid. MS: m/z: Calc'd for C₃₁H₃₉N₃O₉S [M+H−100]⁺530, found 530.

Step 5: To a stirred solution of tert-butyl (2R,3S,4S)-3-{[2-(2,1,3-benzothiadiazol-5-ylmethoxy)acetyl]oxy}-4-[(tert-butoxycarbonyl)oxy]-2-[(4-methoxyphenyl)methyl]pyrrolidine-1-carboxylate (90 mg, 0.14 mmol, 1 eq) in DCM (5 mL) was added TFA (1 mL) dropwise at 0° C. The resulting mixture was stirred at room temperature for 2 h. The mixture was concentrated and the crude product (70 mg) was purified by Prep-HPLC to afford (2R,3S,4S)-4-hydroxy-2-[(4-methoxyphenyl)methyl]pyrrolidin-3-yl 2-(2,1,3-benzothiadiazol-5-ylmethoxy)acetate; trifluoroacetic acid (35.2 mg, 44.6%) as a white solid. MS: m/z: Calc'd for C₂₁H₂₃N₃O₅S [M+H]⁺330, found 330. ¹H NMR (400 MHz, Methanol-d₄) δ 8.08-8.00 (m, 2H), 7.74-7.68 (m, 1H), 7.26-7.17 (m, 2H), 6.97-6.88 (m, 2H), 5.23-5.17 (m, 1H), 4.88 (d, J=1.1 Hz, 2H), 4.43 (dd, J=6.7, 4.5 Hz, 3H), 4.29-4.19 (m, 1H), 3.79 (s, 3H), 3.59 (dd, J=12.7, 4.4 Hz, 1H), 3.23 (d, J=12.8 Hz, 1H), 3.09 (dd, J=14.3, 6.7 Hz, 1H), 2.95 (dd, J=14.3, 8.9 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 12% B to 42% B in 10 min; Wave Length: 254 nm/220 nm nm; RT1(min): 8.68

### Synthetic Procedure of V187082

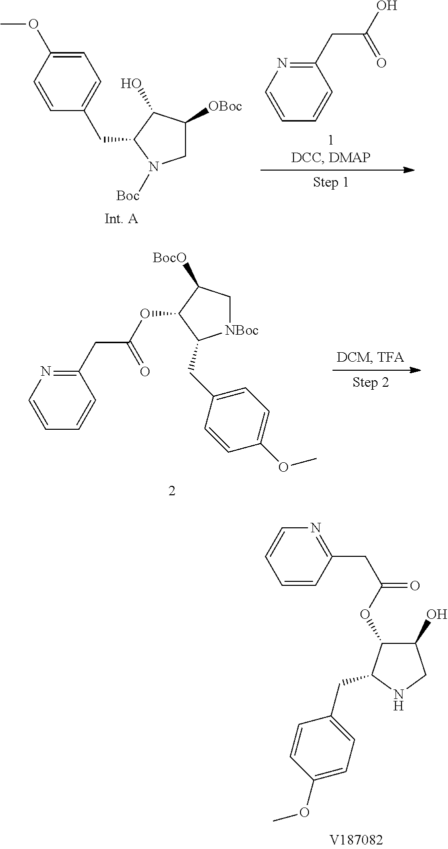

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and pyridin-2-ylacetic acid (64.7 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was purified by Prep-TLC to obtain tert-butyl (2R,3S,4S)-4-[(tert-butoxycarbonyl)oxy]-2-[(4-methoxyphenyl)methyl]-3-{[2-(pyridin-2-yl)acetyl]oxy}pyrrolidine-1-carboxylate (65 mg, 50.7%) as a white solid. MS: m/z: Calc'd for C₂₉H₃₈N₂O₈[M+H]⁺543, found 543.

Step 2: To a stirred mixture of tert-butyl (2R,3S,4S)-4-[(tert-butoxycarbonyl)oxy]-2-[(4-methoxyphenyl)methyl]-3-{[2-(pyridin-2-yl)acetyl]oxy}pyrrolidine-1-carboxylate (65 mg, 0.12 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(pyridin-2-yl)acetate (20.4 mg, 49.5%) as a white solid. MS: m/z: Calc'd for C₁₉H₂₂N₂O₄ [M+H]⁺343; Found, 343. ¹H NMR (400 MHz, Methanol-d₄) δ5.83-8.44 (m, 1H), 7.87-7.85 (m, 1H), 7.51 (d, J=7.8 Hz, 1H), 7.38-7.36 (m, 1H), 7.01 (d, J=7.9 Hz, 2H), 6.92-6.67 (m, 2H), 4.80 (d, J=3.6 Hz, 2H), 4.22-4.04 (m, 1H), 3.76 (s, 3H), 3.54 (t, J=3.6 Hz, 1H), 3.37 (d, J=6.0 Hz, 2H), 2.82-2.62 (m, 3H). Prep-HPLC-conditions: Column: XSelect CSH Prep C18 OBD Column, 5 μm, 30*150 mm; Mobile Phase A: Water (0.05% NH₄HCO₃), Mobile Phase B:ACN; Flow rate:60 mL/min; Gradient: 15 B to 45 B in 22 min; 254/220 nm; RT1:6.1; RT2:; Injection Volumn: ml; Number Of Runs:;

### Synthetic Procedure of V187088

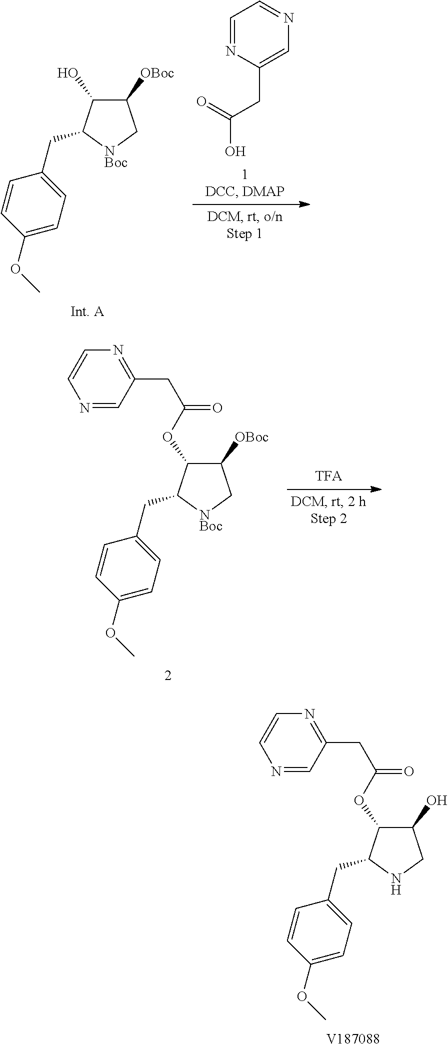

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.01 mmol, 1 eq.) and pyrazin-2-ylacetic acid (65.2 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at room temperature for overnight. After completion of the reaction monitored by LCMS, the mixture was purified directly by Prep-TLC (PE/EA 5:1) to afford tert-butyl (2R,3S,4S)-4-[(tert-butoxycarbonyl)oxy]-2-[(4-methoxyphenyl)methyl]-3-{[2-(pyrazin-2-yl)acetyl]oxy}pyrrolidine-1-carboxylate (80 mg, 62.3%) as a white solid. MS: m/z: Calc'd for C₂₈H₃₇N₃O₈[M−H]⁺544, found 544.

Step 2: To a stirred solution of tert-butyl (2R,3S,4S)-4-[(tert-butoxycarbonyl)oxy]-2-[(4-methoxyphenyl)methyl]-3-{[2-(pyrazin-2-yl)acetyl]oxy}pyrrolidine-1-carboxylate (75 mg, 0.13 mmol, 1 eq.) in DCM (3 mL) was added TFA (1 mL) dropwise at 0° C. The reaction was stirred at room temperature for 2 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 2-(pyrazin-2-yl)acetate (25.9 mg, 54.5%) as a yellow solid. MS: m/z: Calc'd for C₁₈H₂₁N₃O₄ [M+H]⁺344, found 344. ¹H NMR (400 MHz, Methanol-d₄) δ 8.71 (s, 1H), 8.66-8.56 (m, 2H), 7.20 (d, J=8.2 Hz, 2H), 6.92-6.89 (m, 2H), 5.13 (d, J=3.7 Hz, 1H), 4.43 (d, J=4.3 Hz, 1H), 4.21-4.19 (m, 1H), 4.18-4.07 (m, 1H), 3.82-3.77 (m, 3H), 3.64-3.54 (m, 1H), 3.32-3.20 (m, 2H), 3.05-3.02 (m, 1H), 2.95 (t, J=11.2 Hz, 1H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 2% B to 28% B in 10 min; Wave Length: 254 nm/220 nm nm; RT1(min): 8.68

### Synthetic Procedure of V187096

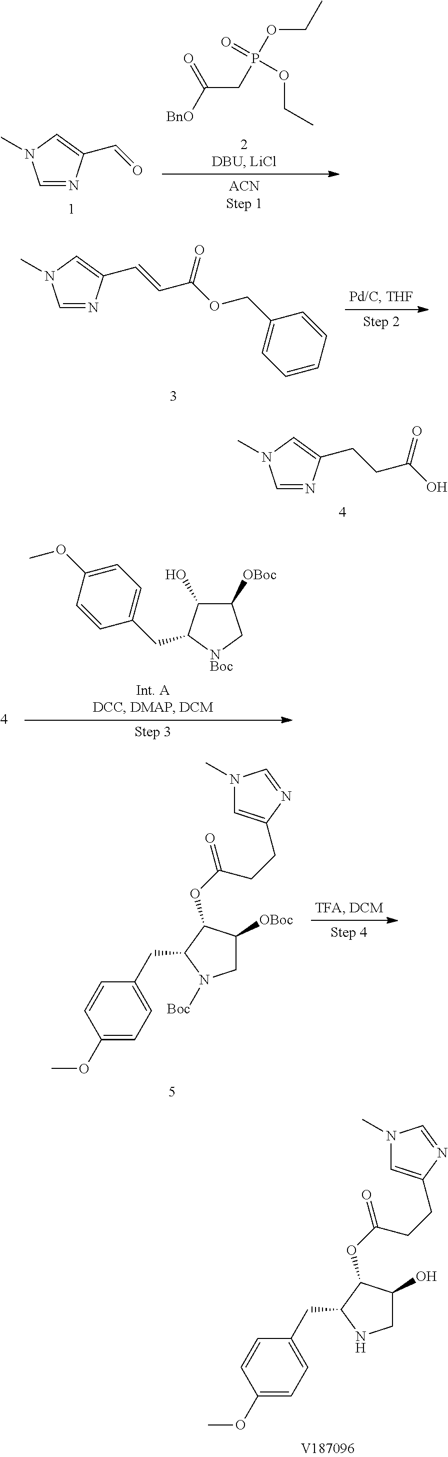

Step 1: A solution of benzyl 2-(diethoxyphosphoryl)acetate (1.04 g, 3.63 mmol, 2 eq) in ACN (10 mL) was treated with DBU (332 mg, 2.18 mmol, 1.2 eq) and LiCl (108 mg, 2.54 mmol, 1.4 eq) for 30 min at 0° C. under nitrogen atmosphere followed by the addition of 1-methylimidazole-4-carbaldehyde (200 mg, 1.82 mmol, 1 eq). The resulting mixture was stirred at room temperature for 12 h and concentrated to give the residue, which was applied onto a silica gel column eluting with ethyl acetate/petroleum ether (1:10). This resulted in benzyl (2E)-3-(1-methylimidazol-4-yl)prop-2-enoate (400 mg, 90.9%) as a light yellow solid. MS: m/z: Calc'd for C₁₄H₁₄N₂O₂ [M+H]⁺230; Found, 230.

Step 2: To a stirred solution of benzyl (2E)-3-(1-methylimidazol-4-yl)prop-2-enoate (400 mg, 1.65 mmol, 1 eq) in THF (15 mL) was added Pd/C (150 mg, 1.41 mmol, 0.9 eq). The flask was evacuated and flushed three times with nitrogen, followed by flushing with hydrogen. The mixture was stirred for 2 h at room temperature under an atmosphere of hydrogen (pressure tank reactor 10 atm). The resulting mixture was filtered, the filter cake was washed with MeOH (2×20 mL). The filtrate was concentrated under reduced pressure. This resulted in 3-(1-methylimidazol-4-yl)propanoic acid (150 mg, 58.93%) as a colorless solid. MS: m/z: Calc'd for C₇H₁₀N₂O₂[M+H]⁺155; Found, 155.

Step 3: To a stirred solution of 3-(1-methylimidazol-4-yl)propanoic acid (73 mg, 0.47 mmol, 2 eq) in DCM (5 mL) was added DCC (73 mg, 0.35 mmol, 1.5 eq), DMAP (29 mg, 0.24 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (100 mg, 0.24 mmol, 1 eq) at 0° C. under nitrogen atmosphere. The resulting mixture was stirred at room temperature for 12 h. The mixture was concentrated, and the residue was purified by reversed-phase flash chromatography with the following conditions: column, C18 silica gel; mobile phase, ACN in Water (0.5% TFA), 10% to 60% gradient in 20 min; detector, UV 254 nm. This resulted in tert-butyl (2R,3S,4S)-4-[(tert-butoxycarbonyl)oxy]-2-[(4-methoxyphenyl)methyl]-3-{[3-(1-methylimidazol-4-yl)propanoyl]oxy}pyrrolidine-1-carboxylate (140 mg, 105.9%) as a light yellow solid. MS: m/z: Calc'd for: C₂₉H₄₁N₃O₈ [M+H]⁺560; Found, 560.

Step 4: To a stirred solution of tert-butyl (2R,3S,4S)-4-[(tert-butoxycarbonyl)oxy]-2-[(4-methoxyphenyl) methyl]-3-{[3-(1-methylimidazol-4-yl)propanoyl]oxy}pyrrolidine-1-carboxylate (130 mg, 0.23 mmol, 1 eq) in DCM (5 mL) was added TFA (1 mL) dropwise at 0° C. under nitrogen atmosphere. The solution was stirred at 0° C. for 1 h. The mixture was concentrated and the crude product (80 mg) was purified by Prep-HPLC to afford (2R,3S,4S)-4-hydroxy-2-[(4-methoxyphenyl) methyl]pyrrolidin-3-yl 3-(1-methylimidazol-4-yl)propanoate; trifluoroacetic acid (28 mg, 24.4%) as a light yellow solid. MS: m/z: Calc'd for C₁₉H₂₅N₃O₄ [M+H]⁺360; Found, 360. ¹H NMR (400 MHz, Methanol-d4) δ 8.77 (s, 1H), 7.38 (s, 1H), 7.22 (d, J=8.1 Hz, 2H), 6.92 (d, J=8.1 Hz, 2H), 5.15 (d, J=3.5 Hz, 1H), 4.37 (d, J=4.2 Hz, 1H), 4.21 (s, 1H), 3.90 (s, 3H), 3.80 (s, 3H), 3.62 (dd, J=12.9, 4.3 Hz, 1H), 3.23 (d, J=12.7 Hz, 2H), 3.11-3.01 (m, 3H), 3.00-2.87 (m, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 16% B in 10 min; Wave Length: 254 nm/220 nm nm; RT1(min): 8.68

### Synthetic Procedure of V187104

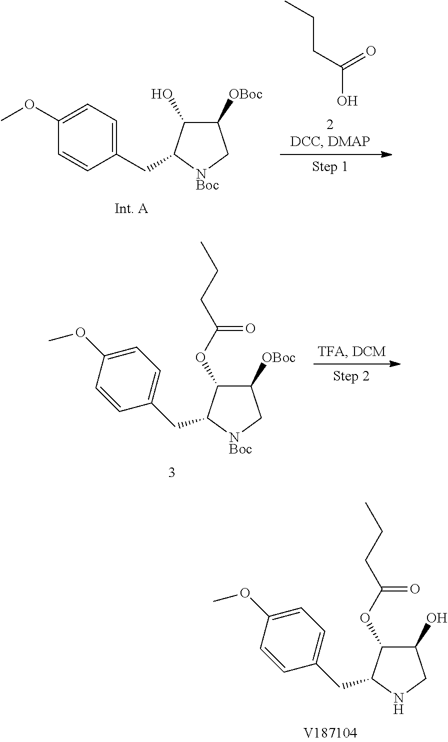

Step 1: To a stirred solution of butanoic acid (34 mg, 0.39 mmol, 2 eq) in DCM (5 mL) were added DCC (60 mg, 0.29 mmol, 1.5 eq), DMAP (24 mg, 0.20 mmol, 1 eq) and tert-butyl (2R,3S,4S)-4-[(tert-butoxycarbonyl)oxy]-3-hydroxy-2-[(4-hydroxyphenyl)methyl]pyrrolidine-1-carboxylate (80 mg, 0.20 mmol, 1 eq) at 0° C. The solution was stirred at room temperature for 12 h. The mixture was concentrated and the residue was purified by reversed-phase flash chromatography to afford tert-butyl (2R,3S,4S)-3-(butanoyloxy)-4-[(tert-butoxycarbonyl)oxy]-2-[(4-methoxyphenyl)methyl]pyrrolidine-1-carboxylate (89 mg, 92.3%) as a colorless solid. MS: m/z: Calc'd for C₂₆H₃₉NO₈ [M+H−56−56]⁺³⁸², found 382.

Step 2: To a stirred solution of tert-butyl (2R,3S,4S)-3-(butanoyloxy)-4-[(tert-butoxycarbonyl)oxy]-2-[(4-methoxyphenyl)methyl]pyrrolidine-1-carboxylate (89 mg, 0.18 mmol, 1 eq) in DCM (5 mL) was added TFA (1 mL) dropwise at 0° C. The resulting mixture was stirred at room temperature for 2 h. The mixture was concentrated and the crude product (70 mg) was purified by Prep-HPLC to afford (2R,3S,4S)-4-hydroxy-2-[(4-methoxyphenyl) methyl]pyrrolidin-3-yl butanoate; trifluoroacetic acid (17.8 mg, 24.1%) as a light yellow solid. MS: m/z: Calc'd for C₁₆H₂₃NO₄ [M+H]⁺294, found 294. ¹H NMR (400 MHz, Methanol-d4) δ 7.23 (d, J=8.2 Hz, 2H), 6.94 (d, J=8.1 Hz, 2H), 5.11 (s, 1H), 4.35 (s, 1H), 4.20 (s, 1H), 3.80 (s, 3H), 3.59 (dd, J=13.0, 4.4 Hz, 1H), 3.22 (d, J=12.7 Hz, 1H), 3.10 (dd, J=14.3, 6.7 Hz, 1H), 2.95 (dd, J=14.4, 8.9 Hz, 1H), 2.48 (t, J=7.4 Hz, 2H), 1.89-1.67 (m, 2H), 1.02 (t, J=7.4 Hz, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm nm; RT1(min): 8.68

### Synthetic Procedure of V187116

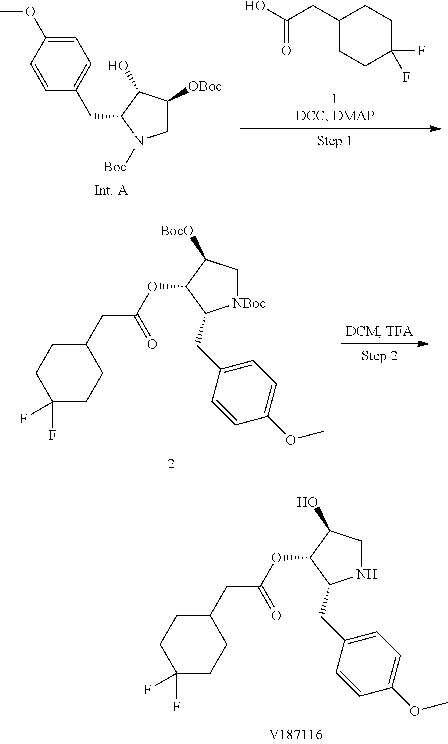

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 122.0 mg, 0.28 mmol, 1 eq.) and (2,1,3-benzoxadiazol-5-ylmethoxy)acetic acid (120 mg, 0.57 mmol, 2 eq.) in DCM (5 mL) were added DCC (178.4 mg, 0.86 mmol, 3 eq.) and DMAP (70.4 mg, 0.56 mmol, 2 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford purified by Prep-TLC to obtain tert-butyl (2R,3S,4S)-3-{[2-(2,1,3-benzoxadiazol-5-ylmethoxy)acetyl]oxy}-4-[(tert-butoxycarbonyl)oxy]-2-[(4-methoxyphenyl)methyl]pyrrolidine-1-carboxylate) as a white solid. MS: m/z: Calc'd for C₃₀H₄₃F₂NO₈ [M+H]⁺584, found 584.

Step 2: To a stirred mixture of tert-butyl (2R,3S,4S)-4-[(tert-butoxycarbonyl)oxy]-3-{[2-(4,4-difluorocyclohexyl)acetyl]oxy}-2-[(4-methoxyphenyl)methyl]pyrrolidine-1-carboxylate (65 mg, 0.11 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(4,4-difluorocyclohexyl)acetate (22.9 mg, 53.5%) as a white solid. MS: m/z: Calc'd for C₅₂H₆₂N₈O₆S [M+H]⁺383; Found, 383. ¹H NMR (400 MHz, Methanol-d₄) δ 7.17-7.09 (m, 2H), 6.89-6.81 (m, 2H), 4.81-4.79 (m, 1H), 4.11-4.09 (m, 1H), 3.77 (s, 3H), 3.55-3.53 (m, 1H), 3.33-3.22 (m, 1H), 2.79 (d, J=7.5 Hz, 2H), 2.72-2.70 (m, 1H), 2.38 (d, J=7.0 Hz, 2H), 2.12-1.98 (m, 2H), 1.94 (s, 1H), 1.85 (s, 2H), 1.84-1.70 (m, 2H), 1.39-1.37 (m, 1H), 1.34-1.32 (m, 1H). Prep-HPLC-conditions: Column: XSelect CSH Prep C18 OBD Column, 5 μm, 30*150 mm; Mobile Phase A: Water (0.05% NH₄HCO₃), Mobile Phase B:ACN; Flow rate:60 mL/min; Gradient:29 B to 59 B in 22 min; 254/220 nm; RT1:6.4; RT2:; Injection Volumn: ml; Number Of Runs:;

### Synthetic Procedure of V187123

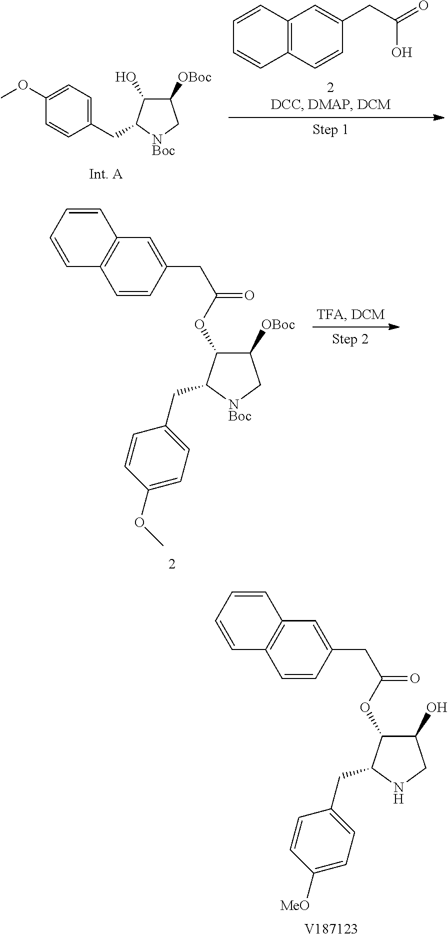

Step 1: A solution of P-naphthaleneacetic acid (88 mg, 0.47 mmol, 2 equiv) in DCM (1 mL) was treated with DCC (73.0 mg, 0.35 mmol, 1.5 equiv) and DMAP (28.8 mg, 0.23 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (100 mg, 0.23 mmol, 1 equiv) at 0° C. The resulting mixture was stirred for overnight at room temperature under nitrogen atmosphere. Desired product could be detected by LCMS. The residue was purified by Prep-TLC (PE/EA 5:1) to afford tert-butyl (2R,3S,4S)-4-[(tert-butoxycarbonyl)oxy]-2-[(4-methoxyphenyl)methyl]-3-{[2-(naphthalen-2-yl)acetyl]oxy}pyrrolidine-1-carboxylate (100 mg, 57.5%) as a yellow oil. MS: m/z: Calc'd for C₂₅H₃₄FNO₈[M+H−56−56]⁺480, found 480.

Step 2: To a stirred solution of tert-butyl (2R,3S,4S)-4-[(tert-butoxycarbonyl)oxy]-2-[(4-methoxyphenyl)methyl]-3-{[2-(naphthalen-2-yl)acetyl]oxy}pyrrolidine-1-carboxylate (119 mg, 0.20 mmol) in DCM (5 mL) was added TFA (1 mL) at 0° C. under air atmosphere. The resulting mixture was stirred for 1 h at room temperature under air atmosphere. Desired product could be detected by LCMS. The resulting mixture was concentrated under reduced pressure. The crude product (60 mg) was purified by Prep-HPLC to afford (2R,3S,4S)-4-hydroxy-2-[(4-methoxy phenyl)methyl]pyrrolidin-3-yl 2-(naphthalen-2-yl)acetate; trifluoroacetic acid (25.5 mg, 25.0%) as a white solid. MS: m/z: Calc'd for C₂₄H₂₅NO₄ [M+]+392, found 392. ¹H NMR (400 MHz, Methanol-d₄) δ 7.95-7.85 (m, 4H), 7.51-7.39 (m, 3H), 6.89-6.75 (m, 2H), 6.71-6.59 (m, 2H), 5.01 (d, J=3.6 Hz, 1H), 4.35 (d, J=4.2 Hz, 1H), 4.17-4.02 (m, 1H), 3.97 (d, J=1.8 Hz, 2H), 3.72 (d, J=2.5 Hz, 3H), 3.55-3.45 (m, 1H), 3.22 (d, J=12.6 Hz, 1H), 2.93-2.81 (m, 1H), 2.89-2.79 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 16% B to 46% B in 10 min; Wave Length: 254 nm/220 nm nm; RT1(min): 8.68

### Synthetic Procedure of V187134

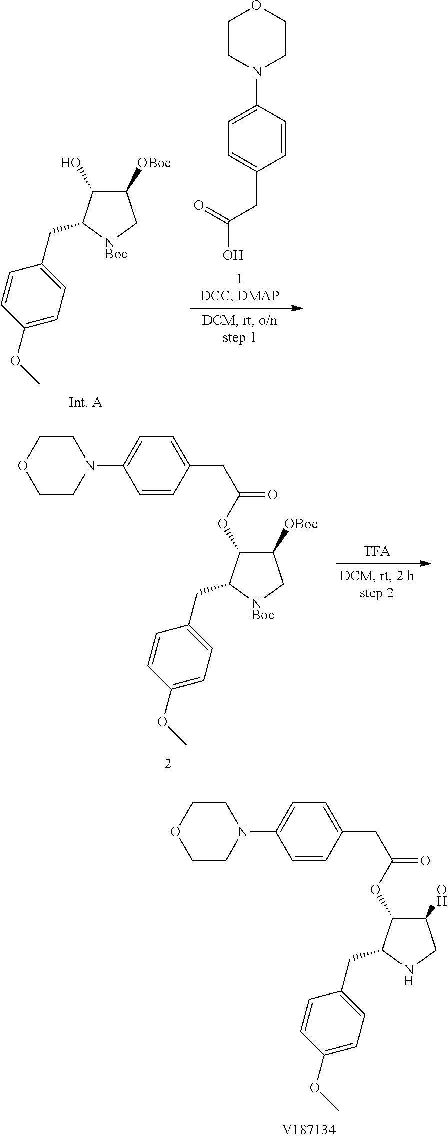

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and [4-(morpholin-4-yl)phenyl]acetic acid (104.4 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at room temperature for overnight. After completion of the reaction monitored by LCMS, the mixture was purified directly by Prep-TLC (PE/EA=5:1) to afford tert-butyl (2R,3S,4S)-4-[(tert-butoxycarbonyl)oxy]-2-[(4-methoxyphenyl)methyl]-3-({2-[4-(morpholin-4-yl)phenyl]acetyl}oxy)pyrrolidine-1-carboxylate (80 mg, 54.0%) as a white solid. MS: m/z: Calc'd for C₃₄H₄₆N₂O₉ [M+H]⁺627, found 627.

Step 2: To a stirred solution of tert-butyl (2R,3S,4S)-4-[(tert-butoxycarbonyl)oxy]-2-[(4-methoxyphenyl) methyl]-3-({2-[4-(morpholin-4-yl)phenyl]acetyl}oxy)pyrrolidine-1-carboxylate (75 mg, 0.12 mmol, 1 eq.) in DCM (3 mL) was added TFA (1 mL) dropwise at 0° C. The reaction was stirred at room temperature for 2 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-[4-(morpholin-4-yl)phenyl]acetate (27.5 mg, 53.7%) as a yellow solid. MS: m/z: Calc'd for C₂₄H₃₀N₂O₅ [M+H]⁺427, found 427. ¹H NMR (400 MHz, Methanol-d4) δ 7.32 (t, J=9.9 Hz, 2H), 7.15-7.02 (m, 2H), 6.99 (m, 2H), 6.88-6.81 (m, 2H), 5.02-4.96 (m, 1H), 4.32 (d, J=4.3 Hz, 1H), 4.16 (m, 1H), 3.86-3.84 (m, 4H), 3.80-3.73 (m, 3H), 3.72-3.65 (m, 2H), 3.61-3.52 (m, 1H), 3.22-3.19 (m, 5H), 2.91-2.81 (m, 2H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 4% B to 34% B in 10 min; Wave Length: 254 nm/220 nm nm; RT1(min): 8.68

### Synthetic Procedure of V187155

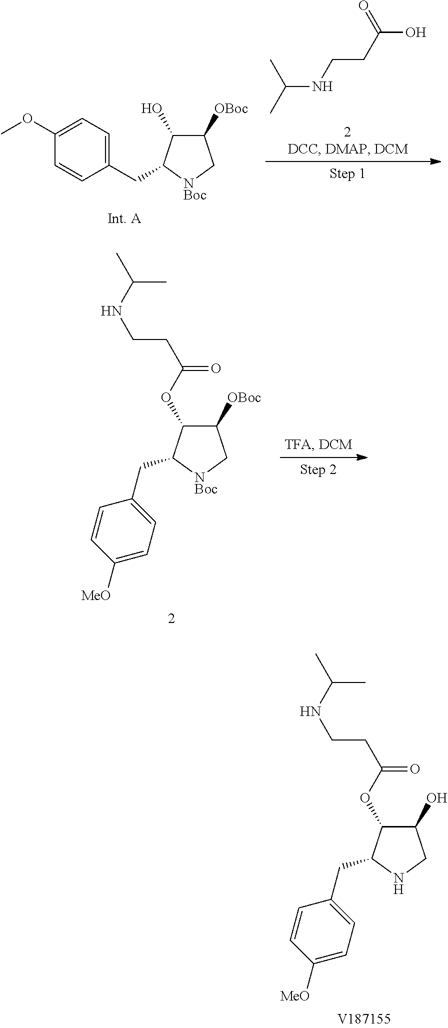

Step 1: A solution of 3-(isopropylamino)propanoic acid (50 mg, 0.37 mmol, 2 equiv) in DCM (4 mL) was treated with DCC (58.4 mg, 0.28 mmol, 1.5 equiv) and DMAP (23.0 mg, 0.18 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.18 mmol, 1 equiv) in portions at 0° C. The resulting mixture was stirred for overnight at room temperature under air atmosphere. Desired product could be detected by LCMS. The resulting mixture was filtered, the filter cake was washed with DCM (2×20 mL). The filtrate was concentrated under reduced pressure. The residue was purified by Prep-TLC (PE/EA 3:1) to afford tert-butyl (2R,3S,4S)-4-[(tert-butoxycarbonyl)oxy]-3-{[3-(isopropylamino) propanoyl]oxy}-2-[(4-methoxyphenyl)methyl]pyrrolidine-1-carboxylate (104 mg, 97.4%) as a colorless oil. MS: m/z: Calc'd for C₂₈H₄₄N₂O₈ [M+H−56−56]⁺425, found 425.

Step 2: To a stirred solution of tert-butyl (2R,3S,4S)-4-[(tert-butoxycarbonyl)oxy]-3-{[3-(isopropylamino)propanoyl]oxy}-2-[(4-methoxyphenyl)methyl]pyrrolidine-1-carboxylate (100 mg, 0.18 mmol) in DCM (5 mL) was added TFA (1 mL) at 0° C. under air atmosphere. The resulting mixture was stirred for 1 h at room temperature under air atmosphere. Desired product could be detected by LCMS. The resulting mixture was concentrated under vacuum. The crude product (60 mg) was purified by Prep-HPLC to afford (2R,3S,4S)-4-hydroxy-2-[(4-methoxyphenyl)methyl]pyrrolidin-3-yl 3-(isopropylamino)propanoate; trifluoroacetic acid (31.2 mg, 36.9%) as a white solid. MS: m/z: Calc'd for C₁₈H₂₈N₂O₄ [M+H]⁺337, found 337. ¹H NMR (400 MHz, Methanol-d₄) δ 7.25 (d, J=8.3 Hz, 2H), 6.97-6.89 (m, 2H), 5.17 (d, J=3.4 Hz, 1H), 4.44 (d, J=4.3 Hz, 1H), 4.22-4.11 (m, 1H), 3.79 (s, 3H), 3.65 (dd, J=12.7, 4.5 Hz, 1H), 3.45-3.31 (m, 1H), 3.36 (t, J=6.8 Hz, 2H), 3.22 (d, J=12.7 Hz, 1H), 3.17-2.85 (m, 4H), 1.38 (d, J=6.6 Hz, 6H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 13% B in 10 min; Wave Length: 254 nm/220 nm nm; RT1(min): 8.68

### Synthetic Procedure of V187159

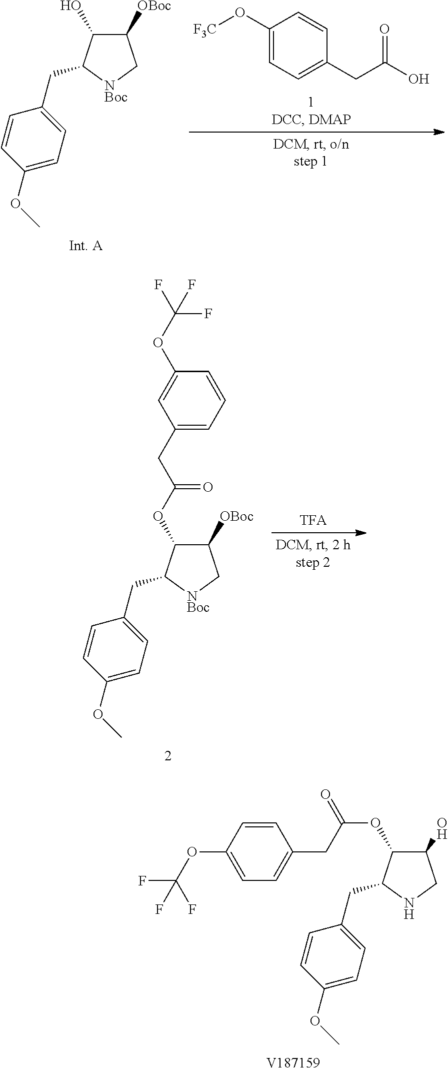

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and [4-(trifluoromethoxy)phenyl]acetic acid (103.9 mg, 0.47 mmol, 2.00 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, the resulting mixture was filtered, the filter cake was washed with DCM (1×10 mL). The filtrate was concentrated under reduced pressure. The residue was purified by Prep-TLC (CH₂Cl₂/MeOH 10:1) to afford tert-butyl (2R,3S,4S)-4-[(tert-butoxycarbonyl)oxy]-2-[(4-methoxyphenyl)methyl]-3-({2-[3-(trifluoromethoxy)phenyl]acetyl}oxy)pyrrolidine-1-carboxylate (80 mg, 54.1%) as a white solid. MS: m/z: Calc'd for C₃₁H₃₈F₃NO₉ [M−H−56−56]⁺514, found 514.

Step 2: To a stirred solution of tert-butyl (2R,3S,4S)-4-[(tert-butoxycarbonyl)oxy]-2-[(4-methoxyphenyl)methyl]-3-({2-[3-(trifluoromethoxy)phenyl]acetyl}oxy)pyrrolidine-1-carboxylate (1 eq.) in DCM (3 mL) was added TFA (1 mL) at 0° C. The reaction was stirred at r.t. for 2 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 2-[4-(trifluoromethoxy)phenyl]acetate (24.7 mg, 45.1%) as a white solid. MS: m/z: Calc'd for C₂₁H₂₂F₃NO₅ [M+H]⁺426, found 426. ¹H NMR (400 MHz, Methanol-d₄) δ 7.53-7.45 (m, 2H), 7.31 (d, J=8.3 Hz, 2H), 7.10-7.03 (m, 2H), 6.87-6.85 (m, 2H), 5.05 (s, 1H), 4.87 (s, 1H), 4.35 (d, J=4.3 Hz, 1H), 3.86 (d, J=4.1 Hz, 2H), 3.78 (d, J=1.3 Hz, 3H), 3.57-3.54 (m, 1H), 3.24 (d, J=12.6 Hz, 1H), 3.04-2.84 (m, 2H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 18% B to 48% B in 10 min; Wave Length: 254 nm/220 nm nm; RT1(min): 8.68

### Synthetic Procedure of V187160

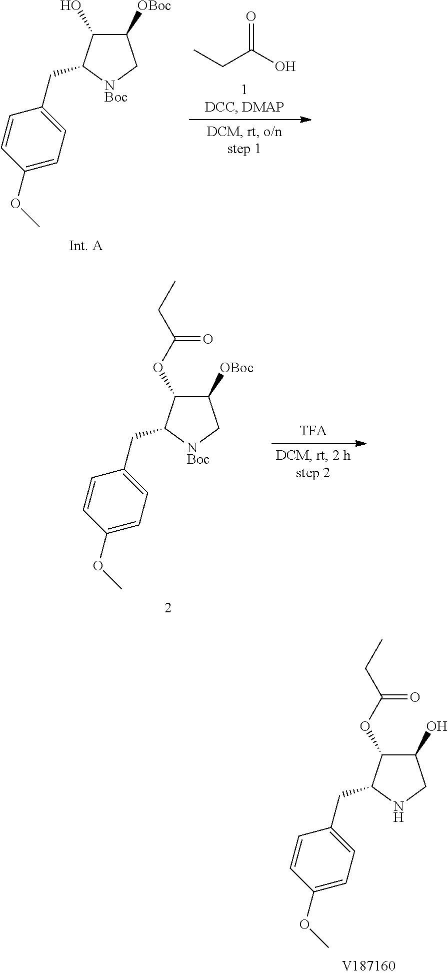

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.01 mmol, 1 eq.) and propanoic acid (34.9 mg, 0.47 mmol, 2 eq.) in DCM (3 mL) was added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS, the mixture was purified directly by Prep-TLC (PE/EA 5:1) to afford tert-butyl (2R,3S,4S)-4-[(tert-butoxycarbonyl)oxy]-2-[(4-methoxyphenyl)methyl]-3-(propanoyloxy)pyrrolidine-1-carboxylate (85 mg, 75.0%) as a white solid.

MS: m/z: Calc'd for C₂₅H₃₇NO₈[M+H−56−56]⁺368; Found, 368.

Step 2: To a stirred solution of tert-butyl (2R,3S,4S)-4-[(tert-butoxycarbonyl)oxy]-2-[(4-methoxyphenyl)methyl]-3-(propanoyloxy)pyrrolidine-1-carboxylate (80 mg, 0.16 mmol, 1 eq.) in DCM (3 mL) was added TFA (1 mL) dropwise at 0° C. The reaction was stirred at r.t. for 2 h.

The resulting mixture was concentrated under reduced pressure. The crude product (70 mg) was purified by Prep-HPLC to afford (2R,3S,4S)-4-hydroxy-2-[(4-methoxyphenyl)methyl]pyrrolidin-3-yl propanoate (12.0 mg, 25.7%) as a yellow oil. MS: m/z: Calc'd for C₁₅H₂₁NO₄[M+H]⁺280; Found, 280. 1H NMR (400 MHz, Methanol-d4) δ 7.26-7.19 (m, 2H), 6.97-6.90 (m, 2H), 5.10 (d, J=3.7 Hz, 1H), 4.36 (d, J=4.4 Hz, 1H), 4.19-4.15 (m, 1H), 3.80 (d, J=2.4 Hz, 3H), 3.61-3.55 (m, 1H), 3.23 (d, J=12.6 Hz, 1H), 3.14-3.04 (m, 1H), 2.98-2.88 (m, 1H), 2.51-2.56 (m, 2H), 1.20-1.15 (m, 3H). Prep-HPLC-conditions: Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 2% B to 7% B in 10 min; Wave Length: 254 nm/220 nm nm; RT1(min): 8.68

### Synthetic Procedure of V187162

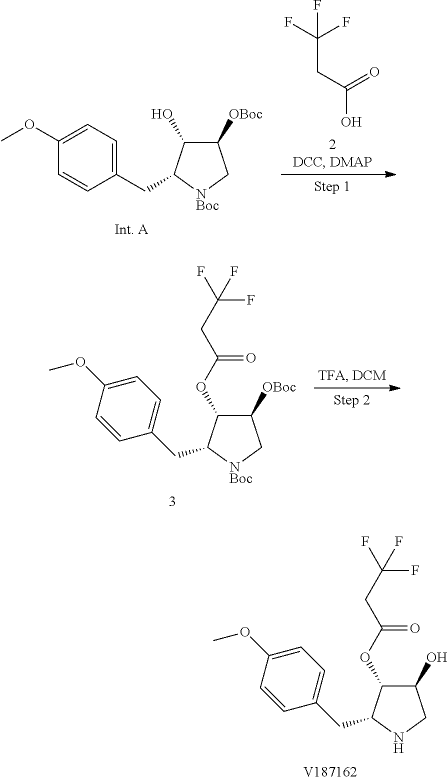

Step 1: To a stirred solution of 3,3,3-trifluoropropanoic acid (48 mg, 0.38 mmol, 2 eq) in DCM (5 mL) were added DCC (58 mg, 0.28 mmol, 1.5 eq), DMAP (23 mg, 0.19 mmol, 1 eq) and tert-butyl (2R,3S,4S)-4-[(tert-butoxycarbonyl)oxy]-3-hydroxy-2-[(4-methoxyphenyl) methyl]pyrrolidine-1-carboxylate (80 mg, 0.19 mmol, 1 eq) at 0° C. The solution was stirred at room temperature for 12 h. The mixture was concentrated and the residue was purified by reversed-phase flash chromatography to afford tert-butyl (2R,3S,4S)-4-[(tert-butoxycarbonyl)oxy]-2-[(4-methoxyphenyl)methyl]-3-[(3,3,3-trifluoropropanoyl) oxy]pyrrolidine-1-carboxylate (79 mg, 78.4%) as a colorless solid. MS: m/z: Calc'd for C₂₅H₃₄F₃NO₈[M+H−56−56]⁺422, found 422.

Step 2: To a stirred solution of tert-butyl (2R,3S,4S)-4-[(tert-butoxycarbonyl)oxy]-2-[(4-methoxyphenyl) methyl]-3-[(3,3,3-trifluoropropanoyl)oxy]pyrrolidine-1-carboxylate (79 mg, 0.15 mmol, 1 eq) in DCM (5 mL) was added TFA (1 mL) dropwise at 0° C. The resulting mixture was stirred at room temperature for 2 h. The mixture was concentrated and the crude product (60 mg) was purified by Prep-HPLC) to afford (2R,3S,4S)-4-hydroxy-2-[(4-methoxyphenyl)methyl]pyrrolidin-3-yl 3,3,3-trifluoropropanoate; trifluoroacetic acid (28.1 mg, 42.3%) as a white solid. MS: m/z: Calc'd for C₁₅H₁₈F₃NO₄ [M+H]⁺334, found 334.

¹H NMR (400 MHz, Methanol-d4) δ 7.24 (d, J=8.6 Hz, 2H), 6.98-6.91 (m, 2H), 5.22-5.16 (m, 1H), 4.40 (d, J=4.3 Hz, 1H), 4.29-4.19 (m, 1H), 3.80 (s, 3H), 3.72-3.53 (m, 3H), 3.25 (d, J=12.7 Hz, 1H), 3.11 (dd, J=14.3, 6.6 Hz, 1H), 2.96 (dd, J=14.4, 9.0 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm nm; RT1(min): 8.68

### Synthetic Procedure of V187163

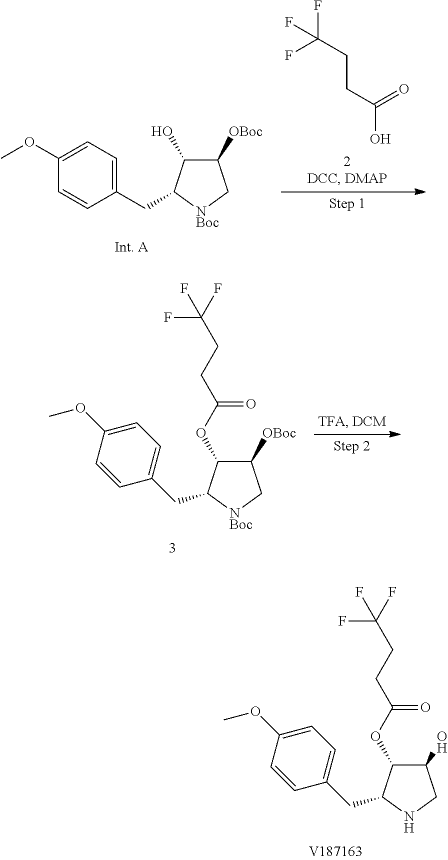

Step 1: To a stirred solution of 4,4,4-trifluorobutanoic acid (54 mg, 0.38 mmol, 2 eq) in DCM (5 mL) was added DCC (58 mg, 0.28 mmol, 1.5 eq), DMAP (23 mg, 0.19 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq) at 0° C. The solution was stirred at room temperature for 12 h. The mixture was concentrated and the residue was purified by reversed-phase flash chromatography to afford tert-butyl (2R,3S,4S)-4-[(tert-butoxycarbonyl)oxy]-2-[(4-methoxyphenyl)methyl]-3-[(4,4,4-trifluorobutanoyl) oxy]pyrrolidine-1-carboxylate (65 mg, 62.8%) as a colorless solid. MS: m/z: Calc'd for C₂₆H₃₆F₃NO₈[M+H−56−56]⁺436, found 436.

Step 2: To a stirred solution of tert-butyl (2R,3S,4S)-4-[(tert-butoxycarbonyl)oxy]-2-[(4-methoxyphenyl) methyl]-3-[(4,4,4-trifluorobutanoyl)oxy]pyrrolidine-1-carboxylate (65 mg, 0.12 mmol, 1 eq) in DCM (5 mL) was added TFA (1 mL) dropwise at 0° C. The resulting mixture was stirred at room temperature for 2 h. The mixture was concentrated and the crude product (50 mg) was purified by Prep-HPLC to afford (2R,3S,4S)-4-hydroxy-2-[(4-methoxyphenyl) methyl]pyrrolidin-3-yl 4,4,4-trifluorobutanoate; trifluoroacetic acid (27.8 mg, 50.5%) as a colorless semi-solid. MS: m/z: Calc'd for C₁₆H₂₀F₃NO₄ [M+H]⁺348, found 348. ¹H NMR (400 MHz, Methanol-d4) δ 7.24 (d, J=8.1 Hz, 2H), 6.94 (d, J=8.2 Hz, 2H), 5.15 (s, 1H), 4.39 (d, J=4.3 Hz, 1H), 4.21 (d, J=9.9 Hz, 1H), 3.80 (s, 3H), 3.61 (dd, J=12.8, 4.3 Hz, 1H), 3.23 (d, J=12.7 Hz, 1H), 3.11 (dd, J=14.3, 6.5 Hz, 1H), 2.97 (dd, J=14.4, 8.9 Hz, 1H), 2.80 (t, J=7.7 Hz, 2H), 2.69-2.51 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 7% B to 37% B in 10 min; Wave Length: 254 nm/220 nm nm; RT1(min): 8.68

### Synthetic Procedure of V187164

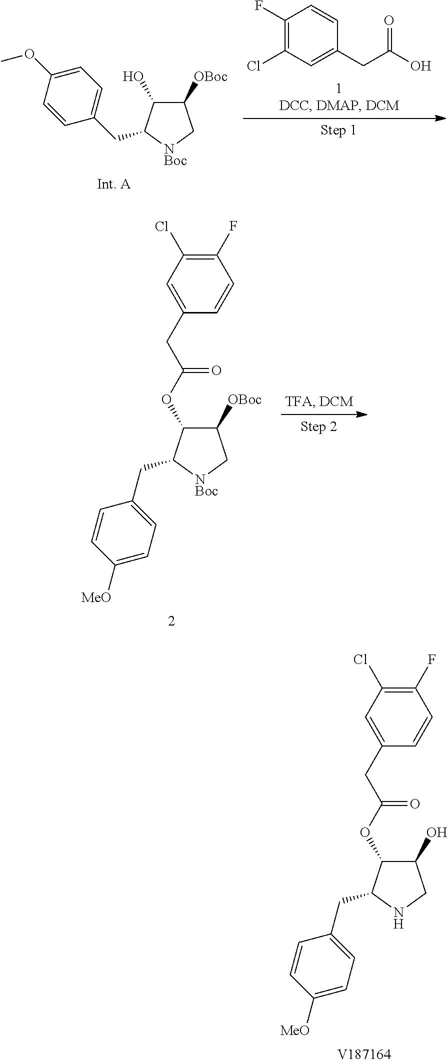

Step 1: A solution of (3-chloro-4-fluorophenyl)acetic acid (89 mg, 0.47 mmol, 2.00 equiv) in DCM (1 mL) was treated with DCC (73 mg, 0.35 mmol, 1.5 equiv) and DMAP (28.9 mg, 0.23 mmol, 1.00 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (100 mg, 0.23 mmol, 1.00 equiv) in portions at 0° C. The resulting mixture was stirred for overnight at room temperature under nitrogen atmosphere. Desired product could be detected by LCMS. The residue was purified by Prep-TLC (PE/EA 5:1) to afford tert-butyl (2R,3S,4S)-4-[(tert-butoxycarbonyl)oxy]-3-{[2-(3-chloro-4-fluorophenyl) acetyl]oxy}-2-[(4-methoxyphenyl)methyl]pyrrolidine-1-carboxylate (98 mg, 69.8%) as a yellow oil. MS: m/z: Calc'd for C₃₀H₃₇ClFNO₈[M+H−56−56]⁺482, found 482.

Step 2: To a stirred solution of tert-butyl (2R,3S,4S)-4-[(tert-butoxycarbonyl)oxy]-3-{[2-(3-chloro-4-fluorophenyl)acetyl]oxy}-2-[(4-methoxyphenyl)methyl]pyrrolidine-1-carboxylate (98 mg, 0.16 mmol) in DCM (5 mL) was added TFA (1 mL) at 0° C. under air atmosphere. The resulting mixture was stirred for 1 h at room temperature under air atmosphere. Desired product could be detected by LCMS. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl 2-(3-chloro-4-fluorophenyl)acetate; trifluoroacetic acid (24.8 mg, 29.4%) as a white semi-solid. MS: m/z: Calc'd for C₂₀H₂₁ClFNO₄ [M+H]⁺394, found 394. ¹H NMR (400 MHz, Methanol-d₄) δ 7.53 (dd, J=7.1, 2.2 Hz, 1H), 7.33-7.21 (m, 1H), 7.26-7.13 (m, 1H), 7.10-6.95 (m, 2H), 6.88-6.72 (m, 2H), 5.06 (t, J=3.7 Hz, 1H), 4.39-4.33 (m, 1H), 4.25-4.16 (m, 1H), 3.82 (d, J=5.0 Hz, 2H), 3.79 (d, J=0.9 Hz, 3H), 3.57-3.41 (m, 1H), 3.24 (d, J=12.7 Hz, 1H), 3.01 (dd, J=14.3, 7.3 Hz, 1H), 2.92 (dd, J=14.0, 8.3 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 15% B to 45% B in 10 min; Wave Length: 254 nm/220 nm nm; RT1(min): 8.68

### Synthetic Procedure of V187165

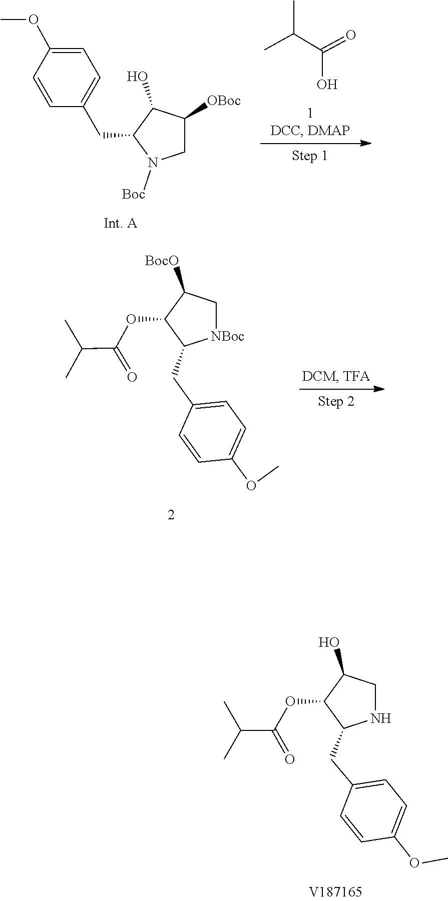

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and isobutyric acid (41.6 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was purified by Prep-TLC to obtain tert-butyl (2R,3S,4S)-4-[(tert-butoxycarbonyl)oxy]-2-[(4-methoxyphenyl)methyl]-3-[(2-methylpropanoyl)oxy]pyrrolidine-1-carboxylate (65 mg, 55.7%) as a white solid. MS: m/z: Calc'd for C₂₆H₃₉NO₈ [M+H−56−56]⁺382, found 382.

Step 2: To a stirred mixture of tert-butyl (2R,3S,4S)-4-[(tert-butoxycarbonyl)oxy]-2-[(4-methoxyphenyl)methyl]-3-[(2-methylpropanoyl)oxy]pyrrolidine-1-carboxylate (65 mg, 0.13 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-methylpropanoate (20.8 mg, 53.7%) as a white solid. MS: m/z: Calc'd for C₁₆H₂₃NO₄ [M+H]⁺294; Found, 294. 1H NMR (400 MHz, Methanol-d4) δ 7.16-7.08 (m, 2H), 6.89-6.81 (m, 2H), 4.84 (s, 1H), 4.08-4.06 (m, 1H), 3.77 (s, 3H), 3.75 (d, J=6.0 Hz, 1H), 3.54-3.52 (m, 1H), 3.36 (d, J=6.0 Hz, 2H), 2.84-2.59 (m, 2H), 1.23 (t, J=6.8 Hz, 6H). Prep-HPLC-conditions: Column: XSelect CSH Prep C18 OBD Column, 5 μm, 30*150 mm; Mobile Phase A:Water (0.05% NH₄HCO₃), Mobile Phase B:ACN; Flow rate:60 mL/min; Gradient:18 B to 48 B in 24 min; 254/220 nm; RT1:6.8; RT2:; Injection Volumn: ml; Number Of Runs:;

### Synthetic Procedure of V187166

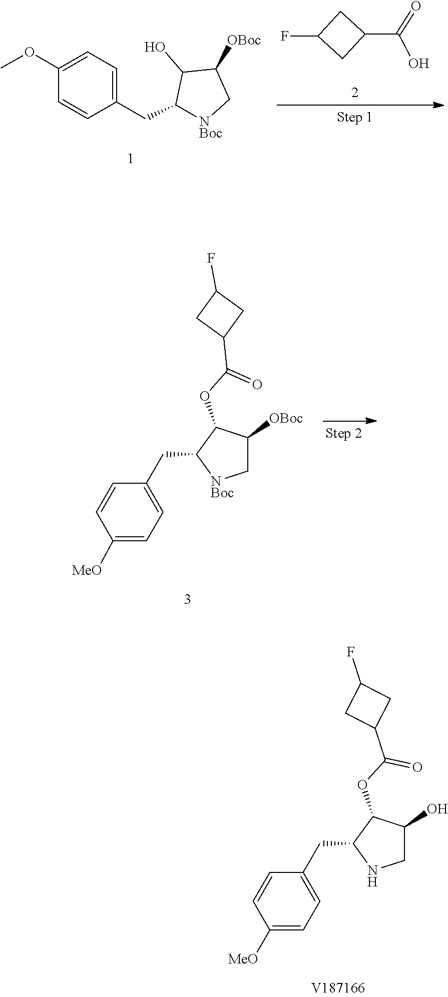

Step 1: To a stirred solution of tert-butyl (2R,4S)-4-[(tert-butoxycarbonyl)oxy]-3-hydroxy-2-[(4-methoxyphenyl)methyl]pyrrolidine-1-carboxylate (100 mg, 0.23 mmol, 1 eq.) and 3-fluorocyclobutane-1-carboxylic acid (55.8 mg, 0.47 mmol, 2 eq.) in DCM (8 mL) were added DCC (97.4 mg, 0.47 mmol, 2 eq.) and DMAP (43.3 mg, 0.35 mmol, 1.5 eq.) at 0° C. under nitrogen atmosphere. Then the resulting mixture was stirred at room temperature for overnight. Upon completion, the mixture was concentrated in vacuo. The residue was dissolved with DMSO and purified by reverse-phase column to obtain tert-butyl (2R,3S,4S)-4-[(tert-butoxycarbonyl)oxy]-3-(3-fluorocyclobutanecarbonyloxy)-2-[(4-methoxyphenyl)methyl]pyrrolidine-1-carboxylate (65 mg, 52.6%) as a white solid. MS: m/z: Calc'd for C₂₇H₃₈FNO₈ [M+H−56−56]⁺412, found 412.

Step 2: To a stirred solution of tert-butyl (2R,3S,4S)-4-[(tert-butoxycarbonyl)oxy]-3-(3-fluorocyclobutanecarbonyloxy)-2-[(4-methoxyphenyl)methyl]pyrrolidine-1-carboxylate (60 mg, 0.11 mmol, 1 eq.) in DCM (6 mL) was added TFA (1.2 mL) at room temperature for 2 h. Then the mixture was concentrated and purified by Prep-HPLC to obtain (2R,3S,4S)-4-hydroxy-2-[(4-methoxyphenyl)methyl]pyrrolidin-3-yl 3-fluorocyclobutane-1-carboxylate; trifluoroacetic acid (18.6 mg, 36.7%) as an off-white solid. MS: m/z: Calc'd for C₁₇H₂₂FNO₄ [M+H]⁺324, found 324. ¹H NMR (400 MHz, Methanol-d₄) δ 7.22 (d, J=8.1 Hz, 2H), 6.94 (d, J=8.1 Hz, 2H), 5.18-5.07 (m, 2H), 4.37 (d, J=4.4 Hz, 1H), 4.22 (dd, J=8.2, 3.3 Hz, 1H), 3.80 (s, 3H), 3.59 (dd, J=12.8, 4.3 Hz, 1H), 3.23 (d, J=12.7 Hz, 2H), 3.10 (dd, J=14.5, 6.6 Hz, 1H), 2.96 (dd, J=14.3, 8.9 Hz, 1H), 2.74-2.38 (m, 4H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 4% B to 34% B in 10 min; Wave Length: 254 nm/220 nm nm; RT1(min): 8.68

### Synthetic Procedure of V187168

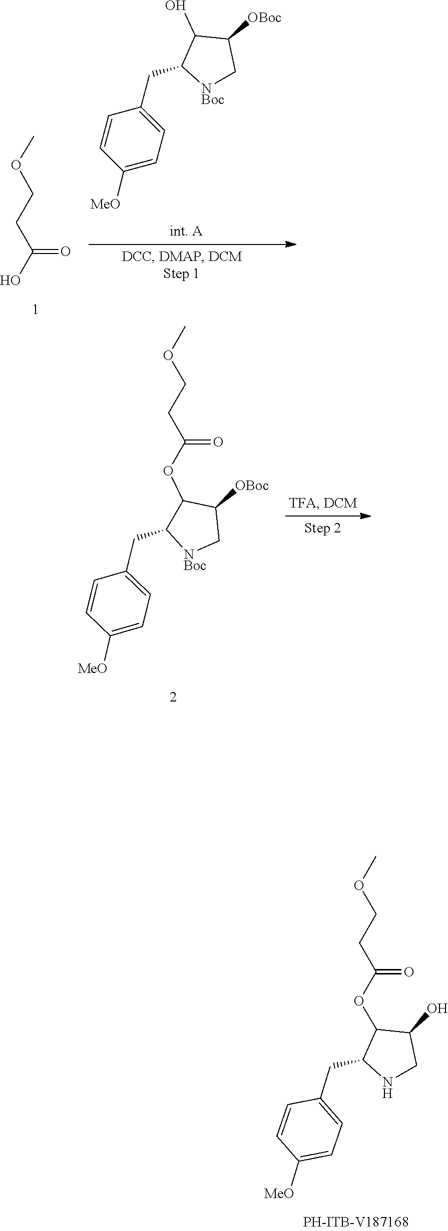

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-methoxypropanoic acid (49.1 mg, 0.47 mmol, 2 equiv) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was purified by Prep-TLC (PE/EA=2/1 as eluent) to afford tert-butyl (2R,4S)-4-[(tert-butoxycarbonyl)oxy]-2-[(4-methoxyphenyl)methyl]-3-{[3-(1,3-thiazol-4-yl)propanoyl]oxy}pyrrolidine-1-carboxylate (120 mg, 94.0%) as a yellow oil. MS: m/z: Calc'd for C₂₆H₃₉NO₉ [M+Na]⁺532, found 532.

Step 2: To a stirred mixture of tert-butyl (2R,4S)-4-[(tert-butoxycarbonyl)oxy]-2-[(4-methoxyphenyl)methyl]-3-[(3-methoxypropanoyl)oxy]pyrrolidine-1-carboxylate (100 mg, 0.19 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford ((2R,4S)-4-hydroxy-2-[(4-methoxyphenyl)methyl]pyrrolidin-3-yl 3-methoxypropanoate(37.3 mg, 44.90%) as a white semi-solid. MS: m/z: Calc'd for C₁₆H₂₃NO₅[M+H]⁺310, found 310. ¹H NMR (400 MHz, Methanol-d₄) δ 7.24 (d, J=8.3 Hz, 2H), 6.92 (d, J=8.2 Hz, 2H), 5.11 (d, J=3.3 Hz, 1H), 4.36 (d, J=3.4 Hz, 1H), 4.17-4.15 (m, 1H), 3.79 (s, 3H), 3.73 (t, J=5.9 Hz, 2H), 3.60-3.58 (m, 1H), 3.39 (s, 3H), 3.23-3.21 (m, 1H), 3.09-3.07 (m, 1H), 2.98-2.96 (m, 1H), 2.73-2.71 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 3% B to 33% B in 10 min; Wave Length: 254 nm/220 nm nm; RT1(min): 8.5

### Synthetic Procedure of V187169

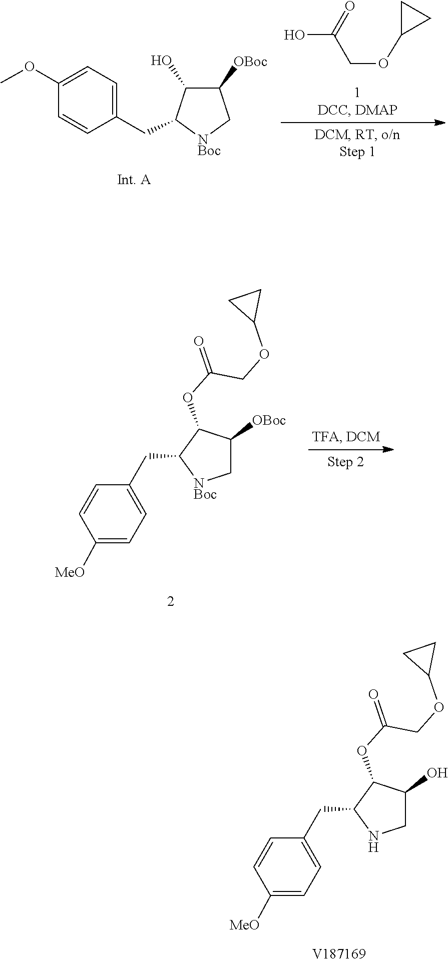

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and cyclopropoxyacetic acid (21.9 mg, 0.19 mmol, 1.0 eq.) in DCM (5 mL) were added DCC (58.5 mg, 0.28 mmol, 1.5 eq.) and DMAP (23.1 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-[(2-cyclopropoxyacetyl)oxy]-2-[(4-methoxyphenyl)methyl]pyrrolidine-1-carboxylate (60 mg, 60.89%) as a light yellow oil. MS: m/z: Calc'd for C₂₇H₃₉NO₉ [M+H−56−56]⁺310, found 310.

Step 2: To a stirred mixture of tert-butyl (2R,3S,4S)-4-[(tert-butoxycarbonyl)oxy]-3-[(2-cyclopropoxyacetyl)oxy]-2-[(4-methoxyphenyl)methyl]pyrrolidine-1-carboxylate (55 mg, 0.11 mmol, 1.0 eq.) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-cyclopropoxyacetate; trifluoroacetic acid (13.5 mg, 29.2%) as a white solid. MS: m/z: Calc'd for C₁₇H₂₃NO₅ [M+H]⁺322, found 322. ¹H NMR (400 MHz, Methanol-d₄) δ 7.28-7.20 (m, 2H), 6.94 (dd, J=7.6, 1.6 Hz, 2H), 5.20-5.15 (m, 1H), 4.41 (d, J=4.3 Hz, 1H), 4.32 (d, J=4.5 Hz, 2H), 4.27-4.19 (m, 1H), 3.80 (s, 3H), 3.62-3.58 (m, 2H), 3.24 (d, J=12.7 Hz, 1H), 3.10 (dd, J=14.2, 6.8 Hz, 1H), 3.01-2.95 (m, 1H), 0.72-0.63 (m, 2H), 0.63-0.49 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.68

### Synthetic Procedure of V187170

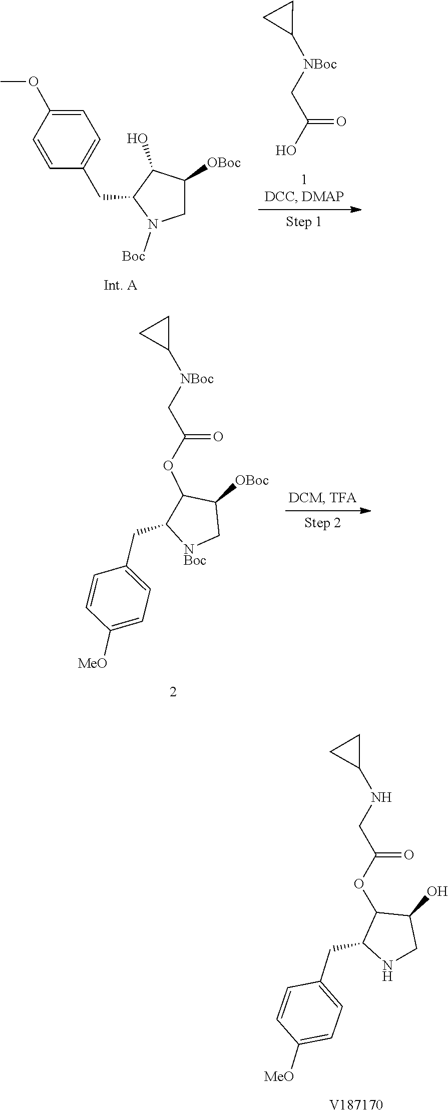

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int. A, 100 mg, 0.23 mmol, 1 eq.) and[(tert-butoxycarbonyl)(cyclopropyl)amino]acetic acid (101.6 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-3-({2-[(tert-butoxycarbonyl)(cyclopropyl)amino]acetyl}oxy)-4-[(tert-butoxycarbonyl)oxy]-2-[(4-methoxyphenyl)methyl]pyrrolidine-1-carboxylate (70 mg, 47.7%) as a white solid. MS: m/z: Calc'd for C₃₂H₄₈N₂O₁₀ [M+Na]⁺643, found 643.

Step 2: To a stirred mixture of tert-butyl (2R,3S,4S)-3-({2-[(tert-butoxycarbonyl) (cyclopropyl) amino]acetyl}oxy)-4-[(tert-butoxycarbonyl)oxy]-2-[(4-methoxyphenyl) methyl]pyrrolidine-1-carboxylate (65 mg, 0.10 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(cyclopropylamino)acetate (19.3 mg, 57.0%) as a white solid. MS: m/z: Calc'd for C₁₇H₂₄N₂O₄ [M+H]⁺421; Found, 421. ¹H NMR (400 MHz, Methanol-d₄) δ 7.21-7.10 (m, 2H), 6.94-6.81 (m, 2H), 4.24-4.13 (m, 1H), 3.77 (s, 3H), 3.63-3.51 (m, 1H), 3.46-3.33 (m, 2H), 2.82-2.80 (m, 2H), 2.79-2.77 (m, 2H), 2.75-2.73 (m, 1H), 2.34-2.24 (m, 1H), 0.59-0.44 (m, 2H), 0.41-0.35 (m, 2H). Prep-HPLC-conditions: Column: XSelect CSH Prep C18 OBD Column, 5 μm, 30*150 mm; Mobile Phase A:Water (0.05% NH₄HCO₃), Mobile Phase B:ACN; Flow rate:60 mL/min; Gradient:6 B to 36 B in 30 min; 254/220 nm; RT1:8.18; RT2:; Injection Volumn: ml; Number Of Runs:;

### Synthetic Procedure of V187171

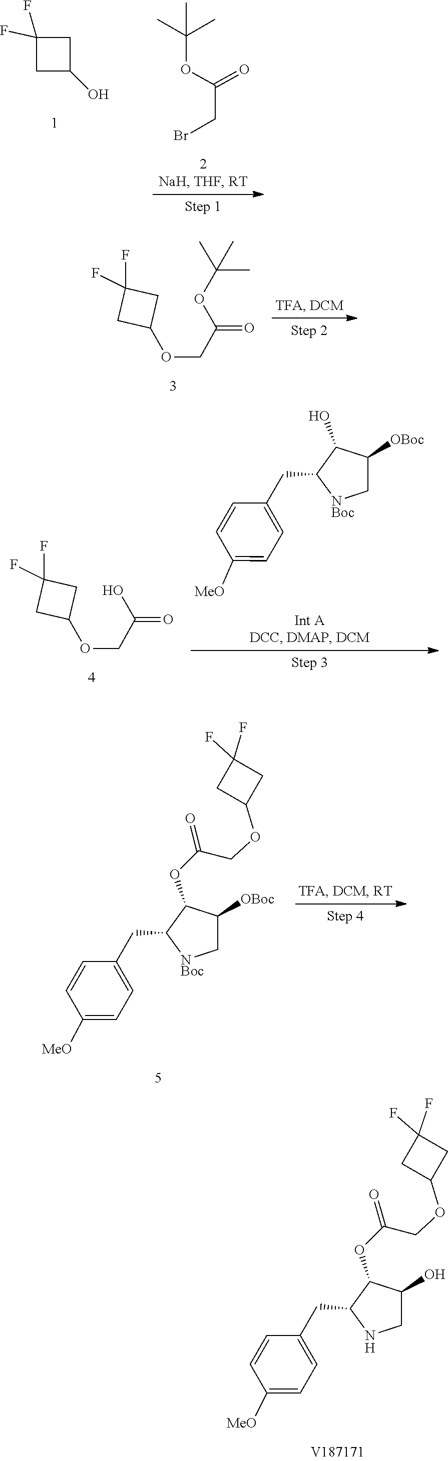

Step 1: A solution of 3,3-difluorocyclobutan-1-ol (500 mg, 4.62 mmol, 1 equiv) in THF (5 mL) was treated with NaH (333 mg, 13.87 mmol, 3 equiv) at 0° C. under nitrogen atmosphere, and the mixture was stirred at the same temperature for 1 h, followed by the addition of tert-butyl 2-bromoacetate (2707 mg, 13.87 mmol, 3 equiv). The resulting mixture was stirred for 3 h at room temperature under nitrogen atmosphere. The mixture was concentrated under vacuum. The residue was purified by Prep-TLC (PE/EA 5:1) to afford tert-butyl 2-(3,3-difluorocyclobutoxy)acetate (300 mg, 29.1%) as a yellow oil.

Step 2: To a stirred mixture of tert-butyl 2-(3,3-difluorocyclobutoxy)acetate (500 mg, 2.25 mmol, 1 equiv) in DCM (5 mL) was added TFA (1 mL, 13.46 mmol, 5.9 equiv) at 0° C. and the resulting mixture was stirred at room temperature for 1 h. The reaction mixture was concentrated under vacuum to give (3,3-difluorocyclobutoxy)acetic acid (300 mg, 80.2%) as a yellow oil. MS: m/z: Calc'd for C₆H₈F₂O₃[M−H]⁺165, found 165.

Step 3: A solution of (3,3-difluorocyclobutoxy)acetic acid (78 mg, 0.47 mmol, 2 equiv) in DCM (5 mL) were treated with DCC (73 mg, 0.35 mmol, 1.5 equiv) and DMAP (29 mg, 0.23 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (100 mg, 0.24 mmol, 1.00 equiv). The resulting mixture was stirred at room temperature for overnight under air atmosphere. The resulting mixture was concentrated under vacuum. This resulted in tert-butyl (2R,3S,4S)-4-[(tert-butoxycarbonyl)oxy]-3-{[2-(3,3-difluorocyclobutoxy)acetyl]oxy}-2-[(4-methoxyphenyl)methyl]pyrrolidine-1-carboxylate (36 mg, 26.6%) as a yellow oil. MS: m/z: Calc'd for C₂₈H₃₉F₂NO₉ [M−56−56]⁺460, found 460.

Step 4: To a stirred mixture of tert-butyl (2R,3S,4S)-4-[(tert-butoxycarbonyl)oxy]-3-{[2-(3,3-difluorocyclobutoxy)acetyl]oxy}-2-[(4-methoxyphenyl)methyl]pyrrolidine-1-carboxylate (36 mg, 0.06 mmol, 1 equiv) in DCM (5 mL) was added TFA (1 mL) at 0° C. The resulting mixture was stirred for 1 h at room temperature. The mixture was concentrated under reduced pressure. The crude product (30 mg) was purified by Prep-HPLC to afford (2R,3S,4S)-4-hydroxy-2-[(4-methoxyphenyl)methyl]pyrrolidin-3-yl 2-(3,3-difluorocyclobutoxy)acetate; trifluoroacetic acid (18.6 mg, 60.4%) as a white solid. MS: m/z: Calc'd for C₂₈H₃₉F₂NO₉ [M+H]⁺372, found 372. ¹H NMR (400 MHz, Methanol-d₄) δ 7.28-7.20 (m, 2H), 6.98-6.89 (m, 2H), 5.16 (d, J=3.4 Hz, 1H), 4.43-4.37 (m, 1H), 4.34-4.11 (m, 4H), 3.79 (s, 3H), 3.60 (dd, J=12.7, 4.4 Hz, 1H), 3.23 (d, J=12.7 Hz, 1H), 3.09 (dd, J=14.3, 6.9 Hz, 1H), 3.03-2.82 (m, 3H), 2.71-2.54 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 7% B to 37% B in 10 min; Wave Length: 254 nm/220 nm nm; RT1(min): 8.68

### Synthetic Procedure of V187173

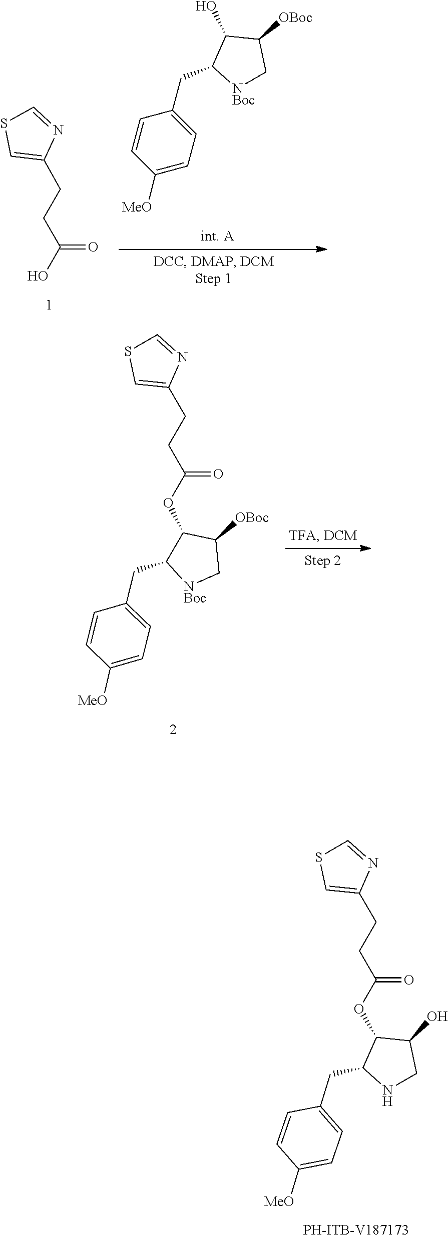

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and 3-(1,3-thiazol-4-yl)propanoic acid (59.3 mg, 0.37 mmol, 2 eq.) in DCM (5 mL) were added DCC (116.9 mg, 0.56 mmol, 3 eq.) and DMAP (34.6 mg, 0.28 mmol, 1.5 eq.) at 0° C. under nitrogen atmosphere. The resulting reaction mixture was stirred at ambient temperature for overnight. Upon completion, the reaction mixture was concentrated in vacuo. The resulting residue was purified by Prep-TLC (PE/EA=2/1 as eluent) to afford tert-butyl (2R,4S)-4-[(tert-butoxycarbonyl)oxy]-2-[(4-methoxyphenyl)methyl]-3-{[3-(1,3-thiazol-4-yl)propanoyl]oxy}pyrrolidine-1-carboxylate (100 mg, 94.0%) as a yellow oil. MS: m/z: Calc'd for C₂₈H₃₈N₂O₈S [M+H]⁺563, found 563.

Step 2: To a stirred mixture of tert-butyl (2R,4S)-4-[(tert-butoxycarbonyl)oxy]-2-[(4-methoxyphenyl) methyl]-3-{[3-(1,3-thiazol-4-yl)propanoyl]oxy}pyrrolidine-1-carboxylate (100 mg, 0.17 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 3-(1,3-thiazol-4-yl)propanoate (43.8 mg, 51.5%) as a white solid. MS: m/z: Calc'd for C₁₈H₂₂N₂O₄S [M+H]⁺363, found 363. ¹H NMR (400 MHz, DMSO-d₆) δ 8.99-8.97 (m, 1H), 7.41-7.28 (m, 1H), 7.24-7.10 (m, 2H), 6.98-6.84 (m, 2H), 5.09 (s, 1H), 4.31 (d, J=4.3 Hz, 1H), 4.16-4.14 (m, 1H), 3.79 (s, 3H), 3.63-3.47 (m, 1H), 3.21-3.19 (m, 3H), 3.08-2.81-2.79 (m, 4H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm nm; RT1(min): 8.68

### Synthetic Procedure of V187175

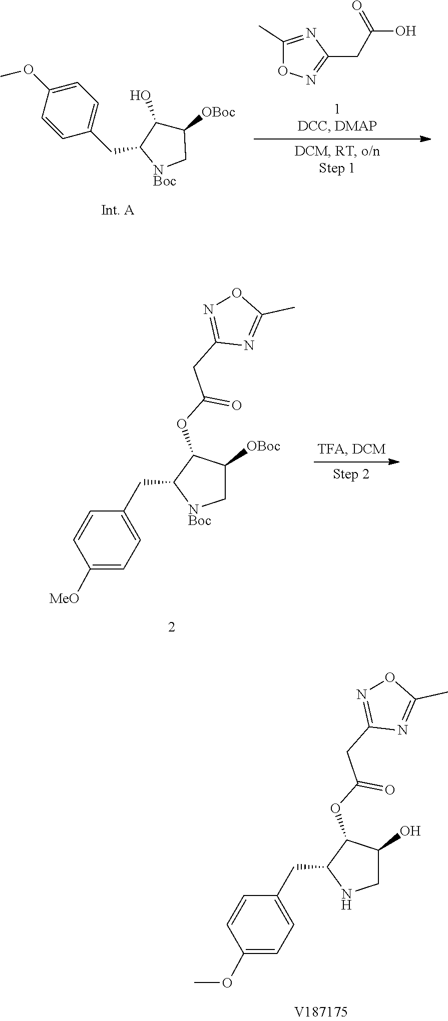

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.19 mmol, 1.0 eq.) and (5-methyl-1,2,4-oxadiazol-3-yl)acetic acid (53.7 mg, 0.38 mmol, 2.0 eq.) in DCM (5 mL) were added DCC (58.5 mg, 0.28 mmol, 1.5 eq.) and DMAP (23.1 mg, 0.19 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-2-[(4-methoxyphenyl)methyl]-3-{[2-(5-methyl-1,2,4-oxadiazol-3-yl)acetyl]oxy}pyrrolidine-1-carboxylate (65 mg, 62.8%) as a light yellow oil. MS: m/z: Calc'd for C₂₇H₃₇N₃O₉ [M+H−56−56]⁺436, found 436.

Step 2: To a stirred mixture of tert-butyl (2R,3S,4S)-4-[(tert-butoxycarbonyl)oxy]-2-[(4-methoxyphenyl) methyl]-3-{[2-(5-methyl-1,2,4-oxadiazol-3-yl)acetyl]oxy}pyrrolidine-1-carboxylate (60 mg, 0.11 mmol) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(5-methyl-1,2,4-oxadiazol-3-yl)acetate; trifluoroacetic acid (19.7 mg, 38.07%) as a white solid. MS: m/z: Calc'd for C₁₇H₂₁N₃O₅ [M+H]⁺348, found 348. ¹H NMR (400 MHz, Methanol-d₄) δ 7.28-7.20 (m, 2H), 6.98-6.89 (m, 2H), 5.13 (d, J=3.4 Hz, 1H), 4.42 (d, J=4.3 Hz, 1H), 4.23-4.19 (m, 1H), 4.04-3.97 (m, 1H), 3.80 (s, 3H), 3.59 (dd, J=12.7, 4.4 Hz, 1H), 3.24 (d, J=12.7 Hz, 1H), 3.08 (dd, J=14.2, 7.0 Hz, 1H), 2.96 (dd, J=14.3, 8.6 Hz, 1H), 2.63 (s, 3H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm nm; RT1(min): 8.68

### Synthetic Procedure of V187176

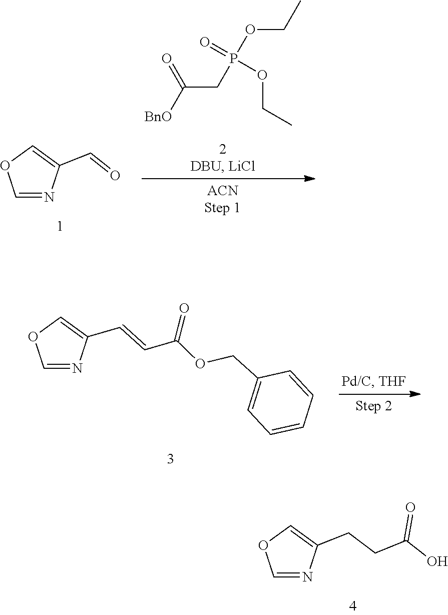 [SMILES: CCOP(=O)(CC(=O)OCc1ccccc1)OCC ; O=Cc1cocn1 ; O=C(/C=C/c1cocn1)OCc1ccccc1 ; O=C(O)CCc1cocn1]

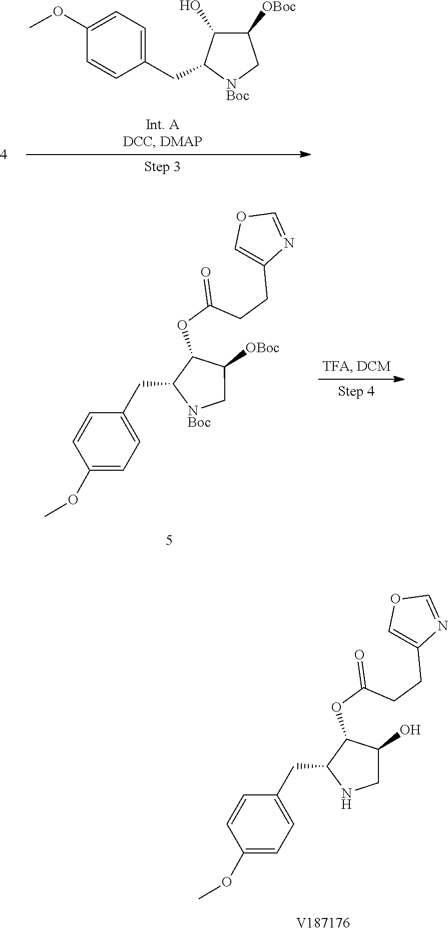

Step 1: A solution of benzyl 2-(diethoxyphosphoryl)acetate (1.18 g, 4.12 mmol, 2 eq) in ACN (10 mL) was treated with DBU (376 mg, 2.47 mmol, 1.2 eq) and LiCl (122 mg, 2.88 mmol, 1.4 eq) for 30 min at 0° C. under nitrogen atmosphere followed by the addition of 1,3-oxazole-4-carbaldehyde (200 mg, 2.06 mmol, 1 eq). The resulting mixture was stirred at room temperature for 12 h. The mixture was concentrated and the residue was applied onto a silica gel column eluting with ethyl acetate/petroleum ether (1:10). This resulted in benzyl (2E)-3-(1,3-oxazol-4-yl)prop-2-enoate (340 mg, 71.9%) as a colorless oil. MS: m/z: Calc'd for C₁₃H₁₁NO₃ [M+H]⁺230; Found, 230.

Step 2: To a stirred solution of benzyl (2E)-3-(1,3-oxazol-4-yl)prop-2-enoate (320 mg, 1.40 mmol, 1 eq) in THF (15 mL) was added Pd/C (97 mg, 0.91 mmol, 0.7 eq) at room temperature under nitrogen atmosphere. The flask was evacuated and flushed three times with nitrogen, followed by flushing with hydrogen. The mixture was stirred 2 h at room temperature under an atmosphere of hydrogen (pressure tank reactor 10 atm). The resulting mixture was filtered, the filter cake was washed with MeOH (3×30 mL). The filtrate was concentrated under reduced pressure. This resulted in 3-(1,3-oxazol-4-yl)propanoic acid (130 mg, 66.0%) as a white solid. MS: m/z: Calc'd for C₆H₇NO₃ [M+H]⁺142; Found, 142.

Step 3: To a stirred solution of 3-(1,3-oxazol-4-yl)propanoic acid (53 mg, 0.38 mmol, 2 eq) in DCM (5 mL) was added DCC (58 mg, 0.28 mmol, 1.5 eq), DMAP (23 mg, 0.19 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.19 mmol, 1 eq) at 0° C. under nitrogen atmosphere. The resulting mixture was stirred at room temperature for 12 h. The mixture was concentrated and the residue was purified by reversed-phase flash chromatography with the following conditions: column, C18 silica gel; mobile phase, ACN in Water (0.5% TFA), 10% to 100% gradient in 30 min; detector, UV 254 nm. This resulted in tert-butyl (2R,3S,4S)-4-[(tert-butoxycarbonyl)oxy]-2-[(4-methoxyphenyl)methyl]-3-{[3-(1,3-oxazol-4-yl)propanoyl]oxy}pyrrolidine-1-carboxylate (95 mg, 92.00%) as a light yellow solid. MS: m/z: Calc'd for: C₂₈H₃₈N₂O₉ [M+H−56−56]⁺435; Found, 435.

Step 4: To a stirred solution of tert-butyl (2R,3S,4S)-4-[(tert-butoxycarbonyl)oxy]-2-[(4-methoxyphenyl) methyl]-3-{[3-(1,3-oxazol-4-yl)propanoyl]oxy}pyrrolidine-1-carboxylate (90 mg, 0.17 mmol, 1 eq) in DCM (5 mL) was added TFA (1 mL) dropwise at 0° C. under nitrogen atmosphere. The resulting mixture was stirred at room temperature for 2 h. The mixture was concentrated and the crude product (70 mg) was purified by Prep-HPLC to afford (2R,3S,4S)-4-hydroxy-2-[(4-methoxyphenyl) methyl]pyrrolidin-3-yl 3-(1,3-oxazol-4-yl)propanoate; trifluoroacetic acid (16.6 mg, 21.9%) as a light yellow solid. MS: m/z: Calc'd for C₁₈H₂₂N₂O₅ [M+H]⁺347; Found, 347. ¹H NMR (400 MHz, Methanol-d4) δ 8.18 (s, 1H), 7.76 (s, 1H), 7.19 (d, J=8.2 Hz, 2H), 6.92 (d, J=8.2 Hz, 2H), 5.10 (d, J=3.3 Hz, 1H), 4.36 (d, J=4.3 Hz, 1H), 4.23-4.14 (m, 1H), 3.80 (s, 3H), 3.59 (dd, J=12.8, 4.5 Hz, 1H), 3.22 (d, J=12.7 Hz, 1H), 3.05 (dd, J=14.3, 6.9 Hz, 1H), 2.98-2.89 (m, 3H), 2.85 (dd, J=9.7, 6.7 Hz, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm nm; RT1(min): 8.68

### Synthetic Procedure of V187177

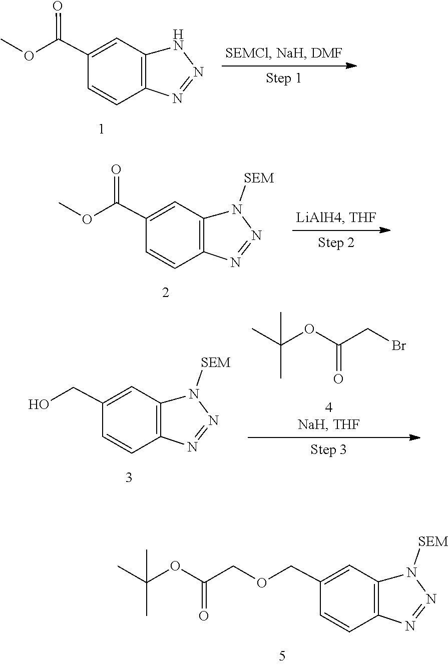

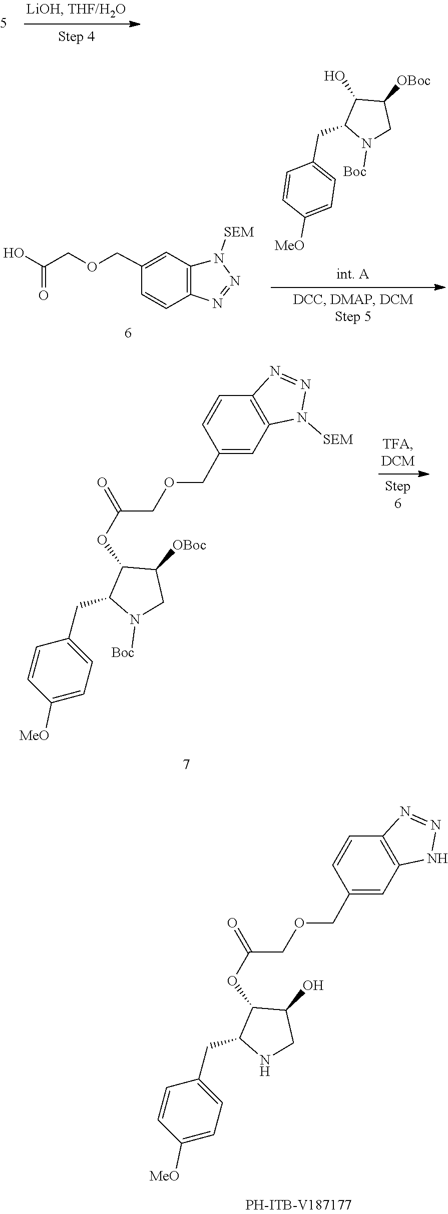

Step 1: To a stirred solution of methyl 3H−1,2,3-benzotriazole-5-carboxylate (200 mg, 1.12 mmol, 1 equiv) in DMF (10 mL) was added NaH (54.18 mg, 2.25 mmol, 2 equiv) in portions at 0° C. The mixture was stirred for 0.5 hours and then add SEM-Cl (376.42 mg, 2.25 mmol, 2 equiv). The resulting mixture was stirred overnight at RT. Upon completion, the reaction mixture was concentrated in vacuo. The residue was purified by Prep-TLC (PE/EA, 2/1) to afford methyl 3-{[2-(trimethylsilyl)ethoxy]methyl}-1,2,3-benzotriazole-5-carboxylate (180 mg, 51.8%) as a white oil.

MS: m/z: Calc'd for C₁₄H₂₁N₃O₃Si[M+H]⁺308, found 308.

Step 2: To a stirred solution of methyl 3-{[2-(trimethylsilyl)ethoxy]methyl}-1,2,3-benzotriazole-5-carboxylate (180 mg, 0.58 mmol, 1 equiv) in THF (20 mL) was added LiAlH₄ (44.4 mg, 1.17 mmol, 2 equiv) at 0° C. The resulting mixture was stirred overnight at RT. The reaction was quenched by the addition of Na₂SO₄-10H₂O solution (200 ml) at 0° C. The resulting mixture was concentrated under reduced pressure to afford (3-{[2-(trimethylsilyl)ethoxy]methyl}-1,2,3-benzotriazol-5-yl)methanol (160 mg, 97.8%) as yellow oil. MS: m/z: Calc'd for C₁₃H₂₁N₃O₂Si[M+H]⁺280, found 280.

Step 3: To a stirred solution of (3-{[2-(trimethylsilyl)ethoxy]methyl}-1,2,3-benzotriazol-5-yl)methanol (175 mg, 0.62 mmol, 1 equiv) in THF (15 mL) was added NaH (45.09 mg, 1.87 mmol, 3 equiv) in portions at 0° C. The mixture was stirred for 0.5 hours and then add tert-butyl 2-bromoacetate (366.5 mg, 1.87 mmol, 3 equiv). The resulting mixture was stirred overnight at RT. Upon completion, the reaction mixture was concentrated in vacuo. The residue was purified by reversed phase flash with the following conditions (0.05% TFA in water/ACN) to afford tert-butyl 2-[(3-{[2-(trimethylsilyl)ethoxy]methyl}-1,2,3-benzotriazol-5-yl)methoxy]acetate (150 mg, 60.8%) as a white oil. MS: m/z: Calc'd for C₁₉H₃₁N₃O₄Si[M+H]⁺394, found 394.

Step 4: To a stirred solution of tert-butyl 2-[(3-{[2-(trimethylsilyl)ethoxy]methyl}-1,2,3-benzotriazol-5-yl)methoxy]acetate (125 mg, 0.31 mmol, 1 equiv) in THF (10 mL) and H₂O (5 mL) was added LiOH (22.82 mg, 0.95 mmol, 3 equiv) at RT. The resulting mixture was stirred for overnight. Upon completion, the mixture was neutralized to pH=7 with 1M HCl. and extracted with ethyl acetate. The combined organic layers were washed with brine and dried over anhydrous sodium sulfate. After filtration, the filtrate was concentrated under reduced pressure. The residue was purified by reversed phase flash with the following conditions (0.05% TFA/ACN) to afford [(3-{[2-(trimethylsilyl)ethoxy]methyl}-1,2,3-benzotriazol-5-yl)methoxy]acetic acid (100 mg, 93.3%) as a white oil. MS: m/z: Calc'd for C₁₅H₂₃N₃O₄Si[M+H]⁺338, found 338.

Step 5: To a stirred solution of [(3-{[2-(trimethylsilyl)ethoxy]methyl}-1,2,3-benzotriazol-5-yl)methoxy]acetic acid (95 mg, 0.28 mmol, 1 equiv) and DCC (174.26 mg, 0.84 mmol, 3 equiv), DMAP (51.59 mg, 0.42 mmol, 1.5 equiv) in DCM (10 mL) was added tert-butyl (2R,3S,4S)-4-[(tert-butoxycarbonyl)oxy]-3-hydroxy-2-[(4-methoxyphenyl)methyl]pyrrolidine-1-carboxylate (119.23 mg, 0.282 mmol, 1 equiv) at RT. The resulting mixture was stirred for overnight. The reaction mixture was concentrated in vacuo. The residue was purified by reversed phase flash with the following conditions (0.05% TFA in water/ACN) to afford tert-butyl (2R,3S,4S)-4-[(tert-butoxycarbonyl)oxy]-2-[(4-methoxyphenyl)methyl]-3-({2-[(3-{[2-(trimethylsilyl) ethoxy]methyl}-1,2,3-benzotriazol-5-yl)methoxy]acetyl}oxy)pyrrolidine-1-carboxylate (120 mg, 57.3%) as a white solid. MS: m/z: Calc'd for C₃₇H₅₄N₄O₁₀Si[M+H]⁺743, found 743.

Step 6: To a stirred solution of tert-butyl (2R,3S,4S)-4-[(tert-butoxycarbonyl)oxy]-2-[(4-methoxyphenyl)methyl]-3-({2-[(3-{[2-(trimethylsilyl)ethoxy]methyl}-1,2,3-benzotriazol-5-yl)methoxy]acetyl}oxy)pyrrolidine-1-carboxylate (90 mg, 0.12 mmol, 1 equiv) in DCM (9 mL) was added TFA (3 mL). The resulting mixture was stirred for overnight. Upon completion, the reaction mixture was concentrated in vacuo. The residue was purified by Prep-HPLC to afford MS: m/z: Calc'd for C₂₁H₂₄N₄O₅ [M+H]⁺413, found 413. ¹H NMR (400 MHz, Methanol-d₄) δ 7.94 (s, 1H), 7.89 (d, J=8.6 Hz, 1H), 7.57 (d, J=8.6, 1H), 7.24-7.16 (m, 2H), 6.98-6.85 (m, 2H), 5.23-5.14 (m, 1H), 4.86 (s, 2H), 4.40 (d, J=6.9, 3H), 4.23 (m, 1H), 3.79 (s, 3H), 3.58-3.56 (m, 1H), 3.22 (d, J=12.7 Hz, 1H), 3.09-3.07 (m, 1H), 2.94-2.92 (m, 1H). Prep-HPLC conditions: UV 254 nm/220 nm Xselect CSH Prep C18 Column, 30*150 mm, 5 μm Water (0.05% TFA) ACN 60 mL/min 2% B to 26% B in 10 min 8.68.

### Synthetic Procedure of V187178

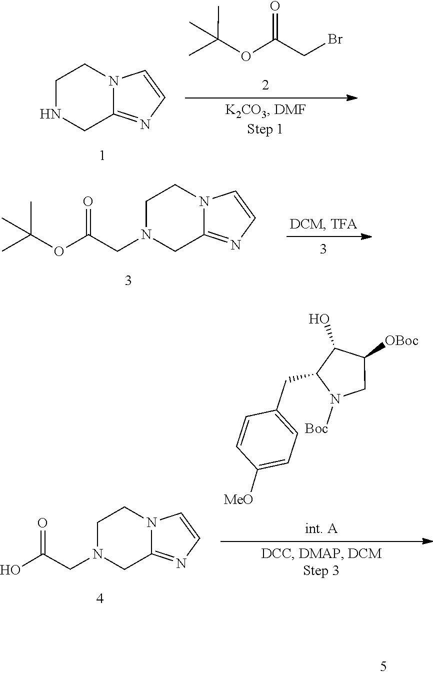

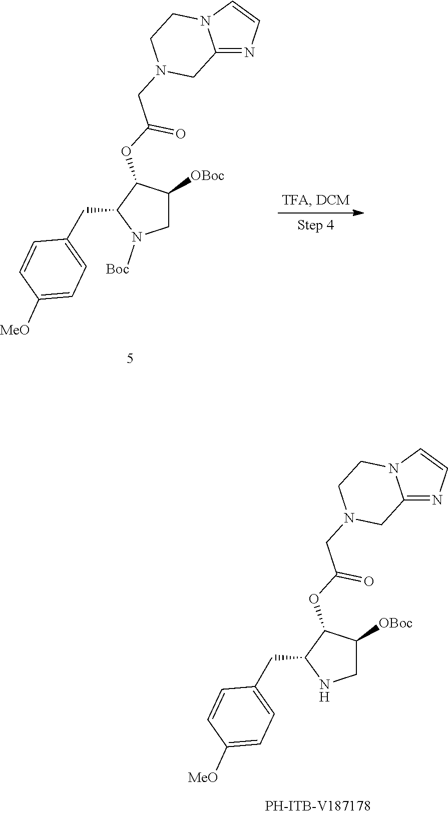

Step 1: To a stirred solution of 5H,6H,7H,8H-imidazo[1,2-a]pyrazine (300 mg, 2.43 mmol, 1 eq.) and tert-butyl 2-bromoacetate (950.2 mg, 4.87 mmol, 2 eq.) in DMF (15 mL) was added K₂CO₃ (1009.9 mg, 7.30 mmol, 3 eq.) at RT. The resulting mixture was stirred at RT for overnight. Upon completion, the reaction mixture was directly purified by a reversed-phase column chromatography (0.05% TFA in water and acetonitrile) to afford tert-butyl 2-{5H,6H,8H-imidazo[1,2-a]pyrazin-7-yl}acetate (120 mg, 20.7%) as a yellow oil. MS: m/z: Calc'd for C₁₂H₁₉N₃O₂ [M+H]⁺238, found 238.

Step 2: To a stirred solution of tert-butyl 2-{5H,6H,8H-imidazo[1,2-a]pyrazin-7-yl}acetate (120 mg, 0.50 mmol) in DCM (9 mL) was added TFA (3 mL). The resulting mixture was stirred for 2 hours. Upon completion, the reaction mixture was concentrated in vacuo. The residue was used in the next step directly without further purification. MS: m/z: Calc'd for C₈H₁₁N₃O₂[M+H]⁺182, found 182.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 80 mg, 0.18 mmol, 1 eq.) and 5H,6H,7H,8H-imidazo[1,2-a]pyrazine (23.2 mg, 0.18 mmol, 1 eq.) in DCM (5 mL) were added DCC (116.9 mg, 0.56 mmol, 3 eq.) and DMAP (34.6 mg, 0.28 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford ert-butyl (2R,3S,4S)-4-[(tert-butoxycarbonyl) oxy]-3-[(2-{5H,6H,8H-imidazo[1,2-a]pyrazin-7-yl}acetyl)oxy]-2-[(4-methoxyphenyl) methyl]pyrrolidine-1-carboxylate (70 mg, 63.1%) as a white solid. MS: m/z: Calc'd for C₃₀H₄₂N₄O₈ [M+H]⁺587, found 587.

Step 4: To a stirred mixture of tert-butyl 2-[(3-{[2-(trimethylsilyl)ethoxy]methyl}-1,2,3-benzotriazol-5-yl)methoxy]acetate (125 mg, 0.31 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford [(3-{[2-(trimethylsilyl)ethoxy]methyl}-1,2,3-benzotriazol-5-yl)methoxy]acetic acid (100 mg, 93.3%) as a white oil. MS: m/z: Calc'd for C₂₀H₂₆N₄₀₄ [M+H]⁺387, found [M+H]⁺387. ¹H NMR (400 MHz, Methanol-d₄) δ 7.52 (s, 2H), 7.31-7.20 (m, 2H), 6.99-6.89 (m, 2H), 5.22-5.15 (m, 1H), 4.45-4.38 (m, 1H), 4.31-4.21 (m, 5H), 3.92-3.82 (m, 2H), 3.80 (s, 3H), 3.64-3.62 (m, 1H), 3.31 (s, 2H), 3.24-3.22 (m, 1H), 3.13-3.08 (m, 1H), 3.00-2.98 (m, 1H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 26% B in 10 min; Wave Length: 254 nm/220 nm nm; RT1(min): 8.68.

### Synthetic Procedure of V187179

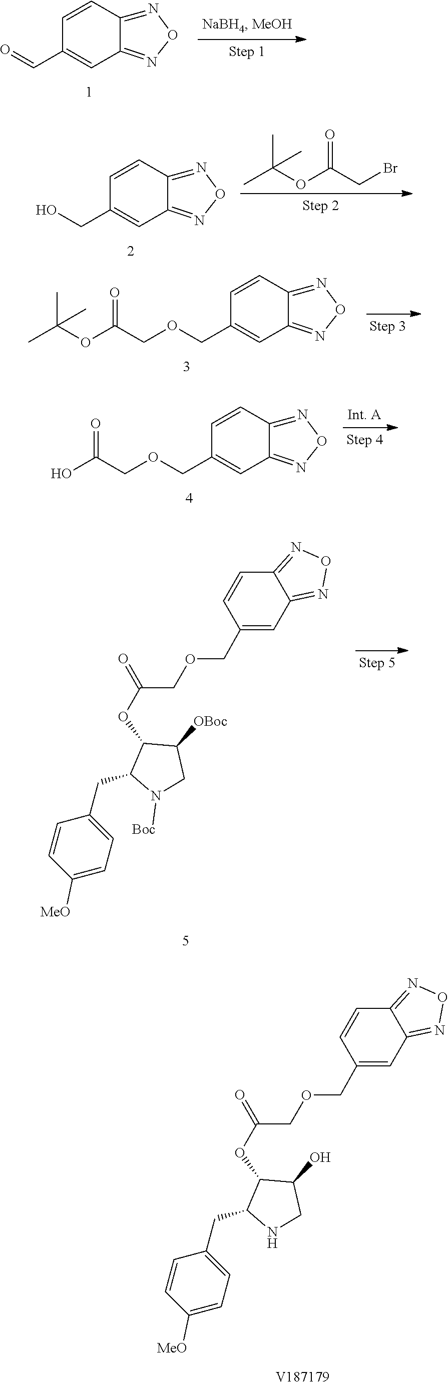

Step 1: To a stirred solution of 2,1,3-benzoxadiazole-5-carbaldehyde (500 mg, 3.37 mmol, 1 eq.) in MeOH (10 mL) was added NaBH₄ (255.4 mg, 6.75 mmol, 2 eq.) at 0° C. The reaction mixture was stirred at room temperature for 2 h. Upon completion, the reaction mixture was concentrated in vacuo. The resulting residue was dissolved in DMSO and purified by a reversed-phase column chromatography (0.05% TFA in water and acetonitrile) to afford 2,1,3-benzoxadiazol-5-ylmethanol (500 mg, 98.6%) as a white solid. MS: m/z: Calc'd for C₁₄H₂₃BrN₂O₂[M−H]⁻149, found 149.

Step 2: To a stirred solution of 2,1,3-benzoxadiazol-5-ylmethanol (400 mg, 2.66 mmol, 1 eq.) in THF (10 mL), was added NaH (191.8 mg, 7.99 mmol, 3 eq.) at 0° C. The mixture was stirred at the same temperature for 30 min. Tert-butyl 2-bromoacetate (519.6 mg, 2.66 mmol, 1 eq.) was added, and the resulting mixture was stirred at room temperature for 2 h. The reaction mixture was concentrated in vacuo. The resulting residue was dissolved in DMSO and purified by a reversed-phase column chromatography (0.05% TFA in water and acetonitrile) to afford tert-butyl 2-(2,1,3-benzoxadiazol-5-ylmethoxy)acetate (170 mg, 24.1%) as a white solid. ¹H NMR (400 MHz, Methanol-d₄) δ 7.97-7.85 (m, 2H), 7.55 (m, 1H), 4.72 (d, J=1.3 Hz, 2H), 4.16 (s, 2H), 1.51 (s, 9H).

Step 3: To a stirred solution of tert-butyl 2-(2,1,3-benzoxadiazol-5-ylmethoxy)acetate (170 mg, 0.64 mmol) in DCM (10 mL) was added TFA (2 mL) and the resulting mixture was stirred at room temperature for 2 h. Desired product could be detected by LCMS. Upon completion, the reaction mixture was concentrated in vacuo to obtain crude product (2,1,3-benzoxadiazol-5-ylmethoxy)acetic acid as a white solid, which was used directly in the next step without further purification. MS: m/z: Calc'd for C₉H₈N₂O₄[M+H]⁺209, found [M+H]⁺209.

Step 4: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 122.0 mg, 0.28 mmol, 1 eq.) and (2,1,3-benzoxadiazol-5-ylmethoxy)acetic acid (120 mg, 0.57 mmol, 2 eq.) in DCM (5 mL) were added DCC (178.4 mg, 0.86 mmol, 3 eq.) and DMAP (69.5 mg, 0.57 mmol, 2 eq.) at 0° C. under nitrogen atmosphere. The resulting reaction mixture was stirred at ambient temperature for overnight.

Upon completion, the reaction mixture was concentrated in vacuo. The resulting residue was dissolved in DMSO and purified by a reversed-phase column chromatography (0.05% TFA in water and acetonitrile) to afford tert-butyl (2R,3S,4S)-3-{[2-(2,1,3-benzoxadiazol-5-ylmethoxy)acetyl]oxy}-4-[(tert-butoxycarbonyl)oxy]-2-[(4-methoxyphenyl) methyl]pyrrolidine-1-carboxylate (70 mg, 19.79%) as a white solid. MS: m/z: Calc'd for C₃₁H₃₉N₃O₁₀ [M+H]⁺614, found 614.

Step 5: To a stirred mixture of tert-butyl (2R,3S,4S)-3-{[2-(2,1,3-benzoxadiazol-5-ylmethoxy)acetyl]oxy}-4-[(tert-butoxycarbonyl)oxy]-2-[(4-methoxyphenyl)methyl]pyrrolidine-1-carboxylate (65 mg, 0.10 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(2,1,3-benzoxadiazol-5-ylmethoxy)acetate (30.0 mg, 67.27%) as a white solid. MS: m/z: Calc'd for C₂₁H₂₃N₃O₆[M+H]⁺414; Found, 414. ¹H NMR (400 MHz, Methanol-d₄) δ 7.96-7.89 (m, 2H), 7.56 (m, 1H), 7.27-7.19 (m, 2H), 6.98-6.89 (m, 2H), 5.21 (d, J=3.5 Hz, 1H), 4.80-4.78 (m, 2H), 4.48-4.38 (m, 3H), 4.25 (m, 1H), 3.80 (s, 3H), 3.59-3.57 (m, 1H), 3.27-3.19 (m, 1H), 3.11-3.09 (m, 1H), 2.96-2.94 (m, 1H).

Prep-HPLC-conditions: Column: XSelect CSH Prep C18 OBD Column, 5 μm, 30*150 mm; Mobile Phase A:Water (0.05% TFA), Mobile Phase B:ACN; Flow rate:60 mL/min; Gradient:13 B to 43 B in 12 min; 254/220 nm; RT1:8.62; RT2:;Injection Volumn: ml; Number Of Runs:;

### Synthetic Procedure of V187181

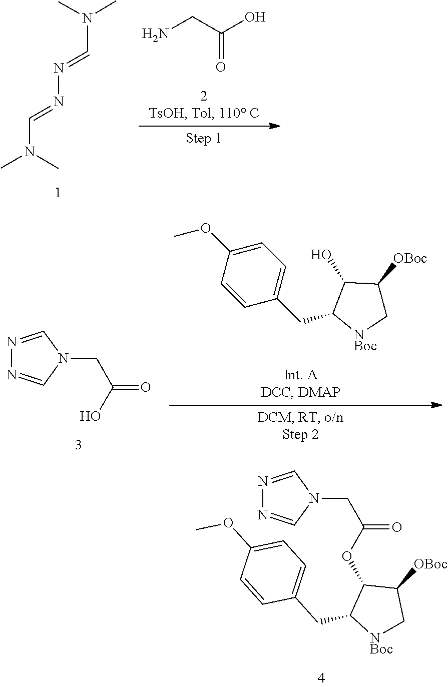

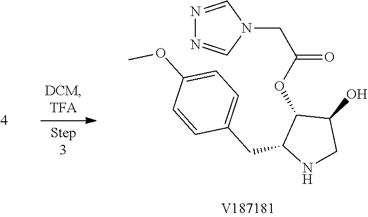 [SMILES: COc1ccc(C[C@H]2NC[C@H](O)[C@H]2OC(=O)Cn2cnnc2)cc1]

Step 1: To a stirred solution of (E)-N′-[(E)-N′-[(N,N-dimethylamino)methylidene]amino]-N,N-dimethylmethanimidamide (2.84 g, 19.98 mmol, 1.5 eq) and glycine (1 g, 13.32 mmol, 0.8 eq) in toluene (3 mL) was added TsOH (69 mg, 0.34 mmol, 0.02 eq) at room temperature under nitrogen atmosphere. The resulting mixture was stirred at 110° C. for 12 h. The mixture was basified to pH 9 with KOH (EtOH) and stirred for 5 min. The mixture was then acidified to pH 4 with HCl (aq·EtOH). The precipitated solids were collected by filtration and washed with EtOH (3×10 mL). This resulted in 1,2,4-triazol-4-ylacetic acid (1.1 g, 65.%) as a yellow solid. MS: m/z: Calc'd for C₄H₅N₃O₂ [M+H]⁺128, found 128.

Step 2: To a stirred solution of 1,2,4-triazol-4-ylacetic acid (45 mg, 0.35 mmol, 1.5 eq) in DCM (5 mL) was added DCC (73 mg, 0.35 mmol, 1.5 eq), DMAP (29 mg, 0.24 mmol, 1 eq) and tert-butyl (2R,3S,4S)-4-[(tert-butoxycarbonyl)oxy]-3-hydroxy-2-[(4-methoxyphenyl)methyl]pyrrolidine-1-carboxylate (100 mg, 0.24 mmol, 1 eq) at 0° C. under nitrogen atmosphere. The resulting mixture was stirred at 0° C. for 12 h. The mixture was concentrated and the residue was purified by reversed-phase flash chromatography to afford tert-butyl (2R,3S,4S)-4-[(tert-butoxycarbonyl)oxy]-2-[(4-methoxyphenyl)methyl]-3-{[2-(1,2,4-triazol-4-yl)acetyl]oxy}pyrrolidine-1-carboxylate (120 mg, 95.4%) as a light yellow solid. MS: m/z: Calc'd for C₂₆H₃₆N₄O₅[M+H]⁺533, found 533.

Step 3: To a stirred solution of tert-butyl (2R,3S,4S)-4-[(tert-butoxycarbonyl)oxy]-2-[(4-methoxyphenyl)methyl]-3-{[2-(1,2,4-triazol-4-yl)acetyl]oxy}pyrrolidine-1-carboxylate (120 mg, 0.23 mmol, 1 eq) in DCM (5 mL) was added TFA (1 mL) dropwise at 0° C. under nitrogen atmosphere. The resulting mixture was stirred at room temperature for 2 h. The mixture was concentrated and the crude product (70 mg) was purified by Prep-HPLC to afford (2R,3S,4S)-4-hydroxy-2-[(4-methoxyphenyl) methyl]pyrrolidin-3-yl 2-(1,2,4-triazol-4-yl)acetate (33.6 mg, 58.5%) as a white solid. MS: m/z: Calc'd for C₁₆H₂₀N₄O₄ [M+H]⁺333, found 333. ¹H NMR (400 MHz, Methanol-d4) δ 8.64 (s, 2H), 7.27 (d, J=8.2 Hz, 2H), 7.00-6.92 (m, 2H), 5.34-5.16 (m, 3H), 4.49 (d, J=4.2 Hz, 1H), 4.31-4.24 (m, 1H), 3.81 (s, 3H), 3.61 (dd, J=12.8, 4.3 Hz, 1H), 3.27 (d, J=12.7 Hz, 1H), 3.13 (dd, J=14.5, 6.1 Hz, 1H), 2.97 (dd, J=14.5, 9.4 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 15% B in 10 min; Wave Length: 254 nm/220 nm nm; RT1(min): 8.68

### Synthetic Procedure of V187182

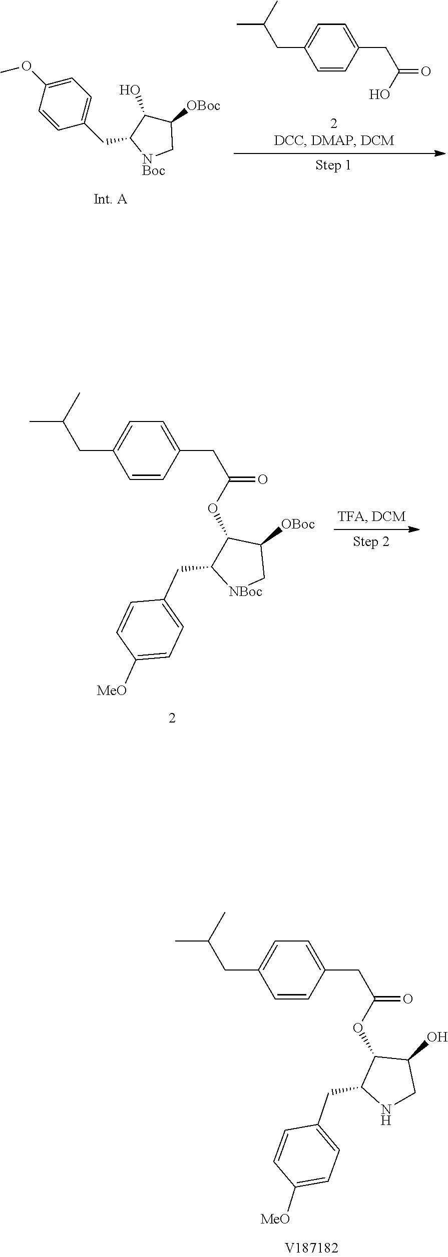

Step 1: A solution of 2-(4-isobutylphenyl)acetic acid (73 mg, 0.37 mmol, 2 equiv) in DCM (1 mL) was treated with DCC (58.4 mg, 0.28 mmol, 1.5 equiv) and DMAP (23.0 mg, 0.18 mmol, 1 equiv) at 0° C. under nitrogen atmosphere followed by the addition of tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.18 mmol, 1 equiv). The resulting mixture was stirred for overnight at room temperature and directly purified by Prep-TLC (PE/EA 7:1) to afford tert-butyl (2R,3S,4S)-4-[(tert-butoxycarbonyl)oxy]-2-[(4-methoxyphenyl)methyl]-3-({2-[4-(2-methylpropyl)phenyl]acetyl}oxy)pyrrolidine-1-carboxylate (80 mg, 66.6%) as a colorless oil. MS: m/z: Calc'd for C₂₅H₃₄FNO₈[M+H−56−56]⁺486, found 486.

Step 2: To a stirred solution of tert-butyl (2R,3S,4S)-4-[(tert-butoxycarbonyl)oxy]-2-[(4-methoxyphenyl)methyl]-3-({2-[4-(2-methylpropyl)phenyl]acetyl}oxy)pyrrolidine-1-carboxylate (80 mg, 0.13 mmol) in DCM (2.5 mL) was added TFA (0.5 mL) at 0° C. The resulting mixture was stirred for 1 h at room temperature and concentrated under reduced pressure. The crude product (50 mg) was purified by Prep-HPLC to afford (2R,3S,4S)-4-hydroxy-2-[(4-methoxy phenyl)methyl]pyrrolidin-3-yl 2-[4-(2-methylpropyl)phenyl]acetate; trifluoroacetic acid (22.4 mg, 32.5%) as a white solid. MS: m/z: Calc'd for C₂₅H₃₄FNO₈[M+H]⁺398, found 398. ¹H NMR (400 MHz, Methanol-d₄) δ 7.30 (d, J=8.0 Hz, 2H), 7.19 (d, J=7.9 Hz, 2H), 7.01-6.92 (m, 2H), 6.86-6.78 (m, 2H), 4.98 (d, J=3.4 Hz, 1H), 4.34-4.29 (m, 1H), 4.15-4.02 (m, 1H), 3.76 (d, J=7.3 Hz, 5H), 3.55 (dd, J=12.6, 4.3 Hz, 1H), 3.22 (d, J=12.6 Hz, 1H), 2.89-2.76 (m, 2H), 2.49 (d, J=7.2 Hz, 2H), 1.85-1.72 (m, 1H), 0.88 (dd, J=6.6, 3.4 Hz, 6H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 23% B to 53% B in 10 min; Wave Length: 254 nm/220 nm nm; RT1(min): 8.68

### Synthetic Procedure of V187184

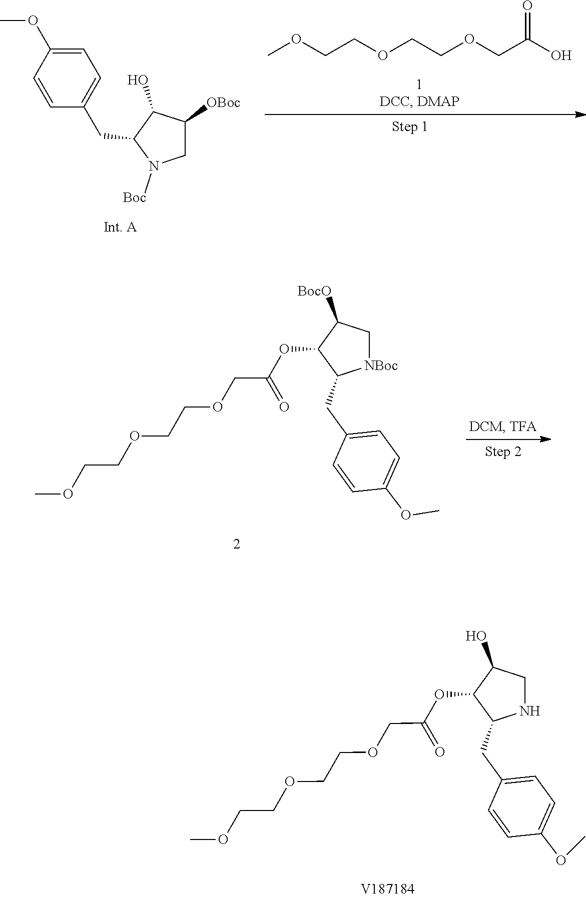

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and (4,4-difluorocyclohexyl)acetic acid (84.1 mg, 0.47 mmol, 2 eq.) in DCM (5 mL) were added DCC (73.0 mg, 0.35 mmol, 1.5 eq.) and DMAP (28.8 mg, 0.23 mmol, 1 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-3-{[2-(4,4-difluorocyclohexyl)acetyl]oxy}-2-[(4-methoxyphenyl)methyl]pyrrolidine-1-carboxylate (65 mg, 47.1%). MS: m/z: Calc'd for C₃₀H₄₃F₂NO₈ [M−H]⁻582, found 582.

Step 2: To a stirred mixture of tert-butyl (2R,3S,4S)-4-[(tert-butoxycarbonyl)oxy]-3-({2-[2-(2-methoxyethoxy)ethoxy]acetyl}oxy)-2-[(4-methoxyphenyl)methyl]pyrrolidine-1-carboxylate (65 mg, 0.11 mmol, 1 eq.) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-[2-(2-methoxyethoxy)ethoxy]acetate (15.1 mg, 34.9%) as a white solid. MS: m/z: Calc'd for C₁₉H₂₉NO₇ [M+H]⁺384; Found, 384. ¹H NMR (400 MHz, Methanol-d₄) δ 77.28-7.20 (m, 2H), 6.98-6.90 (m, 2H), 5.16 (d, J=3.3 Hz, 1H), 4.43-4.36 (m, 1H), 4.34 (d, J=4.6 Hz, 2H), 4.23 (m, 1H), 3.82-3.77 (m, 3H), 3.74-3.63 (m, 2H), 3.69-3.67 (m, 2H), 3.66-3.55 (m, 2H), 3.59-3.57 (m, 1H), 3.56-3.55 (m, 2H), 3.37 (s, 3H), 3.23 (d, J=12.7 Hz, 1H), 3.11-3.09 (m, 1H), 2.97-2.95 (m, 1H). Prep-HPLC-conditions: Column: XSelect CSH Prep C18 OBD Column, 5 μm, 19*150 mm; Mobile Phase A:Water (0.05% TFA), Mobile Phase B:ACN; Flow rate:60 mL/min; Gradient:2 B to 30 B in 10 min; 254/220 nm; RT1:8.68; RT2:8.6; Injection Volumn: ml; Number Of Runs:;

### Synthetic Procedure of V187324

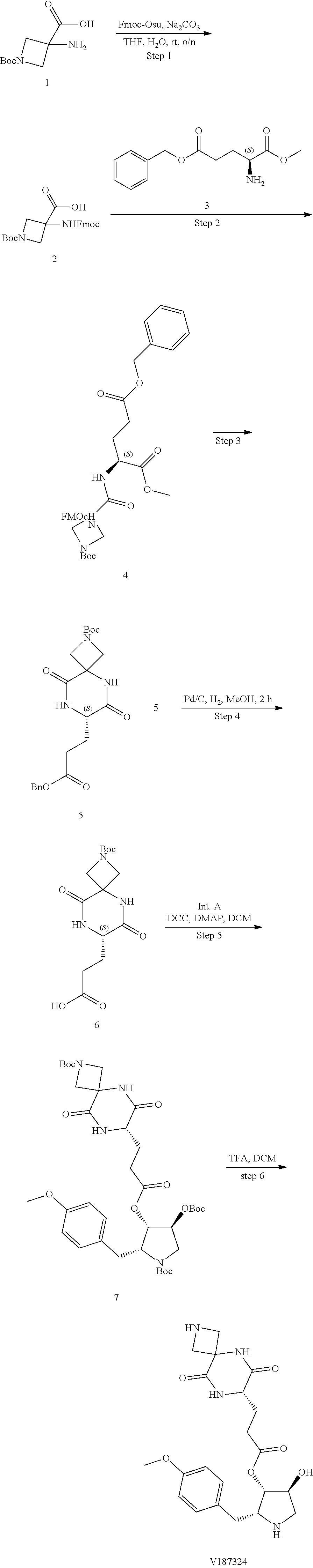

Step 1: To a stirred solution of 3-amino-1-(tert-butoxycarbonyl)azetidine-3-carboxylic acid (500 mg, 2.31 mmol, 1 eq.) in H₂O (5 mL) and THF (5 mL) were added 2,5-dioxopyrrolidin-1-yl (9H-fluoren-9-yl)methyl carbonate (1949.9 mg, 5.78 mmol, 2.5 eq.) and Na₂CO₃ (735.2 mg, 6.93 mmol, 3 eq.) at 0° C. The reaction was stirred at r.t. for overnight. After completion of the reaction monitored by LCMS, the resulting mixture was extracted with EtOAc (3×100 mL). The combined organic layers were washed with water (2×100 mL), dried over anhydrous Na₂SO₄. After filtration, the filtrate was concentrated under reduced pressure. The crude product was purified by reverse phase flash to afford 1-(tert-butoxycarbonyl)-3-{[(9H-fluoren-9-ylmethoxy)carbonyl]amino}azetidine-3-carboxylic acid (1.5 g, 147.9%) as a yellow oil. MS: m/z: Calc'd for C₂₄H₂₆N₂O₆[M+H−100]⁺339; Found, 339.

Step 2: To a stirred solution of 1-(tert-butoxycarbonyl)-3-{[(9H-fluoren-9-ylmethoxy) carbonyl]amino}azetidine-3-carboxylic acid (500 mg, 1.14 mmol, 1 eq.) and 5-benzyl 1-methyl (2S)-2-aminopentanedioate (286.5 mg, 1.14 mmol, 1 eq.) in DMF (5 mL) were added HATU (650.3 mg, 1.71 mmol, 1.5 eq.) and DIEA (442.1 mg, 3.42 mmol, 3 eq.) at 0° C. The reaction was stirred at r.t. for overnight. After completion of the reaction monitored by LCMS, the resulting mixture was extracted with EtOAc (3×100 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (1:1) to afford 5-benzyl 1-methyl (2S)-2-{[1-(tert-butoxy carbonyl)-3-{[(9H-fluoren-9-ylmethoxy)carbonyl]amino}azetidin-3-yl]formamido}pentanedioate (360 mg, 47.0%) as a white solid. MS: m/z: Calc'd for ₃₇H₄₁N₃₀₉[M+H⁺22]⁺694; Found, 694.

Step 3: To a stirred solution of 5-benzyl 1-methyl (2S)-2-{[1-(tert-butoxycarbonyl)-3-{[(9H-fluoren-9-ylmethoxy)carbonyl]amino}azetidin-3-yl]formamido}pentanedioate (650 mg, 0.96 mmol, 1 eq.) in DMF (7 mL) were added piperidine (247.1 mg, 2.90 mmol, 3 eq.) at r.t. The reaction was stirred at r.t. for overnight. After completion of the reaction monitored by LCMS, the crude product was purified by reverse phase flash with the following conditions (05% NH₄HCO₃ in H₂O/ACN)) to afford 5-benzyl 1-methyl (2S)-2-{[3-amino-1-(tert-butoxycarbonyl) azetidin-3-yl]formamido}pentanedioate (320 mg, 73.5%) as a white solid. MS: m/z: Calc'd for C₂₁H₂₇N₃O₆ [M+H−100]⁺318; Found, 318.

Step 4: Under a nitrogen atmosphere, Pd/C (100 mg, 0.94 mmol, 1.96 eq.) was added to a solution of tert-butyl (7S)-7-[3-(benzyloxy)-3-oxopropyl]-6,9-dioxo-2,5,8-triazaspiro[3.5]nonane-2-carboxylate (200 mg, 0.09 mmol, 1 eq.) in MeOH (5 mL), H₂ was subsequently introduced into the reaction system, and the resulting mixture was stirred at r.t. for 2 h. The reaction mixture was filtered to obtain 3-[(7S)-2-(tert-butoxycarbonyl)-6,9-dioxo-2,5,8-triazaspiro[3.5]nonan-7-yl]propanoic acid (150 mg, 95.6%) as a white solid. MS: m/z: Calc'd for C₁₄H₂₁N₃O₆[M−H]⁻326; Found, 326.

Step 5: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.23 mmol, 1 eq.) and 3-[(7S)-2-(tert-butoxycarbonyl)-6,9-dioxo-2,5,8-triazaspiro[3.5]nonan-7-yl]propanoic acid (154.5 mg, 0.47 mmol, 2 eq.) in DCM (3 mL) were added DCC (73.0 mg, 0.35 mmol, 1.5 eq.) and DMAP (28.8 mg, 0.23 mmol, 1 eq.) at 0° C. The reaction was stirred at r.t. for overnight. After completion of the reaction monitored by LCMS. The reaction mixture was quenched with water and extracted with EA. The extracts were washed with brine, dried over anhydrous sodium sulfate, filtered and concentrated. The residue was purified by Prep-HPLC to afford tert-butyl 7-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)-6,9-dioxo-2,5,8-triazaspiro[3.5]nonane-2-carboxylate (100 mg, 44.6%) as a white solid.

MS: m/z: Calc'd for C₃₆H₅₂N₄O₁₂[M+H⁺22]⁺755; Found, 755.

Step 6: To a stirred solution of tert-butyl (7S)-7-(3-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-3-oxopropyl)-6,9-dioxo-2,5,8-triazaspiro[3.5]nonane-2-carboxylate (80 mg, 0.10 mmol, 1 eq.) in DCM (3 mL) was added TFA (1 mL) dropwise at 0° C. The reaction was stirred at r.t. for 2 h. The resulting mixture was concentrated under reduced pressure. The crude product (80 mg) was purified by Prep-HPLC to afford (2R,3S,4S)-4-hydroxy-2-[(4-methoxyphenyl)methyl]pyrrolidin-3-yl 3-[(7S)-6,9-dioxo-2,5,8-triazaspiro[3.5]nonan-7-yl]propanoate (25.4 mg, 52.9%) as a white solid. MS: m/z: Calc'd for C₂₁H₂₈N₄O₆[M+H]⁺433; Found, 433. ¹H NMR (400 MHz, DMSO-d₆) δ 9.64 (s, 1H), 9.29 (s, 1H), 9.13 (s, 2H), 9.09 (s, 1H), 8.74-8.69 (m, 1H), 7.24-7.21 (m, 2H), 6.94-6.87 (m, 2H), 6.03 (s, 1H), 4.89 (d, J=3.4 Hz, 1H), 4.32-4.20 (m, 3H), 4.10-4.06 (m, 2H), 4.05-3.97 (m, 2H), 3.74 (s, 3H), 3.48 (d, J=12.4 Hz, 1H), 3.08 (d, J=12.5 Hz, 1H), 3.02-2.81 (m, 2H), 2.62-2.51 (m, 1H), 2.50-2.45 (m, 1H), 2.01-1.98 (m, 2H). Prep-HPLC-conditions: Column: Welch Ultimate XB-C18 50*250, 10 μm; Mobile Phase A: Water(0.05% TFA), Mobile Phase B: ACN; Flow rate: 60 mL/min mL/min; Gradient: 1% B to 5% B in 10 min; Wave Length: 254 nm/220 nm nm; RT1(min): 8.18

### Synthetic Procedure of V187332

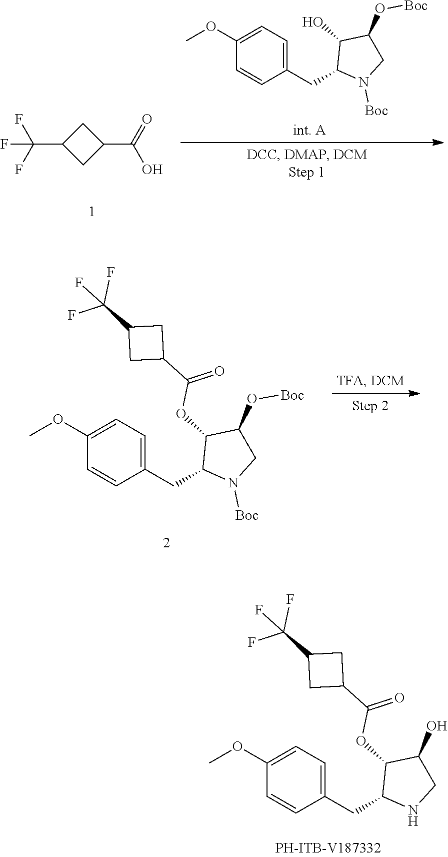

Step 1: To solution of 3-(trifluoromethyl)cyclobutane-1-carboxylic acid (63.5 mg, 0.37 mmol, 2 equiv) in DCM (10 mL) was treated with tert-butyl (2R,3S,4S)-4-[(tert-butoxycarbonyl)oxy]-3-hydroxy-2-[(4-methoxyphenyl)methyl]pyrrolidine-1-carboxylate (80 mg, 0.18 mmol, 1 equiv), DCC (116.9 mg, 0.56 mmol, 3 equiv) and DMAP (34.6 mg, 0.28 mmol, 1.5 equiv) at 0° C. The resulting mixture was stirred for 2 hours at RT. Upon completion, the reaction mixture was concentrated in vacuo. The residue was purified by Prep-TLC (PE/EA) to afford tert-butyl tert-butyl (2R,3S,4S)-4-[(tert-butoxycarbonyl)oxy]-2-[(4-methoxyphenyl)methyl]-3-[3-(trifluoromethyl)cyclobutanecarbonyloxy]pyrrolidine-1-carboxylate (80 mg, 74.1%) as a yellow oil.

MS: m/z: Calc'd for C₂₈H₃₈F₃NO₈ [M+H−56−56]⁺462, found 462.

Step 2: To a solution of tert-butyl (2R,3S,4S)-4-[(tert-butoxycarbonyl)oxy]-2-[(4-methoxyphenyl) methyl]-3-[3-(trifluoromethyl)cyclobutanecarbonyloxy]pyrrolidine-1-carboxylate (70 mg, 0.122 mmol) in DCM (9 mL) was added TFA (3 mL). The mixture was stirred for 2 h at RT. Upon completion, the reaction mixture was concentrated in vacuo. The residue was purified by Prep-HPLC to afford (2R,3S,4S)-4-hydroxy-2-[(4-methoxyphenyl) methyl]pyrrolidin-3-yl 3-(trifluoromethyl)cyclobutane-1-carboxylate (16 mg, 33.4%) as a white solid. MS: m/z: Calc'd for C₁₈H₂₂F₃NO₄ [M+H]⁺374, found 374. ¹H NMR (400 MHz, Methanol-d₄) δ 7.17-7.05 (m, 2H), 6.88-6.79 (m, 2H), 4.82-4.78 (m, 1H), 4.13-4.05 (m, 1H), 3.77 (s, 3H), 3.55 (d, J=7.6, 1H), 3.38-3.33 (m, 1H), 3.27 (d, J=9.1 Hz, 1H), 3.09-3.07 (m, 1H), 2.81-2.65 (m, 3H), 2.52-2.33 (m, 4H). Prep-HPLC conditions: Column: Column: XBridge Prep OBD C18 Column, 30*150 mm, 5 m; Mobile Phase A: Water (10 mmol/L NH₄HCO₃), Mobile Phase B: ACN; Flow rate: 60 mL/min mL/min; Gradient: 27% B to 46% B in 8 min; Wave Length: 254 nm/220 nm nm; RT1(min): 6.2

### Synthetic Procedure of V187333

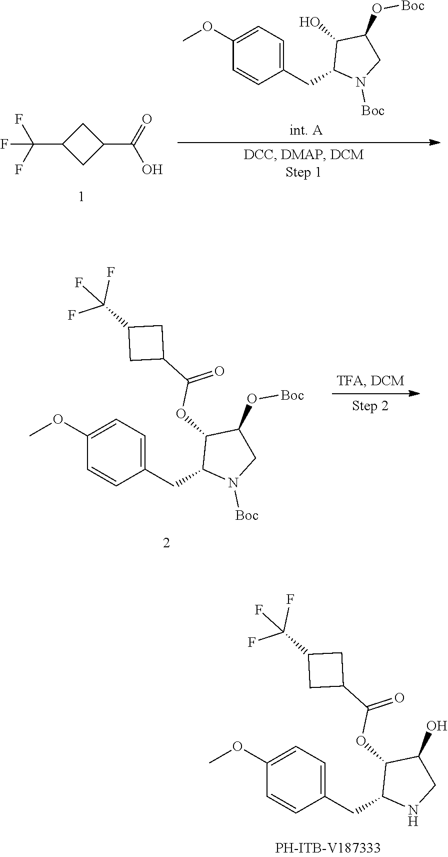

Step 1: To a solution of 3-(trifluoromethyl)cyclobutane-1-carboxylic acid (63.5 mg, 0.37 mmol, 2 equiv) in DCM (10 mL) was treated with tert-butyl (2R,3S,4S)-4-[(tert-butoxy carbonyl)oxy]-3-hydroxy-2-[(4-methoxyphenyl)methyl]pyrrolidine-1-carboxylate (80 mg, 0.18 mmol, 1 equiv), DCC (116.9 mg, 0.56 mmol, 3 equiv) and DMAP (34.62 mg, 0.283 mmol, 1.5 equiv) at 0° C. The resulting mixture was stirred for 2 hours at RT. Upon completion, the reaction mixture was concentrated in vacuo. The residue was purified by Prep-TLC (PE/EA) to afford tert-butyl tert-butyl (2R,3S,4S)-4-[(tert-butoxycarbonyl)oxy]-2-[(4-methoxyphenyl) methyl]-3-[3-(trifluoromethyl)cyclobutanecarbonyloxy]pyrrolidine-1-carboxylate (80 mg, 74.1%) as a yellow oil.

MS: m/z: Calc'd for C₂₈H₃₈F₃NO₈[M+H−56−56]⁺462, found 462.

Step 2: To a solution of tert-butyl (2R,3S,4S)-4-[(tert-butoxycarbonyl)oxy]-2-[(4-methoxyphenyl) methyl]-3-[3-(trifluoromethyl)cyclobutanecarbonyloxy]pyrrolidine-1-carboxylate (70 mg, 0.122 mmol) in DCM (9 mL) was added TFA (3 mL). The mixture was stirred for 2 hours at RT. Upon completion, the reaction mixture was concentrated in vacuo. The residue was purified by Prep-HPLC to afford (2R,3S,4S)-4-hydroxy-2-[(4-methoxyphenyl)methyl]pyrrolidin-3-yl 3-(trifluoromethyl)cyclobutane-1-carboxylate (12.3 mg, 26.7%) as a white solid. MS: m/z: Calc'd for C₁₈H₂₂F₃NO₄ [M+H]⁺374, found 374. ¹H NMR (400 MHz, Methanol-d₄) δ 7.13 (d, J=8.7, 2H), 6.86 (d, J=12.5, 2H), 4.21-4.09 (m, 1H), 3.78-3.76 (m, 3H), 3.70-3.59 (m, 1H), 3.42-3.34 (m, 2H), 3.20-3.06 (m, 1H), 2.82-2.80 (m, 3H), 2.61-2.41 (m, 4H). Prep-HPLC conditions: Column: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 27% B to 46% B in 8 min; Wave Length: 254 nm/220 nm nm; RT1(min): 7.5

### Synthetic Procedure of V187368

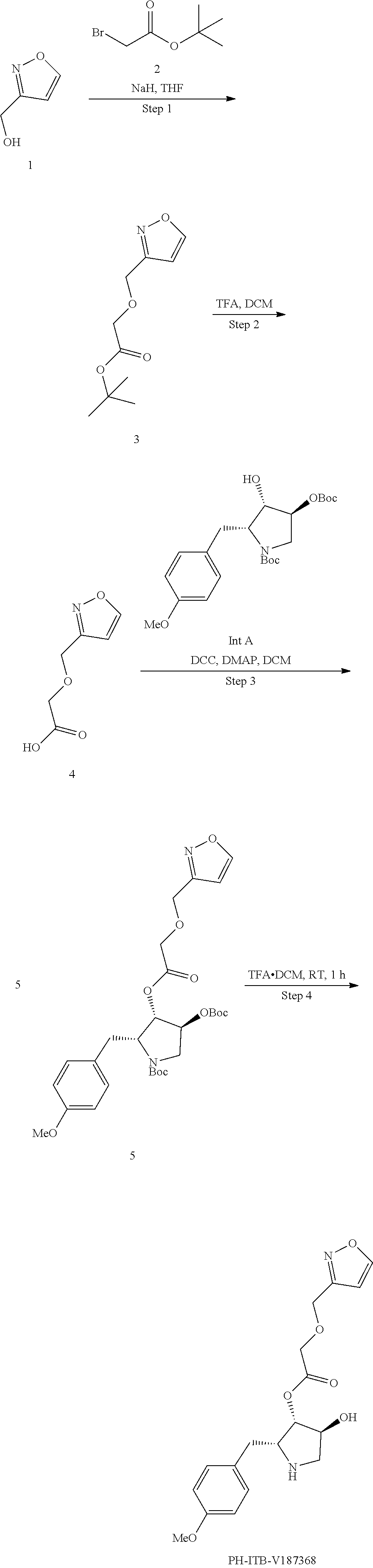

Step 1: A solution of 1,2-oxazol-3-ylmethanol (200 mg, 2.01 mmol, 1 equiv.) in THF (5 mL) was treated with NaH (145.3 mg, 6.05 mmol, 3.00 equiv) for 0.5 h at 0° C. under nitrogen atmosphere followed by the addition of tert-butyl 2-bromoacetate (1181.1 mg, 6.05 mmol, 3 equiv.). The resulting mixture was stirred for overnight at room temperature under nitrogen atmosphere. The reaction was quenched by the addition of water (10 mL) at 0° C. The aqueous phase was extract with EA, dried and concentrated. The residue was purified by reversed-phase flash chromatography with the following conditions: column, C18 silica gel; mobile phase, MeCN in Water (0.1% TFA), 30% to 50% gradient in 10 min; detector, UV 254 nm. This resulted in tert-butyl 2-(1,2-oxazol-3-ylmethoxy)acetate (250 mg, 58.1%) as a yellow oil.

MS: m/z: Calc'd for C₁₀H₁₅NO₄ [M+H−56]⁺158, found 158.

Step 2: To a stirred solution of tert-butyl 2-(1,2-oxazol-3-ylmethoxy)acetate (250 mg, 1.17 mmol, 1 equiv) in DCM (5 mL) was added TFA (1 mL) at 0° C. under N₂ atmosphere. The resulting mixture was stirred for 1 h at room temperature. The mixture was concentrated under vacuum to afford (1,2-oxazol-3-ylmethoxy)acetic acid (210 mg, 94.1%) as a yellow oil.

MS: m/z: Calc'd for C₆H₇NO₄ [M+H]⁺158, found 158.

Step 3: A solution of (1,2-oxazol-3-ylmethoxy)acetic acid (74 mg, 0.47 mmol, 2 equiv) in DCM (5 mL) was treated with DCC (73 mg, 0.35 mmol, 1.5 equiv) and DMAP (29 mg, 0.23 mmol, 1 equiv) at 0° C. under nitrogen atmosphere followed by the addition of tert-butyl (2R,3S,4S)-4-[(tert-butoxycarbonyl) oxy]-3-hydroxy-2-[(4-methoxyphenyl)methyl]pyrrolidine-1-carboxylate (100 mg, 0.23 mmol, 1.00 equiv.). The resulting mixture was stirred for overnight at room temperature. The resulting mixture was filtered, the filter cake was washed with CH₂Cl₂ (2×20 mL).

The filtrate was concentrated under reduced pressure and the resulting residue was purified by Prep-TLC (PE/EA 1:1) to afford tert-butyl (2R,3S,4S)-4-[(tert-butoxycarbonyl)oxy]-2-[(4-methoxyphenyl)methyl]-3-{[2-(1,2-oxazol-3-ylmethoxy)acetyl]oxy}pyrrolidine-1-carboxylate (36 mg, 27.1%) as a yellow oil.

MS: m/z: Calc'd for C₂₈H₃₈N₂O₁₀ [M+H⁺22]⁺585, found 585.

Step 4: To a stirred solution of tert-butyl (2R,3S,4S)-4-[(tert-butoxycarbonyl)oxy]-2-[(4-methoxyphenyl)methyl]-3-{[2-(1,2-oxazol-3-ylmethoxy)acetyl]oxy}pyrrolidine-1-carboxylate (36 mg, 0.06 mmol, 1 equiv) in DCM (5 mL) was added TFA (1 mL) at 0° C. under N₂ atmosphere. The resulting mixture was stirred at room temperature for 1 hour. Desired product could be detected by LCMS. The resulting mixture was concentrated under reduced pressure and the crude product (30 mg) was purified by Prep-HPLC to afford (2R,3S,4S)-4-hydroxy-2-[(4-methoxyphenyl)methyl]pyrrolidin-3-yl 2-(1,2-oxazol-3-ylmethoxy)acetate (18.3 mg, 78.8%) as a white solid. MS: m/z: Calc'd for C₁₈H₂₂N₂O₆ [M+H]⁺363, found 363. ¹H NMR (400 MHz, Methanol-d₄) δ 8.69 (d, J=1.7 Hz, 1H), 7.28-7.20 (m, 2H), 6.98-6.90 (m, 2H), 6.61 (d, J=1.7 Hz, 1H), 5.20-5.14 (m, 1H), 4.80 (s, 2H), 4.44-4.39 (m, 1H), 4.39-4.29 (m, 2H), 4.24-4.12 (m, 1H), 3.80 (s, 3H), 3.60 (dd, J=12.7, 4.4 Hz, 1H), 3.23 (d, J=12.7 Hz, 1H), 3.09 (dd, J=14.3, 6.8 Hz, 1H), 2.97 (dd, J=14.3, 8.8 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.68

### Synthetic Procedure of V187517

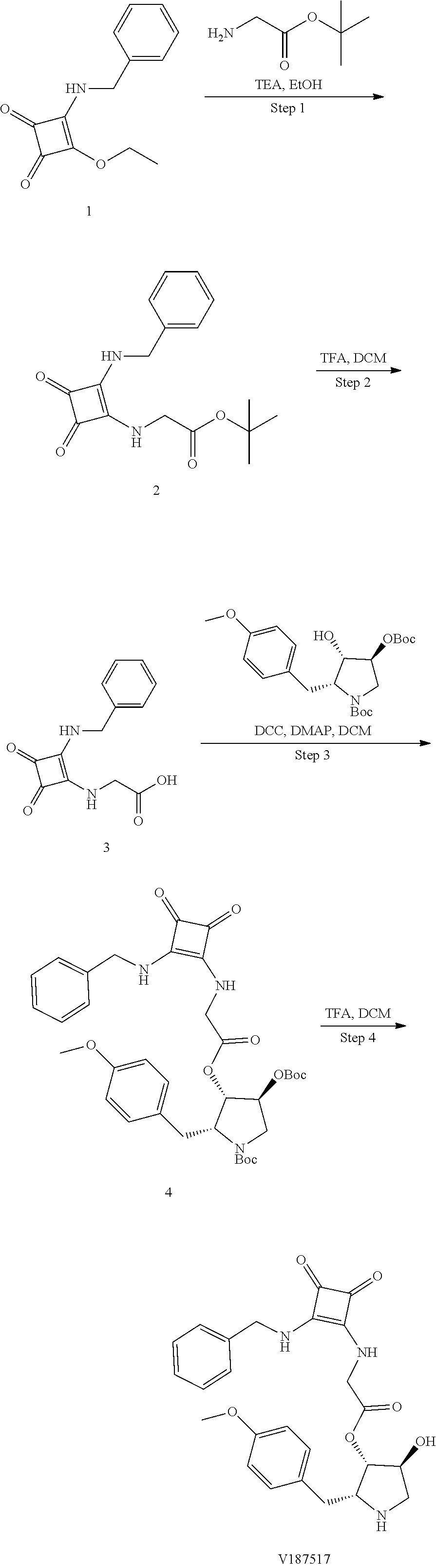

Step 1: To a stirred solution of 3-(benzylamino)-4-ethoxycyclobut-3-ene-1,2-dione (250 mg, 1.08 mmol, 1 eq.) and tert-butyl 2-aminoacetate (283.6 mg, 2.16 mmol, 2 eq.) in EtOH (6 mL) was added TEA (328.2 mg, 3.24 mmol, 3 eq.) at room temperature and the resulting mixture was stirred for overnight. Then the mixture was filtered, filter cake was dried in vacuo to obtain tert-butyl 2-{[2-(benzylamino)-3,4-dioxocyclobut-1-en-1-yl]amino}acetate (200 mg, 58.5%) as a white solid. MS: m/z: Calc'd for C₁₇H₂₀N₂O₄[M+H−56]⁺261, found 261.

Step 2: To a stirred solution of tert-butyl 2-{[2-(benzylamino)-3,4-dioxocyclobut-1-en-1-yl]amino}acetate (190 mg, 0.60 mmol, 1 eq.) in DCM (5 mL) was added TFA (1 mL) at room temperature for 2 h. Then the mixture was concentrated to obtain {[2-(benzylamino)-3,4-dioxocyclobut-1-en-1-yl]amino}acetic acid (150 mg, 95.9%) as a yellow oil. MS: m/z: Calc'd for C₁₃H₁₂N₂O₄ [M−H]⁻259, found 259.

Step 3: To a stirred solution of {[2-(benzylamino)-3,4-dioxocyclobut-1-en-1-yl]amino}acetic acid (120 mg, 0.46 mmol, 1 eq.), DCC (190.2 mg, 0.92 mmol, 2 eq.) and DMAP (84.5 mg, 0.69 mmol, 1.5 eq.) in DCM (10 mL) was added tert-butyl (2R,3S,4S)-4-[(tert-butoxycarbonyl)oxy]-3-hydroxy-2-[(4-methoxyphenyl)methyl]pyrrolidine-1-carboxylate (Int A, 195.2 mg, 0.46 mmol, 1 eq.) at room temperature and the resulting mixture was stirred at room temperature for overnight. H₂O was added the mixture and extracted with DCM, the organic layer was concentrated and purified by column chromatography to obtain tert-butyl (2R,3S,4S)-3-[(2-{[2-(benzylamino)-3,4-dioxocyclobut-1-en-1-yl]amino}acetyl)oxy]-4-[(tert-butoxycarbonyl)oxy]-2-[(4-methoxyphenyl)methyl]pyrrolidine-1-carboxylate (90 mg, 29.3%) as a yellow solid. MS: m/z: Calc'd for C₃₅H₄₃N₃O₁₀ [M+H−100-56]⁺510, found 510.

Step 4: To a stirred solution of tert-butyl (2R,3S,4S)-3-[(2-{[2-(benzylamino)-3,4-dioxocyclobut-1-en-1-yl]amino}acetyl)oxy]-4-[(tert-butoxycarbonyl)oxy]-2-[(4-methoxy phenyl)methyl]pyrrolidine-1-carboxylate (80 mg, 0.12 mmol, 1 eq.) in DCM (10 mL) was added TFA (2 mL) at room temperature and the resulting mixture was stirred for 2 h. The crude was concentrated and purified by Prep-HPLC to obtain (2R,3S,4S)-4-hydroxy-2-[(4-methoxy phenyl)methyl]pyrrolidin-3-yl 2-{[2-(benzylamino)-3,4-dioxocyclobut-1-en-1-yl]amino}acetate; trifluoroacetic acid (20.4 mg, 29.0%) as a white solid. MS: m/z: Calc'd for C₂₅H₂₇N₃O₆[M+H]⁺466, found 466. ¹H NMR (400 MHz, Methanol-d₄) δ 7.39 (d, J=4.4 Hz, 4H), 7.37-7.28 (m, 1H), 7.28-7.22 (m, 2H), 6.97-6.90 (m, 2H), 5.17-5.12 (m, 1H), 4.83 (s, 2H), 4.53 (s, 2H), 4.43 (d, J=4.2 Hz, 1H), 4.23-4.18 (m, 1H), 3.79 (s, 3H), 3.64 (dd, J=12.7, 4.4 Hz, 1H), 3.24 (d, J=12.7 Hz, 1H), 3.11 (dd, J=14.3, 6.8 Hz, 1H), 3.00 (dd, J=14.3, 8.8 Hz, 1H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm nm; RT1(min): 8.5

### Synthetic Procedure of V187533

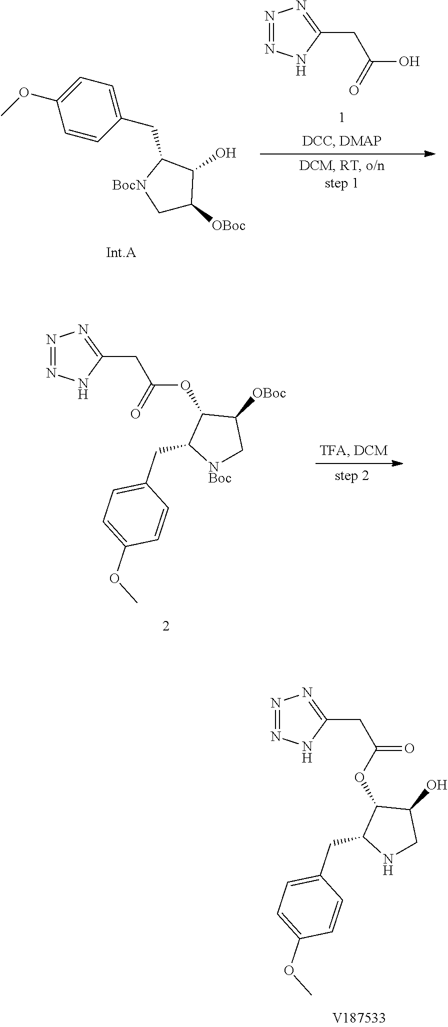

Step 1: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.24 mmol, 1.0 eq.) and 2H-1,2,3,4-tetrazol-5-ylacetic acid (30.3 mg, 0.24 mmol, 1.0 eq.) in DCM (5 mL) were added DCC (146.3 mg, 0.71 mmol, 3.0 eq.) and DMAP (43.3 mg, 0.35 mmol, 1.5 eq.) at 0° C. under nitrogen atmosphere. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl) oxy]-2-[(4-methoxyphenyl)methyl]-3-{[2-(2H−1,2,3,4-tetrazol-5-yl)acetyl]oxy}pyrrolidine-1-carboxylate (40 mg, 31.75%) as a white solid. MS: m/z: Calc'd for C₂₅H₃₅N₅O₈ [M+H−56]⁺478, found 478.

Step 2: To a stirred mixture of tert-butyl (2R,3S,4S)-4-[(tert-butoxycarbonyl)oxy]-2-[(4-methoxy phenyl)methyl]-3-{[2-(1H−1,2,3,4-tetrazol-5-yl)acetyl]oxy}pyrrolidine-1-carboxylate (30 mg, 0.06 mmol, 1.0 eq.) in DCM (2 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl 2-(1H−1,2,3,4-tetrazol-5-yl)acetate; trifluoroacetic acid (12.4 mg, 47.7%) as a white solid. MS: m/z: Calc'd for C₁₅H₁₉N₅O₄ [M+H]⁺334, found 334. ¹H NMR (400 MHz, Methanol-d₄) δ 7.19 (d, J=8.1 Hz, 2H), 6.96-6.89 (m, 2H), 5.14 (d, J=3.3 Hz, 1H), 4.43 (d, J=4.2 Hz, 1H), 4.31-4.18 (m, 2H), 3.79 (d, J=1.9 Hz, 3H), 3.60 (dd, J=12.7, 4.4 Hz, 1H), 3.24 (d, J=12.7 Hz, 1H), 3.05 (dd, J=14.4, 6.9 Hz, 1H), 2.94 (dd, J=14.3, 8.9 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 22% B in 10 min; Wave Length: 254 nm/220 nm nm; RT1(min): 8.68

### Synthetic Procedure of V187694

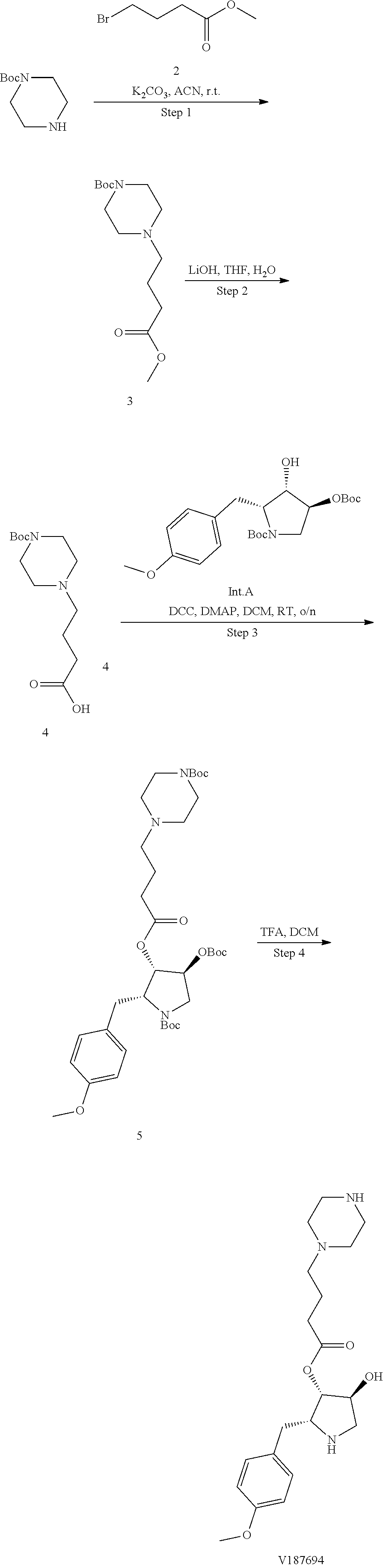

Step 1: To a stirred solution of tert-butyl piperazine-1-carboxylate (300 mg, 1.61 mmol, 1.0 eq.) and methyl 4-bromobutanoate (583.2 mg, 3.22 mmol, 2.0 eq.) in ACN (10 mL) was added K₂CO₃ (0.7 g, 4.83 mmol, 3.0 eq.) at room temperature. The resulting reaction mixture was stirred at ambient temperature for overnight. The mixture was filtered, the filter cake was washed with MeCN (1×1 mL). The filtrate was concentrated under reduced pressure. The residue was purified by silica gel column chromatography, eluted with PE/EA (1:1) to afford tert-butyl 4-(4-methoxy-4-oxobutyl)piperazine-1-carboxylate (400 mg, 86.7%) as a light yellow oil. MS: m/z: Calc'd for C₁₄H₂₆N₂O₄ [M+H]⁺287, found 287.

Step 2: To a stirred solution of tert-butyl 4-(4-methoxy-4-oxobutyl)piperazine-1-carboxylate (300 mg, 1.05 mmol, 1 eq.) in THF (10 mL) and H₂O (1 mL) was added LiOH (75.27 mg, 3.144 mmol, 3 eq.) at room temperature. The resulting reaction mixture was stirred at ambient temperature for overnight. The reaction mixture was concentrated and pH adjusted to˜3-4 with citric acid. The resulting mixture was extracted with EA. The combined organic layers were dried over sodium sulfate, concentrated in vacuo. The residue (300 mg) was purified by Prep-HPLC with the following conditions (0.05% TFA in H₂O/ACN) to afford 4-[4-(tert-butoxycarbonyl)piperazin-1-yl]butanoic acid (200 mg, 70.10%) as a colorless oil. MS: m/z: Calc'd for C₁₃H₂₄N₂O₄ [M+H]⁺273, found 273.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 100 mg, 0.24 mmol, 1.0 eq.) and 4-[4-(tert-butoxycarbonyl)piperazin-1-yl]butanoic acid (128.6 mg, 0.47 mmol, 2.0 eq.) in DCM (5 mL) were added DCC (73.1 mg, 0.35 mmol, 1.5 eq.) and DMAP (28.8 mg, 0.24 mmol, 1.0 eq.) at 0° C. under nitrogen atmosphere. The resulting reaction mixture was stirred at ambient temperature for overnight. Upon completion, the reaction mixture was concentrated in vacuo. The resulting residue was dissolved in DMSO and purified by reversed-phase column chromatography (0.05% TFA in water and acetonitrile) to afford tert-butyl 4-(4-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-4-oxobutyl)piperazine-1-carboxylate (150 mg, 93.7%) MS: m/z: Calc'd for C₃₅H₅₅N₃O₁₀ [M+H]⁺678, found 678.

Step 4: To a stirred mixture of tert-butyl 4-(4-{[(2R,3S,4S)-1-(tert-butoxycarbonyl)-4-[(tert-butoxycarbonyl)oxy]-2-[(4-methoxyphenyl)methyl]pyrrolidin-3-yl]oxy}-4-oxobutyl) piperazine-1-carboxylate (100 mg, 0.15 mmol, 1.0 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl 4-(piperazin-1-yl)butanoate (16.8 mg, 28.9%) as a white solid. MS: m/z: Calc'd for C₂₀H₃₁N₃O₄ [M+H]⁺378, found 378. ¹H NMR (400 MHz, DMSO-d₆) δ 7.12-7.05 (m, 2H), 6.86-6.78 (m, 2H), 5.10-5.05 (m, 1H), 4.66 (dd, J=4.0, 1.4 Hz, 1H), 3.93-3.91 (m, 1H), 3.71 (s, 3H), 3.16 (dd, J=11.6, 5.8 Hz, 2H), 2.69-2.65 (m, 4H), 2.62-2.52 (m, 6H), 2.41-2.33 (m, 2H), 2.29-2.19 (m, 5H), 1.70-1.68 (m, 2H). Prep-HPLC-conditions: Column: XBridge Prep Shield RP18 OBD C18 Column, 30*150 mm, 5 μm Water(10 nmol/L NH₄HCO₃) ACN 60 mL/min 2% B to 29% B in 10 min 9.65; Wave Length: 254 nm/220 nm nm; RT1(min): 9.65

### Synthetic Procedure of V186765

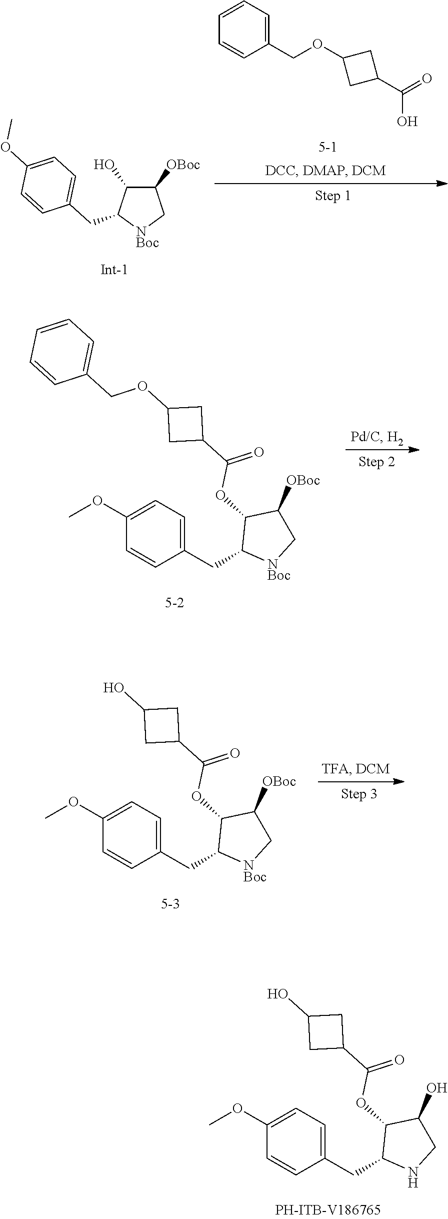

Step 1: The compound was synthesized according to Condensation Reaction with DCC and DMAP; General Procedure I using 3-(benzyloxy)cyclobutane-1-carboxylic acid (5-1, 97.4 mg, 0.47 mmol). The crude product was purified by reversed-phase column (0.05% NH₄HCO₃ in water and acetonitrile) to obtain product as a brown solid; yield: 120 mg, (83.1%). MS: m/z Calc'd for C₃₄H₄₅NO₉ [M+H]⁺613, found 613.

Step 2: Under a nitrogen atmosphere, Pd/C (80 mg) was added to a solution of tert-butyl (2R,3S,4S)-3-[3-(benzyloxy)cyclobutanecarbonyloxy]-4-[(tert-butoxycarbonyl)oxy]-2-[(4-methoxyphenyl)methyl]pyrrolidine-1-carboxylate (5-2, 170 mg, 0.278 mmol) in EA (10 mL), H₂ was subsequently introduced into the reaction system, and the resulting mixture was stirred at r.t. for 12 h. Upon completion, the reaction mixture was filtered and concentrated in vacuo to afford crude product which was used directly in the next step without further purification. MS: m/z: Calc'd for C₂₇H₃₉NO₉ [M+H]⁺522, found 522.

Step 3: The compound was synthesized according to Boc Deprotection; General Procedure III using tert-butyl (2R,3S,4S)-4-[(tert-butoxycarbonyl)oxy]-3-(3-hydroxycyclobutane carbonyloxy)-2-[(4-methoxyphenyl)methyl]pyrrolidine-1-carboxylate (5-3, 80 mg, 0.153 mmol). The crude product was purified by Prep-HPLC to afford a white solid; yield: 26.9 mg (51.9%). MS: m/z: Calc'd for C₁₇H₂₃NO₅ [M+H]⁺322; Found, 322. ¹H NMR (400 MHz, DMSO-d₆) δ 7.19 (m, 2H), 6.94-6.86 (m, 2H), 4.94-4.84 (m, 1H), 4.20-4.18 (m, 1H), 4.02-4.00 (m, 1H), 3.74 (s, 2H), 3.44-3.42 (m, 3H), 3.42-3.40 (m, 1H), 3.08 (d, J=12.8 Hz, 1H), 3.01-2.84 (m, 2H), 2.68-2.66 (m, 1H), 2.59-2.51 (m, 1H), 2.50-2.38 (m, 1H), 2.04-2.02 (m, 2H). Prep-HPLC purification conditions: XSelect CSH Prep C18 OBD Column, 5 μm, 30*150 mm; Mobile Phase A: Water (0.05% TFA), Mobile Phase B: ACN; Flow rate:60 mL/min; Gradient: 38% B to 50% B in 20 min; 254/220 nm; RT1:10.48; RT2:; Injection Volumn: ml; Number Of Runs:;

### Synthetic Procedure of V186777

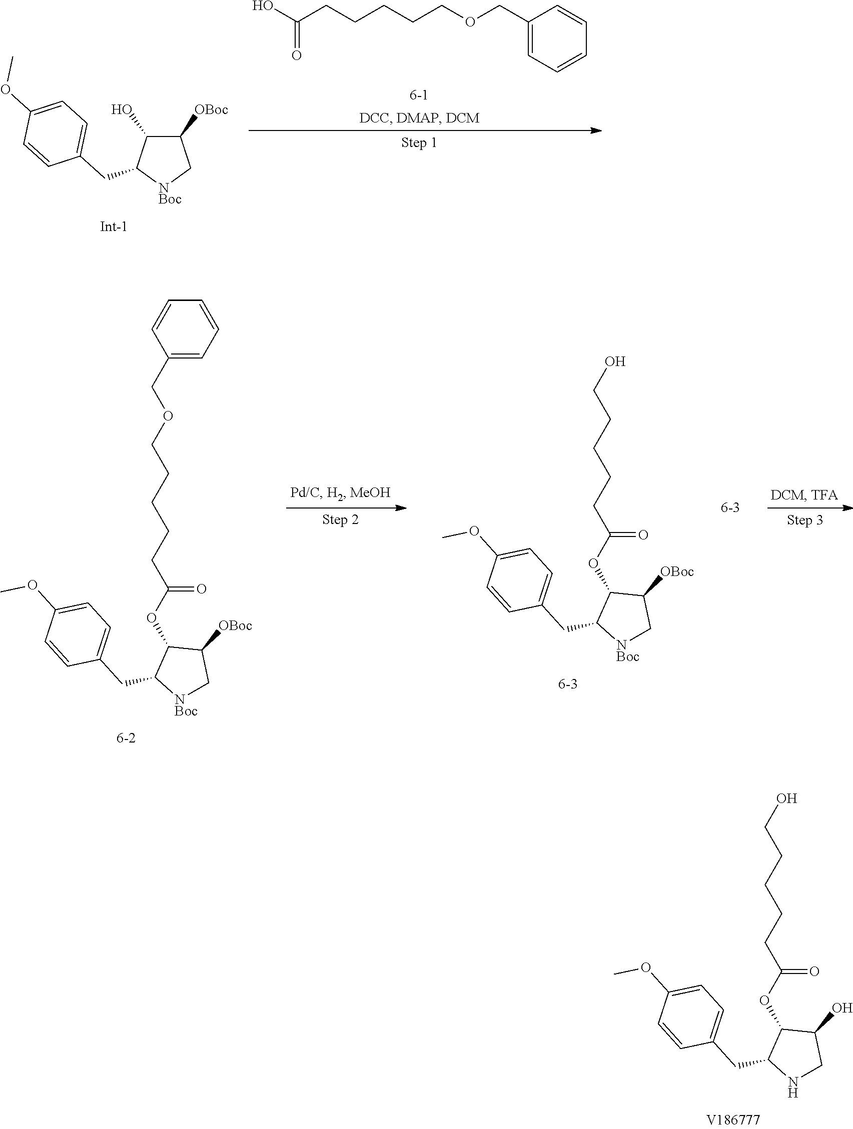

Step 1: The compound was synthesized according to Condensation Reaction with DCC and DMAP; General Procedure I using 6-(benzyloxy)hexanoic acid (6-1, 105.0 mg, 0.47 mmol). The crude product was purified by reversed-phase column (0.05% NH₄HCO₃ in water and acetonitrile) to obtain product as a brown solid; yield: 70 mg, (47.2%). MS: m/z Calc'd for C₃₅H₄₉NO₉ [M+H−100-56]⁺472, found 472.

Step 2: Under a nitrogen atmosphere, Pd/C (20 mg, 0.19 mmol, 1.2 eq.) was added to a solution of tert-butyl (2R,3S,4S)-3-{[6-(benzyloxy)hexanoyl]oxy}-4-[(tert-butoxycarbonyl)oxy]-2-[(4-methoxyphenyl)methyl]pyrrolidine-1-carboxylate (6-2, 100 mg, 0.16 mmol, 1 eq.) in MeOH (5 mL), H₂ was subsequently introduced into the reaction system, and the resulting mixture was stirred at r.t. for 2 h. The reaction mixture was filtered to obtain tert-butyl (2R,3S,4S)-4-[(tert-butoxycarbonyl)oxy]-3-[(6-hydroxyhexanoyl)oxy]-2-[(4-methoxyphenyl)methyl]pyrrolidine-1-carboxylate (60 mg, 70.1%) as a white solid. MS: m/z: Calc'd for C₂₈H₄₃NO₉ [M+H−100−56]⁺382, found 382.

Step 3: The compound was synthesized according to Boc Deprotection; General Procedure III using tert-butyl (2R,3S,4S)-4-[(tert-butoxycarbonyl)oxy]-3-[(6-hydroxyhexanoyl)oxy]-2-[(4-methoxyphenyl)methyl]pyrrolidine-1-carboxylate (6-3, 60 mg, 0.11 mmol). The crude product was purified by Prep-HPLC to afford a yellow semi-solid; yield: 18.2 mg (99.6%). MS: m/z: Calc'd for C₁₈H₂₇NO₅ [M+H]⁺338, found 338. ¹H NMR (400 MHz, Methanol-d₄) δ 7.27-7.19 (m, 2H), 6.98-6.90 (m, 2H), 5.13-5.08 (m, 1H), 4.38-4.33 (m, 1H), 4.24-4.14 (m, 1H), 3.80 (s, 3H), 3.63-3.54 (m, 3H), 3.21 (d, J=12.7 Hz, 1H), 3.09 (dd, J=14.3, 6.8 Hz, 1H), 2.95 (dd, J=14.3, 8.8 Hz, 1H), 2.58-2.47 (m, 2H), 1.80-1.67 (m, 2H), 1.64-1.53 (m, 2H), 1.52-1.39 (m, 2H). Prep-HPLC purification conditions: Column: XselectCSH Prep OBD C18 Column, 30*150 mm, 5 μm; Mobile Phase A: Water (0.05% TFA) Mobile Phase B: ACN; Flow rate: 60 mL/min; Gradient: 24% B to 54% B in 10 min; Wave Length: 254 nm/220 nm nm; RT1(min): 8.68.

### Synthetic Procedure of V186779

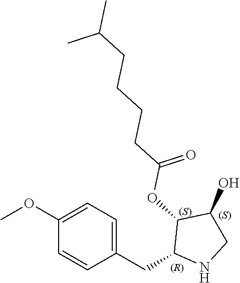

The title compound was prepared in 23.7% overall yield as a white solid according to Condensation Reaction with DCC and DMAP; General Procedure I using decanoic C₈ acid in STEP 1; Boc Deprotection; General Procedure III in STEP 2. MS: m/z Calc'd for C₂₀H₃₁N₀₄, [M+H]⁺350, found 350. 1H NMR (400 MHz, Methanol-d4) δ 7.31-7.11 (m, 2H), 6.99-6.78 (m, 2H), 5.11-5.08 (m, 1H), 4.41-4.29 (m, 1H), 4.19-4.17 (m, 1H), 3.80 (s, 3H), 3.58 (dd, J=12.7, 4.4 Hz, 1H), 3.21 (d, J=12.7 Hz, 1H), 3.09 (dd, J=14.2, 6.8 Hz, 1H), 2.95 (dd, J=14.3, 8.8 Hz, 1H), 2.50-2.48 (m, 2H), 1.68-1.66 (m, 2H), 1.58-1.56 (m, 1H), 1.45-1.35 (m, 2H), 1.32-1.21 (m, 2H), 0.92 (d, J=6.6 Hz, 6H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 10% B to 40% B in 10 min; Wave Length: 254 nm/220 nm nm; RT1(min): 8.68.

### Synthetic Procedure of V186780

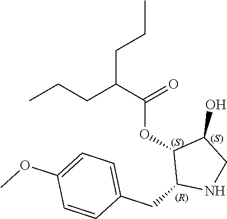

The title compound was prepared in 24.3% overall yield as a white solid according to Condensation Reaction with DCC and DMAP; General Procedure I using valproic acid in STEP 1; Boc Deprotection; General Procedure III in STEP 2. MS: m/z Calc'd for C₂₀H₃₁NO₄, [M+H]⁺350, found 350. ¹H NMR (400 MHz, Methanol-d₄) δ 7.31-7.11 (m, 2H), 6.99-6.78 (m, 2H), 5.11-5.09 (m, 1H), 4.41-4.29 (m, 1H), 4.19-4.17 (m, 1H), 3.80 (s, 3H), 3.58 (dd, J=12.7, 4.4 Hz, 1H), 3.21 (d, J=12.7 Hz, 1H), 3.09 (dd, J=14.2, 6.8 Hz, 1H), 2.95 (dd, J=14.3, 8.8 Hz, 1H), 2.50-2.48 (m, 2H), 1.68-1.66 (m, 2H), 1.58-1.56 (m, 1H), 1.45-1.35 (m, 2H), 1.32-1.21 (m, 2H), 0.92 (d, J=6.6 Hz, 6H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 10% B to 40% B in 10 min; Wave Length: 254 nm/220 nm nm; RT1(min): 8.68.

### Synthetic Procedure of V186783

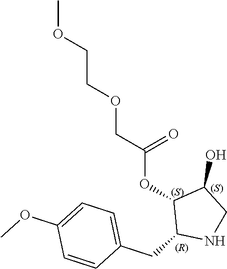

The title compound was prepared in 24.4% overall yield as a white solid according to Condensation Reaction with DCC and DMAP; General Procedure I using 2-(2-methoxyethoxy) acetic acid in STEP 1; Boc Deprotection; General Procedure III in STEP 2. MS: m/z Calc'd for C₂₀H₃₁NO₄, [M+H]⁺340, found 340. ¹H NMR (400 MHz, Methanol-d₄) δ 7.31-7.11 (m, 2H), 6.99-6.78 (m, 2H), 5.11-5.09 (m, 1H), 4.41-4.29 (m, 1H), 4.19-4.17 (m, 1H), 3.80 (s, 3H), 3.58 (dd, J=12.7, 4.4 Hz, 1H), 3.21 (d, J=12.7 Hz, 1H), 3.09 (dd, J=14.2, 6.8 Hz, 1H), 2.95 (dd, J=14.3, 8.8 Hz, 1H), 2.50-2.48 (m, 2H), 1.68-1.66 (m, 2H), 1.58-1.56 (m, 1H), 1.45-1.35 (m, 2H), 1.32-1.21 (m, 2H), 0.92 (d, J=6.6 Hz, 6H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 26% B in 10 min; Wave Length: 254 nm/220 nm nm; RT1(min): 8.68.

### Synthetic Procedure of V186811

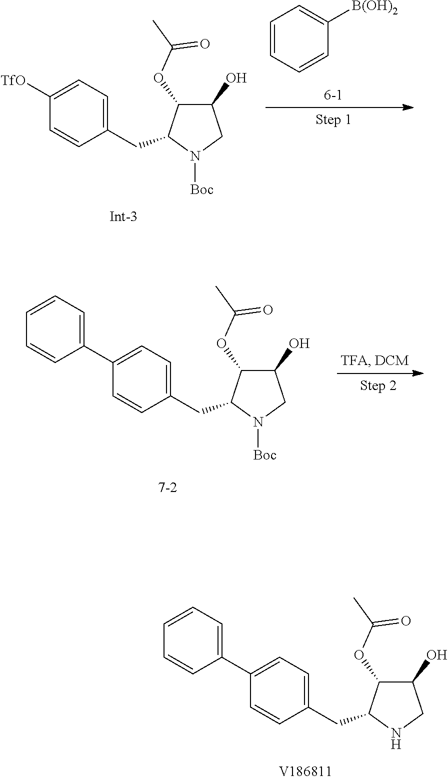

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (Int-3, 150 mg, 0.31 mmol, 1 eq.) and phenyl boronic acid (75.7 mg, 0.62 mmol, 2 eq.) in 1,4-dioxane (5 mL) were added LiCl (39.5 mg, 0.93 mmol, 3 eq.) and Pd(PPh₃)₄ (35.9 mg, 0.03 mmol, 0.1 eq.) at room temperature. The reaction was placed under vacuum, sonicated and backfilled with nitrogen. The resulting mixture was stirred at 90° C. for 12 h. After completion of reaction monitored by LCMS. The reaction mixture was quenched with water and extracted with EA. The extracts were washed with brine, dried over anhydrous sodium sulfate, filtered and concentrated. The residue was purified by reversed-phase flash to obtain tert-butyl (2R,3S,4S)-3-(acetyloxy)-2-{[1,1′-biphenyl]-4-ylmethyl}-4-hydroxypyrrolidine-1-carboxylate (40 mg, 31.3%) as a light yellow oil. MS: m/z: Calc'd for C₂₄H₂₉NO₅[M+H]⁺412, found 412.

Step 2: The compound was synthesized according to Boc Deprotection; General Procedure III using tert-butyl (2R,3S,4S)-3-(acetyloxy)-2-{[1,1′-biphenyl]-4-ylmethyl}-4-hydroxypyrrolidine-1-carboxylate (7-2, 40 mg, 0.10 mmol). The crude product was purified by Prep-HPLC to afford a yellow semi-solid; yield: 26.1 mg (63.0%). MS: m/z: Calc'd for C₁₉H₂₁NO₃ [M+H]⁺312, found 312.

¹H NMR (400 MHz, Methanol-d₄) δ 7.69-7.59 (m, 4H), 7.50-7.33 (m, 5H), 5.15 (d, J=3.4 Hz, 1H), 4.40 (d, J=4.4 Hz, 1H), 4.33-4.23 (m, 1H), 3.63 (dd, J=12.7, 4.4 Hz, 1H), 3.23 (dd, J=13.7, 6.8 Hz, 2H), 3.08 (dd, J=14.3, 8.9 Hz, 1H), 2.22 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 6% B to 36% B in 10 min; Wave Length: 254 nm/220 nm nm; RT1(min): 8.68

### Synthetic Procedure of V186940

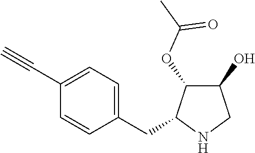 [SMILES: C#Cc1ccc(C[C@H]2NC[C@H](O)[C@H]2OC(C)=O)cc1]

The title compound was prepared in 47.2% yield as a white solid according to Boc Deprotection; General Procedure III using Int-4 in STEP 1. MS: m/z Calc'd for C₁₅H₁₇NO₃ [M+H]⁺260, found 260. ¹H NMR (400 MHz, Methanol-d₄) δ 7.49 (d, J=8.2 Hz, 2H), 7.32 (d, J=8.1 Hz, 2H), 5.11 (d, J=3.4 Hz, 1H), 4.38 (d, J=4.4 Hz, 1H), 4.22 (s, 1H), 3.60 (dd, J=12.6, 4.5 Hz, 1H), 3.53 (s, 1H), 3.19 (dd, J=13.5, 7.7 Hz, 2H), 3.03 (dd, J=14.3, 9.0 Hz, 1H), 2.19 (s, 3H). Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 29% B in 10 min; Wave Length: 254 nm/220 nm nm; RT1(min): 8.68.

### Synthetic Procedure of V187172

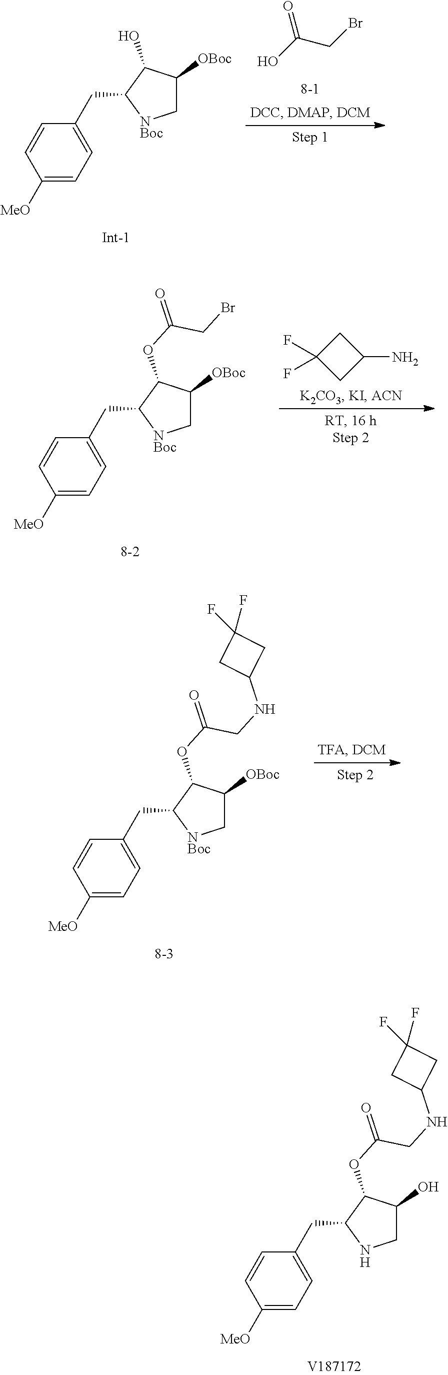

Step 1: The compound was synthesized according to Condensation Reaction with DCC and DMAP; General Procedure I using 2-bromoacetic acid (8-1, 131.2 mg, 0.94 mmol). The crude product was purified by reversed-phase column (0.05% NH₄HCO₃ in water and acetonitrile) to obtain product as a yellow oil; yield: 180 mg, (70%). MS: m/z Calc'd for C₂₄H₃₄BrNO₈ [M+H]⁺544, found 544.

Step 2: To a solution of 3,3-difluorocyclobutan-1-amine (17 mg, 0.15 mmol, 1 eq.), tert-butyl (2R,3S,4S)-4-[(tert-butoxycarbonyl)oxy]-3-hydroxy-2-[(4-methoxyphenyl)methyl]pyrrolidine-1-carboxylate (8-2, 67.2 mg, 0.15 mmol, 1 eq.) in ACN (4 mL) was added K₂CO₃ (65.8 mg, 0.47 mmol, 3 eq.) and KI (26.3 mg, 0.15 mmol, 1 eq.) at room temperature. The resulting mixture was stirred at room temperature for overnight. Desired product could be detected by LCMS. The resulting mixture was filtered, the filter cake was washed with ACN (40 mL) (2×20 mL). The filtrate was concentrated under reduced pressure. The residue was purified by reversed-phase flash chromatography to afford tert-butyl (2R,3S,4S)-4-[(tert-butoxycarbonyl)oxy]-3-({2-[(3,3-difluorocyclobutyl)amino]acetyl}oxy)-2-[(4-methoxy phenyl)methyl]pyrrolidine-1-carboxylate (56 mg, 54.5%) as a yellow oil. MS: m/z: Calc'd for C₂₈H₄₀F₂N₂O₈ [M+H]⁺571, found 571.

Step 3: The compound was synthesized according to Boc Deprotection; General Procedure III using tert-butyl (2R,3S,4S)-4-[(tert-butoxycarbonyl)oxy]-3-({2-[(3,3-difluorocyclo butyl)amino]acetyl}oxy)-2-[(4-methoxyphenyl)methyl]pyrrolidine-1-carboxylate (8-3, 46 mg, 0.08 mmol). The crude product was purified by Prep-HPLC to afford a yellow semi-solid; yield: 22.9 mg (58.3%). MS: m/z: Calc'd for C₁₈H₂₄F₂N₂O₄[M+H]⁺371, found 371. ¹H NMR (400 MHz, Methanol-d₄) δ 7.31-7.22 (m, 2H), 6.98-6.90 (m, 2H), 5.25 (d, J=3.5 Hz, 1H), 4.48-4.42 (m, 1H), 4.33-4.05 (m, 3H), 3.83 (t, J=7.5 Hz, 1H), 3.67 (dd, J=12.8, 4.4 Hz, 1H), 3.32-3.19 (m, 3H), 3.24 (d, J=12.8 Hz, 1H), 3.18-2.79 (m, 6H). Prep-HPLC purification conditions: Column: Xselect CSH Prep C18 Column, 30*150 mm, 5 μm; Mobile Phase A: Water (0.1% FA), Mobile Phase B: ACN; Flow rate: 60 mL/min; Gradient: 2% B to 21% B in 10 min; Wave Length: 254 nm/220 nm nm; RT1(min): 8.68.

### Synthetic Procedure of V187689

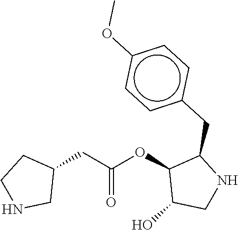

The title compound was prepared in 95.1% overall yield as a white solid according to Condensation Reaction with DCC and DMAP; General Procedure I using (R)-2-(1-(tert-butoxycarbonyl)pyrrolidin-3-yl)acetic acid in STEP 1; Boc Deprotection; General Procedure III using HCl (4 M in dioxane) instead of TFA in STEP 2. MS: m/z Calc'd for C₁₈H₂₆N₂O₄, [M+H]⁺335, found 335. 1H NMR (400 MHz, Methanol-d4) δ 8.55 (s, 2H), 7.29-7.21 (m, 2H), 6.97-6.90 (m, 2H), 5.10 (d, J=3.5 Hz, 1H), 4.35 (d, J=4.4 Hz, 1H), 4.20-4.11 (m, 1H), 3.80 (s, 3H), 3.54-3.66 (m, 2H), 3.37-3.48 (m, 1H), 3.33-3.23 (m, 1H), 3.19 (d, J=12.7 Hz, 1H), 3.12-2.94 (m, 3H), 2.75 (d, J=2.9 Hz, 3H), 2.36-2.24 (m, 1H), 1.82-1.68 (m, 1H). Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm nm; RT1(min): 12.72.

### Synthetic Procedure of V187690

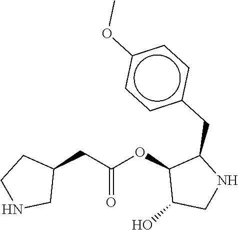

The title compound was prepared in 68.5% overall yield as a white solid according to Condensation Reaction with DCC and DMAP; General Procedure I using (S)-2-(1-(tert-butoxycarbonyl)pyrrolidin-3-yl)acetic acid in STEP 1; Boc Deprotection; General Procedure III using HCl (4 M in dioxane) instead of TFA in STEP 2. MS: m/z Calc'd for C₁₈H₂₆N₂O₄ [M+H]⁺335, found 335. 1H NMR (400 MHz, Methanol-d4) δ 8.55 (s, 2H), 7.27-7.19 (m, 2H), 6.97-6.89 (m, 2H), 5.09 (d, J=3.4 Hz, 1H), 4.35 (d, J=4.4 Hz, 1H), 4.18-4.09 (m, 1H), 3.80 (s, 3H), 3.65-3.53 (m, 2H), 3.49-3.38 (m, 1H), 3.33-3.23 (m, 1H), 3.17 (d, J=12.7 Hz, 1H), 3.11-2.93 (m, 3H), 2.83-2.64 (m, 3H), 2.35-2.25 (m, 1H), 1.81-1.67 (m, 1H). Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm nm; RT1(min): 13.34.

### Synthetic Procedure of V187720

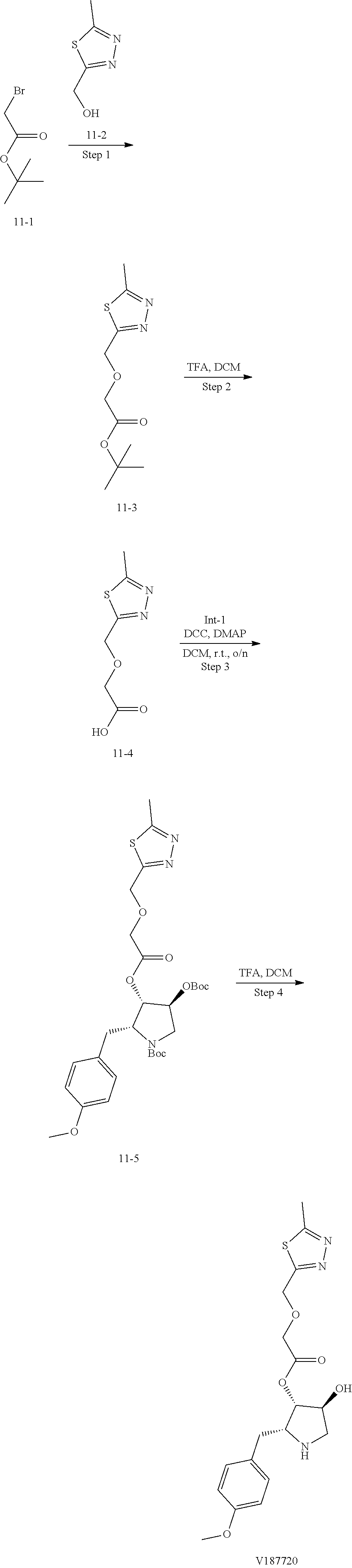

Step 1: To a stirred solution of (5-methyl-1,3,4-thiadiazol-2-yl)methanol (11-2, 250 mg, 1.92 mmol, 1.0 eq.) in THF (5 mL) were added NaH (153.8 mg, 3.84 mmol, 2.0 eq.) and tert-butyl 2-bromoacetate (11-1, 748.8 mg, 3.84 mmol, 2.0 eq.) at 0° C. The reaction was stirred at room temperature for overnight. After completion of the reaction monitored by LCMS, the resulting mixture was concentrated under reduced pressure. The residue product was purified by reversed phase flash to afford tert-butyl 2-[(5-methyl-1,3,4-thiadiazol-2-yl)methoxy]acetate (330 mg, 70.5%) as a white solid. MS: m/z: Calc'd for C₁₀H₁₆N₂O₃S, [M+H]⁺245; Found 245.

Step 2: To a stirred solution of tert-butyl 2-[(5-methyl-1,3,4-thiadiazol-2-yl)methoxy]acetate (11-3, 160 mg, 0.65 mmol, 1.0 eq.) in DCM (1 mL) was added TFA (0.2 mL) at 0° C. The reaction was stirred at room temperature for 2 h. After completion of the reaction monitored by LCMS, the mixture was concentrated under reduced pressure to afford crude product which was used directly in the next step without further purification. MS: m/z: Calc'd for C₆H₈N₂₀₃S [M+H]⁺189, Found 189.

Step 3: The compound was synthesized according to Condensation Reaction with DCC and DMAP; General Procedure I using [(5-methyl-1,3,4-thiadiazol-2-yl)methoxy]acetic acid (11-4, 79.9 mg, 0.42 mmol). The crude product was purified by reversed-phase column (0.05% NH₄HCO₃ in water and acetonitrile) to obtain product as a yellow oil; yield: 90 mg, (71.3%). MS: m/z Calc'd for C₂₈H₃₉N₃₀₉S [M+H]⁺594, Found 594.

Step 4: The compound was synthesized according to Boc Deprotection; General Procedure III using tert-butyl (2R,3S,4S)-4-[(tert-butoxycarbonyl)oxy]-2-[(4-methoxyphenyl)methyl]-3-({2-[(5-methyl-1,3,4-thiadiazol-2-yl)methoxy]acetyl}oxy)pyrrolidine-1-carboxylate (11-5, 75 mg, 0.12 mmol). The crude product was purified by Prep-HPLC to afford a yellow semi-solid; yield: 28.9 mg (44.9%). MS: m/z: Calc'd for C₁₈H₂₃N₃O₅S [M+H]⁺394, Found 394. 1H NMR (400 MHz, Methanol-d4) δ 7.28-7.20 (m, 2H), 6.99-6.91 (m, 2H), 5.20-5.15 (m, 1H), 5.06 (s, 2H), 4.48-4.37 (m, 2H), 4.24-4.22 (m, 1H), 3.80 (s, 3H), 3.61-3.59 (m, 1H), 3.24 (d, J=12.7 Hz, 1H), 3.10-3.08 (m, 1H), 2.97-2.95 (m, 1H), 2.80 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.68.

### Synthetic Procedure of V187731

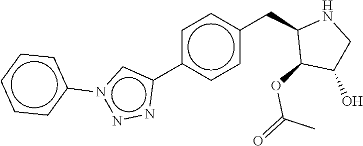

The title compound was prepared in 23.6% overall yield as white solid according to Cyclization reaction for synthesis of Substituted triazoles; General Procedure VI using phenyl azide in STEP 1; Boc Deprotection; General Procedure III in STEP 2. MS: m/z Calc'd for C₂₁H₂₂N₄O₃ [M+H]⁺379, found 379. 1H NMR (400 MHz, Methanol-d4) δ 8.95 (s, 1H), 8.01-7.90 (m, 4H), 7.64-7.62 (m, 2H), 7.59-7.50 (m, 1H), 7.50-7.44 (m, 2H), 5.15 (m, 1H), 4.40 (s, 1H), 4.29-4.27 (m, 1H), 3.64 (s, 1H), 3.29-3.19 (m, 2H), 3.10-3.08 (m, 1H), 2.23 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 10% B to 35% B in 10 min; Wave Length: 254 nm/220 nm; RT1(min): 8.68.

### Synthetic Procedure of V187750

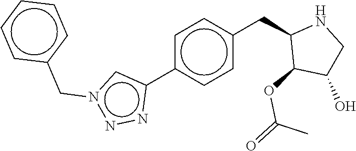

The title compound was prepared in 25.9% overall yield as white solid according to Cyclization reaction for synthesis of Substituted triazoles; General Procedure VI using (azidomethyl)benzene in STEP 1; Boc Deprotection; General Procedure III in STEP 2. MS: m/z Calc'd for C₂₂H₂₄N₄O₃ [M+H]⁺393, found 393. 1H NMR (400 MHz, Methanol-d4) δ 8.34 (s, 1H), 7.88-7.81 (m, 2H), 7.46-7.33 (m, 7H), 5.66 (s, 2H), 5.16-5.10 (m, 1H), 4.42-4.36 (m, 1H), 4.27-4.25 (m, 1H), 3.63-3.61 (m, 1H), 3.27-3.16 (m, 2H), 3.07-3.05 (m, 1H), 2.21 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 9% B to 34% B in 10 min; Wave Length: 254 nm/220 nm; RT1(min): 8.68.

### Synthetic Procedure of V187263

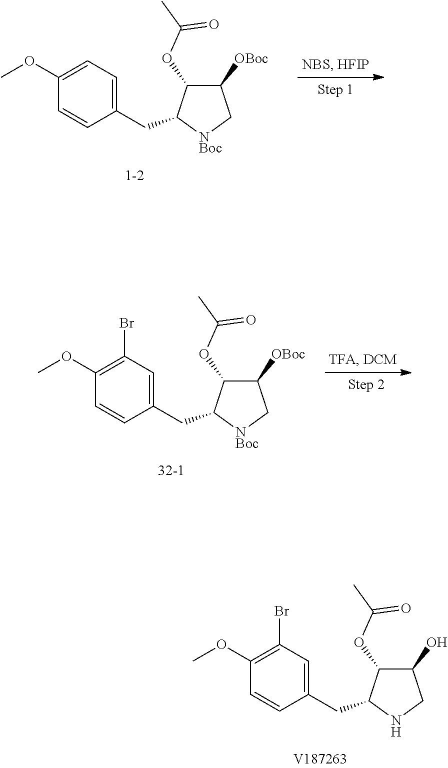

Step 1: To a stirred solution of tert-butyl (2R,3S,4S)-3-(acetyloxy)-4-[(tert-butoxycarbonyl)oxy]-2-[(4-methoxyphenyl)methyl]pyrrolidine-1-carboxylate (1-2, 430 mg, 0.92 mmol, 1.0 eq.) in 1,1,1,3,3,3-hexafluoropropan-2-ol (10 mL) was added NBS (180.8 mg, 1.02 mmol, 1.1 eq.) in portions at 0° C. The resulting mixture was stirred 15 min at 0° C. under a nitrogen atmosphere. Desired product could be detected by LCMS. The resulting mixture was concentrated under reduced pressure. The residue was purified by reversed-phase flash chromatography to afford tert-butyl (2R,3S,4S)-3-(acetyloxy)-2-[(3-bromo-4-methoxy phenyl)methyl]-4-[(tert-butoxycarbonyl)oxy]pyrrolidine-1-carboxylate (450 mg, 89.5%) as a light yellow oil. MS: m/z: Calc'd for C₂₄H₃₄BrNO₈ [M+H⁺22+41]+607; Found, 607.

Step 2: The title compound was prepared in 24.9% yield as a yellow semi-solid according to Boc Deprotection; General Procedure III in STEP 2. MS: m/z Calc'd for C₁₄H₁₈BrNO₄ [M+H]⁺344; Found, 344. 1H NMR (400 MHz, Methanol-d4) δ 7.55 (d, J=2.2 Hz, 1H), 7.29 (dd, J=8.4, 2.2 Hz, 1H), 7.05 (d, J=8.4 Hz, 1H), 5.09 (d, J=3.4 Hz, 1H), 4.38 (d, J=4.3 Hz, 1H), 4.22-4.18 (m, 1H), 3.89 (s, 3H), 3.63 (dd, J=12.7, 4.5 Hz, 1H), 3.23 (d, J=12.7 Hz, 1H), 3.10 (dd, J=14.3, 6.7 Hz, 1H), 2.99 (dd, J=14.3, 8.9 Hz, 1H), 2.20 (s, 3H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 5% B to 35% B in 10 min; Wave Length: 254 nm/220 nm nm; RT1(min): 9.37.

### Synthetic Procedure of V188046

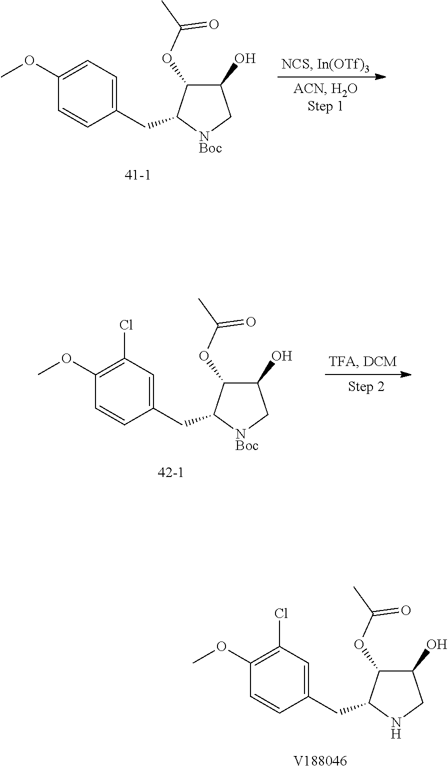 [SMILES: COc1ccc(C[C@@H]2[C@H](OC(C)=O)[C@@H](O)CN2C(=O)OC(C)(C)C)cc1 ; COc1ccc(C[C@@H]2[C@H](OC(C)=O)[C@@H](O)CN2C(=O)OC(C)(C)C)cc1Cl ; COc1ccc(C[C@H]2NC[C@H](O)[C@H]2OC(C)=O)cc1Cl]

Step 1: To a stirred solution of tert-butyl (2R,3S,4S)-3-(acetyloxy)-4-hydroxy-2-[(4-methoxyphenyl)methyl]pyrrolidine-1-carboxylate (41-1, 100 mg, 0.27 mmol, 1.0 eq.) in ACN (10 mL) and H₂O (10 mL) was added NCS (130.7 mg, 0.82 mmol, 3.0 eq.) and indium(iii) trifluoromethanesulfonate (30.8 mg, 0.06 mmol, 0.2 eq.) in portions at 0° C. The resulting mixture was stirred 48 h at 25° C. under a nitrogen atmosphere. Desired product could be detected by LCMS. The residue was purified by reversed-phase flash chromatography to afford tert-butyl (2R,3S,4S)-3-(acetyloxy)-2-[(3-chloro-4-methoxyphenyl)methyl]-4-hydroxypyrrolidine-1-carboxylate (70 mg, 64.0%) as a white solid. MS: m/z: Calc'd for C₁₉H₂₆C₁NO₆ [M+H−56]⁺344; Found, 344.

Step 2: The title compound was prepared in 24.1% yield as a light yellow semi-solid according to Boc Deprotection; General Procedure III in STEP 2. MS: m/z Calc'd for C₁₄H₁₈ClNO₄ [M+H]⁺300; Found, 300. 1H NMR (400 MHz, Methanol-d4) δ 7.38 (d, J=2.2 Hz, 1H), 7.24 (dd, J=8.4, 2.2 Hz, 1H), 7.09 (d, J=8.4 Hz, 1H), 5.11 (d, J=3.4 Hz, 1H), 4.38 (d, J=4.3 Hz, 1H), 4.22-4.18 (m, J=9.5, 6.5, 3.4 Hz, 1H), 3.90 (s, 3H), 3.61 (dd, J=12.7, 4.4 Hz, 1H), 3.23 (d, J=12.7 Hz, 1H), 3.10 (dd, J=14.4, 6.5 Hz, 1H), 2.97 (dd, J=14.4, 9.1 Hz, 1H), 2.20 (s, 3H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 9.07.

### Synthetic Procedure of V187751

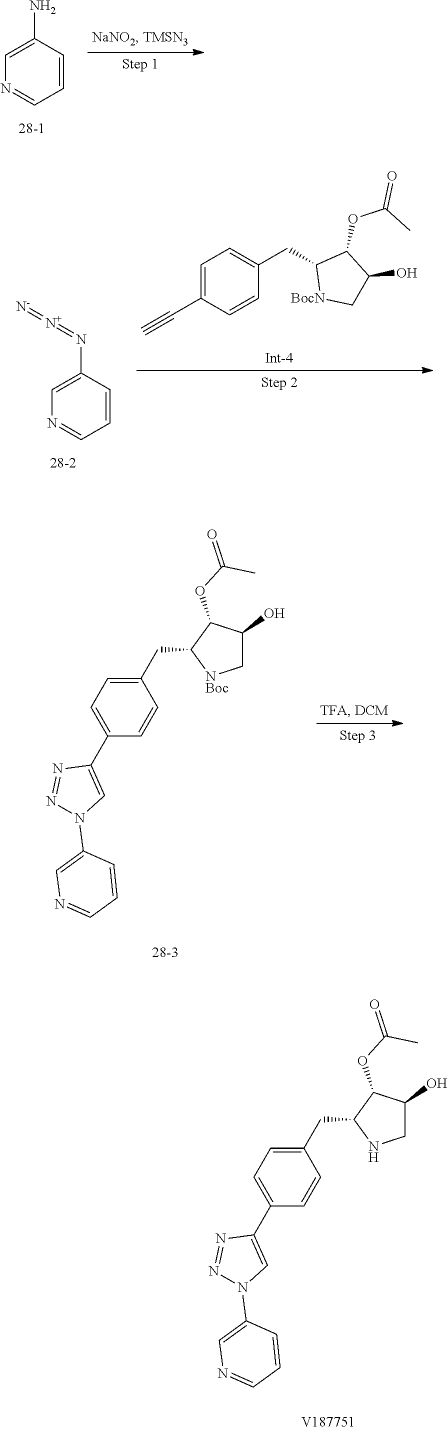 [SMILES: Nc1cccnc1 ; [N-]=[N+]=Nc1cccnc1 ; C#Cc1ccc(C[C@@H]2[C@H](OC(C)=O)[C@@H](O)CN2C(=O)OC(C)(C)C)cc1 ; CC(=O)O[C@H]1[C@@H](Cc2ccc(-c3cn(-c4cccnc4)nn3)cc2)N(C(=O)OC(C)(C)C)C[C@@H]1O ; CC(=O)O[C@@H]1[C@@H](O)CN[C@@H]1Cc1ccc(-c2cn(-c3cccnc3)nn2)cc1]

Step 1: To a stirred solution of 3-aminopyridine (28-1, 500 mg, 5.31 mmol, 1 eq.) in HCl (6M) (5.5 mL) was treated with NaNO₂ (549.8 mg, 7.97 mmol, 1.5 eq.) in water (13 mL) for 30 min at 0° C. under nitrogen atmosphere followed by the addition of NaN₃ (1.4 g, 21.23 mmol, 4.0 eq.) in water (1.4 ml) dropwise at 0° C. The resulting mixture was stirred at room temperature for 2 h under nitrogen atmosphere. LCMS showed the reaction was completed. The aqueous layer was extracted with EA (2×20 mL). After filtration, the filtrate was concentrated under reduced pressure. The crude product was used in the next step directly without further purification. MS: m/z: Calc'd for C₅H₄N₄ [M+H]⁺121, found 121.

Step 2 & 3: The title compound was prepared in 39.8% overall yield as a white solid according to Cyclization reaction for synthesis of Substituted triazoles; General Procedure VI using 3-azidopyridine (28-2) in STEP 2; Boc Deprotection; General Procedure III in STEP 3. MS: m/z Calc'd for C₂₀H₂₁N₅O₃ [M+H]⁺380, found 380. 1H NMR (400 MHz, Methanol-d4) δ 9.19 (s, 1H), 9.08 (d, J=1.5 Hz, 1H), 8.72 (d, J=4.8 Hz, 1H), 8.43 (dd, J=7.8, 2.1 Hz, 1H), 7.99 (dd, J=8.2, 1.6 Hz, 2H), 7.72 (dd, J=8.4, 4.8 Hz, 1H), 7.48 (d, J=7.9 Hz, 2H), 5.15 (s, 1H), 4.41 (d, J=4.3 Hz, 1H), 4.30 (s, 1H), 3.64 (dd, J=12.8, 4.3 Hz, 1H), 3.28-3.21 (m, 2H), 3.11 (dd, J=14.3, 9.0 Hz, 1H), 2.23 (d, J=1.4 Hz, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.68.

### Synthetic Procedure of V187840

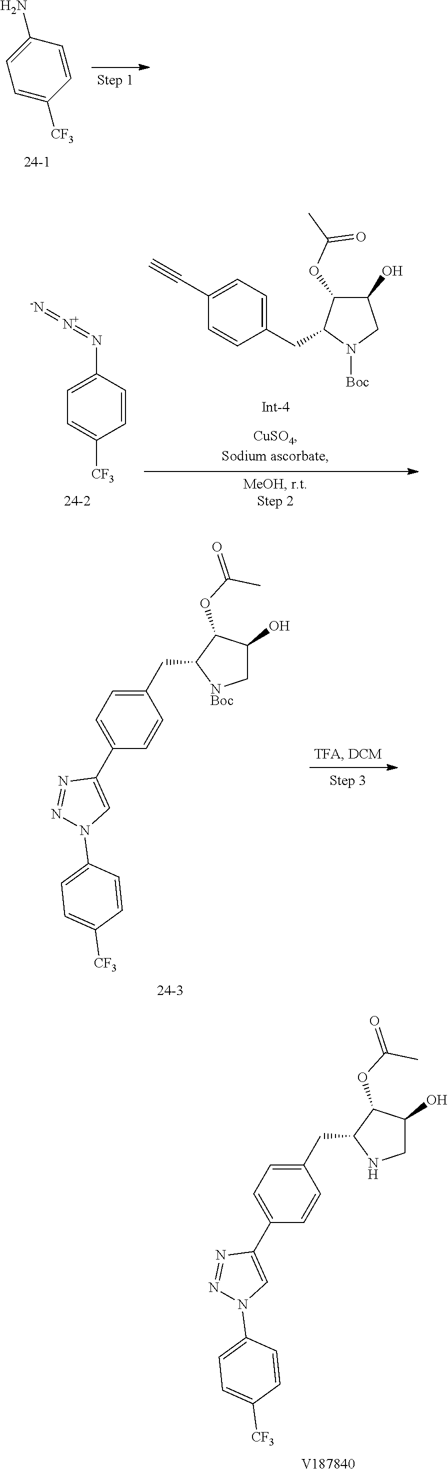

Step 1: To a stirred solution of P-trifluoromethylaniline (24-1, 100 mg, 0.62 mmol, 1 eq.) and Azidotrimethylsilane (107.2 mg, 0.93 mmol, 1.5 eq.) in ACN (3 mL) was added tert-butyl nitrite (96.0 mg, 0.93 mmol, 1.5 eq.) at 0° C. The resulting mixture was stirred for 1 h at room temperature. After completion of the reaction monitored by LCMS, the resulting mixture was concentrated under reduced pressure to afford 1-azido-4-(trifluoromethyl) benzene (100 mg, 86.1%) as a yellow oil which used directly in the next step without further purification. MS: m/z: Calc'd for C₇H₄F₃N₃ [M+H]⁺188; found 188.

Step 2 & 3: The title compound was prepared in 30.8% overall yield as white solid according to Cyclization reaction for synthesis of Substituted triazoles; General Procedure VI using 1-azido-4-(trifluoromethyl)benzene (24-2) in STEP 2; Boc Deprotection; General Procedure III in STEP 3. MS: m/z Calc'd for C₂₂H₂₁F₃N₄O₃ [M+H]⁺447, Found 447. 1H NMR (400 MHz, Methanol-d4) δ 9.09 (s, 1H), 8.19 (d, 2H), 7.98-7.96 (m, 4H), 7.48 (d, 2H), 5.15 (d, 1H), 4.41 (d, 1H), 4.31-4.29 (m, 1H), 3.65-3.63 (m, 1H), 3.25-3.23 (m, 2H), 3.12-3.10 (m, 1H), 2.23 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 17% B to 47% B in 10 min; Wave Length: 254 nm/220 nm nm; RT1(min): 8.2.

### Synthetic Procedure of V187855

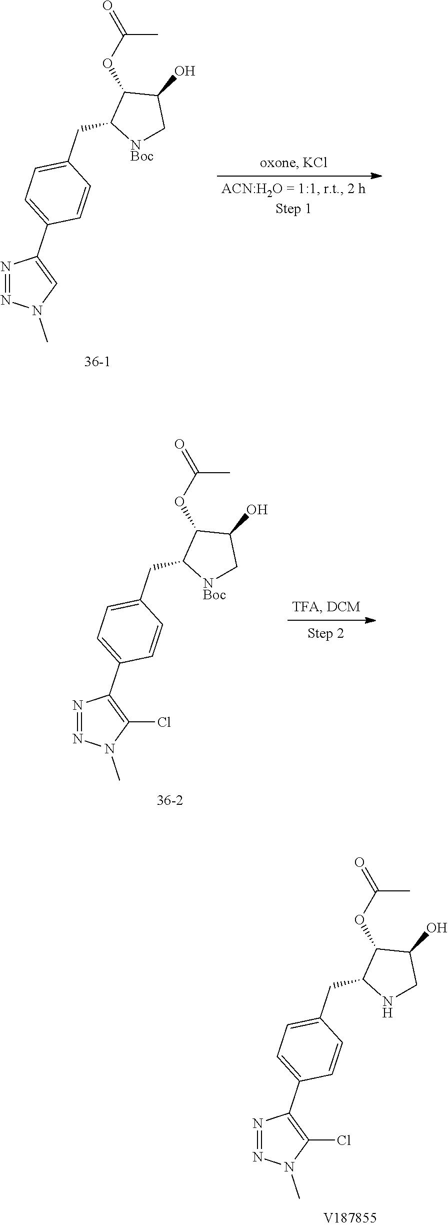 [SMILES: CC(=O)O[C@H]1[C@@H](Cc2ccc(-c3cn(C)nn3)cc2)N(C(=O)OC(C)(C)C)C[C@@H]1O ; CC(=O)O[C@H]1[C@@H](Cc2ccc(-c3nnn(C)c3Cl)cc2)N(C(=O)OC(C)(C)C)C[C@@H]1O ; CC(=O)O[C@@H]1[C@@H](O)CN[C@@H]1Cc1ccc(-c2nnn(C)c2Cl)cc1]

Step 1: To a stirred solution of tert-butyl (2R,3S,4S)-3-(acetyloxy)-4-hydroxy-2-{[4-(1-methyl-1,2,3-triazol-4-yl)phenyl]methyl}pyrrolidine-1-carboxylate (130 mg, 0.31 mmol, 1.0 eq.) in ACN (2 mL) and H₂O (2 mL) was added potassium chloride (25.6 mg, 0.34 mmol, 1.1 eq.) and oxone (57.7 mg, 0.34 mmol, 1.1 eq.). The resulting mixture was stirred at 25° C. for 2 h. Desired product could be detected by LCMS. The residue was purified by reversed-phase flash chromatography to afford tert-butyl (2R,3S,4S)-3-(acetyloxy)-2-{[4-(5-chloro-1-methyl-1,2,3-triazol-4-yl)phenyl]methyl}-4-hydroxypyrrolidine-1-carboxylate (100 mg, 71.1%) as a light yellow oil. MS: m/z: Calc'd for C₂₁H₂₇ClN₄O₅ [M+H−56]⁺395; Found, 395.

Step 2: The title compound was prepared in 17.4% yield as a yellow semi-solid according to Boc Deprotection; General Procedure III in STEP 2. MS: m/z Calc'd for C₁₆H₁₉C₁N₄O₃ [M+H]⁺351; Found, 351. 1H NMR (400 MHz, Methanol-d4) δ 9.22 (s, 2H), 7.87 (d, J=7.8 Hz, 2H), 7.48 (d, J=7.9 Hz, 2H), 6.04 (d, J=3.4 Hz, 1H), 4.96 (d, J=3.4 Hz, 1H), 4.31-4.20 (m, 1H), 3.49 (s, 2H), 3.17-2.96 (m, 3H), 2.17 (s, 3H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.88.

### Synthetic Procedure of V188005

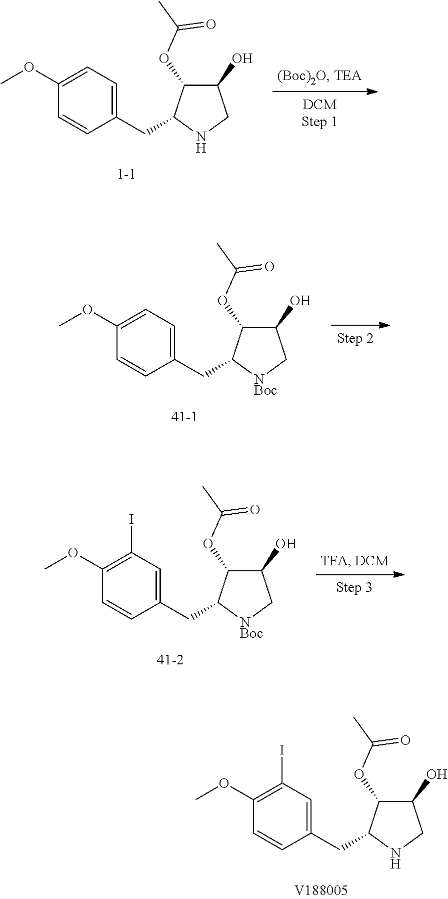 [SMILES: COc1ccc(C[C@H]2NC[C@H](O)[C@H]2OC(C)=O)cc1 ; COc1ccc(C[C@@H]2[C@H](OC(C)=O)[C@@H](O)CN2C(=O)OC(C)(C)C)cc1 ; COc1ccc(C[C@@H]2[C@H](OC(C)=O)[C@@H](O)CN2C(=O)OC(C)(C)C)cc1I ; COc1ccc(C[C@H]2NC[C@H](O)[C@H]2OC(C)=O)cc1I]

Step 1: To a stirred solution of anisomycin (1-1, 500 mg, 1.9 mmol, 1.0 eq.) in DCM (10 mL) was added Boc₂O (370.2 mg, 1.70 mmol, 0.9 eq.) and TEA (286.0 mg, 2.82 mmol, 1.5 eq.) in portions at room temperature. The resulting mixture was stirred over night at 25° C. The resulting mixture was concentrated under reduced pressure. The residue was purified by reversed-phase flash chromatography to afford tert-butyl (2R,3S,4S)-3-(acetyloxy)-4-hydroxy-2-[(4-methoxyphenyl)methyl]pyrrolidine-1-carboxylate (600 mg, 87.1%) as a light yellow oil. MS: m/z: Calc'd for C₁₉H₂₇NO₆ [M+H−56]⁺310; Found, 310.

Step 2: To a stirred solution of tert-butyl (2R,3S,4S)-3-(acetyloxy)-4-hydroxy-2-[(4-methoxyphenyl)methyl]pyrrolidine-1-carboxylate (41-1, 200 mg, 0.754 mmol, 1 eq.) in ACN (5 mL) and H₂O (5 mL) was added NIS (135.45 mg, 0.602 mmol, 1.1 eq.) and indium(iii) trifluoromethanesulfonate (30.7 mg, 0.05 mmol, 0.1 eq.) in portions at 0° C. The resulting mixture was stirred over night at 25° C. The residue was purified by reversed-phase flash chromatography to afford tert-butyl (2R,3S,4S)-3-(acetyloxy)-4-hydroxy-2-[(3-iodo-4-methoxyphenyl)methyl]pyrrolidine-1-carboxylate (150 mg, 55.8%) as a white solid. MS: m/z: Calc'd for C₁₉H₂₆INO₆ [M+H]⁺492; Found, 492.

Step 3: The title compound was prepared in 24.9% yield as a light yellow semi-solid according to Boc Deprotection; General Procedure III in STEP 2. MS: m/z Calc'd for C₁₄H₁₈INO₄ [M+H]⁺392; Found, 392. ¹H NMR (400 MHz, Methanol-d₄) δ 7.77 (d, J=2.1 Hz, 1H), 7.32 (dd, J=8.4, 2.2 Hz, 1H), 6.96 (d, J=8.4 Hz, 1H), 5.08 (d, J=3.4 Hz, 1H), 4.37 (d, J=4.3 Hz, 1H), 4.21-4.17 (m, J=8.8, 6.7, 3.4 Hz, 1H), 3.87 (s, 3H), 3.62 (dd, J=12.7, 4.4 Hz, 1H), 3.23 (d, J=12.7 Hz, 1H), 3.08 (dd, J=14.3, 6.7 Hz, 1H), 2.97 (dd, J=14.3, 8.9 Hz, 1H), 2.20 (s, 3H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 9.08.

### Synthetic Procedure of V187693

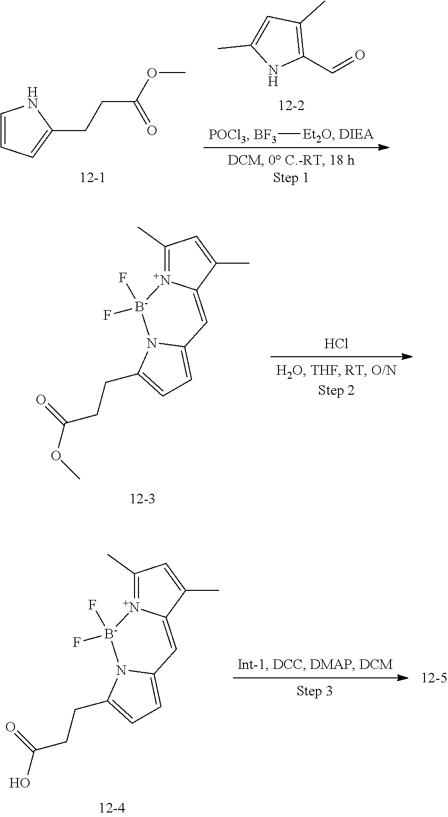

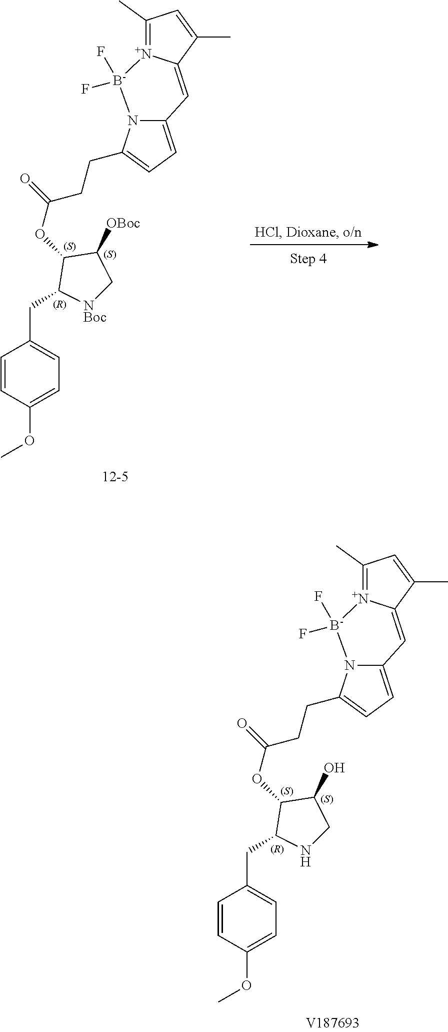

Step 1: To an ice-cooled solution of methyl 3-(1H-pyrrol-2-yl)propanoate (12-1, 500 mg, 3.26 mmol, 1 eq.) and 3,5-dimethyl-1H-pyrrole-2-carbaldehyde (12-2, 442.2 mg, 3.59 mmol, 1.1 eq.) in DCM (15 mL), was added POCl₃ (0.3 mL, 3.26 mmol, 1 eq.) dropwise. Stirring was maintained at 0° C. for 0.5 h and at room temperature for 6 h. The dark solution obtained during this time was again cooled to 0° C. and BF₃·Et₂O (1.7 mL, 13.04 mmol, 4 eq.) and DIEA (19912.6 mg, 153.87 mmol, 47.2 eq.) were added and the mixture was stirred at room temperature for an additional 12 h. LCMS showed the reaction was complete. Water was added (250 mL) to the reaction flask, and the mixture was filtered through Celite and washed with DCM. The aqueous layer was separated and extracted with DCM, dried under anhydrous sodium sulphate, filtered, concentrated. The residue was purified by silica flash chromatography using DCM as the eluent to give 2,2-difluoro-4-(3-methoxy-3-oxopropyl)-10,12-dimethyl-1lambda5,3-diaza-2-boratricyclo[7.3.0.0∧{3,7}]dodeca-1(12),4,6,8,10-pentaen-1-ylium-2-uide (578 mg, 56.9%) as a brown solid (0.646 g, yield 71%). MS: m/z: Calc'd for C₁₅H₁₇BF₂N₂O₂ [M−F]+287, found 287; [M−H]⁻305, found 305.

Step 2: To a solution of 2,2-difluoro-4-(3-methoxy-3-oxopropyl)-10,12-dimethyl-1lambda5,3-diaza-2-boratricyclo[7.3.0.0{3,7}]dodeca-1(12),4,6,8,10-pentaen-1-ylium-2-uide (12-3, 500 mg, 1.63 mmol, 1 eq.) in THF (75 mL) was added H₂O (50 mL) and conc·HCl (30 mL). The mixture was stirred at room temperature for 40 h. After completion of the reaction monitored by LCMS, DCM (300 mL) was added and the phases separated. The aqueous layer was then extracted with DCM (2×300 mL) and finally the combined organic layers were washed with brine and dried under vacuum. The crude product was purified by silica flash chromatography using DCM:MeOH (10:1) as the eluent to give 4-(2-carboxyethyl)-2,2-difluoro-10,12-dimethyl-1lambda5,3-diaza-2-boratricyclo [7.3.0.0∧{3,7}]dodeca-1(12),4,6,8,10-pentaen-1-ylium-2-uide (255 mg, 53.4%) as a red solid. MS: m/z: Calc'd for C₁₄H₁₅BF₂N₂O₂ [M−F]+273, found 273.

Step 3 & 4: The title compound was prepared in 31.3% overall yield as a red solid according to Condensation Reaction with DCC and DMAP; General Procedure I using 4-(2-carboxyethyl)-2,2-difluoro-10,12-dimethyl-1lambda5,3-diaza-2-boratricyclo[7.3.0.0∧{3,7}]dodeca-1(12),4,6,8,10-pentaen-1-ylium-2-uide (12-4) in STEP 3; Boc Deprotection; General Procedure III using HCl (4 M in dioxane) instead of TFA in STEP 4. MS: m/z Calc'd for C₂₆H₃₀BF₂N₃O₄ [M+H]⁺498, found 498.

¹H NMR (400 MHz, Methanol-d₄) δ 7.47 (s, 1H), 7.21-7.15 (m, 2H), 7.04 (d, J=4.0 Hz, 1H), 6.94-6.86 (m, 2H), 6.39 (d, J=4.0 Hz, 1H), 6.27 (s, 1H), 5.09 (d, J=3.4 Hz, 1H), 4.33 (d, J=4.1 Hz, 1H), 4.23-4.14 (m, 1H), 3.79 (s, 3H), 3.55 (dd, J=12.7, 4.3 Hz, 1H), 3.30 (d, J=6.8 Hz, 2H), 3.20 (d, J=12.7 Hz, 1H), 3.06-2.85 (m, 4H), 2.55 (s, 3H), 2.30 (s, 3H). Prep-HPLC purification conditions: Column: Xselect CSH Prep C18 Column, 30*150 mm, 5 μm; Mobile Phase A: Water (0.05% HCl), Mobile Phase B: ACN; Flow rate: 60 mL/min; Gradient: 15% B to 45% B in 10 min; Wave Length: 254 nm/220 nm nm; RT1(min): 8.68.

### Synthetic Procedure of V187845

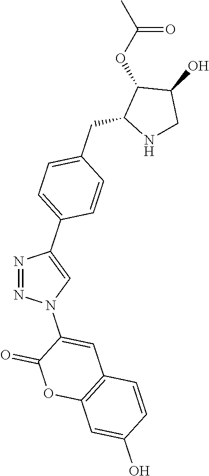 [SMILES: CC(=O)O[C@@H]1[C@@H](O)CN[C@@H]1Cc1ccc(-c2cn(-c3cc4ccc(O)cc4oc3=O)nn2)cc1]

The title compound was prepared in 39.3% overall yield as a yellow solid solid according to Cyclization reaction for synthesis of Substituted triazoles; General Procedure VI using 3-azido-7-hydroxychromen-2-one in STEP 1; Boc Deprotection; General Procedure III in STEP 2. MS: m/z Calc'd for C₂₄H₂₂N₄O₆ [M+H−56]⁺463; found 463. ¹H NMR (400 MHz, Methanol-d₄) δ 8.91 (d, J=1.1 Hz, 1H), 8.57 (d, J=1.6 Hz, 1H), 7.98-7.91 (m, 2H), 7.69 (d, J=8.5 Hz, 1H), 7.46 (d, J=8.0 Hz, 2H), 6.94 (dd, J=8.6, 2.3 Hz, 1H), 6.86 (d, J=2.2 Hz, 1H), 5.18-5.13 (m, 1H), 4.43-4.37 (m, 1H), 4.30-4.21 (m, 1H), 3.64 (dd, J=12.7, 4.4 Hz, 1H), 3.23 (dd, J=13.7, 6.8 Hz, 2H), 3.09 (dd, J=14.3, 9.0 Hz, 1H), 2.22 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 11% B to 32% B in 10 min; Wave Length: 254 nm/220 nm nm; RT1(min): 8.68.

### Synthetic Procedure of V186713

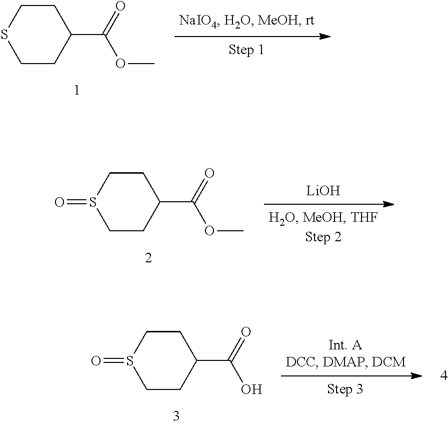

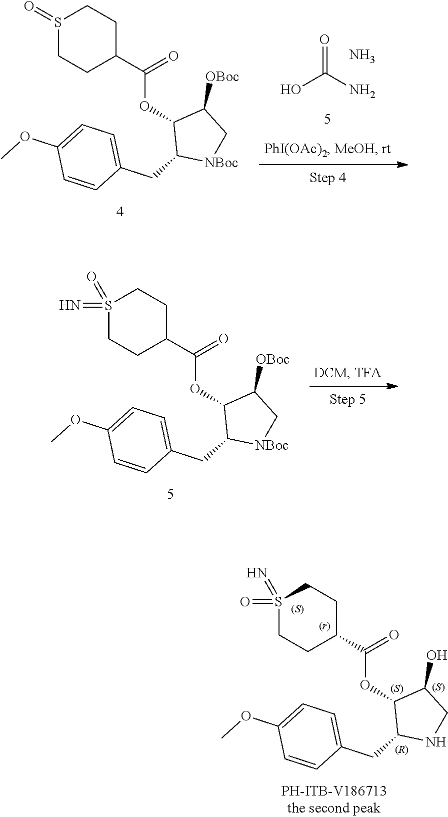

Step 1: A solution of methyl tetrahydro-2H-thiopyran-4-carboxylate (2.00 g, 12.5 mmol) in MeOH (120 mL) was treated with a solution of sodium periodate (2.94 g, 13.7 mmol) in water (30 mL), and the resulting mixture was stirred at rt. After 18.5 h, the mixture was filtered and the collected solid was rinsed with MeOH. The filtrate was concentrated to remove most of the methanol, and the resulting aqueous phase was treated with solid NaCl and extracted 3 times with EtOAc. The combined organic phases were dried and concentrated to provide a mixture of cis and trans isomers of methyl tetrahydro-2H-thiopyran-4-carboxylate 1-oxide (948 mg, 43%). MS: m/z: Calc'd for C₇H₁₂O₃S [M+H]⁺177, found [M+H]⁺177.

Step 2: To a stirred solution of methyl 1-oxo-1lambda4-thiane-4-carboxylate (500 mg, 2.837 mmol, 1.0 eq.) in THF (4 mL), MeOH (2 mL) and H₂O (2 mL) was added LiOH (203.9 mg, 8.51 mmol, 3.0 eq.) at room temperature. The resulting mixture was stirred over night at 25° C. The reaction mixture was quenched with water and extracted with EA. The extracts were washed with brine, dried over anhydrous sodium sulfate, filtered and concentrated. The residue was purified by flash to obtained 1-oxo-1lambda4-thiane-4-carboxylic acid (200 mg, 43.5%) as a light yellow oil. MS: m/z: Calc'd for C₆H₁₀O₃S [M+H]⁺163, found [M+H]⁺163.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.A, 120 mg, 0.28 mmol, 1.0 eq.) and 1-oxo-1lambda4-thiane-4-carboxylic acid (91.9 mg, 0.57 mmol, 2.0 eq.) in DCM (5 mL) were added DCC (87.7 mg, 0.42 mmol, 1.5 eq.) and DMAP (34.6 mg, 0.28 mmol, 1.0 eq.) at 0° C. The resulting reaction mixture was stirred at ambient temperature for overnight. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-2-[(4-methoxyphenyl)methyl]-3-(1-oxo-1lambda4-thiane-4-carbonyloxy)pyrrolidine-1-carboxylate (160 mg, 99.5%) as a light yellow oil. MS: m/z: Calc'd for C₂₈H₄₁NO₉S [M+H]⁺568, found [M+H]⁺568.

Step 4: A solution of tert-butyl (2R,3S,4S)-4-[(tert-butoxycarbonyl)oxy]-2-[(4-methoxyphenyl)methyl]-3-(1-oxo-1lambda4-thiane-4-carbonyloxy)pyrrolidine-1-carboxylate (160 mg, 0.28 mmol, 1.0 eq.), ammonium carbamate (165 mg, 2.12 mmol, 7.5 eq.) and DIB (408.5 mg, 1.27 mmol, 4.5 eq.) in MeOH (5 mL) was stirred for 2 h at room temperature under nitrogen atmosphere. Desired product could be detected by LCMS. The crude product was purified by reverse phase flash with the following conditions (Column: Xselect CSH Prep Fluoro-Phenyl Column, 19*250 mm, 5 μm; Mobile Phase A: Water(0.05% TFA), Mobile Phase B: ACN; Flow rate: 25 mL/min mL/min; Gradient: 25% B to 45% B in 10 min; Wave Length: 254 nm/220 nm nm; RT1(min): 9.47) to afford tert-butyl (2R,3S,4S)-4-[(tert-butoxycarbonyl)oxy]-3-(1-imino-1-oxo-1lambda6-thiane-4-carbonyloxy)-2-[(4-methoxyphenyl)methyl]pyrrolidine-1-carboxylate (140 mg, 85.2%) as a white solid. MS: m/z: Calc'd for C₂₈H₄₂N₂O₉S [M+H]⁺583, found [M+H]⁺583.

Step 5: To a stirred solution of tert-butyl (2R,3S,4S)-4-[(tert-butoxycarbonyl)oxy]-3-(1-imino-1-oxo-1lambda6-thiane-4-carbonyloxy)-2-[(4-methoxyphenyl)methyl]pyrrolidine-1-carboxylate (135 mg, 0.23 mmol, 1.0 eq.) in DCM (2 mL) was added TFA (1 mL) in portions at 0° C. under nitrogen atmosphere. The resulting mixture was stirred for 1 h at room temperature under nitrogen atmosphere. Desired product could be detected by LCMS. The crude product (135 mg) was purified by Prep-HPLC with the following conditions (Column: Xselect CSH Prep C18 C18 Column, 30*150 mm, 5 μm; Mobile Phase A: Water(0.05% TFA), Mobile Phase B: ACN; Flow rate: 60 mL/min mL/min; Gradient: 2% B to 6% B in 10 min; Wave Length: 254 nm/220 nm nm; RT1(min): 8.5) to afford (2R,3S,4S)-4-hydroxy-2-[(4-methoxyphenyl)methyl]pyrrolidin-3-yl (1 r)-1-imino-1-oxo-1lambda6-thiane-4-carboxylate; trifluoroacetic acid (19.8 mg, 17.20%) as a white solid. MS: m/z: Calc'd for C₁₈H₂₆N₂O₅S [M+H]⁺383, found [M+H]⁺383. ¹H NMR (400 MHz, DMSO-d6) δ 7.22 (d, J=8.3 Hz, 2H), 6.96-6.88 (m, 2H), 4.95 (d, J=3.3 Hz, 1H), 4.29 (d, J=4.3 Hz, 1H), 4.04-3.99 (m, 1H), 3.74 (s, 3H), 3.67 (s, 1H), 3.57-3.43 (m, 5H), 3.08 (d, J=12.7 Hz, 1H), 3.04-2.85 (m, 3H), 2.41-2.32 (m, 2H), 2.23-2.10 (m, 2H).

### Example 3: Synthesis of Additional Compounds of the Present Disclosure

### General Procedure I: Boc Deprotection

The corresponding Boc-protected amine (1 equiv) was dissolved in anhydrous CH₂Cl₂ (5 mL/mmol), and TFA (5 mL/mmol) was added. The mixture was stirred at r.t. for 2 h. After removal of the volatiles, the oily residue was treated with toluene to azeotropically remove the TFA residue. The residue was purified by prep-HPLC.

General Procedure II: Condensation Reaction with DCC, DMAP for Synthesis of Ester

To a stirred mixture of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure, 60 mg, 1.00 equiv) in DCM (5 mL) were added corresponding acid (2 equiv), DMAP (1 equiv) and N-(N-cyclohexylcarboximidoyl)cyclohexanamine (1.5 equiv). The resulting mixture was stirred at room temperature for 2 h. Upon completion, the mixture concentrated under reduced pressure. The residue was dissolved in MeCN (5 mL). The residue was purified by reversed-phase flash chromatography.

### Synthetic Preparation of (2R,3S,4S)-4-hydroxy-2-{[4-(trifluoromethoxy)phenyl]methyl}pyrrolidin-3-yl acetate (V187185

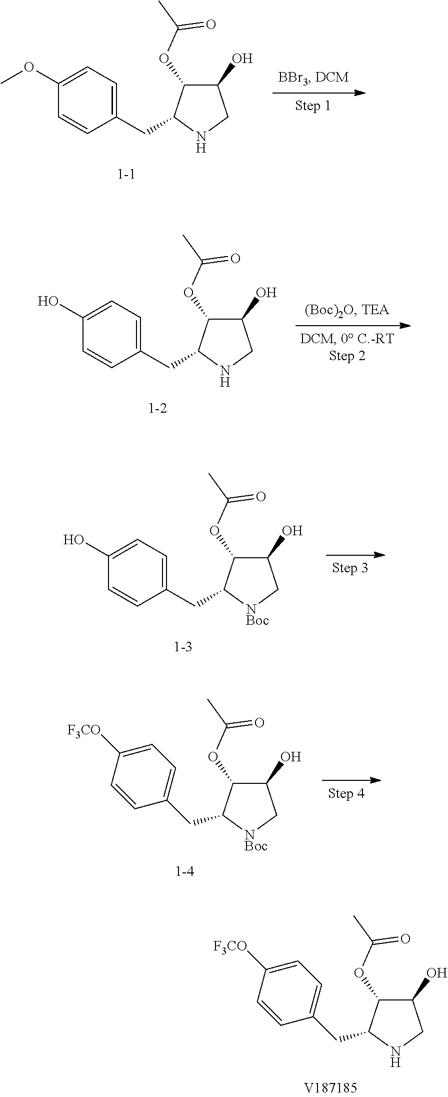 [SMILES: COc1ccc(C[C@H]2NC[C@H](O)[C@H]2OC(C)=O)cc1 ; CC(=O)O[C@@H]1[C@@H](O)CN[C@@H]1Cc1ccc(O)cc1 ; CC(=O)O[C@H]1[C@@H](Cc2ccc(O)cc2)N(C(=O)OC(C)(C)C)C[C@@H]1O ; CC(=O)O[C@H]1[C@@H](Cc2ccc(OC(F)(F)F)cc2)N(C(=O)OC(C)(C)C)C[C@@H]1O ; CC(=O)O[C@@H]1[C@@H](O)CN[C@@H]1Cc1ccc(OC(F)(F)F)cc1]

Step 1: To a stirred solution of anisomycin (1-1, 1.5 g, 5.65 mmol, 1 equiv) in Dichloromethane (8 mL) was added Boron tribromide (16.8 mL, 3 equiv) at −78° C. dropwise. The reaction mixture was stirred at −78° C. for another 2 h and warmed to room temperature 1 h. Upon completion, the reaction mixture was quenched by saturated NaHCO₃ solution. The DCM was removed, and the solution was lyophilized to obtain (2R,3S,4S)-4-hydroxy-2-[(4-hydroxyphenyl)methyl]pyrrolidin-3-yl acetate (1.8 g, 85.41%) as a crude. MS: m/z: Calc'd for C₁₃H₁₇NO₄ [M+H]⁺252; found 252.

Step 2: To a stirred solution of (2R,3S,4S)-4-hydroxy-2-[(4-hydroxyphenyl)methyl]pyrrolidin-3-yl acetate (1-2, 1.8 g, 7.16 mmol, 1 equiv) and triethylamine (2.54 g, 25.07 mmol, 3.5 equiv) in DCM (30 mL) was added di-tert-butyl dicarbonate (1.88 g, 8.59 mmol, 1.2 equiv) at 0° C. The mixture was stirred at room temperature for 3 h. After completion of the reaction monitored by LCMS, the reaction mixture was filtrated. The filtrate was concentrated. The residue was purified by a reversed-phase column to obtain tert-butyl (2R,3S,4S)-3-(acetyloxy)-4-hydroxy-2-[(4-hydroxyphenyl)methyl]pyrrolidine-1-carboxylate (1.7 g, 67.54%) as a white solid. MS: m/z: Calc'd for C₁₈H₂₅NO₆ [M−H]⁻350; found 350.

Step 3: To a stirred mixture of tert-butyl (2R,3S,4S)-3-(acetyloxy)-4-hydroxy-2-[(4-hydroxyphenyl)methyl]pyrrolidine-1-carboxylate (1-3, 100 mg, 0.25 mmol, 1 equiv), trimethyl(trifluoromethyl)silane (202.3 mg, 1.42 mmol, 5 equiv), Silver trifluoromethanesulfonate (365.9 mg, 1.42 mmol, 5 equiv), 6-fluoropyridine-3-carbonitrile (173.7 mg, 1.45 mmol, 5 equiv), 4-(chloromethyl)-1-fluoro-1,4-diazabicyclo[2.2.2]octane-1,4-diium (201.6 mg, 0.57 mmol, 2 equiv) and N-(benzenesulfonyl)-S-phenylfluoranesulfonamido (179.8 mg, 0.57 mmol, 2 equiv) in Toluene (12 mL) and (trifluoromethyl)benzene (3 mL) was added CsF (259.7 mg, 1.710 mmol, 6 equiv) at room temperature under N₂. The resulting mixture was stirred at room temperature for overnight. Upon completion, the mixture was filtered, the filter cake was washed with Toluene (12 mL). The filtrate was concentrated under reduced pressure. The residue was purified by reversed-phase flash chromatography to afford tert-butyl (2R,3S,4S)-3-(acetyloxy)-4-hydroxy-2-{[4-(trifluoromethoxy)phenyl]methyl}pyrrolidine-1-carboxylate (100 mg, 90% purity) as a white solid.

MS: m/z: Calc'd for C₁₉H₂₄F₃NO₆ [M+H⁺22]⁺442, found 442.

Step 4: The title compound was prepared in 51.1% yield as a white solid according to Boc Deprotection; General Procedure I. MS: m/z Calc'd for C₁₄H₁₆F₃NO₄ [M+H]⁺320, found 320. ¹H NMR (400 MHz, Methanol-d₄) δ 7.49-7.41 (m, 2H), 7.35-7.28 (m, 2H), 5.16-5.10 (m, 1H), 4.47-4.36 (m, 1H), 4.30-4.21 (m, 1H), 3.62 (dd, J=12.7, 4.4 Hz, 1H), 3.27-3.16 (m, 2H), 3.07-3.05 (m, 1H), 2.20 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.5.

### Synthetic Preparation of (2R,3S,4S)-2-({4-[1-(2-aminopyrimidin-4-yl)-1,2,3-triazol-4-yl]phenyl}methyl)-4-hydroxypyrrolidin-3-yl acetate (V188057

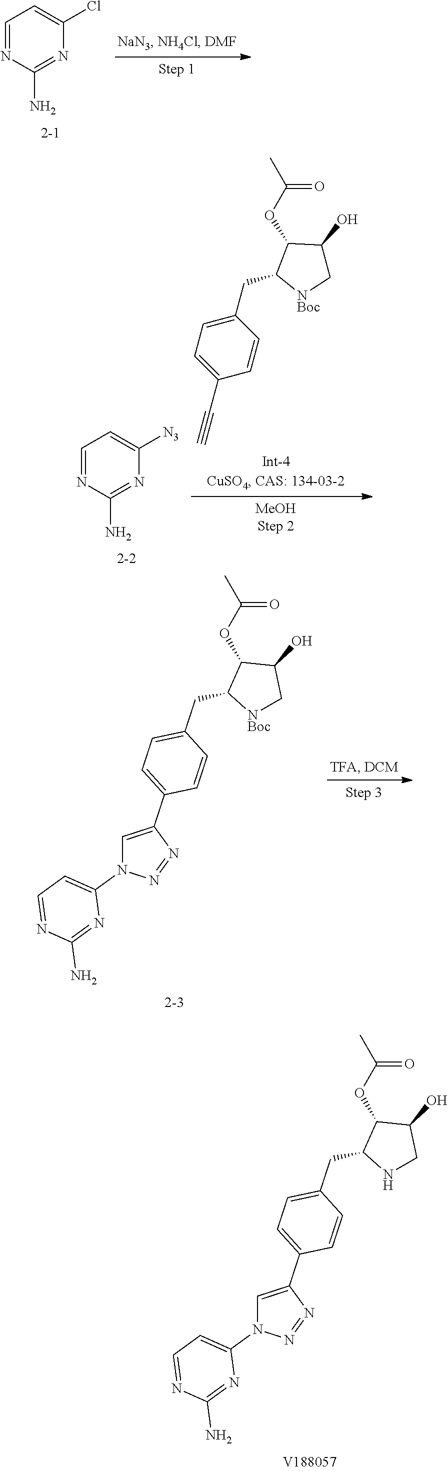

Step 1: To a stirred solution of 4-chloropyrimidin-2-amine (2-1, 500 mg, 3.860 mmol, 1 equiv) in ACN (30 mL) was added NaN₃ (501.82 mg, 7.72 mmol, 2 eq.) and NH₄Cl (412.89 mg, 7.72 mmol, 2 eq.) in portions at room temperature. The resulting mixture was stirred overnight at 80° C.

Desired product could be detected by LCMS. The reaction was quenched by the addition of Water (10 mL) at 0° C. The aqueous layer was extracted with EtOAc (3×5 mL). Combined the organic phase, dried over anhydrous sodium sulphate, filtered, concentrated to afford 4-azidopyrimidin-2-amine (300 mg, 57.1%) as a light yellow oil which was used directly in the next step without further purification.

MS: m/z: Calc'd for C₄H₄N₆ [M+H]⁺137; Found, 137.

Step 2: To a stirred solution of tert-butyl (2R,3S,4S)-3-(acetyloxy)-2-[(4-ethynylphenyl)methyl]-4-hydroxypyrrolidine-1-carboxylate (Int-4, preparation in carbamate procedure, 150 mg, 0.42 mmol, 1 eq.) and 4-azidopyrimidin-2-amine (56.8 mg, 0.42 mmol, 1 eq.) in MeOH (5 mL) was added CuSO_{4·5}H₂O (104.2 mg, 0.42 mmol, 1 eq.) and sodium (5R)-5-[(1S)-1,2-dihydroxyethyl]-3,4-dihydroxy-2,5-dihydrofuran-2-one (166.2 mg, 0.83 mmol, 2 eq.) in portions at room temperature. The resulting mixture was stirred over night at 25° C. Desired product could be detected by LCMS. The residue was purified by reversed-phase flash chromatography to afford tert-butyl (2R,3S,4S)-3-(acetyloxy)-2-({4-[1-(2-aminopyrimidin-4-yl)-1,2,3-triazol-4-yl]phenyl}methyl)-4-hydroxypyrrolidine-1-carboxylate (90 mg, 43.5%) as a light yellow oil. MS: m/z: Calc'd for C₂₄H₂₉N₇O₅[M+H−56]⁺440; Found, 440.

Step 3: The title compound was prepared in 23.8% yield as a white solid according to Boc Deprotection; General Procedure I. MS: m/z Calc'd for C₁₉H₂₁N₇O₃ [M+H]⁺396; Found, 396. ¹H NMR (400 MHz, Methanol-d₄) δ 9.14-9.08 (m, 1H), 8.51-8.45 (m, 1H), 8.02-7.95 (m, 2H), 7.51-7.41 (m, 3H), 5.15 (d, J=3.4 Hz, 1H), 4.41 (d, J=4.5 Hz, 1H), 4.35-4.26 (m, 1H), 3.70-3.60 (m, 1H), 3.29-3.19 (m, 2H), 3.17-3.06 (m, 1H), 2.23 (s, 3H).

Prep-HPLC purification conditions: Column: Xselect CSH Prep C18 Column, 30*150 mm, 5 μm; Mobile Phase A: Water (0.1% FA), Mobile Phase B: ACN; Flow rate: 60 mL/min; Gradient: 2% B to 22% B in 10 min; Wave Length: 254 nm/220 nm; RT1(min): 8.68.

### Synthetic Preparation of (2R,3S,4S)-4-hydroxy-2-[(4-nitrophenyl)methyl]pyrrolidin-3-yl acetate (V188168

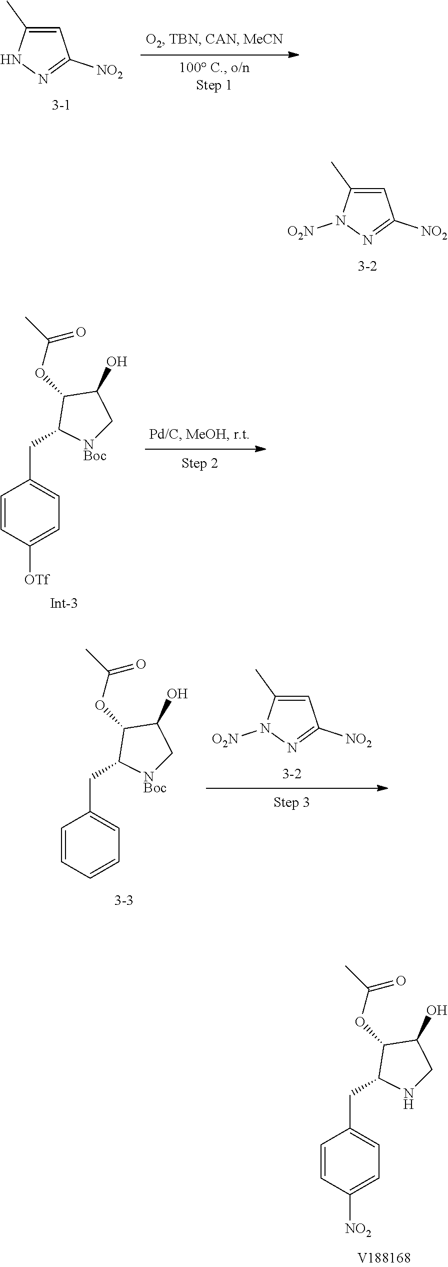

Step 1: To a stirred mixture of 3-methyl-5-nitro-2H-pyrazole (3-1, 500 mg, 3.94 mmol, 1 equiv) and tert-butyl nitrite (405.6 mg, 3.93 mmol, 1 equiv) in ACN (10 mL) was added CAN (4329.4 mg, 7.88 mmol, 2 equiv) at room temperature under oxygen atmosphere. The resulting mixture was stirred for overnight at 100° C. under oxygen atmosphere. The reaction mixture was quenched with water and extracted with EA. The extracts were washed with brine, dried over anhydrous sodium sulfate, filtered and concentrated. The residue was purified by silica gel column chromatography, eluted with PE/EA (5:1) to afford 5-methyl-1,3-dinitropyrazole (100 mg, 14.7%) as a yellow oil.

Step 2: To a stirred mixture of tert-butyl (2R,3S,4S)-3-(acetyloxy)-4-hydroxy-2-{[4-(trifluoromethanesulfonyloxy)phenyl]methyl}pyrrolidine-1-carboxylate (Int-3, preparation in carbamate procedure, 300 mg, 0.61 mmol, 1 equiv) in MeOH (10 mL) was added Pd/C (165.9 mg, 1.52 mmol, 2.5 equiv) at room temperature under nitrogen atmosphere. Subsequently, hydrogen was introduced into the aforementioned mixture, which was then stirred continuously overnight at ambient temperature. The resulting mixture was filtered, the filter cake was washed with MeOH (2×2 mL).

The filtrate was concentrated under reduced pressure. This resulted in tert-butyl (2R,3S,4S)-3-(acetyloxy)-2-benzyl-4-hydroxypyrrolidine-1-carboxylate (180 mg, 86.9%) as a yellow oil. MS: m/z: Calc'd for C₁₈H₂₅NO₅ [M+H−56]⁺280, found 280.

Step 3: To a stirred mixture of 5-methyl-1,3-dinitropyrazole (3-2, 102.2 mg, 0.56 mmol, 2 equiv) and tert-butyl (2R,3S,4S)-3-(acetyloxy)-2-benzyl-4-hydroxypyrrolidine-1-carboxylate (3-3, 100 mg, 0.28 mmol, 1.00 equiv) in HFIP (10 mL) was added Indium(III) trifluoromethanesulfonate (33.5 mg, 0.06 mmol, 0.2 equiv) at room temperature under nitrogen atmosphere. The resulting mixture was stirred at 80° C. for overnight under nitrogen atmosphere. The reaction mixture was quenched with water and extracted with EA. The extracts were washed with brine, dried over anhydrous sodium sulfate, filtered and concentrated. The crude product (20 mg) was purified by Prep-CHIRAL-HPLC to afford (2R,3S,4S)-4-hydroxy-2-[(4-nitrophenyl)methyl]pyrrolidin-3-yl acetate; trifluoroacetic acid (2.3 mg, 98.2% purity) as a white solid. MS: m/z: Calc'd for C₁₃H₁₆N₂O₅ [M+H]⁺281, found 281. 1H NMR (400 MHz, MeOD) δ 8.21-8.13 (m, 2H), 7.53-7.45 (m, 2H), 4.18-4.11 (m, 1H), 3.69-3.59 (m, 1H), 3.39-3.31 (m, 2H), 3.05-2.88 (m, 2H), 2.73 (dd, J=12.2, 2.6 Hz, 1H), 2.12 (s, 3H).

Prep-HPLC purification conditions: Column: CHIRAL ART Cellulose-SB, 2*25 cm, 5 μm; Mobile Phase A: Hex (0.5% 2M NH₃-MeOH)—HPLC, Mobile Phase B: MeOH: DCM=1:1—HPLC; Flow rate: 20 ML/MIN mL/min; Gradient: isocratic 15; Wave Length: 254/220 nm nm; RT1(min): 11.358; RT2(min): 13.201; Sample Solvent: EtOH—HPLC; Injection Volume: 1.0 mL; Number Of Runs: 1.

### Synthetic Preparation of (2R,3S,4S)-4-hydroxy-2-[(4-methoxyphenyl)methyl]-4-methylpyrrolidin-3-yl acetate (V188182

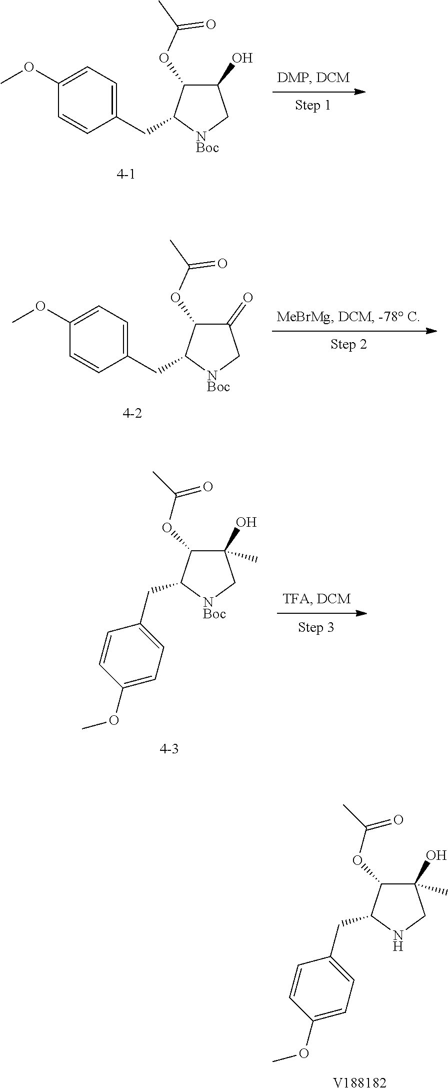

Step 1: To the solution of tert-butyl (2R,3S,4S)-3-(acetyloxy)-4-hydroxy-2-[(4-methoxyphenyl)methyl]pyrrolidine-1-carboxylate (4-1, 200 mg, 0.547 mmol, 1 equiv) in DCM (5 mL) was added DMP (278.56 mg, 0.656 mmol, 1.2 equiv). The mixture was stirred at rt for 1 hour. The mixture was filtered and the filtrate was concentrated to give the crude product which was purified by Prep TLC. The tert-butyl (2R,3S)-3-(acetyloxy)-2-[(4-methoxyphenyl)methyl]-4-oxopyrrolidine-1-carboxylate (180 mg, 90.5%) was obtained as a white solid. MS: m/z: Calc'd for C₁₉H₂₅NO₆ [M+H−56]⁺349, found 349.

Step 2: To the solution of tert-butyl (2R,3S)-3-(acetyloxy)-2-[(4-methoxyphenyl)methyl]-4-oxopyrrolidine-1-carboxylate (4-2, 100 mg, 0.28 mmol, 1 equiv) in DCM (5 mL) was added MeMgBr (98.44 mg, 0.825 mmol, 3 equiv) dropwise at −78° C. under N₂. The mixture was stirred at −78° C. for 0.5 h. The reaction was quenched with 1 M HCl. The mixture was diluted with EA. The organic layer was washed with water, dried and concentrated to give the crude product which was used in the next step without further purification. The tert-butyl (2R,3S,4S)-3-(acetyloxy)-4-hydroxy-2-[(4-methoxyphenyl)methyl]-4-methylpyrrolidine-1-carboxylate (80 mg, 76.6%) was obtained as a brown oil. MS: m/z: Calc'd for C₂₀H₂₉NO₆[M+H−56]⁺324, found 324.

Step 3: The title compound was prepared in 29.5% yield as a black oil according to Boc Deprotection; General Procedure I. MS: m/z Calc'd for C₁₅H₂₁NO₄[M+H]⁺280, found 280. ¹H NMR (400 MHz, Methanol-d₄) δ 7.25-7.19 (m, 2H), 6.96-6.89 (m, 2H), 5.14 (d, J=6.0 Hz, 1H), 4.22-4.12 (m, 1H), 3.79 (s, 3H), 3.29 (d, J=11.8 Hz, 1H), 3.17 (d, J=11.9 Hz, 1H), 3.11-3.02 (m, 1H), 3.00-2.89 (m, 1H), 2.20 (s, 3H), 1.45 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 9.3.

### Synthetic Preparation of (2R,3S,4S)-4-hydroxy-2-{[4-(1,3-oxazol-5-yl)phenyl]methyl}pyrrolidin-3-yl 2-(3,3-difluorocyclobutyl)acetate (V192387

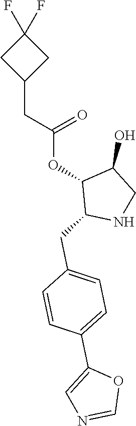 [SMILES: O=C(CC1CC(F)(F)C1)O[C@@H]1[C@@H](O)CN[C@@H]1Cc1ccc(-c2cnco2)cc1]

The title compound was prepared in 31.7% overall yield as a white solid according to Condensation reaction with DCC, DMAP for synthesis of ester; General Procedure II using (3,3-difluorocyclobutyl)acetic acid in STEP 1; Boc Deprotection; General Procedure I in STEP 2. MS: m/z Calc'd for C₃₀H₃₈F₂N₂O₈ [M+H]⁺593, found 593. ¹H NMR (400 MHz, Methanol-d₄) δ 8.27 (s, 1H), 7.81-7.73 (m, 2H), 7.55 (s, 1H), 7.48-7.41 (m, 2H), 5.19-5.13 (m, 1H), 4.41-4.35 (m, 1H), 4.35-4.26 (m, 1H), 3.63 (dd, J=12.7, 4.4 Hz, 1H), 3.30-3.16 (m, 2H), 3.09 (dd, J=14.3, 8.9 Hz, 1H), 2.88-2.68 (m, 4H), 2.64-2.52 (m, 1H), 2.44-2.23 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 16% B to 37% B in 10 min; Wave Length: 254 nm/220 nm; RT1(min): 8.5.

### Synthetic Preparation of (2R,3S,4S)-4-hydroxy-2-{[4-(1,3-thiazol-5-yl)phenyl]methyl}pyrrolidin-3-yl 2-(3,3-difluorocyclobutyl)acetate (V192840

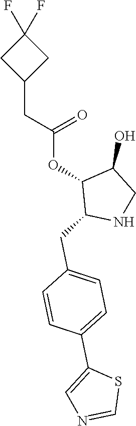 [SMILES: O=C(CC1CC(F)(F)C1)O[C@@H]1[C@@H](O)CN[C@@H]1Cc1ccc(-c2cncs2)cc1]

The title compound was prepared in 33.8% overall yield as a white solid according to Condensation reaction with DCC, DMAP for synthesis of ester; General Procedure II using tert-butyl (2R,3S,4S)-4-((tert-butoxycarbonyl)oxy)-3-hydroxy-2-(4-(thiazol-5-yl)benzyl)pyrrolidine-1-carboxylate (Int-12, preparation in carbamate procedure) instead of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure), (3,3-difluorocyclobutyl)acetic acid in STEP 1; Boc Deprotection; General Procedure I in STEP 2. MS: m/z Calc'd for C₂₀H₂₂F₂N₂O₃S [M+H]⁺409, found 409. ¹H NMR (400 MHz, Methanol-d₄) δ 9.00 (s, 1H), 8.20 (d, J=0.7 Hz, 1H), 7.75-7.67 (m, 2H), 7.46-7.39 (m, 2H), 5.17 (d, J=1.2 Hz, 1H), 4.39 (d, J=4.5 Hz, 1H), 4.31 (dd, J=9.6, 6.5 Hz, 1H), 3.62 (dd, J=12.7, 4.4 Hz, 1H), 3.30-3.16 (m, 2H), 3.07 (dd, J=14.4, 9.0 Hz, 1H), 2.83-2.68 (m, 4H), 2.62 (s, 1H), 2.42-2.28 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 16% B to 46% B in 10 min; Wave Length: 254 nm/220 nm; RT1(min): 8.5.

### Synthetic Preparation of (2R,3S,4S)-4-hydroxy-2-{[4-(1,3-thiazol-5-yl)phenyl]methyl}pyrrolidin-3-yl 2-(3,3-difluorocyclobutyl)acetate (V192841

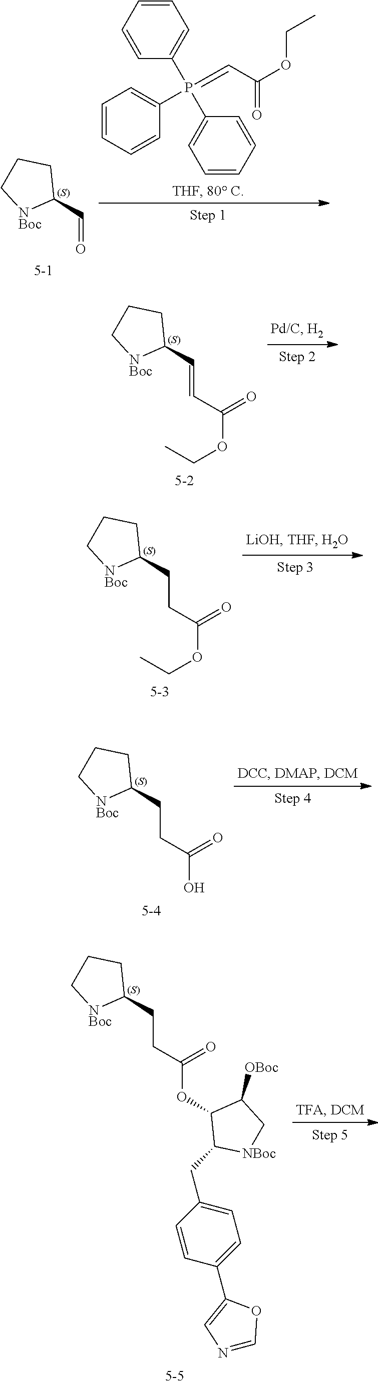

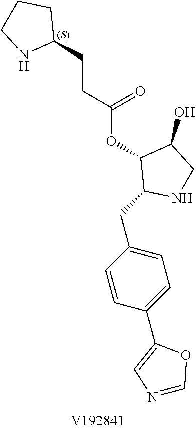

Step 1: To a stirred mixture of ethyl 2-(triphenyl-lambda5-phosphanylidene)acetate (5-1, 1.8 g, 5.02 mmol, 1 equiv) in THF (10 mL) was added tert-butyl (2S)-2-formylpyrrolidine-1-carboxylate (1 g, 5.02 mmol, 1 equiv) at room temperature. The resulting mixture was stirred at room temperature for overnight. Upon completion, concentrated, the residue was purified by reversed-phase flash chromatography to afford tert-butyl (2S)-2-(3-ethoxy-3-oxoprop-1-en-1-yl)pyrrolidine-1-carboxylate (1.2 g, 88.8% yield, 95% purity) as a yellow oil. MS: m/z: Calc'd for C₁₄H₂₃NO₄ [M+H−56]⁺214, found 214.

Step 2: To a stirred solution of tert-butyl (2S)-2-(3-ethoxy-3-oxoprop-1-en-1-yl)pyrrolidine-1-carboxylate (5-2, 1.3 g, 4.83 mmol, 1 equiv) in EtOH (25 mL) was added Pd/C (300 mg) at room temperature under nitrogen atmosphere. The resulting mixture was stirred at room temperature for 2 h under hydrogen atmosphere. After completion of reaction monitored by LCMS. Filtered and washed with MeOH (3×30 mL). The filtrate was concentrated to afford tert-butyl (2S)-2-(3-ethoxy-3-oxopropyl)pyrrolidine-1-carboxylate (1.16 g) as a colorless oil which was used directly in the next step without further purification. MS: m/z: Calc'd for C₁₄H₂₃NO₄ [M+H]⁺272, found 272.

Step 3: To a stirred solution of tert-butyl (2S)-2-(3-ethoxy-3-oxopropyl)pyrrolidine-1-carboxylate (5-3, 1.1 g, 4.05 mmol, 1 equiv) in THF (10 mL) was added LiOH (291.3 mg, 12.16 mmol, 3 equiv) in H₂O (5 mL) and EtOH (5 mL) at room temperature. The resulting mixture was stirred at room temperature for 4 h. After completion of reaction monitored by LCMS. The residue was purified by flash to afford 3-[(2S)-1-(tert-butoxycarbonyl)pyrrolidin-2-yl]propanoic acid (980 mg, 99.4% yield, 98% purity) as a yellow solid. MS: m/z: Calc'd for Chemical Formula: C₁₂H₂₁NO₄ [M+H−56]⁺188, found 188.

### Step 4 and 5

The title compound was prepared in 37.2% overall yield as an off-white solid according to Condensation reaction with DCC, DMAP for synthesis of ester; General Procedure II using 3-[(2S)-1-(tert-butoxycarbonyl)pyrrolidin-2-yl]propanoic acid (5-4) in STEP 4; Boc Deprotection; General Procedure I in STEP 5. MS: m/z Calc'd for C₂₁H₂₇N₃O₄ [M+H]⁺386, found 386. ¹H NMR (400 MHz, Methanol-d₄) δ 8.27 (s, 1H), 7.80-7.73 (m, 2H), 7.54 (s, 1H), 7.46 (d, J=8.0 Hz, 2H), 5.19 (s, 1H), 4.41 (d, J=4.2 Hz, 1H), 4.31 (s, 1H), 3.71-3.55 (m, 2H), 3.40-3.33 (m, 2H), 3.29-3.17 (m, 2H), 3.17-3.07 (m, 1H), 2.75-2.62 (m, 2H), 2.34-2.22 (m, 1H), 2.22-1.97 (m, 4H), 1.79-1.65 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 26% B to 56% B in 10 min; Wave Length: 254 nm/220 nm; RT1(min): 8.5.

### Synthetic Preparation of (2R,3S,4S)-4-hydroxy-2-{[4-(1,3-thiazol-5-yl)phenyl]methyl}pyrrolidin-3-yl 3-[(2S)-pyrrolidin-2-yl]propanoate (V192842

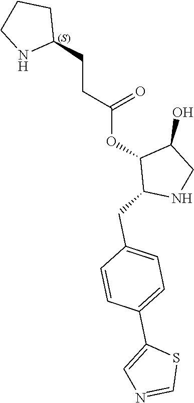

The title compound was prepared in 41.9% overall yield as an off-white solid according to Condensation reaction with DCC, DMAP for synthesis of ester; General Procedure II using tert-butyl (2R,3S,4S)-4-((tert-butoxycarbonyl)oxy)-3-hydroxy-2-(4-(thiazol-5-yl)benzyl)pyrrolidine-1-carboxylate (Int-12, preparation in carbamate procedure) instead of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure), 3-[(2S)-1-(tert-butoxycarbonyl)pyrrolidin-2-yl]propanoic acid (5-4) in STEP 1; Boc Deprotection; General Procedure I in STEP 2. MS: m/z Calc'd for C₂₁H₂₇N₃O₃S [M+H]⁺402, found [M+H]⁺402. ¹H NMR (400 MHz, Methanol-d₄) δ 8.99 (s, 1H), 8.19 (s, 1H), 7.74-7.66 (m, 2H), 7.44 (d, J=8.0 Hz, 2H), 5.22-5.16 (m, 1H), 4.42 (d, J=4.3 Hz, 1H), 4.36-4.27 (m, 1H), 3.70-3.56 (m, 2H), 3.40-3.34 (m, 2H), 3.28-3.17 (m, 2H), 3.11 (dd, J=14.3, 8.9 Hz, 1H), 2.75-2.66 (m, 2H), 2.21-1.97 (m, 1H), 2.20-1.97 (m, 4H), 1.79-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Preparation of (2R,3S,4S)-4-hydroxy-2-{[4-(1,3-oxazol-5-yl)phenyl]methyl}pyrrolidin-3-yl 2-(3-fluorophenyl)acetate (V192843

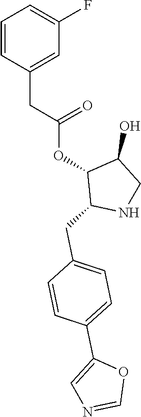 [SMILES: O=C(Cc1cccc(F)c1)O[C@@H]1[C@@H](O)CN[C@@H]1Cc1ccc(-c2cnco2)cc1]

The title compound was prepared in 15.7% overall yield as a white solid according to Condensation reaction with DCC, DMAP for synthesis of ester; General Procedure II using M-fluorophenylacetic acid in STEP 1; Boc Deprotection; General Procedure I in STEP 2. MS: m/z Calc'd for C₂₂H₂₁FN₂O₄[M+H]⁺397, found 397. ¹H NMR (400 MHz, Methanol-d₄) δ 8.27 (s, 1H), 7.75-7.67 (m, 2H), 7.53 (s, 1H), 7.47-7.40 (m, 1H), 7.28 (d, J=8.1 Hz, 2H), 7.19 (dd, J=15.3, 8.7 Hz, 2H), 7.12-7.05 (m, 1H), 5.10 (s, 1H), 4.37 (d, J=4.1 Hz, 1H), 4.36-4.25 (m, 1H), 3.86 (d, J=3.3 Hz, 2H), 3.57 (dd, J=11.9, 4.7 Hz, 1H), 3.26 (d, J=12.6 Hz, 1H), 3.16-2.97 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 17% B to 47% B in 10 min; Wave Length: 254 nm/220 nm; RT1(min): 8.5.

### Synthetic Preparation of (2R,3S,4S)-4-hydroxy-2-{[4-(1,3-thiazol-5-yl)phenyl]methyl}pyrrolidin-3-yl 2-(3-fluorophenyl)acetate (V192844

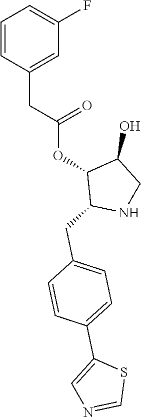 [SMILES: O=C(Cc1cccc(F)c1)O[C@@H]1[C@@H](O)CN[C@@H]1Cc1ccc(-c2cncs2)cc1]

The title compound was prepared in 31.4% overall yield as a white solid according to Condensation reaction with DCC, DMAP for synthesis of ester; General Procedure II using tert-butyl (2R,3S,4S)-4-((tert-butoxycarbonyl)oxy)-3-hydroxy-2-(4-(thiazol-5-yl)benzyl)pyrrolidine-1-carboxylate (Int-12, preparation in carbamate procedure) instead of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure), M-fluorophenylacetic acid in STEP 1; Boc Deprotection; General Procedure I in STEP 2. MS: m/z Calc'd for C₂₂H₂₁FN₂O₃S [M+H]⁺413, found 413. ¹H NMR (400 MHz, Methanol-d₄) δ 8.99 (d, J=0.8 Hz, 1H), 8.18 (d, J=0.7 Hz, 1H), 7.68-7.61 (m, 2H), 7.43 (dd, J=7.9, 6.0 Hz, 1H), 7.30-7.10 (m, 5H), 5.14-5.08 (m, 1H), 4.38 (d, J=4.1 Hz, 1H), 4.29 (dd, J=7.8, 3.4 Hz, 1H), 3.86 (d, J=2.8 Hz, 2H), 3.57 (dd, J=12.6, 4.3 Hz, 1H), 3.27 (d, J=12.6 Hz, 1H), 3.11 (dd, J=14.2, 7.2 Hz, 1H), 3.01 (dd, J=14.2, 8.5 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.5.

### Synthetic Preparation of (2R,3S,4S)-4-hydroxy-2-[(4-methoxyphenyl)methyl]pyrrolidin-3-yl 3-[(2S)-pyrrolidin-2-yl]propanoate (V192931

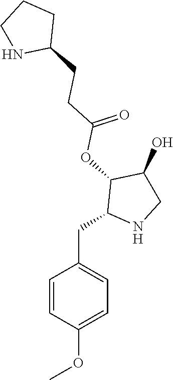 [SMILES: COc1ccc(C[C@H]2NC[C@H](O)[C@H]2OC(=O)CC[C@@H]2CCCN2)cc1]

The title compound was prepared in 36.5% overall yield as a white solid according to Condensation reaction with DCC, DMAP for synthesis of ester; General Procedure II using tert-butyl (2R,3S,4S)-4-[(tert-butoxycarbonyl)oxy]-3-hydroxy-2-[(4-methoxyphenyl)methyl]pyrrolidine-1-carboxylate (Int-1, preparation in carbamate procedure) instead of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure), 3-[(2S)-1-(tert-butoxycarbonyl)pyrrolidin-2-yl]propanoic acid (5-4) in STEP 1; Boc Deprotection; General Procedure I in STEP 2. MS: m/z Calc'd for C₁₉H₂₈N₂O₄ [M+H]⁺348, found 348. ¹H NMR (400 MHz, Methanol-d₄) δ 7.29-7.21 (m, 2H), 6.98-6.90 (m, 2H), 5.14 (d, J=3.4 Hz, 1H), 4.40 (d, J=4.3 Hz, 1H), 4.22 (m, 1H), 3.82-3.77 (s, 3H), 3.69-3.56 (m, 2H), 3.39-3.34 (m, 1H), 3.32-3.29 (m, 1H), 3.23 (d, J=12.7 Hz, 1H), 3.10 (dd, J=14.3, 6.6 Hz, 1H), 3.00 (dd, J=14.4, 8.9 Hz, 1H), 2.74-2.65 (m, 2H), 2.34-2.22 (m, 1H), 2.22-1.97 (m, 4H), 1.81-1.65 (m, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Preparation of (2R,3S,4S)-4-hydroxy-2-{[4-(1,3-oxazol-5-yl)phenyl]methyl}pyrrolidin-3-yl acetate V192987

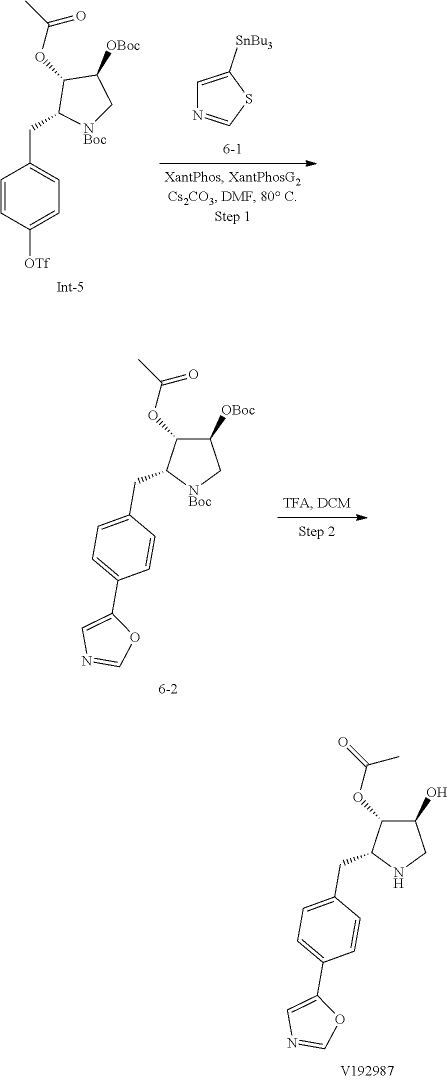

Step 1: To a stirred mixture of 5-(tributylstannyl)-1,3-thiazole (6-1, 512.9 mg, 1.37 mmol, 2 equiv) and tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, preparation in carbamate procedure, 400 mg, 0.685 mmol, 1.00 equiv) and CuCl (135.7 mg, 1.37 mmol, 2 equiv) in DMF (10 mL) was added XantPhos (158.6 mg, 0.27 mmol, 0.4 equiv) and XantPhos Pd G₂ (121.8 mg, 0.13 mmol, 0.2 equiv) at room temperature under nitrogen atmosphere. The resulting mixture was stirred at 80° C. for overnight under nitrogen atmosphere. The residue was purified by reversed-phase flash chromatography to afford tert-butyl (2R,3S,4S)-3-(acetyloxy)-4-[(tert-butoxycarbonyl)oxy]-2-{[4-(1,3-thiazol-5-yl)phenyl]methyl}pyrrolidine-1-carboxylate (300 mg, 84.3% yield, 90% purity) as a white solid. MS: m/z: Calc'd for C₂₆H₃₄N₂O₇S [M+H]⁺519, found 519.

Step 2: The title compound was prepared in 33.0% yield as a white solid according to Boc Deprotection; General Procedure I. MS: m/z Calc'd for C₁₆H₁₈N₂O₄[M+H]⁺303, found 303. ¹H NMR (400 MHz, Methanol-d₄) δ 8.27 (s, 1H), 7.77 (d, J=8.0 Hz, 2H), 7.55 (s, 1H), 7.45 (d, J=8.0 Hz, 2H), 5.13 (d, J=3.4 Hz, 1H), 4.39 (d, J=4.5 Hz, 1H), 4.32-4.23 (m, 1H), 3.68-3.59 (m, 1H), 3.28-3.17 (m, 2H), 3.09 (dd, J=14.3, 8.8 Hz, 1H), 2.21 (s, 3H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.5.

### Synthetic Preparation of (2R,3S,4S)-2-{[4-(difluoromethyl)phenyl]methyl}-4-hydroxypyrrolidin-3-yl 2-[(3S)-pyrrolidin-3-yl]acetate (V193046

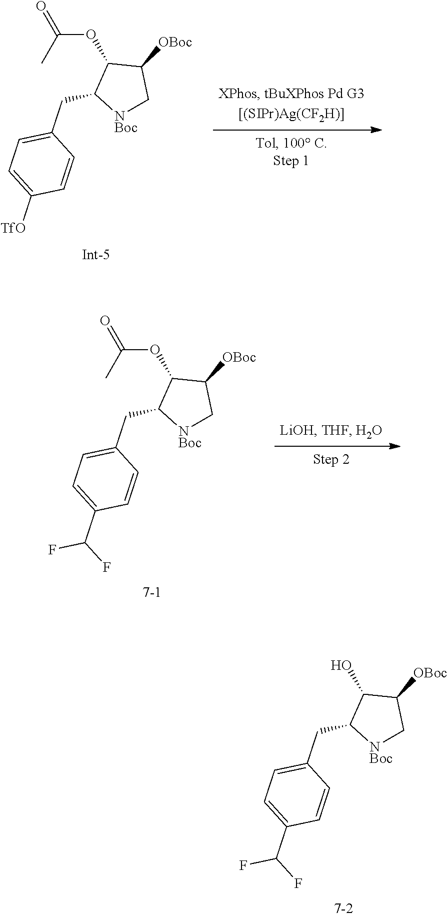

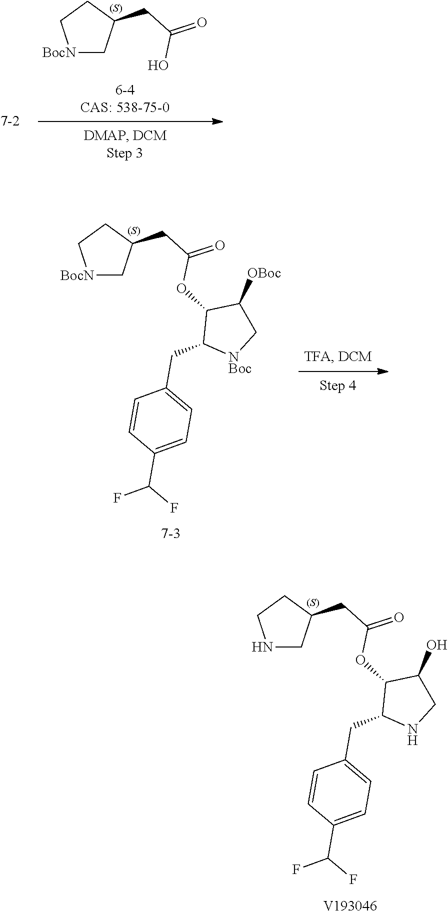

Step 1: To the solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 105 mg, 0.18 mmol, 1 equiv) and [1,3-bis(2,6-diisopropylphenyl)imidazolidin-2-ylidene](difluoromethyl)silver (129 mg, 0.23 mmol, 1.3 equiv) in toluene (5 mL) was added XPhos (9 mg, 0.018 mmol, 0.1 equiv) and t-BuXPhos Pd G3 (14 mg, 0.018 mmol, 0.1 equiv). The mixture was stirred at 100° C. for 7 hours under N₂. The mixture was diluted with EA, washed with water, dried and concentrated to give the crude product used directly in the next step. The tert-butyl (2R,3S,4S)-3-(acetyloxy)-4-[(tert-butoxycarbonyl)oxy]-2-{[4-(difluoromethyl)phenyl]methyl}pyrrolidine-1-carboxylate (80 mg, 91.6% yield) was obtained as a yellow oil. MS: m/z: Calc'd for C₂₄H₃₃F₂NO₇ [M+H−56−56]⁺373, found 373.

Step 2: To a stirred solution of tert-butyl (2R,3S,4S)-3-(acetyloxy)-4-[(tert-butoxycarbonyl)oxy]-2-{[4-(difluoromethyl)phenyl]methyl}pyrrolidine-1-carboxylate (7-1, 100 mg, 0.21 mmol, 1 equiv) in THF (1 mL) was added LiOH (15.1 mg, 0.63 mmol, 3 equiv) in H₂O (0.5 mL) and EtOH (0.5 mL) at room temperature. The resulting mixture was stirred at room temperature for 4 h. After completion of reaction monitored by LCMS. Concentrated, the residue was purified by flash to afford tert-butyl (2R,3S,4S)-4-((tert-butoxycarbonyl)oxy)-2-(4-(difluoromethyl)benzyl)-3-hydroxypyrrolidine-1-carboxylate (79.8 mg, 87.6% yield, 98% purity) as a yellow oil. MS: m/z Calc'd for C₂₂H₃₁F₂NO₆ [M+H]⁺444, found 444.

### Step 3 and 4

The title compound was prepared in 45.4% overall yield as a white solid according to Condensation reaction with DCC, DMAP for synthesis of ester; General Procedure II using tert-butyl (2R,3S,4S)-4-((tert-butoxycarbonyl)oxy)-2-(4-(difluoromethyl)benzyl)-3-hydroxypyrrolidine-1-carboxylate (7-2) instead of t-butyl (2R,3S,4S)-4-[(tert-butoxycarbonyl)oxy]-3-hydroxy-2-{[4-(1,3-oxazol-5-yl)phenyl]methyl}pyrrolidine-1-carboxylate (Int-9, preparation in carbamate procedure), (S)-2-(1-(tert-butoxycarbonyl)pyrrolidin-3-yl)acetic acid in STEP 1; Boc Deprotection; General Procedure I in STEP 2. MS: m/z Calc'd for C₁₈H₂₄F₂N₂O₃[M+H]⁺355; Found, 355. ¹H NMR (400 MHz, Methanol-d₄) δ 7.58 (d, J=7.9 Hz, 2H), 7.49 (d, J=8.0 Hz, 2H), 6.94-6.60 (m, 1H), 5.17 (d, J=3.8 Hz, 1H), 4.43-4.37 (m, 1H), 4.35-4.25 (m, 1H), 3.70-3.54 (m, 2H), 3.50-3.39 (m, 1H), 3.31-3.27 (m, 1H), 3.25 (d, J=5.4 Hz, 1H), 3.22 (d, J=6.7 Hz, 1H), 3.15 (dd, J=14.3, 9.0 Hz, 1H), 3.06-2.93 (m, 1H), 2.88-2.65 (m, 3H), 2.39-2.26 (m, 1H), 1.81-1.67 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.5.

### Synthetic Preparation of (2R,3S,4S)-4-hydroxy-2-{[4-(1,3-oxazol-5-yl)phenyl]methyl}pyrrolidin-3-yl 2-[(3S)-pyrrolidin-3-yl]acetate (V193047

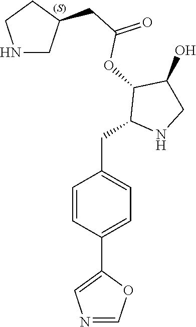

The title compound was prepared in 36.1% overall yield as a white solid according to Condensation reaction with DCC, DMAP for synthesis of ester; General Procedure II using [(3S)-1-(tert-butoxycarbonyl)pyrrolidin-3-yl]acetic acid in STEP 1; Boc Deprotection; General Procedure I in STEP 2. MS: m/z Calc'd for C₂₀H₂₅N₃O₄[M+H]⁺372, found 372. ¹H NMR (400 MHz, Methanol-d₄) δ 8.28 (s, 1H), 7.77 (d, J=8.2 Hz, 2H), 7.55 (s, 1H), 7.46 (d, J=8.3 Hz, 2H), 5.22-5.17 (m, 1H), 4.40 (d, J=4.3 Hz, 1H), 4.31 (dd, J=9.5, 6.5 Hz, 1H), 3.68-3.57 (m, 2H), 3.44 (dd, J=12.3, 8.5 Hz, 1H), 3.31-3.17 (m, 3H), 3.11 (dd, J=14.4, 9.0 Hz, 1H), 2.98 (dd, J=11.6, 8.4 Hz, 1H), 2.89-2.67 (m, 3H), 2.34 (dd, J=11.7, 5.7 Hz, 1H), 1.75 (dd, J=12.8, 8.6 Hz, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 19% B in 10 min; Wave Length: 254 nm/220 nm; RT1(min): 8.5.

### Additional Compounds

The following compounds can be synthesized accordingly to the above procedures:

| | |
|---|---|
| 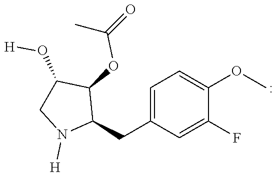 [SMILES: COc1ccc(C[C@H]2NC[C@H](O)[C@H]2OC(C)=O)cc1F] | V187569 |
| 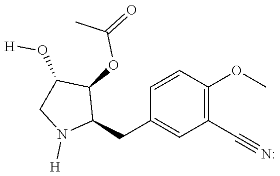 | V188045 |
| 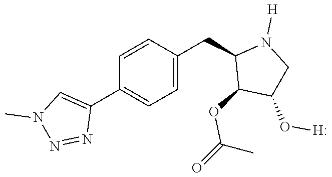 | V187748 |
| 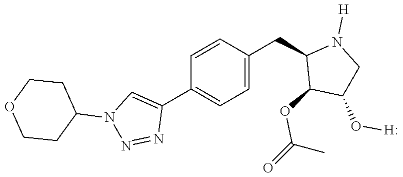 | V187838 |
| 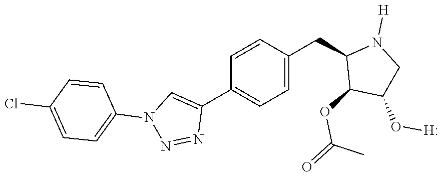 | V187839 |
| 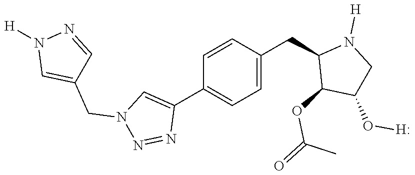 | V187844 |
| 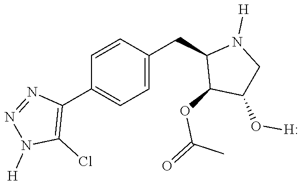 | V187854 |
| 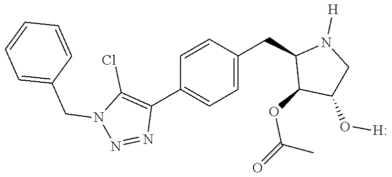 | V187856 |

### Example 4: In Vitro Transcription Translation Data and Cell Viability Data In Vitro Transcription/Translation Data

All reporter constructs have been cloned into the pT7CFE1-CHis plasmid (ThermoFisher #88860) using Gibson assembly procedures (NEB #E2611L). The parental pT7CFE1-CHis plasmid contains an EMCV IRES at the 5′UTR for translation initiation, a multiple-cloning site (MCS) for gene insertion, an optional C-terminal His-epitope tag, and polyA sequence in the 3′UTR to regulate mRNA stability. All reporters are designed to be inserted at the MCS to include a red fluorescent protein (mCherry), P2A self-cleaving peptide which releases the preceding mCherry protein product, and a fusion open reading frame containing a 3×FLAG epitope, a peptide sequence of interest (TEST), and green fluorescent protein (sfGFP).

Construct Plasmid Preparation: All plasmids are isolated from MACH1 cells (ThermoFisher) grown overnight in ampicillin-containing luria broth, and purified using ZymoPure Express Plasmid Midiprep Kit (Zymo Research). Constructs are sequenced verified and diluted to a working concentration of 110 ng/μL.

HeLa S3 Lysate Preparation: HeLa S3 cell pellets are acquired from Ipracell/Ipratech (Belgium) and shipped as 4 mL frozen cell pellets on dry ice for storage at −80° C. until use. The procedure for lysate preparation is as follows. Prepare 1× Lysis Buffer, cold and stored on ice:

| Hela S3 Lysis Buffer (1X) | Vol. for 40 mL of buffer |
|---|---|
| 20 mM Hepes pH 7.4 | 800 μL of 1M stock |
| 10 mM KOAc pH 7.6 | 133.2 μL of 3M stock |
| 1.8 mM Mg(OAc)₂ | 72 μL of 1M stock |
| 1 mM DTT | 40 μL of 1M stock |
| 500 nM ISRIB | 20 μL of 1 mM stock |
| H₂O (cold nuclease free) | 38.936 |

Each preparation takes 8 cell pellets (4 ml packed volume each, approximately 1.0×10⁹ cells per pellet). Thaw cell pellets from −80° C. on ice for 90 mins. Lay pellets horizontally on top of the ice and rotate every 30 mins to ensure even thawing. Once thawed, add 4 mL of cold lysis buffer to each pellet, and using a serological pipet, gently pipet up and down to break up clumps. Incubate resuspended cells in ice for an additional 30 mins (plunged into ice). During 30 min incubation, rinse and RNase-zap (Ambion) glass dounces (use loose pestle “A”), gently dry and place on ice until needed. Prepare RNase-free Eppendorf tubes for spinning and for batch storage. Add batch labels to 1.5-1.7 mL RNase-free microcentrifuge tubes (labeling scheme below). Each batch will have an extra 1.5-1.7 mL RNase-free microcentrifuge tube for 40 μL of lysate, label the top of this tube (labeling scheme below). After 30 min incubation, transfer all 8 mL of cell pellet to dounce. Cell lysis is complete after douncing 100 times. Aliquot cell lysis into 1.5-1.7 mL temporary-microcentrifuge tubes. Place tubes in centrifuge with “fins” out, and spin at 1,200 xg for 5 mins. After the first spin use a 1 mL pipet to resuspend each tube and repeat spin. This acts as an additional lysis step, is more vigorous and increases lysate yield. During second spin get liquid N₂ and a fresh conical tube on ice.

After spin is complete, transfer supernatant (lysate) from microcentrifuge tubes into the 50 mL conical tube and keep on ice. Repeat steps 10-14 for all other cell pellets until completed. Once all cell lysate has been transferred to the 50 mL conical tube pipet to mix so the solution is homogenous. Transfer 1 mL of lysate into batch-labeled microcentrifuge tubes and flash freeze in liquid nitrogen. Be sure to pipet up and down to mix throughout transfer step. Place all tubes in the 80° C. for long-term storage.

Lysate batch quality control: Use IDB-PL005 & IDB-PL055 reporters (highest & lowest signal generators) as controls. Reaction Set-Up included 5 μL Lysate (Previous preparation or new preparation), 1 μL TF accessory proteins, 2 μL TF reaction mix, 1 μL H₂O, and 1 μL of 110 ng/μL plasmid DNA. Set-up 96-well plate as seen below, and incubate reactions at 30° C. for 2 hours:

| | Control lysate reactions | | | Batch #00X Rxns | | |
|---|---|---|---|---|---|---|
| | 1 | 2 | 3 | 4 | 5 | 6 |
| A | Lysate | PL005 | PL055 | Lysate | PL005 | PL055 |
| | Only | | | Only | | |

After 2 hrs, quench the assay using 45 μL of PBS, and transfer 50 μL to a flat-bottom black microplate. Compare fluorescent signals between previous lysate batch and new batch to confirm activity.

In vitro transcription-translation assay setup: Each assay includes a master mix containing lysate and shared components, a 96-well plate containing compound titrations for testing, a 96-well DNA plate with reporter constructs to be tested, 96-well skirted PCR plates, and 96-well flat-bottom, black, fluorescent assay plates. Reaction materials setup: (A) Master mix—15 mL conical tube (3 mL of HeLa S3 Lysate described above; 0.6 mL of ThermoFisher Accessory Proteins (#88882); 1.2 ml of ThermoFisher Reaction Mix (#88882); 0.54 mL of nuclease-free Water); (B) 96-Well Master Mix+Compound Plate (Half-log dilutions (30 μM-0.1 μM) of 4 compounds of interest are prepared in 100% DMSO); 96-Well DNA Plate (Five DNA constructs are plated into single columns within the plate. Row 1 of the column contains nuclease free H₂O, all other wells contain 110 ng/μL plasmid DNA). Reactions are started by mixing 155 μL of master mix and 3.5 μL of compound serial dilutions, then 9.1 μL of master mix+compound mixture is aliquoted across 5 96-well plates. Reactions begin with final addition of 2 μL of DNA for a 11.1 μL reaction volume in each well. The entire plate is sealed, and incubated for 2 hrs at 30° C. After 2 hrs, quench the assay using 45 μL of PBS, and transfer 50 μL to a flat-bottom black microplate Quantify the sfGFP and mCherry signals from each well on each plate, taking 3 reads of each plate, and perform data analysis below.

In vitro transcription-translation assay data analysis: Each plate is read three times in channels for both sfGFP and mCherry signals. The average signal across these 3 reads is then calculated for further analysis. Control wells containing no DNA reporter are subtracted from reporter wells in the same column to remove auto-fluorescence of the lysate. Then, the ratio of sfGFP/mCherry is calculated per well, and these ratios are averaged across the triplicate experiments on the same plate. Finally, the ratio of each titration concentration is normalized to the no drug control to create the normalized stalling metric. The error on this measurement is determined by propagating error through the averaging and normalization steps. Values are plotted and fit for IC₅₀ determination for each test sequence and titration of compound.

TABLE E2
In Vitro Transcription-Translation *

| Com- | IVTT ALK | IVTT MYC | IVTT KRAS |
|---|---|---|---|
| pound | IC₅₀ (μM) | IC₅₀ (μM) | G12D IC₅₀ (μM) |
| V186777 | A | A | A |
| V187049 | A | A | A |
| V187010 | A | A | A |
| V186812 | — | A | A |
| V187048 | A | A | A |
| V0112 | A | A | D |
| V186990 | A | A | A |
| V187056 | A | A | — |
| V000144 | A | A | D |
| V187054 | — | A | A |
| V187006 | A | A | A |
| V187043 | A | A | A |
| V0109 | A | A | A |
| V187168 | A | A | — |
| V0110 | — | A | A |
| V186940 | A | A | A |
| V187009 | A | A | A |
| V187082 | A | A | A |
| V0113 | A | A | A |
| V186810 | A | A | A |
| V187173 | A | A | A |
| V187035 | A | A | A |
| V187731 | A | A | A |
| V186825 | A | A | A |
| V187050 | A | A | — |
| V187169 | — | A | A |
| V187368 | A | A | A |
| V186859 | A | A | A |
| V187052 | A | A | — |
| V187176 | — | A | A |
| V187177 | A | A | A |
| V186783 | A | A | A |
| V186809 | A | A | A |
| V187063 | A | A | A |
| V186761 | A | A | D |
| V187720 | A | A | A |
| V187057 | A | A | A |
| V187175 | A | A | A |
| V0111 | A | A | A |
| V186921 | — | A | A |
| V187069 | A | A | — |
| V186779 | A | A | A |
| V187061 | A | A | A |
| V187690 | A | A | A |
| V187053 | A | A | A |
| V187045 | A | — | — |
| V187088 | A | A | A |
| V187172 | A | A | A |
| V187750 | A | A | A |
| V187162 | A | A | A |
| V187166 | A | A | A |
| V187070 | — | A | A |
| V187096 | A | A | — |
| V187689 | A | A | A |
| V000215 | A | A | A |
| V186775 | A | A | C |
| V186873 | A | A | A |
| V186865 | A | A | D |
| V187055 | — | A | A |
| V187072 | A | A | A |
| V187170 | A | A | — |
| V187171 | A | — | A |
| V187178 | A | A | A |
| V187179 | A | A | A |
| V187184 | A | A | A |
| V186760 | A | A | C |
| V186992 | A | A | — |
| V187067 | A | — | B |
| V186862 | A | A | D |
| V187163 | A | A | A |
| V187164 | — | A | A |
| V187134 | A | A | — |
| V187517 | A | — | C |
| V187533 | A | B | D |
| V186765 | — | A | B |
| V0106 | A | B | C |
| V187008 | A | A | — |
| V186870 | A | A | B |
| V186824 | A | D | D |
| V186864 | A | B | D |
| V186857 | A | D | D |
| V186871 | — | C | D |
| V186774 | — | C | D |
| V187181 | A | C | — |
| V187123 | A | B | D |
| V186784 | A | A | B |
| V186866 | A | B | D |
| V187028 | A | D | D |
| V187694 | A | A | B |
| V187044 | A | B | D |
| V186849 | A | B | D |
| V186785 | A | D | D |
| V0108 | — | D | D |
| V187155 | B | D | D |
| V187066 | B | D | D |
| V187159 | B | B | D |
| V187182 | B | D | — |
| V186822 | — | — | D |
| V0107 | C | D | D |
| V186823 | C | D | D |
| V186850 | C | D | D |
| V0099 | D | D | D |
| V0081 | D | D | D |
| V0092 | D | D | D |
| V0102 | D | D | D |
| V000148 | D | D | D |
| V000173 | D | D | D |
| V041828 | D | D | — |
| V000192 | D | D | D |
| V000196 | D | D | — |
| V000198 | D | D | D |
| V186712 | D | D | D |
| V186717 | D | D | — |
| V186718 | D | — | D |
| V186763 | D | — | D |
| V186772 | D | D | D |
| V186780 | — | — | D |
| V186781 | D | D | D |
| V186782 | D | D | — |
| V186854 | D | D | — |
| V186858 | D | D | D |
| V186868 | D | — | D |
| V186989 | D | D | D |
| V187030 | D | D | D |
| V187187 | D | — | D |
| V187324 | D | C | D |
| V186811 | — | A | A |
| V187263 | — | D | — |
| V187569 | — | A | — |
| V187748 | — | A | — |
| V187751 | — | A | — |
| V187838 | — | A | — |
| V187839 | — | A | — |
| V187840 | — | A | — |
| V187844 | — | A | — |
| V187854 | — | A | — |
| V187855 | — | A | — |
| V187856 | — | A | — |
| V188005 | — | D | — |
| V188045 | — | D | — |
| V188046 | — | A | — |
| V188057 | — | A | — |
| V188168 | — | A | — |
| V192387 | — | A | — |
| V192840 | — | A | — |
| V192841 | — | A | — |
| V192842 | — | A | — |
| V192843 | — | A | — |
| V192844 | — | A | — |
| V192931 | — | A | — |
| V192987 | — | A | — |
| V193046 | — | A | — |
| V193047 | — | A | — |
| V188182 | — | D | — |
| V187185 | — | A | — |

* A= 0 to <10; B = 10 to <20; C = 20 to <30; D = >30; Compounds not tested in any of the three IVTT assays were excluded from Table E2

TABLE E3
In Vitro Transcription-Translation *

| Compound | BCL-2 IC₅₀ (μM) | CCND1 IC₅₀ (μM) | MCL-1 IC₅₀ (μM) |
|---|---|---|---|
| V187690 | — | A | A |
| V186777 | — | A | — |
| V187049 | — | A | — |
| V186812 | — | A | — |
| V0112 | — | A | — |
| V187056 | — | A | — |
| V0109 | — | A | — |
| V187168 | — | A | — |
| V187082 | — | A | — |
| V187035 | — | A | A |
| V187176 | — | A | — |
| V186921 | — | A | — |
| V187690 | — | A | — |
| V187045 | — | A | — |
| V187088 | — | A | — |
| V187172 | — | A | — |
| V187750 | — | A | — |
| V187162 | — | A | — |
| V187055 | — | A | A |
| V187170 | — | A | — |
| V186862 | — | D | — |
| V187155 | — | A | — |
| V187690 | A | A | A |
| V187070 | — | A | A |
| V187263 | C | C | C |
| V187569 | — | A | A |
| V187748 | A | A | A |
| V187751 | A | A | A |
| V187838 | A | A | A |
| V187839 | A | A | A |
| V187840 | A | A | A |
| V187844 | A | A | A |
| V187854 | A | A | A |
| V187855 | A | A | A |
| V187856 | A | A | A |
| V188005 | D | D | D |
| V188045 | — | D | D |
| V188046 | — | A | A |
| V188182 | — | — | D |
| V188057 | — | A | — |
| V188168 | — | A | A |

* A= 0 to <10; B = 10 to <20; C = 20 to <30; D = >30; Compounds not tested in any of the three IVTT assays were excluded from Table E3

### Cell Viability Data

Count and dispense 2,000-10,000 cells per well (100 μL volume) in black 96-well tissue culture microplate and let cells adhere overnight. Add compound dissolved in 100% DMSO to each well as needed and incubate cells in standard culture conditions for 24-72 hrs. Final DMSO concentration in each well should be 0.1-0.5%. At the desired time point, gently aspirate compound containing media and replace with PBS to wash. Repeat wash step for 2 total washes. Remove PBS and add 100 μL CellTiter-Fluor reagent (Promega) reagent to each well. Seal plate and incubate 1 hr at room temperature with orbital shaking. Analyze fluorescence using plate reader to quantify viable cells compared with vehicle (DMSO) control.

TABLE E3
SU Cell Viability Data (HT) *

| | HEK | | | | | NCI- | | | | | |
|---|---|---|---|---|---|---|---|---|---|---|---|
| | 293T | HPAF- | KLE | LS411N | MCF7 | H1915 | HCC38 | HEPG2 | KATO- | MS751 | T47D |
| | IC₅₀ | II IC₅₀ | IC₅₀ | IC₅₀ | IC₅₀ | IC₅₀ | IC₅₀ | IC₅₀ | III IC₅₀ | IC₅₀ | IC₅₀ |
| Cmpd | (μM) | (μM) | (μM) | (μM) | (μM) | (μM) | (μM) | (μM) | (μM) | (μM) | (μM) |
| V0109 | A | B | B | B | B | B | — | — | — | — | — |
| V187043 | A | B | A | A | A | B | A | — | B | A | A |
| V187049 | A | A | A | A | A | A | A | — | A | A | A |
| V187061 | A | B | B | A | B | B | B | — | C | A | B |
| V187690 | B | D | C | B | B | D | C | B | C | B | C |

* A = 0 to <0.25; B = 0.25 to <1; C = 1 to <4; D = ≥4; Compounds not tested in any of the cell viability assays were excluded from Table E3

### Incorporation by Reference

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the Figures, the Examples, and the Claims.

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I): 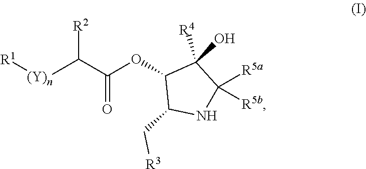
or a pharmaceutically acceptable salt thereof, wherein:
R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂;
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring;
R² is hydrogen or optionally substituted alkyl;
or R¹ and R² are joined together with their intervening atoms to form optionally substituted carbocyclyl or optionally substituted heterocyclyl;
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl;
each instance of Y is independently —C(R^{Y})₂—, —O—, —N(R^{1a})—, or —S—;
each instance of R^{Y} is independently hydrogen or halogen, or two instances of R^{Y} are taken together to form ═O; and
n is 0, 1, 2, 3, 4, or 5.

2. The compound of claim 1, wherein the compound is of Formula (II): 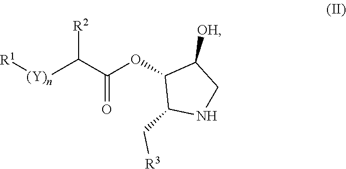
or a pharmaceutically acceptable salt thereof.

3. The compound of any one of claims 1 or 2, or a pharmaceutically acceptable salt thereof, wherein R¹ is hydrogen, optionally substituted alkyl, optionally substituted alkenyl, or optionally substituted alkynyl.

4. The compound of any one of claims 1-3, or a pharmaceutically acceptable salt thereof, wherein R¹ is hydrogen, methyl, ethyl, isopropyl, 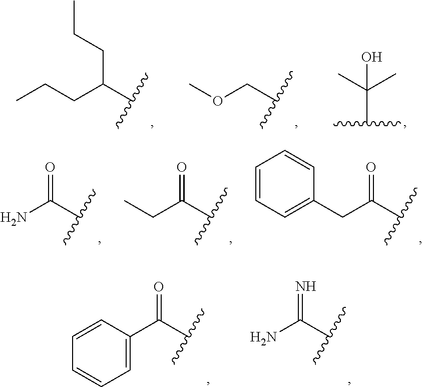
—CF₃, or ethynyl.

5. The compound of any one of claims 1-4, or a pharmaceutically acceptable salt thereof, wherein R¹ is hydrogen, methyl, ethyl, isopropyl, 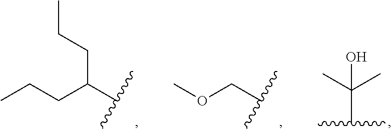
or —CF₃.

6. The compound of any one of claims 1 or 2, or a pharmaceutically acceptable salt thereof, wherein R¹ is optionally substituted carbocyclyl, optionally substituted heterocyclyl, optionally substituted aryl, or optionally substituted heteroaryl.

7. The compound of any one of claims 1, 2, or 6, or a pharmaceutically acceptable salt thereof, wherein R¹ is optionally substituted carbocyclyl.

8. The compound of any one of claims 1, 2, 6, or 7, or a pharmaceutically acceptable salt thereof, wherein R¹ is optionally substituted cyclopropyl, optionally substituted cyclobutyl, optionally substituted cyclohexyl, or optionally substituted cyclobutenyl.

9. The compound of any one of claims 1, 2, or 6-8, or a pharmaceutically acceptable salt thereof, wherein R¹ is

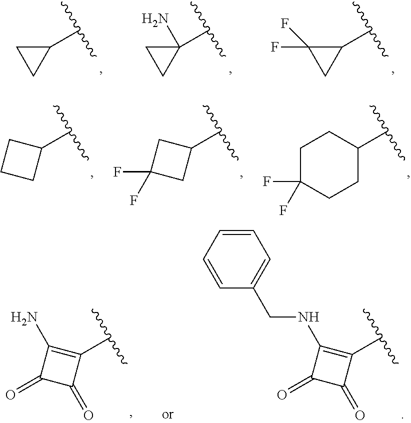

10. The compound of any one of claims 1, 2, or 6, or a pharmaceutically acceptable salt thereof, wherein R¹ is optionally substituted heterocyclyl.

11. The compound of any one of claims 1, 2, 6, or 10, or a pharmaceutically acceptable salt thereof, wherein R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydroimidazopyrazinyl, optionally substituted tetrahydrothiopyranyl, optionally substituted 2,6-diazaspiro[3.3]heptyl, optionally substituted 6-azaspiro[3.4]octyl, 2,6-diazaspiro[3.4]octyl, optionally substituted 2,5,8-triazaspiro[3.5]nonyl, or optionally substituted 1,4,9-triazaspiro[5.5]undecyl.

12. The compound of any one of claims 1, 2, 6, 10, or 11, or a pharmaceutically acceptable salt thereof, wherein R¹ is optionally substituted tetrahydrofuranyl, optionally substituted azetidinyl, optionally substituted pyrrolidinyl, optionally substituted imidazolidinyl, optionally substituted piperazinyl, optionally substituted tetrahydroimidazopyrazinyl, or optionally substituted 2,5,8-triazaspiro[3.5]nonyl.

13. The compound of any one of claims 1, 2, 6, or 10-12, or a pharmaceutically acceptable salt thereof, wherein R¹ is

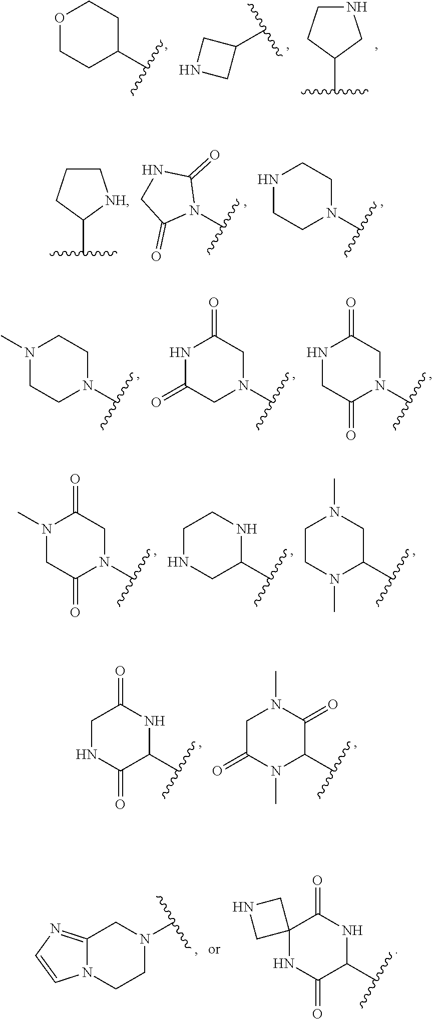

14. The compound of any one of claims 1, 2, or 6, or a pharmaceutically acceptable salt thereof, wherein R¹ is optionally substituted aryl.

15. The compound of any one of claims 1, 2, 6, or 14, or a pharmaceutically acceptable salt thereof, wherein R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂.

16. The compound of any one of claims 1, 2, 6, 14, or 15, or a pharmaceutically acceptable salt thereof, wherein R¹ is

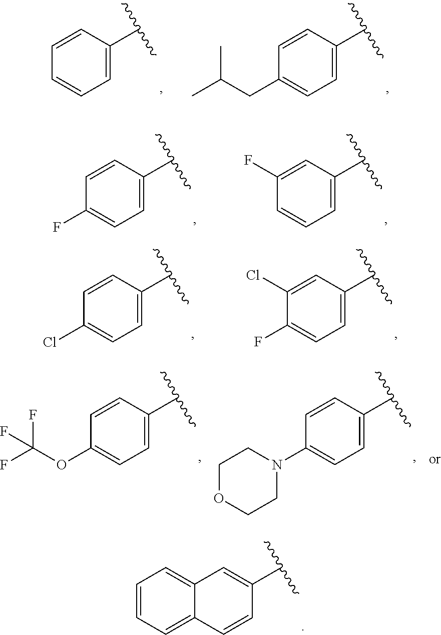

17. The compound of any one of claims 1, 2, or 6, or a pharmaceutically acceptable salt thereof, wherein R¹ is optionally substituted heteroaryl.

18. The compound of any one of claims 1, 2, 6, or 17, or a pharmaceutically acceptable salt thereof, wherein R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted benzotriazolyl, optionally substituted purinyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl.

19. The compound of any one of claims 1, 2, 6, 17, or 18, or a pharmaceutically acceptable salt thereof, wherein R¹ is optionally substituted pyridyl, optionally substituted pyrazinyl, optionally substituted pyrimidinyl, optionally substituted pyrazolyl, optionally substituted imidazolyl, optionally substituted triazolyl, optionally substituted tetrazolyl, optionally substituted benzotriazolyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl.

20. The compound of any one of claims 1, 2, 6, or 17-19, or a pharmaceutically acceptable salt thereof, wherein R¹ is

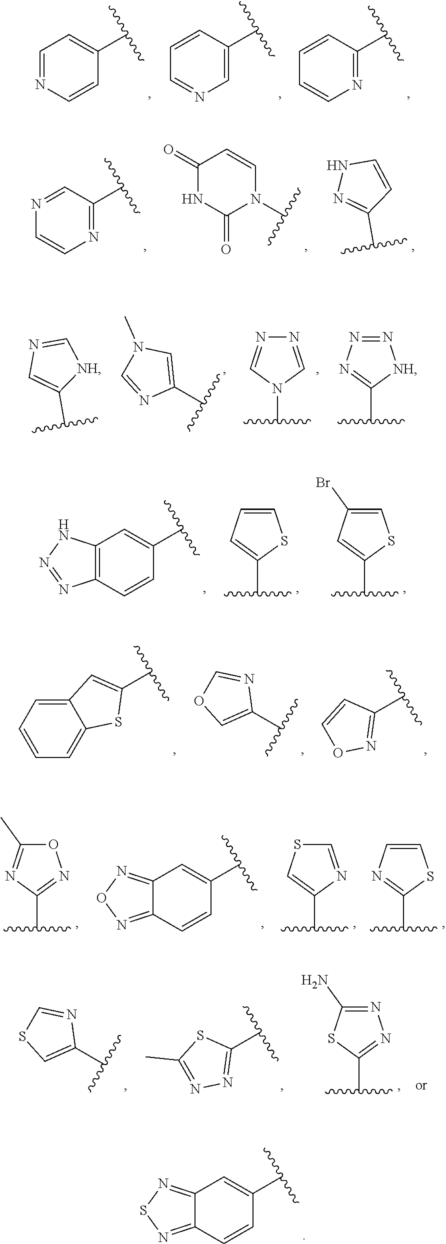

21. The compound of any one of claims 1 or 2, or a pharmaceutically acceptable salt thereof, wherein R¹ is —OR^{1a} or —N(R^{1a})₂.

22. The compound of any one of claims 1, 2 or 21, or a pharmaceutically acceptable salt thereof, wherein R¹ is —OH,

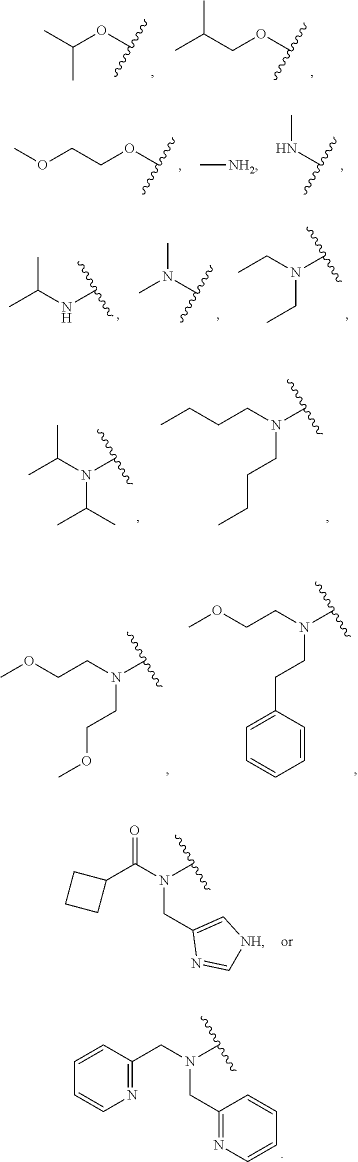

23. The compound of any one of claims 1, 2, 21, or 22, or a pharmaceutically acceptable salt thereof, wherein R¹ is —OH,

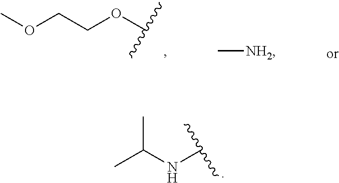

24. The compound of any one of claims 1-23, or a pharmaceutically acceptable salt thereof, wherein R² is hydrogen.

25. The compound of any one of claims 1-24, or a pharmaceutically acceptable salt thereof, wherein n is 0.

26. The compound of any one of claims 1-24, or a pharmaceutically acceptable salt thereof, wherein n is 1.

27. The compound of any one of claims 1-26, wherein the compound is of Formula (II-a-1) or (II-a-2): 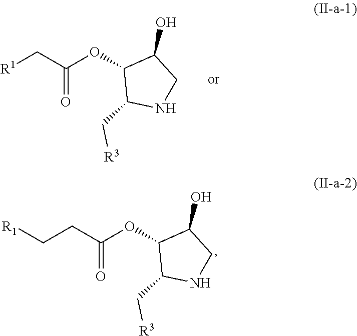
or a pharmaceutically acceptable salt thereof.

28. The compound of any one of claims 1-27, or a pharmaceutically acceptable salt thereof, wherein at least one instance of Y is C(R^{Y})₂.

29. The compound of any one of claims 1-28, or a pharmaceutically acceptable salt thereof, wherein at least one instance of Y is CH₂.

30. The compound of any one of claims 1-26, or a pharmaceutically acceptable salt thereof, wherein at least one instance of Y is —O— or —N(R^{1a}).

31. The compound of any one of claims 1, 2, 25, 26, or 28-30, or a pharmaceutically acceptable salt thereof, wherein R¹ and R² are joined together with their intervening atoms to form optionally substituted carbocyclyl.

32. The compound of any one of claims 1, 2, 25, 26, or 28-31, or a pharmaceutically acceptable salt thereof, wherein R¹ and R² are joined together with their intervening atoms to form optionally substituted cyclobutyl, optionally substituted cyclohexyl, optionally substituted spiro[2.2]pentyl, optionally substituted spiro[2.3]hexyl, or optionally substituted spiro[3.3]heptyl.

33. The compound of any one of claims 1, 2, 25, 26, or 28-32, or a pharmaceutically acceptable salt thereof, wherein R¹ and R² are joined together with their intervening atoms to form

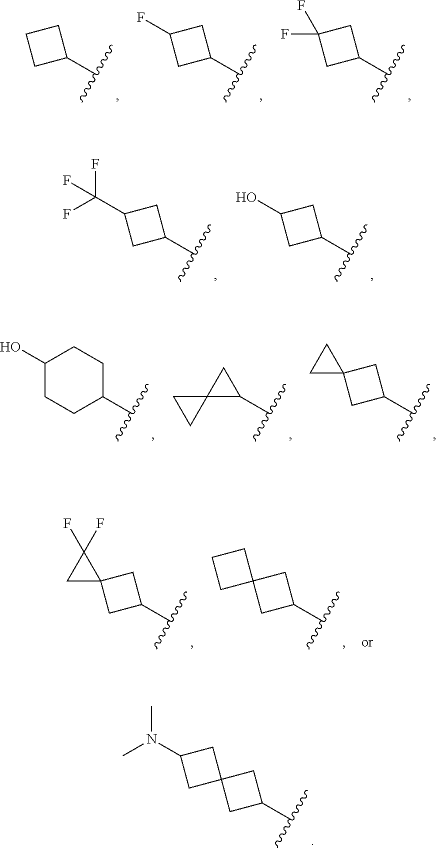

34. The compound of any one of claims 1, 2, 25, 26, or 28-30, or a pharmaceutically acceptable salt thereof, wherein R¹ and R² are joined together with their intervening atoms to form optionally substituted heterocyclyl.

35. The compound of any one of claims 1, 2, 25, 26, 28-30, or 34, or a pharmaceutically acceptable salt thereof, wherein R¹ and R² are joined together with their intervening atoms to form optionally substituted oxetanyl, optionally substituted tetrahydrofuranyl, optionally substituted tetrahydropyranyl, optionally substituted azetidinyl, optionally substituted pyrrolidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl.

36. The compound of any one of claims 1, 2, 25, 26, 28-30, 34, or 35, or a pharmaceutically acceptable salt thereof, wherein R¹ and R² are joined together with their intervening atoms to form optionally substituted oxetanyl, optionally substituted tetrahydrofuranyl, optionally substituted tetrahydropyranyl, optionally substituted azetidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl.

37. The compound of any one of claims 1, 2, 25, 26, 28-30, or 34-36, or a pharmaceutically acceptable salt thereof, wherein R¹ and R² are joined together with their intervening atoms to form

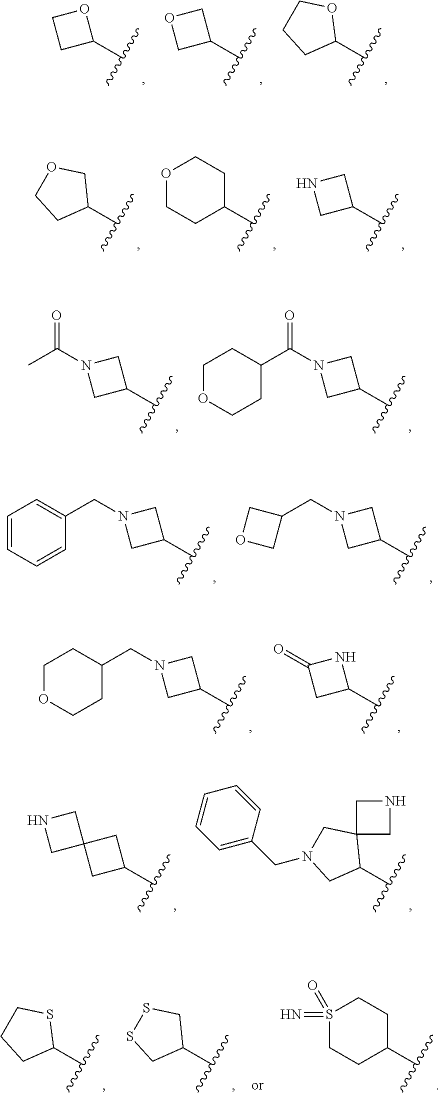

38. The compound of any one of claims 1-37, or a pharmaceutically acceptable salt thereof, wherein R³ is optionally substituted aryl.

39. The compound of any one of claims 1-38, or a pharmaceutically acceptable salt thereof, wherein R³ is phenyl optionally substituted with halogen, optionally substituted alkyl, optionally substituted alkynyl, —CN, —NO₂, —OR^{1a}, optionally substituted phenyl, optionally substituted triazolyl, optionally substituted tetrazolyl, optionally substituted thiazolyl, or optionally substituted oxazolyl.

40. The compound of any one of claims 1-37, wherein the compound is of Formula (III): 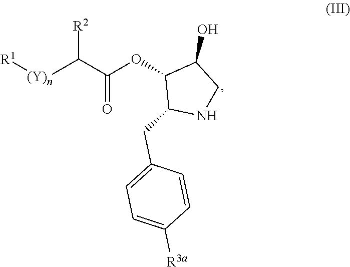
or a pharmaceutically acceptable salt thereof, wherein:
R^{3a} is halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring.

41. The compound of claim 40, wherein the compound is of Formula (III-b-1) or (III-b-2): 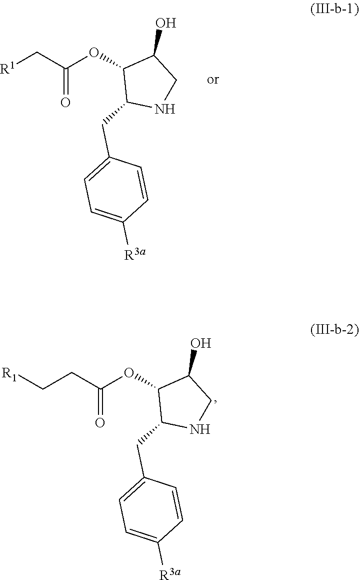
or a pharmaceutically acceptable salt thereof.

42. The compound of any one of claims 40 or 41, wherein R³, is halogen, optionally substituted alkyl, optionally substituted alkynyl, —CN, —NO₂, —OR^{1a}, optionally substituted phenyl, optionally substituted triazolyl, optionally substituted tetrazolyl, optionally substituted thiazolyl, or optionally substituted oxazolyl.

43. The compound of any one of claims 1-42, or a pharmaceutically acceptable salt thereof, wherein

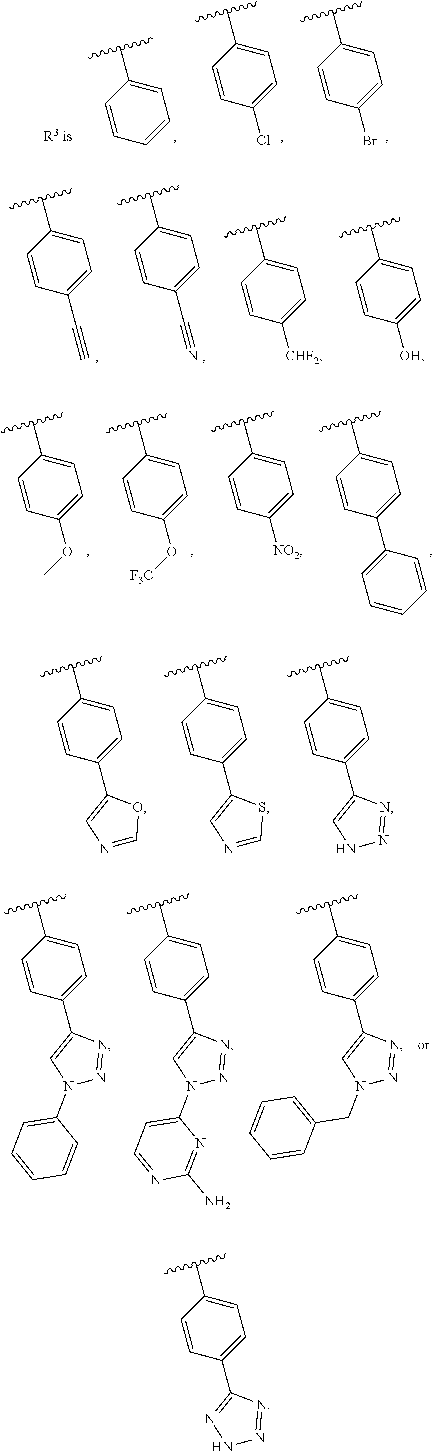

44. The compound of any one of claims 1-43, wherein R⁴ is hydrogen or methyl.

45. The compound of any one of claims 1-44, wherein the compound is of Formula (I-b-1): 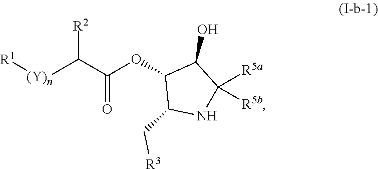
or a pharmaceutically acceptable salt thereof.

46. The compound of any one of claims 1-45, wherein R^{5a} and R^{5b} are each hydrogen, or R^{5a} and R^{5b} are joined together with their intervening atom to form optionally substituted cyclopropyl.

47. The compound of any one of claims 1-46, wherein the compound is of Formula (I-c-1): 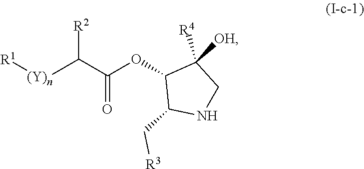
or a pharmaceutically acceptable salt thereof.

48. The compound of any one of claims 1-48, wherein the compound is selected from those in Table 1A and Table 1B, and pharmaceutically acceptable salts thereof.

49. A composition comprising the compound of any one of claims 1-48, or a pharmaceutically acceptable salt thereof, and an excipient.

50. A method of modulating protein synthesis in a subject in need thereof or in a cell, tissue, or biological sample, comprising administering to the subject in need thereof or contacting the cell, tissue, or biological sample with an effective amount of:
a compound of any one of claims 1-48, or a pharmaceutically acceptable salt thereof;
or the composition of claim 49.

51. The method of claim 50, wherein modulating protein synthesis comprises modulating synthesis of a target protein.

52. The method of claim 51, wherein modulating protein synthesis comprises decreasing protein synthesis.

53. The method of any one of claims 50-52, wherein the modulating protein synthesis is selective for modulating synthesis of a first protein compared to synthesis of a second protein.

54. A method of decreasing protein synthesis in a subject in need thereof or in a cell, tissue, or biological sample, comprising administering to the subject in need thereof or contacting the cell, tissue, or biological sample with an effective amount of:
a compound of any one of claims 1-48, or a pharmaceutically acceptable salt thereof;
or the composition of claim 49.

55. The method of claim 54, wherein decreasing protein synthesis comprises decreasing synthesis of a target protein

56. The method of any one of claims 54 or 55, wherein the decreasing protein synthesis is selective for decreasing synthesis of a first protein compared to synthesis of a second protein.

57. The method of any one of claims 51-53, 55, or 56, further comprising decreasing an amount of mRNA, wherein the mRNA is associated with synthesis of the target protein.

58. The method of any one of claims 51-53 or 55-57, wherein the target protein is B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D).

59. The method of any one of claims 51-53 or 55-58, wherein the target protein is MYC proto-oncogene bHLH transcription factor (MYC), anaplastic lymphoma kinase (ALK), or GTPase KRas G12D mutant (KRAS-G12D).

60. A method of treating or preventing a disease in a subject in need thereof, comprising administering to the subject in need thereof an effective amount of:
the compound of any one of claims 1-48, or a pharmaceutically acceptable salt or prodrug thereof;
or the composition of claim 49.

61. The compound of any one of claims 1-48, or a pharmaceutically acceptable salt or prodrug thereof, or the composition of claim 49, for use in treating or preventing a disease in a subject in need thereof.

62. The compound of any one of claims 1-48, or a pharmaceutically acceptable salt or prodrug thereof, or the composition of claim 49, for use in the manufacture of a medicament for treatment or prevention of a disease in a subject in need thereof.

63. The method or compound for use of any one of claims 60-62, wherein the disease is associated with B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D).

64. The method or compound for use of any one of claims 60-63, wherein the disease is associated with MYC proto-oncogene bHLH transcription factor (MYC), anaplastic lymphoma kinase (ALK), or GTPase KRas G12D mutant (KRAS-G12D).

65. The method or compound for use of any one of claims 60-64, wherein the disease is a proliferative disease.

66. The method or compound for use of claim 65, wherein the proliferative disease is cancer.

67. The method or compound for use of claim 66, wherein the cancer is prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, or neuroblastoma.

68. The method or compound for use of any one of claims 60-64, wherein the disease is a neurological disease.

69. The method or compound for use of claim 68, wherein the neurological disease is cerebellar ataxia or a neurodegenerative disease.

70. The method or compound for use of any one of claims 60-64, wherein the disease is an immune disorder.

71. The method or compound for use of claim 70, wherein the immune disorder is psoriasis, lupus, or rheumatoid arthritis.

72. A kit comprising:
the compound of any one of claims 1-48, or a pharmaceutically acceptable salt or prodrug thereof, or the composition of claim 49;
and instructions for its use.
